(19) United States
(12) Patent Application Publication
(10) Pub. No.: US 2025/0134987 A1
(43) Pub. Date: May 1, 2025

(54) COMPOSITIONS AND METHODS FOR VACCINATION AGAINST PATHOGENIC CORONAVIRUS SPECIES AND VARIANTS

(71) Applicant: Yale University, New Haven, CT (US)

(72) Inventors: Sidi Chen, New Haven, CT (US); Lei Peng, New Haven, CT (US); Zhenhao Fang, New Haven, CT (US); Jonathan Park, New Haven, CT (US); Matthew Dong, New Haven, CT (US); Xiaoyu Zhou, New Haven, CT (US); Paul Renauer, New Haven, CT (US)

(21) Appl. No.: 18/701,454

(22) PCT Filed: Oct. 14, 2022
(86) PCT No.: PCT/US2022/078125

§ 371 Date: Apr. 15, 2024

Related U.S. Application Data
(60) Provisional application No. 63/256,235, filed on Oct. 15, 2021.

(51) Int. Cl. A61K 39/215; A61K 39/00; C07K 14/005

(52) U.S. Cl. CPC A61K 39/215; C07K 14/005; A61K 2039/54; A61K 2039/543; A61K 2039/544; A61K 2039/545; A61K 2039/575

(57) ABSTRACT

The current disclosure includes coronavirus vaccines that protect against pathogenic coronavirus species, as well as their variants. In certain embodiments, SARS-CoV-2 variant specific and multivalent coronavirus vaccines are described. The vaccines typically include a modified mRNA which encodes at least one coronavirus derived immunogen, such as a spike protein or a fragment thereof. The mRNA can be encapsulated into lipid nanoparticles or other carriers and formulated as pharmaceutical compositions which can be used to generate an immune response to coronavirus in a subject. The vaccines can be used to elicit potent B and T cell responses against SARS-CoV-2 variants and to confer protective immunity against SARS-CoV-2, as well as other pathogenic coronavirus species such as SARS-CoV and/or MERS-CoV.

### CROSS-REFERENCE TO RELATED APPLICATION

The present application claims priority under 35 U.S.C. § 119 (e) to U.S. Provisional Patent Application No. 63/256,235, filed Oct. 15, 2021, which is hereby incorporated by reference in its entirety herein.

### STATEMENT REGARDING FEDERALLY SPONSORED RESEARCH

This invention was made with Government support under W81XWH-21-1-0019 awarded by the Department of Defense. The Government has certain rights in the invention.

### BACKGROUND

Severe acute respiratory syndrome coronavirus (SARS-CoV-2), the pathogen responsible for coronavirus disease 2019 (COVID-19), has caused the ongoing global pandemic. Although lipid nanoparticle (LNP)-mRNA based vaccines such as BNT162b2 (Pfizer-BioNTech) and mRNA-1273 (Moderna) have demonstrated high efficacy against COVID-19, breakthrough infections have been widely reported in fully vaccinated individuals. Moreover, the virus has continued to mutate, and multiple dangerous variant lineages have evolved, such as B.1.1.7, B.1.351, and, more recently B.1.617. The B.1.1.7 lineage (Alpha variant, or “UK variant”) has an increased rate of transmission and higher mortality. The B.1.351 lineage (Beta variant, or “South Africa variant”) has an increased rate of transmission, resistance to antibody therapeutics, and reduced vaccine efficacy. The B.1.617 lineage (“Indian variant”, including B.1.617.1 “Kappa variant”, B.1.617.2 “Delta variant” and B.1.617.3) recently emerged, and has spread rapidly and become the dominant variant in multiple regions in the world. The on-going surge of infections in the US is predominantly caused by the Delta variant, originating from the B.1.617 lineage that has greater than one-thousand-fold higher viral load in infected individuals. The B.1.617 lineage has an increased rate of transmission, shows reduced serum antibody reactivity in vaccinated individuals, and exhibits resistance to antibody therapeutics. All these variants often spread faster than the original “wildtype” (WT) virus (also noted as Wuhan-Hu-1 or WA-1), cause more severe disease, are more likely to escape certain host immune response, cause disproportionally higher numbers of breakthrough infections despite the status of full vaccination, and have been designated by WHO and CDC as “variants of concern” (VoCs). Regarding effects on vaccine efficacy, B.1.351, for example, has been known to reduce the efficacy of the Pfizer-BioNTech vaccine from >90% to near 70%. The Delta variant has also resulted in significant reduction in vaccine efficacy, especially for individuals who received only a single dose, and has caused wide-spread breakthrough infections despite the status of full vaccination.

It has been widely hypothesized that the next-generation of COVID-19 vaccines can be designed to directly target these variants (“variant-specific vaccines”). However, to date, there is no known approved or clinical stage variant-specific vaccine. Even with the two leading RNA vaccines, many questions remain regarding the efficacy, durability, modes of action, effective populations, and breadth of protection against other coronavirus strains or species. Thus, there is an ongoing need for broadly efficacious vaccines that can elicit protective immune responses against multiple coronaviruses and for a highly efficient vaccine generation platform for rapid generation of new vaccine candidates.

### BRIEF SUMMARY

The disclosure generally relates to coronavirus immunogens, and specifically, compositions and methods for vaccination against coronavirus-related illnesses.

The disclosure provides in one aspect compositions and methods for preparing and expressing immunogenic viral peptides or proteins.

The disclosure provides in one aspect provide compositions and methods for delivering nucleic acids encoding immunogenic viral peptides or proteins.

The disclosure provides in one aspect compositions and methods for vaccination against individual coronaviruses, including SARS-CoV-2 variants of concern or interest.

The disclosure provides in one aspect multivalent coronavirus vaccines that can elicit protective immunity against multiple coronavirus species and variants.

Potent coronavirus vaccines to protect against pathogenic coronavirus species, as well as their variants have been developed. The working examples demonstrate development of lipid nanoparticle-based SARS-CoV-2 variant-specific vaccines, and evaluation of the immune responses, specificity, cross-reactivity, and host cell gene expression landscapes upon vaccination. LNP-mRNA vaccine candidates that encode the B.1.351 and B.1.617 spike proteins, along with the WT spike protein were generated. With these variant-specific LNP-mRNAs, the immune responses they induce in animals against homologous (cognate) and heterologous spike antigens and SARS-CoV-2 pseudoviruses were characterized. To understand the systematic immune responses induced by variant-specific SARS-CoV-2 spike mRNA-LNP vaccination, the combined single-cell transcriptomes and lymphocyte antigen receptor repertoires of mice immunized with the vaccines were analyzed. Thus, described herein are compositions, methods, kits and devices for the design, preparation, manufacture and/or formulation of polynucleotides which encode coronavirus peptides or proteins of interest, and are useful for the generation of vaccines.

Disclosed is an isolated messenger ribonucleic acid (mRNA) containing a 5′ untranslated region (UTR), a 3′ UTR, and an open reading frame encoding a spike protein sequence that includes all or a portion of a coronavirus spike protein. The coronavirus can be SARS-CoV-2, including variants thereof. In certain embodiments, the encoded spike protein sequence includes one or more mutations that stabilize the spike protein in a prefusion conformation. For example, the spike protein sequence can include all or a portion of the S2 subunit of the spike protein, and the one or more mutations can include one or more proline substitutions in the spike S2 subunit. Exemplary proline substitutions include F817P, A892P, A899P, A942P, K986P, V987P, and combinations thereof. In certain non-limiting embodiments, the spike protein sequence contains two (dual-Pro), four (quad-Pro) or six (hexa-Pro) of the foregoing proline substitutions. In certain embodiments, the amino acid positions of the one or more mutations are indicated relative to the native or wildtype SARS-CoV-2 spike protein sequence (Wuhan-Hu-1/WA-1) set forth in SEQ ID NO:2.

In certain non-limiting embodiments, the spike protein sequence further includes a cleavage site (e.g., S1/S2 protease cleavage site and/or S2′ protease cleavage site) of the spike protein. The cleavage site can include one or more mutations to inhibit protease cleavage of the spike protein. For example, in certain non-limiting embodiments the encoded spike protein sequence further includes one or more mutations at an S1/S2 protease cleavage site, an S2′ protease cleavage site, or a combination thereof that inhibit protease cleavage of the spike protein. In certain non-limiting embodiments, the protease cleavage site is a furin cleavage site. An exemplary furin cleavage site is RRAR (SEQ ID NO:15). In certain non-limiting embodiments, the furin cleavage site is deleted or replaced with a different sequence, such as GSAS (SEQ ID NO:11), GSSS (SEQ ID NO:16), or GSGS (SEQ ID NO:17).

In certain non-limiting embodiments, the variant of SARS-CoV-2 is selected from SARS-CoV-2 B.1.1.7 (Alpha variant), SARS-CoV-2 B.1.351 (Beta variant), SARS-CoV-2 B.1.617, In certain non-limiting embodiments, the variant of SARS-CoV-2 is selected from SARS-CoV-2 B.1.617.1 (Kappa variant), SARS-CoV-2 B.1.617.2 (Delta variant) and SARS-CoV-2 B.1.617.3. In certain non-limiting embodiments, the variant of SARS-CoV-2 is selected from SARS-CoV-2 B.1.1.529/BA.1 (Omicron variant), SARS-CoV-2 BA.1, SARS-CoV-2 BA.2, SARS-CoV-2 BA.2.12.1, SARS-CoV-2 BA.4, and SARS-CoV-2 BA.5.

In certain embodiments, the open reading frame of the mRNA encodes a spike protein sequence containing the amino acid sequence of any one of SEQ ID NOs: 2-10, 34, 46-54, and 57-60, or an amino acid sequence having 75% or more sequence identity to any of one of SEQ ID NOs: 2-10, 34, 46-54, and 57-60.

Also disclosed are chimeric (hybrid) mRNAs which encode chimeric (hybrid) spike proteins having sequences from different viral species or variants. For example, disclosed is an isolated, chimeric mRNA containing a 5′ UTR, a 3′ UTR, and two or more open reading frames, wherein each open reading frame encodes a different spike protein sequence, wherein each spike protein sequence includes a spike protein subunit from the spike protein of a distinct coronavirus species or variant thereof. In certain non-limiting embodiments, the two or more open reading frames include a first open reading frame and a second open reading frame. In certain non-limiting embodiments, the first open reading frame encodes a spike S1 subunit of a first coronavirus selected from SARS-CoV, MERS-CoV, and SARS-CoV-2, and the second open reading frame encodes a spike S2 subunit of a second coronavirus selected from SARS-CoV, MERS-CoV, and SARS-CoV-2.

In particular forms, (i) the S1 subunit is from SARS-CoV-2 B.1.351 and the S2 subunit is from SARS-CoV-2 B.1.617, such as but not limited to B.1.617.2; (ii) the S1 subunit is from SARS-CoV and the S2 subunit is from SARS-CoV-2 B.1.617, such as but not limited to B.1.617.2; or (iii) the S1 subunit is from MERS-CoV and the S2 subunit is from SARS-CoV-2 B.1.617, such as but not limited to B.1.617.2. In any of the foregoing, the S2 subunit can include one or more mutations, such as the proline substitutions described above, that stabilize the spike protein in a prefusion conformation. In certain non-limiting embodiments, the chimeric mRNA does not include a linker or other domain intervening between the first and second open reading frames.

In certain non-limiting embodiments of the chimeric mRNA, each open reading frame further contains a sequence encoding a SPY tag, such as but not limited to wherein the SPY tag is positioned at the C-terminus of the spike protein subunit. In such forms, the chimeric mRNA can further include a sequence encoding a 2A self-cleaving peptide between adjacent open reading frames. In certain embodiments, the disclosure provides a chimeric mRNA wherein the two or more open reading frames include three open reading frames, wherein (i) a first open reading frame encodes an S1 subunit of a SARS-CoV-2 variant, such as but not limited to SARS-CoV-2 B.1.351; (ii) a second open reading frame encodes an S1 subunit of SARS-CoV; and (iii) a third open reading frame encodes an S1 subunit of MERS-CoV. In certain non-limiting embodiments, each open reading frame encodes a SPY tag fused to the C-terminus of the S1 subunit and/or the mRNA further includes a 2A self-cleaving peptide between adjacent open reading frames.

Further provided is an isolated mRNA containing a 5′ UTR, a 3′ UTR, and an open reading frame, wherein the open readying frame encodes an S2 subunit of a coronavirus spike protein and a SPY catcher, wherein the SPY catcher is fused to the N-terminus of the spike protein S2 subunit. The coronavirus can be SARS-CoV, MERS-CoV, SARS-CoV-2, any other pathogenic coronavirus, or a variant thereof.

Any of the disclosed mRNAs can include a 5′ cap or an analog thereof, a poly(A) tail, one or more modified nucleotides, or a combination thereof. Suitable 5′ caps or analogs thereof include, without limitation, cap0, cap1, cap2, ARCA, beta-S-ARCA, inosine, m7G, N1-methyl-guanosine, 2′-fluoro-guanosine, 7-deaza-guanosine, 8-oxo-guanosine, 2-amino-guanosine, LNA-guanosine, tri-methylgranosine (TMG), nicotinamide adenine dinucleotide (NAD), cap AG, cap AU, cap GG, and 2-azido-guanosine. Suitable modified nucleotides include, without limitation, pseudouridine, N1-methyl-pseudouridine, N1-Methylpseudouridine-5′-Triphosphate-(N-1081), 1-ethylpseudouridine, 2-thiouridine, 4′-thiouridine, 5-methoxyuridine, 5-methoxyuridine, N6-methyladenosine, and 5-methylcytosine.

In certain non-limiting embodiments, the mRNAs are codon optimized for expression in a eukaryotic cell. In certain non-limiting embodiments, the mRNAs are produced by in vitro transcription.

Also provided are isolated polynucleotides (e.g., DNA) encoding the disclosed mRNAs. The polynucleotide can include one or more promoters and/or a polyadenylation signal operably linked to a sequence encoding the mRNA. In certain non-limiting embodiments, the polynucleotide is or is contained in a plasmid. In certain non-limiting embodiments, the polynucleotide is or is contained within a vector (e.g., an expression vector). In certain non-limiting embodiments, the vector is a viral vector, such as but not limited to an adeno-associated virus (AAV) vector.

Suitable AAV serotypes include, but are not limited to, AAV1, AAV2, AAV3, AAV4, AAV5, AAV6, AAV7, AAV8, AAV9, AAV10, AAV11, AAV12, artificial variants such as AAV.rhIO, AAV.rh32/33, AAV.rh43, AAV.rh64R1, rAAV2-retro, AAV-DJ, AAV-PHP.B, AAV-PHP.S, AAV-PHP.eB, and other engineered versions of AAV. In a particular form, the AAV vector is AAV9.

Methods of using the disclosed polynucleotides are also provided. For example, described herein is a method of producing a recombinant coronavirus spike protein stabilized in a prefusion conformation. Typically, the method involves introducing an appropriate disclosed polynucleotide or vector to a host cell and incubating the host cell under conditions sufficient for expression of the polynucleotide or vector, thereby producing the recombinant spike protein. Also disclosed is a method of producing a chimeric/hybrid coronavirus spike protein by introducing an appropriate disclosed polynucleotide or vector to a host cell and incubating the host cell under conditions sufficient for expression of the polynucleotide or vector, thereby producing the chimeric/hybrid spike protein. In certain non-limiting embodiments, the foregoing methods can further include purifying the spike protein from the cell.

Also provided are virus-like particles and lipid nanoparticles containing or encapsulating a disclosed mRNA, polynucleotide (e.g., DNA molecule), vector, and/or protein encoded by a disclosed mRNA, polynucleotide (e.g., DNA molecule), or vector. In certain non-limiting embodiments, a lipid nanoparticle includes two or more distinct mRNAs, wherein each mRNA contains an open reading frame encoding all or a portion of a coronavirus spike protein derived from a distinct coronavirus species or variant thereof. For example, in certain non-limiting embodiments, a lipid nanoparticle includes three distinct mRNAs each independently encoding all or a portion of a coronavirus spike protein, wherein the coronavirus is selected from MERS-CoV, SARS-CoV, SARS-CoV-2, and variants thereof.

In certain embodiments, a lipid nanoparticle includes (i) a first chimeric mRNA encoding two or more spike protein S1 subunits each from different coronavirus species or variants, and (ii) a second mRNA encoding an S2 subunit of a coronavirus spike protein and a SPY catcher, wherein the SPY catcher is fused to the N-terminus of the spike protein S2 subunit. In certain non-limiting embodiments, each S1 subunit independently includes a SPY tag fused to the C-terminus of the S1 subunit.

In certain non-limiting embodiments, the ratio of lipid to mRNA in the disclosed lipid nanoparticles is in the range of about 5:1 to 20:1, inclusive, such as 6:1. The ratio can be a molar ratio. For example, in some certain embodiments, the N:P molar ratio of a lipid nanoparticle and mRNA is 6:1. In certain non-limiting embodiments, the lipid nanoparticle includes at least one ionizable cationic lipid, at least one helper lipid, at least one sterol, at least one PEG-modified lipid, or a combination thereof.

Suitable ionizable cationic lipids include, without limitation, 1,2-dimyristoyl-sn-glycero-3-ethylphosphocholine (DMEPC), 1,2-di-O-octadecenyl-3-trimethylammonium propane (DOTMA), 1,2-dioleoyl-3-trimethylammonium propane (DOTAP), PNI ionizable lipid, SM-102, DLin-MC3-DMA, DLin-KC2-DMA, ALC-0315, and combinations thereof in various ratios.

Exemplary helper lipids include, but are not limited to, 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3-phosphocholin (POPC) and 1,2-dioleoyl-sn-glycero-3-phosphocholine (DOPC).

Exemplary PEG-modified lipids include, but are not limited to, 1,2-dimyristoyl-racglycero-3-methoxypolyethylene glycol-2000 (PEG-DMG), 1,2-Distearoyl-sn-glycerol, methoxypolyethylene glycol (PEG-DSG), 1,2-Dipalmitoyl-sn-glycerol, methoxypolyethylene glycol (PEG-DPG), mPEG-OH, mPEG-AA (mPEG-CM), mPEG-CH₂CH₂CH₂—NH₂, MPEG-DMG, mPEG-N,N-Ditetradecylacetamide (ALC-0159), mPEG-DSPE, mPEG-DPPE, and combinations thereof in various ratios.

In certain embodiments, the sterol is cholesterol. In certain non-limiting embodiments, the sterol is a cholesterol-PEG conjugate. In certain non-limiting embodiments, the lipid nanoparticle contains about 20-60% ionizable cationic lipid, about 5-25% helper lipid, about 25-55% sterol, and/or about 0.5-15% PEG-modified lipid.

Pharmaceutical compositions and vaccines are also described. For example, disclosed is a pharmaceutical composition including a disclosed lipid nanoparticle and a pharmaceutically acceptable carrier or excipient. In certain non-limiting embodiments, a vaccine includes a disclosed lipid nanoparticle or disclosed pharmaceutical composition, optionally in combination with an adjuvant.

Methods of using the vaccines are provided. In certain non-limiting embodiments, disclosed is a method of inducing in a subject an immune response to a coronavirus, including administering to the subject a disclosed vaccine in an amount effective to generate the immune response. In certain non-limiting embodiments, the immune response is specific to MERS-CoV, SARS-CoV, or SARS-CoV-2. The immune response can include a T cell response and/or a B cell response. In certain non-limiting embodiments, the immune response involves a neutralizing antibody response specific to the coronavirus spike protein. In certain non-limiting embodiments, the immune response inhibits coronavirus infection in the subject. In certain non-limiting embodiments, the immune response inhibits replication of the coronavirus in the subject.

Vaccination can involve one or more doses or administrations of the vaccines. For example, in certain non-limiting embodiments, the subject is administered a single dose of the vaccine. In certain non-limiting embodiments, the subject is administered two or more doses of the vaccine. The two or more doses can be administered on different days, for example, 14-28 (e.g., 14, 21, or 28) days apart. In certain non-limiting embodiments, each administration of the vaccine provides a dose of about 1 μg, 3 μg, 10 μg, 25 μg, 30 μg, or 100 μg. In certain non-limiting embodiments, the effective amount of the vaccine is a total dose of about 1-500 μg, inclusive.

The vaccine can be administered by any suitable route, including via intradermal or intramuscular injection, or via oral, intranasal, or intratracheal administration.

In certain non-limiting embodiments, the subject being vaccinated has been exposed to, is infected with, or is at risk of infection by the coronavirus. In certain non-limiting embodiments, the subject is immunocompromised. In certain non-limiting embodiments, the subject is human.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the disclosure as claimed.

### BRIEF DESCRIPTION OF THE DRAWINGS

The accompanying drawings, which are incorporated in and constitute a part of this specification, illustrate several embodiments of the disclosed method and compositions and together with the description, explain certain embodiments of the disclosure.

FIG. 1A is a schematic showing the designs of three spike protein encoding mRNAs incorporated into the LNP-mRNA vaccine candidates. Functional elements are shown in the spike mRNA and translated protein of SARS-CoV-2 WT, B.1.351 and B.1.617 spikes, including protein domains, HexaPro and variant-specific mutations. FIG. 1B is a schematic showing the overall design of the primary experiments. Six-to 8-week-old C57BL/6Ncr mice (B.1.351-LNP-mRNA (top) and B.1.617-LNP-mRNA, n=6 mice per group; WT-LNP-mRNA, n=4 mice; PBS, n=9) received 1 or 10 μg of WT-LNP mRNA, B.1.351-LNP-mRNA or B.1.617-LNP-mRNA via the intramuscular route on day 0 (Prime) and day 21 (Boost). Blood was collected twice, two weeks post prime and boost. The binding and pseudovirus-neutralizing antibody responses induced by LNP-mRNA were evaluated by ELISA and neutralization assay. Mice were euthanized at day 40. The spleen, lymph node and blood samples were collected to analyze immune responses in by flow cytometry, bulk BCR and TCR profiling and single cell profiling. FIGS. 1C-1D are graphs showing serum antibody titers as determined by ELISA of WT-LNP mRNA vaccinated animals (n=4) to spike RBDs (FIG. 1C) and ECDs (FIG. 1D) of SARS-CoV-2 WT, B.1.351 and B.1.617. FIG. 1E is a graph showing serum neutralization titers of WT-LNP mRNA vaccinated animals (n=4) in a cross neutralization of SARS-CoV-2 WT, B.1.351 or B.1.617 pseudovirus infection of ACE2-overexpressed 293T cells. In FIGS. 1C-1E, two-way ANOVA with Tukey's multiple comparisons test was used to assess statistical significance. FIGS. 1F-1I are graphs showing T cell responses in WT-LNP mRNA vaccinated animals (n=4) as measured by intracellular cytokine staining 6 hours after addition of BFA for IFNγ (FIG. 1F), TNFα (FIG. 1G) and IL-2 (FIG. 1H) in CD8⁺ T cells, and IFNγ (FIG. 11) in CD4⁺ T cells. The unpaired parametric t test was used to evaluate statistical significance.

FIGS. 2A-2F show B.1.351-LNP-mRNA and B.1.617-LNP-mRNA vaccines elicit robust binding and pseudovirus-neutralizing antibody response against all three variants in mice. FIGS. 2A-2B are graphs showing serum antibody binding as determined by ELISA of B.1.351-LNP mRNA vaccinated mice against RBDs (FIG. 2A) and ECDs (FIG. 2B) of SARS-CoV-2 WT, B.1.351 and B.1.617 spikes (n=6). FIG. 2C is a graph showing serum neutralization titers of B.1.351-LNP mRNA vaccinated mice in cross neutralization of SARS-CoV-2 WT, B.1.351 or B.1.617 pseudovirus. FIGS. 2D-2E are graphs showing serum antibody binding as determined by ELISA of B.1.617-LNP mRNA vaccinated mice against RBDs (FIG. 2D) and ECDs (FIG. 2E) of SARS-CoV-2 WT, B.1.351 and B.1.617 spikes.

FIG. 2F is a graph showing serum neutralization titers of B.1.617-LNP mRNA vaccinated mice in cross neutralization of SARS-CoV-2 WT, B.1.351 or B.1.617 pseudovirus. Data are shown as mean±s.e.m. plus individual data points in dot plots. Statistical significance labels: n.s., not significant; * p<0.05; ** p<0.01; *** p<0.001; **** p<0.0001. FIG. 2G is a graph showing correlation between neutralization activity and ECD binding across various vaccination groups.

FIGS. 3A-3C are graphs showing percentage of CD8⁺ T cells from B.1.351-LNP-mRNA and B.1.617-LNP-mRNA vaccinated mice expressing IFN-γ (FIG. 3A), TNFα (FIG. 3B), and IL-2 (FIG. 3C) in response to stimulation of S peptide pools (n=3). FIGS. 3D-3E are graphs showing percentage of CD4⁺ T cells from B.1.351-LNP-mRNA and B.1.617-LNP-mRNA vaccinated mice expressing IFN-γ (FIG. 3D) and TNFα (FIG. 3E) in response to stimulation of S peptide pools (n=3). Data are shown as mean±s.e.m. plus individual data points in dot plots. Statistical significance labels: n.s., not significant; * p<0.05; ** p<0.01; *** p<0.001; **** p<0.0001.

FIG. 4A is a graph of boxplots of cell proportions by clusters for each condition (PBS, n=4; B.1.351-LNP-mRNA, n=6; B.1.617-LNP-mRNA, n=6). FIG. 4B is a graph of boxplots of cell proportions by cell type for each condition (PBS, n=4; B.1.351-LNP-mRNA, n=6; B.1.617-LNP-mRNA, n=6). The high dose (n=3 each) and low dose (n=3 each) groups for each vaccine were merged (n=6 total) in single cell data analysis. Comparison between groups was performed with Wilcoxon ranked sum test.

FIGS. 5A-5F are bar charts depicting significance values for enriched Gene Ontology biological process terms associated with upregulated genes for B.1.351-LNP-mRNA (FIG. 5A) or B.1.617-LNP-mRNA (FIG. 5B) vs PBS group in B cells, B.1.351-LNP-mRNA (FIG. 5C) or B.1.617-LNP-mRNA (FIG. 5D) vs PBS group in CD4 T cells, and B.1.351-LNP-mRNA (FIG. 5E) or B.1.617-LNP-mRNA (FIG. 5F) vs PBS group in CD8 T cells.

FIGS. 6A-6B are graph of boxplots showing Chao1 indices for each condition (PBS, n=4; B.1.351-LNP-mRNA, n=6; B.1.617-LNP-mRNA, n=6) for repertoires in the single cell BCR-seq (FIG. 6A) and single cell TCR-seq (FIG. 6B) datasets. FIGS. 6C-6F are graph of boxplots showing Chao1 (FIG. 6C) and Gini-Simpson (FIG. 6D) indices for TRA chain repertoires, and Chao1 (FIG. 6E) and Gini-Simpson (FIG. 6F) indices for TRB chain repertoires in the bulk TCR-seq dataset across vaccination and tissue of origin groups. The low dose and high dose groups of the same vaccine were grouped together.

FIG. 7A is a schematic illustration of a multivalent coronavirus (CoV) vaccine and experiments to evaluate its efficacy. A mRNA mixture of SARS-CoV-2 delta variant, SARS-CoV, and MERS was encapsulated by lipid nanoparticles and injected to mice (n=9) at day 0 (prime) and day 35 (boost). Blood samples were collected 2 weeks after prime, and 2 weeks, 2 months and 4 months after boost. Mice sera spike binding and neutralizing titers were monitored every 2 months post boost. The SARS-CoV-2 WT mRNA and delta mRNA vaccine were included as two separate control groups. Depending on the titer level after boost, a third vaccine dose can be administered to mice. The T cell response can be evaluated by flowcytometry and BCR and TCR sequencing can be performed on white blood cells from vaccinated mice. FIGS. 7B-7G are graphs showing antibody binding response curves post boost for mice vaccinated with the indicated vaccines. LNP-mRNA vaccines were prepared as described for FIG. 7A. The vaccines used multiplexed mRNA compositions including MERS-CoV either alone or in combination with SARS-CoV or SARS-CoV-2 (Delta) mixed in a 1:1 ratio and packaged into LNP. 3 ug was used for immunization/vaccination, respectively. Animals received prime and boost vaccination 21 days apart. ELISA was performed against SARS-CoV-2, SARS-CoV and MERS-CoV antigens such as RBD or ECD of their spike proteins. Results showed that the defined compositions induced robust antibody responses against the desired targeted viral species. FIGS. 7H-7M are graphs showing antibody binding response curves post boost for mice vaccinated with the indicated vaccines. LNP-mRNA vaccines were prepared as described elsewhere herein, but using the indicated multiplexed mRNA compositions (pan-1 ug/pan-3 ug: pancoronavirus 1 ug/3 ug), where three mRNAs targeting SARS-CoV-2, SARS-CoV and MERS-CoV were mixed in 1:1:1 ratio and packaged into LNP. 1 ug or 3 ug was used for immunization/vaccination. Animals received prime and boost vaccination 21 days apart. ELISA was performed against SARS-CoV-2, SARS-CoV and MERS-CoV antigens such as RBD or ECD of their spike proteins, respectively. Results showed that the pan-1 ug and pan-3 ug vaccines both induced robust antibody responses against all three viral species. FIG. 7N is a graph showing binding antibody titers measured by area under curve of OD450 response to six different spike antigens. The summary titer dot-box plots show the antibody ELISA titers of individual mice in the indicated vaccination groups.

FIGS. 8A-8B are schematic illustrations of the plasmids used in the SARS-CoV-2 variants vaccine and multivalent CoV mRNA vaccine. The figures illustrates exemplary spike protein encoding mRNAs from three CoV species and four SARS-CoV-2 strains/variants. For each CoV spike, two types of vectors for mRNA transcription (left) and pseudovirus assay (right) can be generated. In certain non-limiting embodiments, the mRNA transcription vector contains 5′ UTR, HexaPro mutations, deleted or substituted Furin cleave site, and 3′ UTR. In the pseudovirus vector, the c-terminal 19 residues can be deleted to increase pseudovirus formation.

FIG. 9 is a schematic illustration of an exemplary chimeric/hybrid CoV spike mRNA vaccine and experiments to evaluate its efficacy. In certain non-limiting embodiments, the mRNA is designed such that the S1 domain (NTD+RBD) of SARS-CoV, MERS-CoV, SARS-CoV-2 B.1.351 and B.1.617.2 are linked by the SPY tag at their C terminus in one mRNA transcript. In a separate mRNA, a SPY catcher sequence is placed at the N-terminus of the stem S2 of SARS-CoV-2 B.1.617.2 (delta variant). The LNP vaccine includes both mRNAs. After immunization in mice and upon translation, the peptides with SPY tag and SPY catcher are covalently linked and the intact S1-S2 chimeric spike antigen is formed. The immunization schedule and downstream assays for evaluating efficacy are similar to that for the multivalent coronavirus vaccine, including ELISA and neutralization assays, and flow cytometry and sequencing assays.

FIG. 10 is a schematic illustration of exemplary vectors used in the chimeric/hybrid CoV spike mRNA vaccine. Two types of vectors can be formed, either with or without the SPY conjugation system. The S2 subunit of a strong immunogenic spike can be used as backbone. In certain non-limiting embodiments, the SARS-CoV-2 delta variant S2 is used as the backbone, as illustrated. Without the SPY system, separate chimeric spike constructs are generated and evaluated in animal models (left). In certain non-limiting embodiments, when using the SPY system (right), three S1 subunits can be put in tandem and transcribed from one mRNA transcript. Upon the cleavage of the 2A linker between S1 subunit, each S1 subunit with the SPY tag is conjugated to the S2 subunit fused to the SPY catcher.

FIG. 11A is a schematic overview of an artificial intelligence (AI) based vaccine design pipeline fed on big data and deep learning. FIG. 11B is a schematic showing exemplary construct designs of several pan-coronavirus vaccine candidates. FIG. 11C is a bar graph showing pilot AAV-CoVacs production assayed by qPCR. FIG. 11D is a bar graph showing functional transduction tests assayed by FACS.

FIGS. 12A-12D are graphs showing predicted MHC-I scores for the indicated antigens. FIGS. 12E-12G are graphs showing predicted MHC-II scores for the indicated antigens. FIG. 12H is a graph showing percentage hACE2 staining as assayed via the cellular hACE2 system established for antigen testing. FIG. 12I is a graph showing FACS-based quantification of Spike-specific B cell populations performed on the splenocyte samples of the AAV vaccine injected animals in vivo. FIG. 12J is a graph showing FACS-based quantification of vaccine-induced antigen-specific IFNg+CD8 T cell populations in vivo.

FIGS. 12K-12L are graphs showing FACS-based quantification of IL7RA+ (FIG. 12K) and CD44+IL7RA+CD62L+ (FIG. 12L) T cells in vivo.

FIGS. 13A-13D illustrate the potent antibody response to Omicron BA.2, BA.2.12.1 and BA.5 subvariants by Omicron BA.2 and Delta bivalent LNP-mRNA. FIG. 13A, Vaccine design of Omicron BA.2 and Delta variant specific LNP-mRNA based on BA.2 and Delta spike mutations. Unique spike mutations on BA.2.12.1 and BA.5 (not included in LNP-mRNA) are colored in orange and magenta. FIG. 13B, Distribution of BA.2 (Yellow), BA.2.12.1 (Cyan) and BA.5 (Red) mutations in one protomer of Omicron spike trimer (PDB: 7T9K). FIG. 13C, Delta and BA.2 specific monovalent or bivalent LNP-mRNA boosters improved antibody response of WT-vaccinated mice to Omicron BA.2, BA.2.12.1 and BA.4/5 subvariants. Comparison of binding antibody titers against BA.2, BA.2.12.1 and BA.4/5 spike RBD and ECD before (D28) and after (D42) receiving 1.5 μg WT, Delta, BA.2 specific monovalent or bivalent (1.5 μg Delta+1.5 μg BA.2) LNP-mRNA boosters. Antibody titers were quantified by area under curves (AUC) of ELISA response curves in FIGS. 14A-14C and 15A-15B. Blood samples were collected in mice immunized with two doses of 1.5 μg WT LNP-mRNA followed by 1.5 μg WT, Delta, BA.2 specific monovalent or Delta & BA.2 bivalent boosters (n=6 in each group). FIG. 13D, Neutralization of Omicron BA.2, BA.2.12.1 and BA.5 pseudovirus by plasma of mice before (D28) and after (D42) vaccinated with WT, Delta, BA.2 specific monovalent or Delta & BA.2 bivalent boosters. Six samples collected on day 0 were included and compared to both D28 and D42 datasets. Titer ratios before and after receiving boosters (D42/D28 ratios) were shown in FIGS. 13C-13D. Individual dot in dot-bar plots represent value from each mouse and are shown as mean±s.e.m . . . . To assess statistical significance, two-way ANOVA with Tukey's or Šídák's multiple comparisons test was used. Statistical significance labels: * p<0.05; ** p<0.01; *** p<0.001; **** p<0.0001. Non-significant comparisons are not shown.

FIGS. 14A-14C illustrate plasma dilution-dependent ELISA response curves against WT, Delta, BA.2, BA.2.12.1 and BA4/5 spike ECDs. Plasma samples were collected at day 42 (FIG. 14A), day 28 (FIG. 14B) and day 0 (FIG. 14C) from mice immunized with WT Delta, BA.2 specific monovalent or bivalent LNP-mRNA boosters.

FIGS. 15A-15B illustrate plasma dilution-dependent ELISA response curves against WT, Delta, BA.2, BA.2.12.1 and BA4/5 spike RBDs. Plasma samples were collected at day 42 (FIG. 15A) and day 28 (FIG. 15B) from mice immunized with WT Delta, BA.2 specific monovalent or bivalent LNP-mRNA boosters.

FIG. 16 is a series of graphs showing a comparison of binding antibody titers against WT (left), Delta (Mid) and BA.2 (Right) spike RBD and ECD before (DO and D28) and after (D42) receiving 1.5 μg WT, Delta, BA.2 specific monovalent or bivalent (1.5 μg Delta+1.5 μg BA.2) LNP-mRNA boosters (n=6). Antibody titers were quantified by area under curves (AUC) of ELISA response curves in FIGS. 14A-14C and 15A-15B. The comparison with day 0 samples and insignificant comparison were not shown.

FIGS. 17A-17E is a series of graphs illustrating the comparison of ELISA antibody titers of plasma samples collected on day 0, day 28 and day 42. FIGS. 17A-17B, ELISA antibody titers against WT, Delta, BA.2, BA.2.12.1 and BA.4/5 spike RBDs before (D28, FIG. 17B) and after (D42, FIG. 17A) receiving 1.5 μg WT, Delta, BA.2 specific monovalent or bivalent (1.5 μg Delta+1.5 μg BA.2) LNP-mRNA boosters. FIGS. 17C-17E, ELISA antibody titers against WT, Delta, BA.2, BA.2.12.1 and BA.4/5 spike ECDs by plasma samples collected on (D42, FIG. 17C; D28, FIG. 17D; D0, FIG. 17E). Antibody titers were quantified by area under curves (AUC) of ELISA response curves in FIGS. 14A-14C and 15A-15B.

FIGS. 18A-18B are a series of graph illustrating a correlation of antibody titers against RBD and ECD of five spike antigens in ELISA. Antibody titers against ECD of Omicron BA.2, BA.2.12.1, BA.4/5 subvariants (left) or WT, Delta (right) were shown on y axis as log 10 AUC and plotted against corresponding RBD binding antibody titers on x axis (log 10 AUC). Titers were either shown as mean of matched vaccination group (FIG. 18A) or derived from individual animal (FIG. 18B).

FIGS. 19A-19C illustrate neutralization titration curves of serially diluted plasma collected at indicated time points from mice vaccinated with WT, Delta, BA.2 monovalent or bivalent LNP-mRNA boosters. FIG. 19A, Neutralization curves of BA.5, BA.2.12.1 and BA.2 pseudovirus by samples collected on day 42 from mice immunized with 1.5 μg WT, Delta, BA.2 monovalent or bivalent LNP-mRNA boosters. FIG. 19B, Neutralization curves of BA.5, BA.2.12.1 and BA.2 pseudovirus by samples collected on day 28 from mice immunized with two doses of 1.5 μg WT LNP-mRNA. FIG. 19C, Neutralization curves of BA.5, BA.2.12.1 and BA.2 pseudovirus by samples collected on day 0 from vaccination naïve mice. The log 10 relative light unit (RLU) measured by NanoLuc luciferase assay were shown as mean±s.e.m. and plotted against serial log 10-transformed sample dilution points.

FIGS. 20A-20C illustrate the statistical comparison of neutralizing titers of plasma samples from different vaccination groups at same time point (FIG. 20A) or against different Omicron subvariant pseudoviruses at matched time points (FIG. 20B). FIG. 20A, Omicron BA.2 (right), BA.2.12.1 (mid) and BA.5 (left) pseudovirus neutralization by plasma of mice before (D28) and after (D42) vaccinated with WT, Delta, BA.2 specific monovalent or Delta & BA.2 bivalent boosters. Six samples collected on day 0 were included and compared to both D28 and D42 datasets. FIG. 20B, BA.4/5, BA.2.12.1 and BA.2 neutralizing antibody titers from samples collected on day 0 and day 28 (WT×2) were compared. FIG. 20C, BA.4/5, BA.2.12.1 and BA.2 neutralizing antibody titers were compared within same vaccination groups at matched time points including day 28 (pre booster) and day 42 (post booster).

FIGS. 21A-21B are a series of graphs illustrating the correlation of antibody titers measured by pseudovirus neutralization and ELISA. Antibody titers determined by pseudovirus neutralization assay were shown on x axis as log 10 IC50 and plotted against ELISA binding antibody titers (log 10 AUC) measured by RBD (left) or ECD (right) spike antigens on y axis. Titer values were either derived from mean of matched vaccination group (FIG. 20B) or individual animals (FIG. 20A).

FIGS. 22A-22F illustrate design and biophysical characterization of Omicron-specific LNP-mRNA vaccine. FIG. 22A, Illustration of mRNA vaccine construct expressing SARS-CoV-2 WT and Omicron spike genes. The spike open reading frame were flanked by 5′ untranslated region (UTR), 3′ UTR and polyA tail. The Omicron mutations (red) and HexaPro mutations (black) were numbered based on WA-1 spike residue number. FIG. 22B, Distribution of Omicron spike mutations (magenta) were displayed in one protomer of spike trimer of which N-terminal domain (NTD), receptor binding domain (RBD), hinge region and S2 were colored in purple, blue, green and orange respectively (PDB: 7SBL). The HexaPro mutations in S2 were colored in cyan. FIG. 22C, Schematics illustrating the formulation and biophysical characterization of lipid nanoparticle (LNP)-mRNA. FIG. 22D, Dynamic light scattering derived histogram depicting the particle radius distribution of Omicron spike LNP-mRNA. FIG. 22E, Omicron LNP-mRNA image collected on transmission electron microscope. FIG. 22F, human ACE2 receptor binding of LNP-mRNA encoding Omicron spike expressed in 293T cells as detected by human ACE2-Fc fusion protein and PE-anti-human Fc antibody on Flow cytometry.

FIGS. 23A-23E illustrate that an omicron-specific LNP-mRNA vaccine elicited neutralizing antibodies against SARS-CoV-2 Omicron variant. FIG. 23A, Immunization and sample collection schedule. Retro-orbital blood were collected prior Omicron LNP-mRNA vaccination on day 0, day 13 and day 21. Ten mice (n=10) were intramuscularly injected with 10 μg Omicron LNP-mRNA on day 0 (prime, Omicron×1) and day 14 (boost, Omicron×2). The plasma and peripheral blood mononuclear cells (PBMCs) were separated from blood for downstream assays. The slight offset of the labels reflects the fact that each of the blood collections were perform prior to the vaccination injections. Data were collected from two independent experiments and each experiment has five mice. FIG. 23B, Binding antibody titers of plasma from mice vaccinated with Omicron LNP-mRNA against Omicron spike RBD as quantified by area under curve of log₁₀-transformed titration curve (Log₁₀ AUC) in FIG. 26. Each dot in bar graphs represents value from one mouse (n=10 mice). FIG. 23C, Neutralization of Omicron pseudovirus by plasma from Omicron LNP-mRNA vaccinated mice. FIG. 23D, Omicron live virus titration curves over serial dilution points of plasma from mice before and after immunization with Omicron LNP-mRNA at defined time points. Data of each sample were collected from three replicates (n=10 mice). FIG. 23E, Neutralization of Omicron infectious virus by plasma from Omicron LNP-mRNA vaccinated mice (n=10 mice). Data on dot-bar plots are shown as mean #s.e.m. with individual data points in plots. One-way ANOVA with Dunnett's multiple comparisons test was used to assess statistical significance. Statistical significance labels: * p<0.05; ** p<0.01; *** p<0.001; **** p<0.0001.

FIGS. 24A-24D illustrate the use of a heterologous booster with Omicron LNP-mRNA as compared to homologous booster with WT LNP-mRNA in mice that previously received a two-dose WT LNP-mRNA vaccination. FIG. 24A, Schematics showing the immunization and blood sampling schedule of mice administered with 1 μg WT LNP-mRNA prime (WT×1) and boost (WT×2) as well as 10 μg WT or Omicron-specific LNP-mRNA booster shots. The data was collected and combined from two independent experiments shown in FIGS. 27A-27D and 28A-28D. FIG. 24B, Bar graph comparing binding antibody titers of mice administered with PBS or WT and Omicron LNP-mRNA against Omicron, Delta and WA-1 RBD (ELISA antigens). The antibody titers were quantified as Log₁₀ AUC based on titration curves in FIGS. 26A-26B. PBS sub-groups (n=6 each) collected from different matched time points showed no statistical differences between each other, and were combined as one group (n=18). FIG. 24C, Pseudovirus neutralizing antibody titers in the form of log₁₀-transformed reciprocal IC50 calculated from fitting the titration curve with a logistic regression model (n=12 mice before booster, n=5 in WT×3, n=7 in WT×2+Omicron). FIG. 24D, Infectious virus neutralization titer comparisons between mice before and after vaccination with WT or Omicron boosters (n=9 mice before booster, n=5 in WT×3, n=4 in WT×2+Omicron). Titer ratios were indicated in each graph and fold change described in manuscript is calculated from (ratio-1). Data on dot-bar plots are shown as mean±s.e.m. with individual data points in plots. Two-way ANOVA with Tukey's multiple comparisons test was used to assess statistical significance. Statistical significance labels: * p<0.05; ** p<0.01; *** p<0.001; **** p<0.0001. Non-significant comparisons are not shown, unless otherwise noted as n.s., not significant. Sample number is designated as n from biologically independent samples.

FIGS. 25A-25F illustrate cross reactivity and targeting sites characterization of plasma antibodies elicited by Omicron and WT LNP-mRNAs against SARS-CoV-2 VoCs and Betacoronavirus species. FIG. 25A, cross reactivity of plasma antibody from mice immunized with Omicron LNP mRNA (prime and boost) to SARS-CoV-2 VoCs and pathogenic coronavirus species (n=10 mice). FIG. 25B, cross reactivity of plasma antibody from mice immunized with WT (WT×3) or Omicron (WT×2+Omicron) boosters to SARS-CoV-2 beta variant and pathogenic coronavirus species (n=6 mice in PBS, n=5 in WT×3, n=7 in WT×2+Omicron). FIG. 25C, representative antibodies from major classes of RBD epitopes were shown by aligning spike RBDs in each of complex structures. The Omicron RBD surface was set to semi-transparent to visualize 15 RBD mutations and their relative positions to antibody epitopes. FIG. 25D, baseline titers of plasma from mice of different vaccination status (WT×3, WT×2+Omicron, Omicron×2) were shown as log₁₀ AUC determined in hACE2 and antibody competition ELISA. Each group sample number is denoted with n (n=10 in Omicron×2, n=5 in WT×3, n=7 in WT×2+Omicron) in two independent assays (hACE2 and antibody competition ELISA). FIG. 25E, significant portion of plasma antibody from mice receiving Omicron (Omicron×2, left panel) or WT+Omicron (WT×3 middle, or WT×2+Omicron, right panel) LNP-mRNA competed with hACE2 for Omicron RBD binding in ELISA (n=10 in Omicron×2, n=5 in WT×3, n=7 in WT×2+Omicron). FIG. 25F, plasma antibody from mice receiving Omicron (Omicron×2, n=10, left panel) or WT+Omicron (WT×3, n=5, middle or WT×2+Omicron, n=7, right panel) LNP-mRNA showed various extent of binding reduction in the presence of blocking antibodies with known epitopes on RBD. The error bar and statistical information are identical with FIG. 24 and described in method section.

FIGS. 26A-25B illustrate ELISA and neutralization titration curves over serial dilution of plasma collected at different timepoints from mice administered with PBS or WT and/or Omicron LNP-mRNA. FIG. 26A, ELISA titration curves over serial log 10-transformed dilution points of plasma collected from mice before and after immunization with Omicron LNP-mRNA at defined time points (n=10). Average curves, data are shown as mean±s.e.m. FIG. 26B, Omicron pseudovirus titration curves over serial log 10-transformed dilution points of plasma collected from mice before and after immunization with Omicron LNP-mRNA at defined time points (n=10). Left panel, average curves, data are shown as mean±s.e.m.; Right panel, individual curves.

FIGS. 27A-27D illustrate both WT and Omicron specific LNP-mRNA booster shots greatly improved waning immunity of mice vaccinated with SARS-CoV-2 WT LNP-mRNA against SARS-CoV-2 Delta and Omicron variants (Independent experiment 1 or batch 1).

FIG. 27A, Schematics showing the immunization and blood sampling schedule of mice administered with 1 μg WT LNP-mRNA prime (WT×1) and boost (WT×2) as well as 10 μg WT or Omicron-specific LNP-mRNA booster shots. The plasma and PBMCs were separate from blood for downstream assays. FIG. 27B, Bar graph comparing binding antibody titers of mice administered with PBS or WT and Omicron LNP-mRNA against Omicron, Delta and WT RBD (ELISA antigens). The antibody titers were quantified as Log₁₀ AUC based on titrations curves in FIG. 26A. PBS sub-groups (n=3 each) collected from different matched time points showed no statistical differences between each other, and were combined as one group (n=9). FIG. 27C, Neutralizing antibody titers in the form of log₁₀-transformed reciprocal IC50 calculated from fitting the titration curve with a logistic regression model (n=3). FIG. 27D, Correlation of neutralization titers (log₁₀ reciprocal IC50, y axis) and ELISA titers (log₁₀ AUC, x axis) from matched vaccination group (left panel) or individual mouse (right panel). PBS samples from different timepoints were shown as one group in correlation map and were not included in linear regression model. Each dot in bar graphs represents value from one group average (left panel), or one individual mouse (right panel). Titer ratios were indicated in each graph and fold change is calculated from (ratio-1). Data on dot-bar plots are shown as mean±s.e.m. with individual data points in plots. Two-way ANOVA with Tukey's multiple comparisons test was used to assess statistical significance. Statistical significance labels: * p<0.05; ** p<0.01; *** p<0.001; **** p<0.0001. Non-significant comparisons are not shown, unless otherwise noted as n.s., not significant.

FIGS. 28A-28D illustrate that omicron specific LNP-mRNA booster shots greatly improved waning immunity of mice vaccinated with SARS-CoV-2 WT LNP-mRNA against SARS-CoV-2 Delta and Omicron variants (Independent experiment 2 or batch 2). FIG. 28A, Schematics showing the immunization and blood sampling schedule of mice administered with 1 μg WT LNP-mRNA prime (WT×1) and boost (WT×2) as well as 10 μg Omicron-specific LNP-mRNA booster shots. The plasma and PBMCs were separate from blood for downstream assays. FIG. 28B, Bar graph comparing binding antibody titers of mice administered with PBS or WT and Omicron LNP-mRNA against Omicron, Delta and WT RBD (ELISA antigens). The antibody titers were quantified as Log 10 AUC based on titrations curves in FIG. 26A. PBS sub-groups (n=3 each) collected from different matched time points showed no statistical differences between each other, and were combined as one group (n=6). FIG. 28C, Neutralizing antibody titers in the form of log 10-transformed reciprocal IC50 calculated from fitting the titration curve with a logistic regression model (n=9 before booster, n=5 in WT×3, n=4 in WT×2+Omicron). FIG. 28D, Correlation of neutralization titers (log 10 reciprocal IC50, y axis) and ELISA titers (log 10 AUC, x axis) from matched vaccination group (left panel) or individual mouse (right panel). PBS samples from different timepoints were shown as one group in correlation map and were not included in linear regression model. Each dot in bar graphs represents value from one group average (left panel), or one individual mouse (right panel). Titer ratios were indicated in each graph and fold change is calculated from (ratio-1). Data on dot-bar plots are shown as mean±s.e.m. with individual data points in plots. Two-way ANOVA with Tukey's multiple comparisons test was used to assess statistical significance. Statistical significance labels: * p<0.05; ** p<0.01; *** p<0.001; **** p<0.0001. Non-significant comparisons are not shown, unless otherwise noted as n.s., not significant.

FIGS. 29A-29B illustrate ELISA titration curves over serial dilution of plasma collected at different timepoints from mice administered with PBS or WT and/or Omicron LNP-mRNA. FIG. 29A, ELISA titration curves of batch 1 experiment (n=3). FIG. 29B. ELISA titration curves of batch 2 experiment (n=9 before booster, n=5 in WT×3, n=4 in WT×2+Omicron). The OD450 values were plotted against a series of log 10-transformed dilution points of plasma from mice 35 days post WT prime, >4 months post WT prime (day 127 in batch 1 and day 166 in batch 2) and 2 weeks post booster (day 140 in batch 1 and day 180 in batch 2) of WT or Omicron LNP-mRNA, against spike receptor binding domain (RBD) antigens of Omicron variant (left), Delta (mid) and WT (right) were shown. Data are shown as mean±s.e.m. in plots.

FIG. 30 illustrates binding antibody titers of mice administered with PBS or WT and Omicron LNP-mRNA against Omicron, Delta and WT RBD (ELISA antigens), were grouped by vaccination timepoints to compare titers against different RBD antigens. The antibody titers were quantified as area under curve of log 10-transformed titration curve (Log 10 AUC). The data were derived from independent experiment 1 and 2.

FIGS. 31A-31E illustrate Omicron, Delta, and WT pseudovirus production, characterization, and neutralization assay. FIG. 31A. Functional titration curves of Omicron, Delta, and WA-1 pseudoviruses in hACE2+ cells. FIG. 31B. Representative Flow Cytometry plots of infectivity of Omicron, Delta, and WA-1 pseudoviruses in hACE2+ cells. FIG. 31C. Quantification of infectivity of Omicron, Delta, and WT pseudoviruses in hACE2+ cells (n=4). FIG. 31D, Neutralization titration curves from batch 1 experiment (n=3). FIG. 31E. Neutralization titration curves from batch 2 experiment (n=9 before booster, n=5 in WT×3, n=4 in WT×2+Omicron). Percent of pseudovirus infected cells was plotted over serial dilutions of plasma from mice 35 days post WT prime, >4 months post WT prime (day 127 in batch 1 and day 166 in batch 2) and 2 weeks post booster (day 140 in batch 1 and day 180 in batch 2) of WT and Omicron LNP-mRNA against Omicron (left), Delta (mid) and WT (right) pseudovirus. Pseudovirus infection rate was calculated from percent of GFP positive cells and was plotted against plasma dilution (log 10 transformed) as titration curve. Top panels, average curves, data are shown as mean±s.e.m.; Bottom panels, individual curves. Sample number is designated as n from biologically independent samples. One-way ANOVA with Holm-Sidak multiple comparisons test was used to assess statistical significance. Statistical significance labels: * p<0.05; ** p<0.01; *** p<0.001; **** p<0.0001.

FIGS. 32A-32B illustrate neutralizing antibody titers in the form of log 10-transformed reciprocal IC50 were grouped by vaccination timepoints to compare titers against different pseudoviruses. The neutralization titers from combined datasets (FIG. 32A) or batch 1 (FIG. 32B) were quantified as log 10-transformed reciprocal IC50 values (Log 10 reciprocal IC50, or Log 10 IC50) based on titration curves in FIG. 31. Titer ratios were indicated in each graph and fold change is calculated from (ratio-1). Data on dot-bar plots are shown as mean±s.e.m. with individual data points in plots. Two-way ANOVA with Tukey's multiple comparisons test was used to assess statistical significance. Statistical significance labels: * p<0.05; ** p<0.01; *** p<0.001; **** p<0.0001. Non-significant comparisons are not shown, unless otherwise noted as n.s., not significant.

FIGS. 33A-33B illustrate live virus neutralization titration curves over serial dilution of plasma collected at different timepoints from mice administered with PBS or WT and/or Omicron LNP-mRNA. FIG. 33A, Omicron live virus titration curves (n=9 before booster, n=5 in WT×3, n=4 in WT×2+Omicron). FIG. 33B. Delta live virus titration curves (n=9 before booster, n=5 in WT×3, n=4 in WT×2+Omicron). Titration curves were plotted over serial dilution points of plasma collected from mice before and after WT or Omicron LNP-mRNA boosters at defined time points. Data of each sample were collected from two replicates. Data are shown as mean±s.e.m. in plots.

FIGS. 34A-34C illustrate a Correlation analysis of antibody titers determined by ELISA, pseudovirus neutralization and live virus neutralization assays. FIG. 34A, Correlation between pseudovirus neutralization titers (log 10 reciprocal IC50, y axis) and ELISA titers (log 10 AUC, x axis) from matched vaccination group (left panel) or individual mouse (right panel). FIG. 34B. Correlation between live virus neutralization titers (log 10 IC50, x axis) and ELISA titers (log 10 AUC, y axis) from matched vaccination group (left panel) or individual mouse (right panel). FIG. 34C. Correlation between live virus neutralization titers (log 10 IC50, x axis) and pseudovirus neutralization titers (log 10 AUC, y axis) from matched vaccination group (left panel) or individual mouse (right panel). PBS samples from different timepoints were shown as one group in correlation map and were not included in linear regression model. Each dot in bar graphs represents value from one group average (left panel), or one individual mouse (right panel). The Prism default two-side simple linear regression test without multiple comparison adjustment was used to assess statistical significance.

FIGS. 35A-35C illustrate an assessment of WT or Omicron LNP-mRNA mediated cross reactivity against a panel of SARS-CoV-2 variants and pathogenic coronavirus species in ELISA. FIG. 35A, binding antibody titers (Log 10 AUC) of plasma from mice that received Omicron LNP-mRNA prime and boost (Omicron×2, n=10). FIG. 35B, binding antibody titers of plasma from mice that received WT (WT×3, n=5) or Omicron (WT×2+Omicron, n=7) LNP-mRNA boosters. FIG. 35C, binding antibody titers of plasma from mice that received Omicron LNP-mRNA prime+boost (Omicron×2, n=10), WT (WT×3, n=5) or Omicron (WT×2+Omicron, n=7) LNP-mRNA boosters. This figure is a combination of data from the experiment shown in FIGS. 22A-22F and 24A-24D for comparison clarity. Data on dot-bar plots are shown as mean±s.e.m. with individual data points in plots. Two-way ANOVA with Tukey's multiple comparisons test was used to assess statistical significance. Multiple comparisons between titers against different ELISA antigens were made within same vaccination group. All comparisons with MERS RBD were significant and not shown in graph to simplify comparisons. Statistical significance labels: * p<0.05; ** p<0.01; *** p<0.001; **** p<0.0001. Non-significant comparisons are not shown.

FIGS. 36A-36C illustrate competition ELISA titration curves and binding antibody titers against low-density Omicron RBD from mice vaccinated with WT and/or Omicron LNP-mRNA. FIG. 36A. hACE competition ELISA titration curves over a series of log 10-transformed dilution points of plasma from mice vaccinated with Omicron LNP-mRNA (Omicron×2 plasma, left, n=10) or WT/Omicron LNP-mRNA (WT×3, middle, n=5 and WT×2+Omicron plasma, right, n=7). FIG. 36B. antibody competition ELISA titration curves over a series of log 10-transformed dilution points of plasma from mice vaccinated with Omicron LNP-mRNA (Omicron×2 plasma, left, n=10) or WT/Omicron LNP-mRNA (WT×3 plasma, middle, n=5 and WT×2+Omicron plasma, right, n=7). FIG. 36C. PBS buffer as negative control to show minimal cross reactivity of anti-mouse secondary antibody with human IgG blocking antibodies, including Clone 13A, CR3022 and S309. n=2 and each contains 8 mock dilution points.

FIG. 37 is a representative flow cytometry gating strategy for detecting Omicron spike binding to human ACE2 receptor.

FIGS. 38A-38I illustrate antibody responses induced by Triplex LNP-mRNA vaccination against SARS-CoV-2 Delta, SARS-CoV and MERS-CoV in vivo. FIG. 38A, Schematics of mRNA vaccine construct design against pathogenic human coronavirus species. Each construct has regulatory elements (5′UTR, 3′UTR and polyA) and spike ORF. The domain structures as well as engineered mutations of translated spike proteins of SARS-CoV-2 Delta variant (Delta), SARS-CoV (SARS) and MERS-CoV (MERS). FIG. 38B, Engineered mutations in spike protein structures of SARS-CoV-2 Delta, SARS-CoV and MERS-CoV. The N-terminal domain (NTD, blue), receptor binding domain (RBD, green) and S2 subunit (orange) of one protomer along with homologous HexaPro mutations (pink) and Delta variant mutations (red) were highlighted in the spike trimer structures. FIG. 38C, Schematics of characterization of LNP-mRNA vaccine formulations. Assembly procedure of LNP-mRNA vaccine on NanoAssemblr Ignite and downstream biophysical characterization assays. FIG. 38D, Histogram displaying radius distribution of LNP-mRNA formulations of SARS-CoV-2 Delta and a Triplex (Delta+SARS+MERS) (abbreviated as Triplex-CoV or MixCoV), measured by dynamic light scattering (DLS). The polydispersity index and mean radius of each LNP sample were shown at top left corner. FIG. 38E, Transmission electron microscope (TEM) images of Delta and Triplex-CoV LNP-mRNAs. FIG. 38F, Schematics of vaccination schedule of the Triplex LNP-mRNA formulations, as well as downstream assays to evaluate the antibody responses and other immunological profiles. FIG. 38G, Binding antibody titers of plasma samples from mice administered with PBS or different LNP-mRNAs (n=9 mice from one independent experiment) against RBD or ectodomain (ECD) of SARS-CoV-2 wild type (WT, Wuhan/WA-1), Delta variant, SARS and MERS spikes. The binding antibody titers were quantified by area under curve of log 10-transformed titration curve (log 10 AUC) in FIGS. 44A-44D. The mice were intramuscularly injected with two doses (×2, 2 weeks apart) of PBS, 1 μg SARS-CoV-2 Delta variant LNP-mRNA (delta), 1 μg or 3 μg equal mass mixture of Delta, SARS and MERS LNP-mRNA (Triplex-CoV). FIG. 38H, Overall heatmap of antibody titers of individual mice (one column represents one mouse, n=9) against eight spike antigens in ELISA (one row represents one antigen). FIG. 38I, Correlation of antibody titers against RBD (y value) and ECD (x value) of same coronavirus spike, by individual mouse, or by averaged group (n=9 mice×4 antigens). In the dot-box plots of this figure, each dot represents data from one mouse. Data are shown as mean±s.e.m. plus individual data points in plots. Two-way ANOVA with Tukey's multiple comparisons test was used to assess statistical significance. Statistical significance labels: * p<0.05; ** p<0.01; *** p<0.001; **** p<0.0001. Non-significant comparisons are not shown, unless otherwise noted as n.s., not significant.

FIGS. 39A-39E illustrate neutralizing antibody responses induced by Triplex LNP-mRNA vaccination against SARS-CoV-2 Delta, SARS-CoV and MERS-CoV in vivo. FIG. 39A, Neutralization titration curves of plasma from mice treated with PBS, Delta, Triplex-CoV LNP-mRNA against WT and Delta SARS-CoV-2, SARS-CoV and MERS-CoV pseudoviruses. The percent of GFP positive cells reflected the infection rate of host cells by pseudovirus and was plotted against the dilution factors of mice plasma to quantify neutralizing antibody titers. FIG. 39B, Neutralizing antibody titers in the form of reciprocal IC50 derived from fitting the titration curves with a logistic regression model. Each dot represents data from one mouse and each group contains nine mice (n=9, one independent experiment). FIG. 39C, Neutralization assay using authentic virus in BL3 setting. Neutralization curves and titer quantification dot plots (n=9). FIG. 39D. Correlation of neutralization log 10 IC50 vs. antibody titers against ECD of same coronavirus spike, by individual mouse, or by averaged group (n=9 mice×4 antigens). FIG. 39E, Correlation between BL3 authentic virus neutralization and BL2 pseudovirus neutralization, and between BL3 authentic virus neutralization and ELISA, by individual mouse (n-9 mice×1 antigens). In the dot-box plots of this figure, each dot represents data from one mouse. Data are shown as mean #s.e.m. plus individual data points in plots. Two-way ANOVA with Tukey's multiple comparisons test was used to assess statistical significance. Statistical significance labels: * p<0.05; ** p<0.01; *** p<0.001; **** p<0.0001. Non-significant comparisons are not shown, unless otherwise noted as n.s., not significant.

FIGS. 40A-40G illustrate in vivo antibody responses induced by Duplex LNP-mRNA vaccination against MERS-CoV, in combination with SARS-CoV-2 Delta or SARS-CoV. FIG. 40A, Schematics of vaccination schedule of the MERS Singlet and Duplex combo LNP-mRNA formulations, as well as downstream assays to evaluate the antibody responses and other immunological profiles. Two Duplexes were evaluated, (MERS+SARS) or (MERS+SARS2 Delta). FIG. 40B, Dot-box plots summarizing binding antibody titers of plasma from mice administered with PBS or different LNP-mRNAs (n=3 mice, one independent experiment) against RBD or ECD of SARS-CoV-2 WT/WA-1 and Delta variant, as well as SARS and MERS spikes. FIG. 40C, Heatmap of antibody titers of individual mice (one column represents one mouse, n=3) against eight spike antigens in ELISA (one row represents one antigen FIG. 40D, Correlation of antibody titers against RBD (y value) and ECD (x value) of same coronavirus spike, by individual mouse, or by averaged group (n=3 ×4 antigens). FIG. 40E, Neutralization titration curves of plasma from mice treated with PBS control, or LNP-mRNA formulations with MERS alone or in Duplexes (MERS+SARS) or (MERS+SARS2 Delta); all tested against WT/WA-1 and Delta SARS-CoV-2, SARS-CoV and MERS-CoV pseudoviruses. The percent of GFP positive cells reflected the infection rate of host cells by pseudovirus and was plotted against the dilution factors of mice plasma to quantify neutralizing antibody titers (n=3). FIG. 40F, Neutralizing antibody titers in the form of reciprocal IC50 derived from fitting the titration curves with a logistic regression model. Each dot represents data from one mouse and each group contains three mice (n=3). FIG. 40G, Correlation of neutralization IC50 vs. antibody titers against ECD of same coronavirus spike, by individual mouse, or by averaged group (n=3×4 antigens). In the dot-box plots of this figure, each dot represents data from one mouse. Data are shown as mean±s.e.m. plus individual data points in plots. Two-way ANOVA with Tukey's multiple comparisons test was used to assess statistical significance. Statistical significance labels: * p<0.05; ** p<0.01; *** p<0.001; **** p<0.0001. Non-significant comparisons are not shown, unless otherwise noted as n.s., not significant.

FIGS. 41A-41E illustrate single cell transcriptomics of animals vaccinated by multiplexed LNP-mRNA vaccine against SARS-CoV-2, SARS-CoV and MERS-CoV in mice. FIG. 41A UMAP visualization of all 91,526 cells pooled across samples and conditions. All identified clusters are shown with cell identities assigned, based on the expression of cell type specific markers. FIG. 41B UMAP visualization, colored by vaccination groups. PBS, Delta, mixCoV-lo (i.e. Triplex 1 μg) and mixCoV-hi (i.e. Triplex 3 μg). n=3 mice, one independent experiment. FIG. 41C Heatmap showing the population clusters with distinct expression patterns. Rows represent the scaled expression of the top 10 genes that were differentially expressed in each cluster, relative to all other cells, based on Wilcoxon rank sum analysis. FIG. 41D Stacked bar plot depicting the proportion of different immune populations for each vaccination group. FIG. 41E Dot-whisker plot of immune cell proportions by cell type for each vaccination group: PBS, Delta, mixCoV-lo and mixCoV-hi; n=3 mice each group. Statistical differences were assessed by two-way ANOVA with Tukey's correction for multiple testing. Statistical significance labels: * p<0.05; ** p<0.01; *** p<0.001; **** p<0.0001. Non-significant comparisons are not shown, unless otherwise noted as n.s., not significant.

FIGS. 42A-42E illustrate direct comparison of sequential vs. mixture vaccination schedules against SARS-CoV-2 Delta, MERS-CoV, and SARS-CoV. FIG. 42A Schematics of sequential vs. mixture vaccination schedules and sampling. In the Sequential vaccination schedule, vaccinations of SARS-CoV-2 Delta, MERS-CoV, and SARS-CoV were given in sequence separated by 3 weeks, each with 1 μg LNP-mRNA prime and 1 μg LNP-mRNA boost 3 weeks apart. In the Mixture vaccination schedule, vaccinations of SARS-CoV-2 Delta, MERS-CoV, and SARS-CoV were given simultaneously, each at 1 μg LNP-mRNA (3 μg total) for both prime and boost. The first dose and the blood sample harvest were done at the same day for both sequential and mixture schedules for comparison. FIG. 42B Dot-box plots summarizing binding antibody titers of plasma from mice administered with PBS, Sequential or Mixture LNP-mRNA vaccinations (n=4 mice, one independent experiment) against RBD or ECD of SARS-CoV-2 WT/WA-1 and Delta variant, as well as SARS and MERS spikes. FIG. 42C Neutralization titration curves of plasma from mice treated with PBS, Sequential or Mixture LNP-mRNA vaccinations (n=4 each, one independent experiment); all tested against WT/WA-1 and Delta SARS-CoV-2, SARS-CoV and MERS-CoV pseudoviruses. The percent of GFP positive cells reflected the infection rate of host cells by pseudovirus and was plotted against the dilution factors of mice plasma to quantify neutralizing antibody titers. FIG. 42D Neutralizing antibody titers in the form of reciprocal IC50 derived from fitting the titration curves with a logistic regression model. Each dot represents data from one mouse and each group contains three mice (n=4). FIG. 42E Blocking ELISA antibody titers of plasma from different vaccination groups against Delta (left), SARS (mid), MERS (right) ECDs in the presence of competing reagents including PBS (negative control), Delta, SARS or MERS ECDs. Statistical significance was analyzed between groups of different blockers (n=4, one independent experiment). In the dot-box plots of this figure, each dot represents data from one mouse. Data are shown as mean±s.e.m. plus individual data points in plots. Two-way ANOVA with Tukey's multiple comparisons test was used to assess statistical significance. Statistical significance labels: * p<0.05; ** p<0.01; *** p<0.001; **** p<0.0001. Non-significant comparisons are not shown, unless otherwise noted as n.s., not significant.

FIGS. 43A-43D illustrate sequence alignment, functional validation and ELISA titration curves for engineered mRNA-encoded spike proteins of three pathogenic human coronavirus species. FIG. 43A Sequence alignment of spikes of SARS-CoV-2 Delta variant, SARS-CoV and MERS-CoV used in the LNP-mRNA vaccine. The full-length spike sequences of these three pathogenic human coronavirus species were aligned and their degree of identity at each residue was color coded by a gradient blue color. FIGS. 43B-43C) Surface expression of functional spike proteins in 293T cells after electroporation of corresponding mRNA, as detected by human ACE2 (FIG. 43B) or human DPP4 (FIG. 43C) Fc fusion protein bound to PE anti-Fc antibody. FIGS. 43D-43E ELISA titration curves over serial log 10-transformed dilution points of plasma samples from mice treated with spike antigens of SARS2 WT/WA1, SARS2 Delta, SARS and MERS. RBD and ECD ELISA spike antigens were used to evaluate the potency of binding antibodies induced by LNP-mRNA vaccines (top and bottom four panels, respectively). The mice were intramuscularly injected with two doses (×2, 2 weeks apart) of the following: (FIG. 43D) PBS, 1 μg SARS-CoV-2 Delta variant LNP-mRNA (delta), 1 μg or 3 μg equal mass mixtures (Delta, SARS and MERS mRNA) delivered by LNP (Triplex-CoV); (FIG. 43E) PBS, 3 μg MERS LNP-mRNA, 3 μg equal mass mixture of MERS mRNA in combination with SARS or Delta mRNA delivered by LNP (Comb).

FIGS. 44A-44D illustrate single cell transcriptomics visualization, clustering and cell type identification. (FIG. 44A) UMAP visualization, colored by the scaled expression of representative cell type-specific markers in T cells, NK cells, myeloid cells, B cells, and plasma cells. (FIG. 44B) Bubble plots showing cell population clusters and their respective feature markers. (FIG. 44C) UMAP clustering, color-coded by major immune cell populations. (FIG. 44D) UMAP visualizations of sub-clustering, performed in pooled B cells, plasma cells, myeloid cells, and activated CD4 T cells. Cell subclusters were identified as the indicated immune populations using the markers presented in the main Figures.

FIGS. 45A-45B illustrate additional pathway analysis of differentially expressed genes compared between vaccination groups in different cell types in the single cell RNA-seq data. (FIG. 45A) Bubble plots of overall biological process pathways of differentially expressed genes compared between vaccination groups in different cell types. Each dot is a pathway presented with a color and size that represent the respective log fold change and −log 10 adjusted p value, while the dot position compares the activation score (mean expression log fold change of pathway genes) in the analysis of mixCoV-vs-PBS (y axis), relative to the Delta-vs-PBS (x-axis). (FIG. 45B) Network plots of enriched pathways of differentially expressed genes between the vaccination groups and PBS, in different cell types. Each dot is a pathway with the size and color representing the −log 10 adjusted p value and the pathway cluster, respectively. Clusters are labeled with the most significantly enriched member pathway (meta-pathway). Colored representative meta-pathway clusters correspond to the colored text boxes.

FIGS. 46A-46C illustrate differential expression, pathway signature and gene set cluster analyses of single cell transcriptomics for animals vaccinated by multiplexed LNP-mRNAs. (FIG. 46A) Square plots compare differential expression (DE) of mixCoV-vs-PBS (y axis) to Delta-vs-PBS (x axis) analyses (n=3, one independent experiment). Each gene is presented by a dot, positioned by the log 2 (x+1) fold change in either DE analysis and sized by the −log 10 FDR-adjusted p value. Genes that are upregulated or down regulated in mixCoV-vs-PBS are shown as red or blue dots, respectively. Analyses were done for B cell, CD4 T cell and CD8 T cell populations. (FIG. 46B) Ridge density plots showing the expression log fold change meta-pathway genes between different vaccination groups in different cell types. Each plot presents the top five meta-pathways in either mixCoV-vs-PBS analysis, and only differentially expressed genes of either analysis were selected for each meta-pathway ridgeplot. (FIG. 46C) Heatmaps of differentially expressed genes between different vaccination groups of representative pathways in different cell types.

FIGS. 47A-47D illustrate analyses of antibody responses induced by sequential and Triplex LNP-mRNA vaccinations. (FIGS. 47A-47B) ELISA OD450 titration curves over serial log 10-transformed dilution points of plasma from mice treated with PBS, sequential or mixture LNP-mRNA vaccinations. ELISA antibody titers are against RBDs or ECDs of SARS2 WT/WA1, SARS2 Delta, SARS and MERS. The Sequential vaccination mice were intramuscularly injected with two doses (×2, 3 weeks between prime and boost) of 1 μg SARS-CoV-2 Delta, MERS, SARS LNP-mRNA, three weeks apart, in this sequence (Sequential Delta-MERS-SARS). The Mixture vaccination mice were intramuscularly injected with two doses (3 weeks between prime and boost) 3 μg equal mass mixture (1 μg each) of Delta, SARS and MERS LNP-mRNA (Mixture Delta/MERS/SARS). (FIGS. 47C-47D) Comparative analyses of antibody responses induced by Triplex LNP-mRNA vaccination against SARS-CoV-2 Delta, SARS-CoV and MERS-CoV in vivo. ELISA antibody titers are against (FIG. 47C) RBDs or (FIG. 47D) ECDs of SARS2 WT/WA1, SARS2 Delta, SARS and MERS.

FIGS. 48A-48D illustrate a correlation analysis of neutralization datasets; Blocking ELISA titration curves. (FIG. 48A) Heatmap of antibody titers of individual mice (one column represents one mouse) against eight spike antigens in ELISA (one row represents one antigen). (FIG. 48B) Correlation of antibody titers against RBD (y value) and ECD (x value) of same coronavirus spike, by individual mouse, or by averaged group. (FIG. 48C) Correlation of neutralization IC50 vs. antibody titers against ECD of same coronavirus spike, by individual mouse, or by averaged group.

(FIG. 48D) Blocking ELISA titration curve in response to the Delta, SARS or MERS ECD antigen in the presence of various competing agents or blockers: PBS, Delta ECD, SARS ECD and MERS ECD.

FIGS. 49A-49B illustrate blocking ELISA antibody titers of plasma from different vaccination groups. (FIG. 49A) Blocking ELISA antibody titers against Delta, SARS, and MERS ECDs in the presence of competing reagents including PBS (negative control), Delta, SARS or MERS ECDs. Statistical significance was analyzed between different vaccination groups in the presence of the same blocker. PBS plasma group was excluded in the statistical analysis in order to simplify graph. (FIG. 49B) Normalized blocking effect induced by different blockers in each vaccination group in response to ELISA antigens of Delta, SARS and MERS ECDs. The blocking effect was quantified by normalizing the blocker-induced AUC reduction with vaccine-specific AUC increase. The vaccine-specific AUC increase (100%) is calculated from AUC difference in PBS plasma group (0% or baseline) and vaccination group under the same antigen and blocker condition. The blocker-induced AUC reduction is the AUC difference between PBS and blocker treatment under the same vaccination and antigen condition.

FIGS. 50A-50E illustrate that full-length spike and RBD-oligomer LNP mRNAs elicited significant and distinct antibody responses to Omicron BA.2 and BA.5. FIG. 50A, antigen design of two spike full lengths (WT and BA.2) and three RBD oligomers. BA.2 RBD oligomers contain N-term signal peptide and C-term trimer foldon with or without virus-like particle (VLP) sequences including ferritin or human PEG10. FIG. 50B, structures of Omicron full length spike and spike RBD trimer (fibritin trimer foldon, PDB: 1RFO) on a ferritin nanoparticle (PDB: 5C6F). FIG. 50C, Significant binding antibody titers against BA.2 and BA.5 RBDs elicited by full-length spikes and RBD oligomers. Mice (n=5 in each group) were immunized with 5 μg spike full length or RBD oligomer LNP mRNAs on day 0 (prime) and day 14 (boost). Blood samples were collected on day 0 (pre-vaccination) and day 28 (2-week post boost). ELISA antibody titers were shown as area under curve (AUC) of dose response curves in supplementary FIG. 2. FIG. 50D, full length spike and RBD oligomer LNP mRNAs elicited neutralizing antibodies against BA.2 and BA.5. FIG. 50E, BA.2 (left) and BA.5 (right) binding and neutralizing titer correlation analysis. Only significant comparisons between day 28 groups were shown. Each dot in bar groups represents one mouse sample. Data are shown as mean #s.e.m.

FIGS. 51A-51E illustrate the size distribution of LNP mRNAs as characterized by dynamic light scattering. Graphs represent WT full length mRNA (FIG. 51A), BA.2 full length LNP mRNA (FIG. 51B), BA.2-RBD-ferritin LNP mRNA (FIG. 51C), BA.2 RBD-PEG10 LNP mRNA (FIG. 51D), and BA.2-RBD-ferritin trimer-LNP mRNA (FIG. 51E).

FIGS. 52A-52B illustrate plasma dilution-dependent response curves of BA.2 or BA.5 spike RBD binding antibodies in ELISA. FIGS. 52A-52B, BA.2 (left) or BA.5 (right) RBD binding OD450 response curves of serially diluted plasma collected on day 0 (FIG. 52A) and day 28 (FIG. 52B).

FIGS. 53A-53B illustrate plasma dilution-dependent curves of infection rate as quantified by normalized log₁₀ relative luminescence unit (RLU). FIGS. 53A-53B, Plasma dilution dependent infection curves of BA.2 (FIG. 53A) and BA.5 (FIG. 53B) pseudovirus neutralized by mice plasma before (day 0) or after two doses (day 28) of full-length spike or RBD oligomer LNP mRNA.

FIGS. 54A-54B illustrate a comparison of binding (FIG. 54A) and neutralizing (FIG. 54B) antibody titers against BA.2 vs. BA.5 antigens or pseudoviruses.

### DETAILED DESCRIPTION

The disclosed method and compositions may be understood more readily by reference to the following detailed description of certain embodiments and the Examples included therein and to the Figures and their previous and following description.

The coronavirus disease 2019 (COVID-19) pathogen, severe acute respiratory syndrome coronavirus (SARS-CoV-2), has infected over hundreds of millions of individuals, resulting in millions of deaths around the globe. Protective vaccines are critical to control the on-going COVID-19 pandemic as well as potential future outbreaks of emerging pathogenic coronaviruses. Lipid nanoparticle (LNP) based mRNA COVID-19 vaccines offer strong protection against SARS-CoV-2. However, multiple dangerous variant lineages have evolved, such as B.1.1.7, B.1.351, and B.1.617. These variants spread faster, cause more severe disease, can escape some host immunity, and cause high numbers of breakthrough infections. For example, the Delta variant has resulted in significant reduction of vaccine efficacy especially for those who received only a single dose, where early reports indicated the efficacy can be as low as only 33%. The significantly reduced efficacy partly explains why the Delta variant has caused wide-spread breakthrough infections despite the status of full vaccination.

In addition, pathogenic coronaviruses may continue to emerge around the world, in part due to the close contacts between humans and many wildlife species. New pathogenic viral strains or species may potentially give rise to future outbreaks or even pandemics. There are currently two recently authorized vaccines for SARS-CoV-2, however, there is no vaccine for other pathogenic coronavirus species, such as SARS-CoV and MERS-CoV. Even with the two leading RNA vaccines, many critical questions remain regarding the efficacy, durability, modes of action, effective populations, and breadth of protection against other coronavirus strains or species. Further, there has been no report or immunology study on a variant-specific vaccine to date. There is a need for more than one tool to be ready for such natural biological threats. These can be (1) broadly efficacious vaccines that can elicit protective immune responses against multiple coronaviruses; or (2) a highly efficient vaccine generation platform that is able to rapidly turnaround new vaccine candidates. Thus, our world needs multiple powerful pipelines with the ability to rapidly design, develop, test and characterize of novel vaccine candidates, to enable swift response to new and emerging pathogenic coronavirus strains or species.

The working Examples demonstrate the development of intelligent and potent coronavirus vaccines to protect against pathogenic coronavirus species, as well as their variants. LNP-mRNA vaccines were generated with mRNAs specifically encoding the B.1.351, B.1.617, and wildtype (WT) SARS-CoV-2 spikes, and animal models were used to systematically study the induced immune response. Mice receiving the LNP-mRNA spike vaccines developed dose-dependent and prime-boost-dependent antibody responses, including serum reactivity to receptor binding domains (RBDs) and full ectodomains (ECDs) of all three spikes, as well as potent neutralization activities. However, sera from mice receiving WT-LNP-mRNA showed significant reduction of neutralization ability against both B.1.351 and B.1.617. In contrast, sera from B.1.617-LNP-mRNA vaccinated mice showed strongest neutralization ability against the cognate B.1.617 spike. Sera from B.1.351-LNP-mRNA vaccinated mice showed similar neutralization ability against all three spikes. Flow cytometry showed that both B.1.351-LNP-mRNA and B.1.617-LNP-mRNA elicited strong antigen-specific CD8 T cell responses, as well as significant CD4 T cell responses. Single cell transcriptomics of B.1.351-LNP-mRNA and B.1.617-LNP-mRNA vaccinated animals revealed a systematic landscape of immune cell populations, as well as their associated global gene expression status. B.1.351-LNP-mRNA and B.1.617-LNP-mRNA vaccination induced a systemic increase in the reactive CD8 T cell population. Vaccinated animals showed a strong signature of increased expression of transcriptional and translational machineries in B and T cells. BCR-seq and TCR-seq unveiled repertoire diversity and clonality, and respective shifts in vaccinated animals. Animals from both B.1.351-LNP-mRNA and B.1.617-LNP-mRNA groups showed clonal TCR expansion, as evident in both single cell and bulk TCR-seq datasets. These data together provide direct assessment of in vivo immune responses and molecular profiles of vaccination using variant-specific LNP-mRNAs in pre-clinical animal models.

Thus, the vaccines described herein can potently neutralize SARS-CoV-2 variant lineages, such as B.1.1.7, B.1.351, and B.1.617. Also described are pan-coronavirus reactive vaccines that can confer protective immunity against not only the original SARS-CoV-2, but also its variant strains, as well as other pathogenic coronavirus species such as SARS-CoV and/or MERS-CoV. Also described are vaccines in which AAVs and virus like particles (VLPs) are used as the carriers of coronavirus mRNAs and/or proteins encoded therefrom.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### I. Definitions

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

“Introduce,” as used herein, refers to bringing into contact. By “contact” or “contacting” is meant to allow or promote a state of immediate proximity or association between at least two elements. For example, to introduce a composition (e.g., a vector containing a sequence encoding a spike protein or fragment thereof) to a cell is to provide contact between the cell and the composition. The term encompasses penetration of the contacted composition to the interior of the cell by any suitable means, e.g., via transfection, electroporation, transduction, gene gun, nanoparticle delivery, etc.

The term “operably linked” or “operationally linked” refers to functional linkage between a regulatory sequence and a heterologous nucleic acid sequence permitting them to function in their intended manner (e.g., resulting in expression of the latter). The term encompasses positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site.

“Heterologous” is used herein in the context of two more elements having a different, non-native relation, relative position, or structure. The elements can include, but are not limited to, naturally occurring elements from the same or different organisms, chimeric elements, synthetic or engineered elements, etc., provided that the elements are not found in nature in the same relation, relative position, or structure.

“Chimeric” as used in the context of a nucleic acids and proteins describes a non-naturally occurring polynucleotide or polypeptide that is or has a sequence that is made by an artificial combination of two or more otherwise separated segments of sequence. In certain non-limiting embodiments, the sequences combined to form the chimeric nucleic acid or protein are derived from two or more different viral species or strains. This artificial combination is often accomplished by chemical synthesis or by the artificial manipulation of isolated segments of nucleic acids, e.g., by genetic engineering techniques known in the art (e.g., to facilitate addition, substitution, or deletion of a portion of the nucleic acid).

“Isolated” means altered or removed from the natural state. An isolated nucleic acid can exist in substantially purified form, or can exist in a non-native environment such as, for example, a host cell. An “isolated” nucleic acid encompasses a nucleic acid segment or fragment which has been separated from sequences which flank it in a naturally occurring state, e.g., a DNA fragment which has been removed from the sequences which are normally adjacent to the fragment in a genome in which it naturally occurs. The term also applies to nucleic acids which have been substantially purified from other components which naturally accompany the nucleic acid (e.g., RNA or DNA or proteins, which naturally accompany it in the cell). The term therefore includes, for example, a mRNA, or recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. Isolated does not require absolute purity, and can include protein, peptide, nucleic acid, or virus molecules that are at least 50% isolated, such as at least 75%, 80%, 90%, 95%, 98%, 99%, or even 99.9% isolate

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like. “Expression vector” refers to a vector containing a polynucleotide having expression control sequences operatively linked to a nucleotide sequence to be expressed. An expression vector contains sufficient cis-acting elements for expression; other elements for expression can be supplied by the host cell or in an in vitro expression system. Expression vectors include all those known in the art, such as cosmids, plasmids (e.g., naked or contained in liposomes), phagemids, BACs, YACs, and viral vectors (e.g., vectors derived from lentiviruses, retroviruses, adenoviruses, and adeno-associated viruses) that incorporate the recombinant polynucleotide.

The term “mutation” refers to a change in a sequence resulting in an alteration from a given reference sequence. Mutations include a substitution of a residue within a sequence, e.g., a nucleic acid or amino acid sequence, with another residue, or a deletion or insertion of one or more residues within a sequence. In certain non-limiting embodiments, the mutation can be a deletion, insertion, duplication, rearrangement, and/or substitution of at least one deoxyribonucleic acid base such as a purine (adenine and/or guanine) and/or a pyrimidine (thymine, uracil and/or cytosine). In certain non-limiting embodiments, the mutation can be a deletion, insertion, or substitution of at least one amino acid residue in a polypeptide. In certain non-limiting embodiments, mutations are described by identifying the original residue followed by the position of the residue within the sequence and by the identity of the newly substituted residue (e.g., K986P, V987P). Mutations may or may not produce discernible changes in the observable characteristics (phenotype) of a subject.

The term “percent (%) sequence identity” describes the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The % sequence identity of a given nucleic acid or amino acid sequence C to, with, or against a given nucleic acid or amino acid sequence D (which can alternatively be phrased as a given sequence C that has or includes a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

The term “effective amount” means a quantity sufficient to provide a desired pharmacologic and/or physiologic effect.

As used herein, the term “encapsulate” means to enclose, surround or encase.

As used herein, “open reading frame” or “ORF” refers to a sequence which does not contain a stop codon in a given reading frame.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the term “subject” refers to any individual, organism or entity. Typical subjects include animals (e.g., mammals such as mice, rats, rabbits, goats, pigs, chimpanzees, or horses, non-human primates, and humans) and/or plants. The term does not denote a particular age or sex. Thus, adult and newborn subjects, as well as fetuses, whether male or female, are intended to be covered. The subject may be healthy or suffering from or susceptible to a disease, disorder, or condition.

Recitation of ranges of values are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately +/−10%; in other forms the values may range in value either above or below the stated value in a range of approximately +/−5%; in other forms the values may range in value either above or below the stated value in a range of approximately +/−2%; in other forms the values may range in value either above or below the stated value in a range of approximately +/−1%. The preceding ranges are intended to be made clear by context, and no further limitation is implied.

### II. Compositions

Nucleic acids, and compositions and methods of used thereof are disclosed. In particular, compositions, including pharmaceutical compositions, for the preparation and/or formulation of nucleic acids, and which are useful for the generation of vaccines are provided. The compositions are especially useful for delivery of nucleic acids, e.g., a ribonucleic acid (RNA) inside a cell, whether in vitro, in vivo, in situ or ex vivo.

Nucleic acids include any compound and/or substance that constitute a polymer of nucleotides, and hence, can be referred to as polynucleotides. Exemplary nucleic acids or polynucleotides include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs), and hybrids thereof.

A. mRNAs

In certain embodiments, the disclosed nucleic acids are, or include, ribonucleic acids. A non-limiting ribonucleic acid is messenger RNA (mRNA). The term messenger RNA (mRNA) can refer to any ribonucleic acid which directly encodes a polypeptide of interest. Thus, the disclosed mRNAs are capable of being translated to produce one or more encoded polypeptides of interest. In certain non-limiting embodiments, the mRNAs are produced by in vitro transcription.

The mRNAs can be of any suitable length. For example, the length can vary depending upon the size of the encoded polypeptide. mRNA molecules are typically between 200 and 10,000 nucleotides in length. In certain non-limiting embodiments, a mRNA includes about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or 100,000 nucleotides, with or without the poly(A) tail, 5′ UTR, and/or 3′ UTR.

The mRNAs can be codon optimized. For example, the mRNAs can be codon optimized for expression in a eukaryotic cell. The eukaryotic cell can be those of or derived from a particular organism, such as a plant or a mammal, including but not limited to human, or non-human eukaryote or animal or mammal, e.g., mouse, rat, rabbit, dog, livestock, or non-human mammal or primate. Codon-optimization describes gene engineering approaches that use changes of rare codons to synonymous codons that are more frequently used in the cell type of interest with the aim of increasing protein production. In general, codon optimization involves modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA, which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See for example, Nakamura, Y., et al., Nucl. Acids Res., 28:292 (2000). Computer algorithms for codon optimizing a particular sequence for expression in a particular host cell are also available, such as Gene Forge (Aptagen; Jacobus, PA). In certain non-limiting embodiments, one or more codons (e.g., 1, 2, 3, 4, 5, 10, 15, 20, 25, 50, or more, or all codons) in a mRNA corresponds to the most frequently used codon for a particular amino acid.

Typically, the disclosed isolated messenger ribonucleic acids (mRNAs) contain a 5′ untranslated region (UTR), a 3′ UTR, and an open reading frame (also referred to as coding region). In certain non-limiting embodiments, the mRNAs further include a 5′ cap or an analog thereof, a poly(A) tail, one or more modified nucleotides, or a combination thereof. In certain embodiments, the mRNAs include at least a 5′ cap or analog thereof, a 5′ UTR, a 3′ UTR, one or more open reading frames, and a poly(A) tail. In certain embodiments, the mRNAs include at least a 5′ cap or analog thereof, a 5′ UTR, a 3′ UTR, one or more open reading frames, a poly(A) tail, and one or more modified nucleotides.

The mRNA can include different caps or cap analogs (e.g., ARCA). The body of the mRNA can use modified nucleosides. The one or more coding sequences or open reading frames can include various elements such as signal peptides, localization signals (e.g., NLSs), inteins, etc. The structures of the mRNA can be engineered to optimize GC motifs, folding, circularization signals, and/or structured UTR elements.

In certain non-limiting embodiments, the open reading frame encodes a pathogen derived antigen, such as a bacterial, fungal, or viral protein. In certain non-limiting embodiments, the open reading frame encodes all or a portion of one or more proteins from a virus, such as but not limited to a coronavirus, such as SARS-CoV, MERS-CoV, or SARS-CoV-2. In certain embodiments, the open reading frame encodes a coronavirus spike protein sequence, wherein the spike protein sequence includes all or a portion of a coronavirus spike protein. The spike protein can be derived from, for example, SARS-CoV, MERS-CoV, or SARS-CoV-2, including variants thereof.

Thus, a non-limiting mRNA includes a 5′ UTR, a 3′ UTR, and an open reading frame encoding a spike protein sequence derived from SARS-CoV, MERS-CoV, or SARS-CoV-2, including variants thereof, and optionally a 5′ cap or an analog thereof, a poly(A) tail, one or more modified nucleotides, or a combination thereof.

In some certain embodiments, the mRNA is a chimeric (also referred to as hybrid) mRNA. The chimeric mRNA can include one or more (e.g., 1, 2, 3, 4, 5) open reading frames which encode a chimeric (hybrid) spike protein or subunit or other fragment thereof which has sequences from different viral species or variants. For example, a chimeric mRNA can include a 5′ UTR, a 3′ UTR, and one open reading frame which encodes two or more different spike protein sequences (e.g., complete spike proteins or subunits or other fragments thereof) in frame with each other from distinct coronavirus species or variants thereof. As another example, a chimeric mRNA can include a 5′ UTR, a 3′ UTR, and two or more open reading frames, wherein each open reading frame encodes a different spike protein sequence, wherein each spike protein sequence includes a spike protein or subunit or other fragment thereof from the spike protein of a distinct coronavirus species or variant thereof. In certain non-limiting embodiments, the chimeric mRNA does not include a linker or other domain intervening between the two or more open reading frames.

In certain non-limiting embodiments, the mRNA includes a first open reading frame and a second open reading frame. In certain non-limiting embodiments, the first open reading frame encodes a spike protein or subunit or other fragment thereof (e.g., ECD, S1, S2, NTD, RBD) of a coronavirus selected from SARS-CoV, MERS-CoV, SARS-CoV-2 and variants thereof, and the second open reading frame encodes a spike protein or subunit or other fragment thereof (e.g., ECD, S1, S2, NTD, RBD) of a coronavirus selected from SARS-CoV, MERS-CoV, SARS-CoV-2 and variants thereof. In certain non-limiting embodiments, the spike protein or subunit or other fragment thereof encoded by the first open reading frame is from a different viral species or variant from the spike protein or subunit or other fragment thereof encoded by the second open reading frame.

Non-limiting examples of chimeric spike proteins or subunits or other fragment thereof include, a chimeric protein having an S1 subunit from SARS-CoV-2 B.1.351 and an S2 subunit from SARS-CoV-2 B.1.617 (e.g., B.1.617.2 or B.1.617.3), a chimeric protein having an S1 subunit from SARS-CoV and an S2 subunit from SARS-CoV-2 (e.g., native/wildtype, B.1.1.7, B.1.351, B.1.617.2, B.1.617.3), a chimeric protein having an S1 subunit from MERS-CoV and an S2 subunit from SARS-CoV-2 (e.g., native/wildtype, B.1.1.7, B.1.351, B.1.617.2, B.1.617.3), and a chimeric protein having an S1 subunit from MERS-CoV and an S2 subunit from SARS-CoV. Also disclosed are variants of the foregoing in which the viral species or strains from which the S1 and S2 subunits are derived are reversed. In any of the foregoing, the S2 subunit can include one or more mutations, such as proline substitutions that stabilize the spike protein in a prefusion conformation. It is to be understood that while the foregoing examples describe S1 and S2 subunits. In certain non-limiting embodiments, the S1 and/or S2 subunits can be substituted with other spike proteins or subunits or other fragment thereof (e.g., ECD, NTD, RBD).

In certain non-limiting embodiments, each spike protein or subunit or other fragment thereof contains a SPY tag/catcher sequence positioned at the N-terminus or C-terminus. In certain non-limiting embodiments, a chimeric mRNA can include one or more open reading frames encoding a chimeric spike protein in which the mRNA includes a sequence encoding a 2A self-cleaving peptide between adjacent open reading frames or sequences from each distinct viral species or variant. 2A peptides are 18-22 amino acid long viral oligopeptides that mediate cleavage of polypeptides during translation in eukaryotic cells. The mechanism of 2A-mediated self-cleavage is thought to be ribosome skipping the formation of a glycyl-prolyl peptide bond at the C-terminus of the 2A. Suitable 2A self-cleaving peptides include F2A (foot-and-mouth disease virus), E2A (equine rhinitis A virus), P2A (porcine teschovirus-1 2A), and T2A (Thosea asigna virus 2A).

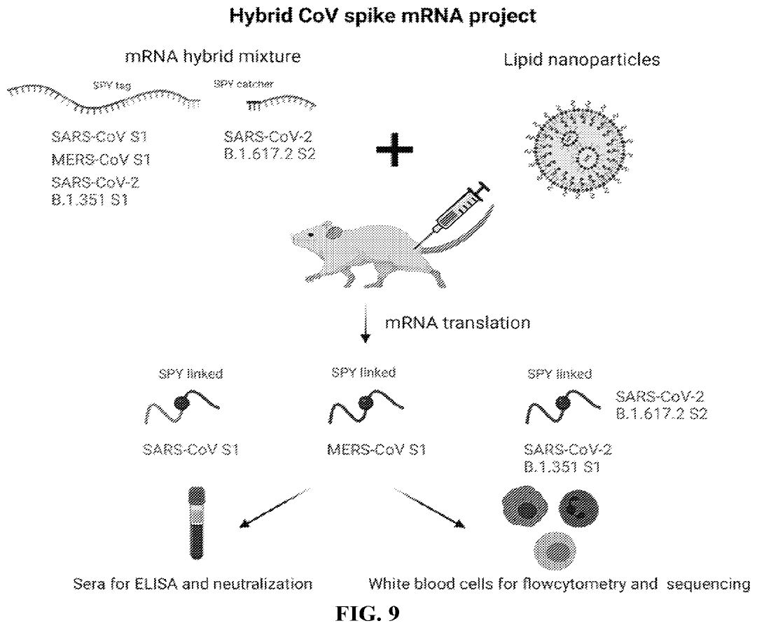

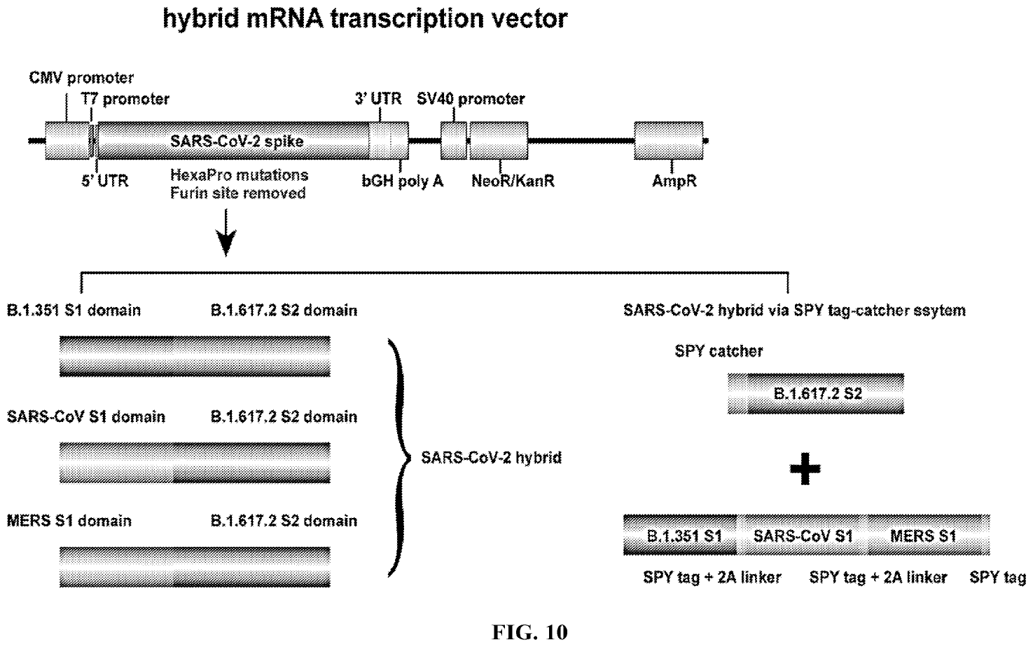

For example, a chimeric mRNA can include one or more open reading frames encoding an S1 subunit of SARS-CoV-2 variant (e.g., SARS-CoV-2 native/wildtype, B.1.1.7, B.1.351, B.1.617.2, B.1.617.3), an S1 subunit of SARS-CoV, and an S1 subunit of MERS-CoV. In certain non-limiting embodiments, the mRNA includes sequences such that each subunit independently includes a SPY tag that, for example, can be fused to the C-terminus of the subunit. In certain non-limiting embodiments, the mRNA further includes a sequence encoding a 2A self-cleaving peptide between each subunit, such that upon translation, separate S1 protein subunits are produced, each having the SPY tag (see, e.g., FIG. 10). In certain non-limiting embodiments, a corresponding mRNA contains an open reading frame encoding an S2 subunit of a coronavirus spike protein in combination with a sequence encoding a SPY catcher at the N-terminus of the spike S2 subunit. Upon translation, the S2 subunit is produced with a SPY catcher at the N-terminus. Upon being brought into close proximity (e.g., by translation in the same cell), a combined protein can be produced by covalent attachment of an S1 subunit with a SPY tag fused to the C-terminus and an S2 subunit with a SPY catcher fused to the N-terminus (see, e.g., FIGS. 9-10).

i. 5′ cap

Typically, the 5′ cap of an mRNA is involved in nuclear export, increasing mRNA stability and binding the mRNA Cap Binding Protein (CBP), which is responsible for mRNA stability in the cell and translation competency through the association of CBP with poly(A) binding protein to form the mature cyclic mRNA species. Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. In certain non-limiting embodiments, the mRNA contains a non-hydrolyzable cap, which can prevent or hinder decapping and thus increase the mRNA half-life. Because cap structure hydrolysis requires cleavage of 5′-ppp-5′ phosphodiester linkages, the 5′ cap can include modified nucleotides to prevent such hydrolysis.

The 5′ cap may be a single nucleotide or a series of nucleotides. For example, the cap may include from 1 to 10, e.g., 2-9, 3-8, 4-7, 1-5, 5-10, or at least 1 or 2, or 10 or fewer nucleotides in length. In certain non-limiting embodiments, the cap is absent.

Cap analogs differ from natural (e.g., endogenous, wild-type or physiological) 5′-caps in their chemical structure, while retaining cap function. Cap analogs may be chemically (e.g., non-enzymatically) or enzymatically synthesized and/or linked to a nucleic acid molecule. For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′ O-Me-m7G(5′)ppp(5′)G). The 3′-O atom of the other, unmodified, guanine becomes linked to the 5′-terminal nucleotide of the capped nucleic acid molecule (e.g., mRNA). The N7- and 3′-O-methylated guanine provides the terminal moiety of the capped nucleic acid molecule. Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

In certain non-limiting embodiments, a 5′ cap may include endogenous caps or cap analogs. For example, a 5′ cap may include a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

Suitable 5′ caps or analogs that can be included in the mRNAs are known in the art and include, without limitation, 7mG(5′)ppp(5′)N,pN2p (cap 0), 7mG(5′)ppp(5′)NlmpNp (cap 1), 7mG(5′)-ppp(5′)NlmpN2mp (cap 2), ARCA, beta-S-ARCA, m7G, mCAP, inosine, N1-methyl-guanosine, 2′-fluoro-guanosine, 7-deaza-guanosine, 8-oxo-guanosine, 2-amino-guanosine, LNA-guanosine, tri-methylgranosine (TMG), nicotinamide adenine dinucleotide (NAD), cap AG, cap AU, cap GG, and 2-azido-guanosine.

ii. Untranslated Regions

Untranslated regions (UTRs) are regions of a gene that are transcribed, but not translated. Generally, the 5′UTR starts at the transcription start site and continues to the start codon but does not include the start codon; whereas the 3′UTR starts immediately following the stop codon and continues until the transcriptional termination signal. 5′ UTRs can harbor specific regions, like Kozak sequences which are be involved in the initiation of translation by the ribosome. 5′ UTRs also have been known to form secondary structures which are involved in elongation factor binding. The UTRs can have important regulatory effects on an associated mRNA, for example impacting stability and/or translation of the mRNA. Generally, translational efficiency (including activation or inhibition of translation) of mRNAs can be controlled by the UTRs. In certain non-limiting embodiments, the regulatory features of a UTR can be incorporated into the disclosed mRNAs, to enhance the stability of the molecule. In certain non-limiting embodiments, the mRNAs are engineered to contain the UTRs found in abundantly expressed genes to enhance the enhance the stability and protein production from the mRNA. For example, introduction of 5′ UTR of liver-expressed mRNA, such as albumin, serum amyloid A, Apolipoprotein A/B/E, transferrin, alpha fetoprotein, erythropoietin, or Factor VIII, could be used to enhance expression of an mRNA. Likewise, use of 5′ UTR from other tissue-specific mRNA to improve expression in that tissue is possible for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D).

iii. Poly(A) Tails

During RNA processing, a long chain of adenine nucleotides, referred to as the poly(A) tail, may be added to a polynucleotide such as an mRNA in order to increase stability. Immediately after transcription, the 3′ end of the transcript may be cleaved to free a 3′ hydroxyl. Then, poly-A polymerase adds a chain of adenine nucleotides to the RNA. The process, called polyadenylation, adds a poly(A) tail that can be between, for example, approximately 100 and 250 residues long.

In certain non-limiting embodiments, the poly(A) tail includes about 10-100, about 100-300, about 100-250, or about 100-200 adenines. In certain non-limiting embodiments, the poly(A) tail contains about 10, 15, 20, 25, 30, 35, 40, 45, 50, 55, 60, 65, 70, 75, 80, 85, 90, 95, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, or 3,000 nucleotides.

iv. Modified Nucleotides

The mRNA can be modified or unmodified. The mRNA can be modified for example, to optimize translation, and/or to confer increased stability and/or expression. In certain non-limiting embodiments, a mRNA or other modified polynucleotide may exhibit reduced degradation when introduced to a cell as compared to a corresponding unmodified polynucleotide.

The modified mRNA or other modified polynucleotide can incorporate a number of chemical changes to the nucleotides, including changes to the nucleobase, the ribose or deoxyribose sugar, and/or the phosphodiester linkage. One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain forms, modifications (e.g., one or more modifications) are present in each of the sugar and the internucleotide linkage.

Backbone phosphate groups can be modified by replacing one or more of the oxygen atoms with a different substituent. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates). Phosphorothioate DNA and RNA have increased nuclease resistance, and subsequently, a longer half-life in a cellular environment.

In certain embodiments, the mRNA or other polynucleotide includes one or more modified nucleotides. For example, the mRNA or other polynucleotide can include one or more modified guanine-, adenine-, cytosine-, thymidine-, and/or uridine-containing nucleotides. Suitable modified nucleotides/nucleosides include, without limitation, pseudouridine, N1-methyl-pseudouridine, N1-Methylpseudouridine-5′-Triphosphate-(N-1081), 1-ethylpseudouridine, 2-thiouridine, 4′-thiouridine, 5-methoxyuridine, 5-methoxyuridine, N6-methyladenosine, 5-methylcytosine, 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine (e.g., 5-iodo-cytidine), 5-hydroxymethyl-cytidine, 1-methyl-pseudoisocytidine, 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 1-methyl-adenosine, 2-methyl-adenine, N6-methyl-adenosine, 2-methylthio-N6-methyl-adenosine, N6-isopentenyl-adenosine, inosine, 1-methyl-inosine, wyosine, methylwyosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, and 6-methoxy-guanosine. For example, N1-Methylpseudouridine-5′-Triphosphate-(N-1081) can be utilized during in vitro transcription so that it is incorporated into the mRNA.

In certain non-limiting embodiments, all of the instances of a given nucleotide (e.g., every G, every A, every C, every T, or every U) are modified. In certain non-limiting embodiments, a fraction of the instances of a given nucleotide are modified. For example, about 0.1%, 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of a given nucleotide can be modified.

As a non-limiting example, the nucleotide uridine may be substituted with a modified nucleotide described herein, such as N1-methyl-pseudouridine. In certain non-limiting embodiments, the uridine in the mRNA is partially substituted. For example, about 0.1%, 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the uridine in a given mRNA may be substituted with a modified nucleotide, such as N1-methyl-pseudouridine. For example, in some certain embodiments, about 50% of uridine is substituted with a modified nucleotide, such as N1-methyl-pseudouridine.

### V. Open Reading Frame Encoded Polypeptides

The mRNAs contain sequences that encode polypeptides of interest. For example, an mRNA can contain one or more open reading frames, each of which encodes one or more polypeptides. Typically, the open reading frame encodes an antigen (e.g., protein or peptide) from a pathogenic microorganism, such as bacteria, fungi, protozoa, or virus. In certain non-limiting embodiments, the open reading frame encodes one or more proteins from a virus, or an immune-response inducing fragment or variant thereof.

### Coronavirus Polypeptides

In certain non-limiting embodiments, the mRNA includes an open reading frame that encodes one or more immunogenic proteins or subunits or other fragments thereof from a coronavirus. Coronaviruses are large, enveloped, positive-stranded RNA viruses (Li, “Structure, Function, and Evolution of Coronavirus Spike Proteins,” Annual Review of Virology, 3:1, 237-261 (2016)). Coronaviruses are phylogenetically divided into four genera (α, β, γ, δ), with betacoronaviruses further subdivided into four lineages (A, B, C, D). Coronaviruses infect a wide range of avian and mammalian species, including humans.

Coronaviruses have the largest genome among RNA viruses, typically ranging from 26 to 32 kb. The genome is packed inside a helical capsid formed by the nucleocapsid protein (N) and further surrounded by an envelope. Associated with the viral envelope are at least three structural proteins: the membrane protein (M) and the envelope protein (E) are involved in virus assembly, whereas the spike protein(S) mediates virus entry into host cells. Some coronaviruses also encode an envelope-associated hemagglutinin-esterase protein (HE). Thus, In certain non-limiting embodiments, the mRNA open reading frame encodes a coronavirus M, S, and/or E protein, or an immune response-inducing subunit, fragment, or variant derived therefrom.

Among these structural proteins, the spike protein forms large protrusions from the virus surface, giving coronaviruses the appearance of having crowns. In addition to mediating virus entry, the spike is an important determinant of viral host range and tissue tropism and a major inducer of host immune responses. The coronavirus spike contains three segments: a large ectodomain, a single-pass transmembrane anchor, and a short intracellular tail. The ectodomain includes a receptor-binding S1 subunit and a membrane-fusion S2 subunit. The spike protein is initially synthesized as a precursor protein. Individual precursor S polypeptides form a homotrimer and undergo glycosylation within the Golgi apparatus as well as processing to remove the signal peptide, and cleavage by a cellular protease to generate separate S1 and S2 polypeptide chains, which remain associated as S1/S2 protomers within the homotrimer (which is therefore a trimer of heterodimers). The S1 subunit contains the N-terminal domain (NTD) and receptor-binding domain (RBD) which mediates virus attachment to its host receptor. The S2 subunit contains fusion protein machinery, such as the fusion peptide, two heptad-repeat sequences (HR1 and HR2) and a central helix typical of fusion glycoproteins, a transmembrane domain, and the cytosolic tail domain. The spike is a clove-shaped trimer with three S1 heads and a trimeric S2 stalk. During virus entry, S1 binds to a receptor on the host cell surface for viral attachment, and S2 fuses the host and viral membranes, allowing viral genomes to enter host cells.

Thus, in certain embodiments, the mRNA open reading frame encodes a spike protein sequence, wherein the spike protein sequence includes all or a portion of a coronavirus spike protein, such as a coronavirus species or variant disclosed herein. For example, the open reading frame can encode a coronavirus spike protein or subunit or other fragment thereof (e.g., ECD, S1, S2, NTD and/or RBD), optionally wherein the coronavirus spike protein or subunit or other fragment thereof is derived from one of the species or exemplary viruses mentioned below.

Non-limiting examples of betacoronaviruses include Middle East respiratory syndrome coronavirus (MERS-CoV), Severe Acute Respiratory Syndrome coronavirus (SARS-CoV), SARS-CoV-2, Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), RaTG13 bat coronavirus, and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronavirus is the Swine Delta Coronavirus (SDCV).

Representative coronaviruses include SARSr-CoV BtKY72, SARS-CoV, SARS-CoV-2, SARSr-CoV RaTG13, SARS-CoV PC4-227, Bat-Hp-BetaCovC, Ro-BatCoV GCCDC1, Ro-BatCoV HKU9, Pi-BatCoV HKU5, Ty-BatCoV HKU4, MERS-CoV, EriCoV, MHV, HCoV HKU1, ChRCoV HKU24, ChRCovC HKU24, MrufCoV 2JL14, HCoV NL63, HCoV 229E, and HCoV OC43. See, e.g., Coronaviridae Study Group of the International Committee on Taxonomy of Viruses, Nat Microbiol 2020. DOI: 10.1038/s41564-020-0695-z), which is specifically incorporated by reference in its entirety.

In certain embodiments, the coronavirus is selected from SARS-CoV, MERS-CoV, and SARS-CoV-2. SARS-CoV2 is a new β-coronavirus after the previously identified SARS-CoV and MERS-CoV which led to pulmonary failure and potentially fatal respiratory tract infection. SARS-CoV-2 causes Coronavirus disease 2019 (COVID-2019). SARS-CoV-2 spike protein facilitates entry of the virus into host cells by binding to angiotensin-converting enzyme 2 (ACE2) expressed in lower respiratory tract cells. The spike is cleaved by the host cell furin-like protease into the S1 and S2 subunits. Table 1 describes the typical architecture of a wildtype SARS-CoV-2 spike protein (see also UniProtKB ID NO. P0DTC2 (SPIKE_SARS2)). In certain embodiments, the open reading frame encodes a coronavirus spike protein or subunit or other fragment thereof (e.g., ECD, S1, S2, NTD and/or RBD), from SARS-CoV, MERS-CoV, or SARS-CoV-2.

TABLE 1
Features of wildtype SARS-CoV-2 Spike Protein

| Feature key | Position(s) | Description |
|---|---|---|
| Signal peptide | 1-12 | |
| Chain | 13-1273 | Spike glycoprotein |
| PRO_0000449646 | | |
| Chain | 13-685 | Spike protein S1 |
| PRO_0000449647 | | |
| Chain | 686-1273 | Spike protein S2 |
| PRO_0000449648 | | |
| Chain | 816-1273 | Spike protein S2′ |
| PRO_0000449649 | | |

Various strains/variants of the foregoing viruses are known and include, without limitation, SARS-CoV-2 B.1.1.7 (Alpha variant), SARS-CoV-2 B.1.351 (Beta variant, including B.1.351.2, and B.1.351.3), SARS-CoV-2 B.1.617 (including SARS-CoV-2 B.1.617.1 (Kapa), SARS-CoV-2 B.1.617.2 (Delta), and SARS-CoV-2 B.1.617.3), gamma variant (including P.1, P.1.1, and P.1.2), Epsilon variant B.1.427 and B.1.429, Eta variant B.1.525, Iota variant B.1.526, Zeta variant P.2, Mu variant B.1.621, B.1.621.1, B.1.1.529/BA.1 (Omicron variant), BA.5, BA.2, BA.2.12.1, and BA.4/5. and other emerging global or regional variants. Thus, in certain embodiments, the open reading frame encodes a coronavirus spike protein or subunit or other fragment thereof (e.g., ECD, S1, S2, NTD and/or RBD) from any of the foregoing strains/variants.

Exemplary gene, protein, and genomic sequences of the foregoing coronavirus species and strains are known in the art. See, for example, the sequences and accession numbers provided in, Coronaviridae Study Group of the International Committee on Taxonomy of Viruses, Nat Microbiol 2020. (DOI: 10.1038/s41564-020-0695-z), which is hereby incorporated by reference in its entirety. For example, GenBank Accession No. MN908947.3, which is specifically incorporated by reference herein in its entirety, provides a (DNA) genomic sequence for SARS-CoV-2 (Severe acute respiratory syndrome coronavirus 2 isolate Wuhan-Hu-1, complete genome). GenBank Accession No. MN985325.1, which is specifically incorporated by reference herein in its entirety, also provides a genomic DNA sequence for SARS-CoV-2 (Severe acute respiratory syndrome coronavirus 2 isolate 2019-nCoV/USA-WA1/2020, complete genome). It will be appreciated that the sequences are provided as DNA sequences, but the viral genome itself will typically have the corresponding RNA sequences. Thus, the corresponding RNA sequences are also expressly provided herein.

An exemplary cDNA sequence that encodes a
SARS-CoV-2 wildtype (Wuhan-Hu-1) spike
protein is:
(SEQ ID NO: 1)
ATGTTTGTGTTCCTGGTGCTGCTGCCACTGGTGTCCAGCCA
GTGTGTGAACCTGACCACCAGGACCCAACTTCCTCCTGCCT
ACACCAACTCCTTCACCAGGGGAGTCTACTACCCTGACAAG
GTGTTCAGGTCCTCTGTGCTGCACAGCACCCAGGACCTGTT
CCTGCCATTCTTCAGCAATGTGACCTGGTTCCATGCCATCC
ATGTGTCTGGCACCAATGGCACCAAGAGGTTTGACAACCCT
GTGCTGCCATTCAATGATGGAGTCTACTTTGCCAGCACAGA
GAAGAGCAACATCATCAGGGGCTGGATTTTTGGCACCACCC
TGGACAGCAAGACCCAGTCCCTGCTGATTGTGAACAATGCC
ACCAATGTGGTGATTAAGGTGTGTGAGTTCCAGTTCTGTAA
TGACCCATTCCTGGGAGTCTACTACCACAAGAACAACAAGT
CCTGGATGGAGTCTGAGTTCAGGGTCTACTCCTCTGCCAAC
AACTGTACCTTTGAATATGTGAGCCAACCATTCCTGATGGA
CTTGGAGGGCAAGCAGGGCAACTTCAAGAACCTGAGGGAGT
TTGTGTTCAAGAACATTGATGGCTACTTCAAGATTTACAGC
AAACACACACCAATCAACCTGGTGAGGGACCTGCCACAGGG
CTTCTCTGCCTTGGAACCACTGGTGGACCTGCCAATTGGCA
TCAACATCACCAGGTTCCAGACCCTGCTGGCTCTGCACAGG
TCCTACCTGACACCTGGAGACTCCTCCTCTGGCTGGACAGC
AGGAGCAGCAGCCTACTATGTGGGCTACCTCCAACCAAGGA
CCTTCCTGCTGAAATACAATGAGAATGGCACCATCACAGAT
GCTGTGGACTGTGCCCTGGACCCACTGTCTGAGACCAAGTG
TACCCTGAAATCCTTCACAGTGGAGAAGGGCATCTACCAGA
CCAGCAACTTCAGGGTCCAACCAACAGAGAGCATTGTGAGG
TTTCCAAACATCACCAACCTGTGTCCATTTGGAGAGGTGTT
CAATGCCACCAGGTTTGCCTCTGTCTATGCCTGGAACAGGA
AGAGGATTAGCAACTGTGTGGCTGACTACTCTGTGCTCTAC
AACTCTGCCTCCTTCAGCACCTTCAAGTGTTATGGAGTGAG
CCCAACCAAACTGAATGACCTGTGTTTCACCAATGTCTATG
CTGACTCCTTTGTGATTAGGGGAGATGAGGTGAGACAGATT
GCCCCTGGACAAACAGGCAAGATTGCTGACTACAACTACAA
ACTGCCTGATGACTTCACAGGCTGTGTGATTGCCTGGAACA
GCAACAACCTGGACAGCAAGGTGGGAGGCAACTACAACTAC
CTCTACAGACTGTTCAGGAAGAGCAACCTGAAACCATTTGA
GAGGGACATCAGCACAGAGATTTACCAGGCTGGCAGCACAC
CATGTAATGGAGTGGAGGGCTTCAACTGTTACTTTCCACTC
CAATCCTATGGCTTCCAACCAACCAATGGAGTGGGCTACCA
ACCATACAGGGTGGTGGTGCTGTCCTTTGAACTGCTCCATG
CCCCTGCCACAGTGTGTGGACCAAAGAAGAGCACCAACCTG
GTGAAGAACAAGTGTGTGAACTTCAACTTCAATGGACTGAC
AGGCACAGGAGTGCTGACAGAGAGCAACAAGAAGTTCCTGC
CATTCCAACAGTTTGGCAGGGACATTGCTGACACCACAGAT
GCTGTGAGGGACCCACAGACCTTGGAGATTCTGGACATCAC
ACCATGTTCCTTTGGAGGAGTGTCTGTGATTACACCTGGCA
CCAACACCAGCAACCAGGTGGCTGTGCTCTACCAGGATGTG
AACTGTACTGAGGTGCCTGTGGCTATCCATGCTGACCAACT
TACACCAACCTGGAGGGTCTACAGCACAGGCAGCAATGTGT
TCCAGACCAGGGCTGGCTGTCTGATTGGAGCAGAGCATGTG
AACAACTCCTATGAGTGTGACATCCCAATTGGAGCAGGCAT
CTGTGCCTCCTACCAGACCCAGACCAACAGCCCAAGGAGGG
CAAGGTCTGTGGCAAGCCAGAGCATCATTGCCTACACAATG
AGTCTGGGAGCAGAGAACTCTGTGGCTTACAGCAACAACAG
CATTGCCATCCCAACCAACTTCACCATCTCTGTGACCACAG
AGATTCTGCCTGTGAGTATGACCAAGACCTCTGTGGACTGT
ACAATGTATATCTGTGGAGACAGCACAGAGTGTAGCAACCT
GCTGCTCCAATATGGCTCCTTCTGTACCCAACTTAACAGGG
CTCTGACAGGCATTGCTGTGGAACAGGACAAGAACACCCAG
GAGGTGTTTGCCCAGGTGAAGCAGATTTACAAGACACCTCC
AATCAAGGACTTTGGAGGCTTCAACTTCAGCCAGATTCTGC
CTGACCCAAGCAAGCCAAGCAAGAGGTCCTTCATTGAGGAC
CTGCTGTTCAACAAGGTGACCCTGGCTGATGCTGGCTTCAT
CAAGCAATATGGAGACTGTCTGGGAGACATTGCTGCCAGGG
ACCTGATTTGTGCCCAGAAGTTCAATGGACTGACAGTGCTG
CCTCCACTGCTGACAGATGAGATGATTGCCCAATACACCTC
TGCCCTGCTGGCTGGCACCATCACCTCTGGCTGGACCTTTG
GAGCAGGAGCAGCCCTCCAAATCCCATTTGCTATGCAGATG
GCTTACAGGTTCAATGGCATTGGAGTGACCCAGAATGTGCT
CTATGAGAACCAGAAACTGATTGCCAACCAGTTCAACTCTG
CCATTGGCAAGATTCAGGACTCCCTGTCCAGCACAGCCTCT
GCCCTGGGCAAACTCCAAGATGTGGTGAACCAGAATGCCCA
GGCTCTGAACACCCTGGTGAAGCAACTTTCCAGCAACTTTG
GAGCCATCTCCTCTGTGCTGAATGACATCCTGAGCAGACTG
GACAAGGTGGAGGCTGAGGTCCAGATTGACAGACTGATTAC
AGGCAGACTCCAATCCCTCCAAACCTATGTGACCCAACAAC
TTATCAGGGCTGCTGAGATTAGGGCATCTGCCAACCTGGCT
GCCACCAAGATGAGTGAGTGTGTGCTGGGACAAAGCAAGAG
GGTGGACTTCTGTGGCAAGGGCTACCACCTGATGAGTTTTC
CACAGTCTGCCCCTCATGGAGTGGTGTTCCTGCATGTGACC
TATGTGCCTGCCCAGGAGAAGAACTTCACCACAGCCCCTGC
CATCTGCCATGATGGCAAGGCTCACTTTCCAAGGGAGGGAG
TGTTTGTGAGCAATGGCACCCACTGGTTTGTGACCCAGAGG
AACTTCTATGAACCACAGATTATCACCACAGACAACACCTT
TGTGTCTGGCAACTGTGATGTGGTGATTGGCATTGTGAACA
ACACAGTCTATGACCCACTCCAACCTGAACTGGACTCCTTC
AAGGAGGAACTGGACAAATACTTCAAGAACCACACCAGCCC
TGATGTGGACCTGGGAGACATCTCTGGCATCAATGCCTCTG
TGGTGAACATCCAGAAGGAGATTGACAGACTGAATGAGGTG
GCTAAGAACCTGAATGAGTCCCTGATTGACCTCCAAGAACT
GGGCAAATATGAACAATACATCAAGTGGCCATGGTACATCT
GGCTGGGCTTCATTGCTGGACTGATTGCCATTGTGATGGTG
ACCATAATGCTGTGTTGTATGACCTCCTGTTGTTCCTGTCT
GAAAGGCTGTTGTTCCTGTGGCTCCTGTTGTAAGTTTGATG
AGGATGACTCTGAACCTGTGCTGAAAGGAGTGAAACTGCAC
TACACCTGA,

wherein nucleotides 1-2445 constitute the S1 subunit, nucleotides 2446-3822 constitute the S2 subunit, and nucleotides 2044-2055 (bolded) constitute the furin cleavage site. It will be appreciated that though the cDNA sequence is provided, the corresponding mRNA sequence encoding the spike protein is also expressly provided herein.

An exemplary amino acid sequence of a
SARS-CoV-2 wildtype (Wuhan-Hu-1) spike
protein is:
(SEQ ID NO: 2)
MFVFLVLLPLVSSQCVNLTTRTQLPPAYTNSFTRGVYYPD
KVFRSSVLHSTQDLFLPFFSNVTWFHAIHVSGTNGTKRFD
NPVLPFNDGVYFASTEKSNIIRGWIFGTTLDSKTQSLLIV
NNATNVVIKVCEFQFCNDPFLGVYYHKNNKSWMESEFRVY
SSANNCTFEYVSQPFLMDLEGKQGNFKNLREFVFKNIDGY
FKIYSKHTPINLVRDLPQGFSALEPLVDLPIGINITRFQT
LLALHRSYLTPGDSSSGWTAGAAAYYVGYLQPRTFLLKYN
ENGTITDAVDCALDPLSETKCTLKSFTVEKGIYQTSNFRV
QPTESIVRFPNITNLCPFGEVFNATRFASVYAWNRKRISN
CVADYSVLYNSASFSTFKCYGVSPTKLNDLCFTNVYADSF
VIRGDEVRQIAPGQTGKIADYNYKLPDDFTGCVIAWNSNN
LDSKVGGNYNYLYRLFRKSNLKPFERDISTEIYQAGSTPC
NGVEGFNCYFPLQSYGFQPTNGVGYQPYRVVVLSFELLHA
PATVCGPKKSTNLVKNKCVNFNFNGLTGTGVLTESNKKFL
PFQQFGRDIADTTDAVRDPQTLEILDITPCSFGGVSVITP
GTNTSNQVAVLYQDVNCTEVPVAIHADQLTPTWRVYSTGS
NVFQTRAGCLIGAEHVNNSYECDIPIGAGICASYQTQTNS
PRRARSVASQSIIAYTMSLGAENSVAYSNNSIAIPTNFTI
SVTTEILPVSMTKTSVDCTMYICGDSTECSNLLLQYGSFC
TQLNRALTGIAVEQDKNTQEVFAQVKQIYKTPPIKDFGGF
NFSQILPDPSKPSKRSFIEDLLFNKVTLADAGFIKQYGDC
LGDIAARDLICAQKFNGLTVLPPLLTDEMIAQYTSALLAG
TITSGWTFGAGAALQIPFAMQMAYRFNGIGVTQNVLYENQ
KLIANQFNSAIGKIQDSLSSTASALGKLQDVVNQNAQALN
TLVKQLSSNFGAISSVLNDILSRLDKVEAEVQIDRLITGR
LQSLQTYVTQQLIRAAEIRASANLAATKMSECVLGQSKRV
DFCGKGYHLMSFPQSAPHGVVFLHVTYVPAQEKNFTTAPA
ICHDGKAHFPREGVFVSNGTHWFVTQRNFYEPQIITTDNT
FVSGNCDVVIGIVNNTVYDPLQPELDSFKEELDKYFKNHT
SPDVDLGDISGINASVVNIQKEIDRLNEVAKNLNESLIDL
QELGKYEQYIKWPWYIWLGFIAGLIAIVMVTIMLCCMTSC
CSCLKGCCSCGSCCKFDEDDSEPVLKGVKLHYT,

wherein residues 13-685 constitute the S1 subunit, residues 686-1273 constitute the S2 subunit, and residues 682-685 (bolded) constitute the furin cleavage site.

An exemplary amino acid sequence of a MERS-CoV
wildtype spike protein is:
(SEQ ID NO: 3; UniProt ID: K9N5Q8)
MIHSVFLLMFLLTPTESYVDVGPDSVKSACIEVDIQQTFFDKTWPR
PIDVSKADGIIYPQGRTYSNITITYQGLFPYQGDHGDMYVYSAGHA
TGTTPQKLFVANYSQDVKQFANGFVVRIGAAANSTGTVIISPSTSA
TIRKIYPAFMLGSSVGNFSDGKMGRFFNHTLVLLPDGCGTLLRAFY
CILEPRSGNHCPAGNSYTSFATYHTPATDCSDGNYNRNASLNSFKE
YFNLRNCTFMYTYNITEDEILEWFGITQTAQGVHLFSSRYVDLYGG
NMFQFATLPVYDTIKYYSIIPHSIRSIQSDRKAWAAFYVYKLQPLT
FLLDFSVDGYIRRAIDCGFNDLSQLHCSYESFDVESGVYSVSSFEA
KPSGSVVEQAEGVECDFSPLLSGTPPQVYNFKRLVFTNCNYNLTKL
LSLFSVNDFTCSQISPAAIASNCYSSLILDYFSYPLSMKSDLSVSS
AGPISQFNYKQSFSNPTCLILATVPHNLTTITKPLKYSYINKCSRF
LSDDRTEVPQLVNANQYSPCVSIVPSTVWEDGDYYRKQLSPLEGGG
WLVASGSTVAMTEQLQMGFGITVQYGTDTNSVCPKLEFANDTKIAS
QLGNCVEYSLYGVSGRGVFQNCTAVGVRQQRFVYDAYQNLVGYYSD
DGNYYCLRACVSVPVSVIYDKETKTHATLFGSVACEHISSTMSQYS
RSTRSMLKRRDSTYGPLQTPVGCVLGLVNSSLFVEDCKLPLGQSLC
ALPDTPSTLTPRSVRSVPGEMRLASIAFNHPIQVDQLNSSYFKLSI
PTNFSFGVTQEYIQTTIQKVTVDCKQYVCNGFQKCEQLLREYGQFC
SKINQALHGANLRQDDSVRNLFASVKSSQSSPIIPGFGGDFNLTLL
EPVSISTGSRSARSAIEDLLFDKVTIADPGYMQGYDDCMQQGPASA
RDLICAQYVAGYKVLPPLMDVNMEAAYTSSLLGSIAGVGWTAGLSS
FAAIPFAQSIFYRLNGVGITQQVLSENQKLIANKFNQALGAMQTGF
TTTNEAFHKVQDAVNNNAQALSKLASELSNTFGAISASIGDIIQRL
DVLEQDAQIDRLINGRLTTLNAFVAQQLVRSESAALSAQLAKDKVN
ECVKAQSKRSGFCGQGTHIVSFVVNAPNGLYFMHVGYYPSNHIEVV
SAYGLCDAANPTNCIAPVNGYFIKTNNTRIVDEWSYTGSSFYAPEP
ITSLNTKYVAPQVTYQNISTNLPPPLLGNSTGIDFQDELDEFFKNV
STSIPNFGSLTQINTTLLDLTYEMLSLQQVVKALNESYIDLKELGN
YTYYNKWPWYIWLGFIAGLVALALCVFFILCCTGCGTNCMGKLKCN
RCCDRYEEYDLEPHKVHVH.
An exemplary amino acid sequence of a SARS-CoV
wildtype spike protein is:
(SEQ ID NO: 4; UniProt ID: P59594)
MFIFLLFLTLTSGSDLDRCTTFDDVQAPNYTQHTSSMRGVYYPDEI
FRSDTLYLTQDLFLPFYSNVTGFHTINHTFGNPVIPFKDGIYFAAT
EKSNVVRGWVFGSTMNNKSQSVIIINNSTNVVIRACNFELCDNPFF
AVSKPMGTQTHTMIFDNAFNCTFEYISDAFSLDVSEKSGNFKHLRE
FVFKNKDGFLYVYKGYQPIDVVRDLPSGFNTLKPIFKLPLGINITN
FRAILTAFSPAQDIWGTSAAAYFVGYLKPTTFMLKYDENGTITDAV
DCSQNPLAELKCSVKSFEIDKGIYQTSNFRVVPSGDVVRFPNITNL
CPFGEVFNATKFPSVYAWERKKISNCVADYSVLYNSTFFSTFKCYG
VSATKLNDLCFSNVYADSFVVKGDDVRQIAPGQTGVIADYNYKLPD
DFMGCVLAWNTRNIDATSTGNYNYKYRYLRHGKLRPFERDISNVPF
SPDGKPCTPPALNCYWPLNDYGFYTTTGIGYQPYRVVVLSFELLNA
PATVCGPKLSTDLIKNQCVNFNFNGLTGTGVLTPSSKRFQPFQQFG
RDVSDFTDSVRDPKTSEILDISPCSFGGVSVITPGTNASSEVAVLY
QDVNCTDVSTAIHADQLTPAWRIYSTGNNVFQTQAGCLIGAEHVDT
SYECDIPIGAGICASYHTVSLLRSTSQKSIVAYTMSLGADSSIAYS
NNTIAIPTNFSISITTEVMPVSMAKTSVDCNMYICGDSTECANLLL
QYGSFCTQLNRALSGIAAEQDRNTREVFAQVKQMYKTPTLKYFGGF
NFSQILPDPLKPTKRSFIEDLLFNKVTLADAGFMKQYGECLGDINA
RDLICAQKFNGLTVLPPLLTDDMIAAYTAALVSGTATAGWTFGAGA
ALQIPFAMQMAYRFNGIGVTQNVLYENQKQIANQFNKAISQIQESL
TTTSTALGKLQDVVNQNAQALNTLVKQLSSNFGAISSVLNDILSRL
DKVEAEVQIDRLITGRLQSLQTYVTQQLIRAAEIRASANLAATKMS
ECVLGQSKRVDFCGKGYHLMSFPQAAPHGVVFLHVTYVPSQERNFT
TAPAICHEGKAYFPREGVFVFNGTSWFITQRNFFSPQIITTDNTFV
SGNCDVVIGIINNTVYDPLQPELDSFKEELDKYFKNHTSPDVDLGD
ISGINASVVNIQKEIDRLNEVAKNLNESLIDLQELGKYEQYIKWPW
YVWLGFIAGLIAIVMVTILLCCMTSCCSCLKGACSCGSCCKFDEDD
SEPVLKGVKLHYT.
An exemplary amino acid sequence of a
SARS-CoV-2 B.1.351 spike protein con-
taining six stabilizing proline substi-
tutions (shown in bold underline) is:
(SEQ ID NO: 5)
MFVFLVLLPLVSSQCVNFTTRTQLPPAYTNSFTRGVYYPDKVFRSS
VLHSTQDLFLPFFSNVTWFHAIHVSGTNGTKRFANPVLPFNDGVYF
ASTEKSNIIRGWIFGTTLDSKTQSLLIVNNATNVVIKVCEFQFCND
PFLGVYYHKNNKSWMESEFRVYSSANNCTFEYVSQPFLMDLEGKQG
NFKNLREFVFKNIDGYFKIYSKHTPINLVRGLPQGFSALEPLVDLP
IGINITRFQTLLALHISYLTPGDSSSGWTAGAAAYYVGYLQPRTFL
LKYNENGTITDAVDCALDPLSETKCTLKSFTVEKGIYQTSNFRVQP
TESIVRFPNITNLCPFGEVFNATRFASVYAWNRKRISNCVADYSVL
YNSASFSTFKCYGVSPTKLNDLCFTNVYADSFVIRGDEVRQIAPGQ
TGNIADYNYKLPDDFTGCVIAWNSNNLDSKVGGNYNYLYRLFRKSN
LKPFERDISTEIYQAGSTPCNGVKGFNCYFPLQSYGFQPTYGVGYQ
PYRVVVLSFELLHAPATVCGPKKSTNLVKNKCVNFNFNGLTGTGVL
TESNKKFLPFQQFGRDIADTTDAVRDPQTLEILDITPCSFGGVSVI
TPGTNTSNQVAVLYQDVNCTEVPVAIHADQLTPTWRVYSTGSNVFQ
TRAGCLIGAEHVNNSYECDIPIGAGICASYQTQTNSPGSASSVASQ
SIIAYTMSLGVENSVAYSNNSIAIPTNFTISVTTEILPVSMTKTSV
DCTMYICGDSTECSNLLLQYGSFCTQLNRALTGIAVEQDKNTQEVF
AQVKQIYKTPPIKDFGGFNFSQILPDPSKPSKRSPIEDLLFNKVTL
ADAGFIKQYGDCLGDIAARDLICAQKFNGLTVLPPLLTDEMIAQYT
SALLAGTITSGWTFGAGPALQIPFPMQMAYRFNGIGVTQNVLYENQ
KLIANQFNSAIGKIQDSLSSTPSALGKLQDVVNQNAQALNTLVKQL
SSNFGAISSVLNDILSRLDPPEAEVQIDRLITGRLQSLQTYVTQQL
IRAAEIRASANLAATKMSECVLGQSKRVDFCGKGYHLMSFPQSAPH
GVVFLHVTYVPAQEKNFTTAPAICHDGKAHFPREGVFVSNGTHWFV
TQRNFYEPQIITTDNTFVSGNCDVVIGIVNNTVYDPLQPELDSFKE
ELDKYFKNHTSPDVDLGDISGINASVVNIQKEIDRLNEVAKNLNES
LIDLQELGKYEQYIKWPWYIWLGFIAGLIAIVMVTIMLCCMTSCCS
CLKGCCSCGSCCKFDEDDSEPVLKGVKLHYT,

wherein residues 13-685 constitute the S1 subunit, residues 686-1273 constitute the S2 subunit, and residues 682-685 (bolded) constitute the mutated furin cleavage site.

An exemplary amino acid sequence of a
SARS-CoV-2 B.1.617 spike protein con-
taining six stabilizing proline substi-
tutions (shown in bold underline) is:
(SEQ ID NO: 6)
MFVFLVLLPLVSSQCVNLTTRTQLPPAYTNSFTRGVYYPDKVFRSS
VLHSTQDLFLPFFSNVTWFHAIHVSGTNGTKRFDNPVLPFNDGVYF
ASTEKSNIIRGWIFGTTLDSKTQSLLIVNNATNVVIKVCEFQFCND
PFLDVYYHKNNKSWMKSEFRVYSSANNCTFEYVSQPFLMDLEGKQG
NFKNLREFVFKNIDGYFKIYSKHTPINLVRDLPQGFSALEPLVDLP
IGINITRFQTLLALHRSYLTPGDSSSGWTAGAAAYYVGYLQPRTFL
LKYNENGTITDAVDCALDPLSETKCTLKSFTVEKGIYQTSNFRVQP
TESIVRFPNITNLCPFGEVFNATRFASVYAWNRKRISNCVADYSVL
YNSASFSTFKCYGVSPTKLNDLCFTNVYADSFVIRGDEVRQIAPGQ
TGKIADYNYKLPDDFTGCVIAWNSNNLDSKVGGNYNYRYRLFRKSN
LKPFERDISTEIYQAGSTPCNGVQGFNCYFPLQSYGFQPTNGVGYQ
PYRVVVLSFELLHAPATVCGPKKSTNLVKNKCVNFNFNGLTGTGVL
TESNKKFLPFQQFGRDIADTTDAVRDPQTLEILDITPCSFGGVSVI
TPGTNTSNQVAVLYQGVNCTEVPVAIHADQLTPTWRVYSTGSNVFQ
TRAGCLIGAEHVNNSYECDIPIGAGICASYQTQTNSRGSASSVASQ
SIIAYTMSLGAENSVAYSNNSIAIPTNFTISVTTEILPVSMTKTSV
DCTMYICGDSTECSNLLLQYGSFCTQLNRALTGIAVEQDKNTQEVF
AQVKQIYKTPPIKDFGGFNFSQILPDPSKPSKRSPIEDLLFNKVTL
ADAGFIKQYGDCLGDIAARDLICAQKFNGLTVLPPLLTDEMIAQYT
SALLAGTITSGWTFGAGPALQIPFPMQMAYRFNGIGVTQNVLYENQ
KLIANQFNSAIGKIQDSLSSTPSALGKLQDVVNQNAQALNTLVKQL
SSNFGAISSVLNDILSRLDPPEAEVQIDRLITGRLQSLQTYVTQQL
IRAAEIRASANLAATKMSECVLGQSKRVDFCGKGYHLMSFPQSAPH
GVVFLHVTYVPAHEKNFTTAPAICHDGKAHFPREGVFVSNGTHWFV
TQRNFYEPQIITTDNTFVSGNCDVVIGIVNNTVYDPLQPELDSFKE
ELDKYFKNHTSPDVDLGDISGINASVVNIQKEIDRLNEVAKNLNES
LIDLQELGKYEQYIKWPWYIWLGFIAGLIAIVMVTIMLCCMTSCCS
CLKGCCSCGSCCKFDEDDSEPVLKGVKLHYT,

wherein residues 13-685 constitute the S1 subunit, residues 686-1273 constitute the S2 subunit, and residues 682-685 (bolded) constitute the mutated furin cleavage site.

An exemplary amino acid sequence of a
SARS-CoV-2 B.1.617.2 spike protein con-
taining six stabilizing proline substi
tutions (shown in bold underline) is:
(SEQ ID NO: 7)
MFVFLVLLPLVSSQCVNLRTRTQLPPAYTNSFTRGVYYPDKVFRSS
VLHSTQDLFLPFFSNVTWFHAIHVSGTNGTKRFDNPVLPFNDGVYF
ASTEKSNIIRGWIFGTTLDSKTQSLLIVNNATNVVIKVCEFQFCND
PFLDVYYHKNNKSWMESGVYSSANNCTFEYVSQPFLMDLEGKQGNF
KNLREFVFKNIDGYFKIYSKHTPINLVRDLPQGFSALEPLVDLPIG
INITRFQTLLALHRSYLTPGDSSSGWTAGAAAYYVGYLQPRTFLLK
YNENGTITDAVDCALDPLSETKCTLKSFTVEKGIYQTSNFRVQPTE
SIVRFPNITNLCPFGEVFNATRFASVYAWNRKRISNCVADYSVLYN
SASFSTFKCYGVSPTKLNDLCFTNVYADSFVIRGDEVRQIAPGQTG
KIADYNYKLPDDFTGCVIAWNSNNLDSKVGGNYNYRYRLFRKSNLK
PFERDISTEIYQAGSKPCNGVEGFNCYFPLQSYGFQPTNGVGYQPY
RVVVLSFELLHAPATVCGPKKSTNLVKNKCVNFNFNGLTGTGVLTE
SNKKFLPFQQFGRDIADTTDAVRDPQTLEILDITPCSFGGVSVITP
GTNTSNQVAVLYQGVNCTEVPVAIHADQLTPTWRVYSTGSNVFQTR
AGCLIGAEHVNNSYECDIPIGAGICASYQTQTNSRGSASSVASQSI
IAYTMSLGAENSVAYSNNSIAIPTNFTISVTTEILPVSMTKTSVDC
TMYICGDSTECSNLLLQYGSFCTQLNRALTGIAVEQDKNTQEVFAQ
VKQIYKTPPIKDFGGFNFSQILPDPSKPSKRSPIEDLLFNKVTLAD
AGFIKQYGDCLGDIAARDLICAQKFNGLTVLPPLLTDEMIAQYTSA
LLAGTITSGWTFGAGPALQIPFPMQMAYRFNGIGVTQNVLYENQKL
IANQFNSAIGKIQDSLSSTPSALGKLQNVVNQNAQALNTLVKQLSS
NFGAISSVLNDILSRLDPPEAEVQIDRLITGRLQSLQTYVTQQLIR
AAEIRASANLAATKMSECVLGQSKRVDFCGKGYHLMSFPQSAPHGV
VFLHVTYVPAQEKNFTTAPAICHDGKAHFPREGVFVSNGTHWFVTQ
RNFYEPQIITTDNTFVSGNCDVVIGIVNNTVYDPLQPELDSFKEEL
DKYFKNHTSPDVDLGDISGINASVVNIQKEIDRLNEVAKNLNESLI
DLQELGKYEQYIKWPWYIWLGFIAGLIAIVMVTIMLCCMTSCCSCL
KGCCSCGSCCKFDEDDSEPVLKGVKLHYT,

wherein residues 13-685 constitute the S1 subunit, residues 686-1271 constitute the S2 subunit, and residues 680-683 (bolded) constitute the mutated furin cleavage site.

An exemplary amino acid sequence of a
SARS-CoV-2 B.1.1.7 spike protein con-
taining six stabilizing proline substi-
tutions is:
(SEQ ID NO: 8)
MFVFLVLLPLVSSQCVNLTTRTQLPPAYTNSFTRGVYYPDKVFRSS
VLHSTQDLFLPFFSNVTWFHAISGTNGTKRFDNPVLPFNDGVYFAS
TEKSNIIRGWIFGTTLDSKTQSLLIVNNATNVVIKVCEFQFCNDPF
LGVYHKNNKSWMESEFRVYSSANNCTFEYVSQPFLMDLEGKQGNFK
NLREFVFKNIDGYFKIYSKHTPINLVRDLPQGFSALEPLVDLPIGI
NITRFQTLLALHRSYLTPGDSSSGWTAGAAAYYVGYLQPRTFLLKY
NENGTITDAVDCALDPLSETKCTLKSFTVEKGIYQTSNFRVQPTES
IVRFPNITNLCPFGEVFNATRFASVYAWNRKRISNCVADYSVLYNS
ASFSTFKCYGVSPTKLNDLCFTNVYADSFVIRGDEVRQIAPGQTGK
IADYNYKLPDDFTGCVIAWNSNNLDSKVGGNYNYLYRLFRKSNLKP
FERDISTEIYQAGSTPCNGVEGFNCYFPLQSYGFQPTYGVGYQPYR
VVVLSFELLHAPATVCGPKKSTNLVKNKCVNFNFNGLTGTGVLTES
NKKFLPFQQFGRDIDDTTDAVRDPQTLEILDITPCSFGGVSVITPG
TNTSNQVAVLYQDVNCTEVPVAIHADQLTPTWRVYSTGSNVFQTRA
GCLIGAEHVNNSYECDIPIGAGICASYQTQTNSHRRARSVASQSII
AYTMSLGAENSVAYSNNSIAIPINFTISVTTEILPVSMTKTSVDCT
MYICGDSTECSNLLLQYGSFCTQLNRALTGIAVEQDKNTQEVFAQV
KQIYKTPPIKDFGGFNFSQILPDPSKPSKRSFIEDLLFNKVTLADA
GFIKQYGDCLGDIAARDLICAQKFNGLTVLPPLLTDEMIAQYTSAL
LAGTITSGWTFGAGAALQIPFAMQMAYRFNGIGVTQNVLYENQKLI
ANQFNSAIGKIQDSLSSTASALGKLQDVVNQNAQALNTLVKQLSSN
FGAISSVLNDILARLDKVEAEVQIDRLITGRLQSLQTYVTQQLIRA
AEIRASANLAATKMSECVLGQSKRVDFCGKGYHLMSFPQSAPHGVV
FLHVTYVPAQEKNFTTAPAICHDGKAHFPREGVFVSNGTHWFVTQR
NFYEPQIITTHNTFVSGNCDVVIGIVNNTVYDPLQPELDSFKEELD
KYFKNHTSPDVDLGDISGINASVVNIQKEIDRLNEVAKNLNESLID
LQELGKYEQYIKWPWYIWLGFIAGLIAIVMVTIMLCCMTSCCSCLK
GCCSCGSCCKFDEDDSEPVLKGVKLHYT.
An exemplary amino acid sequence of a
SARS-CoV spike protein containing six
stabilizing proline substitutions
(shown in bold underline) is:
(SEQ ID NO: 9)
MFIFLLFLTLTSGSDLDRCTTFDDVQAPNYTQHTSSMRGVYYPDEI
FRSDTLYLTQDLFLPFYSNVTGFHTINHTFDNPVIPFKDGIYFAAT
EKSNVVRGWVFGSTMNNKSQSVIIINNSTNVVIRACNFELCDNPFF
AVSKPMGTQTHTMIFDNAFNCTFEYISDAFSLDVSEKSGNFKHLRE
FVFKNKDGFLYVYKGYQPIDVVRDLPSGFNTLKPIFKLPLGINITN
FRAILTAFSPAQDTWGTSAAAYFVGYLKPTTFMLKYDENGTITDAV
DCSQNPLAELKCSVKSFEIDKGIYQTSNFRVVPSGDVVRFPNITNL
CPFGEVFNATKFPSVYAWERKKISNCVADYSVLYNSTFFSTFKCYG
VSATKLNDLCFSNVYADSFVVKGDDVRQIAPGQTGVIADYNYKLPD
DFMGCVLAWNTRNIDATSTGNYNYKYRYLRHGKLRPFERDISNVPF
SPDGKPCTPPALNCYWPLNDYGFYTTTGIGYQPYRVVVLSFELLNA
PATVCGPKLSTDLIKNQCVNFNFNGLTGTGVLTPSSKRFQPFQQFG
RDVSDFTDSVRDPKTSEILDISPCSFGGVSVITPGTNASSEVAVLY
QDVNCTDVSTAIHADQLTPAWRIYSTGNNVFQTQAGCLIGAEHVDT
SYECDIPIGAGICASYHTVSLLRSTSQKSIVAYTMSLGADSSIAYS
NNTIAIPTNFSISITTEVMPVSMAKTSVDCNMYICGDSTECANLLL
QYGSFCTQLNRALSGIAAEQDRNTREVFAQVKQMYKTPTLKYFGGF
NFSQILPDPLKPTKRSPIEDLLFNKVTLADAGFMKQYGECLGDINA
RDLICAQKFNGLTVLPPLLTDDMIAAYTAALVSGTATAGWTFGAGP
ALQIPFPMQMAYRFNGIGVTQNVLYENQKQIANQFNKAISQIQESL
TTTPTALGKLQDVVNQNAQALNTLVKQLSSNFGAISSVLNDILSRL
DPPEAEVQIDRLITGRLQSLQTYVTQQLIRAAEIRASANLAATKMS
ECVLGQSKRVDFCGKGYHLMSFPQAAPHGVVFLHVTYVPSQERNFT
TAPAICHEGKAYFPREGVFVFNGTSWFITQRNFFSPQIITTDNTFV
SGNCDVVIGIINNTVYDPLQPELDSFKEELDKYFKNHTSPDVDLGD
ISGINASVVNIQKEIDRLNEVAKNLNESLIDLQELGKYEQYIKWPW
YVWLGFIAGLIAIVMVTILLCCMTSCCSCLKGACSCGSCCKFDEDD
SEPVLKGVKLHYT,

wherein residues 14-667 constitute the S1 subunit and residues 668-1255 constitute the S2 subunit.

An exemplary amino acid sequence of a MERS-CoV spike protein containing six stabilizing proline substitutions (shown in bold underline) is: MIHSVFLLMFLLTPTESYVDVGPDSVKSACIEVDIQQTFFDKTWPRPIDVSKADGIIYPQGRTYSNIT ITYQGLFPYQGDHGDMYVYSAGHATGTTPQKLFVANYSQDVKQFANGEVVRIGAAANSTGTVIISPST SATIRKIYPAFMLGSSVGNFSDGKMGRFFNHTLVLLPDGCGTLLRAFYCILEPRSGNHCPAGNSYTSF ATYHTPATDCSDGNYNRNASLNSFKEYFNLRNCTEMYTYNITEDEILEWFGITQTAQGVHLESSRYVD LYGGNMFQFATLPVYDTIKYYSIIPHSIRSIQSDRKAWAAFYVYKLQPLTFLLDFSVDGYIRRAIDCG FNDLSQLHCSYESFDVESGVYSVSSFEAKPSGSVVEQAEGVECDESPLLSGTPPQVYNEKRLVFTNCN YNLTKLLSLESVNDFTCSQISPAAIASNCYSSLILDYFSYPLSMKSDLSVSSAGPISQFNYKQSFSNP TCLILATVPHNLTTITKPLKYSYINKCSRLLSDDRTEVPQLVNANQYSPCVSIVPSTVWEDGDYYRKQ LSPLEGGGWLVASGSTVAMTEQLQMGFGITVQYGTDTNSVCPKLEFANDTKIASQLGNCVEYSLYGVS GRGVFQNCTAVGVRQQRFVYDAYQNLVGYYSDDGNYYCLRACVSVPVSVIYDKETKTHATLFGSVACE HISSTMSQYSRSTRSMLKRRDSTYGPLQTPVGCVLGLVNSSLFVEDCKLPLGQSLCALPDTPSTLTPR SVRSVPGEMRLASIAFNHPIQVDQLNSSYFKLSIPTNFSFGVTQEYIQTTIQKVTVDCKQYVCNGFQK CEQLLREYGQFCSKINQALHGANLRQDDSVRNLFASVKSSQSSPIIPGFGGDENLILLEPVSISTGSR SARSPIEDLLEDKVTIADPGYMQGYDDCMQQGPASARDLICAQYVAGYKVLPPLMDVNMEAAYTSSLL GSIAGVGWTAGLSPFAAIPFPQSIFYRLNGVGITQQVLSENQKLIANKENQALGAMQTGFTTTPEAFQ KVQDAVNNNAQALSKLASELSNTFGAISASIGDIIQRLDPPEQDAQIDRLINGRLTTLNAFVAQQLVR SESAALSAQLAKDKVNECVKAQSKRSGFCGQGTHIVSFVVNAPNGLYFMHVGYYPSNHIEVVSAYGLC DAANPTNCIAPVNGYFIKTNNTRIVDEWSYTGSSFYAPEPITSLNTKYVAPQVTYQNISTNLPPPLLG NSTGIDFQDELDEFFKNVSTSIPNFGSLTQINTTLLDLTYEMLSLQQVVKALNESYIDLKELGNYTYY NKWPWYIWLGFIAGLVALALCVFFILCC (SEQ ID NO: 10), wherein residues 49-658 constitute the S1 subunit and residues 779-1353 constitute the S2 subunit.

Mutations in the spike protein that define a variant/strain from its corresponding native (wildtype) virus are known in the art. For example, the SARS-CoV-2 B.1.351 strain can include one or more mutations at residues selected from L18, D80, D215, R246, K417, E484, N501, D614 and A701 in the spike protein relative to a wildtype SARS-CoV-2 spike protein (e.g., SEQ ID NO:2). In certain non-limiting embodiments, the SARS-CoV-2 B.1.351 strain includes one or more mutations from L18F, D80A, D215G, R246I, K417N, E484K, N501Y, D614G and A701V in the spike protein relative to a wildtype SARS-CoV-2 spike protein (e.g., SEQ ID NO:2). In certain embodiments, the SARS-CoV-2 B.1.351 strain includes all nine foregoing mutations in the spike protein relative to a wildtype SARS-CoV-2 spike protein (e.g., SEQ ID NO:2). Thus, In certain non-limiting embodiments, the open reading frame encodes a spike protein or subunit or other fragment thereof that includes one or more mutations at residues L18, D80, D215, R246, K417, E484, N501, D614 and A701, such as but not limited to one or more mutations selected from L18F, D80A, D215G, R246I, K417N, E484K, N501Y, D614G and A701V relative to a wildtype SARS-CoV-2 spike protein (e.g., SEQ ID NO:2).

The SARS-CoV-2 B.1.617 strain can include one or more mutations at residues selected from G142, E154, L452, E484, D614, P681, and Q1071 in the spike protein relative to a wildtype SARS-CoV-2 spike protein (e.g., SEQ ID NO:2). In certain non-limiting embodiments, the SARS-CoV-2 B.1.617 strain includes one or more mutations selected from G142D, E154K, L452R, E484Q, D614G, P681R, and Q1071H in the spike protein relative to a wildtype SARS-CoV-2 spike protein (e.g., SEQ ID NO:2). In certain embodiments, the SARS-CoV-2 B.1.617 strain includes all seven foregoing mutations in the spike protein relative to a wildtype SARS-CoV-2 spike protein (e.g., SEQ ID NO:2). Thus, In certain non-limiting embodiments, the open reading frame encodes a spike protein or subunit or other fragment thereof that includes one or more mutations at residues G142, E154, L452, E484, D614, P681, and Q1071, such as but not limited to one or more mutations selected from G142D, E154K, L452R, E484Q, D614G, P681R, and Q1071H relative to a wildtype SARS-CoV-2 spike protein (e.g., SEQ ID NO:2).

The encoded spike proteins or subunits or other fragments thereof can also have one or more mutations that stabilize the spike protein in a prefusion conformation. A spike protein “stabilized in a prefusion conformation” can include one or more amino acid substitutions, deletions, or insertions compared to a native coronavirus spike sequence that provide for increased retention of the prefusion conformation compared to coronavirus spike formed from a corresponding native coronavirus spike protein sequence. The stabilization of the prefusion conformation can be, for example, energetic stabilization (e.g., reducing the energy of the prefusion conformation relative to the post-fusion open conformation) and/or kinetic stabilization (for example, reducing the rate of transition from the prefusion conformation to the post-fusion conformation). Additionally, stabilization of the spike in the prefusion conformation can include an increase in resistance to denaturation compared to a corresponding native coronavirus spike protein sequence. Methods of determining if a coronavirus spike protein is in the prefusion conformation include, but are not limited to, negative-stain electron microscopy and antibody binding assays using a prefusion-conformation-specific antibody.

In certain embodiments, the open reading frame encodes a spike protein or subunit or other fragment thereof that includes one or more mutations that stabilize the spike protein in a prefusion conformation. Typically, the one or more mutations include one or more proline substitutions. In certain embodiments, the one or more proline substitutions are in the spike S2 subunit.

In certain non-limiting embodiments when the virus is a SARS-CoV-2, suitable proline substitutions can be selected from F817P, A892P, A899P, A942P, K986P, V987P, and combinations thereof. In a particular form, the encoded spike protein or subunit or other fragment thereof contains two mutations selected from F817P, A892P, A899P, A942P, K986P, V987P. For example, in a particular form, the encoded spike protein or subunit or other fragment thereof contains the following two mutations: K986P and V987P. In a particular form, the encoded spike protein or subunit or other fragment thereof contains four mutations selected from F817P, A892P, A899P, A942P, K986P, V987P. In some certain embodiments, the encoded spike protein or subunit or other fragment thereof contains all six mutations selected from F817P, A892P, A899P, A942P, K986P, V987P. In certain non-limiting embodiments, the aforementioned mutations are indicated relative to a wildtype SARS-CoV-2 spike protein sequence, such as the sequence set forth in SEQ ID NO:2.

In certain non-limiting embodiments, when the virus is a MERS-CoV, suitable proline substitutions can be selected from A889P, S966P, A973P, N1016P, V1060P, L1061P and combinations thereof. In certain non-limiting embodiments, the encoded spike protein or subunit or other fragment thereof contains two mutations selected from A889P, S966P, A973P, N1016P, V1060P, and L1061P. For example, in a particular form, the encoded spike protein or subunit or other fragment thereof contains two such mutations: V1060P and L1061P. In certain non-limiting embodiments, the encoded spike protein or subunit or other fragment thereof contains four mutations selected from A889P, S966P, A973P, N1016P, V1060P, and L1061P. In some certain embodiments, the encoded spike protein or subunit or other fragment thereof contains all six mutations selected from A889P, S966P, A973P, N1016P, V1060P, and L1061P. In certain non-limiting embodiments, the aforementioned mutations are indicated relative to a wildtype MERS-CoV spike protein sequence, such as the sequence set forth in SEQ ID NO:3.

In certain non-limiting embodiments, when the virus is a SARS-CoV, suitable proline substitutions can be selected from F799P, A874P, A881P, S924P, K968P, V969P and combinations thereof. In certain non-limiting embodiments, the encoded spike protein or subunit or other fragment thereof contains two mutations selected from F799P, A874P, A881P, S924P, K968P, and V969P. In certain non-limiting embodiments, the encoded spike protein or subunit or other fragment thereof contains four mutations selected from F799P, A874P, A881P, S924P, K968P, and V969P. In some certain embodiments, the encoded spike protein or subunit or other fragment thereof contains all six mutations selected from F799P, A874P, A881P, S924P, K968P, and V969P. In certain non-limiting embodiments, the aforementioned mutations are indicated relative to a wildtype SARS-CoV spike protein sequence, such as the sequence set forth in SEQ ID NO:4.

Additionally, or alternatively, the encoded spike protein or subunit or other fragment thereof can include one or more mutations at a protease cleavage site, such as an S1/S2 and/or S2′ protease cleavage site. In certain non-limiting embodiments, cleavage site mutations can inhibit protease cleavage of the spike protein. The protease cleavage site can be a furin cleavage site. An exemplary furin cleavage site is RRAR (SEQ ID NO:15). In certain non-limiting embodiments, the furin cleavage site is deleted or replaced with a different sequence. For example, the furin cleavage site can be replaced with GSAS (SEQ ID NO: 11). Alternatively, other flexible linkers can be used, such as GSSS (SEQ ID NO:16) or GSGS (SEQ ID NO:17).

In certain non-limiting embodiments, the open reading encodes a spike protein or subunit or other fragment thereof linked to a trimerization domain to promote trimerization of the spike protein or subunit or other fragment, and to stabilize the membrane proximal aspect of the spike protein or subunit or other fragment in a trimeric configuration. In certain non-limiting embodiments, the spike protein or subunit or other fragment thereof is linked to the trimerization domain at the C-terminus. Non-limiting examples of multimerization domains that promote stable trimers of soluble recombinant proteins include: the GCN4 leucine zipper (Harbury et al. 1993 Science 262:1401-1407), the trimerization motif from the lung surfactant protein (Hoppe et al. 1994 FEBS Lett 344:191-195), collagen (McAlinden et al. 2003 J Biol Chem 278:42200-42207), and the phage T4 fibritin Foldon (Miroshnikov et al. 1998 Protein Eng 11:329-414), any of which can be linked to a disclosed spike protein or subunit or other fragment (e.g., by linkage to the C-terminus of S2) to promote trimerization of the spike protein or subunit or other fragment. In some examples, the C-terminus of the S2 subunit of the spike protein can be linked to a T4 fibritin Foldon domain. In certain non-limiting embodiments, the T4 fibritin Foldon domain includes the amino acid sequence GYIPEAPRDGQAYVRKDGEWVLLSTF (SEQ ID NO:12), which adopts a β-propeller conformation, and can fold and trimerize in an autonomous way (Tao et al. 1997 Structure 5:789-798). Optionally, the heterologous trimerization domain can be connected to the spike protein or subunit or other fragment thereof via a peptide linker, such as an amino acid linker. Non-limiting examples of peptide linkers that can be used include glycine, serine, and glycine-serine linkers.

In some certain embodiments, the mRNA encodes a chimeric (hybrid) protein or subunit or other fragment thereof which has sequences from different viral species or variants. The different viral species or variants can be selected from any coronavirus known in the art or disclosed herein, and include, for example, SARS-CoV, MERS-CoV, SARS-CoV-2, and variants of each of the foregoing such as SARS-CoV-2 B.1.1.7, SARS-CoV-2 B.1.351 (including B.1.351.2 and B.1.351.3), SARS-CoV-2 B.1.617 (including B.1.617.1, B.1.617.2, and SARS-CoV-2 B.1.617.3), P.1, P.1.1, P.1.2, B.1.427, B.1.429, B.1.525, B.1.526, P.2, B.1.621, and B.1.621.1.

For example, a chimeric protein can include two or more spike proteins or subunits or other fragments thereof (in frame with each other) from distinct coronavirus species or variants. In certain non-limiting embodiments, a disclosed chimeric protein contains a first spike protein or subunit or other fragment thereof (e.g., ECD, S1, S2, NTD, RBD) of a coronavirus selected from SARS-CoV, MERS-CoV, SARS-CoV-2 and variants thereof, and a second spike protein or subunit or other fragment thereof (e.g., ECD, S1, S2, NTD, RBD) of a coronavirus selected from SARS-CoV, MERS-CoV, SARS-CoV-2 and variants thereof. In certain non-limiting embodiments, the first spike protein or subunit or other fragment thereof is from a different viral species or variant from the second spike protein or subunit or other fragment thereof. In some certain embodiments, the chimeric protein does not contain a linker or other domain intervening between the two spike proteins or subunits or other fragments thereof from distinct coronavirus species or variants.

Non-limiting examples of chimeric proteins include, a chimeric protein having an S1 subunit from SARS-CoV-2 B.1.351 and an S2 subunit from SARS-CoV-2 B.1.617 (e.g., B.1.617.2 or B.1.617.3), a chimeric protein having an S1 subunit from SARS-CoV and an S2 subunit from SARS-CoV-2 (e.g., native/wildtype, B.1.1.7, B.1.351, B.1.617.2, B.1.617.3), a chimeric protein having an S1 subunit from MERS-CoV and an S2 subunit from SARS-CoV-2 (e.g., native/wildtype, B.1.1.7, B.1.351, B.1.617.2, B.1.617.3), and a chimeric protein having an S1 subunit from MERS-CoV and an S2 subunit from SARS-CoV. Also disclosed are variants of the foregoing in which the viral species or strains from which the S1 and S2 subunits are derived are reversed. It is to be understood that while the foregoing examples describe S1 and S2 subunits, In certain non-limiting embodiments, the S1 and/or S2 subunits can be substituted with other spike proteins or subunits or other fragment thereof (e.g., ECD, NTD, RBD). Thus, other examples of chimeric proteins include also include chimeric proteins of SARS-CoV-2 spike (e.g., native/wildtype, B.1.1.7, B.1.351, B.1.617.2, B.1.617.3, or other variants) in which the RBD (R319-F541) is replaced by MERS-CoV RBD (E367-Y606) or SARS-CoV RBD (R306-F527). In any of the foregoing, the S2 subunit can include one or more mutations, such as the proline substitutions described above, that stabilize the spike protein in a prefusion conformation.

Different peptide ligation approaches (e.g., SpyCatcher-SpyTag, SpyCatcher002-SpyTag002, SpyCatcher003-SpyTag003, SpyLigase-SpyTag, SpyLigase-KTag, SnoopCatcher-SnoopTag, SnoopLigase-SnoopTagJr, SnoopLigase-DogTag, SpyDock-SpyTag002) can be used to generate the disclosed chimeric proteins. In certain non-limiting embodiments, each spike protein or subunit or other fragment thereof contains a SPY tag/catcher sequence positioned at the N-terminus or C-terminus. The SpyCatcher-SpyTag system was developed as a method for protein ligation. It is based on a modified domain from a Streptococcus pyogenes surface protein (SpyCatcher), which recognizes a cognate 13-amino-acid peptide (SpyTag). Upon recognition, the two form a covalent isopeptide bond between the side chains of a lysine in SpyCatcher and an aspartate in SpyT (Hatlem D., et al., Int J Mol Sci., 20 (9): 2129 (2019)). An internal isopeptide bond forms spontaneously between the ε-amine of lysine and the side chain carboxyl of aspartic acid. The reaction is catalyzed by the spatially adjacent glutamate. The resulting isopeptide bond confers high stability. SpyCatcher contains the reactive lysine and catalytic glutamate, whereas SpyTag includes the reactive aspartate. The two components recognize each other with high affinity and the isopeptide can form between SpyCatcher and SpyTag to form a covalently bound complex. Under experimental conditions relevant to life science research (room temperature, dilute protein concentrations), the reaction rates allow the bonds to form at high efficiency within minutes (Hatlem D., et al., 2019). This technology has been used, among other applications, to create covalently stabilized multi-protein complexes, for modular vaccine production, and to label proteins (e.g., for microscopy). The SpyTag system is versatile as the tag is a short, unfolded peptide that can be genetically fused to exposed positions in target proteins; similarly, SpyCatcher can be fused to reporter proteins such as GFP, and to epitope or purification tags.

An exemplary amino acid sequence of SPY tag is
(SEQ ID NO: 13)
RGVPHIVMVDAYKRYK.
An exemplary amino acid sequence of SPY catcher is
(SEQ ID NO: 14)
VTTLSGLSGEQGPSGDMTTEEDSATHIKESKRDEDGRELAGATMELRDSS
GKTISTWISDGHVKDFYLYPGKYTFVETAAPDGYEVATPIEFTVNEDGQV
TVDGEATEGDAHT.

In certain non-limiting embodiments, one component of the chimeric protein (e.g., S1) contains a SPY tag (e.g., SEQ ID NO:13) positioned at its C-terminus and a second component (e.g., S2) contains a SPY catcher (e.g., SEQ ID NO:14) positioned at its N-terminus. In certain non-limiting embodiments, one component of the chimeric protein (e.g., S1) contains a SPY tag (e.g., SEQ ID NO:13) positioned at its N-terminus and a second component (e.g., S2) contains a SPY catcher (e.g., SEQ ID NO:14) positioned at its C-terminus. In certain non-limiting embodiments, the chimeric protein is formed by covalent attachment of the two components mediated by SPY tag and SPY catcher (see, e.g., FIGS. 9-10) or alternative ligation system. It will be appreciated that the components of the chimeric protein can be different spike proteins or subunits thereof including ECD, S1, S2, NTD, and/or RBD.

Also disclosed are variants of any of the encoded proteins or peptides described herein (e.g., a spike protein or subunit or other fragment thereof). For example, the mRNA can include an open reading frame that encodes a variant of any of the disclosed spike proteins or subunits or other fragments thereof. In certain non-limiting embodiments, suitable encoded polypeptides include variants of any one of SEQ ID NOs: 2-10 having, for example, at least 70%, 75%, 80%, 85%, 90%, 95%, 96%, 97%, 98%, 99%, or more sequence identity to any one of SEQ ID NOs: 2-10.

Suitable variants can include at least one point mutation or substitution (e.g., 1, 2, 3, 4, 5 or more mutations) at any amino acid residue relative to a reference (e.g., SEQ ID NOs: 1-14, such as but not limited to SEQ ID NOs: 2-10). Amino acid substitutions in certain non-limiting embodiments include conservative amino acid substitutions, although non-conservative substitutions can also be used. Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

### B. Other Polynucleotides

Also provided are isolated nucleic acid molecules or polynucleotides that encode the disclosed mRNAs. In certain non-limiting embodiments, the nucleic acid molecule/polynucleotide is or includes DNA. The polynucleotide can include one or more promoters and/or a polyadenylation signal operably linked to a sequence encoding the mRNA. In certain non-limiting embodiments, the polynucleotide is, or is contained within, a plasmid. In certain non-limiting embodiments, the polynucleotide is, or is contained within, a vector, such as an expression vector.

Expression vectors include all those known in the art, such as cosmids, plasmids (e.g., naked or contained in liposomes), phagemids, artificial chromosomes (e.g., BACs, YACs), and viral vectors (e.g., vectors derived from lentiviruses, retroviruses, adenoviruses, and adeno-associated viruses) that incorporate the polynucleotide.

In certain non-limiting embodiments, a polynucleotide (e.g., the portion thereof encoding a mRNA) is operably linked to a control element, e.g., a transcriptional control element, such as a promoter. The transcriptional control element may be functional in either a eukaryotic cell, e.g., a mammalian cell, or a prokaryotic cell (e.g., bacterial or archaeal cell). In certain non-limiting embodiments, a polynucleotide (e.g., the portion thereof encoding a mRNA thereof) is operably linked to multiple control elements that allow expression of the polynucleotide sequence encoding a mRNA in either prokaryotic or eukaryotic cells. Depending on the host/vector system utilized, any of a number of suitable transcription and translation control elements, including constitutive and inducible promoters, transcription enhancer elements, transcription terminators, etc. may be used in the expression vector (e.g., U6 promoter, HI promoter, CMV promoter, T7 promoter, SV40 promoter, bGH poly(A) signal, SV40 poly(A) signal, etc.).

Numerous vectors and expression systems are commercially available from commercial vendors including Addgene, Novagen (Madison, WI), Clontech (Palo Alto, CA), Stratagene (La Jolla, CA), and Invitrogen/Life Technologies (Carlsbad, CA). Suitable expression vectors include, but are not limited to, viral vectors such as viral vectors based on vaccinia virus, poliovirus, adenovirus, adeno-associated virus, SV40, herpes simplex virus, human immunodeficiency virus, retroviral vectors (e.g., Murine Leukemia Virus, spleen necrosis virus, and vectors derived from retroviruses such as Rous Sarcoma Virus, Harvey Sarcoma Virus, avian leukosis virus, lentivirus, human immunodeficiency virus, myeloproliferative sarcoma virus, and mammary tumor virus), and the like. The viral vector can be derived from a DNA virus (e.g., dsDNA or ssDNA virus) or an RNA virus (e.g., a ssRNA virus).

Numerous suitable expression vectors are known to those of skill in the art, and many are commercially available, including, pXTI, pSG5 (Stratagene), pSVK3, pBPV, pMSG, pCDNA 3.1, and pSVLSV40 (Pharmacia). However, any other vector may be used so long as it is compatible with the host cell.

Any cell may be used in accordance with the foregoing. In certain non-limiting embodiments, the cell is a prokaryotic cell (e.g., an archaeal or bacterial cell). In certain non-limiting embodiments, the cell is E. coli. In other forms, the cell is a eukaryotic cell. For example, the cell can be a cell of a single-cell eukaryotic organism, a plant cell, an algal cell, a fungal cell (e.g., a yeast cell). The cell can be a mammalian cell. The mammalian cell can be human or non-human mammal, e.g., primate, bovine, ovine, porcine, canine, rodent, monkey, rat, or mouse cell.

Generation of the polynucleotides can be accomplished using any suitable genetic engineering techniques well known in the art, including, without limitation, the standard techniques of restriction endonuclease digestion, ligation, transformation, plasmid purification, and DNA sequencing, for example as described in Sambrook et al. (Molecular Cloning: A Laboratory Manual. Cold Spring Harbor Laboratory Press, N.Y. (1989)).

C. Delivery vehicles

Also provided are vehicles for delivering or introducing the disclosed nucleic acids and compositions thereof to a cell. For example, vehicles for the introduction or production (e.g., transcription) of the disclosed mRNAs in a cell or tissue are described. Such vehicles include polynucleotides, such as plasmids and other vectors described above, which contain sequences encoding the mRNA. In certain non-limiting embodiments, viral vectors, virus-like particles, and/or lipid nanoparticles contain or encapsulate the disclosed mRNAs or polynucleotides encoding the disclosed mRNAs.

i. AAV

In certain non-limiting embodiments, the vector encoding a vaccine antigen (e.g., mRNA) is a viral vector. In certain non-limiting embodiments, the viral vector is an adeno-associated virus (AAV) vector.

AAV is a non-pathogenic, single-stranded DNA virus that has been actively employed over the years for delivering therapeutic genes in both in vitro and in vivo systems (Choi, et al., Curr. Gene Ther., 5:299-310, (2005)). AAV belongs to the parvovirus family and is dependent on co-infection with other viruses, mainly adenoviruses, in order to replicate. Each end of the single-stranded DNA genome contains an inverted terminal repeat (ITR), which is the only cis-acting element required for genome replication and packaging. The single-stranded AAV genome contains three genes, Rep (Replication), Cap (Capsid), and aap (Assembly). These three genes give rise to at least nine gene products through the use of three promoters, alternative translation start sites, and differential splicing. These coding sequences are flanked by the ITRs. The Rep gene encodes four proteins (Rep78, Rep68, Rep52, and Rep40), while Cap expression gives rise to the viral capsid proteins (VP; VP1/VP2/VP3), which form the outer capsid shell that protects the viral genome, as well as being actively involved in cell binding and internalization. It is estimated that the viral coat is comprised of 60 proteins arranged into an icosahedral structure with the capsid proteins in a molar ratio of 1:1:10 (VP1: VP2: VP3).

Recombinant AAV vectors having no Rep and/or Cap genes can be non-integrating. In the absence of Rep proteins, ITR-flanked transgenes encoded within rAAV can form circular concatemers that persist as episomes in the nucleus of transduced cells. Because recombinant episomal DNA does not integrate into host genomes, it will eventually be diluted over time as the cell undergoes repeated rounds of replication. This will eventually result in the loss of the transgene and transgene expression.

The sequences placed between the ITRs will typically include a promoter, gene of interest (e.g., encoding a disclosed mRNA), and a terminator. The promoter can be naturally-occurring or non-naturally occurring. In many cases, strong, constitutively active promoters are desired for high-level expression of the gene of interest. Examples of promoters, include, but are not limited to, viral promoters, plant promoters and mammalian promoters. Commonly used promoters include the CMV (cytomegalovirus) promoter/enhancer, EF1a (elongation factor 1a), SV40 (simian virus 40), chicken β-actin and CAG (CMV, chicken β-actin, rabbit β-globin) and variants thereof. All of these promoters provide constitutively active, high-level gene expression in most cell types. Some of these promoters are subject to silencing in certain cell types, therefore this consideration can be evaluated for each application.

Examples of terminators include, but are not limited to, polyadenylation signal sequences. Examples of polyadenylation signal sequences include, but are not limited to, Bovine growth hormone (BGH) poly(A), SV40 late poly(A), rabbit beta-globin (RBG) poly(A), thymidine kinase (TK) poly(A) sequences, and any variants thereof.

The viral vectors (e.g., AAV vector) can also have one or more restriction site(s) located near the promoter sequence to provide for the insertion of nucleic acid sequences encoding a mRNA/protein of interest.

The AAV vector used in the disclosed compositions and methods can be a naturally occurring serotype of AAV including, but not limited to, AAV1, AAV2, AAV3, AAV4, AAV5, AAV6, AAV7, AAV8, AAV9, AAV10, AAV11, AAV12, artificial variants such as AAV.rhlO, AAV.rh32/33, AAV.rh43, AAV.rh64R1, rAAV2-retro, AAV-DJ, AAV-PHP.B, AAV-PHP.S, AAV-PHP.eB, or other engineered versions of AAV. In a particular form, the AAV vector is AAV9. These serotypes differ in their tropism, or the types of cells they infect, making AAV a very useful system for in certain embodiments transducing specific cell types. Typically, AAV vectors have a packaging limit of ˜4.7 kb. The AAV itself may be immunogenic, which in some settings, can be used for its adjuvant effects.

ii. Virus-Like Particles

In certain non-limiting embodiments, a virus-like particle (VLP) includes a disclosed encoded spike protein or subunit or other fragment thereof. VLPs are small particles that contain certain proteins from the outer coat of a virus and can be constructed to present these proteins as antigens on their coat. Typically, VLPs lack the viral components that are required for virus replication and thus represent a highly attenuated, replication-incompetent form of a virus. Thus, VLPs can be regarded as non-replicating, viral shells, derived from any of several viruses. The VLP can display a polypeptide (e.g., a spike protein encoded by a disclosed mRNA) that is analogous to that expressed on infectious virus particles and can elicit an immune response to the corresponding virus when administered to a subject.

VLPs can be derived from various viruses such as e.g. the hepatitis B virus or other virus families including Parvoviridae (e.g. adeno-associated virus), Retroviridae (e.g. HIV), and Flaviviridae (e.g. Hepatitis C virus). For a general review see Sorensen M R and Thomsen A R, APMIS 115 (11): 1177-93 (2007) and Guillén et al., Procedia in Vaccinology 2 (2), 128-133 (2010).

VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system.

Virus like particles and methods of their production are known and familiar to the person of ordinary skill in the art, and viral proteins from several viruses are known to form VLPs, including human papillomavirus, HIV (Kang et al., Biol. Chem. 380:353-64 (1999)), Semliki-Forest virus (Notka et al., Biol. Chem. 380:341-52 (1999)), human polyomavirus (Goldmann et al., J. Virol. 73:4465-9 (1999)), rotavirus (Jiang et al., Vaccine 17:1005-13 (1999)), parvovirus (Casal, Biotechnology and Applied Biochemistry, Vol 29, Part 2, pp 141-150 (1999)), canine parvovirus (Hurtado et al., J. Virol. 70:5422-9 (1996)), hepatitis E virus (Li et al., J. Virol. 71:7207-13 (1997)), and Newcastle disease virus.

The presence of VLPs following recombinant expression of viral proteins can be detected using conventional techniques known in the art. For example, the formation of VLPs can be detected by any suitable technique including techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation. VLPs can be isolated density gradient centrifugation and identified by characteristic density banding. See, for example, Baker et al. (1991) Biophys. J. 60:1445-1456; and Hagensee et al. (1994) J. Virol. 68:4503-4505; Vincente, J Invertebr Pathol., 2011; Schneider-Ohrum and Ross, Curr. Top. Microbiol. Immunol., 354:53073, 2012).

iii. Lipid Nanoparticles (LNPs)

In certain non-limiting embodiments, a disclosed mRNA or other disclosed polynucleotide (e.g., plasmid or vector) is formulated or encapsulated in a lipid nanoparticle. Non-limiting examples of lipid nanoparticles and methods of making them are described, for example, in Semple et al. (2010) Nat. Biotechnol. 28:172-176; Jayarama et al. (2012), Angew. Chem. Int. Ed., 51:8529-8533; and Maier et al. (2013) Molecular Therapy 21, 1570-1578, the contents of each of which are incorporated herein by reference in their entirety. Suitable lipid nanoparticle formulations are known in the art, see e.g., U.S. Pat. Nos. 9,950,065; 10,576,146; 10,485,884; 10,933,127; 10,703,789, and 10,702,600; which are hereby incorporated by reference in their entirety.

A lipid nanoparticle formulation may be influenced by, but not limited to, the selection of the cationic lipid component, the degree of cationic lipid saturation, the nature of the PEGylation, ratio of all components and biophysical parameters such as size. In one example by Semple et al. (Nature Biotech. 2010 28:172-176), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid can more effectively deliver siRNA to various antigen presenting cells (Basha et al. Mol Ther. 2011 19:2186-2200).

In certain non-limiting embodiments, the lipid nanoparticle includes one or more cationic lipids (e.g., ionizable cationic lipid), one or more helper lipids, one or more sterols, one or more PEG-modified lipids, or a combination thereof. In certain embodiments, the lipid nanoparticle includes at least one cationic lipid (e.g., ionizable cationic lipid), at least one helper lipid, at least one sterol, and at least one PEG-modified lipid. In certain non-limiting embodiments, the cationic lipid is an ionizable cationic lipid, the helper lipid is a neutral lipid, and the sterol is cholesterol.

The ionizable cationic lipids, which are pH-sensitive, attract anionic nucleic acids to form the core of self-assembling nanoparticle to ensure high encapsulation. Ionizable lipids are protonated at low pH, which makes them positively charged, but they remain neutral at physiological pH. The pH-sensitivity of ionizable lipids is beneficial for mRNA delivery in vivo, because neutral lipids have less interactions with the anionic membranes of blood cells and, thus, improve the biocompatibility of lipid nanoparticles. This also eliminates a mechanism of toxicity seen with permanently cationic molecules. Trapped in endosomes, in which the pH is lower than in the extracellular environment, ionizable lipids are protonated and, therefore, become positively charged, which may promote membrane destabilization and facilitate endosomal escape of the nanoparticle and/or encapsulated mRNA or other nucleic acid.

In certain non-limiting embodiments, a lipid nanoparticle includes about 35 to 45% cationic lipid, about 40% to 50% cationic lipid, about 50% to 60% cationic lipid, or about 55% to 65% cationic lipid. In certain non-limiting embodiments, the cationic lipid is selected from 2,2-dilinoleyl-4-dimethylaminoethyl [1,3]-dioxolane (DLin-KC2-DMA), dilinoleyl-methyl-4-dimethylaminobutyrate (DLin-MC3-DMA), and di ((Z)-non-2-en-1-yl) 9-((4-(dimethylamino) butanoyl)oxy) heptadecanedioate (L319). Suitable ionizable cationic lipids also include, without limitation, PNI ionizable lipid, SM-102, ALC-0315, DLin-DMA, DLin-D-DMA, DLin-MC3-DMA, DLin-KC2-DMA, DODMA, 1,2-dimyristoyl-sn-glycero-3-ethylphosphocholine (DMEPC), 1,2-di-O-octadecenyl-3-trimethylammonium propane (DOTMA), 1,2-dioleoyl-3-trimethylammonium propane (DOTAP), amino alcohol lipids and combinations thereof. Combinations of any of the foregoing cationic lipids can be used in various ratios.

Exemplary helper lipids include, but are not limited to, 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3-phosphocholin (POPC) and 1,2-dioleoyl-sn-glycero-3-phosphocholine (DOPC). In certain non-limiting embodiments, the LNP includes from about 0.5% to about 15% on a molar basis of the helper lipid e.g., from about 3 to about 12%, from about 5 to about 10% or about 15%, about 10%, or about 7.5% on a molar basis. Examples of helper lipids include, but are not limited to, DSPC, POPC, DPPC, DOPE and SM.

The LNPs can include a sterol component. For example, a sterol component may be included to confer suitable physicochemical and biological behavior. Such a sterol component may be selected from cholesterol or its derivative e.g., ergosterol or cholesterolhemisuccinate, but it is In certain non-limiting embodiments cholesterol. Cholesterol is often used in lipidic formulations because it is generally recognized that the presence of cholesterol decreases their permeability and protects them from the destabilizing effect of plasma or serum proteins. In certain embodiments, the sterol is cholesterol. In certain non-limiting embodiments, the sterol is a cholesterol-PEG conjugate. Combinations of any of the foregoing sterols can be used in various ratios. In certain non-limiting embodiments, the LNP includes from about 5% to about 50% on a molar basis of the sterol (e.g., about 15 to about 45%, about 20 to about 40%, about 40%, about 38.5%, about 35%, or about 31% on a molar basis).

Exemplary PEG-modified lipids include, but are not limited to, R-3-[(ω-methoxy-poly(ethyleneglycol)2000)carbamoyl)]-1,2-dimyristyloxypropyl-3-amine (PEG-c-DOMG or PEG-DOMG), 1,2-dimyristoyl-racglycero-3-methoxypolyethylene glycol-2000 (PEG-DMG), PEG2000-DMG, 1,2-Distearoyl-sn-glycerol, methoxypolyethylene glycol (PEG-DSG), 1,2-Dipalmitoyl-sn-glycerol, methoxypolyethylene glycol (PEG-DPG), PEG-CDMA, mPEG-OH, mPEG-AA (mPEG-CM), mPEG-CH₂CH₂CH₂—NH₂, MPEG-DMG, mPEG-N,N-Ditetradecylacetamide (ALC-0159), mPEG-DSPE, and mPEG-DPPE, and combinations thereof (further discussed in Reyes et al., J. Controlled Release, 107, 276-287 (2005), which is hereby incorporated by reference in its entirety). Combinations of any of the foregoing PEG-modified lipids can be used in various ratios.

In certain non-limiting embodiments, the LNPs include about 0.5% to 20% on a molar basis of the PEG or PEG-modified lipid (e.g., about 0.5 to 10%, about 0.5 to 5%, about 0.5%, about 1.5%, about 3.5%, or about 5% on a molar basis). In certain non-limiting embodiments, a PEG-modified lipid includes a PEG molecule of an average molecular weight of 2,000 Da. In certain non-limiting embodiments, a PEG-modified lipid includes a PEG molecule of an average molecular weight of less than 2,000 Da, for example around 1,500 Da, around 1,000 Da, or around 500 Da. The ratio of PEG in the lipid nanoparticle formulations may be increased or decreased and/or the carbon chain length of the PEG lipid may be modified from (e.g., from C14 to C18) to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0%, or 3.0% to 6.0% of the lipid molar ratio of PEG-modified lipid as compared to the cationic lipid, helper lipid and sterol.

In certain non-limiting embodiments, the LNP formulation may contain PEG-DMG 2000 (1,2-dimyristoyl-sn-glycero-3-phophoethanolamine-N-[methoxy (polyethylene glycol)-2000). In certain non-limiting embodiments, the LNP formulation may contain PEG-DMG 2000, a cationic lipid known in the art and at least one other component. In certain non-limiting embodiments, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example, the LNP formulation may contain PEG-DMG 2000, DLin-DMA, DSPC and cholesterol in a molar ratio of 2:40:10:48 (see, e.g., Geall et al., PNAS, 109 (36): 14604-9 (2012); PMID: 22908294).

In certain non-limiting embodiments, the lipid nanoparticle contains a lipid mixture in ratios of about 20-70% cationic lipid, 5-45% helper lipid, 20-55% cholesterol, 0.5-15% PEG-modified lipid; such as but not limited to about 20-60% ionizable cationic lipid, about 5-25% helper lipid, about 25-55% sterol, and/or about 0.5-15% PEG-modified lipid. In some certain embodiments, the lipid nanoparticle has a molar ratio of about 20-60% cationic lipid, about 5-25% helper lipid, 25-55% sterol, and 0.5-15% PEG-modified lipid. In certain non-limiting embodiments, the lipid nanoparticle includes about 25-75% of a cationic lipid, 0.5-15% of a helper lipid, 5-50% of a sterol, and 0.5-20% of PEG-modified lipid on a molar basis. In certain non-limiting embodiments, the lipid nanoparticle includes about 35-65% of a cationic lipid, 3-12% of a helper lipid, 15-45% of a sterol, and 0.5-10% of a PEG-modified lipid on a molar basis.

In certain non-limiting embodiments, the lipid nanoparticle has a mean diameter of about 10-500 nm, about 20-400 nm, about 30-300 nm, or about 40-200 nm. In certain non-limiting embodiments, the lipid nanoparticle has a mean diameter of about 20-100 nm, 40-100 nm, 50-100 nm, 50-150 nm, about 50-200 nm, about 80-100 nm or about 80-200 nm.

In certain non-limiting embodiments, the ratio of lipid to RNA (e.g., mRNA) in a lipid nanoparticle may be 5:1 to 20:1, 10:1 to 25:1, 15:1 to 30:1 and/or at least 30:1. In some certain embodiments, the ratio of lipid to mRNA in the disclosed lipid nanoparticles is in the range of about 5:1 to 20:1, inclusive. In some certain embodiments, the ratio of lipid to mRNA is 6:1. In certain non-limiting embodiments, the lipid to mRNA ratio is a molar ratio. For example, in some certain embodiments, the N:P molar ratio of a lipid nanoparticle containing mRNA is 6:1. The N:P ratio refers to the ratio of positively-chargeable polymer amine (N=nitrogen) groups to negatively-charged nucleic acid phosphate (P) groups. N:P ratio is an important physicochemical property of polymer-based gene delivery vehicles. The N:P character of a polymer/nucleic acid complex can influence many other properties such as its net surface charge, size, and stability.

Lipid nanoparticle formulations may be altered by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

The lipids in the LNPs can improve nanoparticle properties, such as particle stability, delivery efficacy, tolerability and biodistribution. For example, 1,2-distearoyl-sn-glycero-3-phosphocholine (DSPC), a phosphatidylcholine with saturated tails, has a melting temperature of ˜54° C. and a cylindrical geometry that allows DSPC molecules to form a lamellar phase, which stabilizes the structure of lipid nanoparticles. DSPC has been used in the mRNA-1273 and BNT162b2 COVID-19 vaccines. DOPE is a phosphoethanolamine with two unsaturated tails, which has a melting temperature of ˜30° C. and a conical shape120. DOPE tends to adopt an inverted hexagonal H (II) phase, which destabilizes endosomal membranes and facilitates endosomal escape of lipid nanoparticles.

Cholesterol can enhance particle stability by modulating membrane integrity and rigidity. The molecular geometry of cholesterol derivatives can further affect delivery efficacy and biodistribution of lipid nanoparticles. For example, cholesterol analogues with C-24 alkyl phytosterols increase the in vivo delivery efficacy of LNP-mRNA formulations. Here, the length of the hydrophobic tails of the cholesterol analogues, the flexibility of sterol rings and the polarity of hydroxy groups impact delivery efficacy.

PEG-modified lipids can have multiple effects on the properties of lipid nanoparticles. The amount of PEG-modified lipids can affect particle size and zeta potential. PEG-lipids can further contribute to particle stability by decreasing particle aggregation, and certain PEG modifications prolong the blood circulation time of nanoparticles by reducing clearance mediated by the kidneys and the mononuclear phagocyte system

Once they reach target cells, lipid nanoparticles can be internalized by multiple mechanisms, including macropinocytosis and clathrin-mediated and caveolae-mediated endocytosis. The endocytic pathway depends on the properties of the nanoparticle and the cell type. Following cellular internalization, lipid nanoparticles are usually trapped in endosomal compartments. Thus, endosomal escape is crucial for effective mRNA or other nucleic acid delivery. It is believed that positively charged lipids may facilitate electrostatic interaction and fusion with negatively charged endosomal membranes, resulting in the leak of mRNA or other nucleic acid molecules into the cytoplasm. Endosomal escape can be increased by optimizing the pKa values of ionizable lipids. Furthermore, the properties of lipidic tails can affect endosomal escape of lipid nanoparticles. For example, some lipids with branched tails show enhanced endosomal escape compared with their counterparts with linear tails, owing to stronger protonation at endosomal pH. In addition, modulating the type (for example, DSPC and DOPE) and ratio of lipids may improve endosomal escape. See Hou X., et al., Nat Rev Mater., 1-17. (2021) doi: 10.1038/s41578-021-00358-0 for a discussion of the design of lipid nanoparticles for mRNA delivery and the physiological barriers and suitable administration routes for lipid nanoparticle-mRNA systems.

### D. Pharmaceutical Formulations

Also provided are pharmaceutical formulations including one or more of the more disclosed compositions (e.g., mRNA, other polynucleotide such as plasmids and vectors, optionally provided in a disclosed delivery vehicle (e.g., AAV, VLP, LNP) and one or more pharmaceutically acceptable carriers, diluents, and/or excipients.

In certain non-limiting embodiments, a pharmaceutical composition or formulation includes a disclosed lipid nanoparticle with one or more disclosed mRNAs encapsulated in the LNP, and a pharmaceutically acceptable carrier, diluent, or excipient. In certain non-limiting embodiments, a pharmaceutical composition or formulation includes a disclosed lipid nanoparticle encapsulating one or more disclosed polynucleotides (e.g., plasmids or vectors) encapsulated in the LNP, and a pharmaceutically acceptable carrier, diluent, or excipient. In certain non-limiting embodiments, a pharmaceutical composition or formulation includes a AAV vector containing a sequence encoding a disclosed mRNA and a pharmaceutically acceptable carrier, diluent, or excipient. In certain non-limiting embodiments, a pharmaceutical composition or formulation includes a VLP containing one or more encoded polypeptides (e.g., spike proteins or subunits or other fragment thereof) and a pharmaceutically acceptable carrier, diluent, or excipient.

Pharmaceutical compositions may optionally further include one or more additional active agents, e.g., therapeutic and/or prophylactic agents. General considerations in the formulation and/or manufacture of pharmaceutical agents may be found, for example, in Remington: The Science and Practice of Pharmacy 21st ed., Lippincott Williams & Wilkins, 2005.

The pharmaceutical compositions are In certain non-limiting embodiments sterile and contain an effective amount of the active compounds (e.g., mRNAs optionally encapsulated in LNPs, and optionally further agents) to generate the desired reaction or the desired effect. Pharmaceutical compositions are usually provided in a uniform dosage form and may be prepared in an appropriate manner. The pharmaceutical composition may for example be in the form of a solution or suspension. The pharmaceutical composition may include salts, buffer substances, preservatives, carriers, diluents and/or excipients all of which are In certain non-limiting embodiments pharmaceutically acceptable. Pharmaceutically acceptable refers to the non-toxicity of a material which does not interact with the action of the active component of the pharmaceutical composition.

The term “excipient” when used herein is intended to indicate all substances which may be present in a pharmaceutical composition and which are not active ingredients such as, e.g., carriers, binders, lubricants, thickeners, surface active agents, preservatives, emulsifiers, buffers, flavoring agents, or colorants. Pharmaceutically acceptable excipients include any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof.

In certain non-limiting embodiments, an excipient is approved for use in humans and for veterinary use. In certain non-limiting embodiments, an excipient is approved by United States Food and Drug Administration. In certain non-limiting embodiments, an excipient is pharmaceutical grade. In certain non-limiting embodiments, an excipient meets the standards of the United States Pharmacopoeia (USP), the European Pharmacopoeia (EP), the British Pharmacopoeia, and/or the International Pharmacopoeia.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly (vinyl-pyrrolidone) (crospovidone), sodium carboxymethyl starch (sodium starch glycolate) and combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof. In certain non-limiting embodiments, suitable buffer substances include acetic acid in a salt, citric acid in a salt, boric acid in a salt, and phosphoric acid in a salt.

Exemplary lubricating agents include, but are not limited to, magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, etc., and combinations thereof.

The pharmaceutical compositions may be administered via any conventional route, such as by parenteral administration including by injection or infusion. Administration is In certain non-limiting embodiments parenterally, e.g., intravenously, intraarterially, subcutaneously, intradermally or intramuscularly. The term “parenteral administration” refers to the administration in a manner other than through the digestive tract, as by intravenous or intramuscular injection. Systemic administration is a route of administration that is either enteral, i.e., administration that involves absorption through the gastrointestinal tract, or parenteral. In certain non-limiting embodiments, the pharmaceutical compositions can be administered by a route selected from, for example, intramuscular, intradermal, subcutaneous, intravenous, intra-arterial, intra-articular, intraperitoneal, intranasal, sublingual, tonsillar, oropharyngeal, or other parenteral and mucosal routes. Actual methods for preparing administrable compositions will be known or apparent to those skilled in the art.

Compositions suitable for parenteral administration usually include a sterile aqueous or nonaqueous preparation of the active compound(s), which is In certain non-limiting embodiments isotonic to the blood of the recipient. Examples of compatible carriers and solvents are water, Ringer's solution, U.S.P., and isotonic sodium chloride solution. In addition, usually sterile, fixed oils are used as solution or suspension medium. For this purpose, any bland fixed oil can be employed including synthetic mono- or diglycerides. Fatty acids such as oleic acid can be used in the preparation of injectables.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Aqueous solutions of the pharmaceutical formulations may be packaged for use as is or lyophilized. Lyophilized preparations can be combined with a sterile solution prior to administration for either single or multiple dosing.

### Vaccines

The disclosed compositions, including pharmaceutical compositions can be suitable for use as vaccines. Thus, vaccines are provided herein. A vaccine is a biological preparation that improves or provides immunity to a particular disease or infectious agent. In certain non-limiting embodiments, a vaccine includes a disclosed pharmaceutical composition, optionally in combination with one or more adjuvants. In certain non-limiting embodiments, a vaccine includes a disclosed lipid nanoparticle encapsulating one or more mRNAs, optionally in combination with one or more adjuvants.

In certain embodiments, a vaccine includes a lipid nanoparticle encapsulating a mRNA which encodes a coronavirus spike protein or subunit or other fragment thereof (e.g., ECD, S1, S2, NTD and/or RBD) derived from SARS-CoV, MERS-CoV, SARS-CoV-2, or variants thereof. In some certain embodiments, the vaccine includes a lipid nanoparticle encapsulating a mRNA which encodes a coronavirus spike protein or subunit or other fragment thereof derived from a SARS-CoV-2 variant, such as B.1.1.7, B.1.351, B.1.351.2, B.1.351.3, B.1.617.1, B.1.617.2, B.1.617.3, P.1, P.1.1, P.1.2, B.1.427 B.1.429, B.1.525, B.1.526, P.2, B.1.621 and B.1.621.1.

In certain non-limiting embodiments, the vaccine can be multivalent, including mRNAs encoding proteins from multiple pathogens. For example, In certain non-limiting embodiments, a vaccine includes a lipid nanoparticle encapsulating multiple mRNAs which collectively encodes coronavirus spike proteins or subunits or other fragments thereof (e.g., ECD, S1, S2, NTD and/or RBD) derived from SARS-CoV, MERS-CoV, and/or SARS-CoV-2, including variants thereof. In some certain embodiments, the vaccine includes a lipid nanoparticle encapsulating three mRNAs collectively encoding spike proteins or subunits or other fragments thereof (e.g., ECD, S1, S2, NTD and/or RBD) derived from SARS-CoV, MERS-CoV, and SARS-CoV-2 including variants thereof. In such forms, the multivalent vaccine can induce immunity against SARS-CoV, MERS-CoV, and SARS-CoV-2 concurrently.

In certain non-limiting embodiments, a vaccine includes a lipid nanoparticle encapsulating multiple mRNAs which collectively encode coronavirus spike proteins or subunits or other fragments thereof (e.g., ECD, S1, S2, NTD and/or RBD) derived from SARS-CoV and MERS-CoV, including variants thereof. In certain non-limiting embodiments, a vaccine includes a lipid nanoparticle encapsulating multiple mRNAs which collectively encode coronavirus spike proteins or subunits or other fragments thereof (e.g., ECD, S1, S2, NTD and/or RBD) derived from SARS-CoV-2 and MERS-CoV, including variants thereof. In certain non-limiting embodiments, a vaccine includes a lipid nanoparticle encapsulating multiple mRNAs which collectively encode coronavirus spike proteins or subunits or other fragments thereof (e.g., ECD, S1, S2, NTD and/or RBD) derived from SARS-CoV and SARS-CoV-2, including variants thereof.

In certain non-limiting embodiments, the mRNAs encoding proteins from multiple pathogens are provided in equivalent amounts, e.g., 1:1 ratio, 1:1:1 ratio, etc.

The vaccines can also include one or more mRNAs encoding chimeric proteins derived by combining sequences from multiple pathogens (e.g., from different viral species or variants). The chimeric protein can be a chimeric coronavirus spike protein which has sequences from different coronavirus species or variants, such as SARS-CoV, MERS-CoV, SARS-CoV-2, and variants of each of the foregoing, such as SARS-CoV-2 B.1.1.7, SARS-CoV-2 B.1.351 (including B.1.351.2 and B.1.351.3), SARS-CoV-2 B.1.617 (including B.1.617.1, B.1.617.2, and SARS-CoV-2 B.1.617.3), P.1, P.1.1, P.1.2, B.1.427 B. 1.429, B.1.525, B.1.526, P.2, B.1.621 and B.1.621.1.

In some certain embodiments, a vaccine includes a lipid nanoparticle encapsulating mRNAs encoding a chimeric protein containing two or more spike proteins or subunits or other fragments thereof (in frame with each other) from distinct coronavirus species or variants. In some certain embodiments, a vaccine includes a lipid nanoparticle encapsulating mRNAs encoding a chimeric protein which contains a first spike protein or subunit or other fragment thereof (e.g., ECD, S1, S2, NTD, RBD) of a coronavirus selected from SARS-CoV, MERS-CoV, SARS-CoV-2 and variants thereof, and a second spike protein or subunit or other fragment thereof (e.g., ECD, S1, S2, NTD, RBD) of a coronavirus selected from SARS-CoV, MERS-CoV, SARS-CoV-2 and variants thereof, wherein the first spike protein or subunit or other fragment thereof is from a different viral species or variant from the second spike protein or subunit or other fragment thereof.

In some certain embodiments, a vaccine includes a lipid nanoparticle encapsulating mRNAs encoding i) a chimeric protein having an S1 subunit from SARS-CoV-2 B.1.351 and an S2 subunit from SARS-CoV-2 B.1.617 (e.g., B.1.617.2 or B.1.617.3), ii) a chimeric protein having an S1 subunit from SARS-CoV and an S2 subunit from SARS-CoV-2 (e.g., native/wildtype, B.1.1.7, B.1.351, B.1.617.2, B.1.617.3), iii) a chimeric protein having an S1 subunit from MERS-CoV and an S2 subunit from SARS-CoV-2 (e.g., native/wildtype, B.1.1.7, B.1.351, B.1.617.2, B.1.617.3), and a chimeric protein having an S1 subunit from MERS-CoV and an S2 subunit from SARS-CoV. In certain non-limiting embodiments, the mRNAs (encapsulated in the nanoparticle) contain sequences encoding SPY-Tag/SPY-Catcher sequences, such that upon translation of the mRNAs, the chimeric protein is formed by covalent ligation of two peptide components encoded by the mRNAs (see, e.g., FIGS. 9-10).

Besides the LNP and encapsulated nucleic acid, the vaccine can also contain one or more excipients selected from sodium chloride, monobasic potassium phosphate, potassium chloride, dibasic sodium phosphate dihydrate, tromethamine, tromethamine hydrochloride, acetic acid, sodium acetate, and sucrose. In some certain embodiments, the vaccine includes sodium chloride, monobasic potassium phosphate, potassium chloride, dibasic sodium phosphate dihydrate, and sucrose. In some certain embodiments, the vaccine includes tromethamine, tromethamine hydrochloride, acetic acid, sodium acetate, and sucrose.

The disclosed vaccines can further include, or may be administered in combination with, one or more adjuvants. Adjuvants describe compounds which prolong, enhance, accelerate, and/or exacerbate an immune response. Various mechanisms are possible in this respect, depending on the type of adjuvants used. In certain non-limiting embodiments, the vaccines include, or are administered in combination with, one or more adjuvants. In certain non-limiting embodiments, the vaccines do not include, or are not administered in combination with, one or more adjuvants.

Non-limiting examples of suitable adjuvants include cytokines, such as monokines, lymphokines, interleukins or chemokines (e.g., IL-1, IL-2, IL-3, IL-4, IL-5, IL-6, IL-7, IL-8, IL-9, IL-10, IL-12, INFα, INF-γ, GM-CSF, LT-α), lipopolysaccharides (LPS), CD40 ligands, GP96, dsRNA, CpG oligodeoxynucleotides, growth factors (e.g. hGH), aluminium hydroxide, Freund's adjuvant or oil such as Montanide®, In certain non-limiting embodiments Montanide® ISA51, lipid-A and derivatives or variants thereof, oil-emulsions, saponins, and Pam3Cys. These adjuvants have the advantage in that they help to stimulate the immune system in a non-specific way, thus enhancing the immune response to a pharmaceutical product.

### III. Methods of Making

Methods of making the disclosed mRNAs, other polynucleotides, and compositions and pharmaceutical formulations thereof are provided.

Polynucleotides may be prepared according to any available technique including, but not limited to chemical synthesis, enzymatic synthesis, which is generally termed in vitro transcription (IVT) or enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

The process of design and synthesis of the primary constructs of the disclosure generally includes the steps of gene construction, mRNA production (either with or without modifications) and purification. In the enzymatic synthesis method, a target polynucleotide sequence encoding the polypeptide of interest is first selected for incorporation into a vector which will be amplified to produce a cDNA template. Optionally, the target polynucleotide sequence and/or any flanking sequences may be codon optimized. The cDNA template is then used to produce mRNA through in vitro transcription (IVT).

mRNAs may be made using standard laboratory methods and materials. In certain non-limiting embodiments, mRNAs are produced by in vitro transcription of a linear or circularized DNA template (e.g., plasmid or other expression vector) containing sequences encoding the mRNAs. Plasmids or other expression vectors can be linearized by methods known in the art, such as restriction enzymes. The linearization reaction may be purified using methods including, for example Invitrogen's PURELINK™ PCR Micro Kit (Carlsbad, Calif.), and HPLC based purification methods such as, but not limited to, strong anion exchange HPLC, weak anion exchange HPLC, reverse phase HPLC (RP-HPLC), and hydrophobic interaction HPLC (HIC-HPLC) and Invitrogen's standard PURELINK™ PCR Kit (Carlsbad, Calif.).

The DNA template may be transcribed using an in vitro transcription (IVT) system. The system typically includes a transcription buffer, nucleotide triphosphates (NTPs), an RNase inhibitor and a polymerase. The NTPs may be manufactured in house, may be selected from a supplier, or may be synthesized as described herein. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate modified nucleic acids.

The DNA template may be removed using methods known in the art such as, but not limited to, treatment with Deoxyribonuclease I (DNase I). RNA clean-up may also include a purification method such as, but not limited to, AGENCOURT® CLEANSEQ® system from Beckman Coulter (Danvers, Mass.), and HPLC based purification methods such as, but not limited to, strong anion exchange HPLC, weak anion exchange HPLC, reverse phase HPLC (RP-HPLC), and hydrophobic interaction HPLC (HIC-HPLC).

The mRNA construct may undergo capping and/or tailing reactions. A capping reaction may be performed by methods known in the art to add a 5′ cap to the 5′ end of the primary construct. Methods for capping include, but are not limited to, using a Vaccinia Capping enzyme (New England Biolabs, Ipswich, Mass.), optionally with a 2′-O methyl-transferase. If a poly(A) tail is not encoded in the DNA template and thus absent from the mRNA transcript, a poly(A) tailing reaction may be performed by methods known in the art, such as, but not limited to, poly(A) Polymerase mediated tailing.

Subsequently, mRNA clean-up may be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified mRNA” refers to one that is separated from at least one contaminant. Thus, a purified polynucleotide (e.g., DNA or RNA) is present in a form or setting different from that in which it is found in nature, or a form or setting different from that which existed prior to subjecting it to a treatment or purification method.

The disclosed mRNAs can be formulated by mixing the mRNA with LNPs at a set ratio. Methods for synthesis of LNPs are known in the art. See for example, WO 2010/054401; Heyes et. al, J. Control Release, 107, 276-287 (2005); Semple et. al, Nature Biotechnology, 28, 172-176 (2010); Love et. al, PNAS, 107, 1864-1869 (2010) and Akinc et. al, Nature Biotechnology, 26, 561-569 (2008), all of which are hereby incorporated by reference in their entirety.

In certain non-limiting embodiments, solutions of the lipids/sterols forming the LNPs are combined in the appropriate molar ratio and diluted with ethanol to a final desired lipid concentration. Solutions of mRNA at a desired concentration in water can be diluted in sodium citrate buffer. Formulations of the lipid and mRNA can be prepared by combining the synthesized lipid solution with the mRNA solution at a desired total lipid to mRNA ratio. The formulations can be dialyzed one or more times against phosphate buffered saline (PBS) to remove the ethanol and to achieve buffer exchange. The resulting nanoparticle suspension can be filtered and stored as appropriate or used in accordance with the disclosed methods.

### IV. Methods of Antigen Design

Methods for the design of antigens suitable for use in the disclosed compositions (e.g., vaccines) are also provided. In certain non-limiting embodiments, antigens can be designed using one or more approaches described in working Example 4. For example, antigens can be designed using artificial intelligence and/or machine learning approaches (see Example 4). The designed antigens can be based on any pathogen, such as a virus, for example, a coronavirus including, but not limited to, SARS-CoV, MERS-CoV, and SARS-CoV-2.

In certain non-limiting embodiments, a method of vaccine design includes compiling a catalogue of genomic sequences from a plurality of organisms (e.g., viruses) and alignment of the sequences. The method may further involve Principal components analysis and/or training and evaluating machine learning base models (e.g., supervised learning). In certain non-limiting embodiments, accuracy scores obtained from machine learning models can be used to determine pathogenicity (COPA) scores (e.g., using a statistical hypothesis test-based meta-model).

The method can further include kernel regression smoothing to identify pathogenicity hotspots across the various genome sequences, optionally using COPA scores combined with local maxima identification.

In certain non-limiting embodiments the method can include B-cell and/or T-cell epitope analysis. For example, sequences for reference antigenic proteins can be used to predict B cell epitopes. In certain non-limiting embodiments, linear B-cell epitopes probability scores can be obtained as described in Jespersen et al., 2017. For T-cell epitope, prediction of peptides binding to MHC class I and/or class II molecules can be performed using TepiTool (see, Paul et al., 2016) from the Immune Epitope Database (IEDB) Analysis Resource.

### V. Methods of Use

Methods of using the disclosed mRNAs, other polynucleotides, compositions and pharmaceutical formulations thereof, and vaccines are also provided.

A. Protein expression

In certain non-limiting embodiments, the mRNAs and other polynucleotides can be used in methods to express and/or purify a desired protein or peptide, such as a coronavirus spike protein or subunit or other fragment thereof. For example, In certain non-limiting embodiments, a method of producing a recombinant coronavirus spike protein c (e.g., ECD, NTD, RBD, S1, and/or S2) involves introducing an appropriate disclosed mRNA or other disclosed polynucleotide (e.g., plasmid, expression vector) to a host cell under conditions sufficient for expression thereof, thereby producing the recombinant spike protein. In certain non-limiting embodiments, the recombinant spike protein is stabilized in a prefusion conformation due to the presence of one or more mutations. In certain non-limiting embodiments, the recombinant spike protein is a chimeric/hybrid spike protein or subunit or other fragment thereof. In certain non-limiting embodiments, the foregoing methods can further include purifying the spike protein or subunit or other fragment thereof from the cell.

### B. Vaccination

The disclosed pharmaceutical compositions and vaccines can be used in methods of inducing an immune response or vaccination. Typically, the immune response is against a coronavirus, including antigens thereof, such as a spike protein or subunit or other fragment thereof. In certain non-limiting embodiments, a method of inducing an immune response in a subject involves administering to the subject a disclosed vaccine in an effective amount to generate the immune response.

In certain non-limiting embodiments, the immune response is specific to a coronavirus such as MERS-CoV, SARS-CoV, or SARS-CoV-2, including variants thereof. In certain non-limiting embodiments, the immune response is specific to native/wildtype SARS-CoV-2 or a SARS-CoV-2 variant such as B.1.1.7, B.1.351, B.1.351.2, B.1.351.3, B.1.617.1, B.1.617.2, and/or B.1.617.3. In certain non-limiting embodiments, the immune response is specific to multiple coronaviruses. For example, administration of a single (e.g., multivalent) vaccine can induce an immune response specific to two or more coronaviruses selected from MERS-CoV, SARS-CoV, or SARS-CoV-2. In certain non-limiting embodiments, the immune response is specific the three coronaviruses: MERS-CoV, SARS-CoV, or SARS-CoV-2, including variants of each of the foregoing.

In certain non-limiting embodiments, the immune response is a T cell response. In certain non-limiting embodiments, the immune response is a B cell response. In certain non-limiting embodiments, the immune response involves both a T cell and B cell response. In certain non-limiting embodiments, the immune response involves a neutralizing antibody response specific to the coronavirus spike protein or subunit or other fragment thereof. In certain non-limiting embodiments, the immune response inhibits coronavirus infection in the subject. In certain non-limiting embodiments, the immune response inhibits replication of the coronavirus in the subject. The immune response can be a protective immune response, for example a response that inhibits subsequent infection with the virus (e.g., SARS-CoV-2). Elicitation of the immune response can also be used to treat or inhibit viral infection and illnesses associated with the virus, such as COVID-19.

Administration of a disclosed vaccine can be for prophylactic or therapeutic purpose. When provided prophylactically, the vaccine is provided in advance of any symptom, for example, in advance of infection. The prophylactic administration serves to prevent or ameliorate the course of any subsequent infection. When provided therapeutically, the vaccine is provided at or after the onset of a symptom of infection, for example, after development of a symptom of SARS-CoV-2 infection or after diagnosis with a SARS-CoV-2 infection. The vaccine can thus be provided prior to the anticipated exposure to the virus (e.g., SARS-CoV-2) so as to attenuate the anticipated severity, duration or extent of an infection and/or associated disease symptoms, after exposure or suspected exposure to the virus, or after the actual initiation of an infection.

In certain non-limiting embodiments, the subject being vaccinated has been exposed to, is infected with, or is at risk of infection by the coronavirus. In certain non-limiting embodiments, the subject is immunocompromised. In certain non-limiting embodiments, the subject is human.

i. Effective Amounts and Dosage Regimens

The pharmaceutical compositions, vaccines and other compositions described herein are administered in effective amounts. For example, the vaccine is provided to a subject in an amount effective to induce or enhance an immune response. The effective amount achieves a desired response or effect alone or together with further doses. In the case of treatment of a particular disease or of a particular condition, the desired response can be inhibition of the course of the disease. This can include slowing down the progress of the disease and, in particular, interrupting or reversing the progress of the disease. The desired response in a treatment of a disease or of a condition may also be delay of the onset or a prevention of the onset of said disease or said condition.

An effective amount of an agent or composition (e.g., vaccine) can depend on the disease indication, the severeness of the disease, the individual parameters of the subject (e.g., age, physiological condition, size and weight, fitness, extent of symptoms, susceptibility factors, and the like), the duration of treatment, the type of an accompanying therapy (if present), the specific route of administration, as well as the specific pharmacology of the composition for eliciting the desired activity or biological response in the subject, and similar factors. Accordingly, the doses administered of the vaccines may depend on various of such parameters. In certain non-limiting embodiments, the vaccine is administered in an effective amount to elicit a desired immune response, for example, a T cell and/or B cell response, and/or a neutralizing antibody response.

In certain non-limiting embodiments, a vaccine can be provided in unit dosage form for use to induce an immune response in a subject. A unit dosage form contains a suitable single preselected dosage for administration to a subject, or suitable marked or measured multiples of two or more preselected unit dosages, and/or a metering mechanism for administering the unit dose or multiples thereof.

Vaccination can involve one or more doses or administrations of the vaccines. In certain non-limiting embodiments, a single dose of a vaccine is administered. In certain non-limiting embodiments, two or more doses of a vaccine are administered. The two or more doses can be administered on different days, for example, about 14-28 (e.g., 14, 21, or 28) days apart. In certain non-limiting embodiments, the two or more doses can be administered 1, 2, 3, 4, 5, 6 or more months apart.

In certain non-limiting embodiments, each administration of the vaccine provides a dose of about 1 μg, 3 μg, 10 μg, 25 μg, 30 μg, or 100 μg. In certain non-limiting embodiments, the effective amount of the vaccine is a total dose (e.g., over multiples administrations) of about 1-500 μg, inclusive.

Dosage regimens can be adjusted to provide an optimum prophylactic or therapeutic response. A vaccine can be used in coordinate (or prime-boost) vaccination protocols or combinatorial formulations. In certain non-limiting embodiments, coordinate immunization protocols employ separate vaccines, each directed toward eliciting an anti-viral immune response, such as an immune response to SARS-CoV-2 and variants thereof. Separate vaccines that elicit an antiviral immune response can be combined in a polyvalent vaccine composition administered to a subject in a single immunization step, or they can be administered separately (in monovalent vaccine compositions) in a coordinate (or prime-boost) immunization protocol. There can be several boosts, and each boost can be a vaccine presenting a different immunogen (e.g., spike protein or subunit or other fragment thereof) from the same or different virus.

The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. In certain non-limiting embodiments, the boost can be administered about two, about three to eight, or about four weeks following the prime, or about several months after the prime. In certain non-limiting embodiments, the boost can be administered about 5, about 6, about 7, about 8, about 10, about 12, about 18, about 24, months after the prime, or more or less time after the prime. Periodic additional boosts can also be used at appropriate time points to enhance the subject's immune memory. The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., prevention of infection or improvement in disease state (e.g., reduction in viral load). If such monitoring indicates that vaccination is sub-optimal, the subject can be boosted with an additional vaccine d and/or the vaccination parameters can be modified in a fashion expected to potentiate the immune response.

ii. Routes of Administration

The vaccines and other pharmaceutical compositions may be administered by any suitable route. Administration can be local or systemic. Exemplary routes of administration include, but are not limited to, enteral, gastroenterol, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal (through the eye), intracavernous injection (into the base of the penis), intravaginal, intrauterine, transdermal (diffusion through the intact skin for systemic distribution), transmucosal (diffusion through a mucous membrane), insufflation (snorting), and sublingual.

In certain non-limiting embodiments, administration is via intradermal or intramuscular injection, or via oral, intranasal or intratracheal administration. For example, administration can be via drops or sprays. In certain embodiments, administration is via intramuscular injection.

### VI. Kits

The disclosed polynucleotides, reagents, compositions, and other materials can be packaged together in any suitable combination as a kit useful for performing, or aiding in the performance of, the methods. It is useful if the components in a given kit are designed and adapted for use together in the method.

For example, kits including vaccines or other compositions for administration to a subject, may include a pre-measured dosage of the composition in a sterile needle, ampule, tube, container, or other suitable vessel. The kits may include instructions for dosages and dosing regimens. In certain non-limiting embodiments, the vaccine compositions are lyophilized. The kit may further include agents (e.g., saline, a buffered solution) and instructions to form a formulation for administration. The instructions may specify suitable storage conditions for the kit and components thereof.

Also provided are kits for protein production. Such kits can include a disclosed polynucleotide (e.g., plasmid or other expression vector), viruses, virus-like particles, and/or instructions for use. The kit can further include reagents and instructions for transfection or transduction of recipient cells.

### EXAMPLES

### Example 1: Generation and Characterization of the Immune Responses Induced by Wildtype SARS-CoV-2 Spike mRNA-LNP Vaccine

### Materials and Methods

### Plasmid Construction

The DNA sequences of B.1.351 and B.1.617 SARS-CoV-2 spikes for the mRNA transcription and pseudovirus assay were synthesized as gBlocks (IDT) and cloned by Gibson Assembly (NEB) into pcDNA3.1 plasmids. To improve expression and retain prefusion conformation, six prolines (HexaPro variant, 6P) were introduced to the SARS-CoV-2 spike sequence in the mRNA transcription plasmids. The plasmids for the pseudotyped virus assay including pHIV_{NL}GagPol and pCCNanoLuc2AEGFP were gifts from Dr. Bieniasz′ lab⁵¹. The C-terminal 19 amino acids were deleted in the SARS-CoV-2 spike sequence for the pseudovirus assay.

### Cell Culture

HEK293T (ThermoFisher) and 293T-hACE2 (gifted from Dr Bieniasz′ lab) cell lines were cultured in complete growth medium, Dulbecco's modified Eagle's medium (DMEM; Thermo fisher) supplemented with 10% Fetal bovine serum (FBS, Hyclone), 1% penicillin-streptomycin (Gibco) (D10 media for short). Cells were typically passaged every 1-2 days at a split ratio of 1:2 or 1:4 when the confluency reached at 80%.

mRNA Production by In Vitro Transcription and Vaccine Formulation

A linearized DNA template containing the B.1.351 variant (6P) or B.1.617 variant (6P) open reading frame flanked by 5′ untranslated region (UTR) and 3′ UTR sequences and terminated by an encoded polyA tail was used as template for transcription. The above DNA templates were obtained from circular plasmids pVP22b (B.1351 variant (6P)) and pVP29b (B.1.617 variant (6P)). pVP22b and pVP29b plasmids were linearized with BbsI restriction enzyme digestion and cleaned up with gel purification. A sequence-optimized mRNA encoding B.1.351 variant (6P) or B.1.617 variant (6P) protein was synthesized in vitro using an Hiscribe™ T7 ARCA mRNA Kit (with tailing) (NEB), with 50% replacement of uridine by N1-methyl-pseudouridine.

The mRNA was synthesized and purified following the manufacturer's instructions and kept frozen at −80° C. until further use. The mRNA was encapsulated in a lipid nanoparticle (Genvoy-ILM™, Precision Nanosystems) using the NanoAssemblr® Ignite™ machine (Precision Nanosystems) following the guidance of manufacturers. In brief, Genvoy-ILM™, containing ionizable, structural, helper and polyethylene glycol lipids were mixed with mRNA in acetate buffer, pH 5.0, at a ratio of 6:1 (Genvoy-ILM™: mRNA). The mixture was neutralized with Tris-Cl pH 7.5, sucrose was added as a cryoprotectant. The final solution was sterile filtered and stored frozen at −80° C. until further use. The particle size of mRNA-LNP was determined by DLS machine (DynaPro NanoStar, Wyatt, WDPN-06). The encapsulation and mRNA concentration were measured using Quant-iT™ RiboGreen™ RNA Assay Kit (Thermofisher).

### Animals

M. musculus (mice), 6-8 weeks old females of C57BL/6Ncr, were purchased from Charles River and used for the immunogenicity studies. Animals were housed in individually ventilated cages in a dedicated vivarium with clean food, water, and bedding. Animals were housed with a maximum of 5 mice per cage, at regular ambient room temperature (65-75° F., or 18-23° C.), 40-60% humidity, and a 14 h: 10 h light cycle. All experiments utilized randomized littermate controls.

### Mice Immunization and Sample Collection

A standard two-dose schedule given 21 days apart was adopted¹. 1 μg or 10 μg LNP-mRNA were diluted in 1×PBS and inoculated into mice intramuscularly for prime and boost injections. Control mice received PBS. Two weeks post-prime (day 14) and two weeks post-boost (day 35), sera were collected from experimental mice and utilized for ELISA and pseudovirus neutralization assays. Forty days (day 40) after prime, mice were euthanized for endpoint data collection. Splenocytes were collected for T cell stimulation and cytokine analysis, and single cell profiling. Lymphocytes were separately collected from mouse blood, spleen and draining lymph nodes and used for Bulk BCR and TCR profiling.

### Flow Cytometry

Spleens from three mice in LNP mRNA vaccine groups and four mice in PBS group were collected five days post boost. Mononuclear single-cell suspensions from whole mouse spleens were generated using the above method. 0.5 million splenocytes were resuspended with 200 μl into RPMI 1640 supplemented with 10% FBS, 1% penicillin-streptomycin antibiotic, Glutamax and 2 mM 2-mercaptoethonal, anti-mouse CD28 antibody (Biolegend, Clone 37.51) and seeded into 96-well plate overnight. The splenocytes were incubated for 6 hr at 37° C. in vitro with BrefeldinA (Biolegend) under three conditions: no peptide, PMA/Ionomycin, and PepTivator® SARS-CoV-2 Prot_S Complete peptide pool (Miltenyi Biotec, 15 mers with 11 amino acid overlap) covering the entire SARS-CoV-2 S protein.

Peptide pools were used at a final concentration of 200 ng/ml. Following stimulation, cells were washed with PBS before surface staining with LIVE/DEAD Fixable Dead Cell Stain (Invitrogen, 1:1000) and a surface stain cocktail containing the following antibodies: CD3 PE/Cy7 (Biolegend, Clone 17A2, 1:200), CD8a BV421 (Biolegend, Clone QA17A07, 1:200), CD4 FITC (Biolegend, Clone GK1.5, 1:200) in MACS buffer (D-PBS with 2 mM EDTA and 0.5% BSA) on ice for 20 min, cells were washed with MACS buffer then fixed and permeabilized using the BD Cytofix/Cytoperm fixation/permeabilization solution kit according to the manufacturer's instructions. Cells were washed in perm/wash solution for 5 min, and stained by intracellular staining for 30 min at 4° C. using a cocktail of the following antibodies: IFN-γ PE (Biolegend, Clone W18272D,1:500), TNF Percp-Cy5.5 (Biolegend, Clone MP6-XT22, 1:500), IL2 BV510 (Biolegend, Clone JES6-5H4, 1:500), IL4 BV605 (Biolegend, Clone 11B11,1:500), IL5 APC (Biolegend, Clone TRFK5,1:500) in MACS buffer. Finally, cells were washed in MACS for twice and resuspended in MACS buffer before running on BD FACSAria II Cell Sorter (BD). Analysis was performed using FlowJo software.

### ELISA

384-well ELISA plates were coated with 3 μg/ml of antigens overnight at 4 degrees. The antigen panel used in the ELISA assay includes SARS-CoV-2 spike S1+S2 ECD and RBD of 2019-nCoV (SINO, ECD 40589-V08B1 and RBD 40592-V08B), Indian variant B.1.617 (SINO, ECD 40589-V08B12 and RBD 40592-V08H88), South African variant (SINO, ECD 40589-V08B07 and RBD 40592-V08H85) and spike RBD of wild-type, South African variant and Indian variant. Plates were washed with PBS plus 0.5% Tween 20 (PBST) three times using the 50TS microplate washer (Fisher Scientific, NC0611021) and blocked with 0.5% BSA in PBST at room temperature for one hour. Plasma was serially diluted twofold or fourfold starting at a 1:2000 dilution. Samples were added to the coated plates and incubated at room temperature for one hour, followed by washes with PBST five times. Anti-mouse secondary antibody was diluted to 1:2500 in blocking buffer and incubated at room temperature for one hour. Plates were washed five times and developed with tetramethylbenzidine substrate (Biolegend, 421101). The reaction was stopped with 1 M phosphoric acid, and OD at 450 nm was determined by multimode microplate reader (PerkinElmer EnVision 2105). The binding response (OD450) was plotted against the dilution factor in log 10 scale to display the dilution-dependent response. The area under curve of the dilution-dependent response (Log 10 AUC) was calculated to evaluate the potency of the serum antibody binding to spike antigens.

### SARS-CoV-2 Pseudovirus Reporter and Neutralization Assays

HIV-1 based SARS-CoV-2 WT, B.1.351 variant, and B.1.617 variant pseudotyped virions were generated using respective spike sequences, and used in neutralization assays. Plasmid expressing a C-terminally truncated SARS-CoV-2 S protein (pSARS-CoV-2Δ19) was from Dr Bieniasz′ lab. Plasmids expressing a C-terminally truncated SARS-CoV-2 B.1.351 variant S protein (B.1.351 variant-Δ19) and SARS-CoV-2 B.1.617 variant S protein (B.1.617 variant-Δ19) were generated as above. The three plasmid-based HIV-1 pseudotyped virus systems were utilized to generate (HIV-1/NanoLuc2AEGFP)-SARS-CoV-2 particles, (HIV-1/NanoLuc2AEGFP)-B.1.351 variant particles, and B.1.617 variant particles. The reporter vector, pCCNanoLuc2AEGFP, and HIV-1 structural/regulatory proteins (pHIVNLGagPol) expression plasmid were gifts from Dr Bieniasz's lab. Briefly, 293T cells were seeded in 150 mm plates, and transfected with 21 μg pHIVNLGagPol, 21 μg pCCNanoLuc2AEGFP, and 7.5 μg of a SARS-CoV-2 SΔ19 or B.1.351 variant-Δ19 or SARS-CoV-2 SA SΔ19 plasmid, utilizing 198 μl PEI. At 48 h after transfection, 20-ml supernatant was harvested and filtered through a 0.45-μm filter, and concentrated before being aliquoted and frozen at −80° C.

The pseudovirus neutralization assays were performed on 293T-hACE2 cells. One day before, 293T-hACE2 cells were plated in a 96 well plate at 0.01×10⁶ cells per well. The following day, 55 μL aliquots of serially diluted serum plasma, collected from PBS or LNP-mRNA vaccine immunized mice and starting from 1:100 (5-fold serial dilution using complete growth medium), were mixed with the same volume of SARS-CoV-2 WT, B.1.351 variant, and B.1.617 variant pseudovirus. The mixture was incubated for 1 hr in a 37° C. incubator supplied with 5% CO₂. Then 100 μL of the mixtures were added into 96-well plates with 293T-hACE2 cells. Plates were incubated at 37° C. supplied with 5% CO₂. 48 hr later, 293T-hACE2 cells were collected and the GFP+ cells were analyzed with Attune N×T Acoustic Focusing Cytometer (Thermo Fisher). The 50% inhibitory concentration (IC50) was calculated with a four-parameter logistic regression using GraphPad Prism (GraphPad Software Inc.).

### Negative-Stain TEM

5 μl of the sample was deposited on a glow-discharged formvar/carbon-coated copper grid (Electron Microscopy Sciences, catalog number FCF400-Cu-50), incubated for 1 min and blotted away. The grid was washed briefly with 2% (w/v) uranyl formate (Electron Microscopy Sciences, catalog number 22450) and stained for 1 min with the same uranyl formate buffer. Images were acquired using a JEOL JEM-1400 Plus microscope with an acceleration voltage of 80 kV and a bottom-mount 4k×3k charge-coupled device camera (Advanced Microscopy Technologies, AMT).

### Statistical Analysis

The statistical methods are described in figure legends. The statistical significance was labeled as follows: n.s., not significant; * p<0.05; ** p<0.01; *** p<0.001; **** p<0.0001. Prism (GraphPad Software) and RStudio were used for these analyses.

### Replication, Randomization, Blinding and Reagent Validations

Replicate experiments have been performed for all key data shown in this study. Biological or technical replicate samples were randomized where appropriate. In animal experiments, mice were randomized by littermates. Experiments were not blinded.

NGS data processing were blinded using metadata. Subsequent analyses were not blinded.

Commercial antibodies were validated by the vendors and revalidated in house as appropriate. Custom antibodies were validated by specific antibody-antigen interaction assays, such as ELISA. Isotype controls were used for antibody validations.

Cell lines were authenticated by original vendors and revalidated in lab as appropriate. All cell lines tested negative for mycoplasma.

### Results

Design, Generation and Physical Characterization of Variant-Specific SARS-CoV-2 Spike mRNA-LNPs.

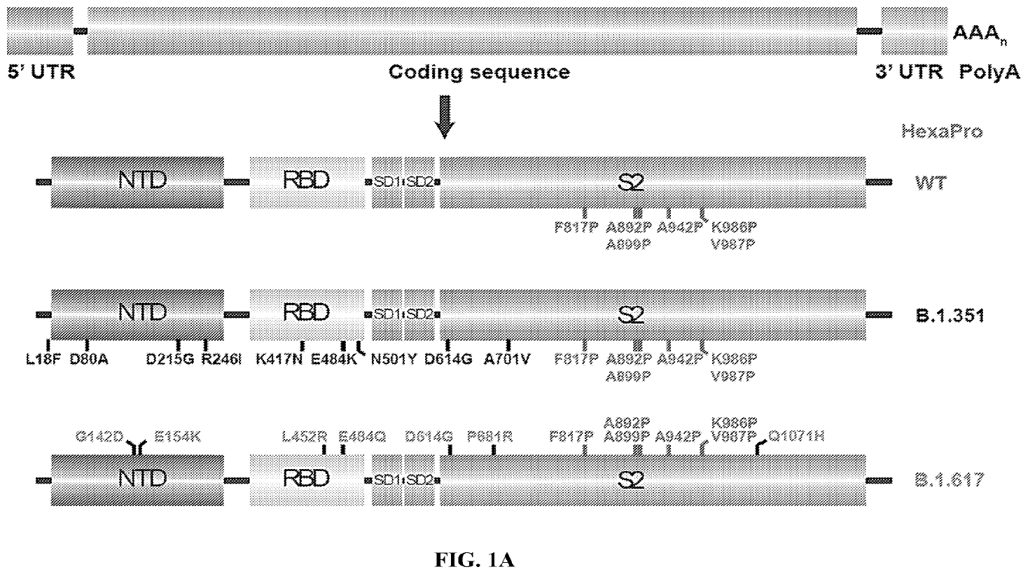

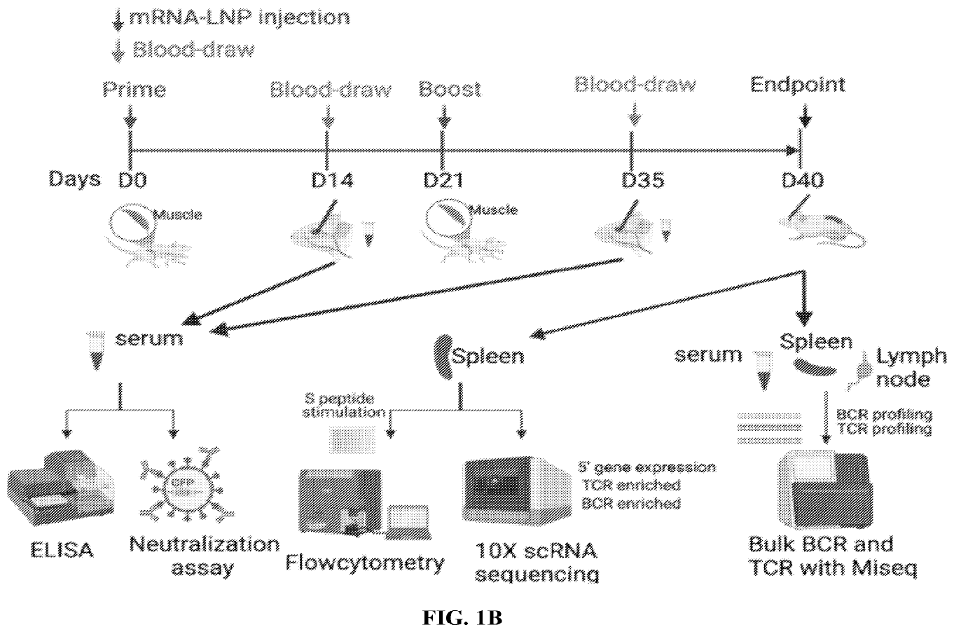

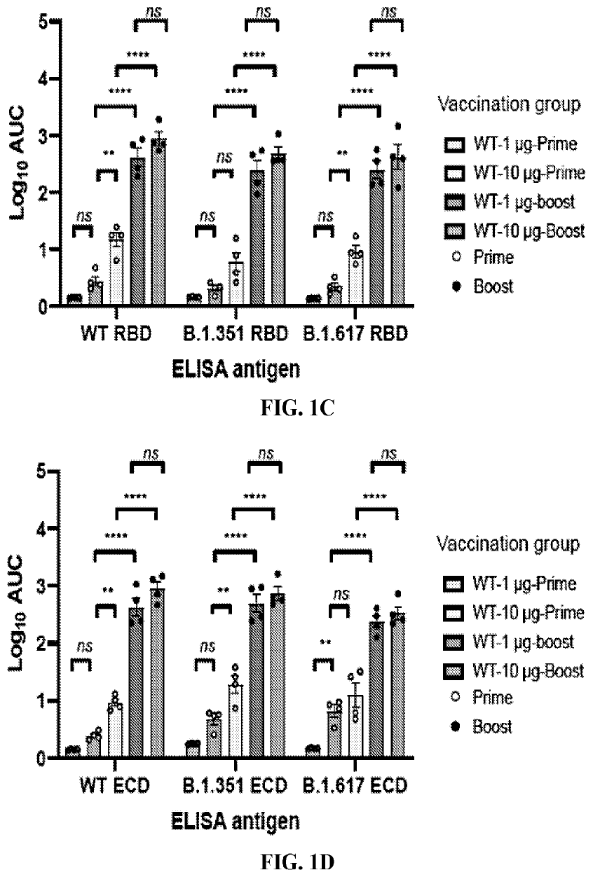

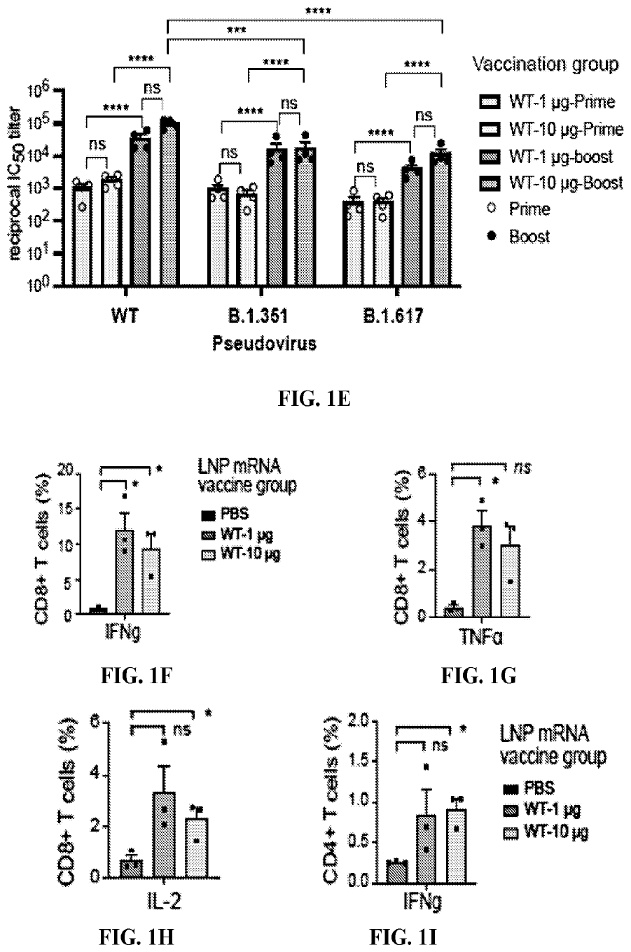

Nucleotide-modified mRNAs separately encoding full-length SARS-CoV-2 WT, B.1.351 and B.1.617 spike proteins were designed and generated. The HexaPro mutations (Wrapp, D. et al. Cryo-EM structure of the 2019-nCoV spike in the prefusion conformation. Science 367, 1260-1263, doi: 10.1126/science.abb2507 (2020)) were introduced and the furin cleavage site (Laczko, D. et al. A Single Immunization with Nucleoside-Modified mRNA Vaccines Elicits Strong Cellular and Humoral Immune Responses against SARS-CoV-2 in Mice. Immunity 53, 724-732 e727, doi: 10.1016/j.immuni.2020.07.019 (2020)) was replaced with a GSAS sequence to stabilize the prefusion state and preserve integrity of spike S1 and S2 subunits (FIG. 1A). The expression and receptor binding ability of modified spike proteins were confirmed by in vitro cell transfection followed by flow cytometry, where the spike binding to the human ACE2-Fc fusion protein was detected by PE-anti-Fc antibody. The spike mRNA was encapsulated in lipid nanoparticles (LNP), whose size and homogeneity were evaluated by dynamic light scattering (DLS) and transmission electron microscopy (TEM). The WT, B.1.351 and B.1.617 mRNA LNPs had mean diameters of 80.7±6.9 nm, 66.4±5.3 nm, and 72.2±5.8 nm with a monodispersed size distribution as determined by DLS and polydispersity indices of 0.08, 0.13 and 0.08, respectively.

The immunogenicity of the LNP-mRNA was assessed in C57BL/6Ncr mice by two intramuscular injections (doses) of 1 μg or 10 μg LNP-encapsulated mRNA, separated by 3 weeks (prime and boost, respectively) (FIG. 1B). Serum samples were collected two weeks after the prime and boost injections, and then subjected to ELISA and neutralization assays to evaluate the antibody response. These mice were sacrificed 40 days post vaccination, and the spleen, lymph nodes and blood cells were collected for downstream assays, including single cell transcriptomics sequencing (scRNA-seq), bulk and single cell BCR sequencing (BCR-seq) and TCR sequencing (TCR-seq), as well as flow cytometry (FIG. 1B). All procedures were standardized across all groups.

### Immune Responses Induced by WT-LNP-mRNA Vaccination in Mice

WT-LNP-mRNA induced dose-dependent binding antibody responses against spike ECD and RBD of SARS-CoV-2 WT, B.1.351 and B.1.617 variants after prime and boost injections (FIGS. 1C-1D). Compared to the post-prime immune response, orders of magnitude increase in immune response were observed after the boost injection, indicating that the second dose significantly boosted B cell immunity to SARS-CoV-2 antigen (FIGS. 1C-1D). Using a pseudovirus neutralization assay that has been widely reported to be consistent with authentic virus results (Chen, R. E. et al. In vivo monoclonal antibody efficacy against SARS-CoV-2 variant strains. Nature 596, 103-108, doi: 10.1038/s41586-021-03720-y (2021)), the serum samples from mice receiving the WT-LNP-mRNA vaccination also showed potent neutralization activity against all three variants, again with a strong prime-boost effect (FIG. 1E). However, the neutralization ability of WT-LNP-mRNA vaccinated sera was found to be several fold lower against either B.1.351 or B.1.617 as compared to the cognate WT pseudovirus (FIG. 1E). These observations are consistent with reports showing dramatic reduction in neutralization of B.1.351 and B.1.617 variants by vaccinated individuals' sera, convalescent sera, and therapeutic antibodies.

To evaluate the T cell response to the spike peptides, the splenocytes were isolated from mouse spleens 40 days post vaccination and the antigen-specific CD4⁺ and CD8⁺ T cell response to S peptide pools were determined by intracellular cytokine staining. WT-LNP-mRNA, at both low and high doses, induced reactive CD8⁺ T cells producing interferon γ (IFN-γ, IFNg), tumor necrosis factor α (TNF-α, TNFa), and interleukin 2 (IL-2) (FIG. 1F-1H), at levels consistent with previously reported studies (Laczko, D. et al. A Single Immunization with Nucleoside-Modified mRNA Vaccines Elicits Strong Cellular and Humoral Immune Responses against SARS-CoV-2 in Mice. Immunity 53, 724-732 e727, doi: 10.1016/j.immuni.2020.07.019 (2020); Corbett, K. S. et al. SARS-CoV-2 mRNA vaccine design enabled by prototype pathogen preparedness. Nature 586, 567-571, doi: 10.1038/s41586-020-2622-0 (2020)). WT-LNP-mRNA at both doses also induced reactive CD4⁺ T cells that produce IFN-γ⁺ (FIG. 1I), but little TNF-α, IL-2, IL-4, or IL-5. No difference in cytokine production was observed between vaccinated groups and the PBS group when cells were treated with vehicle (no peptide) or PMA/ionomycin. These results indicate that the WT-LNP-mRNA vaccine can induce potent spike protein specific CD4 and CD8 T cell responses.

### Example 2: Variant Specific SARS-CoV-2 Spike mRNA-LNP Vaccines Induce Potent Immune Responses

### Materials and Methods

In Vitro mRNA Expression

HEK293T cells were electroporated with mRNA encoding B.1351 variant (6P) or B.1.617 variant (6P) proteins using Neon™ Transfection System 10 μL Kit following the standard protocol provided by manufacturer. After 12 h, the cells were collected and resuspended in MACS buffer (D-PBS with 2 mM EDTA and 0.5% BSA). To detect surface-protein expression, the cells were stained with 10 μg/mL ACE2-Fc chimera (Genescript, Z03484) in MACS buffer for 30 min on ice. Thereafter, cells were washed twice in MACS buffer and incubated with PE-anti-human FC antibody (Biolegend, M1310G05) in MACS buffer for 30 min on ice. Live/Dead aqua fixable stain (Invitrogen) was used to assess viability. Data acquisition was performed on BD FACSAria II Cell Sorter (BD). Analysis was performed using FlowJo software.

### Results

### Binding and Neutralizing Antibody Responses of B.1.617-LNP-mRNA and B.1.351-LNP-mRNA

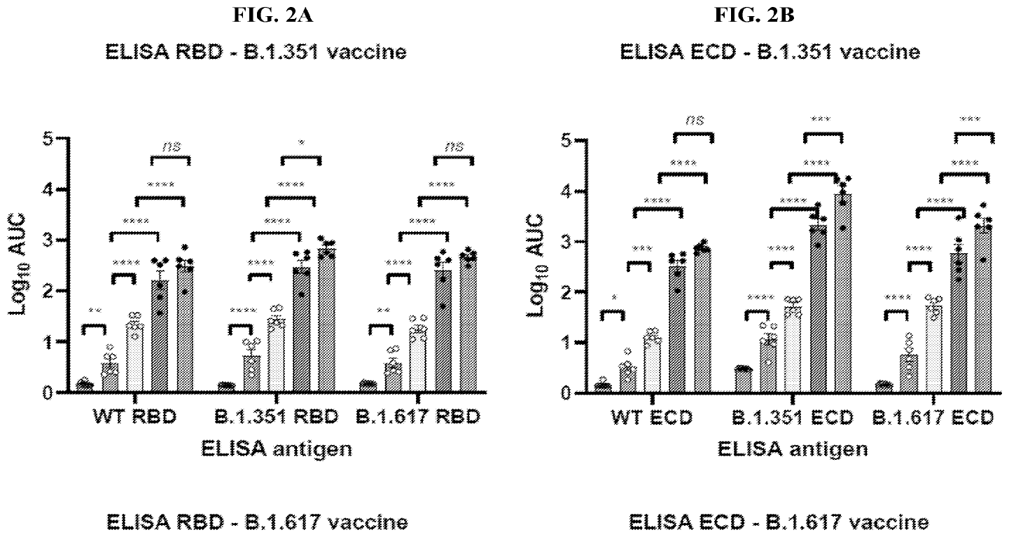

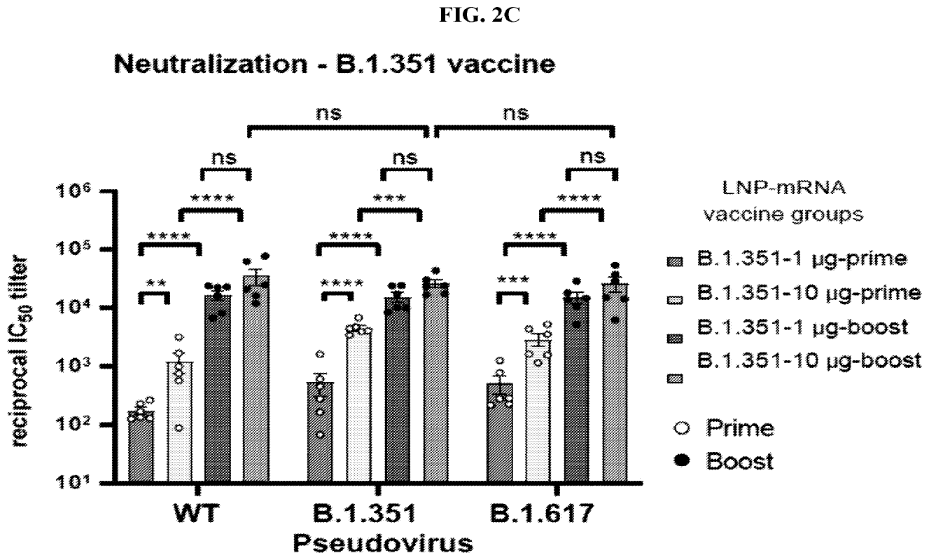

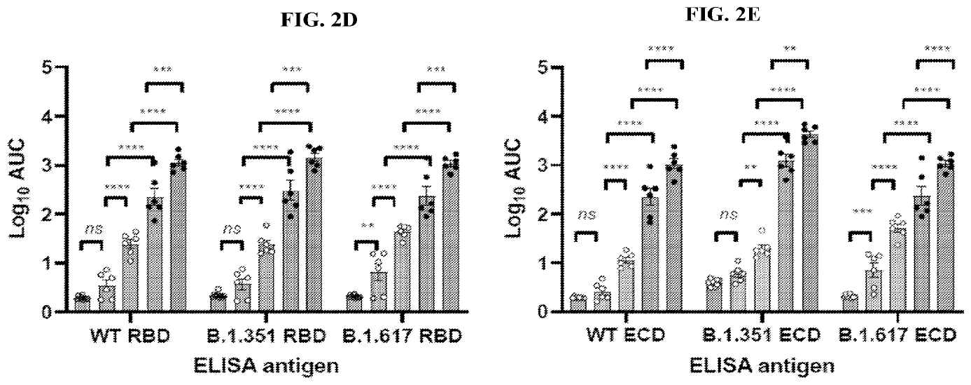

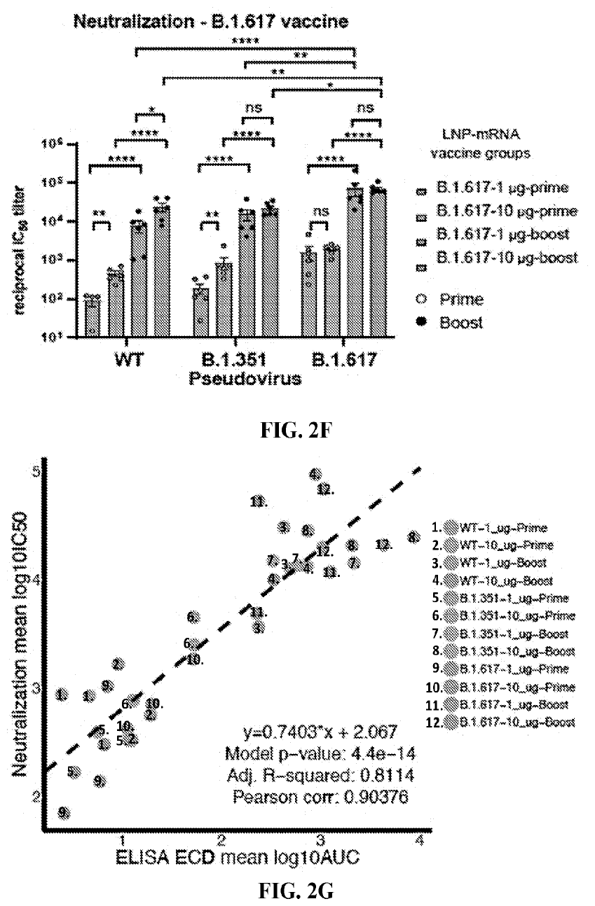

Both B.1.617-LNP-mRNA and B.1.351-LNP-mRNA induced dose-dependent binding antibody responses against spike ECD and RBD of SARS-CoV-2 WT, B.1.351 and B.1.617 variants (FIGS. 2A-2B, 2D-2E). The strong boost effect in ELISA was also observed for these two variant-specific LNP-mRNAs (FIGS. 2A-2B, 2D-2E). The dose-dependent effect was observed in both B.1.617-LNP-mRNA and B.1.351-LNP-mRNA groups across three types of ELISA antigens of both RBD and ECD, although the dose effect was less prominent in the post-boost samples, where both doses showed high titers at potential saturation level (FIGS. 2A-2B, 2D-2E). Relatively speaking, higher antibody responses was often observed with ECD antigen, suggesting an immunogenic domain other than RBD contributed to the additional response to spike ECD (FIGS. 2A-2B, 2D-2E). Overall, the binding intensity as measured by serum titer between RBD and ECD strongly correlated with each other across all vaccination groups.

The pseudovirus-neutralizing antibody response was then examined. Both B.1.617-LNP-mRNA and B.1.351-LNP-mRNA elicited potent neutralizing antibodies, a response which mirrored the trend of post-prime and post-boost responses observed by ELISA (FIGS. 2C, 2F). The initial level of neutralization was at 10²-10³ the level of reciprocal IC50 after priming for most groups (FIG. 2C, 2F). Consistent with findings by ELISA, an approximately two orders of magnitude increase in neutralization titer by boost was observed across all groups (for both vaccine candidates and for all three pseudovirus types) in the low dose (1 μg) setting, and there was an approximately one order of magnitude increase in the high dose (10 μg) setting (FIG. 2C, 2F). The dose effect of serum neutralization activity for both B.1.617-LNP-mRNA and B.1.351-LNP-mRNA was observed at priming for most groups, but negligible post boost (both 1 μg and 10 μg dose groups reached reciprocal IC50 titer of 104 level after boost) (FIG. 2C, 2F). Both B.1.617-LNP-mRNA and B.1.351-LNP-mRNA effectively neutralized all three SARS-CoV-2 pseudoviruses post boost at titers of 104 level (FIG. 2C, 2F). Interestingly, B.1.351-LNP-mRNA vaccinated animals neutralized all three SARS-CoV-2 pseudoviruses at similar levels post boost at both doses (FIG. 2C); while B.1.617-LNP-mRNA vaccinated animals showed significantly higher titer against its cognate B.1.617 pseudovirus (by several folds) (FIG. 2F). Overall, across all vaccination groups, the neutralization activity strongly correlated with ECD binding (FIG. 2G), which also holds true for RBD binding.

### B.1.617-LNP-mRNA and B.1.351-LNP-mRNA Elicited Strong Systemic T Cell Response Against SARS-CoV-2 Spike

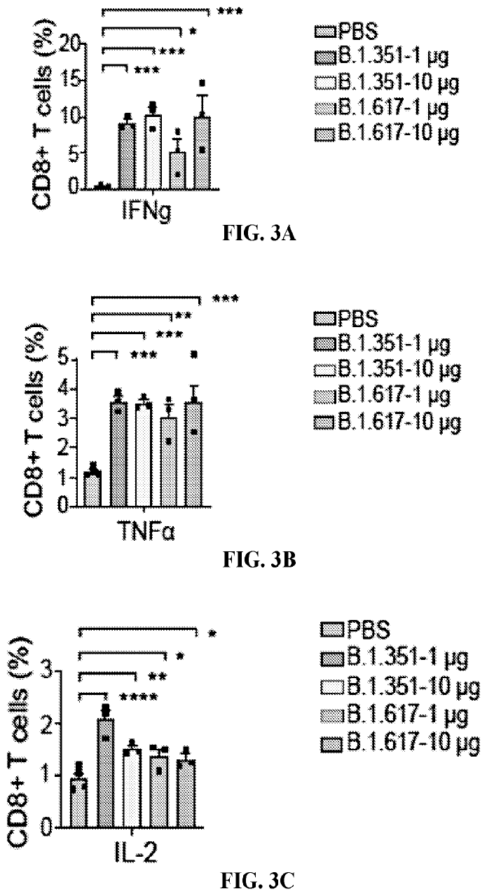

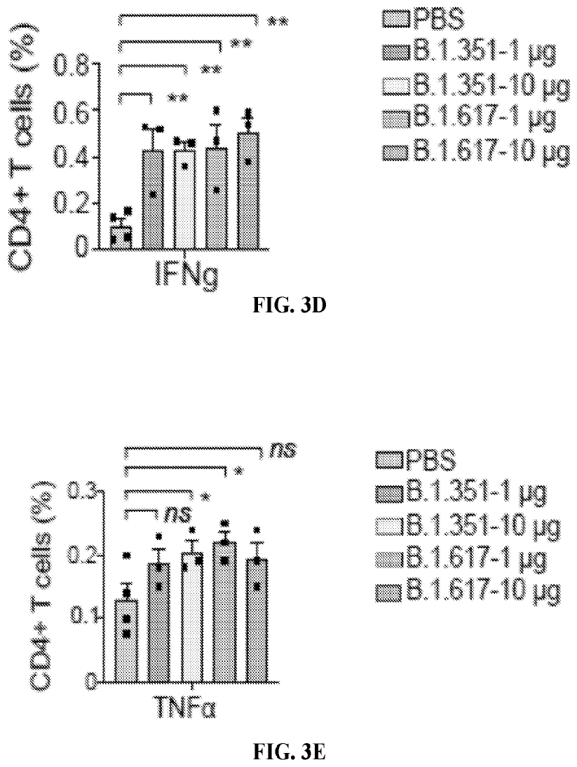

To evaluate the T cell response to the spike peptides, splenocytes were isolated from mouse spleens 40 days post vaccination and the antigen-specific CD4⁺ and CD8⁺ T cell responses to S peptide pools were determined by intracellular cytokine staining. Positive control PMA/ionomycin treatment and negative control no peptide groups were both validated. Both B.1.617-LNP-mRNA and B.1.351-LNP-mRNA, at low and high doses, induced potent reactive CD8⁺ T cell responses evidenced by cellular production of IFN-γ, TNF-α, and IL-2 (FIGS. 3A-3C). At both doses, both LNP-mRNAs also induced reactive CD4⁺ T cells that produce IFN-γ (FIG. 3D), minimal TNFα (FIG. 3E), but no IL-2, IL-4, or IL-5.

### Example 3: Single Cell, BCR and TCR Repertoire Profiling in Variant-Specific LNP-mRNA Vaccinated Mice Show Systemic Immune Activation and Responses after Vaccination

### Materials and Methods

Cell Isolation from Animals

For every mouse treated with either LNP-mRNA or PBS. Blood, spleens and draining lymph nodes were separately collected. Spleen and lymph node were homogenized gently and filtered with a 100 μm cell strainer (BD Falcon, Heidelberg, Germany). The cell suspension was centrifuged for 5 min with 400 g at 4° C. Erythrocytes were lysed briefly using ACK lysis buffer (Lonza) with 1 mL per spleen for 1˜2 mins before adding 10 mL PBS containing 2% FBS to restore iso-osmolarity. The single-cell suspensions were filtered through a 40 μm cell strainer (BD Falcon, Heidelberg, Germany).

### Bulk BCR and TCR Sequencing

Lymphocytes from blood, draining lymph node, and spleen of each mRNA-LNP vaccinated and control mice were collected as described above for mouse immunization and sample collection. mRNA of lymphocytes from three tissues were extracted using a commercial RNeasy® Plus Mini Kit (Qiagen). Bulk BCR and TCR were prepared using SMARTer Mouse BCR IgG H/K/L Profiling Kit and SMARTer Mouse TCR a/b profiling kit separately (Takara). Based on the extracted mRNA amount of each sample, the input RNA amounts for bulk BCR libraries were as follows: lymphocytes from blood (100 ng), lymphocytes from lymph node (1000 ng), and lymphocytes from spleen (1000 ng). The input RNA amounts for bulk TCR libraries were as follows: lymphocytes from blood (100 ng), lymphocytes from lymph node (500 ng), and lymphocytes from spleen (500 ng). All procedures followed the standard protocol of the manufacture. The pooled library was sequenced using MiSeq (Illumina) with 2*300 read length.

### Bulk VDJ Sequencing Data Analysis

Raw fastq files from bulk BCR and TCR sequencing were processed by MiXCR v2.1.5 to clonotypes. Paired-end reads were merged and aligned to reference genes for Homo sapiens species using the function: mixer align-s hs, Clones were assembled using the mixer assemble function, then exported for specific chains (TRB, TRA, IGH, IGL, IGK) using the mixer exportClones function. TCR-seq and BCR-seq data was subsequently analyzed using the immunarch v0.6.6 R package for clonality analyses and calculating diversity metrics such as the Chao1 estimator and Gini-Simpson index.

### Single Cell Profiling

Splenocytes were collected from mRNA-LNP vaccinated and control mice as described above for mouse immunization and sample collection, and normalized to 1000 cells/μL. Standard volumes of cell suspension were loaded to achieve targeted cell recovery to 10000 cells. The samples were subjected to 14 cycles of cDNA amplification. Following this, gene expression (GEX), TCR-enriched and BCR-enriched libraries were prepared according to the manufacturer's protocol (10× Genomics). All libraries were sequenced using a NovaSeq 6000 (Illumina) with 2*150 read length.

### Single Cell Transcriptomics Data Analysis for Immune Repertoire Profiling

Both standard established pipelines and custom scripts were used for processing and analyzing single cell GEX data. Illumina sequencing data was processed using the Cellranger v5.0.1 (10× Genomics) pipeline and aligned to the mm 10 reference. Cellranger outputs were then processed and analyzed using standard Seurat v. 4.0.2 workflow, including log normalization with scale factor 10,000, scaling and centering, principal components analyses, nearest-neighbor graph construction, clustering with the Louvain algorithm, uniform manifold approximation and projection (UMAP), differential gene expression, and generation of various visualizations. The following parameters were used: for the FindNeighbors function, dims=1:10; for FindClusters, resolution=0.6; for RunUMAP, dims=1:10; for FindAllMarkers, only.pos=TRUE, min.pct=0.25, logfc.threshold=0.25.

Assignment of immune cell type identity to clusters was performed manually based on expression of cell type specific markers. Custom scripts were used for cell proportion calculations and condition-specific analyses and statistics (e.g. Wilcoxon rank sum test). While cluster 5 cells were annotated as “CD8 T/NKT cell” as it was a mixed population, these cells were merged with the “CD8 T cell” annotation for proportion calculations after cells with greater than 1 expression for any of the following markers were removed: Klrb1, Klra6, Klra1, Zbtb16. Differential gene expression between conditions for various cell types were performed by the FindMarkers function with the parameters logfc.threshold=0.01 and min.pct=0.1. For T-cell specific analyses, cells associated with the following terms were taken as a subset and used for standard Seurat pipeline analyses as described above: “CD4 T cell”, “CD8 T/NKT cell”, “CD8 T cell”, “T cell-like.” For B-cell specific analyses, cells associated with the following terms were taken as a subset: “B cell”, “B cell-like”, “Progenitor B cell”, “Plasma cell.”

For functional annotation, differentially upregulated and downregulated genes with cutoff of adjusted p-value 0.05 were used for DAVID analysis. Genes associated with gene ontology terms “regulation of immune effector process” (GO:0002697), “immune response” (GO:0006955), “regulation of T cell activation” (GO:0050863), and “regulation of B cell activation” (GO:0050864) were used for generating annotation-associated heatmaps. Custom R scripts were used for generating various other figures.

### Single Cell VDJ Sequencing Data Analysis

Illumina sequencing data was processed using the Cellranger v5.0.1 (10× Genomics) pipeline and aligned to the mm10 VDJ reference. The filtered_contig_annotations output file was used as an input to immunarch v0.6.6 R package for calculating diversity metrics such as the Chao1 estimator and Gini-Simpson index. The clonotypes output file was used for analysis with custom scripts for clonality analyses and CDR3 distribution ring plots.

### Results

### Single Cell Immune Repertoire Mapping of B.1.617-LNP-mRNA and B.1.351-LNP-mRNA Vaccinated Animals

To gain insights on the global composition and transcriptional landscape of the immune cells, single cell transcriptomics (scRNA-seq) was performed on the spleen samples of B.1.351-LNP-mRNA and B.1.617-LNP-mRNA vaccinated animals. Using a total of 16 animals from 4 vaccination groups (B.1.351-LNP-mRNA and B.1.617-LNP-mRNA at both 1 μg and 10 μg dose groups each), plus a control group (PBS treated), the transcriptomes of a total of 90,152 single cells were sequenced and visualized on a Uniform Manifold Approximation and Projection (UMAP). Clustering was performed with Louvain algorithm, which identified 21 clusters from respective signatures of their differentially expressed genes. With the expression of a number of cell type specific markers, such as markers for pan-leukocytes (Ptprc/Cd45), B cells (Cd19, Cd22), plasma cells (Sdc1/Cd138), T cells (Cd3e, Cd4, Cd8a, Cd8b1, Trac/TCRa), natural killer (NK) cells (Ncr1, Klrb1c), dendritic cells/macrophages/monocytes (Cd11b/Itgam, Cd11c/Itgax, Adgre/F4/80, Mrc1, Gsr1), red blood cells (RBCs) (Hba-a1), and neutrophils (S100a8, Mmp9), cellular identities were assigned to the clusters, which included B cells (Cd19⁺), progenitor B cells (Csflr⁺;Cd19⁺), plasma cells (Igha⁺/Ighm⁺;Sdc1⁺;Cd19⁻), B cell-like cells (Cd19⁺;Ly6a⁺;), CD4 T cells (Cd3e⁺;Cd4⁺), CD8 T cells (Cd3e⁺;Cd8a⁺;Cd8b1⁺), NKT cells (Klra1⁺;Klra6⁺;Zbtb16⁺), DCs (Itgam⁺;Itgax⁺), macrophages (Itgam⁺;Csflr⁺;Adgre⁺;Mrcl⁺), monocytes (Itgam⁺;Csflr+;Gsr1⁺), neutrophils (S100a8⁺/S100a9⁺;Mmp9⁺), NK cells (Cd3e⁻;Ncr1⁺,Klrb1c⁺), and RBCs (Ptprc⁻;Hba-a1⁺). While Cluster 5 predominantly contained CD8 T cells, it also contained a small population of NKT cells that were not separated by the automatic clustering algorithm. The single cell transcriptomics provided a landscape of systemic immune cell populations and their respective gene expression (GEX) data in B.1.351-LNP-mRNA and B.1.617-LNP-mRNA vaccinated along with placebo control animals.

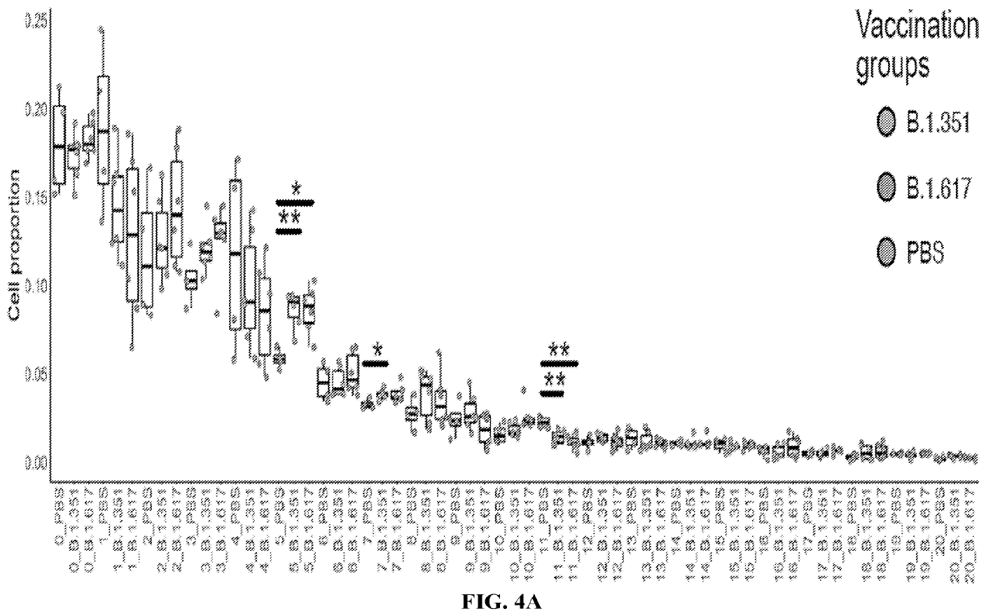

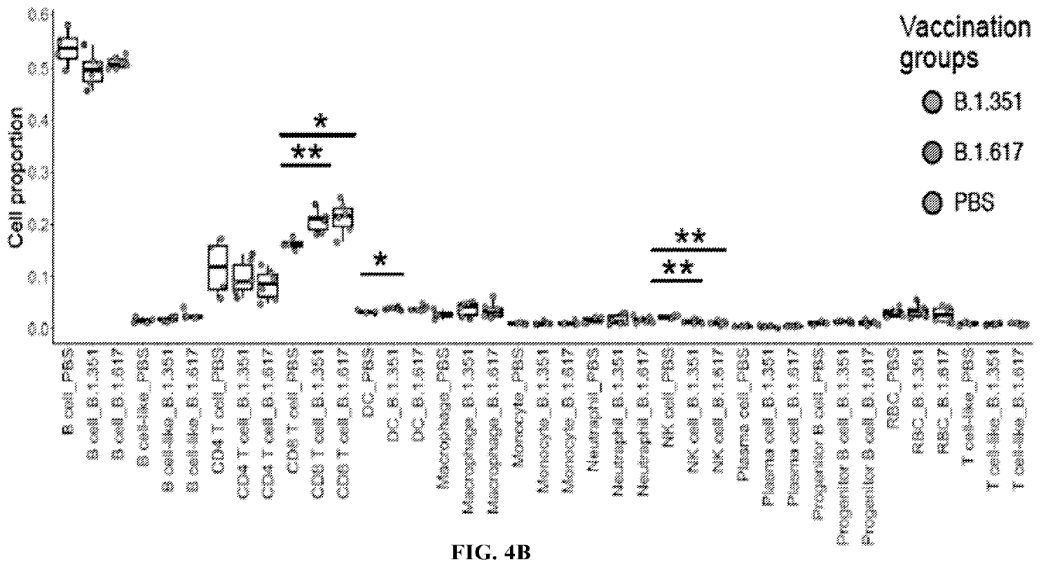

The systemic (spleen) immune cell compositions between placebo and vaccinated animals were then compared. Out of the 21 clusters, three showed significantly changed fractions in the total splenocytes upon vaccination as compared to placebo, including a significant increase in Cluster 5 (composed of CD8 T cells and NKT cells) for both B.1.351-LNP-mRNA and B.1.617-LNP-mRNA, a slight increase in Cluster 7 (DCs) for B.1.351-LNP-mRNA, and a slight decrease in Cluster 11 (NK cells) for both LNP-mRNAs (FIG. 4A). Analysis by summing all of the same cell types from different clusters validated this finding, revealing a significant increase in CD8 T cells (after excluding NKT cells) for both for B.1.351-LNP-mRNA and B.1.617-LNP-mRNA, a slight increase in DCs for B.1.351-LNP-mRNA, and a slight decrease in NK cells for both vaccination groups (FIG. 4B). It was observed that a fraction of Cluster 5's CD8 T cells also showed positive expression of Nkg7, Ifng, Gzma, and Cxcr3, representing a cluster of more activated T cells. The increase in CD8 T cells, especially the cluster of more activated T cells, is consistent with the strong CD8 T cell responses as detected by flow cytometry (FIGS. 3A-3C), indicative of systemic CD8 T cell responses upon vaccination with these variant-specific LNP-mRNAs.

### Gene Expression Signatures of B Cell and T Cell Populations of B. 1.617-LNP-mRNA and B.1.351-LNP-mRNA Vaccinated Animals

Because B and T cells are the cornerstones of adaptive immunity against SARS-CoV-2, the B cell sub-populations and T cell sub-populations were further investigated. Using the global clustering results with a number of B cell lineage markers, a total of 49,236 B cell-associated populations were identified from all samples and conditions of the 16 mice. Using unbiased clustering, these B cell sub-population cells were divided into 15 Clusters, although the largest 8 clusters were near each other in UMAP space and formed a “supercluster”.

Similarly, using the global clustering results with T cell lineage markers, a total of 28,099 T cell-associated populations were identified. Using unbiased clustering, these T sub-population cells were divided into 12 Clusters. Using more refined T cell markers, cells that represent sub-classes of T cells were detected, such as CD4 T cells (Cd3e⁺;Cd4⁺), CD8 T cells (Cd3e⁺;Cd8a⁺;Cd8b1⁺), regulatory T cells (Tregs) (Cd3e⁺;Cd4⁺;Foxp3⁺), Th1-like T helper cells (Th1s) (Cd3e⁺;Cd4⁺;Cxcr6⁺;Tbx21/Tbet⁺;Stat4*), Th2-like T helper cells (Th2s) (Cd3e⁺;Cd4⁺;Ccr4⁺;Il4ra⁺;Stat6⁺), Th17-like T helper cells (Th17s) (Tregs) (Cd3e⁺;Cd4⁺;Rorc⁺;Stat3⁺), and T follicular helper cells (Tfhs) (Cd3e⁺;Cd4⁺;Cd40lg⁺; Il4⁺;Il21⁺). As observed in various single cell datasets (Lindeboom, R. G. H., Regev, A. & Teichmann, S. A. Towards a Human Cell Atlas: Taking Notes from the Past. Trends Genet 37, 625-630, doi: 10.1016/j.tig.2021.03.007 (2021)), gating cellular populations by gene expression of these markers does not always produce clear cut populations defined by canonical immune markers using flow cytometry, possibly due to the differences between mRNA vs. surface protein expression, as well as the pleiotropic roles of various genes.

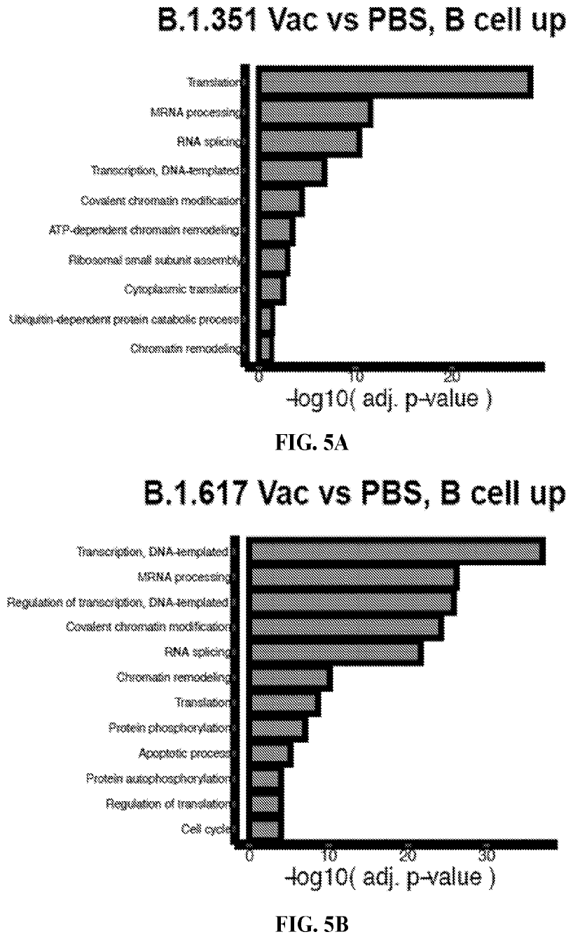

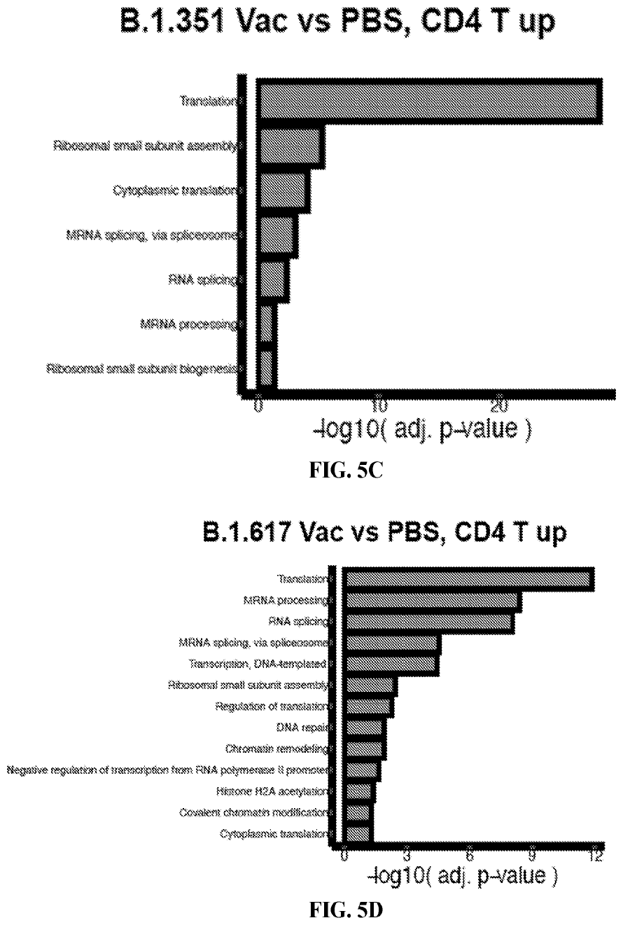

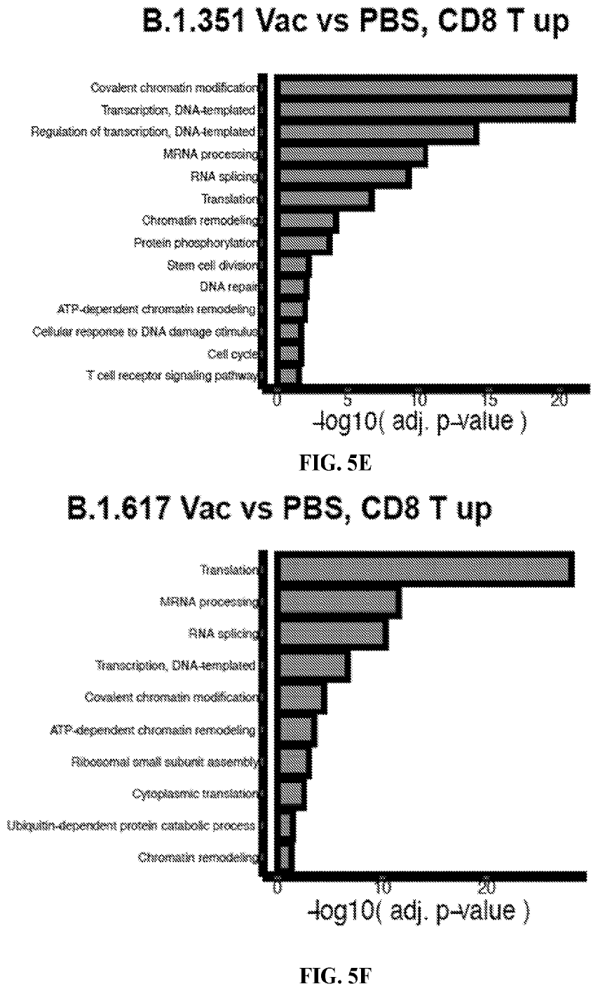

To examine the transcriptomic changes in the B and T cell sub-populations upon vaccination, differential expression (DE) analysis was performed in the matched sub-populations between PBS and B.1.351-LNP-mRNA, or B.1.617-LNP-mRNA, groups. Vaccination caused differential expression of genes in host B cells, CD4 T cells and CD8 T cells. The differentially expressed genes intersected with genes in B cell activation, immune effector, and immune response genes, such as Lyn, Cd22 and Btla. The differentially expressed genes in CD4 and CD8 T cells also intersected with genes in T cell activation, immune effector, and immune response genes, such as Cd40lg, Perforin/Prf1, Dhx36, Ddx17, Ddx21, Ccl5, Il18r1, Ptpn22 and Plcg1. Interestingly, the top upregulated expressed genes in B cells represent transcription and translation machineries, which is consistent between B.1.351-LNP-mRNA and B.1.617-LNP-mRNA vaccination groups (FIGS. 5A-5F). This strong signature was also observed in T cells (FIGS. 5A-5F), consistent with the phenomenon of active lymphocyte activation upon vaccination.

### TCR and BCR Diversity Mapping of B.1.617-LNP-mRNA and B.1.351-LNP-mRNA Vaccinated Animals

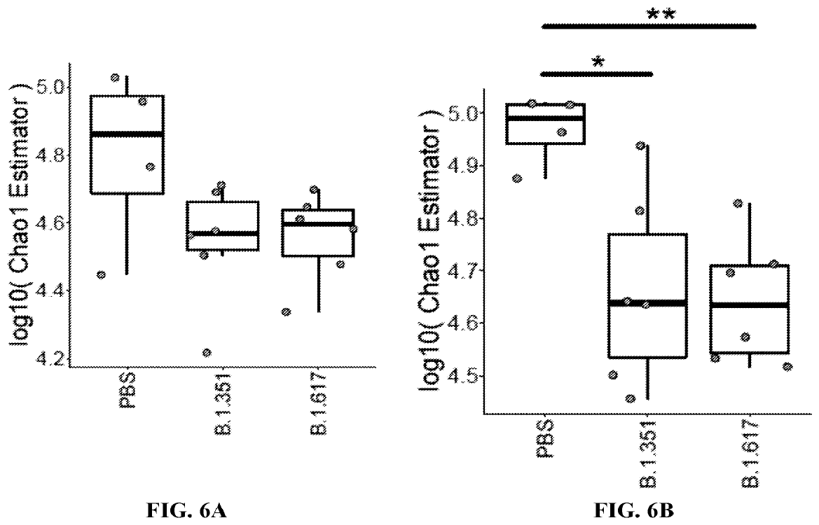

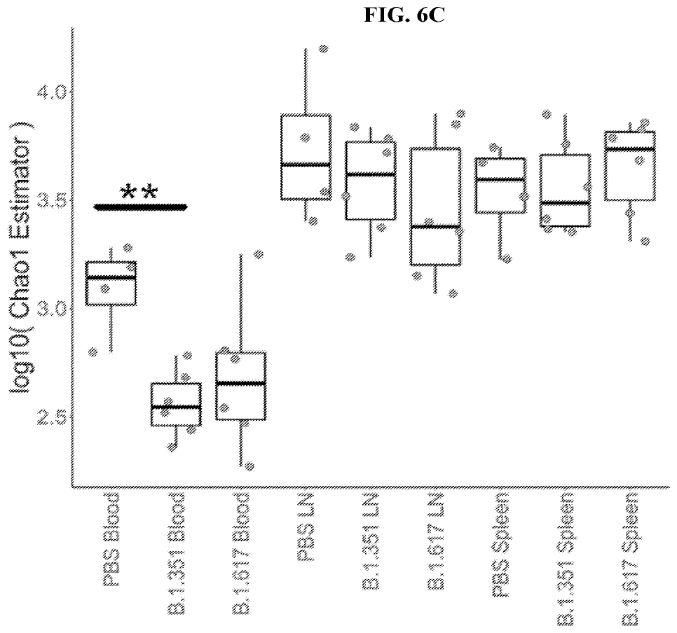

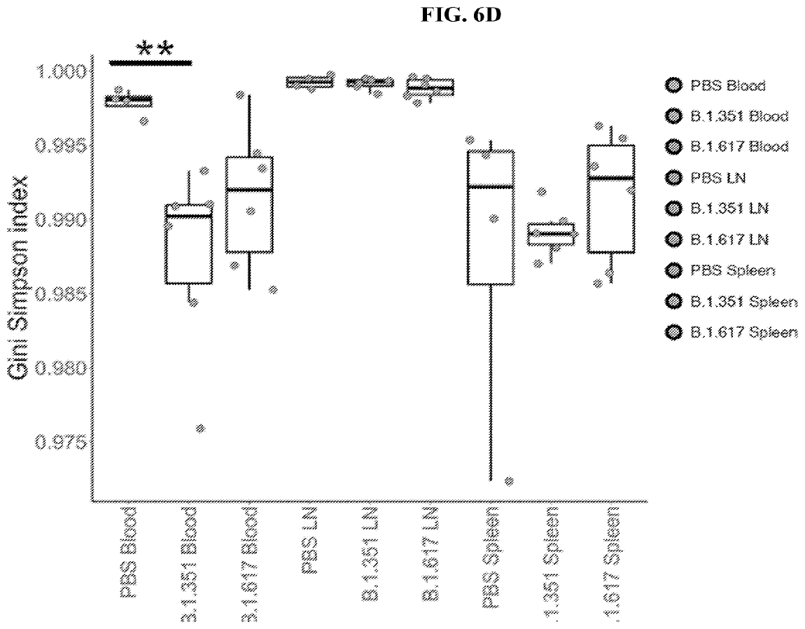

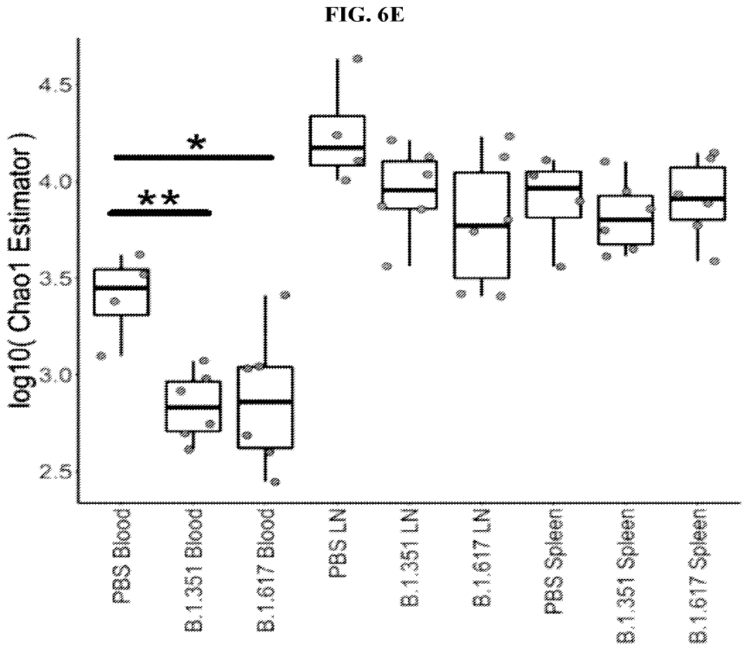

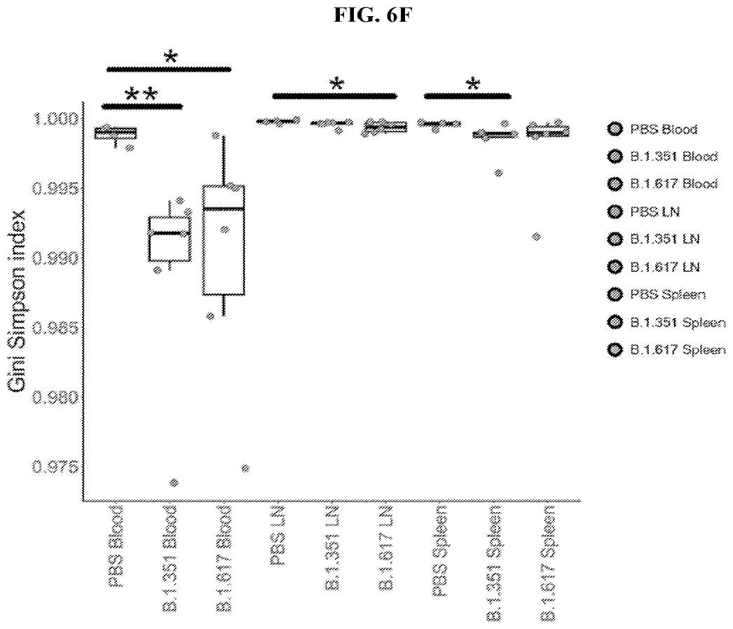

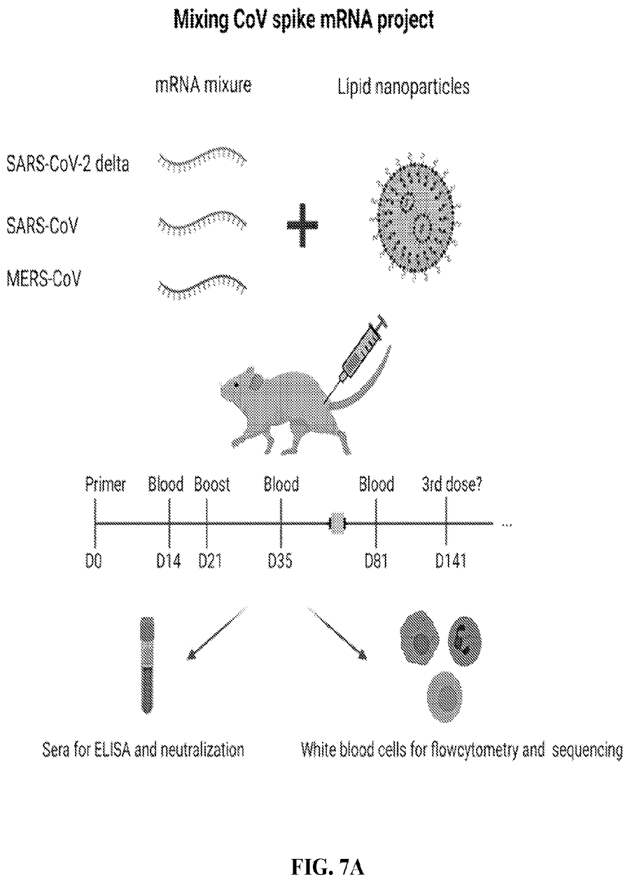

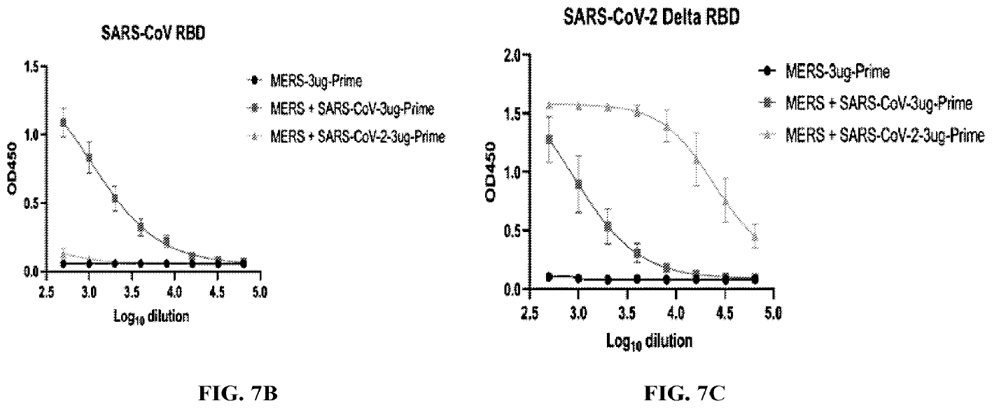

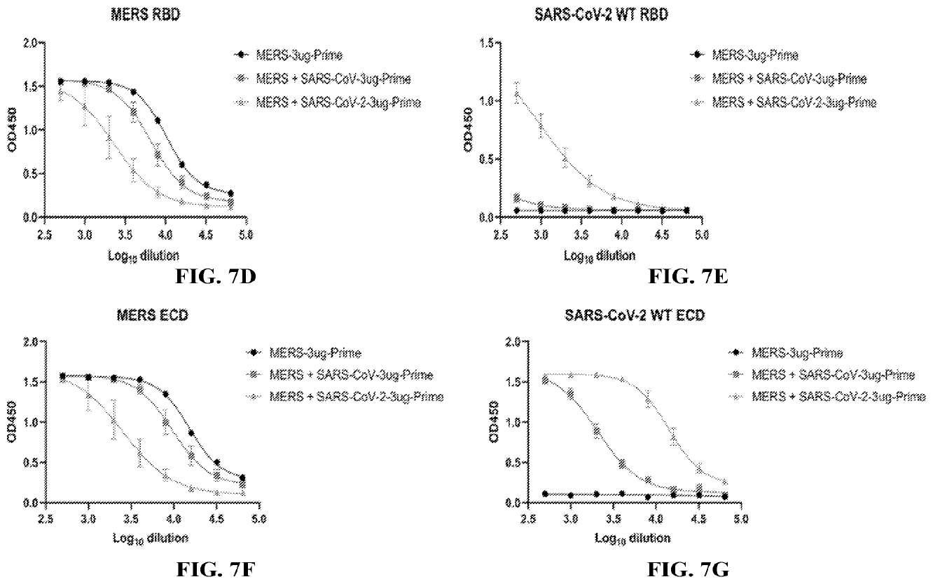

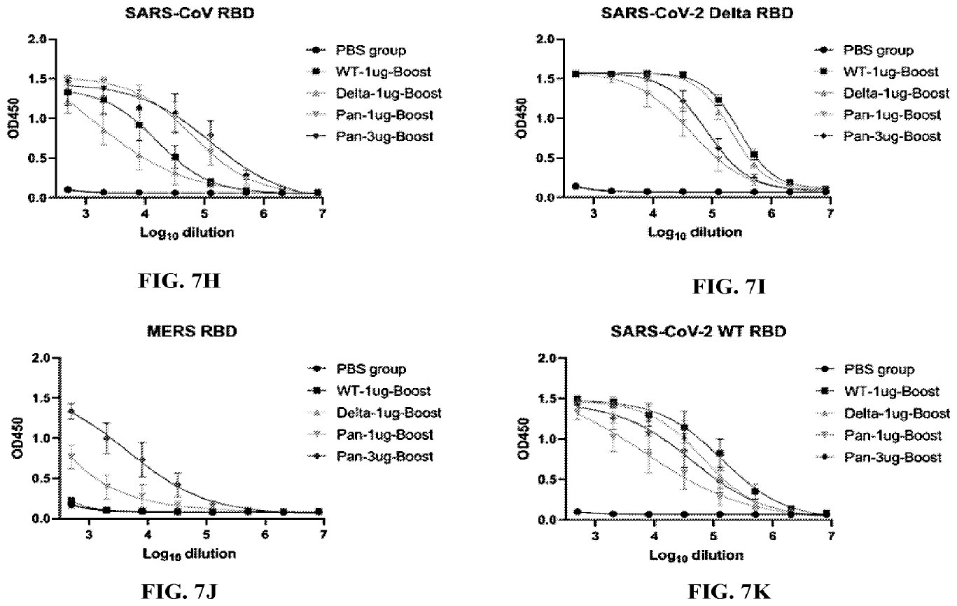

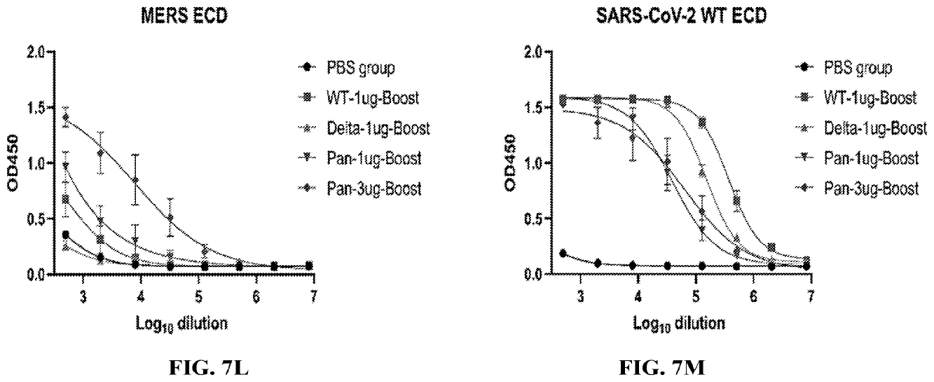

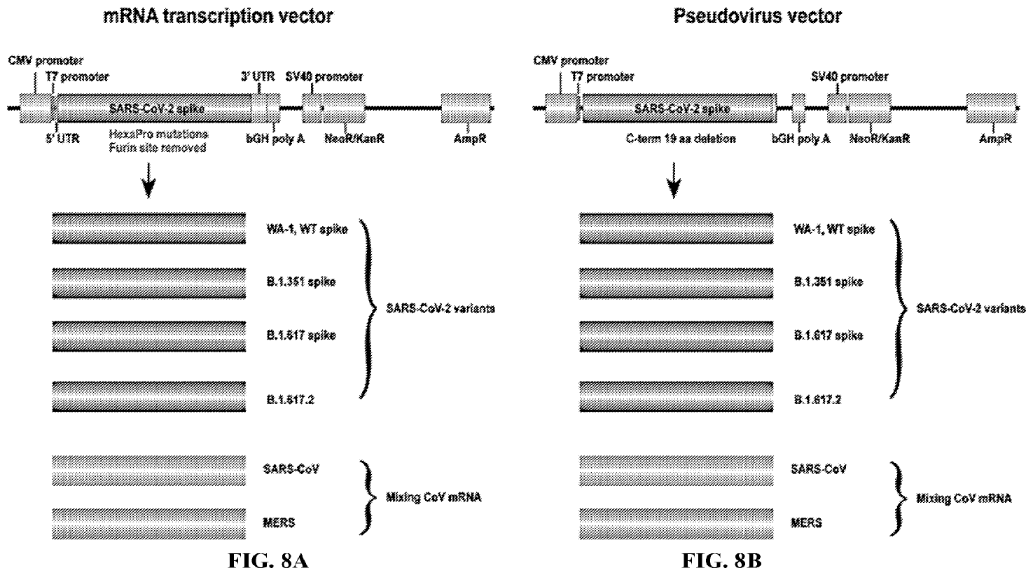

To reveal the B and T cell clonal diversity and influence by vaccination, VDJ repertoire mapping and clonal analyses of B cell and T cell populations of B.1.351-LNP-mRNA and B.1.617-LNP-mRNA vaccinated animals was performed. Both single cell BCR sequencing (scBCR-seq) and single cell TCR sequencing (scTCR-seq) were performed on the spleen samples of all groups (4 vaccination groups and a PBS group, n=16 mice total). A total of 47,463 single B cells and 25,228 single T cells were sequenced. Clonal composition showed the BCR repertoire in the single cell BCR-seq dataset, revealing a trend towards decreased clonal diversity (FIG. 6A). The clonal composition of single cell TCR showed a significant decrease in clonal diversity (FIG. 6B). This phenomenon is consistent with the clonal expansion of stimulated lymphocytes upon vaccination, which is more pronounced in the scTCR-seq data.

To further validate the observations, bulk BCR-seq and bulk TCR-seq were also performed for all these mice on additional tissue samples, including spleen, peripheral blood (PB) and lymph node (LN). The bulk BCR-seq and TCR-seq data revealed systematic clonality maps of the spleen, PB and LN samples of the variant-specific LNP-mRNA vaccinated along with placebo treated animals (FIGS. 6C-6F). The bulk TCR-seq results, from both TCR alpha and beta chains (TRA and TRB), validated the observation of decreased clonal diversity from single cell VDJ profiling (FIGS. 6C-6F), again consistent with the notion of clonal expansion of a small number of clones. The clonal diversity decrease/clonal expansion effect was strongest in the PB samples that capture circulating T cells (FIGS. 6C-6F). These data together unveiled BCR and TCR repertoire clonality, diversity and respective shifts in variant-specific LNP-mRNA vaccinated animals as compared to placebo-treated.

### Selected Discussion

Although efficacious COVID-19 mRNA vaccines have been deployed globally, the rapid spread of SARS-CoV-2 VoCs, with higher contagiousness as well as resistance to therapies and vaccines, demands evaluation of next-generation COVID-19 vaccines specifically targeting these evolving VoCs. Mounting evidence has indicated that the B.1.351 and B.1.617 lineage variants of SARS-CoV-2 possesses much stronger immune escape capability than the original wildtype virus. The lower neutralizing titers in fully vaccinated patients were found associated with breakthrough infections. It has been speculated that the waning immunity from early vaccination and emergence of more virulent SARS-CoV-2 variants may lead to reduction in vaccine protection and increase of breakthrough infections. It has been reported that mRNA vaccines' efficacy against B.1.351 and B.1.617.2 dropped significantly. Moreover, for individuals receiving only a single dose of vaccine, the protective efficacy can be dramatically lower. It is worth noting that efficacy value and definition may vary from study to study, which have been conducted in different regions and populations. In view of these facts, this study evaluated next-generation mRNA vaccine candidates encoding the B.1.351 and B.1.617 spike as antigens.

This study characterized the titers and cross-reactivity of sera from mice vaccinated with WT-, B.1.351- or B.1.617-LNP-mRNAs to all three WT, B.1.351 and B. 1.617 spike antigens and pseudoviruses. In agreement with findings in patients' sera, it was observed that the neutralizing titers of WT vaccine sera were several fold lower against the two variants of concern than against WT pseudovirus. The B.1.617-LNP-mRNA vaccinated sera also showed particularly strong neutralization activity against its cognate B.1.617 pseudovirus, while the B.1.351-LNP-mRNA showed similar neutralization activity against all three pseudoviruses. It is worth noting that all three forms of vaccine candidates can induce potent B and T cell responses to WT as well as the two VoCs' spikes.

The T cell-biased immune response is important for antiviral immunity, and therefore, the efficacy and safety of viral vaccines. To evaluate the Th1 and Th2 immune response by the variant vaccines, intracellular staining of Th1 and Th2 cytokines was performed in splenocytes. After stimulation with peptide pools covering the entire S protein, the splenocytes from three mRNA vaccine groups produced more hallmark Th1 cytokine IFN-γ in both CD4⁺ and CD8⁺ T cells than those from PBS group. The flow cytometry data indicated that the two variant vaccine candidates induced strong Th1-biased immune responses, just like the WT vaccine, of which a Th1 response had been observed by previous studies in animal models (Corbett, K. S. et al. SARS-CoV-2 mRNA vaccine design enabled by prototype pathogen preparedness. Nature 586, 567-571, doi: 10.1038/s41586-020-2622-0 (2020)).

Single cell sequencing is a powerful technology for immune and gene expression profiling, which has been utilized for mapping immune responses to COVID-19 infection. To gain insights on the transcriptional landscape of the immune cells and clonal repertoire changes specifically in B and T cells, single cell transcriptomics as well as BCR and TCR repertoire sequencing were performed. The single cell transcriptomics data revealed a systematic landscape of immune cell populations in B.1.351-LNP-mRNA and B.1.617-LNP-mRNA vaccinated animals. The repertoires and associated global gene expression status of the immune populations including B cells, T cells, and innate immune cells were mapped out. From the overall splenocyte population, a distinct and significant increase in the CD8 T cell populations in vaccinated animals was observed. Interestingly, differential expression between vaccinated and placebo-treated animals showed a strong signature of increased expression of transcriptional and translational machinery in both B and T cells. These phenomena potentially reflect the active proliferation and immune responses in these lymphocytes.

BCR and TCR sequencing are efficient tools for mapping of clonal repertoire diversity, which has been utilized for sequencing COVID-19 patients. BCR-seq and TCR-seq unveiled the diversity and clonality and respective shifts in variant-specific LNP-mRNA inoculated animals as compared to placebo-treated. The decrease in VDJ clonal diversity, along with clonal expansion of a small number of clones, were observed in vaccinated animals as compared to placebo group. Vaccinated animals from both B.1.351-LNP-mRNA and B.1.617-LNP-mRNA groups showed clonal TCR expansion, especially pronounced in peripheral blood samples. The induction of diverse and expanding clones is a signature of vaccine induced protective immunity.

This study provides direct assessment of in vivo immune responses to vaccination using LNP-mRNAs encoding specific SARS-CoV-2 variant spikes in pre-clinical animal models. The single cell and bulk VDJ repertoire mapping also provide unbiased datasets and robust systems immunology of SARS-CoV-2 vaccination by LNP-mRNA specifically encoding B.1.351 and B.1.617 spikes. These data provide valuable insights for the development of the next-generation COVID-19 vaccines against the SARS-CoV-2 pathogen and especially its emerging variants of concern. Furthermore, the multiplexed and chimeric vaccines provide compositions and methods for vaccination protection against other pathogenic coronaviruses such as MERS-CoV and SARS-CoV, while the vaccine designs can be broadly applicable to other emerging pathogenic coronaviruses.

### Example 4: AI/ML-Powered Vaccine Antigen Design, and Development of AAV and Virus-Like Particle (VLP) Vaccine

### Materials and Methods:

### Artificial Intelligence (AI)/Machine Learning (ML)

Artificial intelligence (AI)/machine learning (ML) based vaccine design pipelines were developed, in order to identify the pathogenic and/or immunogenic regions in the coronavirus genomes, and to enhance vaccine antigen design for next-gen coronavirus vaccine candidates.

A total of 3,665 complete nucleotide genomes of the “Coronaviridae” family were downloaded from the Virus Pathogen Database and Analysis Resource (ViPR) database (Pickett et al., 2012) to be used for machine learning algorithm training. Genbank accession MN908947 was used as the reference SARS-CoV-2 sequence for downstream analyses. Coronavirus protein sequences for spike protein (YP_009755834, ACN89696, ABD75577, QIQ54048, QHR63300, QHD43416, QDF43825, ATO98157, AAP13441, ASO66810, ALD51904, AYF53093, AKG92640, ALA50214, AFD98757, AJP67426, AHX26163, AVM80492) and ORF1ab (QIT08254, QJE38280, QJD07686, QHR63299, QIA48640, QDF43824, AAP13442, QCC20711, AJD81438, AHE78095, ATP66760, ABD75543, YP_009019180, AVM80693, AFU92121, AFD98805, APZ73768, ATP66783, YP 002308496) used for evolutionary analyses were obtained from the NCBI Virus community portal. Amino acid sequences for SARS-CoV-2 were obtained from translations from reference sequence NC_045512 (equivalent to MN908947). FASTA sequences for S protein (YP_009724390), E protein (YP_009724392), M protein (YP_009724393), N protein (YP_009724397), NSP3 (YP_009742610), NSP5 (YP_009742612), NSP8 (YP_009742615), NSP9 (YP_009742616), and NSP12 (YP_009725307) were obtained from the NCBI Protein database and were used for downstream evolutionary and immune epitope analyses.

### Pre-Processing

Sequences were aligned with MAFFT (Katoh et al., 2019) version 7 with the—auto strategy. Degenerate IUPAC base symbols that represent multiple bases were converted to “N” and ultimately masked prior to training algorithms. Six bp-wide sliding windows with 1 bp shifts were generated across every position in the alignment for a total of 100,835 alignment-tiled windows. Genetic features including nucleotides and gaps for a given window were converted to binary vector representations using LabelEncoder and OneHotEncoder from the Python scikit-learn library (Pedregosa et al., 2011), for integer encoding of labels and one-hot encoding respectively. Additional Python libraries used include BioPython (Cock et al., 2009), NumPy (van der Walt et al., 2011), and pandas (Mckinney, 2010).

### Principal Components Analysis

Dimensionality reduction of encoded whole coronavirus genomes was performed primarily using R scripts. The MSA was converted to cell-based representations in a CSV file, followed by one hot encoding, PCA, and visualization with metadata labelling. One hot encoding with performed with the “mltools” R package and PCA was performed with the “prcomp” R functio

### Training and Evaluating Machine Learning Base Models

Genome metadata was converted to binary vector classifications with “1” representing predictor class genomes depending on classification strategy and “0” representing all other genomes. Three different classification strategies were used: (1) predictor class containing coronavirus samples infecting human hosts, (2) predictor class containing all SARS-CoV-2, SARS-CoV, and MERS-CoV samples, and (3) predictor class containing SARS-CoV-2, SARS-CoV, and MERS-CoV samples specifically infecting human hosts. Five supervised learning classifiers from scikit-learn were used for training and evaluation, with seeds set at 17 for algorithms that use a random number generator. Support vector classifiers (SVC) were trained with a linear kernel and regularization parameter of 1.0; random forest (RF) classifiers were trained with 100 estimators; Bernoulli Naïve Bayes (BNB) were trained with alpha of 1.0 with the “fit_prior” parameter set as true to learn class prior probabilities; multi-layer perceptron (MLPC) classifiers were trained with “lbfgs” solver, alpha of 1e-5, 5 neurons in the first hidden layer, and 2 neurons in the second hidden layer; gradient boosting classifiers (GBC) were trained with “deviance” loss function, learning rate of 0.1, and 100 estimators. All estimators were trained and evaluated with stratified 5-fold cross-validation on each window, using 80% of the data for training and 20% of the data for validation.

### Statistical Hypothesis Test-Based Meta-Model

Accuracy scores obtained from machine learning base models were used as a proxy for “learned, predictive information content” to determine coronavirus pathogenicity (COPA) scores using a statistical hypothesis test-based meta-model. First, Shannon entropy values were calculated for each window across the alignment. Windows with minimal entropy values (n=10,383), typically found in highly gapped regions, were used to define a biologically meaningful control group; i.e., it was hypothesized that windows with low information content in highly gapped regions should not be predictive of coronavirus pathogenicity and should have minimal discriminative value. For each position across the alignment (100,840 positions), scores associated with windows that overlap with the position (typically ˜six windows) were pooled and tested to see if statistically significantly different from the minimal entropy control group using the nonparametric two-sided Wilcoxon rank-sum test. For the main NT-COPA score calculations and evolution-based analyses, all scores across the three classification strategies were used for testing; in supplemental analyses, scores for individual classification strategies were used separately. This procedure was performed across the alignment, and p-values were adjusted for multiple comparisons using the Benjamini & Hochberg procedure. P-values were transformed to nucleotide resolution coronavirus pathogenicity scores by negative log base 10 (also referred to as NT-COPA scores). Amino acid resolution scores were obtained by averaging the NT-COPA scores for a given residue's codon (referred to simply as COPA scores).

### Kernel Regression Smoothing for Hotspot Peak Identification

For a systematic strategy to identify pathogenicity hotspots across the SARS-CoV-2 genome using COPA scores, kernel regression smoothing combined with local maxima identification was used. For each position across the alignment, the Nadaraya-Watson kernel regression estimate was determined using the ksmooth function in R with a “normal” kernel and various bandwidth sizes. Peaks highlighted in this study are primarily based on estimates calculated with bandwidth size of 3. Local peaks were determined from kernel regression estimates using the “findpeaks” function with nups parameter set at 2, from the “pracma” R package.

### Evolutionary Analyses

Protein sequences used for evolutionary analyses were aligned using MAFFT version 7 with the “L-INS-i” strategy (Katoh et al., 2019). Alignments were visualized using Jalview 2.11.1.0 (Waterhouse et al., 2009). Phylogenic analyses were performed using MEGA10.1.8 software (Kumar et al., 2018). Phylogeny trees were generated with the Maximum Likelihood statistical method, Jones-Taylor-Thornton (JTT) substitution model, uniform rates among sites, use of all sites, Nearest-Neighbor-Interchange (NNI) heuristic method, and default NJ/BioNJ initial tree. For spike protein analysis, all obtained sequences were used for alignment and phylogeny. For NSP12 analysis, all obtained ORF1ab sequences and reference SARS-CoV-2 NSP12 (YP_009725307) were used for alignment, but only ORF1ab sequences were used for phylogeny.

For large scale phylogenetic analysis, efficient tree inference on the full genome set multiple sequence alignment was performed using IQ-TREE version 2.0.6 (Minh et al., 2020) with the GTR+F+R10 model, which was selected automatically using ModelFinder (Kalyaanamoorthy et al., 2017). Circular phylogenetic trees were then generated for visualization and labelled using FigTree v1.4.4.

### Structural Analyses

The crystal structure of SARS-CoV-2 spike receptor-binding domain bound with ACE2 was obtained from Protein Data Bank (PDB) with accession code 6M0J (Lan et al., 2020). The cryo-EM structure of the SARS-CoV-2 NSP12-NSP7-NSP8 complex bound to the template-primer RNA and the triphosphate form of remdesivir (RTP) was obtained from PDB with accession code 7BV2 (Yin et al., 2020). The crystal structure of SARS-CoV spike RBD bound with ACE2 was obtained from PDB with accession code 2AJF (Li et al., 2005). Molecular graphics and analyses including mapping of COPA scores onto structures were performed with UCSF ChimeraX version 0.94 (Goddard et al., 2018).

### B Cell Epitope Analysis

FASTA sequences for reference SARS-CoV-2 structural proteins were used to predict B cell epitopes. Linear B-cell epitopes probability scores were obtained using BepiPred-2.0 (Jespersen et al., 2017). “Consensus Regions” were defined as amino acid residues with epitope scores>0.5 and COPA scores>8. Hypergeometric test of overlap of high COPA score (>8) and high epitope score (>0.5) residues was performed to determine the statistical significance of consensus regions. “Compound Regions” were identified using k-means clustering. Briefly, the R function “kmeans” was run with variable number of clusters and nstart parameter 25 on a dataset containing residue position, epitope score, and COPA score. Residues were marked as compound regions if they belonged to clusters with epitope score centers>0.5 and COPA score centers>8. Flagged residues that did not belong to a contiguous run of amino acids≥5 residues were filtered out.

### T Cell Epitope Analysis

FASTA sequences for reference SARS-CoV-2 structural proteins and select nonstructural proteins were used to predict T cell epitopes. Prediction of peptides binding to MHC class I and class II molecules was then performed using TepiTool (Paul et al., 2016) from the Immune Epitope Database (IEDB) Analysis Resource. MHC-I binder predictions were made for the “Human” host species and the 27 most frequent A & B alleles in the global population. Default settings for low number of peptides (only 9mer peptides), IEDB recommended prediction method, and predicted percentile rank cutoff≤1.0 were used for peptide selection. MHC-II binder predictions were made for the “Human” host species using the “7-allele method” (median of percentile ranks from DRB1*03:01, DRB1*07:01, DRB1*15:01, DRB3*01:01, DRB3*02:02, DRB4*01:01, DRB5*01:01). Median consensus percentile rank≤20.0 was used for peptide selection. Pathogenicity associated peaks within the proteins with NT-COPA scores greater than 8 were then mapped to the predicted peptides for prioritization.

### Vaccine Antigen Design

Based on the analyses above using coronavirus genomics and AI/ML, the identified regions, with considerations on (i) pathogenicity, (ii) B cell epitopes and (iii) T cell epitopes, were fed into the antigen design. These resulting vaccine antigen designs were synthesized as gBlocks, and cloned into vaccine vectors, e.g., mRNA vector or viral vector.

Amino acid sequences of the designed antigens are as follows:

pJP61 antigen
(SEQ ID NO: 18)
MVNCTEVPVAIHADQLTPTWRVYSTGSNVFQTAAYNGTITVEELKKLLEQ
AAYYVYSRVKNLNSSRVPDLLVAAYNGPQNQRNAPRITFGGPSDSTGSNQ
NGERSGARSKQRRPQGLPNNAAYYQTQTNSPRRARSVASQSIIAYTMSLG
AENSVAYSNNSIAAYEVRQIAPGQTGKIADYNYKAAYKLGASQRVAGDSG
FAAYSRYRIGNYKAAYDAYKTFPPTEPKKDKKKKADETQALPQRQKKQQT
VTLLAAYGKQGNFKNLREFVEKNIDGYFKIYSKHAAYHHHHHH
pJP62 antigen
(SEQ ID NO: 19)
MFVFLVLLPLAAYNLIDSYFVVAAYLLLDRLNQLAAYKLLEQWNLVAAYV
LLFLAFVVAAYLLSAGIFGAAAYVLAWLYAAVAAYALWEIQQVVAAYTMA
DLVYALAAYFLWLLWPVTAAYFLAFVVFLLAAYFLKKDAPYIAAYFLNRF
TTTLAAYLLMPILTLTAAYKIADYNYKLAAYFTISVTTEIAAYFVLAAVY
RIAAYYLATALLTLAAYHHHHHH
pJP63 antigen
(SEQ ID NO: 20)
MTQDLFLPFFSNVTWFAAYDGVPFVVSTGYHFREAAYDDQIGYYRRATRR
IRAAYWICLLQFAYANRNRFAAYSVLLFLAFVVFLLVTAAYNEFACVVAD
AVIKTLAAYDLLIRKSNHNFLVQAAAYAIASEFSSLPSYAAFAAYKYLYF
IKGLNNLNRGAAYLLLLDRLNQLESKMSAAYMWLSYFIASFRLFARAAYE
FVFKNIDGYFKIYSAAYFHQKLLKSIAATRGAAAYFLLVTLAILTALRLC
AAYSGYLKLTDNVYIKNAAAYPLNIIPLTTAAKLMVAAYHHHHHH
pJP64 antigen
(SEQ ID NO: 21)
MTPINLVRDLPQGESALEPLVAAYLNQLESKMSGKGQQQQGQTVTKKSAA
EASKKPRQKRTATKAYNVTQAFGRRGPEQTQAAYYLQPRTFLLAAYLPFF
SNVTWFHAIHVAAYDAALALLLLDRLNQLAAYFLFLTWICLAAYNRNRFL
YIIKLIFLWAAYSEETGTLIVNSVLLFAAYTNGTKRFDNPVLPFNAAYHG
KEDLKFPRGQGVPINTNSSPDDQIGYYRRATRRIAAYSVLLELAFVAAYR
FPNITNLCPFGEVFNATRFAAYTLACFVLAAVYRINWAAYFLLVTLAILA
AYHHHHHH
pJP65 antigen
(SEQ ID NO: 22)
MCNDPFLGVYYHKNNKSWMESEFRVAAYDLDDFSKQLQQSMSSADSTAAY
SLLIVNNATNVVIKVAAYNLLKDCPAVAAYFHQKLLKSIAATRGAAAYVV
QTIEVNSFSGYLKAAYYKFVRIQPGQTFSVLAAYAKLMVVIPDYNTYKNA
AYVLKLKVDTANPKTPKAAYTGIAVEQDKNTQEVFAQVKQIYKTPPIKDF
GGFNFSQILPDPSKPSKRSFIEDLLFNKVTLADAGFIKQYGDCLAAYRGM
VLGSLAATVRLQAAYALAYYNTTKAAYPTVVVNAANVYLKHGAAYHHHHH
H

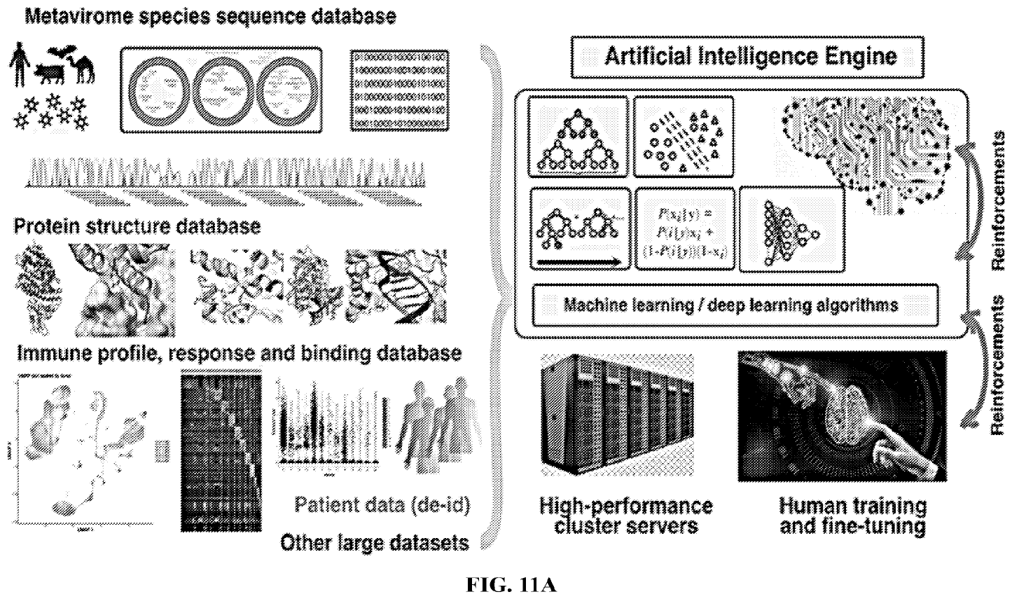

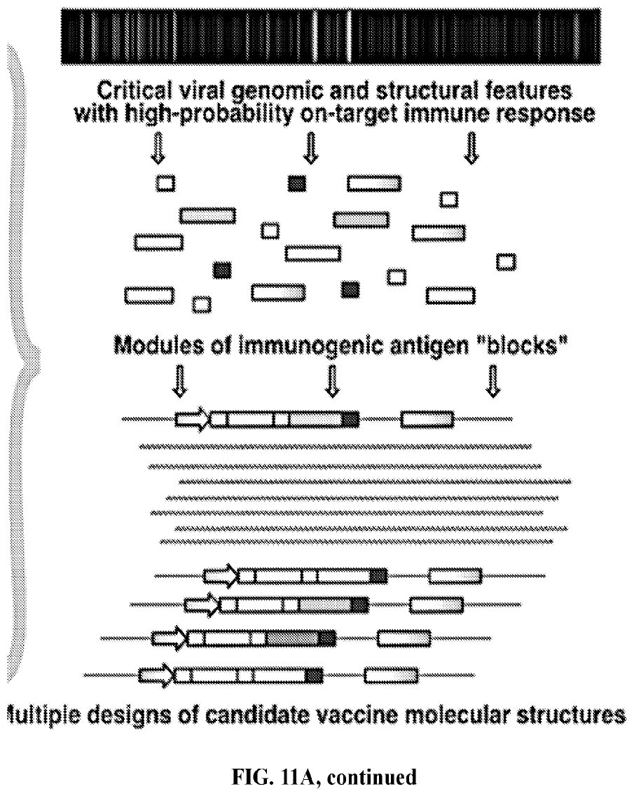

In an example, the antigens were cloned into AAV vectors, packaged, and tittered by qPCR and functional transduction tests using fluorescence scope and FACS (see FIGS. 11B-11C). The reporter AAV-GFP was tested in parallel to validate the functionality of the AAV vectors.

### Results

Artificial intelligence (AI)/machine learning (ML) based vaccine design pipelines were developed. The AI/ML algorithms identify the pathogenic and/or immunogenic regions in the coronavirus genomes, to enhance vaccine antigen design for next-gen coronavirus vaccine candidates. In this setting, the vaccine antigen design was performed using AI/ML-powered algorithms. The antigens were created as synthetic constructs, sub-cloned into expression vectors, such as AAV or VLP (these antigens can also be used in mRNA vaccine formulation). Results of qPCR titration of representative AAV vaccine candidate preps showed that these AAVs encoding the vaccine antigens can be produced. Results of imaging or flow-based functional titration also showed the positivity of packaged AAVs (see FIGS. 11A-11D).

Vaccine Cellular Assay System was Generated and AAV/VLP Vaccine Candidates were Tested.

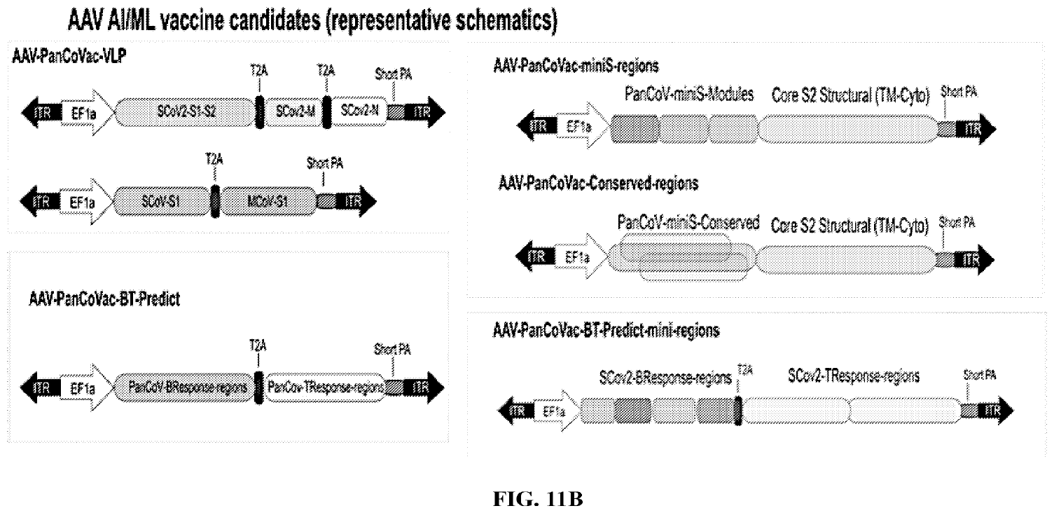

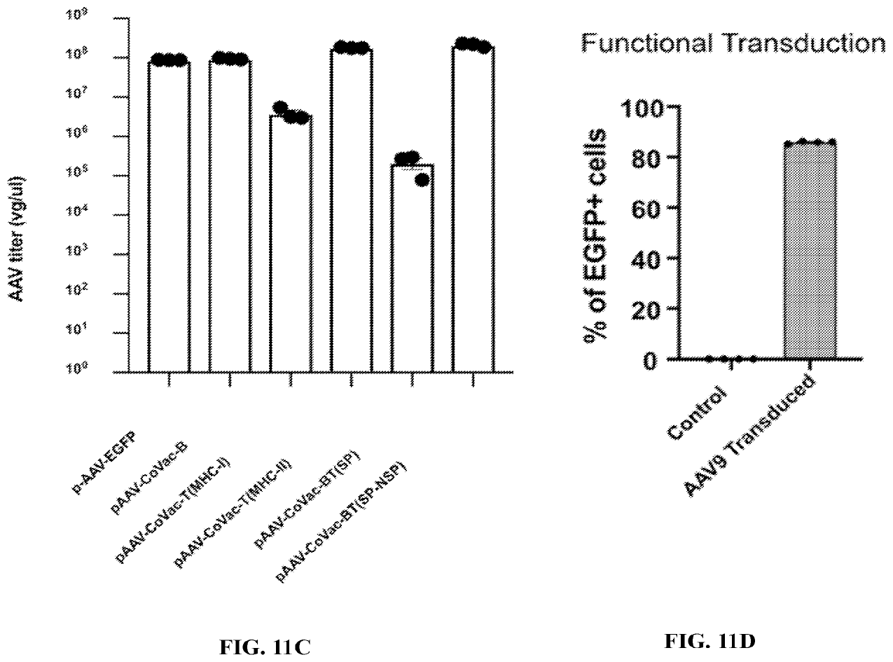

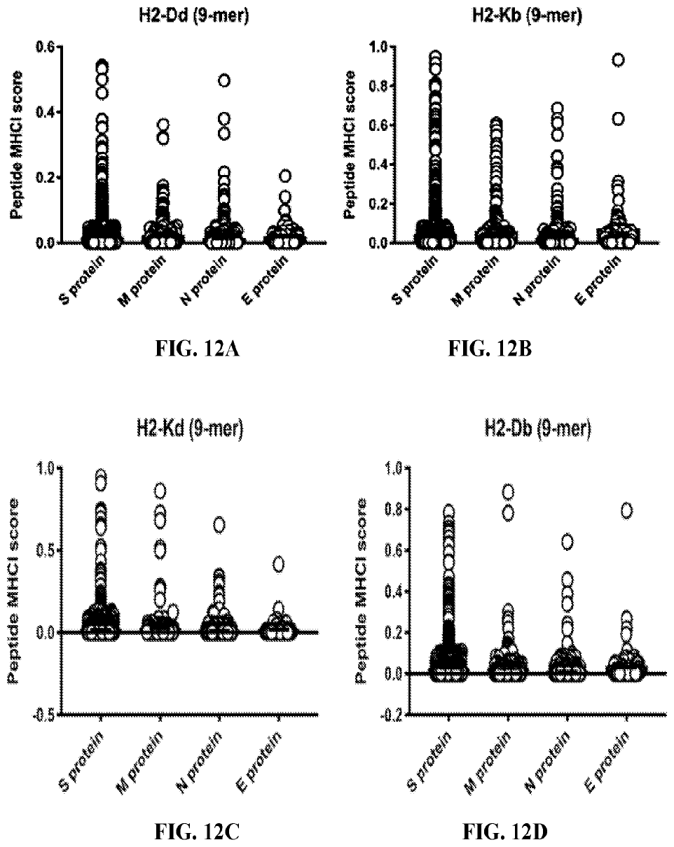

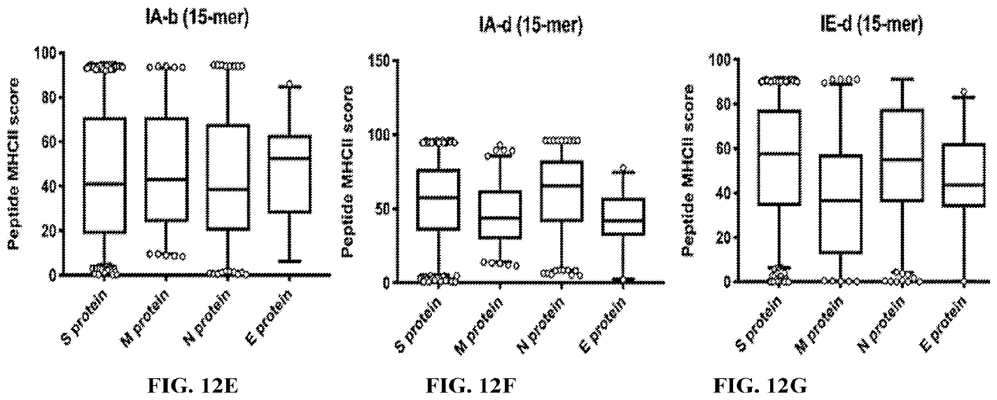

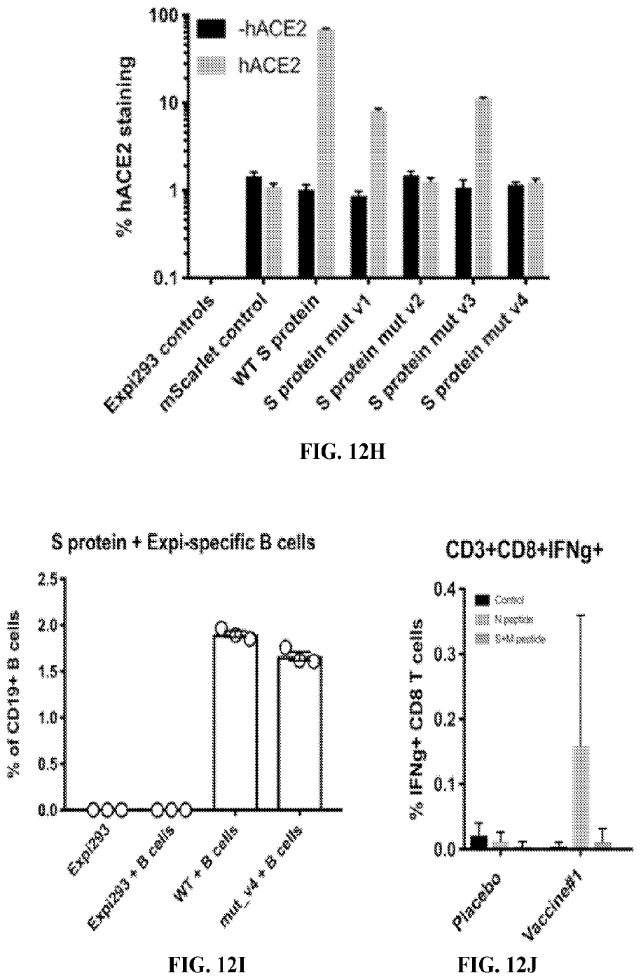

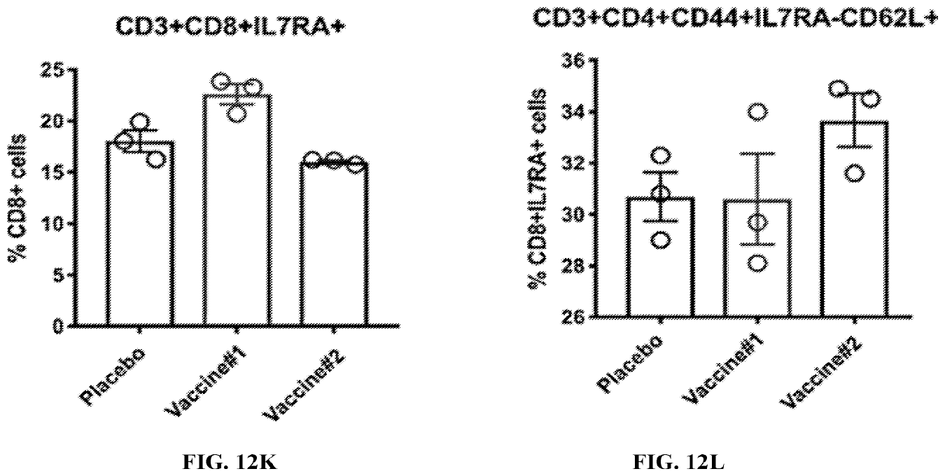

MHC-I and MHC-II scores of antigens were predicted similar to above. A cellular hACE2 system was established for antigen testing. The hACE2 system is a quantitative immunostaining approach for estimating reactive cells to Spike antigen. FACS-based quantification of Spike-specific B cell populations was performed on the splenocyte samples of the AAV vaccine injected animals in vivo, showing positive response of antigen-specific B cells. FACS-based quantification of vaccine-induced antigen-specific IFNg+CD8 T cell populations in vivo also showed positive antigen-specific T cells. FACS-based quantification of IL7RA+ and CD44+IL7RA+CD62L+T cells in vivo showed the existence of memory T cells (see FIG. 12).

PVP22b SA Hexapro UTR
(SEQ ID NO: 61)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATgTTTGTGTTCCTGGTGCTGCTGCCACTGGTGTCCAGCCAGTGTGTGAACTTCACCACCAG
GACCCAACTTCCTCCTGCCTACACCAACTCCTTCACCAGGGGAGTCTACTACCCTGACAAGGTGTTCA
GGTCCTCTGTGCTGCACAGCACCCAGGACCTGTTCCTGCCATTCTTCAGCAATGTGACCTGGTTCCAT
GCCATCCATGTGTCTGGCACCAATGGCACCAAGAGGTTTGCCAACCCTGTGCTGCCATTCAATGATGG
AGTCTACTTTGCCAGCACAGAGAAGAGCAACATCATCAGGGGCTGGATTTTTGGCACCACCCTGGACA
GCAAGACCCAGTCCCTGCTGATTGTGAACAATGCCACCAATGTGGTGATTAAGGTGTGTGAGTTCCAG
TTCTGTAATGACCCATTCCTGGGAGTCTACTACCACAAGAACAACAAGTCCTGGATGGAGTCTGAGTT
CAGGGTCTACTCCTCTGCCAACAACTGTACCTTTGAATATGTGAGCCAACCATTCCTGATGGACTTGG
AGGGCAAGCAGGGCAACTTCAAGAACCTGAGGGAGTTTGTGTTCAAGAACATTGATGGCTACTTCAAG
ATTTACAGCAAACACACACCAATCAACCTGGTGAGGGGCCTGCCACAGGGCTTCTCTGCCTTGGAACC
ACTGGTGGACCTGCCAATTGGCATCAACATCACCAGGTTCCAGACCCTGCTGGCTCTGCACATTTCCT
ACCTGACACCTGGAGACTCCTCCTCTGGCTGGACAGCAGGAGCAGCAGCCTACTATGTGGGCTACCTC
CAACCAAGGACCTTCCTGCTGAAATACAATGAGAATGGCACCATCACAGATGCTGTGGACTGTGCCCT
GGACCCACTGTCTGAGACCAAGTGTACCCTGAAATCCTTCACAGTGGAGAAGGGCATCTACCAGACCA
GCAACTTCAGGGTCCAACCAACAGAGAGCATTGTGAGGTTTCCAAACATCACCAACCTGTGTCCATTT
GGAGAGGTGTTCAATGCCACCAGGTTTGCCTCTGTCTATGCCTGGAACAGGAAGAGGATTAGCAACTG
TGTGGCTGACTACTCTGTGCTCTACAACTCTGCCTCCTTCAGCACCTTCAAGTGTTATGGAGTGAGCC
CAACCAAACTGAATGACCTGTGTTTCACCAATGTCTATGCTGACTCCTTTGTGATTAGGGGAGATGAG
GTGAGACAGATTGCCCCTGGACAAACAGGCAACATTGCTGACTACAACTACAAACTGCCTGATGACTT
CACAGGCTGTGTGATTGCCTGGAACAGCAACAACCTGGACAGCAAGGTGGGAGGCAACTACAACTACC
TCTACAGACTGTTCAGGAAGAGCAACCTGAAACCATTTGAGAGGGACATCAGCACAGAGATTTACCAG
GCTGGCAGCACACCATGTAATGGAGTGAAGGGCTTCAACTGTTACTTTCCACTCCAATCCTATGGCTT
CCAACCAACCTACGGAGTGGGCTACCAACCATACAGGGTGGTGGTGCTGTCCTTTGAACTGCTCCATG
CCCCTGCCACAGTGTGTGGACCAAAGAAGAGCACCAACCTGGTGAAGAACAAGTGTGTGAACTTCAAC
TTCAATGGACTGACAGGCACAGGAGTGCTGACAGAGAGCAACAAGAAGTTCCTGCCATTCCAACAGTT
TGGCAGGGACATTGCTGACACCACAGATGCTGTGAGGGACCCACAGACCTTGGAGATTCTGGACATCA
CACCATGTTCCTTTGGAGGAGTGTCTGTGATTACACCTGGCACCAACACCAGCAACCAGGTGGCTGTG
CTCTACCAGGATGTGAACTGTACTGAGGTGCCTGTGGCTATCCATGCTGACCAACTTACACCAACCTG
GAGGGTCTACAGCACAGGCAGCAATGTGTTCCAGACCAGGGCTGGCTGTCTGATTGGAGCAGAGCATG
TGAACAACTCCTATGAGTGTGACATCCCAATTGGAGCAGGCATCTGTGCCTCCTACCAGACCCAGACC
AACAGCCCAGGCTCTGCATCTTCTGTGGCAAGCCAGAGCATCATTGCCTACACAATGAGTCTGGGAGT
GGAGAACTCTGTGGCTTACAGCAACAACAGCATTGCCATCCCAACCAACTTCACCATCTCTGTGACCA
CAGAGATTCTGCCTGTGAGTATGACCAAGACCTCTGTGGACTGTACAATGTATATCTGTGGAGACAGC
ACAGAGTGTAGCAACCTGCTGCTCCAATATGGCTCCTTCTGTACCCAACTTAACAGGGCTCTGACAGG
CATTGCTGTGGAACAGGACAAGAACACCCAGGAGGTGTTTGCCCAGGTGAAGCAGATTTACAAGACAC
CTCCAATCAAGGACTTTGGAGGCTTCAACTTCAGCCAGATTCTGCCTGACCCAAGCAAGCCAAGCAAG
AGGTCCCCTATTGAGGACCTGCTGTTCAACAAGGTGACCCTGGCTGATGCTGGCTTCATCAAGCAATA
TGGAGACTGTCTGGGAGACATCGCTGCTAGGGACCTGATTTGTGCCCAGAAGTTCAATGGACTGACAG
TGCTGCCTCCACTGCTGACAGATGAGATGATTGCCCAATACACCTCTGCCCTGCTGGCTGGAACCATC
ACCTCTGGATGGACCTTTGGAGCAGGACCAGCCCTCCAAATCCCATTTCCAATGCAGATGGCTTACAG
GTTCAATGGCATTGGAGTGACCCAGAATGTGCTCTATGAGAACCAGAAACTGATTGCCAACCAGTTCA
ACTCTGCCATTGGCAAGATTCAGGACTCCCTGTCCAGCACACCATCTGCCCTGGGCAAGCTCCAAGAT
GTGGTGAACCAGAATGCCCAGGCTCTGAACACCCTGGTGAAGCAACTTTCCAGCAACTTTGGAGCCAT
CTCCTCTGTGCTGAATGACATCCTGAGCAGACTGGACCCACCAGAGGCTGAGGTCCAGATTGACAGAC
TGATTACAGGCAGACTCCAATCCCTCCAAACCTATGTGACCCAACAACTTATCAGGGCTGCTGAGATT
AGGGCATCTGCCAACCTGGCTGCCACCAAGATGAGTGAGTGTGTGCTGGGACAAAGCAAGAGGGTGGA
CTTCTGTGGCAAGGGCTACCACCTGATGAGTTTTCCACAGTCTGCCCCTCATGGAGTGGTGTTCCTGC
ATGTGACCTATGTGCCTGCCCAGGAGAAGAACTTCACCACAGCCCCTGCCATCTGCCATGATGGCAAG
GCTCACTTTCCAAGGGAGGGAGTGTTTGTGAGCAATGGCACCCACTGGTTTGTGACCCAGAGGAACTT
CTATGAACCACAGATTATCACCACAGACAACACCTTTGTGTCTGGCAACTGTGATGTGGTGATTGGCA
TTGTGAACAACACAGTCTATGACCCACTCCAACCTGAACTGGACTCCTTCAAGGAGGAACTGGACAAA
TACTTCAAGAACCACACCAGCCCTGATGTGGACCTGGGAGACATCTCTGGCATCAATGCCTCTGTGGT
GAACATCCAGAAGGAGATTGACAGACTGAATGAGGTGGCTAAGAACCTGAATGAGTCCCTGATTGACC
TCCAAGAACTGGGCAAATATGAACAATACATCAAGTGGCCATGGTACATCTGGCTGGGCTTCATTGCT
GGACTGATTGCCATTGTGATGGTGACCATAATGCTGTGTTGTATGACCTCCTGTTGTTCCTGTCTGAA
AGGCTGTTGTTCCTGTGGCTCCTGTTGTAAGTTTGATGAGGATGACTCTGAACCTGTGCTGAAAGGAG
TGAAACTGCACTACACCTGATGACTCGAGCTGGTACTGCATGCACGCAATGCTAGCTGCCCCTTTCCC
GTCCTGGGTACCCCGAGTCTCCCCCGACCTCGGGTCCCAGGTATGCTCCCACCTCCACCTGCCCCACT
CACCACCTCTGCTAGTTCCAGACACCTCCCAAGCACGCAGCAATGCAGCTCAAAACGCTTAGCCTAGC
CACACCCCCACGGGAAACAGCAGTGATTAACCTTTAGCAATAAACGAAAGTTTAACTAAGCTATACTA
ACCCCAGGGTTGGTCAATTTCGTGCCAGCCACACCCTGGAGCTAGCaaaccgctgatcagcctcgact
gtgccttctagttgccagccatctgttgtttgcccctcccccgtgccttccttgaccctggaaggtgc
cactcccactgtcctttcctaataaaatgaggaaattgcatcgcattgtctgagtaggtgtcattcta
ttctggggggtggggtggggcaggacagcaagggggaggattgggaagacaatagcaggcatgctggg
gatgcggtgggctctatggcttctgaggcggaaagaaccagctggggctctagggggtatccccacgc
gccctgtagcggcgcattaagcgcggcgggtgtggtggttacgcgcagcgtgaccgctacacttgcca
gcgccctagcgcccgctcctttcgctttcttcccttcctttctcgccacgttcgccggctttccccgt
caagctctaaatcgggggctccctttagggttccgatttagtgctttacggcacctcgaccccaaaaa
acttgattagggtgatggttcacgtagtgggccatcgccctgatagacggtttttcgccctttgacgt
tggagtccacgttctttaatagtggactcttgttccaaactggaacaacactcaaccctatctcggtc
tattcttttgatttataagggattttgccgatttcggcctattggttaaaaaatgagctgatttaaca
aaaatttaacgcgaattaattctgtggaatgtgtgtcagttagggtgtggaaagtccccaggctcccc
agcaggcagaagtatgcaaagcatgcatctcaattagtcagcaaccaggtgtggaaagtccccaggct
ccccagcaggcagaagtatgcaaagcatgcatctcaattagtcagcaaccatagtcccgcccctaact
ccgcccatcccgcccctaactccgcccagttccgcccattctccgccccatggctgactaattttttt
tatttatgcagaggccgaggccgcctctgcctctgagctattccagaagtagtgaggaggcttttttg
gaggcctaggcttttgcaaaaagctcccgggagcttgtatatccattttcggatctgatcaagagaca
ggatgaggatcgtttcgcatgattgaacaagatggattgcacgcaggttctccggccgcttgggtgga
gaggctattcggctatgactgggcacaacagacaatcggctgctctgatgccgccgtgttccggctgt
cagcgcaggggcgcccggttctttttgtcaagaccgacctgtccggtgccctgaatgaactgcaggac
gaggcagcgcggctatcgtggctggccacgacgggcgttccttgcgcagctgtgctcgacgttgtcac
tgaagcgggaagggactggctgctattgggcgaagtgccggggcaggatctcctgtcatctcaccttg
ctcctgccgagaaagtatccatcatggctgatgcaatgcggcggctgcatacgcttgatccggctacc
tgcccattcgaccaccaagcgaaacatcgcatcgagcgagcacgtactcggatggaagccggtcttgt
cgatcaggatgatctggacgaagagcatcaggggctcgcgccagccgaactgttcgccaggctcaagg
cgcgcatgcccgacggcgaggatctcgtcgtgacccatggcgatgcctgcttgccgaatatcatggtg
gaaaatggccgcttttctggattcatcgactgtggccggctgggtgtggcggaccgctatcaggacat
agcgttggctacccgtgatattgctgaagagcttggcggcgaatgggctgaccgcttcctcgtgcttt
acggtatcgccgctcccgattcgcagcgcatcgccttctatcgccttcttgacgagttcttctgagcg
ggactctggggttcgaaatgaccgaccaagcgacgcccaacctgccatcacgagatttcgattccacc
gccgccttctatgaaaggttgggcttcggaatcgttttccgggacgccggctggatgatcctccagcg
cggggatctcatgctggagttcttcgcccaccccaacttgtttattgcagcttataatggttacaaat
aaagcaatagcatcacaaatttcacaaataaagcatttttttcactgcattctagttgtggtttgtcc
aaactcatcaatgtatcttatcatgtctgtataccgtcgacctctagctagagcttggcgtaatcatg
gtcatagctgtttcctgtgtgaaattgttatccgctcacaattccacacaacatacgagccggaagca
taaagtgtaaagcctggggtgcctaatgagtgagctaactcacattaattgcgttgcgctcactgccc
gctttccagtcgggaaacctgtcgtgccagctgcattaatgaatcggccaacgcgcggggagaggcgg
tttgcgtattgggcgctcttccgcttcctcgctcactgactcgctgcgctcggtcgttcggctgcggc
gagcggtatcagctcactcaaaggcggtaatacggttatccacagaatcaggggataacgcaggaaag
aacatgtgagcaaaaggccagcaaaaggccaggaaccgtaaaaaggccgcgttgctggcgtttttcca
taggctccgcccccctgacgagcatcacaaaaatcgacgctcaagtcagaggtggcgaaacccgacag
gactataaagataccaggcgtttccccctggaagctccctcgtgcgctctcctgttccgaccctgccg
cttaccggatacctgtccgcctttctcccttcgggaagcgtggcgctttctcatagctcacgctgtag
gtatctcagttcggtgtaggtcgttcgctccaagctgggctgtgtgcacgaaccccccgttcagcccg
accgctgcgccttatccggtaactatcgtcttgagtccaacccggtaagacacgacttatcgccactg
gcagcagccactggtaacaggattagcagagcgaggtatgtaggcggtgctacagagttcttgaagtg
gtggcctaactacggctacactagaagaacagtatttggtatctgcgctctgctgaagccagttacct
tcggaaaaagagttggtagctcttgatccggcaaacaaaccaccgctggtagcggtggtttttttgtt
tgcaagcagcagattacgcgcagaaaaaaaggatctcaagaagatcctttgatcttttctacggggtc
tgacgctcagtggaacgaaaactcacgttaagggattttggtcatgagattatcaaaaaggatcttca
cctagatccttttaaattaaaaatgaagttttaaatcaatctaaagtatatatgagtaaacttggtct
gacagttaccaatgcttaatcagtgaggcacctatctcagcgatctgtctatttcgttcatccatagt
tgcctgactccccgtcgtgtagataactacgatacgggagggcttaccatctggccccagtgctgcaa
tgataccgcgagacccacgctcaccggctccagatttatcagcaataaaccagccagccggaagggcc
gagcgcagaagtggtcctgcaactttatccgcctccatccagtctattaattgttgccgggaagctag
agtaagtagttcgccagttaatagtttgcgcaacgttgttgccattgctacaggcatcgtggtgtcac
gctcgtcgtttggtatggcttcattcagctccggttcccaacgatcaaggcgagttacatgatccccc
atgttgtgcaaaaaagcggttagctccttcggtcctccgatcgttgtcagaagtaagttggccgcagt
gttatcactcatggttatggcagcactgcataattctcttactgtcatgccatccgtaagatgctttt
ctgtgactggtgagtactcaaccaagtcattctgagaatagtgtatgcggcgaccgagttgctcttgc
ccggcgtcaatacgggataataccgcgccacatagcagaactttaaaagtgctcatcattggaaaacg
ttcttcggggcgaaaactctcaaggatcttaccgctgttgagatccagttcgatgtaacccactcgtg
cacccaactgatcttcagcatcttttactttcaccagcgtttctgggtgagcaaaaacaggaaggcaa
aatgccgcaaaaaagggaataagggcgacacggaaatgttgaatactcatactcttcctttttcaata
ttattgaagcatttatcagggttattgtctcatgagcggatacatatttgaatgtatttagaaaaata
aacaaataggggttccgcgcacatttccccgaaaagtgccacctgacgtc
PVP28 pcDNA3.1 Indian B.1.617
(SEQ ID NO: 62)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccATGTTTGTGTTCCTGGTGCTGCTGCCACTGGTGTCCAGCCAGTGTGTG
AACCTGACCACCAGGACCCAACTTCCTCCTGCCTACACCAACTCCTTCACCAGGGGAGTCTACTACCC
TGACAAGGTGTTCAGGTCCTCTGTGCTGCACAGCACCCAGGACCTGTTCCTGCCATTCTTCAGCAATG
TGACCTGGTTCCATGCCATCCATGTGTCTGGCACCAATGGCACCAAGAGGTTTGACAACCCTGTGCTG
CCATTCAATGATGGAGTCTACTTTGCCAGCACAGAGAAGAGCAACATCATCAGGGGCTGGATTTTTGG
CACCACCCTGGACAGCAAGACCCAGTCCCTGCTGATTGTGAACAATGCCACCAATGTGGTGATTAAGG
TGTGTGAGTTCCAGTTCTGTAATGACCCATTCCTGGACGTCTACTACCACAAGAACAACAAGTCCTGG
ATGAAGTCTGAGTTCAGGGTCTACTCCTCTGCCAACAACTGTACCTTTGAATATGTGAGCCAACCATT
CCTGATGGACTTGGAGGGCAAGCAGGGCAACTTCAAGAACCTGAGGGAGTTTGTGTTCAAGAACATTG
ATGGCTACTTCAAGATTTACAGCAAACACACACCAATCAACCTGGTGAGGGACCTGCCACAGGGCTTC
TCTGCCTTGGAACCACTGGTGGACCTGCCAATTGGCATCAACATCACCAGGTTCCAGACCCTGCTGGC
TCTGCACAGGTCCTACCTGACACCTGGAGACTCCTCCTCTGGCTGGACAGCAGGAGCAGCAGCCTACT
ATGTGGGCTACCTCCAACCAAGGACCTTCCTGCTGAAATACAATGAGAATGGCACCATCACAGATGCT
GTGGACTGTGCCCTGGACCCACTGTCTGAGACCAAGTGTACCCTGAAATCCTTCACAGTGGAGAAGGG
CATCTACCAGACCAGCAACTTCAGGGTCCAACCAACAGAGAGCATTGTGAGGTTTCCAAACATCACCA
ACCTGTGTCCATTTGGAGAGGTGTTCAATGCCACCAGGTTTGCCTCTGTCTATGCCTGGAACAGGAAG
AGGATTAGCAACTGTGTGGCTGACTACTCTGTGCTCTACAACTCTGCCTCCTTCAGCACCTTCAAGTG
TTATGGAGTGAGCCCAACCAAACTGAATGACCTGTGTTTCACCAATGTCTATGCTGACTCCTTTGTGA
TTAGGGGAGATGAGGTGAGACAGATTGCCCCTGGACAAACAGGCAAGATTGCTGACTACAACTACAAA
CTGCCTGATGACTTCACAGGCTGTGTGATTGCCTGGAACAGCAACAACCTGGACAGCAAGGTGGGAGG
CAACTACAACTACAGGTACAGACTGTTCAGGAAGAGCAACCTGAAACCATTTGAGAGGGACATCAGCA
CAGAGATTTACCAGGCTGGCAGCACACCATGTAATGGAGTGCAGGGCTTCAACTGTTACTTTCCACTC
CAATCCTATGGCTTCCAACCAACCAATGGAGTGGGCTACCAACCATACAGGGTGGTGGTGCTGTCCTT
TGAACTGCTCCATGCCCCTGCCACAGTGTGTGGACCAAAGAAGAGCACCAACCTGGTGAAGAACAAGT
GTGTGAACTTCAACTTCAATGGACTGACAGGCACAGGAGTGCTGACAGAGAGCAACAAGAAGTTCCTG
CCATTCCAACAGTTTGGCAGGGACATTGCTGACACCACAGATGCTGTGAGGGACCCACAGACCTTGGA
GATTCTGGACATCACACCATGTTCCTTTGGAGGAGTGTCTGTGATTACACCTGGCACCAACACCAGCA
ACCAGGTGGCTGTGCTCTACCAGGGAGTGAACTGTACTGAGGTGCCTGTGGCTATCCATGCTGACCAA
CTTACACCAACCTGGAGGGTCTACAGCACAGGCAGCAATGTGTTCCAGACCAGGGCTGGCTGTCTGAT
TGGAGCAGAGCATGTGAACAACTCCTATGAGTGTGACATCCCAATTGGAGCAGGCATCTGTGCCTCCT
ACCAGACCCAGACCAACAGCAGGAGGAGGGCAAGGTCTGTGGCAAGCCAGAGCATCATTGCCTACACA
ATGAGTCTGGGAGCAGAGAACTCTGTGGCTTACAGCAACAACAGCATTGCCATCCCAACCAACTTCAC
CATCTCTGTGACCACAGAGATTCTGCCTGTGAGTATGACCAAGACCTCTGTGGACTGTACAATGTATA
TCTGTGGAGACAGCACAGAGTGTAGCAACCTGCTGCTCCAATATGGCTCCTTCTGTACCCAACTTAAC
AGGGCTCTGACAGGCATTGCTGTGGAACAGGACAAGAACACCCAGGAGGTGTTTGCCCAGGTGAAGCA
GATTTACAAGACACCTCCAATCAAGGACTTTGGAGGCTTCAACTTCAGCCAGATTCTGCCTGACCCAA
GCAAGCCAAGCAAGAGGTCCTTCATTGAGGACCTGCTGTTCAACAAGGTGACCCTGGCTGATGCTGGC
TTCATCAAGCAATATGGAGACTGTCTGGGAGACATTGCTGCCAGGGACCTGATTTGTGCCCAGAAGTT
CAATGGACTGACAGTGCTGCCTCCACTGCTGACAGATGAGATGATTGCCCAATACACCTCTGCCCTGC
TGGCTGGCACCATCACCTCTGGCTGGACCTTTGGAGCAGGAGCAGCCCTCCAAATCCCATTTGCTATG
CAGATGGCTTACAGGTTCAATGGCATTGGAGTGACCCAGAATGTGCTCTATGAGAACCAGAAACTGAT
TGCCAACCAGTTCAACTCTGCCATTGGCAAGATTCAGGACTCCCTGTCCAGCACAGCCTCTGCCCTGG
GCAAACTCCAAGATGTGGTGAACCAGAATGCCCAGGCTCTGAACACCCTGGTGAAGCAACTTTCCAGC
AACTTTGGAGCCATCTCCTCTGTGCTGAATGACATCCTGAGCAGACTGGACAAGGTGGAGGCTGAGGT
CCAGATTGACAGACTGATTACAGGCAGACTCCAATCCCTCCAAACCTATGTGACCCAACAACTTATCA
GGGCTGCTGAGATTAGGGCATCTGCCAACCTGGCTGCCACCAAGATGAGTGAGTGTGTGCTGGGACAA
AGCAAGAGGGTGGACTTCTGTGGCAAGGGCTACCACCTGATGAGTTTTCCACAGTCTGCCCCTCATGG
AGTGGTGTTCCTGCATGTGACCTATGTGCCTGCCCATGAGAAGAACTTCACCACAGCCCCTGCCATCT
GCCATGATGGCAAGGCTCACTTTCCAAGGGAGGGAGTGTTTGTGAGCAATGGCACCCACTGGTTTGTG
ACCCAGAGGAACTTCTATGAACCACAGATTATCACCACAGACAACACCTTTGTGTCTGGCAACTGTGA
TGTGGTGATTGGCATTGTGAACAACACAGTCTATGACCCACTCCAACCTGAACTGGACTCCTTCAAGG
AGGAACTGGACAAATACTTCAAGAACCACACCAGCCCTGATGTGGACCTGGGAGACATCTCTGGCATC
AATGCCTCTGTGGTGAACATCCAGAAGGAGATTGACAGACTGAATGAGGTGGCTAAGAACCTGAATGA
GTCCCTGATTGACCTCCAAGAACTGGGCAAATATGAACAATACATCAAGTGGCCATGGTACATCTGGC
TGGGCTTCATTGCTGGACTGATTGCCATTGTGATGGTGACCATAATGCTGTGTTGTATGACCTCCTGT
TGTTCCTGTCTGAAAGGCTGTTGTTCCTGTGGCTCCTGTTGTAAGTTTGATGAGGATGACTCTGAACC
TGTGCTGAAAGGAGTGAAACTGCACTACACCTGAggatccgagctcggtaccaagcttaagtttaaac
cgctgatcagcctcgactgtgccttctagttgccagccatctgttgtttgcccctcccccgtgccttc
cttgaccctggaaggtgccactcccactgtcctttcctaataaaatgaggaaattgcatcgcattgtc
tgagtaggtgtcattctattctggggggtggggggggcaggacagcaagggggaggattgggaagac
aatagcaggcatgctggggatgcggtgggctctatggcttctgaggcggaaagaaccagctggggctc
tagggggtatccccacgcgccctgtagcggcgcattaagcgcggcgggtgtggtggttacgcgcagcg
tgaccgctacacttgccagcgccctagcgcccgctcctttcgctttcttcccttcctttctcgccacg
ttcgccggctttccccgtcaagctctaaatcgggggctccctttagggttccgatttagtgctttacg
gcacctcgaccccaaaaaacttgattagggtgatggttcacgtagtgggccatcgccctgatagacgg
tttttcgccctttgacgttggagtccacgttctttaatagtggactcttgttccaaactggaacaaca
ctcaaccctatctcggtctattcttttgatttataagggattttgccgatttcggcctattggttaaa
aaatgagctgatttaacaaaaatttaacgcgaattaattctgtggaatgtgtgtcagttagggtgtgg
aaagtccccaggctccccagcaggcagaagtatgcaaagcatgcatctcaattagtcagcaaccaggt
gtggaaagtccccaggctccccagcaggcagaagtatgcaaagcatgcatctcaattagtcagcaacc
atagtcccgcccctaactccgcccatcccgcccctaactccgcccagttccgcccattctccgcccca
tggctgactaattttttttatttatgcagaggccgaggccgcctctgcctctgagctattccagaagt
agtgaggaggcttttttggaggcctaggcttttgcaaaaagctcccgggagcttgtatatccattttc
ggatctgatcaagagacaggatgaggatcgtttcgcatgattgaacaagatggattgcacgcaggttc
tccggccgcttgggtggagaggctattcggctatgactgggcacaacagacaatcggctgctctgatg
ccgccgtgttccggctgtcagcgcaggggcgcccggttctttttgtcaagaccgacctgtccggtgcc
ctgaatgaactgcaggacgaggcagcgcggctatcgtggctggccacgacgggcgttccttgcgcagc
tgtgctcgacgttgtcactgaagcgggaagggactggctgctattgggcgaagtgccggggcaggatc
tcctgtcatctcaccttgctcctgccgagaaagtatccatcatggctgatgcaatgcggcggctgcat
acgcttgatccggctacctgcccattcgaccaccaagcgaaacatcgcatcgagcgagcacgtactcg
gatggaagccggtcttgtcgatcaggatgatctggacgaagagcatcaggggctcgcgccagccgaac
tgttcgccaggctcaaggcgcgcatgcccgacggcgaggatctcgtcgtgacccatggcgatgcctgc
ttgccgaatatcatggtggaaaatggccgcttttctggattcatcgactgtggccggctgggtgtggc
ggaccgctatcaggacatagcgttggctacccgtgatattgctgaagagcttggcggcgaatgggctg
accgcttcctcgtgctttacggtatcgccgctcccgattcgcagcgcatcgccttctatcgccttctt
gacgagttcttctgagcgggactctggggttcgaaatgaccgaccaagcgacgcccaacctgccatca
cgagatttcgattccaccgccgccttctatgaaaggttgggcttcggaatcgttttccgggacgccgg
ctggatgatcctccagcgcggggatctcatgctggagttcttcgcccaccccaacttgtttattgcag
cttataatggttacaaataaagcaatagcatcacaaatttcacaaataaagcatttttttcactgcat
tctagttgtggtttgtccaaactcatcaatgtatcttatcatgtctgtataccgtcgacctctagcta
gagcttggcgtaatcatggtcatagctgtttcctgtgtgaaattgttatccgctcacaattccacaca
acatacgagccggaagcataaagtgtaaagcctggggtgcctaatgagtgagctaactcacattaatt
gcgttgcgctcactgcccgctttccagtcgggaaacctgtcgtgccagctgcattaatgaatcggcca
acgcgcggggagaggcggtttgcgtattgggcgctcttccgcttcctcgctcactgactcgctgcgct
cggtcgttcggctgcggcgagcggtatcagctcactcaaaggcggtaatacggttatccacagaatca
ggggataacgcaggaaagaacatgtgagcaaaaggccagcaaaaggccaggaaccgtaaaaaggccgc
gttgctggcgtttttccataggctccgcccccctgacgagcatcacaaaaatcgacgctcaagtcaga
ggtggcgaaacccgacaggactataaagataccaggcgtttccccctggaagctccctcgtgcgctct
cctgttccgaccctgccgcttaccggatacctgtccgcctttctcccttcgggaagcgtggcgctttc
tcatagctcacgctgtaggtatctcagttcggtgtaggtcgttcgctccaagctgggctgtgtgcacg
aaccccccgttcagcccgaccgctgcgccttatccggtaactatcgtcttgagtccaacccggtaaga
cacgacttatcgccactggcagcagccactggtaacaggattagcagagcgaggtatgtaggcggtgc
tacagagttcttgaagtggtggcctaactacggctacactagaagaacagtatttggtatctgcgctc
tgctgaagccagttaccttcggaaaaagagttggtagctcttgatccggcaaacaaaccaccgctggt
agcggtggtttttttgtttgcaagcagcagattacgcgcagaaaaaaaggatctcaagaagatccttt
gatcttttctacggggtctgacgctcagtggaacgaaaactcacgttaagggattttggtcatgagat
tatcaaaaaggatcttcacctagatccttttaaattaaaaatgaagttttaaatcaatctaaagtata
tatgagtaaacttggtctgacagttaccaatgcttaatcagtgaggcacctatctcagcgatctgtct
atttcgttcatccatagttgcctgactccccgtcgtgtagataactacgatacgggagggcttaccat
ctggccccagtgctgcaatgataccgcgagacccacgctcaccggctccagatttatcagcaataaac
cagccagccggaagggccgagcgcagaagtggtcctgcaactttatccgcctccatccagtctattaa
ttgttgccgggaagctagagtaagtagttcgccagttaatagtttgcgcaacgttgttgccattgcta
caggcatcgtggtgtcacgctcgtcgtttggtatggcttcattcagctccggttcccaacgatcaagg
cgagttacatgatcccccatgttgtgcaaaaaagcggttagctccttcggtcctccgatcgttgtcag
aagtaagttggccgcagtgttatcactcatggttatggcagcactgcataattctcttactgtcatgc
catccgtaagatgcttttctgtgactggtgagtactcaaccaagtcattctgagaatagtgtatgcgg
cgaccgagttgctcttgcccggcgtcaatacgggataataccgcgccacatagcagaactttaaaagt
gctcatcattggaaaacgttcttcggggcgaaaactctcaaggatcttaccgctgttgagatccagtt
cgatgtaacccactcgtgcacccaactgatcttcagcatcttttactttcaccagcgtttctgggtga
gcaaaaacaggaaggcaaaatgccgcaaaaaagggaataagggcgacacggaaatgttgaatactcat
actcttcctttttcaatattattgaagcatttatcagggttattgtctcatgagcggatacatatttg
aatgtatttagaaaaataaacaaataggggttccgcgcacatttccccgaaaagtgccacctgacgtc
PVP29b Indian B.1.617 Hexapro UTR
(SEQ ID NO: 63)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATgTTTGTGTTCCTGGTGCTGCTGCCACTGGTGTCCAGCCAGTGTGTGAACCTGACCACCAG
GACCCAACTTCCTCCTGCCTACACCAACTCCTTCACCAGGGGAGTCTACTACCCTGACAAGGTGTTCA
GGTCCTCTGTGCTGCACAGCACCCAGGACCTGTTCCTGCCATTCTTCAGCAATGTGACCTGGTTCCAT
GCCATCCATGTGTCTGGCACCAATGGCACCAAGAGGTTTGACAACCCTGTGCTGCCATTCAATGATGG
AGTCTACTTTGCCAGCACAGAGAAGAGCAACATCATCAGGGGCTGGATTTTTGGCACCACCCTGGACA
GCAAGACCCAGTCCCTGCTGATTGTGAACAATGCCACCAATGTGGTGATTAAGGTGTGTGAGTTCCAG
TTCTGTAATGACCCATTCCTGGACGTCTACTACCACAAGAACAACAAGTCCTGGATGAAGTCTGAGTT
CAGGGTCTACTCCTCTGCCAACAACTGTACCTTTGAATATGTGAGCCAACCATTCCTGATGGACTTGG
AGGGCAAGCAGGGCAACTTCAAGAACCTGAGGGAGTTTGTGTTCAAGAACATTGATGGCTACTTCAAG
ATTTACAGCAAACACACACCAATCAACCTGGTGAGGGACCTGCCACAGGGCTTCTCTGCCTTGGAACC
ACTGGTGGACCTGCCAATTGGCATCAACATCACCAGGTTCCAGACCCTGCTGGCTCTGCACAGGTCCT
ACCTGACACCTGGAGACTCCTCCTCTGGCTGGACAGCAGGAGCAGCAGCCTACTATGTGGGCTACCTC
CAACCAAGGACCTTCCTGCTGAAATACAATGAGAATGGCACCATCACAGATGCTGTGGACTGTGCCCT
GGACCCACTGTCTGAGACCAAGTGTACCCTGAAATCCTTCACAGTGGAGAAGGGCATCTACCAGACCA
GCAACTTCAGGGTCCAACCAACAGAGAGCATTGTGAGGTTTCCAAACATCACCAACCTGTGTCCATTT
GGAGAGGTGTTCAATGCCACCAGGTTTGCCTCTGTCTATGCCTGGAACAGGAAGAGGATTAGCAACTG
TGTGGCTGACTACTCTGTGCTCTACAACTCTGCCTCCTTCAGCACCTTCAAGTGTTATGGAGTGAGCC
CAACCAAACTGAATGACCTGTGTTTCACCAATGTCTATGCTGACTCCTTTGTGATTAGGGGAGATGAG
GTGAGACAGATTGCCCCTGGACAAACAGGCAAGATTGCTGACTACAACTACAAACTGCCTGATGACTT
CACAGGCTGTGTGATTGCCTGGAACAGCAACAACCTGGACAGCAAGGTGGGAGGCAACTACAACTACA
GGTACAGACTGTTCAGGAAGAGCAACCTGAAACCATTTGAGAGGGACATCAGCACAGAGATTTACCAG
GCTGGCAGCACACCATGTAATGGAGTGCAGGGCTTCAACTGTTACTTTCCACTCCAATCCTATGGCTT
CCAACCAACCAATGGAGTGGGCTACCAACCATACAGGGTGGTGGTGCTGTCCTTTGAACTGCTCCATG
CCCCTGCCACAGTGTGTGGACCAAAGAAGAGCACCAACCTGGTGAAGAACAAGTGTGTGAACTTCAAC
TTCAATGGACTGACAGGCACAGGAGTGCTGACAGAGAGCAACAAGAAGTTCCTGCCATTCCAACAGTT
TGGCAGGGACATTGCTGACACCACAGATGCTGTGAGGGACCCACAGACCTTGGAGATTCTGGACATCA
CACCATGTTCCTTTGGAGGAGTGTCTGTGATTACACCTGGCACCAACACCAGCAACCAGGTGGCTGTG
CTCTACCAGGGAGTGAACTGTACTGAGGTGCCTGTGGCTATCCATGCTGACCAACTTACACCAACCTG
GAGGGTCTACAGCACAGGCAGCAATGTGTTCCAGACCAGGGCTGGCTGTCTGATTGGAGCAGAGCATG
TGAACAACTCCTATGAGTGTGACATCCCAATTGGAGCAGGCATCTGTGCCTCCTACCAGACCCAGACC
AACAGCAGGGGCTCTGCATCTTCTGTGGCAAGCCAGAGCATCATTGCCTACACAATGAGTCTGGGAGC
AGAGAACTCTGTGGCTTACAGCAACAACAGCATTGCCATCCCAACCAACTTCACCATCTCTGTGACCA
CAGAGATTCTGCCTGTGAGTATGACCAAGACCTCTGTGGACTGTACAATGTATATCTGTGGAGACAGC
ACAGAGTGTAGCAACCTGCTGCTCCAATATGGCTCCTTCTGTACCCAACTTAACAGGGCTCTGACAGG
CATTGCTGTGGAACAGGACAAGAACACACAGGAGGTGTTTGCCCAGGTGAAGCAGATTTACAAGACAC
CTCCAATCAAGGACTTTGGAGGCTTCAACTTCAGCCAGATTCTGCCTGACCCAAGCAAGCCAAGCAAG
AGGTCCCCTATTGAGGACCTGCTGTTCAACAAGGTGACCCTGGCTGATGCTGGCTTCATCAAGCAATA
TGGAGACTGTCTGGGAGACATTGCTGCCAGGGACCTGATTTGTGCCCAGAAGTTCAATGGACTGACAG
TGCTGCCTCCACTGCTGACAGATGAGATGATTGCCCAATACACCTCTGCCCTGCTGGCTGGCACCATC
ACCTCTGGCTGGACCTTTGGAGCAGGACCAGCCCTCCAAATCCCATTTCCAATGCAGATGGCTTACAG
GTTCAATGGCATTGGAGTGACCCAGAATGTGCTCTATGAGAACCAGAAACTGATTGCCAACCAGTTCA
ACTCTGCCATTGGCAAGATTCAGGACTCCCTGTCCAGCACACCATCTGCCCTGGGCAAACTCCAAGAT
GTGGTGAACCAGAATGCCCAGGCTCTGAACACCCTGGTGAAGCAACTTTCCAGCAACTTTGGAGCCAT
CTCCTCTGTGCTGAATGACATCCTGAGCAGACTGGACCCACCAGAGGCTGAGGTCCAGATTGACAGAC
TGATTACAGGCAGACTCCAATCCCTCCAAACCTATGTGACCCAACAACTTATCAGGGCTGCTGAGATT
AGGGCATCTGCCAACCTGGCTGCCACCAAGATGAGTGAGTGTGTGCTGGGACAAAGCAAGAGGGTGGA
CTTCTGTGGCAAGGGCTACCACCTGATGAGTTTTCCACAGTCTGCCCCTCATGGAGTGGTGTTCCTGC
ATGTGACCTATGTGCCTGCCCATGAGAAGAACTTCACCACAGCCCCTGCCATCTGCCATGATGGCAAG
GCTCACTTTCCAAGGGAGGGAGTGTTTGTGAGCAATGGCACCCACTGGTTTGTGACCCAGAGGAACTT
CTATGAACCACAGATTATCACCACAGACAACACCTTTGTGTCTGGCAACTGTGATGTGGTGATTGGCA
TTGTGAACAACACAGTCTATGACCCACTCCAACCTGAACTGGACTCCTTCAAGGAGGAACTGGACAAA
TACTTCAAGAACCACACCAGCCCTGATGTGGACCTGGGAGACATCTCTGGCATCAATGCCTCTGTGGT
GAACATCCAGAAGGAGATTGACAGACTGAATGAGGTGGCTAAGAACCTGAATGAGTCCCTGATTGACC
TCCAAGAACTGGGCAAATATGAACAATACATCAAGTGGCCATGGTACATCTGGCTGGGCTTCATTGCT
GGACTGATTGCCATTGTGATGGTGACCATAATGCTGTGTTGTATGACCTCCTGTTGTTCCTGTCTGAA
AGGCTGTTGTTCCTGTGGCTCCTGTTGTAAGTTTGATGAGGATGACTCTGAACCTGTGCTGAAAGGAG
TGAAACTGCACTACACCTGACTCGAGCTGGTACTGCATGCACGCAATGCTAGCTGCCCCTTTCCCGTC
CTGGGTACCCCGAGTCTCCCCCGACCTCGGGTCCCAGGTATGCTCCCACCTCCACCTGCCCCACTCAC
CACCTCTGCTAGTTCCAGACACCTCCCAAGCACGCAGCAATGCAGCTCAAAACGCTTAGCCTAGCCAC
ACCCCCACGGGAAACAGCAGTGATTAACCTTTAGCAATAAACGAAAGTTTAACTAAGCTATACTAACC
CCAGGGTTGGTCAATTTCGTGCCAGCCACACCCTGGAGCTAGCaaaccgctgatcagcctcgactgtg
ccttctagttgccagccatctgttgtttgcccctcccccgtgccttccttgaccctggaaggtgccac
tcccactgtcctttcctaataaaatgaggaaattgcatcgcattgtctgagtaggtgtcattctattc
tggggggtggggggggcaggacagcaagggggaggattgggaagacaatagcaggcatgctggggat
gcggtgggctctatggcttctgaggcggaaagaaccagctggggctctagggggtatccccacgcgcc
ctgtagcggcgcattaagcgcggcgggtgtggtggttacgcgcagcgtgaccgctacacttgccagcg
ccctagcgcccgctcctttcgctttcttcccttcctttctcgccacgttcgccggctttccccgtcaa
gctctaaatcgggggctccctttagggttccgatttagtgctttacggcacctcgaccccaaaaaact
tgattagggtgatggttcacgtagtgggccatcgccctgatagacggtttttcgccctttgacgttgg
agtccacgttctttaatagtggactcttgttccaaactggaacaacactcaaccctatctcggtctat
tcttttgatttataagggattttgccgatttcggcctattggttaaaaaatgagctgatttaacaaaa
atttaacgcgaattaattctgtggaatgtgtgtcagttagggtgtggaaagtccccaggctccccagc
aggcagaagtatgcaaagcatgcatctcaattagtcagcaaccaggtgtggaaagtccccaggctccc
cagcaggcagaagtatgcaaagcatgcatctcaattagtcagcaaccatagtcccgcccctaactccg
cccatcccgcccctaactccgcccagttccgcccattctccgccccatggctgactaattttttttat
ttatgcagaggccgaggccgcctctgcctctgagctattccagaagtagtgaggaggcttttttggag
gcctaggcttttgcaaaaagctcccgggagcttgtatatccattttcggatctgatcaagagacagga
tgaggatcgtttcgcatgattgaacaagatggattgcacgcaggttctccggccgcttgggtggagag
gctattcggctatgactgggcacaacagacaatcggctgctctgatgccgccgtgttccggctgtcag
cgcaggggcgcccggttctttttgtcaagaccgacctgtccggtgccctgaatgaactgcaggacgag
gcagcgcggctatcgtggctggccacgacgggcgttccttgcgcagctgtgctcgacgttgtcactga
agcgggaagggactggctgctattgggcgaagtgccggggcaggatctcctgtcatctcaccttgctc
ctgccgagaaagtatccatcatggctgatgcaatgcggcggctgcatacgcttgatccggctacctgc
ccattcgaccaccaagcgaaacatcgcatcgagcgagcacgtactcggatggaagccggtcttgtcga
tcaggatgatctggacgaagagcatcaggggctcgcgccagccgaactgttcgccaggctcaaggcgc
gcatgcccgacggcgaggatctcgtcgtgacccatggcgatgcctgcttgccgaatatcatggtggaa
aatggccgcttttctggattcatcgactgtggccggctgggtgtggcggaccgctatcaggacatagc
gttggctacccgtgatattgctgaagagcttggcggcgaatgggctgaccgcttcctcgtgctttacg
gtatcgccgctcccgattcgcagcgcatcgccttctatcgccttcttgacgagttcttctgagcggga
ctctggggttcgaaatgaccgaccaagcgacgcccaacctgccatcacgagatttcgattccaccgcc
gccttctatgaaaggttgggcttcggaatcgttttccgggacgccggctggatgatcctccagcgcgg
ggatctcatgctggagttcttcgcccaccccaacttgtttattgcagcttataatggttacaaataaa
gcaatagcatcacaaatttcacaaataaagcatttttttcactgcattctagttgtggtttgtccaaa
ctcatcaatgtatcttatcatgtctgtataccgtcgacctctagctagagcttggcgtaatcatggtc
atagctgtttcctgtgtgaaattgttatccgctcacaattccacacaacatacgagccggaagcataa
agtgtaaagcctggggtgcctaatgagtgagctaactcacattaattgcgttgcgctcactgcccgct
ttccagtcgggaaacctgtcgtgccagctgcattaatgaatcggccaacgcgcggggagaggcggttt
gcgtattgggcgctcttccgcttcctcgctcactgactcgctgcgctcggtcgttcggctgcggcgag
cggtatcagctcactcaaaggcggtaatacggttatccacagaatcaggggataacgcaggaaagaac
atgtgagcaaaaggccagcaaaaggccaggaaccgtaaaaaggccgcgttgctggcgtttttccatag
gctccgcccccctgacgagcatcacaaaaatcgacgctcaagtcagaggtggcgaaacccgacaggac
tataaagataccaggcgtttccccctggaagctccctcgtgcgctctcctgttccgaccctgccgctt
accggatacctgtccgcctttctcccttcgggaagcgtggcgctttctcatagctcacgctgtaggta
tctcagttcggtgtaggtcgttcgctccaagctgggctgtgtgcacgaaccccccgttcagcccgacc
gctgcgccttatccggtaactatcgtcttgagtccaacccggtaagacacgacttatcgccactggca
gcagccactggtaacaggattagcagagcgaggtatgtaggcggtgctacagagttcttgaagtggtg
gcctaactacggctacactagaagaacagtatttggtatctgcgctctgctgaagccagttaccttcg
gaaaaagagttggtagctcttgatccggcaaacaaaccaccgctggtagcggtggtttttttgtttgc
aagcagcagattacgcgcagaaaaaaaggatctcaagaagatcctttgatcttttctacggggtctga
cgctcagtggaacgaaaactcacgttaagggattttggtcatgagattatcaaaaaggatcttcacct
agatccttttaaattaaaaatgaagttttaaatcaatctaaagtatatatgagtaaacttggtctgac
agttaccaatgcttaatcagtgaggcacctatctcagcgatctgtctatttcgttcatccatagttgc
ctgactccccgtcgtgtagataactacgatacgggagggcttaccatctggccccagtgctgcaatga
taccgcgagacccacgctcaccggctccagatttatcagcaataaaccagccagccggaagggccgag
cgcagaagtggtcctgcaactttatccgcctccatccagtctattaattgttgccgggaagctagagt
aagtagttcgccagttaatagtttgcgcaacgttgttgccattgctacaggcatcgtggtgtcacgct
cgtcgtttggtatggcttcattcagctccggttcccaacgatcaaggcgagttacatgatcccccatg
ttgtgcaaaaaagcggttagctccttcggtcctccgatcgttgtcagaagtaagttggccgcagtgtt
atcactcatggttatggcagcactgcataattctcttactgtcatgccatccgtaagatgcttttctg
tgactggtgagtactcaaccaagtcattctgagaatagtgtatgcggcgaccgagttgctcttgcccg
gcgtcaatacgggataataccgcgccacatagcagaactttaaaagtgctcatcattggaaaacgttc
ttcggggcgaaaactctcaaggatcttaccgctgttgagatccagttcgatgtaacccactcgtgcac
ccaactgatcttcagcatcttttactttcaccagcgtttctgggtgagcaaaaacaggaaggcaaaat
gccgcaaaaaagggaataagggcgacacggaaatgttgaatactcatactcttcctttttcaatatta
ttgaagcatttatcagggttattgtctcatgagcggatacatatttgaatgtatttagaaaaataaac
aaataggggttccgcgcacatttccccgaaaagtgccacctgacgtc
PVP30 pcDNA3.1 Indian B.1.617 Del-19
(SEQ ID NO: 64)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccATGTTTGTGTTCCTGGTGCTGCTGCCACTGGTGTCCAGCCAGTGTGTG
AACCTGACCACCAGGACCCAACTTCCTCCTGCCTACACCAACTCCTTCACCAGGGGAGTCTACTACCC
TGACAAGGTGTTCAGGTCCTCTGTGCTGCACAGCACCCAGGACCTGTTCCTGCCATTCTTCAGCAATG
TGACCTGGTTCCATGCCATCCATGTGTCTGGCACCAATGGCACCAAGAGGTTTGACAACCCTGTGCTG
CCATTCAATGATGGAGTCTACTTTGCCAGCACAGAGAAGAGCAACATCATCAGGGGCTGGATTTTTGG
CACCACCCTGGACAGCAAGACCCAGTCCCTGCTGATTGTGAACAATGCCACCAATGTGGTGATTAAGG
TGTGTGAGTTCCAGTTCTGTAATGACCCATTCCTGGACGTCTACTACCACAAGAACAACAAGTCCTGG
ATGAAGTCTGAGTTCAGGGTCTACTCCTCTGCCAACAACTGTACCTTTGAATATGTGAGCCAACCATT
CCTGATGGACTTGGAGGGCAAGCAGGGCAACTTCAAGAACCTGAGGGAGTTTGTGTTCAAGAACATTG
ATGGCTACTTCAAGATTTACAGCAAACACACACCAATCAACCTGGTGAGGGACCTGCCACAGGGCTTC
TCTGCCTTGGAACCACTGGTGGACCTGCCAATTGGCATCAACATCACCAGGTTCCAGACCCTGCTGGC
TCTGCACAGGTCCTACCTGACACCTGGAGACTCCTCCTCTGGCTGGACAGCAGGAGCAGCAGCCTACT
ATGTGGGCTACCTCCAACCAAGGACCTTCCTGCTGAAATACAATGAGAATGGCACCATCACAGATGCT
GTGGACTGTGCCCTGGACCCACTGTCTGAGACCAAGTGTACCCTGAAATCCTTCACAGTGGAGAAGGG
CATCTACCAGACCAGCAACTTCAGGGTCCAACCAACAGAGAGCATTGTGAGGTTTCCAAACATCACCA
ACCTGTGTCCATTTGGAGAGGTGTTCAATGCCACCAGGTTTGCCTCTGTCTATGCCTGGAACAGGAAG
AGGATTAGCAACTGTGTGGCTGACTACTCTGTGCTCTACAACTCTGCCTCCTTCAGCACCTTCAAGTG
TTATGGAGTGAGCCCAACCAAACTGAATGACCTGTGTTTCACCAATGTCTATGCTGACTCCTTTGTGA
TTAGGGGAGATGAGGTGAGACAGATTGCCCCTGGACAAACAGGCAAGATTGCTGACTACAACTACAAA
CTGCCTGATGACTTCACAGGCTGTGTGATTGCCTGGAACAGCAACAACCTGGACAGCAAGGTGGGAGG
CAACTACAACTACAGGTACAGACTGTTCAGGAAGAGCAACCTGAAACCATTTGAGAGGGACATCAGCA
CAGAGATTTACCAGGCTGGCAGCACACCATGTAATGGAGTGCAGGGCTTCAACTGTTACTTTCCACTC
CAATCCTATGGCTTCCAACCAACCAATGGAGTGGGCTACCAACCATACAGGGTGGTGGTGCTGTCCTT
TGAACTGCTCCATGCCCCTGCCACAGTGTGTGGACCAAAGAAGAGCACCAACCTGGTGAAGAACAAGT
GTGTGAACTTCAACTTCAATGGACTGACAGGCACAGGAGTGCTGACAGAGAGCAACAAGAAGTTCCTG
CCATTCCAACAGTTTGGCAGGGACATTGCTGACACCACAGATGCTGTGAGGGACCCACAGACCTTGGA
GATTCTGGACATCACACCATGTTCCTTTGGAGGAGTGTCTGTGATTACACCTGGCACCAACACCAGCA
ACCAGGTGGCTGTGCTCTACCAGGGAGTGAACTGTACTGAGGTGCCTGTGGCTATCCATGCTGACCAA
CTTACACCAACCTGGAGGGTCTACAGCACAGGCAGCAATGTGTTCCAGACCAGGGCTGGCTGTCTGAT
TGGAGCAGAGCATGTGAACAACTCCTATGAGTGTGACATCCCAATTGGAGCAGGCATCTGTGCCTCCT
ACCAGACCCAGACCAACAGCAGGAGGAGGGCAAGGTCTGTGGCAAGCCAGAGCATCATTGCCTACACA
ATGAGTCTGGGAGCAGAGAACTCTGTGGCTTACAGCAACAACAGCATTGCCATCCCAACCAACTTCAC
CATCTCTGTGACCACAGAGATTCTGCCTGTGAGTATGACCAAGACCTCTGTGGACTGTACAATGTATA
TCTGTGGAGACAGCACAGAGTGTAGCAACCTGCTGCTCCAATATGGCTCCTTCTGTACCCAACTTAAC
AGGGCTCTGACAGGCATTGCTGTGGAACAGGACAAGAACACCCAGGAGGTGTTTGCCCAGGTGAAGCA
GATTTACAAGACACCTCCAATCAAGGACTTTGGAGGCTTCAACTTCAGCCAGATTCTGCCTGACCCAA
GCAAGCCAAGCAAGAGGTCCTTCATTGAGGACCTGCTGTTCAACAAGGTGACCCTGGCTGATGCTGGC
TTCATCAAGCAATATGGAGACTGTCTGGGAGACATTGCTGCCAGGGACCTGATTTGTGCCCAGAAGTT
CAATGGACTGACAGTGCTGCCTCCACTGCTGACAGATGAGATGATTGCCCAATACACCTCTGCCCTGC
TGGCTGGCACCATCACCTCTGGCTGGACCTTTGGAGCAGGAGCAGCCCTCCAAATCCCATTTGCTATG
CAGATGGCTTACAGGTTCAATGGCATTGGAGTGACCCAGAATGTGCTCTATGAGAACCAGAAACTGAT
TGCCAACCAGTTCAACTCTGCCATTGGCAAGATTCAGGACTCCCTGTCCAGCACAGCCTCTGCCCTGG
GCAAACTCCAAGATGTGGTGAACCAGAATGCCCAGGCTCTGAACACCCTGGTGAAGCAACTTTCCAGC
AACTTTGGAGCCATCTCCTCTGTGCTGAATGACATCCTGAGCAGACTGGACAAGGTGGAGGCTGAGGT
CCAGATTGACAGACTGATTACAGGCAGACTCCAATCCCTCCAAACCTATGTGACCCAACAACTTATCA
GGGCTGCTGAGATTAGGGCATCTGCCAACCTGGCTGCCACCAAGATGAGTGAGTGTGTGCTGGGACAA
AGCAAGAGGGTGGACTTCTGTGGCAAGGGCTACCACCTGATGAGTTTTCCACAGTCTGCCCCTCATGG
AGTGGTGTTCCTGCATGTGACCTATGTGCCTGCCCATGAGAAGAACTTCACCACAGCCCCTGCCATCT
GCCATGATGGCAAGGCTCACTTTCCAAGGGAGGGAGTGTTTGTGAGCAATGGCACCCACTGGTTTGTG
ACCCAGAGGAACTTCTATGAACCACAGATTATCACCACAGACAACACCTTTGTGTCTGGCAACTGTGA
TGTGGTGATTGGCATTGTGAACAACACAGTCTATGACCCACTCCAACCTGAACTGGACTCCTTCAAGG
AGGAACTGGACAAATACTTCAAGAACCACACCAGCCCTGATGTGGACCTGGGAGACATCTCTGGCATC
AATGCCTCTGTGGTGAACATCCAGAAGGAGATTGACAGACTGAATGAGGTGGCTAAGAACCTGAATGA
GTCCCTGATTGACCTCCAAGAACTGGGCAAATATGAACAATACATCAAGTGGCCATGGTACATCTGGC
TGGGCTTCATTGCTGGACTGATTGCCATTGTGATGGTGACCATAATGCTGTGTTGTATGACCTCCTGT
TGTTCCTGTCTGAAAGGCTGTTGTTCCTGTGGCTCCTGTTGTTGAggatccgagctcggtaccaagct
taagtttaaaccgctgatcagcctcgactgtgccttctagttgccagccatctgttgtttgcccctcc
cccgtgccttccttgaccctggaaggtgccactcccactgtcctttcctaataaaatgaggaaattgc
atcgcattgtctgagtaggtgtcattctattctggggggtggggggggcaggacagcaagggggagg
attgggaagacaatagcaggcatgctggggatgcggtgggctctatggcttctgaggcggaaagaacc
agctggggctctagggggtatccccacgcgccctgtagcggcgcattaagcgcggcgggtgtggtggt
tacgcgcagcgtgaccgctacacttgccagcgccctagcgcccgctcctttcgctttcttcccttcct
ttctcgccacgttcgccggctttccccgtcaagctctaaatcgggggctccctttagggttccgattt
agtgctttacggcacctcgaccccaaaaaacttgattagggtgatggttcacgtagtgggccatcgcc
ctgatagacggtttttcgccctttgacgttggagtccacgttctttaatagtggactcttgttccaaa
ctggaacaacactcaaccctatctcggtctattcttttgatttataagggattttgccgatttcggcc
tattggttaaaaaatgagctgatttaacaaaaatttaacgcgaattaattctgtggaatgtgtgtcag
ttagggtgtggaaagtccccaggctccccagcaggcagaagtatgcaaagcatgcatctcaattagtc
agcaaccaggtgtggaaagtccccaggctccccagcaggcagaagtatgcaaagcatgcatctcaatt
agtcagcaaccatagtcccgcccctaactccgcccatcccgcccctaactccgcccagttccgcccat
tctccgccccatggctgactaattttttttatttatgcagaggccgaggccgcctctgcctctgagct
attccagaagtagtgaggaggcttttttggaggcctaggcttttgcaaaaagctcccgggagcttgta
tatccattttcggatctgatcaagagacaggatgaggatcgtttcgcatgattgaacaagatggattg
cacgcaggttctccggccgcttgggtggagaggctattcggctatgactgggcacaacagacaatcgg
ctgctctgatgccgccgtgttccggctgtcagcgcaggggcgcccggttctttttgtcaagaccgacc
tgtccggtgccctgaatgaactgcaggacgaggcagcgcggctatcgtggctggccacgacgggcgtt
ccttgcgcagctgtgctcgacgttgtcactgaagcgggaagggactggctgctattgggcgaagtgcc
ggggcaggatctcctgtcatctcaccttgctcctgccgagaaagtatccatcatggctgatgcaatgc
ggcggctgcatacgcttgatccggctacctgcccattcgaccaccaagcgaaacatcgcatcgagcga
gcacgtactcggatggaagccggtcttgtcgatcaggatgatctggacgaagagcatcaggggctcgc
gccagccgaactgttcgccaggctcaaggcgcgcatgcccgacggcgaggatctcgtcgtgacccatg
gcgatgcctgcttgccgaatatcatggtggaaaatggccgcttttctggattcatcgactgtggccgg
ctgggtgtggcggaccgctatcaggacatagcgttggctacccgtgatattgctgaagagcttggcgg
cgaatgggctgaccgcttcctcgtgctttacggtatcgccgctcccgattcgcagcgcatcgccttct
atcgccttcttgacgagttcttctgagcgggactctggggttcgaaatgaccgaccaagcgacgccca
acctgccatcacgagatttcgattccaccgccgccttctatgaaaggttgggcttcggaatcgttttc
cgggacgccggctggatgatcctccagcgcggggatctcatgctggagttcttcgcccaccccaactt
gtttattgcagcttataatggttacaaataaagcaatagcatcacaaatttcacaaataaagcatttt
tttcactgcattctagttgtggtttgtccaaactcatcaatgtatcttatcatgtctgtataccgtcg
acctctagctagagcttggcgtaatcatggtcatagctgtttcctgtgtgaaattgttatccgctcac
aattccacacaacatacgagccggaagcataaagtgtaaagcctggggtgcctaatgagtgagctaac
tcacattaattgcgttgcgctcactgcccgctttccagtcgggaaacctgtcgtgccagctgcattaa
tgaatcggccaacgcgcggggagaggcggtttgcgtattgggcgctcttccgcttcctcgctcactga
ctcgctgcgctcggtcgttcggctgcggcgagcggtatcagctcactcaaaggcggtaatacggttat
ccacagaatcaggggataacgcaggaaagaacatgtgagcaaaaggccagcaaaaggccaggaaccgt
aaaaaggccgcgttgctggcgtttttccataggctccgcccccctgacgagcatcacaaaaatcgacg
ctcaagtcagaggtggcgaaacccgacaggactataaagataccaggcgtttccccctggaagctccc
tcgtgcgctctcctgttccgaccctgccgcttaccggatacctgtccgcctttctcccttcgggaagc
gtggcgctttctcatagctcacgctgtaggtatctcagttcggtgtaggtcgttcgctccaagctggg
ctgtgtgcacgaaccccccgttcagcccgaccgctgcgccttatccggtaactatcgtcttgagtcca
acccggtaagacacgacttatcgccactggcagcagccactggtaacaggattagcagagcgaggtat
gtaggcggtgctacagagttcttgaagtggtggcctaactacggctacactagaagaacagtatttgg
tatctgcgctctgctgaagccagttaccttcggaaaaagagttggtagctcttgatccggcaaacaaa
ccaccgctggtagcggtggtttttttgtttgcaagcagcagattacgcgcagaaaaaaaggatctcaa
gaagatcctttgatcttttctacggggtctgacgctcagtggaacgaaaactcacgttaagggatttt
ggtcatgagattatcaaaaaggatcttcacctagatccttttaaattaaaaatgaagttttaaatcaa
tctaaagtatatatgagtaaacttggtctgacagttaccaatgcttaatcagtgaggcacctatctca
gcgatctgtctatttcgttcatccatagttgcctgactccccgtcgtgtagataactacgatacggga
gggcttaccatctggccccagtgctgcaatgataccgcgagacccacgctcaccggctccagatttat
cagcaataaaccagccagccggaagggccgagcgcagaagtggtcctgcaactttatccgcctccatc
cagtctattaattgttgccgggaagctagagtaagtagttcgccagttaatagtttgcgcaacgttgt
tgccattgctacaggcatcgtggtgtcacgctcgtcgtttggtatggcttcattcagctccggttccc
aacgatcaaggcgagttacatgatcccccatgttgtgcaaaaaagcggttagctccttcggtcctccg
atcgttgtcagaagtaagttggccgcagtgttatcactcatggttatggcagcactgcataattctct
tactgtcatgccatccgtaagatgcttttctgtgactggtgagtactcaaccaagtcattctgagaat
agtgtatgcggcgaccgagttgctcttgcccggcgtcaatacgggataataccgcgccacatagcaga
actttaaaagtgctcatcattggaaaacgttcttcggggcgaaaactctcaaggatcttaccgctgtt
gagatccagttcgatgtaacccactcgtgcacccaactgatcttcagcatcttttactttcaccagcg
tttctgggtgagcaaaaacaggaaggcaaaatgccgcaaaaaagggaataagggcgacacggaaatgt
tgaatactcatactcttcctttttcaatattattgaagcatttatcagggttattgtctcatgagcgg
atacatatttgaatgtatttagaaaaataaacaaataggggttccgcgcacatttccccgaaaagtgc
cacctgacgtc
SA Hexapro Spike (pVP22b)
(SEQ ID NO: 65)
MFVFLVLLPLVSSQCVNFTTRTQLPPAYTNSFTRGVYYPDKVFRSSVLHSTQDLFLPFFSNVTWFHAI
HVSGTNGTKRFANPVLPFNDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNFKNLREFVFKNIDGYFKIY
SKHTPINLVRGLPQGFSALEPLVDLPIGINITRFQTLLALHISYLTPGDSSSGWTAGAAAYYVGYLQP
RTFLLKYNENGTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVYADSFVIRGDEVR
QIAPGQTGNIADYNYKLPDDFTGCVIAWNSNNLDSKVGGNYNYLYRLFRKSNLKPFERDISTEIYQAG
STPCNGVKGFNCYFPLQSYGFQPTYGVGYQPYRVVVLSFELLHAPATVCGPKKSTNLVKNKCVNFNFN
GLTGTGVLTESNKKFLPFQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECDIPIGAGICASYQTQTNS
PGSASSVASQSIIAYTMSLGVENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDFGGFNFSQILPDPSKPSKRS
PIEDLLFNKVTLADAGFIKQYGDCLGDIAARDLICAQKFNGLTVLPPLLTDEMIAQYTSALLAGTITS
GWTFGAGPALQIPFPMQMAYRFNGIGVTQNVLYENQKLIANQFNSAIGKIQDSLSSTPSALGKLQDVV
NQNAQALNTLVKQLSSNFGAISSVLNDILSRLDPPEAEVQIDRLITGRLQSLQTYVTQQLIRAAEIRA
SANLAATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT*
Indian B.1.617 Spike (pVP28)
(SEQ ID NO: 66)
MFVFLVLLPLVSSQCVNLTTRTQLPPAYTNSFTRGVYYPDKVFRSSVLHSTQDLFLPFFSNVTWFHAI
HVSGTNGTKRFDNPVLPFNDGVYFASTEKSNIIRGWIFGTTLDSKTQSLLIVNNATNVVIKVCEFQFC
NDPFLDVYYHKNNKSWMKSEFRVYSSANNCTFEYVSQPFLMDLEGKQGNFKNLREFVFKNIDGYFKIY
SKHTPINLVRDLPQGFSALEPLVDLPIGINITRFQTLLALHRSYLTPGDSSSGWTAGAAAYYVGYLQP
RTFLLKYNENGTITDAVDCALDPLSETKCTLKSFTVEKGIYQTSNERVQPTESIVRFPNITNLCPFGE
VFNATRFASVYAWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYRYRLFRKSNLKPFERDISTEIYQAG
STPCNGVQGFNCYFPLQSYGFQPTNGVGYQPYRVVVLSFELLHAPATVCGPKKSTNLVKNKCVNFNFN
GLTGTGVLTESNKKFLPFQQFGRDIADTTDAVRDPQTLEILDITPCSFGGVSVITPGTNTSNQVAVLY
QGVNCTEVPVAIHADQLTPTWRVYSTGSNVFQTRAGCLIGAEHVNNSYECDIPIGAGICASYQTQTNS
RRRARSVASQSIIAYTMSLGAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPLLTDEMIAQYTSALLAGTITS
GWTFGAGAALQIPFAMQMAYRFNGIGVTQNVLYENQKLIANQFNSAIGKIQDSLSSTASALGKLQDVV
NQNAQALNTLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAHEKNFTTAPAICHDGKAH
FPREGVFVSNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT*
Indian B.1.617 Spike Hexapro (pVP29b)
(SEQ ID NO: 67)
MFVFLVLLPLVSSQCVNLTTRTQLPPAYTNSFTRGVYYPDKVFRSSVLHSTQDLFLPFFSNVTWFHAI
HVSGTNGTKRFDNPVLPFNDGVYFASTEKSNIIRGWIFGTTLDSKTQSLLIVNNATNVVIKVCEFQFC
NDPFLDVYYHKNNKSWMKSEFRVYSSANNCTFEYVSQPFLMDLEGKQGNFKNLREFVFKNIDGYFKIY
SKHTPINLVRDLPQGFSALEPLVDLPIGINITRFQTLLALHRSYLTPGDSSSGWTAGAAAYYVGYLQP
RTFLLKYNENGTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYRYRLFRKSNLKPFERDISTEIYQAG
STPCNGVQGFNCYFPLQSYGFQPTNGVGYQPYRVVVLSFELLHAPATVCGPKKSTNLVKNKCVNFNFN
GLTGTGVLTESNKKFLPFQQFGRDIADTTDAVRDPQTLEILDITPCSFGGVSVITPGTNTSNQVAVLY
QGVNCTEVPVAIHADQLTPTWRVYSTGSNVFQTRAGCLIGAEHVNNSYECDIPIGAGICASYQTQTNS
RGSASSVASQSIIAYTMSLGAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDFGGFNFSQILPDPSKPSKRS
PIEDLLENKVTLADAGFIKQYGDCLGDIAARDLICAQKFNGLTVLPPLLTDEMIAQYTSALLAGTITS
GWTFGAGPALQIPFPMQMAYRFNGIGVTQNVLYENQKLIANQFNSAIGKIQDSLSSTPSALGKLQDVV
NQNAQALNTLVKQLSSNFGAISSVLNDILSRLDPPEAEVQIDRLITGRLQSLQTYVTQQLIRAAEIRA
SANLAATKMSECVLGQSKRVDFCGKGYHLMSFPQSAPHGVVFLHVTYVPAHEKNFTTAPAICHDGKAH
FPREGVFVSNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT*
Indian B.1.617 Spike Del-19 (pVP30)
(SEQ ID NO: 68)
MFVFLVLLPLVSSQCVNLTTRTQLPPAYTNSFTRGVYYPDKVFRSSVLHSTQDLFLPFFSNVTWFHAI
HVSGTNGTKRFDNPVLPENDGVYFASTEKSNIIRGWIFGTTLDSKTQSLLIVNNATNVVIKVCEFQFC
NDPFLDVYYHKNNKSWMKSEFRVYSSANNCTFEYVSQPFLMDLEGKQGNFKNLREFVFKNIDGYFKIY
SKHTPINLVRDLPQGFSALEPLVDLPIGINITRFQTLLALHRSYLTPGDSSSGWTAGAAAYYVGYLQP
RTFLLKYNENGTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYRYRLFRKSNLKPFERDISTEIYQAG
STPCNGVQGFNCYFPLQSYGFQPTNGVGYQPYRVVVLSFELLHAPATVCGPKKSTNLVKNKCVNFNFN
GLTGTGVLTESNKKFLPFQQFGRDIADTTDAVRDPQTLEILDITPCSFGGVSVITPGTNTSNQVAVLY
QGVNCTEVPVAIHADQLTPTWRVYSTGSNVFQTRAGCLIGAEHVNNSYECDIPIGAGICASYQTQTNS
RRRARSVASQSIIAYTMSLGAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPLLTDEMIAQYTSALLAGTITS
GWTFGAGAALQIPFAMQMAYRFNGIGVTQNVLYENQKLIANQFNSAIGKIQDSLSSTASALGKLQDVV
NQNAQALNTLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAHEKNFTTAPAICHDGKAH
FPREGVFVSNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQYIKWPWYIWLGFIAGL
IAIVMVTIMLCCMTSCCSCLKGCCSCGSCC*
SARS-COV-2 Spike Hexapro (pVP31b)
(SEQ ID NO: 69)
MFVFLVLLPLVSSQCVNLTTRTQLPPAYTNSFTRGVYYPDKVERSSVLHSTQDLFLPFFSNVTWFHAI
HVSGTNGTKRFDNPVLPFNDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNFKNLREFVFKNIDGYFKIY
SKHTPINLVRDLPQGFSALEPLVDLPIGINITRFQTLLALHRSYLTPGDSSSGWTAGAAAYYVGYLQP
RTFLLKYNENGTITDAVDCALDPLSETKCTLKSFTVEKGIYQTSNFRVQPTESIVRFPNITNLCPFGE
VFNATRFASVYAWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSNLKPFERDISTEIYQAG
STPCNGVEGFNCYFPLQSYGFQPTNGVGYQPYRVVVLSFELLHAPATVCGPKKSTNLVKNKCVNFNFN
GLTGTGVLTESNKKFLPFQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECDIPIGAGICASYQTQTNS
PGSASSVASQSIIAYTMSLGAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDFGGFNFSQILPDPSKPSKRS
PIEDLLFNKVTLADAGFIKQYGDCLGDIAARDLICAQKFNGLTVLPPLLTDEMIAQYTSALLAGTITS
GWTFGAGPALQIPFPMQMAYRFNGIGVTQNVLYENQKLIANQFNSAIGKIQDSLSSTPSALGKLQDVV
NQNAQALNTLVKQLSSNFGAISSVLNDILSRLDPPEAEVQIDRLITGRLQSLQTYVTQQLIRAAEIRA
SANLAATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT*

### Example 5: Development of an Omicron-Specific mRNA Vaccination Against SARS-CoV-2

### Materials and Methods:

Molecular cloning. The Omicron spike amino acid sequence was derived from two lineage BA.1 Omicron cases identified in Canada on Nov. 23, 2021 (GISAID EpiCoV, EPI_ISL_6826713 and EPI_ISL_6826714). Omicron spike cDNA were codon optimized, synthesized as gblocks (IDT) and cloned to mRNA vector with 5′, 3′ untranslated region (UTR) and poly A tail. The furin cleave site (RRAR) was replaced with a GSAS short stretch in the mRNA vector. HexaPro mutations were introduced in the WT sequence (Wuhan-Hu-1, which was used for the current clinical mRNA vaccines) and Omicron variant spike sequence of mRNA vector to improve expression and prefusion state. The accessory plasmids for pseudovirus assay including pHIVNLGagPol and pCCNanoLuc2AEGFP were from Dr. Bieniasz′ lab. The C-terminal 19 amino acids were deleted in the SARS-CoV-2 spike sequence for the pseudovirus assay. A list of oligos has been provided in table 2.

TABLE 2
List of oligos

| SEQ | | |
|---|---|---|
| ID | | |
| NO: | Name | Sequence |
| 23 | 46_gb0 | Gtgggaggtctatataagcagagctctctggctaac |
| | | tagagaacccactgcttactggcttatcgaaattaa |
| | | tacgactcactatagggagacccaagctggctagcc |
| | | accGAGAATAAACTAGTATTCTTCTGGTCCCCACAG |
| | | ACTCAGAGAGAACCCGCCACCATgTTTGTGTTCCTG |
| | | GTGCTGCTGCCACTGGTGTCCAGCCAGTGTGTGAAC |
| | | CTGACCACCAGGACCCAACTTCCTCCTGCCTACACC |
| | | AACTCCTTCACCAGGGGAGTCTACTACCCTGACAAG |
| | | GTGTTCAGGTCCTCTGTGCTGCACAGCACCCAGGAC |
| | | CTGTTCCTGCCATTCTTCAGCAATGTGACCTGGTTC |
| | | CATGTGATCTCTGGCACCAATGGCACCAAGAGGTTT |
| | | GACAACCCTGTGCTGCCATTCAATGATGGAGTCTAC |
| | | TTTGCCAGCAtcGAGAAGAGCAACATCATCAGGGGC |
| | | TGGATTTTTGGCACCACCCTGGACAGCAAGACCCAG |
| | | TCCCTGCTGATTGTGAACAATGCCACCAATGTGGTG |
| | | ATTAAGGTGTGTGAGTTCCAGTTCTGTAATGACCCA |
| | | TTCCTGGacCACAAGAACAACAAGTCCTGGATGG |
| 24 | 46_gb1 | CACAAGAACAACAAGTCCTGGATGGAGTCTGAGTTC |
| | | AGGGTCTACTCCTCTGCCAACAACTGTACCTTTGAA |
| | | TATGTGAGCCAACCATTCCTGATGGACTTGGAGGGC |
| | | AAGCAGGGCAACTTCAAGAACCTGAGGGAGTTTGTG |
| | | TTCAAGAACATTGATGGCTACTTCAAGATTTACAGC |
| | | AAACACACACCAATCatcGTGAGGGAGCCCGAGGAC |
| | | CTGCCACAGGGCTTCTCTGCCTTGGAACCACTGGTG |
| | | GACCTGCCAATTGGCATCAACATCACCAGGTTCCAG |
| | | ACCCTGCTGGCTCTGCACAGGTCCTACCTGACACCT |
| | | GGAGACTCCTCCTCTGGCTGGACAGCAGGAGCAGCA |
| | | GCCTACTATGTGGGCTACCTCCAACCAAGGACCTTC |
| | | CTGCTGAAATACAATGAGAATGGCACCATCACAGAT |
| | | GCTGTGGACTGTGCCCTGGACCCACTGTCTGAGACC |
| | | AAGTGTACCCTGAAATCCTTCACAGTGGAGAAGGGC |
| | | ATCTACCAGACCAGCAACTTCAGGGTCCAACCAACA |
| | | AGGAGCATTGTGAGGTTTCCAAACATCACCAACCTG |
| | | TGTCCATTTGacGAGGTGTTCAAcGCCACCAGGTTT |
| | | GCCTCTGTCTATGCCTGGAACAGG |
| 25 | 46_gb2 | GCCTCTGTCTATGCCTGGAACAGGAAGAGGATTAGC |
| | | AACTGTGTGGCTGACTACTCTGTGCTCTACAACctg |
| | | GCCcCCTTCttCACCTTCAAGTGTTATGGAGTGAGC |
| | | CCAACCAAACTGAATGACCTGTGTTTCACCAATGTC |
| | | TATGCTGACTCCTTTGTGATTAGGGGAGATGAGGTG |
| | | AGACAGATTGCCCCTGGACAAACAGGCAACATTGCT |
| | | GACTACAACTACAAACTGCCTGATGACTTCACAGGC |
| | | TGTGTGATTGCCTGGAACAGCAACAAgCTGGACAGC |
| | | AAGGTGagcGGCAACTACAACTACCTCTACAGACTG |
| | | TTCAGGAAGAGCAACCTGAAACCATTTGAGAGGGAC |
| | | ATCAGCACAGAGATTTACCAGGCTGGCAaCAagCCA |
| | | TGTAATGGAGTGGCCGGCTTCAACTGTTACTTTCCA |
| | | CTCCggTCCTATaGCTTCCggCCAACCtacGGAGTG |
| | | GGCcACCAACCATACAGGGTGGTGGTGCTGTCCTTT |
| | | GAACTGCTCCATGCCCCTGCCACAGTGTGTGGACCA |
| | | AAGAAGAGCACCAACCTGGTGAAGAACAAGTGTGTG |
| | | AACTTCAACTTCAATGGACTGAagGGCACAGGAGTG |
| | | CTGACAGAGAGCAACAAGAAGTTCCTGCCATTCCAA |
| | | CAGTTTGGCAGGGACATTGCTGACACCACAGATGCT |
| | | GTGAGGGACCCACAGACCTTGGAGATTCTGGACATC |
| | | ACACCATGTTCCTTTGGAGGAGTGTCTGTGATTACA |
| | | CCTGGCACCAACACCAGCAACCAGGTGGCTGTGCTC |
| | | TACCAGGgCGTGAACTGTACTGAGGTGCCTGTGGCT |
| | | ATCCATGCTGACCAACTTACACCAACCTGGAGGGTC |
| | | TACAGCACAGGCAGCAATGTGTTCCAGACCAGGGCT |
| | | GGCTGTCTGATTGGAGCAGAGtacGTGAACAACTCC |
| | | TATGAGTGTGACATCCCAATTGGAGCAGGCATCTG |
| 26 | 46_gb3 | CCCAATTGGAGCAGGCATCTGTGCCTCCTACCAGAC |
| | | CCAGACCAAgAGCCacGGCTCTGCATCTTCTGTGGC |
| | | AAGCCAGAGCATCATTGCCTACACAATGAGTCTGGG |
| | | AGCAGAGAACTCTGTGGCTTACAGCAACAACAGCAT |
| | | TGCCATCCCAACCAACTTCACCATCTCTGTGACCAC |
| | | AGAGATTCTGCCTGTGAGTATGACCAAGACCTCTGT |
| | | GGACTGTACAATGTATATCTGTGGAGACAGCACAGA |
| | | GTGTAGCAACCTGCTGCTCCAATATGGCTCCTTCTG |
| | | TACCCAACTTAAgAGGGCTCTGACAGGCATTGCTGT |
| | | GGAACAGGACAAGAACACCCAGGAGGTGTTTGCCCA |
| | | GGTGAAGCAGATTTACAAGACACCTCCAATCAAGtA |
| | | CTTTGGAGGCTTCAACTTCAGCCAGATTCTGCCTGA |
| | | CCCAAGCAAGCCAAGCAAGAGGTCCCCTATTGAGGA |
| | | CCTGCTGTTCAACAAGGTGACCCTGGCTGATGCTGG |
| | | CTTCATCAAGCAATATGGAGACTGTCTGGGAGACAT |
| | | TGCTGCCAGGGACCTGATTTGTGCCCAGAAGTTCAA |
| | | gGGACTGACAGTGCTGCCTCCACTGCTGACAGATGA |
| | | GATGATTGCCCAATACACCTCTGCCCTGCTGGCTGG |
| | | CACCATCACCTCTGGCTGGACCTTTGGAGCAGGACC |
| | | AGCCCTCCAAATCCCATTTCCAATGCAGATGGCTTA |
| | | CAGGTTCAATGGCATTGGAGTGACCCAGAATGTGCT |
| | | CTATGAGAACCAGAAACTGATTGCCAACCAGTTCAA |
| | | CTCTGCCATTGGCAAGATTCAGGACTCCCTGTCCAG |
| | | CACACCATCTGCCCTGGGCAAACTCCAAGATGTGGT |
| | | GAACCAcAATGCCCAGGCTCTGAACACCCTGGTGAA |
| | | GCAACTTTCCAGCAAgTTTGGAGCCATCTCCTCTGT |
| | | GCTGAATGACATCttcAGCAGACTGGACCCACCAGA |
| | | GGCTGAGGTCCAGATTGACAGACTG |
| 27 | 47_gb0 | cactatagggagacccaagctggctagccaccATgT |
| | | TTGTGTTCCTGGTGCTGCTGCCACT |
| | | GGTGTCCAGCCAGTGTGTGAACCTGACCACCAGGAC |
| | | CCAACTTCCTCCTGCCTACACCAACTCCTTCACCAG |
| | | GGGAGTCTACTACCCTGACAAGGTGTTCAGGTCCTC |
| | | TGTGCTGCACAGCACCCAGGACCTGTTCCTGCCATT |
| | | CTTCAGCAATGTGACCTGGTTCCATGTGATCTCTGG |
| | | CACCAATGGCACCAAGAGGTTTGACAACCCTGTGCT |
| | | GCCATTCAATGATGGAGTCTACTTTGCCAGCAtcGA |
| | | GAAGAGCAACATCATCAGGGGCTGGATTTTTGGCAC |
| | | CACCCTGGACAGCAAGACCCAGTCCCTGCTGATTGT |
| | | GAACAATGCCACCAATGTGGTGATTAAGGTGTGTGA |
| | | GTTCCAGTTCTGTAATGACCCATTCCTGGacCACAA |
| | | GAACAACAAGTCCTGGATG |
| 28 | 47_gb3 | CCCAATTGGAGCAGGCATCTGTGCCTCCTACCAGAC |
| | | CCAGACCAAgAGCCacAGGAGGGCAAGGTCTGTGGC |
| | | AAGCCAGAGCATCATTGCCTACACAATGAGTCTGGG |
| | | AGCAGAGAACTCTGTGGCTTACAGCAACAACAGCAT |
| | | TGCCATCCCAACCAACTTCACCATCTCTGTGACCAC |
| | | AGAGATTCTGCCTGTGAGTATGACCAAGACCTCTGT |
| | | GGACTGTACAATGTATATCTGTGGAGACAGCACAGA |
| | | GTGTAGCAACCTGCTGCTCCAATATGGCTCCTTCTG |
| | | TACCCAACTTAAgAGGGCTCTGACAGGCATTGCTGT |
| | | GGAACAGGACAAGAACACCCAGGAGGTGTTTGCCCA |
| | | GGTGAAGCAGATTTACAAGACACCTCCAATCAAGtA |
| | | CTTTGGAGGCTTCAACTTCAGCCAGATTCTGCCTGA |
| | | CCCAAGCAAGCCAAGCAAGAGGTCCTTCATTGAGGA |
| | | CCTGCTGTTCAACAAGGTGACCCTGGCTGATGCTGG |
| | | CTTCATCAAGCAATATGGAGACTGTCTGGGAGACAT |
| | | TGCTGCCAGGGACCTGATTTGTGCCCAGAAGTTCAA |
| | | gGGACTGACAGTGCTGCCTCCACTGCTGACAGATGA |
| | | GATGATTGCCCAATACACCTCTGCCCTGCTGGCTGG |
| | | CACCATCACCTCTGGCTGGACCTTTGGAGCAGGAGC |
| | | AGCCCTCCAAATCCCATTTGCTATGCAGATGGCTTA |
| | | CAGGTTCAATGGCATTGGAGTGACCCAGAATGTGCT |
| | | CTATGAGAACCAGAAACTGATTGCCAACCAGTTCAA |
| | | CTCTGCCATTGGCAAGATTCAGGACTCCCTGTCCAG |
| | | CACAGCCTCTGCCCTGGGCAAACTCCAAGATGTGGT |
| | | GAACCACAATGCCCAGGCTCTGAACACCCTGGTGAA |
| | | GCAACTTTCCAGCAAgTTTGGAGCCATCTCCTCTGT |
| | | GCTGAATGACATCttcAGCAGACTGGACAAGGTGGA |
| | | GGCTGAGGTCCAGATTGACAGACTG |
| 29 | CMV-F | CGCAAATGGGCGGTAGGCGTG |
| 30 | 47_F1 | GGGCTTCTCTGCCTTGGAACCAC |
| 31 | 47_F2 | GGCAACAAGCCATGTAATGGAGTG |
| 32 | 47_F3 | CTACACAATGAGTCTGGGAGCAGAG |

Cell Culture. HEK293T (ATCC CRL-3216), HEK293FT (Thermo Fisher Cat. No. R70007) and 293T-hACE2 (gifted from Dr Bieniasz′ lab) cell lines were maintained in Dulbecco's modified Eagle's medium (DMEM, Thermo fisher) supplemented with 10% Fetal bovine serum (Hyclone) and 1% penicillin-streptomycin (Gibco, final concentration penicillin 100 unit/ml, streptomycin 100 μg/ml), which is denoted as complete growth medium. Cells were split every 2 days at a split ratio of 1:4 when the confluency reached over 80%. Vero-E6 cells were cultured in Dulbecco's Modified Eagle Medium (DMEM) with 5% heat-inactivated fetal bovine serum (FBS).

In vitro mRNA transcription and vaccine formulation. A Hiscribe™ T7 ARCA mRNA Kit (with tailing) (NEB, Cat #E2060S) was used to in vitro transcribe codon-optimized mRNA encoding HexaPro spikes of SARS-CoV-2 WT and Omicron variant with 50% replacement of uridine by N1-methyl-pseudouridine. The DNA template was linearized before mRNA transcription and contained 5′ UTR, 3′ UTR and 3′polyA tail as flanking sequence of spike open reading frame.

The purified mRNA was generated by following NEB manufacturer's instructions and kept frozen at −80° C. until further use. The lipid nanoparticles mRNA was assembled using the NanoAssemblr® Ignite™ instrument (Precision Nanosystems) according to manufacturers' guidance. In brief, lipid mixture composed of 46.3% ALC-0315 (MedChemExpress, HY-138170), 1.6% ALC-0159 (MedChemExpress, HY-138300), 9.4% DSPC (Avanti polar lipids, 850365P) and 42.7% Cholesterol (Avanti polar lipids, 700100P), was mixed with prepared mRNA in 25 mM sodium acetate at pH 5.2 on Ignite instrument at a molar ratio of 6:1 (LNP: mRNA). The LNP encapsulated mRNA (LNP-mRNA) was buffer exchanged to PBS using 100 kDa Amicon filter (Macrosep Centrifugal Devices 100K, 89131-992). Sucrose was added as a cryoprotectant. The particle size of mRNA-LNP was determined by DLS device (DynaPro NanoStar, Wyatt, WDPN-06) and TEM described below. The encapsulation rate and mRNA concentration were quantified by Quant-iT™ RiboGreen™ RNA Assay (Thermo Fisher).

Validation of LNP-mRNA mediated spike expression in vitro and receptor binding capability of expressed Omicron HexaPro spikes. On day 1, HEK293T cells were seeded at 50% confluence in 24-well plate and mixed with 2 μg Omicron LNP-mRNA. After 16 hours, the cells were collected for flow cytometry. The spike expression on cell surface were detected by staining cells with human ACE2-Fc chimera (Sino Biological, 10108-H02HG) in MACS buffer (D-PBS with 2 mM EDTA and 0.5% BSA) for 20 min on ice. Cells were washed twice after the primary stain and incubated with PE-anti-human Fc antibody (Biolegend, Cat. No. 410708, Clone No. M1310G05, 1:100 dilution) in MACS buffer for 20 min on ice. During secondary antibody staining, live/Dead aqua fixable stain (Invitrogen) was used to assess cell viability. Data was collected on BD FACSAria II Cell Sorter (BD) and analyzed using FlowJo software (version 10.7.2, FlowJo LLC).

Negative-stain TEM. Formvar/carbon-coated copper grid (Electron Microscopy Sciences, catalog number FCF400-Cu-50) was glow-discharged and covered with 6 μl of the sample for 1 min before blotting away the sample. The sample was double-stained with 6 μl of 2% (w/v) uranyl formate (Electron Microscopy Sciences, catalog number 22450) for 5 seconds (first stain) and 1 min (second stain), blotting away after each stain. Images were collected using a JEOL JEM-1400 Plus microscope with an acceleration voltage of 80 kV and a bottom-mount charge-coupled device camera (4k by 3k, Advanced Microscopy Technologies).

Mouse vaccination. All experiments in this vaccine immunogenicity study used 6-8 weeks old female C57BL/6Ncr (B6) mice purchased from Charles River. The mice-housing condition was maintained at regular ambient room temperature (65-75° F., or 18-23° C.), 40-60% humidity, and a 14 h: 10 h day/night cycle. Each mice cage was individually ventilated with clean food, water, and bedding. Two sets of immunization experiments were performed: vaccination with Omicron LNP-mRNA, and sequential vaccination with WT LNP-mRNA, followed by WT or Omicron LNP mRNA booster. For the Omicron LNP-mRNA vaccination experiment, five mice were immunized with 10 μg Omicron LNP-mRNA on day 0 (prime) and day 14 (boost). Retro-orbital blood was collected prior to vaccine injection on day 0, day 13 and day 21. For WT and Omicron LNP-mRNA sequential vaccination experiment, 18 mice were administered with either 100 μl PBS (3+3 mice, two independent experiments) or two-dose 1 μg WT (on day 0 and day 21, 3+9 mice, two independent experiments) and 10 μg Omicron LNP-mRNA (over 3.5 months post prime). Retro-orbital blood was collected prior to vaccine injection on day 35, day 127, day 140 and day 148.

Isolation of plasma and PBMCs from blood. At the defined time points, retro-orbital blood was collected from mice. The isolation of PBMCs and plasma was achieved via centrifugation using SepMate-15 and Lymphoprep gradient medium (StemCell Technologies). 200 μl blood was immediately diluted with 800 μl PBS with 2% FBS. The blood diluent was then added to SepMate-15 tubes with 6 ml Lymphoprep (StemCell Technologies). Centrifugation at 1200 g for 20 minutes was used to isolate RBCs, PBMCs and plasma. 250 μl diluted plasma was collected from the surface layer. The remaining solution at the top layer was poured to a new tube to isolate PBMCs, which were washed once with PBS+2% FBS. The separated plasma was used in ELISA and neutralization assay.

ELISA. 3 μg/ml of spike antigens were coated onto the 384-well ELISA plates (VWR, Cat #82051-300) overnight at 4 degree. The antigen panel used in the ELISA includes RBDs of SARS RBD (AcroBiosystems, SPD-S52H6), MERS RBD (AcroBiosystems, SPD-M52H6), 2019-nCoV WA-1 (Sino Biological 40592-V08B), Delta variant B.1.617.2 (Sino Biological 40592-V08H90), Beta variant B.1.351 (Sino Biological 40592-V08H85) and Omicron variant B.1.1.529 (Sino Biological 40592-V08H121). Plates were washed with PBST (PBS plus 0.5% Tween 20) three times in the 50TS microplate washer (Fisher Scientific, NC0611021) and blocked with 0.5% BSA in PBST at room temperature for one hour. Plasma was fourfold serially diluted starting at a 1:500 dilution. Diluted plasma samples were added to the plates and incubated at room temperature for one hour, followed by washes with PBST five times. Anti-mouse secondary antibody (Fisher, Cat. No. A-10677) at 1:2500 dilution in blocking buffer was incubated at room temperature for one hour. Plates were washed five times and developed with tetramethylbenzidine substrate (Biolegend, 421101). The reaction was stopped with 1 M phosphoric acid after 20 min at room temperature, and OD at 450 nm was measured by multimode microplate reader (PerkinElmer EnVision 2105, Envision Manager v1.13.3009.1401). The binding response (OD450) was plotted against the dilution factor in log 10 scale as the dilution-dependent response curve. The area under curve of the dilution-dependent response (Log 10 AUC) was calculated to quantify the potency of the plasma antibody binding to spike antigens. The fold change of antibody titer was estimated using this equation: ratio=10\hat{ }(AUC1-AUC2).

hACE2 and antibody competition ELISA. The 384-well plate was coated with 0.6 μg/ml Omicron RBD at 4 degree overnight before washed with PBST (0.5% Tween-20) three times and blocked with 2% BSA in PBST for 1 hour at room temperature. In hACE2 and antibody competition ELISA, 15 μg/ml hACE2 (Sino, 10108-H08H) or 10 μg/ml antibodies including Clone 13A (Chen lab, in house), CR3022 (Abcam, Cat. No. Ab273073, Clone No. CR3022) and S309 (BioVision, Cat. No. A2266, Clone No. S309) were respectively added to the plate 1 hour prior to subsequent incubation with serially diluted plasma for another hour at room temperature. After coincubation of plasma and hACE2/antibodies, the plate was washed five times with PBST and incubated with anti-mouse secondary antibody with minimal cross reactivity with human IgG (Biolegend, Cat. No. 405306, Clone No. Poly4053, 1:2500 dilution). The plate was washed five times after 1-hour secondary antibody incubation and developed with tetramethylbenzidine substrate (Biolegend, 421101). The reaction was stopped with 1 M phosphoric acid after 20 min at room temperature, and OD at 450 nm was measured by multimode microplate reader (PerkinElmer En Vision 2105). The normalized AUC was calculated by normalizing the value with AUC determined in PBS group.

Omicron, WA-1 and Delta pseudovirus production and characterization. For the neutralization assay, HIV-1 based SARS-CoV-2 WA-1, B.1.617.2 (Delta) variant, and B.1.1.529 (Omicron) variant pseudotyped virions were packaged using a coronavirus spike plasmid, a reporter vector and a HIV-1 structural protein expression plasmid. The reporter vector, pCCNanoLuc2AEGFP, and plasmid expressing HIV-1 structural proteins (pHIVNLGagPol) were gifts from Dr Bieniasz's lab. The spike plasmid for SARS-CoV-2 WA-1 pseudovirus truncated C-terminal 19 amino acids (denoted as SARS-CoV-2-Δ19) and was from Dr Bieniasz′ lab. Spike plasmids expressing C-terminally truncated SARS-CoV-2 B.1.617.2 variant S protein (Delta variant-Δ19) and SARS-CoV-2 B.1.1.529 variant S protein (Omicron variant-Δ19) were made based on the pSARS-CoV-2-Δ19. All pseudoviruses were produced under the same conditions. Briefly, 293 FT cells were seeded in 150 mm plates, and transfected with 21 μg pHIVNLGagPol, 21 μg pCCNanoLuc2AEGFP, and 7.5 μg of corresponding plasmids, in the presence of 198 μl PEI (1 mg/ml, PEI MAX, Polyscience). At 48 h after transfection, the supernatant was filtered through a 0.45-μm filter, and frozen in −80° C.

To characterize the titer of WA-1, Delta, and Omicron pseudoviruses packaged, 1×104 293T-hACE2 cells were plated in each well of a 96-well plate. In the next day, different volumes of pseudovirus supplemented with culture medium to a total value of 100 μL were added into 96-well plates with 293T-hACE2. Plates were incubated at 37° C. for 24 hr. Then cells were washed with MACS buffer once and the percent of GFP-positive cells were counted by Attune NXT Acoustic Focusing Cytometer (Thermo Fisher, Attune N×T Software v3.1). To normalize pseudovirus titer, 1×104 293T-hACE2 cells were plated in each well of a 96-well plate. In the next day, 50 μL pseudovirus was mixed with 50 μL culture medium to 100 μL. The mixture was incubated for 1 hr in the 37° C. incubator, supplied with 5% CO2, and added into 96-well plates with 293T-hACE2. Plates were incubated at 37° C. for 24 hr. Then cells were washed with MACS buffer once and the percent of GFP-positive cells were counted by Attune NXT Acoustic Focusing Cytometer (Thermo Fisher). Delta pseudovirus and Omicron pseudovirus were diluted accordingly to match the functional titer of WA-1 pseudovirus for neutralization assay of plasma samples.

Pseudovirus neutralization assay. The SARS-CoV-2 pseudovirus assays were performed on 293T-hACE2 cells. One day before infection, 1×104 293T-hACE2 cells were plated in each well of a 96-well plate. In the next day, plasma collected from mice were serially diluted by 5 fold with complete growth medium at a starting dilution of 1:100. 55 μL diluted plasma was mixed with the same volume of SARS-CoV-2 WA-1, Delta variant, or Omicron variant pseudovirus and was incubated for 1 hr in the 37° C. incubator, supplied with 5% CO2. 100 μL of mixtures were added into 96-well plates with 293T-hACE2. Plates were incubated at 37° C. for 24 hr. Then cells were washed with MACS buffer once and the percent of GFP-positive cells were counted by Attune NXT Acoustic Focusing Cytometer (Thermo Fisher). The 50% inhibitory concentration (IC50) was calculated with a four-parameter logistic regression using GraphPad Prism (version 9.3.1, GraphPad Software Inc.). If the fitting value of IC50 is negative (i.e. negative titer), which suggested undetectable neutralization activity, the value was set to baseline (1, 0 in log scale).

Omicron and Delta live virus production and characterization. Full-length SARS-CoV-2 Omicron (BA.1) and Delta (B.1.617.2) isolates were a gift of Carolina Lucas and Akiko Iwasaki, and were isolated and sequenced. Remnant nasopharyngeal swap samples selected for virus isolation were diluted in DMEM by 10 fold and then filtered through a 45-μm filter. Tenfold serial dilution of samples was made from 1:50 to 1:19,531,250. The diluted samples were subsequently co-incubated with TMPRSS2-Vero E6 in a 96-well plate and adsorbed for 1 h at 37° C. Replacement medium was added after adsorption, and cells were incubated at 37° C. for up to 5 days. Supernatants from cells with cytopathic effect were collected, frozen, thawed and subjected to RT-qPCR.

To expand viral stocks, 107 Vero-E6 cells stably overexpressing ACE2 and TMPRSS2 were infected with SARS-CoV-2 at an MOI of approximately 0.01. The Omicron stock was collected 2 dpi, clarified by centrifugation (450×g for 10 minutes), filtered through a 0.45-micron filter, and concentrated ten-fold using Amicon Ultra-15 columns. To increase titer, the Delta stock was collected at 1 dpi, clarified, filtered, and used to infect 5×107 Vero-E6 cells overexpressing ACE2 and TMPRSS2. At 1 dpi, supernatant was harvested, clarified, filtered and concentrated as above. Viral stocks were titered by plaque assay in Vero-E6 cells. 7.5×105 and 4×105 Vero-E6 cells were seeded in each well of 6-well plates or 12-well plates. The media was replaced the next day with 100 μl of 10-fold serially diluted virus. Gentle rocking was applied to the plates incubated at 37° C. for 1 hour. Subsequently, overlay DMEM with 2% FBS and 0.6% Avicel RC-581 was added to each well. At 2 dpi for SARS-CoV-2, plates were fixed with 10% formaldehyde for 30 min, stained with crystal violet solution (0.5% crystal violet in 20% ethanol) for 30 min, and then rinsed with deionized water to visualize plaques.

Infectious virus neutralization assay. The complements and other potential neutralizing agents were heat inactivated in mouse plasma prior to infectious virus neutralization assay. Mouse plasma samples were serially diluted, then incubated with SARS-CoV-2 Omicron live virus for 1 h at 37° C. The Omicron live virus was isolated from nasopharyngeal specimens and sequenced as part of the Yale SARS-CoV-2 Genomic Surveillance Initiative's weekly surveillance Program in Connecticut65. After coincubation, plasma/virus mixture was added to Vero-E6 cells overexpressing ACE2/TMPRSS2. Cell viability was measured at 3 dpi or 5 dpi using CellTiter Glo.

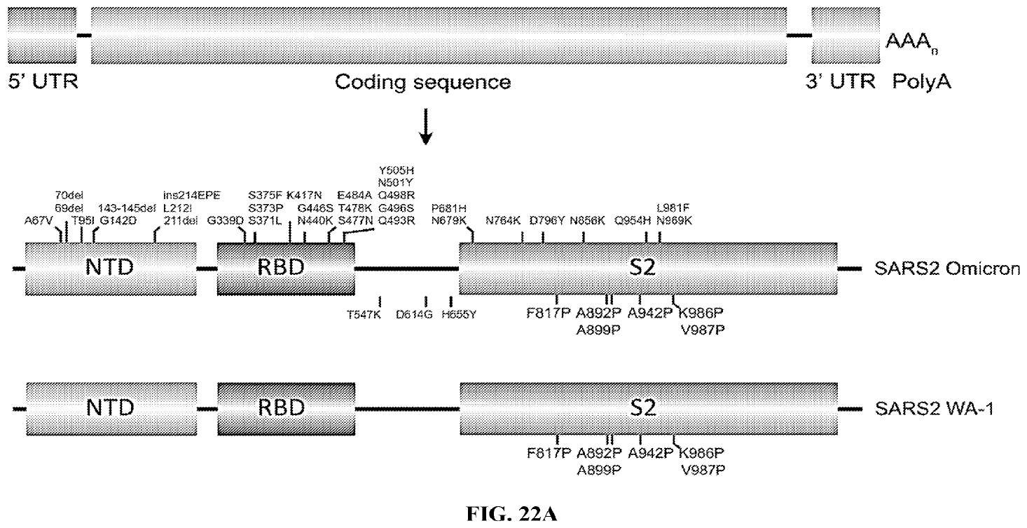

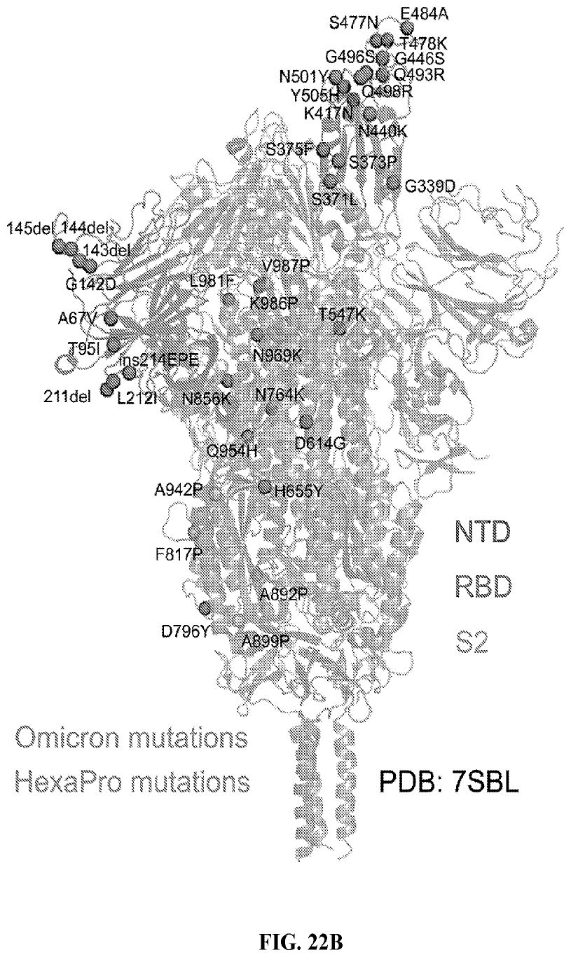

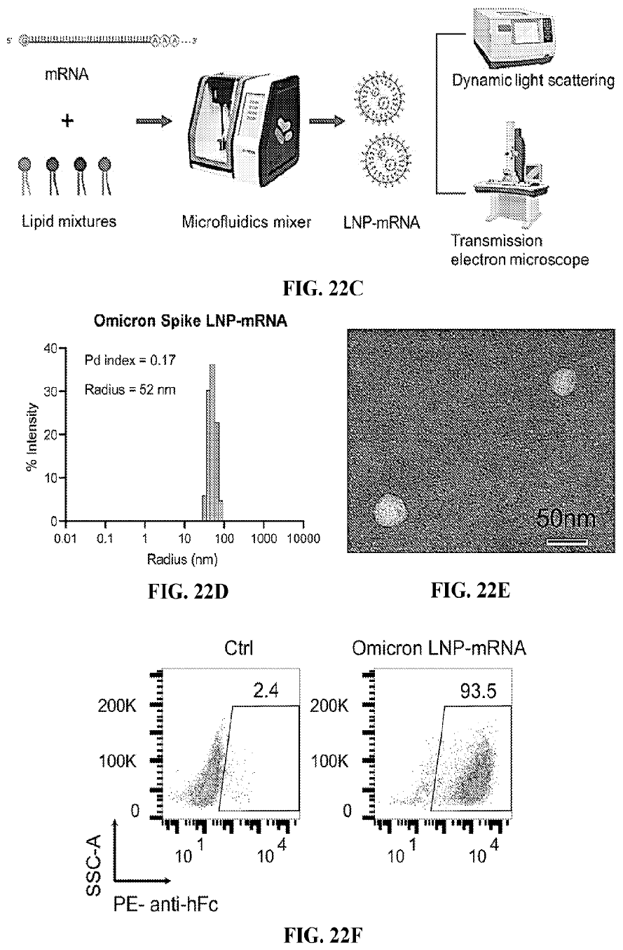

Statistics and Reproducibility. Standard statistical methods were applied to non-high-throughput experimental data. The statistical methods are described in here, figure legends and/or supplementary Excel tables. Data on dot-bar plots are shown as mean±s.e.m. with individual data points in plots. Two-way ANOVA with Tukey's multiple comparisons test and one-way ANOVA with Dunnett's multiple comparisons test were used to assess statistical significance for grouped and non-grouped datasets respectively. Statistical significance labels: * p<0.05; ** p<0.01; *** p<0.001; **** p<0.0001. Non-significant comparisons are not shown, unless otherwise noted as n.s., not significant. Sample number is designated as n from biologically independent samples. Prism (version 9.3.2, GraphPad Software Inc.) and RStudio (version 1.3.959, RStudio software company) were used for these analyses. Additional information can be found in the supplementary excel tables. Most of the data were collected from one independent experiment unless specifically stated otherwise in figure legends. Over 40 TEM micrographs were collected at various magnifications in one independent experiment and a representative micrograph was shown in FIG. 22.

pZF46 Omicron Spike Hexapro mRNA
(SEQ ID NO: 55)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATgTTTGTGTTCCTGGTGCTGCTGCCACTGGTGTCCAGCCAGTGTGTGAACCTGACCACCAG
GACCCAACTTCCTCCTGCCTACACCAACTCCTTCACCAGGGGAGTCTACTACCCTGACAAGGTGTTCA
GGTCCTCTGTGCTGCACAGCACCCAGGACCTGTTCCTGCCATTCTTCAGCAATGTGACCTGGTTCCAT
GTGATCTCTGGCACCAATGGCACCAAGAGGTTTGACAACCCTGTGCTGCCATTCAATGATGGAGTCTA
CTTTGCCAGCAtcGAGAAGAGCAACATCATCAGGGGCTGGATTTTTGGCACCACCCTGGACAGCAAGA
CCCAGTCCCTGCTGATTGTGAACAATGCCACCAATGTGGTGATTAAGGTGTGTGAGTTCCAGTTCTGT
AATGACCCATTCCTGGacCACAAGAACAACAAGTCCTGGATGGAGTCTGAGTTCAGGGTCTACTCCTC
TGCCAACAACTGTACCTTTGAATATGTGAGCCAACCATTCCTGATGGACTTGGAGGGCAAGCAGGGCA
ACTTCAAGAACCTGAGGGAGTTTGTGTTCAAGAACATTGATGGCTACTTCAAGATTTACAGCAAACAC
ACACCAATCatcGTGAGGGAGCCCGAGGACCTGCCACAGGGCTTCTCTGCCTTGGAACCACTGGTGGA
CCTGCCAATTGGCATCAACATCACCAGGTTCCAGACCCTGCTGGCTCTGCACAGGTCCTACCTGACAC
CTGGAGACTCCTCCTCTGGCTGGACAGCAGGAGCAGCAGCCTACTATGTGGGCTACCTCCAACCAAGG
ACCTTCCTGCTGAAATACAATGAGAATGGCACCATCACAGATGCTGTGGACTGTGCCCTGGACCCACT
GTCTGAGACCAAGTGTACCCTGAAATCCTTCACAGTGGAGAAGGGCATCTACCAGACCAGCAACTTCA
GGGTCCAACCAACAGAGAGCATTGTGAGGTTTCCAAACATCACCAACCTGTGTCCATTTGacGAGGTG
TTCAAcGCCACCAGGTTTGCCTCTGTCTATGCCTGGAACAGGAAGAGGATTAGCAACTGTGTGGCTGA
CTACTCTGTGCTCTACAACctgGCCcCCTTCttCACCTTCAAGTGTTATGGAGTGAGCCCAACCAAAC
TGAATGACCTGTGTTTCACCAATGTCTATGCTGACTCCTTTGTGATTAGGGGAGATGAGGTGAGACAG
ATTGCCCCTGGACAAACAGGCAACATTGCTGACTACAACTACAAACTGCCTGATGACTTCACAGGCTG
TGTGATTGCCTGGAACAGCAACAAgCTGGACAGCAAGGTGagcGGCAACTACAACTACCTCTACAGAC
TGTTCAGGAAGAGCAACCTGAAACCATTTGAGAGGGACATCAGCACAGAGATTTACCAGGCTGGCAaC
AagCCATGTAATGGAGTGGCCGGCTTCAACTGTTACTTTCCACTCCggTCCTATaGCTTCCggCCAAC
CtacGGAGTGGGCcACCAACCATACAGGGTGGTGGTGCTGTCCTTTGAACTGCTCCATGCCCCTGCCA
CAGTGTGTGGACCAAAGAAGAGCACCAACCTGGTGAAGAACAAGTGTGTGAACTTCAACTTCAATGGA
CTGAagGGCACAGGAGTGCTGACAGAGAGCAACAAGAAGTTCCTGCCATTCCAACAGTTTGGCAGGGA
CATTGCTGACACCACAGATGCTGTGAGGGACCCACAGACCTTGGAGATTCTGGACATCACACCATGTT
CCTTTGGAGGAGTGTCTGTGATTACACCTGGCACCAACACCAGCAACCAGGTGGCTGTGCTCTACCAG
GgcGTGAACTGTACTGAGGTGCCTGTGGCTATCCATGCTGACCAACTTACACCAACCTGGAGGGTCTA
CAGCACAGGCAGCAATGTGTTCCAGACCAGGGCTGGCTGTCTGATTGGAGCAGAGtacGTGAACAACT
CCTATGAGTGTGACATCCCAATTGGAGCAGGCATCTGTGCCTCCTACCAGACCCAGACCAAgAGCCac
GGCTCTGCATCTTCTGTGGCAAGCCAGAGCATCATTGCCTACACAATGAGTCTGGGAGCAGAGAACTC
TGTGGCTTACAGCAACAACAGCATTGCCATCCCAACCAACTTCACCATCTCTGTGACCACAGAGATTC
TGCCTGTGAGTATGACCAAGACCTCTGTGGACTGTACAATGTATATCTGTGGAGACAGCACAGAGTGT
AGCAACCTGCTGCTCCAATATGGCTCCTTCTGTACCCAACTTAAgAGGGCTCTGACAGGCATTGCTGT
GGAACAGGACAAGAACACCCAGGAGGTGTTTGCCCAGGTGAAGCAGATTTACAAGACACCTCCAATCA
AGtACTTTGGAGGCTTCAACTTCAGCCAGATTCTGCCTGACCCAAGCAAGCCAAGCAAGAGGTCCCCT
ATTGAGGACCTGCTGTTCAACAAGGTGACCCTGGCTGATGCTGGCTTCATCAAGCAATATGGAGACTG
TCTGGGAGACATTGCTGCCAGGGACCTGATTTGTGCCCAGAAGTTCAAgGGACTGACAGTGCTGCCTC
CACTGCTGACAGATGAGATGATTGCCCAATACACCTCTGCCCTGCTGGCTGGCACCATCACCTCTGGC
TGGACCTTTGGAGCAGGACCAGCCCTCCAAATCCCATTTCCAATGCAGATGGCTTACAGGTTCAATGG
CATTGGAGTGACCCAGAATGTGCTCTATGAGAACCAGAAACTGATTGCCAACCAGTTCAACTCTGCCA
TTGGCAAGATTCAGGACTCCCTGTCCAGCACACCATCTGCCCTGGGCAAACTCCAAGATGTGGTGAAC
CAcAATGCCCAGGCTCTGAACACCCTGGTGAAGCAACTTTCCAGCAAgTTTGGAGCCATCTCCTCTGT
GCTGAATGACATCttcAGCAGACTGGACCCACCAGAGGCTGAGGTCCAGATTGACAGACTGATTACAG
GCAGACTCCAATCCCTCCAAACCTATGTGACCCAACAACTTATCAGGGCTGCTGAGATTAGGGCATCT
GCCAACCTGGCTGCCACCAAGATGAGTGAGTGTGTGCTGGGACAAAGCAAGAGGGTGGACTTCTGTGG
CAAGGGCTACCACCTGATGAGTTTTCCACAGTCTGCCCCTCATGGAGTGGTGTTCCTGCATGTGACCT
ATGTGCCTGCCCAGGAGAAGAACTTCACCACAGCCCCTGCCATCTGCCATGATGGCAAGGCTCACTTT
CCAAGGGAGGGAGTGTTTGTGAGCAATGGCACCCACTGGTTTGTGACCCAGAGGAACTTCTATGAACC
ACAGATTATCACCACAGACAACACCTTTGTGTCTGGCAACTGTGATGTGGTGATTGGCATTGTGAACA
ACACAGTCTATGACCCACTCCAACCTGAACTGGACTCCTTCAAGGAGGAACTGGACAAATACTTCAAG
AACCACACCAGCCCTGATGTGGACCTGGGAGACATCTCTGGCATCAATGCCTCTGTGGTGAACATCCA
GAAGGAGATTGACAGACTGAATGAGGTGGCTAAGAACCTGAATGAGTCCCTGATTGACCTCCAAGAAC
TGGGCAAATATGAACAATACATCAAGTGGCCATGGTACATCTGGCTGGGCTTCATTGCTGGACTGATT
GCCATTGTGATGGTGACCATAATGCTGTGTTGTATGACCTCCTGTTGTTCCTGTCTGAAAGGCTGTTG
TTCCTGTGGCTCCTGTTGTAAGTTTGATGAGGATGACTCTGAACCTGTGCTGAAAGGAGTGAAACTGC
ACTACACCTGACTCGAGCTGGTACTGCATGCACGCAATGCTAGCTGCCCCTTTCCCGTCCTGGGTACC
CCGAGTCTCCCCCGACCTCGGGTCCCAGGTATGCTCCCACCTCCACCTGCCCCACTCACCACCTCTGC
TAGTTCCAGACACCTCCCAAGCACGCAGCAATGCAGCTCAAAACGCTTAGCCTAGCCACACCCCCACG
GGAAACAGCAGTGATTAACCTTTAGCAATAAACGAAAGTTTAACTAAGCTATACTAACCCCAGGGTTG
GTCAATTTCGTGCCAGCCACACCCTGGAGCTAGCaaaccgctgatcagcctcgactgtgccttctagt
tgccagccatctgttgtttgcccctcccccgtgccttccttgaccctggaaggtgccactcccactgt
cctttcctaataaaatgaggaaattgcatcgcattgtctgagtaggtgtcattctattctggggggtg
gggtggggcaggacagcaagggggaggattgggaagacaatagcaggcatgctggggatgcggtgggc
tctatggcttctgaggcggaaagaaccagctggggctctagggggtatccccacgcgccctgtagcgg
cgcattaagcgcggcgggtgtggtggttacgcgcagcgtgaccgctacacttgccagcgccctagcgc
ccgctcctttcgctttcttcccttcctttctcgccacgttcgccggctttccccgtcaagctctaaat
cgggggctccctttagggttccgatttagtgctttacggcacctcgaccccaaaaaacttgattaggg
tgatggttcacgtagtgggccatcgccctgatagacggtttttcgccctttgacgttggagtccacgt
tctttaatagtggactcttgttccaaactggaacaacactcaaccctatctcggtctattcttttgat
ttataagggattttgccgatttcggcctattggttaaaaaatgagctgatttaacaaaaatttaacgc
gaattaattctgtggaatgtgtgtcagttagggtgtggaaagtccccaggctccccagcaggcagaag
tatgcaaagcatgcatctcaattagtcagcaaccaggtgtggaaagtccccaggctccccagcaggca
gaagtatgcaaagcatgcatctcaattagtcagcaaccatagtcccgcccctaactccgcccatcccg
cccctaactccgcccagttccgcccattctccgccccatggctgactaattttttttatttatgcaga
ggccgaggccgcctctgcctctgagctattccagaagtagtgaggaggcttttttggaggcctaggct
tttgcaaaaagctcccgggagcttgtatatccattttcggatctgatcaagagacaggatgaggatcg
tttcgcatgattgaacaagatggattgcacgcaggttctccggccgcttgggtggagaggctattcgg
ctatgactgggcacaacagacaatcggctgctctgatgccgccgtgttccggctgtcagcgcaggggc
gcccggttctttttgtcaagaccgacctgtccggtgccctgaatgaactgcaggacgaggcagcgcgg
ctatcgtggctggccacgacgggcgttccttgcgcagctgtgctcgacgttgtcactgaagcgggaag
ggactggctgctattgggcgaagtgccggggcaggatctcctgtcatctcaccttgctcctgccgaga
aagtatccatcatggctgatgcaatgcggcggctgcatacgcttgatccggctacctgcccattcgac
caccaagcgaaacatcgcatcgagcgagcacgtactcggatggaagccggtcttgtcgatcaggatga
tctggacgaagagcatcaggggctcgcgccagccgaactgttcgccaggctcaaggcgcgcatgcccg
acggcgaggatctcgtcgtgacccatggcgatgcctgcttgccgaatatcatggtggaaaatggccgc
ttttctggattcatcgactgtggccggctgggtgtggcggaccgctatcaggacatagcgttggctac
ccgtgatattgctgaagagcttggcggcgaatgggctgaccgcttcctcgtgctttacggtatcgccg
ctcccgattcgcagcgcatcgccttctatcgccttcttgacgagttcttctgagcgggactctggggt
tcgaaatgaccgaccaagcgacgcccaacctgccatcacgagatttcgattccaccgccgccttctat
gaaaggttgggcttcggaatcgttttccgggacgccggctggatgatcctccagcgcggggatctcat
gctggagttcttcgcccaccccaacttgtttattgcagcttataatggttacaaataaagcaatagca
tcacaaatttcacaaataaagcatttttttcactgcattctagttgtggtttgtccaaactcatcaat
gtatcttatcatgtctgtataccgtcgacctctagctagagcttggcgtaatcatggtcatagctgtt
tcctgtgtgaaattgttatccgctcacaattccacacaacatacgagccggaagcataaagtgtaaag
cctggggtgcctaatgagtgagctaactcacattaattgcgttgcgctcactgcccgctttccagtcg
ggaaacctgtcgtgccagctgcattaatgaatcggccaacgcgcggggagaggcggtttgcgtattgg
gcgctcttccgcttcctcgctcactgactcgctgcgctcggtcgttcggctgcggcgagcggtatcag
ctcactcaaaggcggtaatacggttatccacagaatcaggggataacgcaggaaagaacatgtgagca
aaaggccagcaaaaggccaggaaccgtaaaaaggccgcgttgctggcgtttttccataggctccgccc
ccctgacgagcatcacaaaaatcgacgctcaagtcagaggtggcgaaacccgacaggactataaagat
accaggcgtttccccctggaagctccctcgtgcgctctcctgttccgaccctgccgcttaccggatac
ctgtccgcctttctcccttcgggaagcgtggcgctttctcatagctcacgctgtaggtatctcagttc
ggtgtaggtcgttcgctccaagctgggctgtgtgcacgaaccccccgttcagcccgaccgctgcgcct
tatccggtaactatcgtcttgagtccaacccggtaagacacgacttatcgccactggcagcagccact
ggtaacaggattagcagagcgaggtatgtaggcggtgctacagagttcttgaagtggtggcctaacta
cggctacactagaagaacagtatttggtatctgcgctctgctgaagccagttaccttcggaaaaagag
ttggtagctcttgatccggcaaacaaaccaccgctggtagcggtggtttttttgtttgcaagcagcag
attacgcgcagaaaaaaaggatctcaagaagatcctttgatcttttctacggggtctgacgctcagtg
gaacgaaaactcacgttaagggattttggtcatgagattatcaaaaaggatcttcacctagatccttt
taaattaaaaatgaagttttaaatcaatctaaagtatatatgagtaaacttggtctgacagttaccaa
tgcttaatcagtgaggcacctatctcagcgatctgtctatttcgttcatccatagttgcctgactccc
cgtcgtgtagataactacgatacgggagggcttaccatctggccccagtgctgcaatgataccgcgag
acccacgctcaccggctccagatttatcagcaataaaccagccagccggaagggccgagcgcagaagt
ggtcctgcaactttatccgcctccatccagtctattaattgttgccgggaagctagagtaagtagttc
gccagttaatagtttgcgcaacgttgttgccattgctacaggcatcgtggtgtcacgctcgtcgtttg
gtatggcttcattcagctccggttcccaacgatcaaggcgagttacatgatcccccatgttgtgcaaa
aaagcggttagctccttcggtcctccgatcgttgtcagaagtaagttggccgcagtgttatcactcat
ggttatggcagcactgcataattctcttactgtcatgccatccgtaagatgcttttctgtgactggtg
agtactcaaccaagtcattctgagaatagtgtatgcggcgaccgagttgctcttgcccggcgtcaata
cgggataataccgcgccacatagcagaactttaaaagtgctcatcattggaaaacgttcttcggggcg
aaaactctcaaggatcttaccgctgttgagatccagttcgatgtaacccactcgtgcacccaactgat
cttcagcatcttttactttcaccagcgtttctgggtgagcaaaaacaggaaggcaaaatgccgcaaaa
aagggaataagggcgacacggaaatgttgaatactcatactcttcctttttcaatattattgaagcat
ttatcagggttattgtctcatgagcggatacatatttgaatgtatttagaaaaataaacaaatagggg
ttccgcgcacatttccccgaaaagtgccacctgacgtc
pZF47 Omicron Spike Pseudovirus Del19
(SEQ ID NO: 56)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccATgTTTGTGTTCCTGGTGCTGCTGCCACTGGTGTCCAGCCAGTGTGTG
AACCTGACCACCAGGACCCAACTTCCTCCTGCCTACACCAACTCCTTCACCAGGGGAGTCTACTACCC
TGACAAGGTGTTCAGGTCCTCTGTGCTGCACAGCACCCAGGACCTGTTCCTGCCATTCTTCAGCAATG
TGACCTGGTTCCATGTGATCTCTGGCACCAATGGCACCAAGAGGTTTGACAACCCTGTGCTGCCATTC
AATGATGGAGTCTACTTTGCCAGCAtcGAGAAGAGCAACATCATCAGGGGCTGGATTTTTGGCACCAC
CCTGGACAGCAAGACCCAGTCCCTGCTGATTGTGAACAATGCCACCAATGTGGTGATTAAGGTGTGTG
AGTTCCAGTTCTGTAATGACCCATTCCTGGacCACAAGAACAACAAGTCCTGGATGGAGTCTGAGTTC
AGGGTCTACTCCTCTGCCAACAACTGTACCTTTGAATATGTGAGCCAACCATTCCTGATGGACTTGGA
GGGCAAGCAGGGCAACTTCAAGAACCTGAGGGAGTTTGTGTTCAAGAACATTGATGGCTACTTCAAGA
TTTACAGCAAACACACACCAATCatcGTGAGGGAGCCCGAGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGacGAGGTGTTCAAcGCCACCAGGTTTGCCTCTGTCTATGCCTGGAACAGGAAGAGGATTAGC
AACTGTGTGGCTGACTACTCTGTGCTCTACAACctgGCCcCCTTCttCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCAACATTGCTGACTACAACTACAAACTGCCTGAT
GACTTCACAGGCTGTGTGATTGCCTGGAACAGCAACAAgCTGGACAGCAAGGTGagcGGCAACTACAA
CTACCTCTACAGACTGTTCAGGAAGAGCAACCTGAAACCATTTGAGAGGGACATCAGCACAGAGATTT
ACCAGGCTGGCAaCAagCCATGTAATGGAGTGGccGGCTTCAACTGTTACTTTCCACTCCggTCCTAT
aGCTTCCggCCAACCtacGGAGTGGGCcACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGAagGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGgcGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GtacGTGAACAACTCCTATGAGTGTGACATCCCAATTGGAGCAGGCATCTGTGCCTCCTACCAGACCC
AGACCAAgAGCCacAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAAgAGGGCTCTG
ACAGGCATTGCTGTGGAACAGGACAAGAACACCCAGGAGGTGTTTGCCCAGGTGAAGCAGATTTACAA
GACACCTCCAATCAAGtACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAAgGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCACAATGCCCAGGCTCTGAACACCCTGGTGAAGCAACTTTCCAGCAAgTTTGGA
GCCATCTCCTCTGTGCTGAATGACATCttcAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTTGAggatccgagctcggtaccaagcttaagtttaa
accgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
acaatagcaggcatgctggggatgcggtgggctctatggcttctgaggcggaaagaaccagctggggc
tctagggggtatccccacgcgccctgtagcggcgcattaagcgcggcgggtgtggtggttacgcgcag
cgtgaccgctacacttgccagcgccctagcgcccgctcctttcgctttcttcccttcctttctcgcca
cgttcgccggctttccccgtcaagctctaaatcgggggctccctttagggttccgatttagtgcttta
cggcacctcgaccccaaaaaacttgattagggtgatggttcacgtagtgggccatcgccctgatagac
ggtttttcgccctttgacgttggagtccacgttctttaatagtggactcttgttccaaactggaacaa
cactcaaccctatctcggtctattcttttgatttataagggattttgccgatttcggcctattggtta
aaaaatgagctgatttaacaaaaatttaacgcgaattaattctgtggaatgtgtgtcagttagggtgt
ggaaagtccccaggctccccagcaggcagaagtatgcaaagcatgcatctcaattagtcagcaaccag
gtgtggaaagtccccaggctccccagcaggcagaagtatgcaaagcatgcatctcaattagtcagcaa
ccatagtcccgcccctaactccgcccatcccgcccctaactccgcccagttccgcccattctccgccc
catggctgactaattttttttatttatgcagaggccgaggccgcctctgcctctgagctattccagaa
gtagtgaggaggcttttttggaggcctaggcttttgcaaaaagctcccgggagcttgtatatccattt
tcggatctgatcaagagacaggatgaggatcgtttcgcatgattgaacaagatggattgcacgcaggt
tctccggccgcttgggtggagaggctattcggctatgactgggcacaacagacaatcggctgctctga
tgccgccgtgttccggctgtcagcgcaggggcgcccggttctttttgtcaagaccgacctgtccggtg
ccctgaatgaactgcaggacgaggcagcgcggctatcgtggctggccacgacgggcgttccttgcgca
gctgtgctcgacgttgtcactgaagcgggaagggactggctgctattgggcgaagtgccggggcagga
tctcctgtcatctcaccttgctcctgccgagaaagtatccatcatggctgatgcaatgcggcggctgc
atacgcttgatccggctacctgcccattcgaccaccaagcgaaacatcgcatcgagcgagcacgtact
cggatggaagccggtcttgtcgatcaggatgatctggacgaagagcatcaggggctcgcgccagccga
actgttcgccaggctcaaggcgcgcatgcccgacggcgaggatctcgtcgtgacccatggcgatgcct
gcttgccgaatatcatggtggaaaatggccgcttttctggattcatcgactgtggccggctgggtgtg
gcggaccgctatcaggacatagcgttggctacccgtgatattgctgaagagcttggcggcgaatgggc
tgaccgcttcctcgtgctttacggtatcgccgctcccgattcgcagcgcatcgccttctatcgccttc
ttgacgagttcttctgagcgggactctggggttcgaaatgaccgaccaagcgacgcccaacctgccat
cacgagatttcgattccaccgccgccttctatgaaaggttgggcttcggaatcgttttccgggacgcc
ggctggatgatcctccagcgcggggatctcatgctggagttcttcgcccaccccaacttgtttattgc
agcttataatggttacaaataaagcaatagcatcacaaatttcacaaataaagcatttttttcactgc
attctagttgtggtttgtccaaactcatcaatgtatcttatcatgtctgtataccgtcgacctctagc
tagagcttggcgtaatcatggtcatagctgtttcctgtgtgaaattgttatccgctcacaattccaca
caacatacgagccggaagcataaagtgtaaagcctggggtgcctaatgagtgagctaactcacattaa
ttgcgttgcgctcactgcccgctttccagtcgggaaacctgtcgtgccagctgcattaatgaatcggc
caacgcgcggggagaggcggtttgcgtattgggcgctcttccgcttcctcgctcactgactcgctgcg
ctcggtcgttcggctgcggcgagcggtatcagctcactcaaaggcggtaatacggttatccacagaat
caggggataacgcaggaaagaacatgtgagcaaaaggccagcaaaaggccaggaaccgtaaaaaggcc
gcgttgctggcgtttttccataggctccgcccccctgacgagcatcacaaaaatcgacgctcaagtca
gaggtggcgaaacccgacaggactataaagataccaggcgtttccccctggaagctccctcgtgcgct
ctcctgttccgaccctgccgcttaccggatacctgtccgcctttctcccttcgggaagcgtggcgctt
tctcatagctcacgctgtaggtatctcagttcggtgtaggtcgttcgctccaagctgggctgtgtgca
cgaaccccccgttcagcccgaccgctgcgccttatccggtaactatcgtcttgagtccaacccggtaa
gacacgacttatcgccactggcagcagccactggtaacaggattagcagagcgaggtatgtaggcggt
gctacagagttcttgaagtggtggcctaactacggctacactagaagaacagtatttggtatctgcgc
tctgctgaagccagttaccttcggaaaaagagttggtagctcttgatccggcaaacaaaccaccgctg
gtagcggtggtttttttgtttgcaagcagcagattacgcgcagaaaaaaaggatctcaagaagatcct
ttgatcttttctacggggtctgacgctcagtggaacgaaaactcacgttaagggattttggtcatgag
attatcaaaaaggatcttcacctagatccttttaaattaaaaatgaagttttaaatcaatctaaagta
tatatgagtaaacttggtctgacagttaccaatgcttaatcagtgaggcacctatctcagcgatctgt
ctatttcgttcatccatagttgcctgactccccgtcgtgtagataactacgatacgggagggcttacc
atctggccccagtgctgcaatgataccgcgagacccacgctcaccggctccagatttatcagcaataa
accagccagccggaagggccgagcgcagaagtggtcctgcaactttatccgcctccatccagtctatt
aattgttgccgggaagctagagtaagtagttcgccagttaatagtttgcgcaacgttgttgccattgc
tacaggcatcgtggtgtcacgctcgtcgtttggtatggcttcattcagctccggttcccaacgatcaa
ggcgagttacatgatcccccatgttgtgcaaaaaagcggttagctccttcggtcctccgatcgttgtc
agaagtaagttggccgcagtgttatcactcatggttatggcagcactgcataattctcttactgtcat
gccatccgtaagatgcttttctgtgactggtgagtactcaaccaagtcattctgagaatagtgtatgc
ggcgaccgagttgctcttgcccggcgtcaatacgggataataccgcgccacatagcagaactttaaaa
gtgctcatcattggaaaacgttcttcggggcgaaaactctcaaggatcttaccgctgttgagatccag
ttcgatgtaacccactcgtgcacccaactgatcttcagcatcttttactttcaccagcgtttctgggt
gagcaaaaacaggaaggcaaaatgccgcaaaaaagggaataagggcgacacggaaatgttgaatactc
atactcttcctttttcaatattattgaagcatttatcagggttattgtctcatgagcggatacatatt
tgaatgtatttagaaaaataaacaaataggggttccgcgcacatttccccgaaaagtgccacctgacg
tc
pZF56 Omicron BA.2 Spike Hexapro mRNA
(SEQ ID NO: 33)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATgTTTGTGTTCCTGGTGCTGCTGCCACTGGTGTCCAGCCAGTGTGTGAACCTGATCACCAG
GACCCAAAGCTACACCAACTCCTTCACCAGGGGAGTCTACTACCCTGACAAGGTGTTCAGGTCCTCTG
TGCTGCACAGCACCCAGGACCTGTTCCTGCCATTCTTCAGCAATGTGACCTGGTTCCATGCCATCCAC
GTGTCTGGCACCAATGGCACCAAGAGGTTTGACAACCCTGTGCTGCCATTCAATGATGGAGTCTACTT
TGCCAGCACCGAGAAGAGCAACATCATCAGGGGCTGGATTTTTGGCACCACCCTGGACAGCAAGACCC
AGTCCCTGCTGATTGTGAACAATGCCACCAATGTGGTGATTAAGGTGTGTGAGTTCCAGTTCTGTAAT
GACCCATTCCTGGacGTGTACTACCACAAGAACAACAAGTCCTGGATGGAGTCTGAGTTCAGGGTCTA
CTCCTCTGCCAACAACTGTACCTTTGAATATGTGAGCCAACCATTCCTGATGGACTTGGAGGGCAAGC
AGGGCAACTTCAAGAACCTGAGGGAGTTTGTGTTCAAGAACATTGATGGCTACTTCAAGATTTACAGC
AAACACACACCAATCAACCTGGGCAGAGACCTGCCACAGGGCTTCTCTGCCTTGGAACCACTGGTGGA
CCTGCCAATTGGCATCAACATCACCAGGTTCCAGACCCTGCTGGCTCTGCACAGGTCCTACCTGACAC
CTGGAGACTCCTCCTCTGGCTGGACAGCAGGAGCAGCAGCCTACTATGTGGGCTACCTCCAACCAAGG
ACCTTCCTGCTGAAATACAATGAGAATGGCACCATCACAGATGCTGTGGACTGTGCCCTGGACCCACT
GTCTGAGACCAAGTGTACCCTGAAATCCTTCACAGTGGAGAAGGGCATCTACCAGACCAGCAACTTCA
GGGTCCAACCAACAGAGAGCATTGTGAGGTTTCCAAACATCACCAACCTGTGTCCATTTGacGAGGTG
TTCAAcGCCACCAGGTTTGCCTCTGTCTATGCCTGGAACAGGAAGAGGATTAGCAACTGTGTGGCTGA
CTACTCTGTGCTCTACAACTTCGCCcCCTTCttCGCCTTCAAGTGTTATGGAGTGAGCCCAACCAAAC
TGAATGACCTGTGTTTCACCAATGTCTATGCTGACTCCTTTGTGATTAGGGGAAACGAGGTGAGCCAG
ATTGCCCCTGGACAAACAGGCAAcATTGCTGACTACAACTACAAACTGCCTGATGACTTCACAGGCTG
TGTGATTGCCTGGAACAGCAACAAgCTGGACAGCAAGGTGGGCGGCAACTACAACTACCTCTACAGAC
TGTTCAGGAAGAGCAACCTGAAACCATTTGAGAGGGACATCAGCACAGAGATTTACCAGGCTGGCAaC
AagCCATGTAATGGAGTGGCCGGCTTCAACTGTTACTTTCCACTCCggTCCTATGGCTTCCggCCAAC
CtacGGAGTGGGCCACCAACCATACAGGGTGGTGGTGCTGTCCTTTGAACTGCTCCATGCCCCTGCCA
CAGTGTGTGGACCAAAGAAGAGCACCAACCTGGTGAAGAACAAGTGTGTGAACTTCAACTTCAATGGA
CTGACCGGCACAGGAGTGCTGACAGAGAGCAACAAGAAGTTCCTGCCATTCCAACAGTTTGGCAGGGA
CATTGCTGACACCACAGATGCTGTGAGGGACCCACAGACCTTGGAGATTCTGGACATCACACCATGTT
CCTTTGGAGGAGTGTCTGTGATTACACCTGGCACCAACACCAGCAACCAGGTGGCTGTGCTCTACCAG
GgcGTGAACTGTACTGAGGTGCCTGTGGCTATCCATGCTGACCAACTTACACCAACCTGGAGGGTCTA
CAGCACAGGCAGCAATGTGTTCCAGACCAGGGCTGGCTGTCTGATTGGAGCAGAGtacGTGAACAACT
CCTATGAGTGTGACATCCCAATTGGAGCAGGCATCTGTGCCTCCTACCAGACCCAGACCAAgAGCCac
AGGAGGGCAAGGTCTGTGGCAAGCCAGAGCATCATTGCCTACACAATGAGTCTGGGAGCAGAGAACTC
TGTGGCTTACAGCAACAACAGCATTGCCATCCCAACCAACTTCACCATCTCTGTGACCACAGAGATTC
TGCCTGTGAGTATGACCAAGACCTCTGTGGACTGTACAATGTATATCTGTGGAGACAGCACAGAGTGT
AGCAACCTGCTGCTCCAATATGGCTCCTTCTGTACCCAACTTAAgAGGGCTCTGACAGGCATTGCTGT
GGAACAGGACAAGAACACCCAGGAGGTGTTTGCCCAGGTGAAGCAGATTTACAAGACACCTCCAATCA
AGtACTTTGGAGGCTTCAACTTCAGCCAGATTCTGCCTGACCCAAGCAAGCCAAGCAAGAGGTCCCCT
ATTGAGGACCTGCTGTTCAACAAGGTGACCCTGGCTGATGCTGGCTTCATCAAGCAATATGGAGACTG
TCTGGGAGACATTGCTGCCAGGGACCTGATTTGTGCCCAGAAGTTCAACGGACTGACAGTGCTGCCTC
CACTGCTGACAGATGAGATGATTGCCCAATACACCTCTGCCCTGCTGGCTGGCACCATCACCTCTGGC
TGGACCTTTGGAGCAGGACCAGCCCTCCAAATCCCATTTCCAATGCAGATGGCTTACAGGTTCAATGG
CATTGGAGTGACCCAGAATGTGCTCTATGAGAACCAGAAACTGATTGCCAACCAGTTCAACTCTGCCA
TTGGCAAGATTCAGGACTCCCTGTCCAGCACACCATCTGCCCTGGGCAAGCTCCAAGATGTGGTGAAC
CAcAATGCCCAGGCTCTGAACACCCTGGTGAAGCAACTTTCCAGCAAgTTTGGAGCCATCTCCTCTGT
GCTGAATGACATCCTGAGCAGACTGGACCCACCAGAGGCTGAGGTCCAGATTGACAGACTGATTACAG
GCAGACTCCAATCCCTCCAAACCTATGTGACCCAACAACTTATCAGGGCTGCTGAGATTAGGGCATCT
GCCAACCTGGCTGCCACCAAGATGAGTGAGTGTGTGCTGGGACAAAGCAAGAGGGTGGACTTCTGTGG
CAAGGGCTACCACCTGATGAGTTTTCCACAGTCTGCCCCTCATGGAGTGGTGTTCCTGCATGTGACCT
ATGTGCCTGCCCAGGAGAAGAACTTCACCACAGCCCCTGCCATCTGCCATGATGGCAAGGCTCACTTT
CCAAGGGAGGGAGTGTTTGTGAGCAATGGCACCCACTGGTTTGTGACCCAGAGGAACTTCTATGAACC
ACAGATTATCACCACAGACAACACCTTTGTGTCTGGCAACTGTGATGTGGTGATTGGCATTGTGAACA
ACACAGTCTATGACCCACTCCAACCTGAACTGGACTCCTTCAAGGAGGAACTGGACAAATACTTCAAG
AACCACACCAGCCCTGATGTGGACCTGGGAGACATCTCTGGCATCAATGCCTCTGTGGTGAACATCCA
GAAGGAGATTGACAGACTGAATGAGGTGGCTAAGAACCTGAATGAGTCCCTGATTGACCTCCAAGAAC
TGGGCAAATATGAACAATACATCAAGTGGCCATGGTACATCTGGCTGGGCTTCATTGCTGGACTGATT
GCCATTGTGATGGTGACCATAATGCTGTGTTGTATGACCTCCTGTTGTTCCTGTCTGAAAGGCTGTTG
TTCCTGTGGCTCCTGTTGTAAGTTTGATGAGGATGACTCTGAACCTGTGCTGAAAGGAGTGAAACTGC
ACTACACCTGACTCGAGCTGGTACTGCATGCACGCAATGCTAGCTGCCCCTTTCCCGTCCTGGGTACC
CCGAGTCTCCCCCGACCTCGGGTCCCAGGTATGCTCCCACCTCCACCTGCCCCACTCACCACCTCTGC
TAGTTCCAGACACCTCCCAAGCACGCAGCAATGCAGCTCAAAACGCTTAGCCTAGCCACACCCCCACG
GGAAACAGCAGTGATTAACCTTTAGCAATAAACGAAAGTTTAACTAAGCTATACTAACCCCAGGGTTG
GTCAATTTCGTGCCAGCCACACCCTGGAGCTAGCaaaccgctgatcagcctcgactgtgccttctagt
tgccagccatctgttgtttgcccctcccccgtgccttccttgaccctggaaggtgccactcccactgt
cctttcctaataaaatgaggaaattgcatcgcattgtctgagtaggtgtcattctattctggggggtg
gggtggggcaggacagcaagggggaggattgggaagacaatagcaggcatgctggggatgcggtgggc
tctatggcttctgaggcggaaagaaccagctggggctctagggggtatccccacgcgccctgtagcgg
cgcattaagcgcggcgggtgtggtggttacgcgcagcgtgaccgctacacttgccagcgccctagcgc
ccgctcctttcgctttcttcccttcctttctcgccacgttcgccggctttccccgtcaagctctaaat
cgggggctccctttagggttccgatttagtgctttacggcacctcgaccccaaaaaacttgattaggg
tgatggttcacgtagtgggccatcgccctgatagacggtttttcgccctttgacgttggagtccacgt
tctttaatagtggactcttgttccaaactggaacaacactcaaccctatctcggtctattcttttgat
ttataagggattttgccgatttcggcctattggttaaaaaatgagctgatttaacaaaaatttaacgc
gaattaattctgtggaatgtgtgtcagttagggtgtggaaagtccccaggctccccagcaggcagaag
tatgcaaagcatgcatctcaattagtcagcaaccaggtgtggaaagtccccaggctccccagcaggca
gaagtatgcaaagcatgcatctcaattagtcagcaaccatagtcccgcccctaactccgcccatcccg
cccctaactccgcccagttccgcccattctccgccccatggctgactaattttttttatttatgcaga
ggccgaggccgcctctgcctctgagctattccagaagtagtgaggaggcttttttggaggcctaggct
tttgcaaaaagctcccgggagcttgtatatccattttcggatctgatcaagagacaggatgaggatcg
tttcgcatgattgaacaagatggattgcacgcaggttctccggccgcttgggtggagaggctattcgg
ctatgactgggcacaacagacaatcggctgctctgatgccgccgtgttccggctgtcagcgcaggggc
gcccggttctttttgtcaagaccgacctgtccggtgccctgaatgaactgcaggacgaggcagcgcgg
ctatcgtggctggccacgacgggcgttccttgcgcagctgtgctcgacgttgtcactgaagcgggaag
ggactggctgctattgggcgaagtgccggggcaggatctcctgtcatctcaccttgctcctgccgaga
aagtatccatcatggctgatgcaatgcggcggctgcatacgcttgatccggctacctgcccattcgac
caccaagcgaaacatcgcatcgagcgagcacgtactcggatggaagccggtcttgtcgatcaggatga
tctggacgaagagcatcaggggctcgcgccagccgaactgttcgccaggctcaaggcgcgcatgcccg
acggcgaggatctcgtcgtgacccatggcgatgcctgcttgccgaatatcatggtggaaaatggccgc
ttttctggattcatcgactgtggccggctgggtgtggcggaccgctatcaggacatagcgttggctac
ccgtgatattgctgaagagcttggcggcgaatgggctgaccgcttcctcgtgctttacggtatcgccg
ctcccgattcgcagcgcatcgccttctatcgccttcttgacgagttcttctgagcgggactctggggt
tcgaaatgaccgaccaagcgacgcccaacctgccatcacgagatttcgattccaccgccgccttctat
gaaaggttgggcttcggaatcgttttccgggacgccggctggatgatcctccagcgcggggatctcat
gctggagttcttcgcccaccccaacttgtttattgcagcttataatggttacaaataaagcaatagca
tcacaaatttcacaaataaagcatttttttcactgcattctagttgtggtttgtccaaactcatcaat
gtatcttatcatgtctgtataccgtcgacctctagctagagcttggcgtaatcatggtcatagctgtt
tcctgtgtgaaattgttatccgctcacaattccacacaacatacgagccggaagcataaagtgtaaag
cctggggtgcctaatgagtgagctaactcacattaattgcgttgcgctcactgcccgctttccagtcg
ggaaacctgtcgtgccagctgcattaatgaatcggccaacgcgcggggagaggcggtttgcgtattgg
gcgctcttccgcttcctcgctcactgactcgctgcgctcggtcgttcggctgcggcgagcggtatcag
ctcactcaaaggcggtaatacggttatccacagaatcaggggataacgcaggaaagaacatgtgagca
aaaggccagcaaaaggccaggaaccgtaaaaaggccgcgttgctggcgtttttccataggctccgccc
ccctgacgagcatcacaaaaatcgacgctcaagtcagaggtggcgaaacccgacaggactataaagat
accaggcgtttccccctggaagctccctcgtgcgctctcctgttccgaccctgccgcttaccggatac
ctgtccgcctttctcccttcgggaagcgtggcgctttctcatagctcacgctgtaggtatctcagttc
ggtgtaggtcgttcgctccaagctgggctgtgtgcacgaaccccccgttcagcccgaccgctgcgcct
tatccggtaactatcgtcttgagtccaacccggtaagacacgacttatcgccactggcagcagccact
ggtaacaggattagcagagcgaggtatgtaggcggtgctacagagttcttgaagtggtggcctaacta
cggctacactagaagaacagtatttggtatctgcgctctgctgaagccagttaccttcggaaaaagag
ttggtagctcttgatccggcaaacaaaccaccgctggtagcggtggtttttttgtttgcaagcagcag
attacgcgcagaaaaaaaggatctcaagaagatcctttgatcttttctacggggtctgacgctcagtg
gaacgaaaactcacgttaagggattttggtcatgagattatcaaaaaggatcttcacctagatccttt
taaattaaaaatgaagttttaaatcaatctaaagtatatatgagtaaacttggtctgacagttaccaa
tgcttaatcagtgaggcacctatctcagcgatctgtctatttcgttcatccatagttgcctgactccc
cgtcgtgtagataactacgatacgggagggcttaccatctggccccagtgctgcaatgataccgcgag
acccacgctcaccggctccagatttatcagcaataaaccagccagccggaagggccgagcgcagaagt
ggtcctgcaactttatccgcctccatccagtctattaattgttgccgggaagctagagtaagtagttc
gccagttaatagtttgcgcaacgttgttgccattgctacaggcatcgtggtgtcacgctcgtcgtttg
gtatggcttcattcagctccggttcccaacgatcaaggcgagttacatgatcccccatgttgtgcaaa
aaagcggttagctccttcggtcctccgatcgttgtcagaagtaagttggccgcagtgttatcactcat
ggttatggcagcactgcataattctcttactgtcatgccatccgtaagatgcttttctgtgactggtg
agtactcaaccaagtcattctgagaatagtgtatgcggcgaccgagttgctcttgcccggcgtcaata
cgggataataccgcgccacatagcagaactttaaaagtgctcatcattggaaaacgttcttcggggcg
aaaactctcaaggatcttaccgctgttgagatccagttcgatgtaacccactcgtgcacccaactgat
cttcagcatcttttactttcaccagcgtttctgggtgagcaaaaacaggaaggcaaaatgccgcaaaa
aagggaataagggcgacacggaaatgttgaatactcatactcttcctttttcaatattattgaagcat
ttatcagggttattgtctcatgagcggatacatatttgaatgtatttagaaaaataaacaaatagggg
ttccgcgcacatttccccgaaaagtgccacctgacgtc
Omicron BA.2 Spike Hexapro
(SEQ ID NO: 34)
MFVFLVLLPLVSSQCVNLITRTQSYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIKVCEFQFCNDP
FLDVYYHKNNKSWMESEFRVYSSANNCTFEYVSQPFLMDLEGKQGNFKNLREFVFKNIDGYFKIYSKH
TPINLGRDLPQGFSALEPLVDLPIGINITRFQTLLALHRSYLTPGDSSSGWTAGAAAYYVGYLQPRTF
LLKYNENGTITDAVDCALDPLSETKCTLKSFTVEKGIYQTSNFRVQPTESIVRFPNITNLCPFDEVFN
ATRFASVYAWNRKRISNCVADYSVLYNFAPFFAFKCYGVSPTKLNDLCFTNVYADSFVIRGNEVSQIA
PGQTGNIADYNYKLPDDFTGCVIAWNSNKLDSKVGGNYNYLYRLFRKSNLKPFERDISTEIYQAGNKP
CNGVAGFNCYFPLRSYGFRPTYGVGHQPYRVVVLSFELLHAPATVCGPKKSTNLVKNKCVNFNFNGLT
GTGVLTESNKKFLPFQQFGRDIADTTDAVRDPQTLEILDITPCSFGGVSVITPGTNTSNQVAVLYQGV
NCTEVPVAIHADQLTPTWRVYSTGSNVFQTRAGCLIGAEYVNNSYECDIPIGAGICASYQTQTKSHRR
ARSVASQSIIAYTMSLGAENSVAYSNNSIAIPTNFTISVTTEILPVSMTKTSVDCTMYICGDSTECSN
LLLQYGSFCTQLKRALTGIAVEQDKNTQEVFAQVKQIYKTPPIKYFGGFNFSQILPDPSKPSKRSPIE
DLLFNKVTLADAGFIKQYGDCLGDIAARDLICAQKFNGLTVLPPLLTDEMIAQYTSALLAGTITSGWT
FGAGPALQIPFPMQMAYRFNGIGVTQNVLYENQKLIANQFNSAIGKIQDSLSSTPSALGKLQDVVNHN
AQALNTLVKQLSSKFGAISSVLNDILSRLDPPEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICHDGKAHFPR
EGVFVSNGTHWFVTQRNFYEPQIITTDNTFVSGNCDVVIGIVNNTVYDPLQPELDSFKEELDKYFKNH
TSPDVDLGDISGINASVVNIQKEIDRLNEVAKNLNESLIDLQELGKYEQYIKWPWYIWLGFIAGLIAI
VMVTIMLCCMTSCCSCLKGCCSCGSCCKFDEDDSEPVLKGVKLHYT*
pZF57 Omicron BA.2 Spike Pseudovirus del 19
(SEQ ID NO: 35)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccATgTTTGTGTTCCTGGTGCTGCTGCCACTGGTGTCCAGCCAGTGTGTG
AACCTGATCACCAGGACCCAAAGCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCACGTGTCTGGCACCAATGGCACCAAGAGGTTTGACAACCCTGTGCTGCCATTCAAT
GATGGAGTCTACTTTGCCAGCACCGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGacGTGTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGGCAGAGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGacGAGGTGTTCAAcGCCACCAGGTTTGCCTCTGTCTATGCCTGGAACAGGAAGAGGATTAGC
AACTGTGTGGCTGACTACTCTGTGCTCTACAACTTCGCCcCCTTCttCGCCTTCAAGTGTTATGGAGT
GAGCCCAACCAAACTGAATGACCTGTGTTTCACCAATGTCTATGCTGACTCCTTTGTGATTAGGGGAA
ACGAGGTGAGCCAGATTGCCCCTGGACAAACAGGCAAcATTGCTGACTACAACTACAAACTGCCTGAT
GACTTCACAGGCTGTGTGATTGCCTGGAACAGCAACAAgCTGGACAGCAAGGTGGGCGGCAACTACAA
CTACCTCTACAGACTGTTCAGGAAGAGCAACCTGAAACCATTTGAGAGGGACATCAGCACAGAGATTT
ACCAGGCTGGCAaCAagCCATGTAATGGAGTGGccGGCTTCAACTGTTACTTTCCACTCCggTCCTAT
GGCTTCCggCCAACCtacGGAGTGGGCcACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACCGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGgcGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GtacGTGAACAACTCCTATGAGTGTGACATCCCAATTGGAGCAGGCATCTGTGCCTCCTACCAGACCC
AGACCAAgAGCCacAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAAgAGGGCTCTG
ACAGGCATTGCTGTGGAACAGGACAAGAACACCCAGGAGGTGTTTGCCCAGGTGAAGCAGATTTACAA
GACACCTCCAATCAAGtACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAACGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAGCTCC
AAGATGTGGTGAACCACAATGCCCAGGCTCTGAACACCCTGGTGAAGCAACTTTCCAGCAAgTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTTGAggatccgagctcggtaccaagcttaagtttaa
accgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
acaatagcaggcatgctggggatgcggtgggctctatggcttctgaggcggaaagaaccagctggggc
tctagggggtatccccacgcgccctgtagcggcgcattaagcgcggcgggtgtggtggttacgcgcag
cgtgaccgctacacttgccagcgccctagcgcccgctcctttcgctttcttcccttcctttctcgcca
cgttcgccggctttccccgtcaagctctaaatcgggggctccctttagggttccgatttagtgcttta
cggcacctcgaccccaaaaaacttgattagggtgatggttcacgtagtgggccatcgccctgatagac
ggtttttcgccctttgacgttggagtccacgttctttaatagtggactcttgttccaaactggaacaa
cactcaaccctatctcggtctattcttttgatttataagggattttgccgatttcggcctattggtta
aaaaatgagctgatttaacaaaaatttaacgcgaattaattctgtggaatgtgtgtcagttagggtgt
ggaaagtccccaggctccccagcaggcagaagtatgcaaagcatgcatctcaattagtcagcaaccag
gtgtggaaagtccccaggctccccagcaggcagaagtatgcaaagcatgcatctcaattagtcagcaa
ccatagtcccgcccctaactccgcccatcccgcccctaactccgcccagttccgcccattctccgccc
catggctgactaattttttttatttatgcagaggccgaggccgcctctgcctctgagctattccagaa
gtagtgaggaggcttttttggaggcctaggcttttgcaaaaagctcccgggagcttgtatatccattt
tcggatctgatcaagagacaggatgaggatcgtttcgcatgattgaacaagatggattgcacgcaggt
tctccggccgcttgggtggagaggctattcggctatgactgggcacaacagacaatcggctgctctga
tgccgccgtgttccggctgtcagcgcaggggcgcccggttctttttgtcaagaccgacctgtccggtg
ccctgaatgaactgcaggacgaggcagcgcggctatcgtggctggccacgacgggcgttccttgcgca
gctgtgctcgacgttgtcactgaagcgggaagggactggctgctattgggcgaagtgccggggcagga
tctcctgtcatctcaccttgctcctgccgagaaagtatccatcatggctgatgcaatgcggcggctgc
atacgcttgatccggctacctgcccattcgaccaccaagcgaaacatcgcatcgagcgagcacgtact
cggatggaagccggtcttgtcgatcaggatgatctggacgaagagcatcaggggctcgcgccagccga
actgttcgccaggctcaaggcgcgcatgcccgacggcgaggatctcgtcgtgacccatggcgatgcct
gcttgccgaatatcatggtggaaaatggccgcttttctggattcatcgactgtggccggctgggtgtg
gcggaccgctatcaggacatagcgttggctacccgtgatattgctgaagagcttggcggcgaatgggc
tgaccgcttcctcgtgctttacggtatcgccgctcccgattcgcagcgcatcgccttctatcgccttc
ttgacgagttcttctgagcgggactctggggttcgaaatgaccgaccaagcgacgcccaacctgccat
cacgagatttcgattccaccgccgccttctatgaaaggttgggcttcggaatcgttttccgggacgcc
ggctggatgatcctccagcgcggggatctcatgctggagttcttcgcccaccccaacttgtttattgc
agcttataatggttacaaataaagcaatagcatcacaaatttcacaaataaagcatttttttcactgc
attctagttgtggtttgtccaaactcatcaatgtatcttatcatgtctgtataccgtcgacctctagc
tagagcttggcgtaatcatggtcatagctgtttcctgtgtgaaattgttatccgctcacaattccaca
caacatacgagccggaagcataaagtgtaaagcctggggtgcctaatgagtgagctaactcacattaa
ttgcgttgcgctcactgcccgctttccagtcgggaaacctgtcgtgccagctgcattaatgaatcggc
caacgcgcggggagaggcggtttgcgtattgggcgctcttccgcttcctcgctcactgactcgctgcg
ctcggtcgttcggctgcggcgagcggtatcagctcactcaaaggcggtaatacggttatccacagaat
caggggataacgcaggaaagaacatgtgagcaaaaggccagcaaaaggccaggaaccgtaaaaaggcc
gcgttgctggcgtttttccataggctccgcccccctgacgagcatcacaaaaatcgacgctcaagtca
gaggtggcgaaacccgacaggactataaagataccaggcgtttccccctggaagctccctcgtgcgct
ctcctgttccgaccctgccgcttaccggatacctgtccgcctttctcccttcgggaagcgtggcgctt
tctcatagctcacgctgtaggtatctcagttcggtgtaggtcgttcgctccaagctgggctgtgtgca
cgaaccccccgttcagcccgaccgctgcgccttatccggtaactatcgtcttgagtccaacccggtaa
gacacgacttatcgccactggcagcagccactggtaacaggattagcagagcgaggtatgtaggcggt
gctacagagttcttgaagtggtggcctaactacggctacactagaagaacagtatttggtatctgcgc
tctgctgaagccagttaccttcggaaaaagagttggtagctcttgatccggcaaacaaaccaccgctg
gtagcggtggtttttttgtttgcaagcagcagattacgcgcagaaaaaaaggatctcaagaagatcct
ttgatcttttctacggggtctgacgctcagtggaacgaaaactcacgttaagggattttggtcatgag
attatcaaaaaggatcttcacctagatccttttaaattaaaaatgaagttttaaatcaatctaaagta
tatatgagtaaacttggtctgacagttaccaatgcttaatcagtgaggcacctatctcagcgatctgt
ctatttcgttcatccatagttgcctgactccccgtcgtgtagataactacgatacgggagggcttacc
atctggccccagtgctgcaatgataccgcgagacccacgctcaccggctccagatttatcagcaataa
accagccagccggaagggccgagcgcagaagtggtcctgcaactttatccgcctccatccagtctatt
aattgttgccgggaagctagagtaagtagttcgccagttaatagtttgcgcaacgttgttgccattgc
tacaggcatcgtggtgtcacgctcgtcgtttggtatggcttcattcagctccggttcccaacgatcaa
ggcgagttacatgatcccccatgttgtgcaaaaaagcggttagctccttcggtcctccgatcgttgtc
agaagtaagttggccgcagtgttatcactcatggttatggcagcactgcataattctcttactgtcat
gccatccgtaagatgcttttctgtgactggtgagtactcaaccaagtcattctgagaatagtgtatgc
ggcgaccgagttgctcttgcccggcgtcaatacgggataataccgcgccacatagcagaactttaaaa
gtgctcatcattggaaaacgttcttcggggcgaaaactctcaaggatcttaccgctgttgagatccag
ttcgatgtaacccactcgtgcacccaactgatcttcagcatcttttactttcaccagcgtttctgggt
gagcaaaaacaggaaggcaaaatgccgcaaaaaagggaataagggcgacacggaaatgttgaatactc
atactcttcctttttcaatattattgaagcatttatcagggttattgtctcatgagcggatacatatt
tgaatgtatttagaaaaataaacaaataggggttccgcgcacatttccccgaaaagtgccacctgacg
tc
Omicron Spike Del-19 (pZF47)
(SEQ ID NO: 57)
MFVFLVLLPLVSSQCVNLTTRTQLPPAYTNSFTRGVYYPDKVFRSSVLHSTQDLFLPFFSNVTWFHVI
SGTNGTKRFDNPVLPFNDGVYFASIEKSNIIRGWIFGTTLDSKTQSLLIVNNATNVVIKVCEFQFCND
PFLDHKNNKSWMESEFRVYSSANNCTFEYVSQPFLMDLEGKQGNFKNLREFVFKNIDGYFKIYSKHTP
IIVREPEDLPQGFSALEPLVDLPIGINITRFQTLLALHRSYLTPGDSSSGWTAGAAAYYVGYLQPRTF
LLKYNENGTITDAVDCALDPLSETKCTLKSFTVEKGIYQTSNFRVQPTESIVRFPNITNLCPFDEVFN
ATRFASVYAWNRKRISNCVADYSVLYNLAPFFTFKCYGVSPTKLNDLCFTNVYADSFVIRGDEVRQIA
PGQTGNIADYNYKLPDDFTGCVIAWNSNKLDSKVSGNYNYLYRLFRKSNLKPFERDISTEIYQAGNKP
CNGVAGFNCYFPLRSYSFRPTYGVGHQPYRVVVLSFELLHAPATVCGPKKSTNLVKNKCVNFNFNGLK
GTGVLTESNKKFLPFQQFGRDIADTTDAVRDPQTLEILDITPCSFGGVSVITPGTNTSNQVAVLYQGV
NCTEVPVAIHADQLTPTWRVYSTGSNVFQTRAGCLIGAEYVNNSYECDIPIGAGICASYQTQTKSHRR
ARSVASQSIIAYTMSLGAENSVAYSNNSIAIPTNFTISVTTEILPVSMTKTSVDCTMYICGDSTECSN
LLLQYGSFCTQLKRALTGIAVEQDKNTQEVFAQVKQIYKTPPIKYFGGFNFSQILPDPSKPSKRSFIE
DLLFNKVTLADAGFIKQYGDCLGDIAARDLICAQKFKGLTVLPPLLTDEMIAQYTSALLAGTITSGWT
FGAGAALQIPFAMQMAYRFNGIGVTQNVLYENQKLIANQFNSAIGKIQDSLSSTASALGKLQDVVNHN
AQALNTLVKQLSSKFGAISSVLNDIFSRLDKVEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICHDGKAHFPR
EGVFVSNGTHWFVTQRNFYEPQIITTDNTFVSGNCDVVIGIVNNTVYDPLQPELDSFKEELDKYFKNH
TSPDVDLGDISGINASVVNIQKEIDRLNEVAKNLNESLIDLQELGKYEQYIKWPWYIWLGFIAGLIAI
VMVTIMLCCMTSCCSCLKGCCSCGSCC*
Omicron BA.2.12.1 Spike Del-19 (pZF89)
(SEQ ID NO: 58)
MFVFLVLLPLVSSQCVNLITRTQSYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIKVCEFQFCNDP
FLDVYYHKNNKSWMESEFRVYSSANNCTFEYVSQPFLMDLEGKQGNFKNLREFVFKNIDGYFKIYSKH
TPINLGRDLPQGFSALEPLVDLPIGINITRFQTLLALHRSYLTPGDSSSGWTAGAAAYYVGYLQPRTF
LLKYNENGTITDAVDCALDPLSETKCTLKSFTVEKGIYQTSNFRVQPTESIVRFPNITNLCPFDEVFN
ATRFASVYAWNRKRISNCVADYSVLYNFAPFFAFKCYGVSPTKLNDLCFTNVYADSFVIRGNEVSQIA
PGQTGNIADYNYKLPDDFTGCVIAWNSNKLDSKVGGNYNYQYRLFRKSNLKPFERDISTEIYQAGNKP
CNGVAGFNCYFPLRSYGFRPTYGVGHQPYRVVVLSFELLHAPATVCGPKKSTNLVKNKCVNFNFNGLT
GTGVLTESNKKFLPFQQFGRDIADTTDAVRDPQTLEILDITPCSFGGVSVITPGTNTSNQVAVLYQGV
NCTEVPVAIHADQLTPTWRVYSTGSNVFQTRAGCLIGAEYVNNSYECDIPIGAGICASYQTQTKSHRR
ARSVASQSIIAYTMSLGAENLVAYSNNSIAIPTNFTISVTTEILPVSMTKTSVDCTMYICGDSTECSN
LLLQYGSFCTQLKRALTGIAVEQDKNTQEVFAQVKQIYKTPPIKYFGGFNFSQILPDPSKPSKRSFIE
DLLFNKVTLADAGFIKQYGDCLGDIAARDLICAQKFNGLTVLPPLLTDEMIAQYTSALLAGTITSGWT
FGAGAALQIPFAMQMAYRFNGIGVTQNVLYENQKLIANQFNSAIGKIQDSLSSTASALGKLQDVVNHN
AQALNTLVKQLSSKFGAISSVLNDILSRLDKVEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICHDGKAHFPR
EGVFVSNGTHWFVTQRNFYEPQIITTDNTFVSGNCDVVIGIVNNTVYDPLQPELDSFKEELDKYFKNH
TSPDVDLGDISGINASVVNIQKEIDRLNEVAKNLNESLIDLQELGKYEQYIKWPWYIWLGFIAGLIAI
VMVTIMLCCMTSCCSCLKGCCSCGSCC*
Omicron BA.4 Spike Del-19 (pZF90)
(SEQ ID NO: 59)
MFVFLVLLPLVSSQCVNLITRTQSYTNSFTRGVYYPDKVFRSSVLHSTQDLFLPFFSNVTWFHAISGT
NGTKRFDNPVLPFNDGVYFASTEKSNIIRGWIFGTTLDSKTQSLLIVNNATNVVIKVCEFQFCNDPFL
DVYYHKNNKSWMESEFRVYSSANNCTFEYVSQPFLMDLEGKQGNFKNLREFVFKNIDGYFKIYSKHTP
INLGRDLPQGFSALEPLVDLPIGINITRFQTLLALHRSYLTPGDSSSGWTAGAAAYYVGYLQPRTFLL
KYNENGTITDAVDCALDPLSETKCTLKSFTVEKGIYQTSNFRVQPTESIVRFPNITNLCPFDEVFNAT
RFASVYAWNRKRISNCVADYSVLYNFAPFFAFKCYGVSPTKLNDLCFTNVYADSFVIRGNEVSQIAPG
QTGNIADYNYKLPDDFTGCVIAWNSNKLDSKVGGNYNYRYRLFRKSNLKPFERDISTEIYQAGNKPCN
GVAGVNCYFPLQSYGFRPTYGVGHQPYRVVVLSFELLHAPATVCGPKKSTNLVKNKCVNFNFNGLTGT
GVLTESNKKFLPFQQFGRDIADTTDAVRDPQTLEILDITPCSFGGVSVITPGTNTSNQVAVLYQGVNC
TEVPVAIHADQLTPTWRVYSTGSNVFQTRAGCLIGAEYVNSSYECDIPIGAGICASYQTQTKSHRRAR
SVASQSIIAYTMSLGAENSVAYSNNSIAIPTNFTISVTTEILPVSMTKTSVDCTMYICGDSTECSNLL
LQYGSFCTQLKRALTGIAVEQDKNTQEVFAQVKQIYKTPPIKYFGGFNFSQILPDPSKPSKRSFIEDL
LFNKVTLADAGFIKQYGDCLGDIAARDLICAQKFNGLTVLPPLLTDEMIAQYTSALLAGTITSGWTFG
AGAALQIPFAMQMAYRFNGIGVTQNVLYENQKLIANQFNSAIGKIQDSLSSTASALGKLQDVVNHNAQ
ALNTLVKQLSSKFGAISSVLNDILSRLDKVEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAICHDGKAHFPREG
VFVSNGTHWFVTQRNFYEPQIITTDNTFVSGNCDVVIGIVNNTVYDPLQPELDSFKEELDKYFKNHTS
PDVDLGDISGINASVVNIQKEIDRLNEVAKNLNESLIDLQELGKYEQYIKWPWYIWLGFIAGLIAIVM
VTIMLCCMTSCCSCLKGCCSCGSCC*
Omicron BA.2 Spike Del-19 (pZF57)
(SEQ ID NO: 60)
MFVFLVLLPLVSSQCVNLITRTQSYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIKVCEFQFCNDP
FLDVYYHKNNKSWMESEFRVYSSANNCTFEYVSQPFLMDLEGKQGNFKNLREFVFKNIDGYFKIYSKH
TPINLGRDLPQGFSALEPLVDLPIGINITRFQTLLALHRSYLTPGDSSSGWTAGAAAYYVGYLQPRTF
LLKYNENGTITDAVDCALDPLSETKCTLKSFTVEKGIYQTSNFRVQPTESIVRFPNITNLCPFDEVFN
ATRFASVYAWNRKRISNCVADYSVLYNFAPFFAFKCYGVSPTKLNDLCFTNVYADSFVIRGNEVSQIA
PGQTGNIADYNYKLPDDFTGCVIAWNSNKLDSKVGGNYNYLYRLFRKSNLKPFERDISTEIYQAGNKP
CNGVAGFNCYFPLRSYGFRPTYGVGHQPYRVVVLSFELLHAPATVCGPKKSTNLVKNKCVNFNFNGLT
GTGVLTESNKKFLPFQQFGRDIADTTDAVRDPQTLEILDITPCSFGGVSVITPGTNTSNQVAVLYQGV
NCTEVPVAIHADQLTPTWRVYSTGSNVFQTRAGCLIGAEYVNNSYECDIPIGAGICASYQTQTKSHRR
ARSVASQSIIAYTMSLGAENSVAYSNNSIAIPTNFTISVTTEILPVSMTKTSVDCTMYICGDSTECSN
LLLQYGSFCTQLKRALTGIAVEQDKNTQEVFAQVKQIYKTPPIKYFGGFNFSQILPDPSKPSKRSFIE
DLLFNKVTLADAGFIKQYGDCLGDIAARDLICAQKFNGLTVLPPLLTDEMIAQYTSALLAGTITSGWT
FGAGAALQIPFAMQMAYRFNGIGVTQNVLYENQKLIANQFNSAIGKIQDSLSSTASALGKLQDVVNHN
AQALNTLVKQLSSKFGAISSVLNDILSRLDKVEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICHDGKAHFPR
EGVFVSNGTHWFVTQRNFYEPQIITTDNTFVSGNCDVVIGIVNNTVYDPLQPELDSFKEELDKYFKNH
TSPDVDLGDISGINASVVNIQKEIDRLNEVAKNLNESLIDLQELGKYEQYIKWPWYIWLGFIAGLIAI
VMVTIMLCCMTSCCSCLKGCCSCGSCC*

### Non-Limiting Comments

An Omicron-specific LNP-mRNA vaccine candidate was designed based on the full-length spike sequence of the Omicron variant (lineage B.1.1.529/BA.1) from two North America patients identified on Nov. 23, 2021 (GISAID EpiCoV: EPI_ISL_6826713 and EPI_ISL_6826714). The spike coding sequence of Wuhan-Hu-1 (WT) and Omicron variant were flanked by 5′ UTR, 3′ UTR and 3′ PolyA tail (FIG. 22A). Six proline mutations (HexaPro) were introduced into the spike gene sequence, as they were reported to improve spike protein stability and prefusion state. The furin cleave site (RRAR) in spike was replaced with GSAS stretch to keep integrity of S1 and S2 units. The transcribed spike mRNA was then encapsulated into lipid nanoparticles to produce WT and Omicron LNP-mRNAs, and characterized the quality and biophysical properties by downstream assays including dynamic light scattering, transmission electron microscope (TEM) and receptor binding assay.

The dynamic light scattering and transmission electron microscope were applied to evaluate the size distribution and shape of Omicron LNP-mRNA, which showed a monodispersed sphere shape with an average radius of 52 nm and polydispersity index of 0.17 (FIGS. 22C-22E). To evaluate the effectiveness of LNP-mRNA mediated Omicron spike expression in cells as well as the receptor binding ability of the designed Omicron HexaPro spike, Omicron LNP-mRNA was directly added to HEK293T cells 16 hours before subjecting cells to flow cytometry. Evident surface expression of functional Omicron HexaPro spike capable of binding to human angiotensin-converting enzyme-2 (hACE2) was observed by staining cells with hACE2-Fc fusion protein and PE anti-Fc secondary antibody (FIG. 22F). These data showed that the Omicron spike sequence was successfully encoded into an mRNA, encapsulated into the LNP, can be introduced into mammalian cells efficiently without additional manipulation, and express functional spike protein that binds to hACE2.

### Specific Binding and Neutralizing Antibody Response Elicited by Omicron LNP-mRNA Against the Omicron Variant

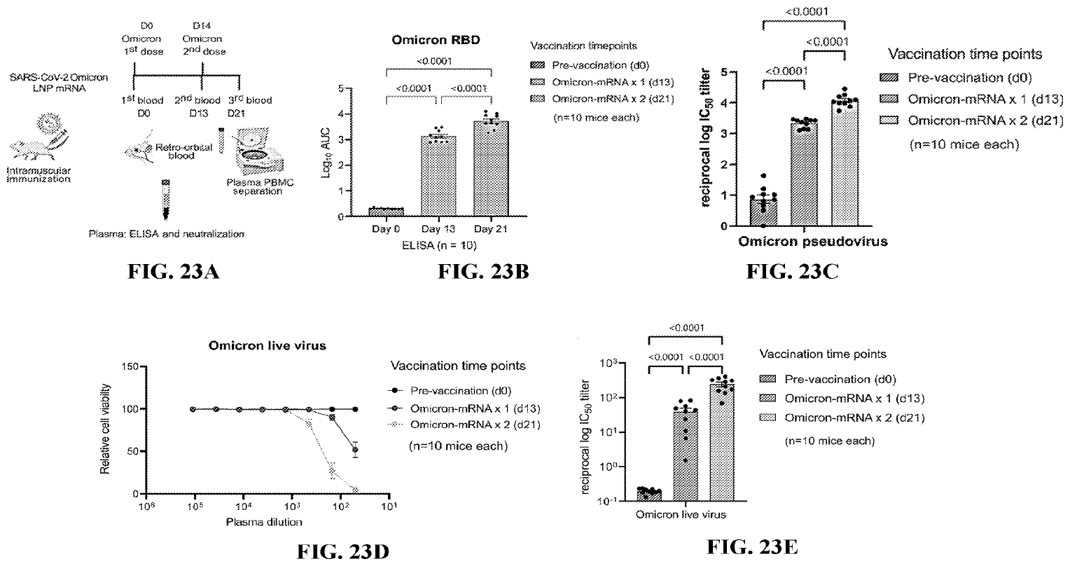

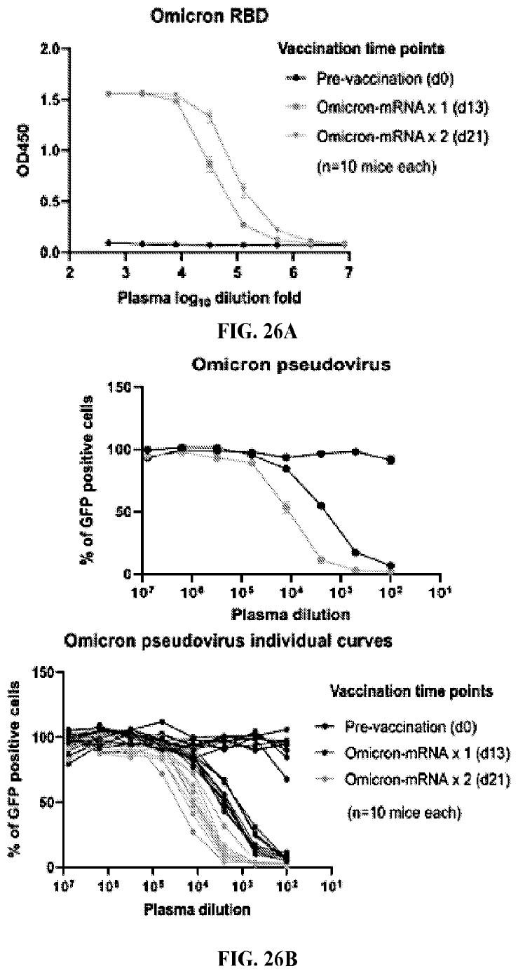

After ensuring functional spike expression mediated by Omicron LNP-mRNA, subsequent studies proceeded to characterize the immunogenicity of Omicron LNP-mRNA in vivo. In order to test rapid immune elicitation against Omicron variant, the following vaccination and testing schedule was performed. Two doses of 10 μg Omicron LNP-mRNA, as prime and boost two weeks apart were intramuscularly injected into ten C57BL/6Ncr (B6) mice (FIG. 23A; FIG. 26A). Retro-orbital blood was collected prior to immunization on day 0, 13 and 21, i.e. two weeks post prime (one day before boost), and one week post boost. Plasma from blood was then isolated, which was used in enzyme-linked immunosorbent assay (ELISA) and neutralization assay to quantify binding and neutralizing antibody titers. A significant increase in antibody titers against Omicron spike RBD was observed in ELISA and neutralization assays from plasma samples post prime and boost (FIGS. 23B-23C; FIG. 26A-26B). Neutralization with infectious virus (also commonly referred to as authentic virus or live virus) was performed using a local SARS-CoV-2 Omicron isolate in a biosafety level 3 (BSL3) setting (Methods), and validated that the plasma samples from mice vaccinated with Omicron-specific LNP-mRNA showed potent neutralization activity against infectious Omicron virus, with significant prime/boost effect (FIGS. 23D-23E). These data showed that the Omicron LNP-mRNA induced strong and specific antibody responses in vaccinated mice.

### Waning Immunity of WT LNP-mRNA Immunized Mice

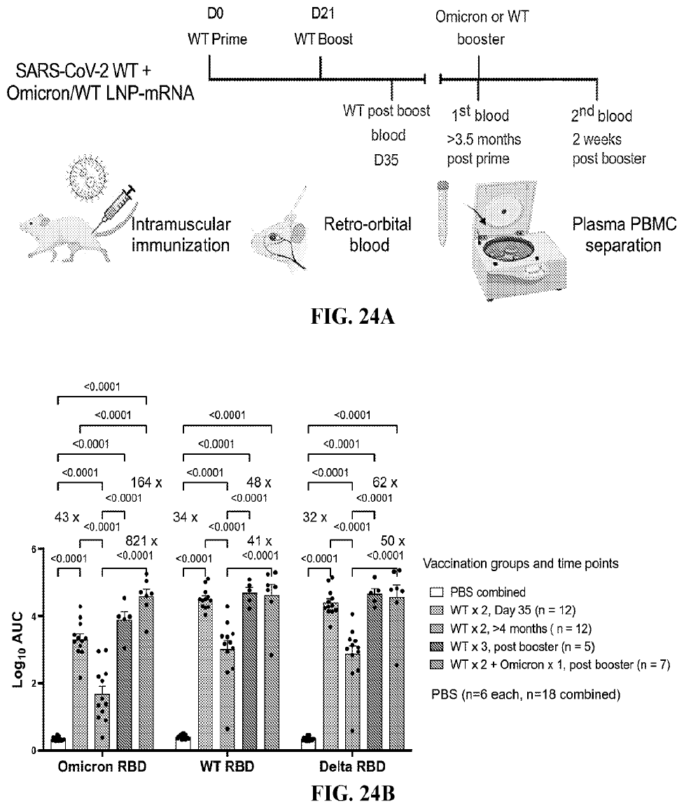

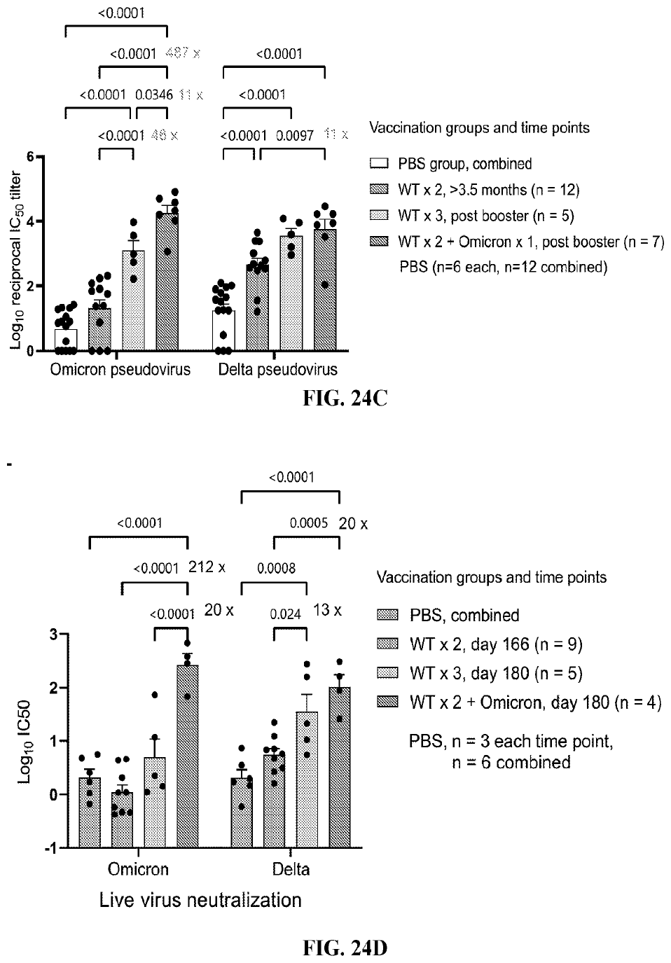

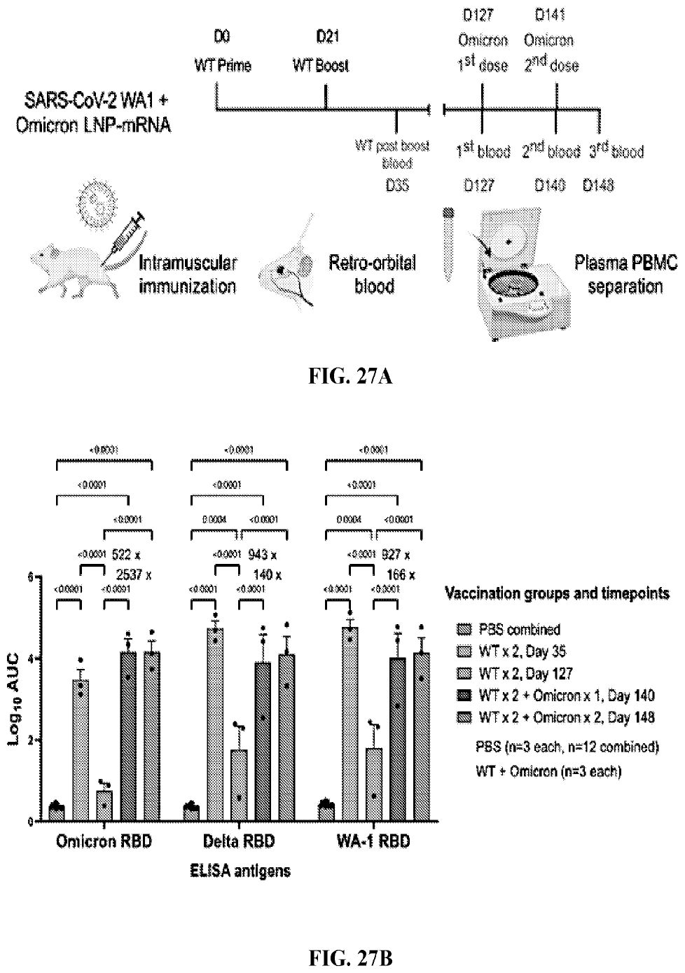

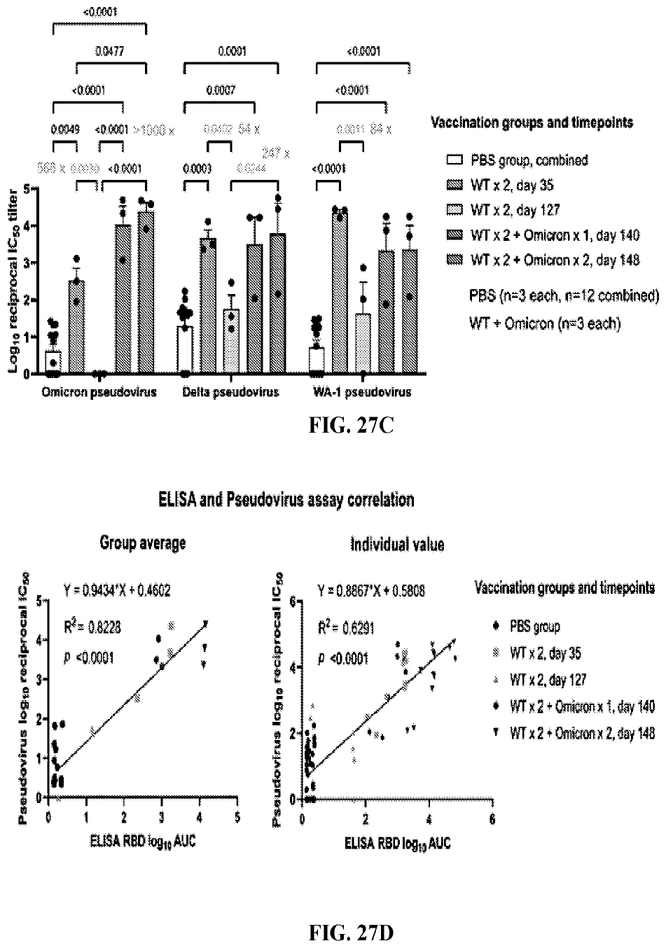

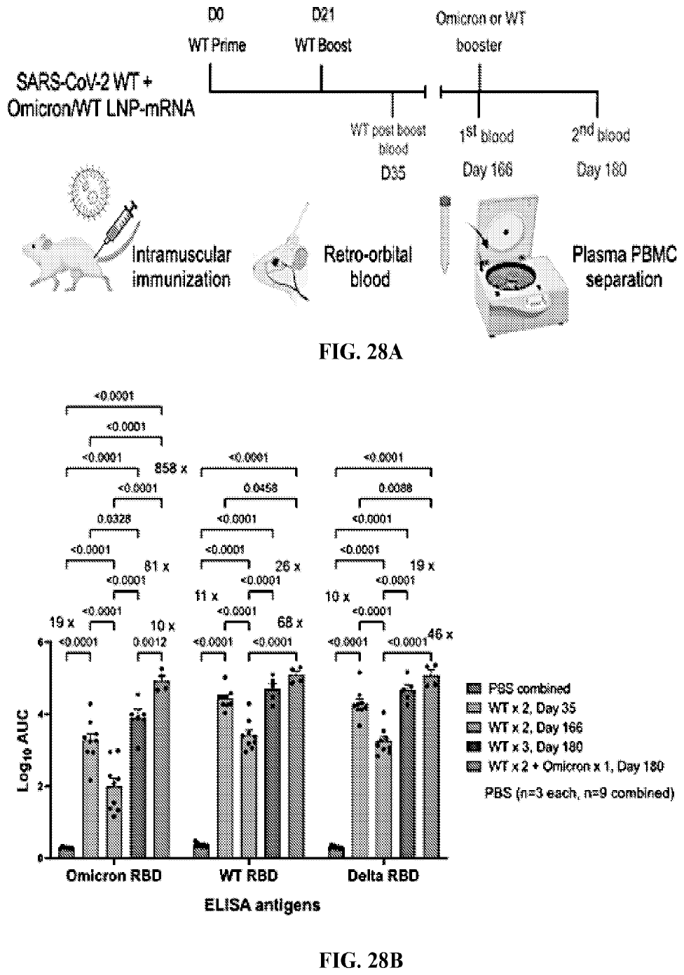

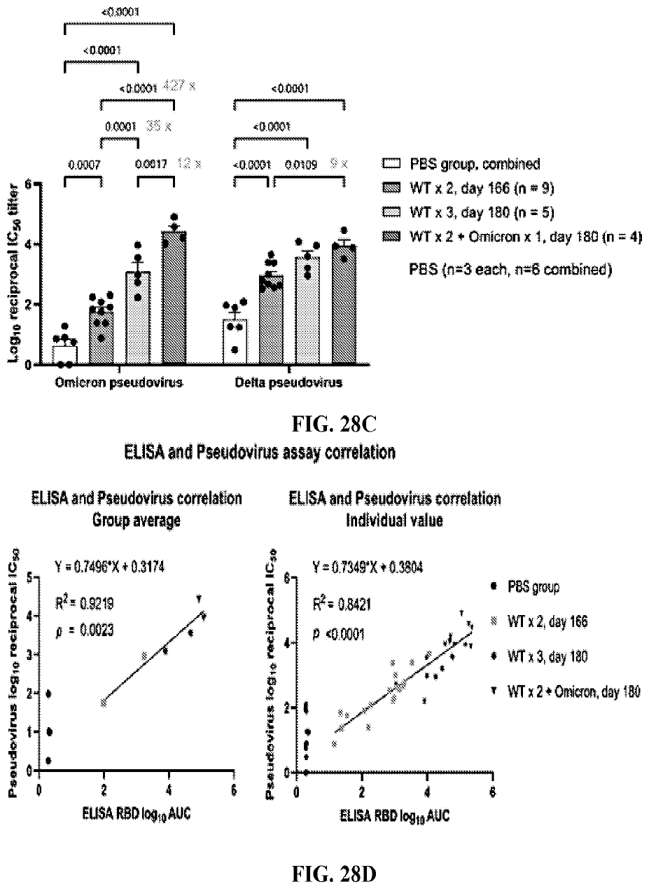

In light of the wide coverage of the ancestral WT-based LNP-mRNA vaccine (to model those widely administered in the current general population), subsequent studies sought to test: (i) the effect of WT LNP-mRNA vaccination against Omicron variant, (ii) the decay of immunity induced by WT LNP-mRNA over time, and (iii) whether a homologous WT LNP-mRNA booster or a heterologous Omicron LNP-mRNA booster could enhance the waning immunity against Omicron variant, WA-1 and/or Delta variant, and if there is a difference between homologous and heterologous boost. To gain initial answers to these questions in animal models, two cohorts of B6 mice were sequentially vaccinated with two doses of WT and one dose of WT or Omicron LNP-mRNA booster in two independent experiments (Batch 1 in FIG. 27 and batch 2 in FIG. 28). Over 100-day interval between 2nd dose of WT and WT/Omicron booster was ensured in order to observe the waning immunity in WT-vaccinated mice (the combined and individual datasets from the two independent experiments were presented in FIG. 24 and FIGS. 27-28 respectively). Blood samples of these animals was collected in a rational time series, including day 35 (2 weeks post 2^{nd} dose of WT LNP-mRNA), >3.5 months post 2^{nd} doses of WT LNP-mRNA (day 127 in batch 1 or day 166 in batch 2, immediately before WT/Omicron booster), ˜2 weeks post WT/Omicron LNP-mRNA booster (day 140, one day before the second Omicron booster in batch 1 or day 180 in batch 2), and day 148 (1 week post two doses of Omicron LNP-mRNA vaccination in batch 1).

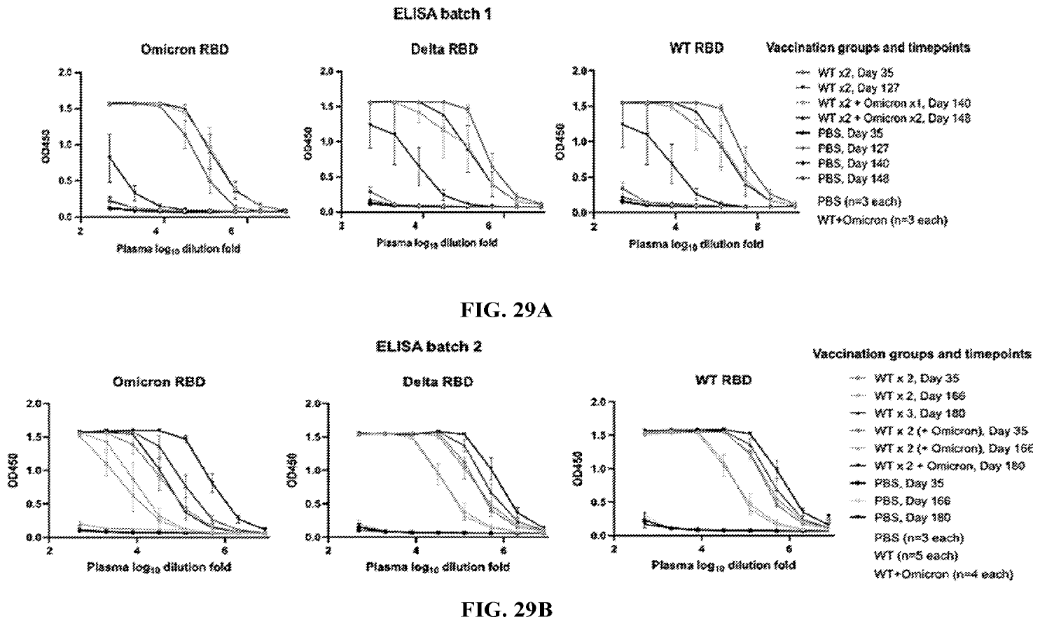

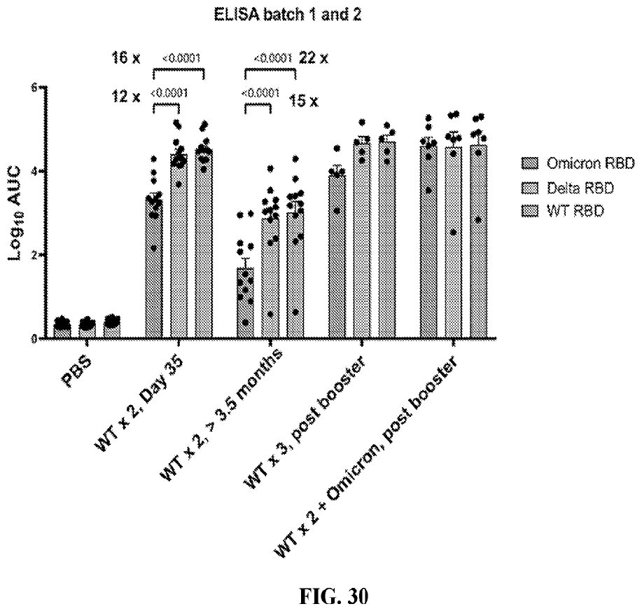

Plasma samples were isolated from blood samples and analyzed in ELISA and neutralization assays against SARS-CoV-2 Omicron, Delta or WA-1. Comparing to the titers against WA-1 and Delta RBD, the binding antibody titers against Omicron RBD elicited by WT mRNA-LNP were significantly weaker in samples from both day 35 and >3.5 months (FIG. 24B, FIG. 29-30). The group average Omicron reactivity is 15-fold (day 35) and 21-fold (>3.5 months) lower than that of WT RBD (fold change=ratio−1), and 11-fold (day 35) and 14-fold (>3.5 months) lower than Delta (FIG. 30). A steep (orders of magnitude) drop of antibody titers from mice immunized with WT LNP-mRNA was observed after three months (day 35 vs. >3.5 months) from all three RBD datasets. It is worth noting that the antibody titers >3.5 months post WT boost decreased to a level that is near-baseline (Phosphate buffered saline, PBS controls, FIG. 24), particularly for titers against Omicron RBD.

Heterologous Booster with Omicron LNP-mRNA as Compared to Homologous Booster with WT LNP-mRNA in Mice that Previously Received a Two-Dose WT LNP-mRNA Vaccination

A single dose booster shot, either a homologous booster with WT LNP-mRNA, or a heterologous booster with Omicron LNP-mRNA, drastically increased the antibody titers against Omicron RBD, by over 100-fold as compared to the sample right before booster shot (FIG. 24B), reaching a level comparable to the post-boost titer by Omicron LNP-mRNA alone (FIG. 23B). The mice that received the Omicron LNP-mRNA booster showed a trend of higher binding antibody titer against Omicron RBD than those administered with WT booster. Interestingly, the Omicron LNP-mRNA shot boosted not only titers against Omicron RBD, but also titers against Delta and WA-1 RBD, of which levels were comparable with those elicited by WT LNP-mRNA booster (FIG. 24B). For both WT and Omicron boosters, the extent of titer increase was more drastic in the Omicron RBD dataset than other RBD datasets, signifying the extra benefit of booster shots against Omicron variant (FIG. 24B). The antibody titers did not increase one week after a second booster of Omicron LNP-mRNA (FIG. 27B).

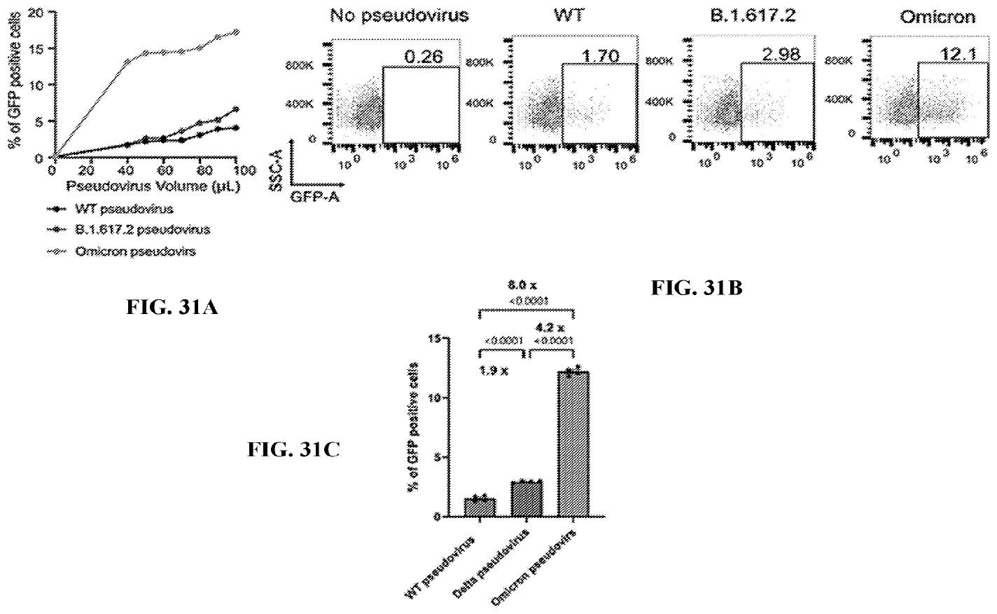

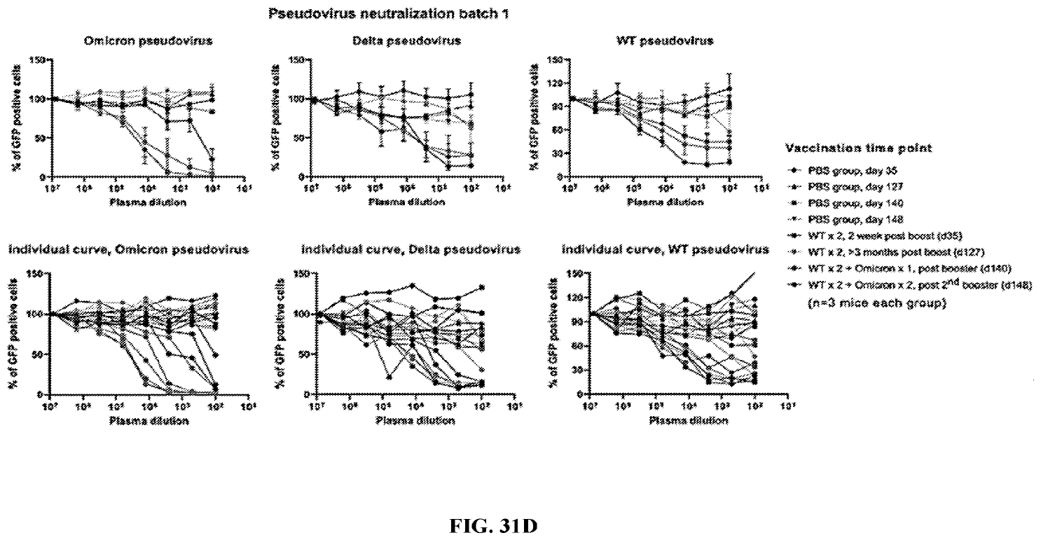

Because pseudovirus neutralization is a relatively safer and widely-used assay that strongly correlates with infectious virus results and has been regarded as a standard proxy by the field, subsequent studies then set out to first use pseudovirus neutralization assay to measure the neutralizing antibody responses induced by Omicron LNP-mRNA booster in these animals. We first generated human immunodeficiency virus-1 (HIV-1) based Omicron pseudovirus system, which contains identical Omicron mutations in vaccine antigen, but lacks the HexaPro or furin site modifications. Interestingly, it was found that under exactly the same virus production and assay conditions, the Omicron pseudovirus has higher infectivity than both WA-1 (8×increase) and Delta (4×) pseudoviruses (FIGS. 31A-C), which was also observed by another group, in concordance with the Omicron-hACE2 interactions from biophysical and structural studies, and correlated with higher transmissibility reported previously.

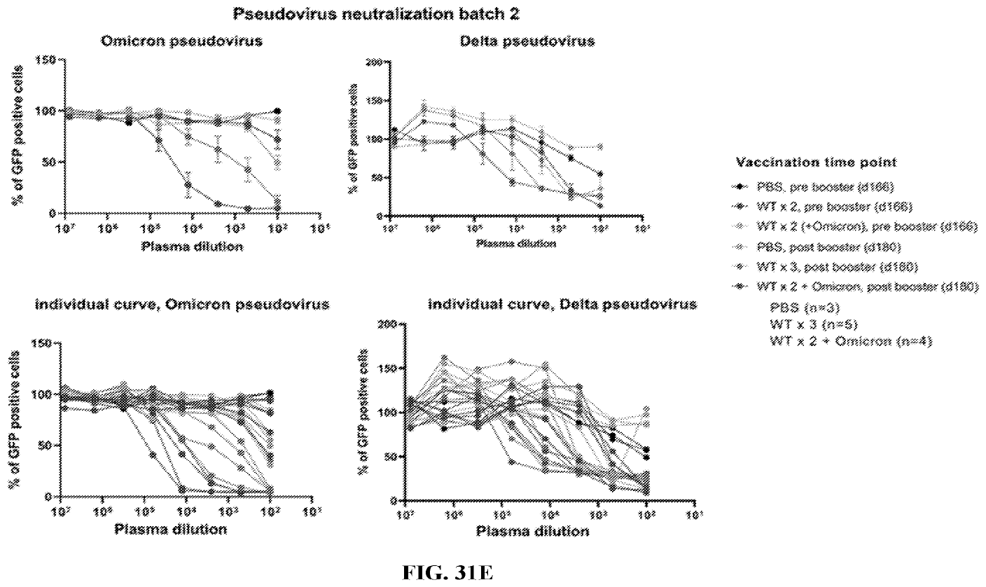

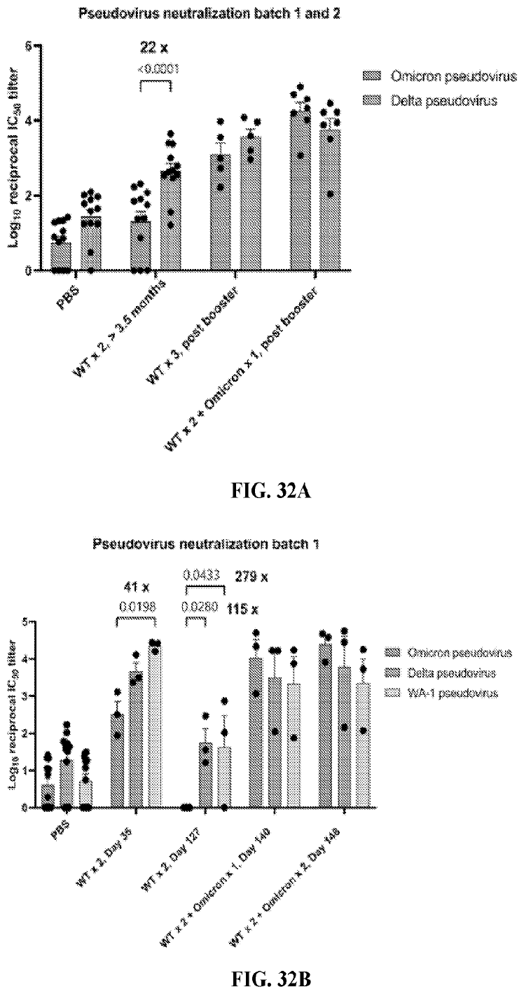

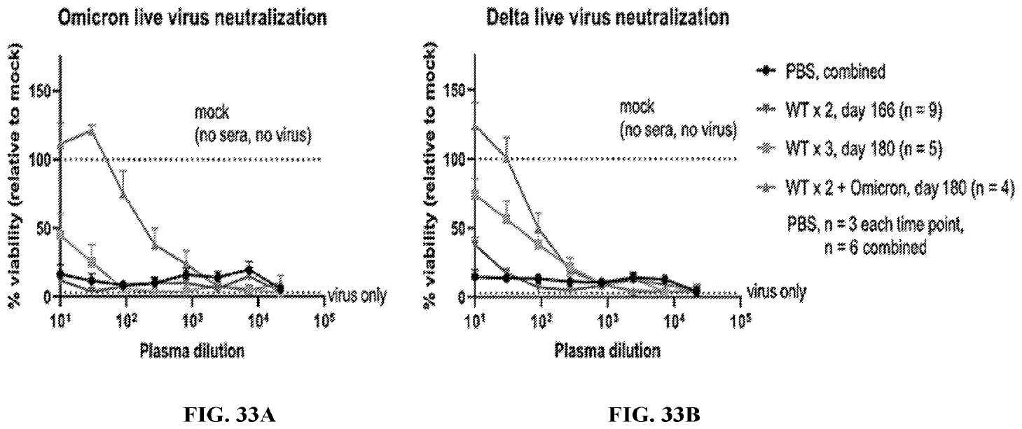

The pseudoviruses were then normalized by functional titers (number of infected cells/volume), and this system was used to perform pseudovirus neutralization assays on all of plasma samples collected (FIG. 31D-E). The neutralization results showed a consistent overall pattern as ELISA results, with a stronger contrast among titers against Omicron pseudovirus (FIG. 24C). On day 35 and >3.5 months post WT boost, the mice showed significantly lower neutralizing antibody titers against Omicron variant than titers against Delta variant or WA-1 (FIG. 32A-B). For the samples two weeks post boost (day 35), the group average Omicron neutralization reactivity is 40-fold lower than that of WA-1 RBD, and 10-fold lower than Delta (FIG. 32B). When comparing samples collected on day 35 and >3.5 months post WT boost, around two orders of magnitude (10s˜100s of fold change) time-dependent titer reduction was unequivocally observed in all three pseudovirus neutralization data (FIG. 23C). The Omicron-neutralization activity of WT vaccinated mice >3.5 months post boost was as low as PBS background (FIG. 23C). These data suggested that there was waning antibody immunity in the standard two-dose WT vaccinated animals, which lost neutralization ability against the Omicron variant pseudovirus.

A single booster shot of WT or Omicron LNP-mRNA vaccine enhanced the antibody titers against Omicron variant two weeks after the injection by >40-fold (FIG. 24C). The heterologous Omicron LNP-mRNA booster induced significantly higher neutralizing titer against Omicron pseudovirus than the homologous WT LNP-mRNA booster (FIG. 24c). The neutralizing titer after this surge by Omicron vaccine numerically surpassed the titer two weeks post WT vaccine boost (day 35, FIG. 27C). Interestingly, the Omicron mRNA vaccine also rescued the antibody titers against Delta and WA-1 pseudoviruses, with two orders of magnitude increase in both ELISA titers and neutralization activity (FIGS. 24B-24C). The neutralization titers of Delta pseudovirus were found similar between WT and Omicron booster groups (FIG. 24C). A second booster shot two weeks after the first of Omicron mRNA vaccine yielded little increase in neutralization activity against Omicron, WA-1 or Delta variants at the time measured (day 148, 1 week after the second dose) (FIG. 27C). Studies then further evaluated the effects of WT and Omicron LNP-mRNA boosters in infectious virus neutralization assay, which closely correlated with pseudovirus neutralization results. The Omicron LNP-mRNA booster led to over 200-fold increase in neutralizing titers of infectious Omicron virus (FIG. 24D), while WT booster induced a moderate increase (10-fold) in titers against Omicron live virus (FIG. 24D). A significant boost of infectious Delta virus neutralizing titers was observed in mice receiving WT (12-fold) and Omicron (19-fold) LNP-mRNA boosters. A 20-fold difference in post-booster (day 180) neutralizing titers against infectious Omicron virus was observed between WT and Omicron booster groups (FIG. 24D). Together, and without wishing to be bound by theory, these data suggest that while both WT LNP-mRNA and Omicron LNP-mRNA boosters can strengthen the waning immunity; however, the heterologous booster with Omicron-specific mRNA vaccination (WT×2+Omicron×1) has an effect significantly stronger than the homologous booster (WT×3) against the live virus of Omicron variant, with comparable activity against the Delta variant.

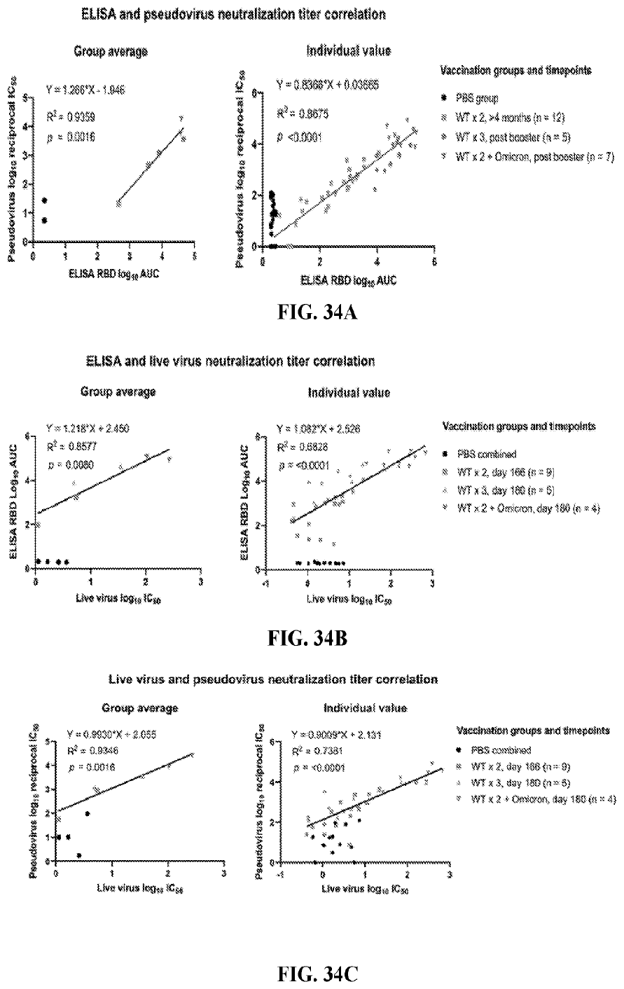

Overall, the ELISA titers, pseudovirus and infectious virus neutralization activity were significantly correlated with each other across all groups and animals tested (FIG. 34). These data suggested that a single dose of Omicron LNP-mRNA heterologous booster not only induced more potent anti-Omicron antibody response than WT booster, but also elicited broad activity against the WA-1 and Delta variant, in mouse models at the timepoints measured.

Cross Reactivity and Epitope Characterization of Plasma Antibodies from Homologous Omicron mRNA, WT mRNA or Heterologous WT+Omicron mRNA Vaccination Schemes

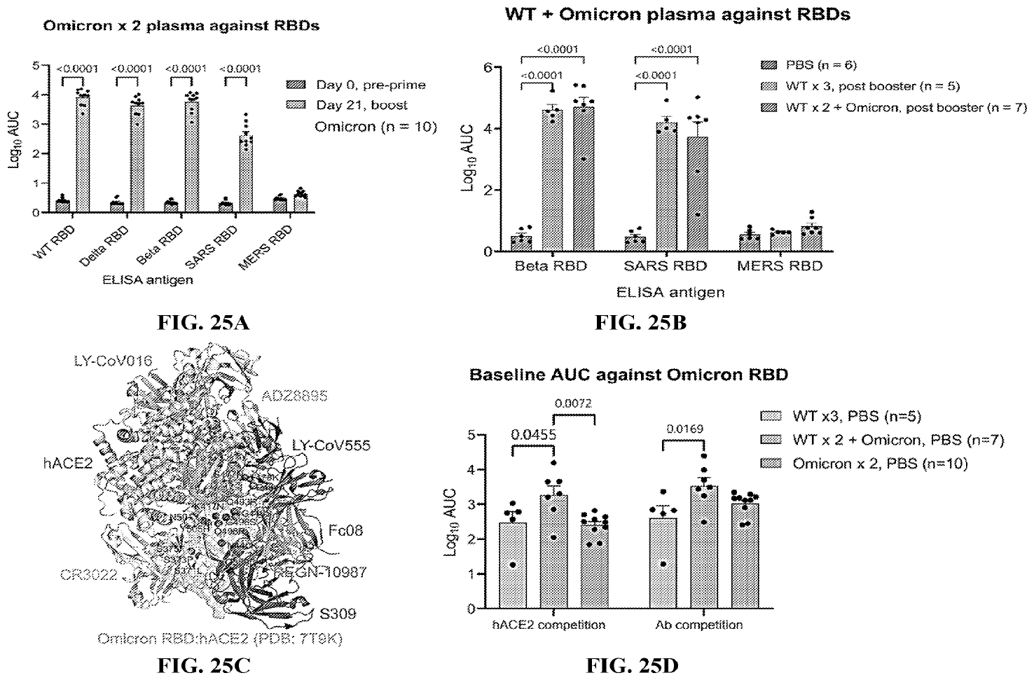

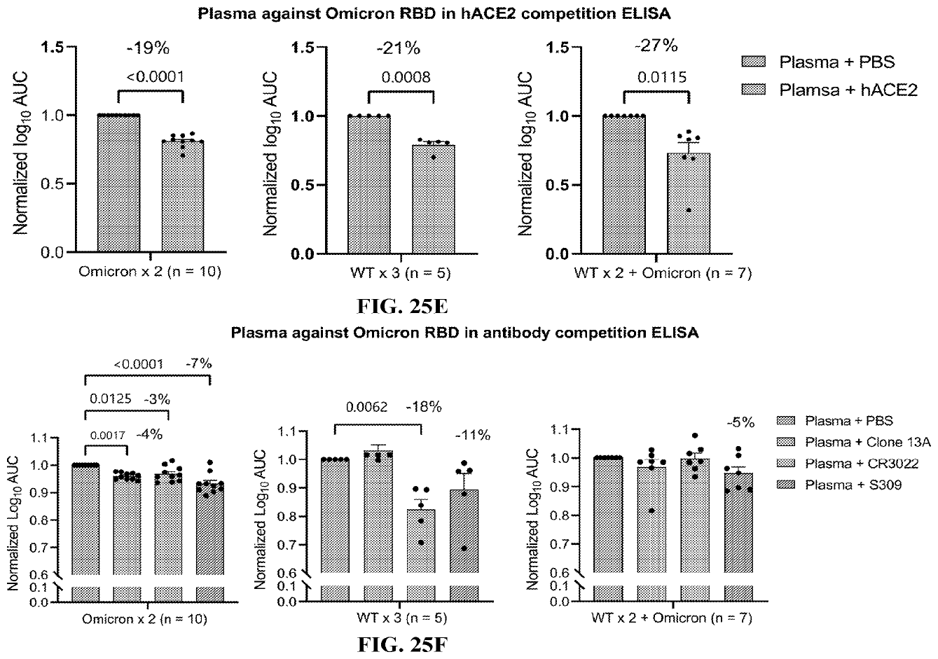

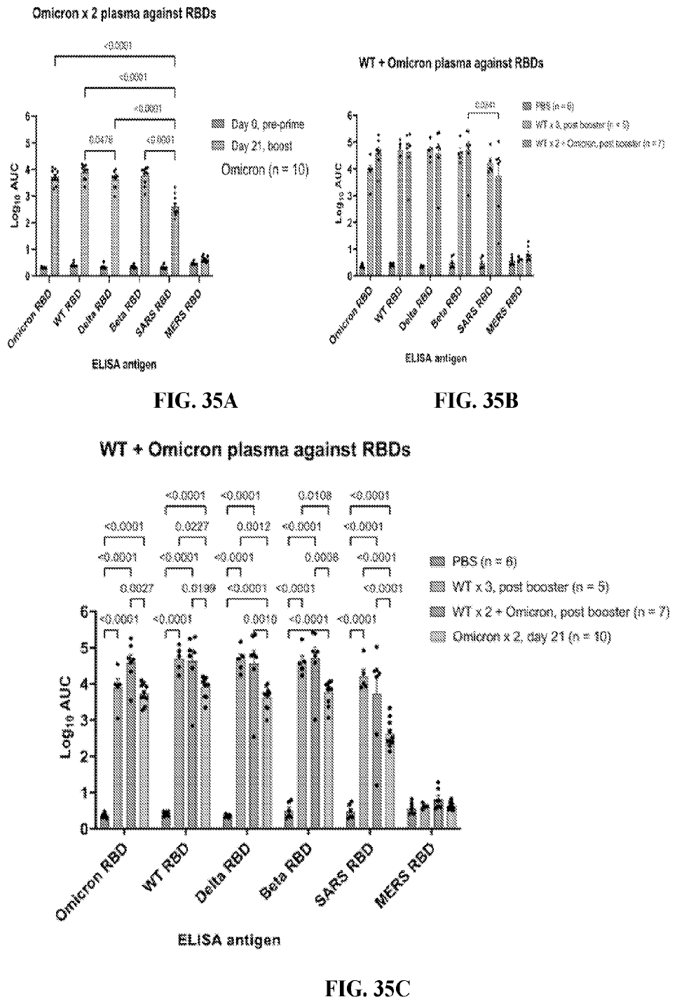

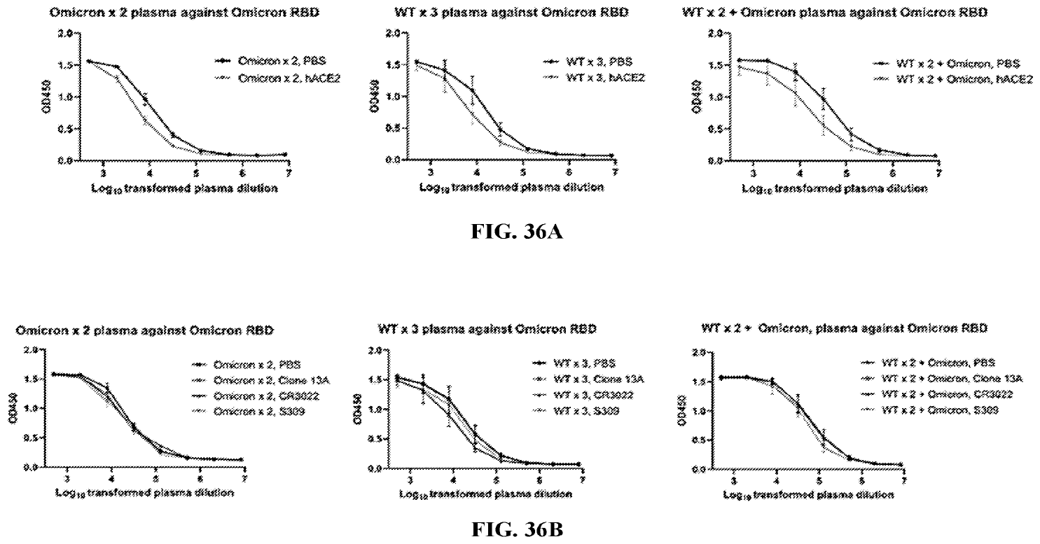

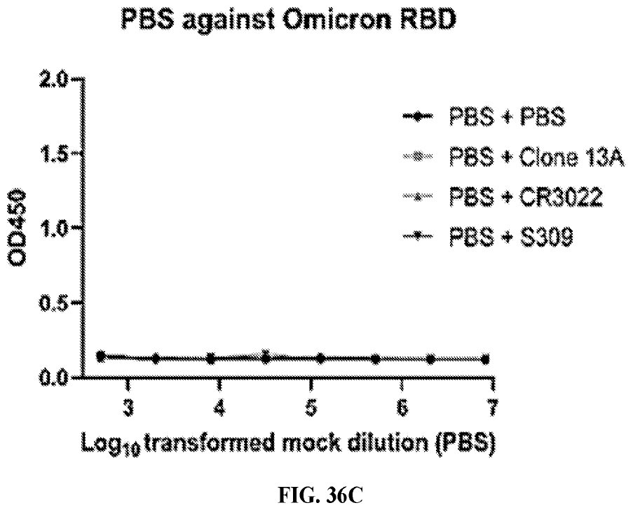

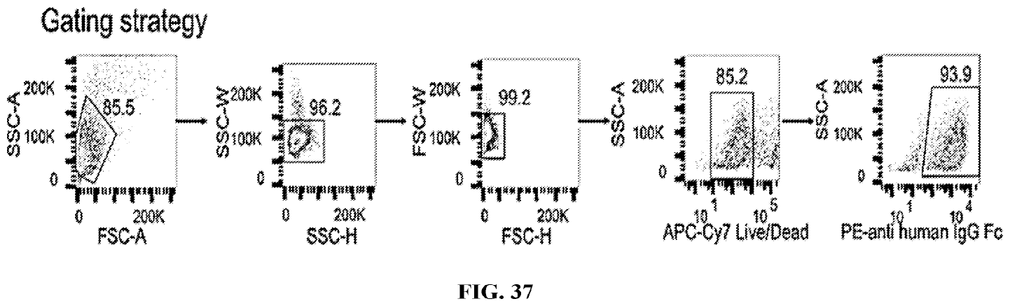

In light of the broad activity elicited by heterologous vaccination of WT and Omicron LNP-mRNA, subsequent studies then assessed if these vaccination schemes can induce antibody responses against other SARS-CoV-2 variants and other pathogenic Betacoronavirus species. It was sought to answer these questions by characterizing and comparing the anti-coronavirus cross reactivity conferred by Omicron mRNA vaccination alone, WT mRNA vaccination alone (homologous booster), or their uses in combination (Omicron mRNA vaccination as a heterologous booster on top of WT mRNA vaccination). The cross reactivity was evaluated using six spike RBDs, including SARS-CoV-2 WA-1, Beta (lineage B.1.351) variant, Delta variant, Omicron variant, SARS-CoV spike RBD (SARS RBD) and MERS-CoV spike RBD (MERS RBD). Two doses of Omicron LNP-mRNA induced high titers of antibodies that cross reacted with all spike RBDs tested except for MERS RBD, which shared low sequence identity (<40%) to SARS or SARS-CoV-2 spikes (FIG. 25A). The antibody titer against SARS RBD was significantly lower than those against SARS-CoV-2 WA-1 or variants (FIG. 35A). Among the SARS-CoV-2 variants characterized, the antibody response to Delta variant by Omicron LNP-mRNA was slightly weaker than others. Both WT and Omicron boosters after WT LNP-mRNA prime and boost led to potent antibody response to SARS-CoV and SARS-CoV-2 Beta variant (FIG. 25B), while the response to MERS RBD was negligible and similar to PBS control. Within each ELISA antigen except for MERS RBD and Omicron RBD, the antibody response post WT or Omicron boosters (3 shots total) was numerically higher than that of plasma samples post a two-dose Omicron vaccine (Omicron×2) (FIG. 35C).

A number of studies have shown that antibodies whose epitopes overlap with hACE2-binding motif were largely escaped by RBD mutations in variants of concerns, while antibodies whose epitopes fall outside the hACE2-binding motif were rarer and often exhibit broad neutralizing activity to SARS-like Betacoroanviruses (Sarbecoviruses). Because of such correlation between antibody epitope and cross reactivity, competition ELISA was performed using hACE2 or antibodies with known epitopes as competing agents to evaluate the epitopes, population and affinity of plasma antibodies elicited by Omicron or WT LNP-mRNA. The epitopes of RBD can be categorized into several major classes based on cluster analysis of available neutralizing antibody-RBD complex structures. Representative antibodies in each major epitope classes were displayed by aligning them with the recently solved Omicron RBD: hACE2 complex structure (FIG. 25C). hACE2 and antibody competition ELISA was then performed using hACE2, Clone 13A, S309 and CR3022 as competing reagents to see if and to what extent group A-D class I-III, epitopes overlapped with hACE2) and group E-F (class IV, S309 and CR3022) antibodies were induced by these immunization schemes. Low-density Omicron RBD was coated in ELISA plate to ensure adequate competition between plasma antibodies and competing hACE2 or antibodies. In two independent experiments (hACE2 and antibody competition assays), the baseline titer of heterologous Omicron booster treated mice (WT×2+Omicron) in the absence of competing reagents was significantly higher than those of homologous WT booster treated mice (WT×3), or mice receiving Omicron vaccination alone (Omicron×2) (FIG. 25D). Addition of high concentration hACE2 (Methods) resulted in a significant reduction of plasma antibody titers in mice vaccinated with Omicron (Omicron×2), WT (WT×3) or WT+Omicron (WT×2+Omicron) LNP-mRNA (FIG. 25E). In the antibody competition assay, we used three antibodies with known RBD epitopes. Two of them (CR3022 and S309) are well-characterized representative antibodies from non-hACE2 competing classes. The Clone 13A is a humanized neutralizing antibody disclosed by a previous study and has an epitope that overlaps with the hACE2 binding motif. All three antibodies led to a significant decrease of plasma titers from Omicron vaccinated mice (Omicron×2), while only CR3022 and S309 mediated a titer reduction in WT booster group (WT×3) (FIG. 25F). The WT+Omicron heterologous vaccination group showed minimal titer changes to all three antibodies (FIG. 25F). These data suggested that a significant percentage of the pool of antibodies elicited by Omicron- or WT-vaccination shared binding epitopes with hACE2. In addition, antibody competition ELISA showed that both Omicron LNP-mRNA and WT LNP-mRNA vaccinated animals contained plasma antibodies targeting rare epitopes in class IV (or group E/F), which often exhibit broad activity against Sarbecoviruses.

### Selected Discussion

The rapid spread of Omicron around the world, especially in countries with wide coverage of vaccines designed based on the ancestral antigen (e.g. WT mRNA vaccine), is particularly concerning. The extensive mutations in the Omicron spike gene mark a dramatic alteration in its antigenicity. Omicron has high transmissibility and high level of immune evasion from WT mRNA vaccine induced immunity, which was reported from various emerging literature. Omicron's strong association with reinfection or breakthrough infection and its heavily altered antigenicity prompted the idea of developing Omicron-specific mRNA vaccine.

As of Feb. 20, 2022, 4.35 billion people, i.e. 56% of the global population, received COVID-19 vaccination (Our World in Data). Almost all those vaccines were designed based on the antigen from the ancestral virus, including the two approved mRNA vaccine BNT162b2 and mRNA-1273. Individuals receiving existing COVID-19 vaccines have waning immunity over time. Consistent with past reports, the studies of the present disclosure observed a dramatic time-dependent decrease (around 40-fold) of antibody titers against Omicron, Delta variants and WA-1 strains 3 months after the second dose of WT mRNA vaccine in mice. This observed waning immunity is particularly concerning in the scenario of rapid spreading of Omicron variant, which largely escapes the humoral immune response elicited by WT mRNA vaccines as evident in published studies as well as in the current data. A recent report showed waning immunity in vaccinated individuals and that a booster shot using the WT based mRNA vaccine helps recover partial immunity. The data of the present disclosure showed that the neutralizing antibody titers after the boost with a WT based vaccine were still lower against Omicron than against WA-1 and other variants, urging for development and testing of an Omicron-specific vaccine. Vaccinee receiving heterologous vaccination of WT and Omicron LNP-mRNA have been exposed to both antigens and may have robust antibody response against cognate strains and other VoCs. Thus, it is important to evaluate and compare the immunogenicity of Omicron-specific vaccine candidate with WT vaccine as booster shots on top of two doses of WT mRNA vaccine. In fact, very recently, both Pfizer and Moderna have started their clinical trials to evaluate the efficacy of Omicron-specific mRNA vaccine in either homologous or heterologous vaccination settings. Moderna has released an updated Phase 2/3 clinical trial for their Omicron-specific mRNA vaccine (mRNA-1273.529) along with the WT vaccine mRNA-1273 against COVID-19 Omicron variant (NCT05249829). The scale and swiftness of initiating these clinical trials exemplify the clinical importance and urgent need of curbing the Omicron surge and evaluating the Omicron-specific mRNA vaccine.

In this study, a HexaPro-version of the full-length Omicron spike LNP-mRNA vaccine candidate was generated. In mouse models, it was found that it can induce potent Omicron-specific and broad anti-Sarbecovirus antibody response. With this vaccine candidate, its boosting effect was compared with WT counterpart on animals that previously received two-dose WT mRNA vaccine. An observation is that a single dose of WT or Omicron boosters significantly strengthened the waning immunity against Omicron and Delta variants. A number of recent preprints generated and tested Omicron-specific vaccine candidates, which had different vaccine antigen designs, compositions, and showed varying results of antibody responses alone or as boosters. Three of them focused on evaluation of Omicron RBD mRNA vaccine alone in mice through neutralization assay and reported antibody response against Omicron but not other variants. Two studies characterized the Omicron full-length spike mRNA stabilized by two proline mutations (S-2P) and compared their boosting efficacy with WT vaccine in mice and macaque. Recently published reports have shown that both WT and Omicron full-length spike mRNA boosters provided equivalent protection from Omicron challenge in non-human primates (NHPs) or mice. These results shared some commonalities, i.e. the effectiveness of an Omicron-specific vaccine; however, they diverged in the specific titers, as well as in the difference between WT- and Omicron-specific vaccines, potentially due to differences in vaccine antigen designs, compositions, modifications, experimental settings, animal models, or a combination of factors. The present study evaluated the potency of an Omicron-specific full-length spike mRNA vaccine with HexaPro mutations, which were shown to stabilize the spike in prefusion state. Through well-correlated data from ELISA, pseudovirus and infection virus neutralization assays, we showed that both WT and Omicron boosters significantly restored waning immunity against Omicron and Delta variants. Interestingly, without sacrificing potency against Delta, heterologous Omicron booster achieved significantly higher neutralizing titers against Omicron than homologous WT booster. This observation is in line with findings from heterologous booster vaccination of different COVID-19 vaccines in clinical trials. The broad anti-coronavirus activity after homologous or heterologous boosting was likely associated with plasma antibodies in rarer epitope classes, as observed in competition ELISA.

The neutralizing antibody level is highly predictive of immune protection from SARS-CoV-2 infection and the initial neutralization level is associated with decay of vaccine efficacy over time. Compared to WT booster, the studies presented herein found that Omicron booster group consistently showed 10-20 fold higher titers against Omicron variant in ELISA, pseudovirus and infectious virus neutralization assays. Within the WT vaccinated group, the titer contrast against Omicron vs. Delta variants persisted over time. Omicron-booster group have been exposed to both WT and Omicron antigens and showed equally potent titers against Omicron and Delta. While our study is in animals, the antibody responses to vaccination are conserved between mouse and human, highlighted by the fact that mice are the main preclinical model used by vaccine developers.

The titer against Omicron by single dose Omicron LNP-mRNA was similar to that observed 2 weeks post boost of WT LNP-mRNA (log 10 AUC or log 10 IC50 around 3), although it is still unclear whether the potency of the Omicron mRNA vaccine is associated with the high number of Omicron mutations. As various extent of cross reactivity was observed among WT and/or Omicron vaccinated animals, we sought to understand their cross-reactive immunity by characterizing vaccine-elicited antibody epitopes and population through competition ELISA. In the Omicron RBD competition ELISA, the baseline titer of Omicron LNP-mRNA booster group (WT×2+Omicron) was significantly higher than WT booster (WT×3) or Omicron LNP-mRNA (Omicron×2), which may explain its lower susceptibility to the block of competing antibodies. All three vaccination groups showed significant titer reduction in presence of hACE2, suggestive of abundant plasma antibody population sharing hACE2 binding epitopes, which are often associated with immune escape by variants mutations. The plasma from mice vaccinated with two doses of Omicron LNP-mRNA (Omicron×2) or three doses of WT LNP-mRNA (WT×3) exhibited comparable baseline titers and significant titer decrease when co-incubated with CR3022 or S309 blocking antibodies, indicating the existence of plasma antibody population sharing group E/F or class IV epitopes. Because of their similar baseline titers, the greater titer reduction in WT booster group may stem from larger population of group E/F antibodies, which was associated higher cross-reactive response against SARS RBD (FIG. 35C). Albeit insignificant, the titer change of Omicron booster group (WT×2+Omicron) by S309 antibody was greatest among three competing antibodies, hinting a role of epitope IV antibodies in the cross immunity elicited by heterologous vaccination of WT and Omicron LNP-mRNA.

In summary, this study generated an Omicron-specific HexaPro spike LNP-mRNA vaccine candidate, studied its immunogenicity, and compared it with the WT counterpart in the context of previously WT vaccinated animals. The results presented herein showed that a single dose of either a homologous booster with WT LNP-mRNA or a heterologous booster with Omicron LNP-mRNA restored the waning antibody response, with over 200-fold titer increase by Omicron boosters. Interestingly, the heterologous Omicron LNP-mRNA booster elicited Omicron neutralizing titers higher than the homologous WT booster. The heterologous Omicron booster shot provided strong neutralizing antibody response against Omicron variant and comparable humoral antibody against WA-1 and Delta variants. All three types of vaccination, including Omicron mRNA alone, WT mRNA alone, and Omicron as a heterologous booster on top of WT mRNA, elicited broad antibody responses, including activities against SARS-CoV-2 VoCs, as well as other Betacoronavirus species such as SARS-CoV, but not MERS-CoV. Together, these data provided direct proof-of-concept assessments of Omicron-specific mRNA vaccination in vivo, both alone and as a heterologous booster to the existing widely-used mRNA vaccine form.

### Example 6: Development of a Bivalent mRNA Vaccine Booster Against Omicron Subvariants BA.2, BA.2.12.1 and BA.5

### Materials and Methods

Molecular cloning and mRNA preparation. The WT and Delta spike plasmids were cloned in a previous study (Peng L, et al. Cell Rep Med (2022) 3:100634; Peng L, et al. bioRxiv (2022) Posted on 2022 Mar. 23). BA.2 spike plasmid was cloned based on the isolate sequencing data in GISAID EpiCoV (EPI_ISL_6795834.2) 9. WT, Delta and BA.2 spike plasmids were linearized by restriction enzymes and transcribed to mRNA by in vitro T7 RNA polymerase (NEB, Cat #E2060S) as previously described.

Cell culture. hACE2-293 FT and 293T cells were cultured in Dulbecco's minimal essential medium (DMEM, Fisher) supplemented with 10% fetal bovine serum (Hyclone) and penicillin (100 U/ml)-streptomycin (100 μg/ml). Cells were split every other day at a 1:4 ratio when confluency is over 90%.

Lipid nanoparticle mRNA preparation. In brief, lipids mixture was solubilized in ethanol and mixed with spike mRNA in pH 5.2 sodium acetate buffer. The mRNA encapsulated by LNP (LNP-mRNA) was then buffer exchanged to PBS using 100 kDa Amicon filter (Macrosep Centrifugal Devices 100K, 89131-992). The size distribution of LNP-mRNA was evaluated by dynamic light scatter (DynaPro NanoStar, Wyatt, WDPN-06). The Quant-iT™ RiboGreen™ (Thermo Fisher) RNA Assay was applied to determine encapsulation rate and mRNA amount.

Animal vaccination. Animal immunization was performed on 16-18 weeks female C57BL/6Ncr mice purchased from Charles River. Mice were vaccinated with two doses of 1.5 μg WT LNP-mRNA on day 0 and day 14 followed by 1.5 μg WT, Delta, Omicron BA.2 monovalent booster or Delta & BA.2 bivalent booster on day 29. The plasma samples were isolated from blood, which was collected before vaccination on day 0, two weeks after WT boost on day 28 and two weeks after monovalent or bivalent boosters on day 42.

ELISA and Neutralization assay. The binding and neutralizing antibody titers were determined by ELISA and pseudovirus neutralization assay as previously described. NanoGlo luciferase assay system (Promega N1120) was applied to determine the pseudovirus infection level in hACE2-293 FT cells. The ELISA antigens including RBDs of WT (Sino 40592-V08B), Delta (Sino 40592-V08H90), Omicron BA.2 (Acro SPD-C522g-100 μg), BA.2.12.1 (Acro SPD-C522q-100 μg) and BA.4/5 (Acro SPD-C522r-100 μg) were purchased from Sino Biological and AcroBiosystems. The ELISA ECD antigens including WT (Sino 40589-V08B1), Delta (Sino 40589-V08B16), Omicron BA.2 (Acro SPN-C5223-50 μg), BA.2.12.1 (Acro SPN-C522d-50 μg) and BA.4/5 (SPN-C5229-50 μg) were purchased from Sino Biological and AcroBiosystems. The pseudovirus plasmids of spike without HexaPro mutations were generated based on the WT plasmid which was a gift from Dr. Bieniasz's lab.

pZF89 Omicron BA.2.12.1 Spike Pseudovirus del19
(SEQ ID NO: 36)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccATgTTTGTGTTCCTGGTGCTGCTGCCACTGGTGTCCAGCCAGTGTGTG
AACCTGATCACCAGGACCCAAAGCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCACGTGTCTGGCACCAATGGCACCAAGAGGTTTGACAACCCTGTGCTGCCATTCAAT
GATGGAGTCTACTTTGCCAGCACCGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGacGTGTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGGCAGAGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGacGAGGTGTTCAAcGCCACCAGGTTTGCCTCTGTCTATGCCTGGAACAGGAAGAGGATTAGC
AACTGTGTGGCTGACTACTCTGTGCTCTACAACTTCGCCCCCTTCttCGCCTTCAAGTGTTATGGAGT
GAGCCCAACCAAACTGAATGACCTGTGTTTCACCAATGTCTATGCTGACTCCTTTGTGATTAGGGGAA
ACGAGGTGAGCCAGATTGCCCCTGGACAAACAGGCAACATTGCTGACTACAACTACAAACTGCCTGAT
GACTTCACAGGCTGTGTGATTGCCTGGAACAGCAACAAqCTGGACAGCAAGGTGGGCGGCAACTACAA
CTACCAGTACAGACTGTTCAGGAAGAGCAACCTGAAACCATTTGAGAGGGACATCAGCACAGAGATTT
ACCAGGCTGGCAaCAagCCATGTAATGGAGTGGCCGGCTTCAACTGTTACTTTCCACTCCggTCCTAT
GGCTTCCggCCAACCtacGGAGTGGGCcACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACCGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGqCGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GtacGTGAACAACTCCTATGAGTGTGACATCCCAATTGGAGCAGGCATCTGTGCCTCCTACCAGACCC
AGACCAAgAGCCacAGGAGGGCAAGGTCTGTGGCAAGCCAGAGCATCATTGCCTACACAATGAGTCTG
GGAGCAGAGAACCTGGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAAgAGGGCTCTG
ACAGGCATTGCTGTGGAACAGGACAAGAACACCCAGGAGGTGTTTGCCCAGGTGAAGCAGATTTACAA
GACACCTCCAATCAAGtACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAACGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAGCTCC
AAGATGTGGTGAACCACAATGCCCAGGCTCTGAACACCCTGGTGAAGCAACTTTCCAGCAAgTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTTGAggatccgagctcggtaccaagcttaagtttaa
accgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtggggtggggcaggacagcaagggggaggattgggaag
acaatagcaggcatgctggggatgcggtgggctctatggcttctgaggcggaaagaaccagctggggc
tctagggggtatccccacgcgccctgtagcggcgcattaagcgcggcgggtgtggtggttacgcgcag
cgtgaccgctacacttgccagcgccctagcgcccgctcctttcgctttcttcccttcctttctcgcca
cgttcgccggctttccccgtcaagctctaaatcgggggctccctttagggttccgatttagtgcttta
cggcacctcgaccccaaaaaacttgattagggtgatggttcacgtagtgggccatcgccctgatagac
ggtttttcgccctttgacgttggagtccacgttctttaatagtggactcttgttccaaactggaacaa
cactcaaccctatctcggtctattcttttgatttataagggattttgccgatttcggcctattggtta
aaaaatgagctgatttaacaaaaatttaacgcgaattaattctgtggaatgtgtgtcagttagggtgt
ggaaagtccccaggctccccagcaggcagaagtatgcaaagcatgcatctcaattagtcagcaaccag
gtgtggaaagtccccaggctccccagcaggcagaagtatgcaaagcatgcatctcaattagtcagcaa
ccatagtcccgcccctaactccgcccatcccgcccctaactccgcccagttccgcccattctccgccc
catggctgactaattttttttatttatgcagaggccgaggccgcctctgcctctgagctattccagaa
gtagtgaggaggcttttttggaggcctaggcttttgcaaaaagctcccgggagcttgtatatccattt
tcggatctgatcaagagacaggatgaggatcgtttcgcatgattgaacaagatggattgcacgcaggt
tctccggccgcttgggtggagaggctattcggctatgactgggcacaacagacaatcggctgctctga
tgccgccgtgttccggctgtcagcgcaggggcgcccggttctttttgtcaagaccgacctgtccggtg
ccctgaatgaactgcaggacgaggcagcgcggctatcgtggctggccacgacgggcgttccttgcgca
gctgtgctcgacgttgtcactgaagcgggaagggactggctgctattgggcgaagtgccggggcagga
tctcctgtcatctcaccttgctcctgccgagaaagtatccatcatggctgatgcaatgcggcggctgc
atacgcttgatccggctacctgcccattcgaccaccaagcgaaacatcgcatcgagcgagcacgtact
cggatggaagccggtcttgtcgatcaggatgatctggacgaagagcatcaggggctcgcgccagccga
actgttcgccaggctcaaggcgcgcatgcccgacggcgaggatctcgtcgtgacccatggcgatgcct
gcttgccgaatatcatggtggaaaatggccgcttttctggattcatcgactgtggccggctgggtgtg
gcggaccgctatcaggacatagcgttggctacccgtgatattgctgaagagcttggcggcgaatgggc
tgaccgcttcctcgtgctttacggtatcgccgctcccgattcgcagcgcatcgccttctatcgccttc
ttgacgagttcttctgagcgggactctggggttcgaaatgaccgaccaagcgacgcccaacctgccat
cacgagatttcgattccaccgccgccttctatgaaaggttgggcttcggaatcgttttccgggacgcc
ggctggatgatcctccagcgcggggatctcatgctggagttcttcgcccaccccaacttgtttattgc
agcttataatggttacaaataaagcaatagcatcacaaatttcacaaataaagcatttttttcactgc
attctagttgtggtttgtccaaactcatcaatgtatcttatcatgtctgtataccgtcgacctctagc
tagagcttggcgtaatcatggtcatagctgtttcctgtgtgaaattgttatccgctcacaattccaca
caacatacgagccggaagcataaagtgtaaagcctggggtgcctaatgagtgagctaactcacattaa
ttgcgttgcgctcactgcccgctttccagtcgggaaacctgtcgtgccagctgcattaatgaatcggc
caacgcgcggggagaggcggtttgcgtattgggcgctcttccgcttcctcgctcactgactcgctgcg
ctcggtcgttcggctgcggcgagcggtatcagctcactcaaaggcggtaatacggttatccacagaat
caggggataacgcaggaaagaacatgtgagcaaaaggccagcaaaaggccaggaaccgtaaaaaggcc
gcgttgctggcgtttttccataggctccgcccccctgacgagcatcacaaaaatcgacgctcaagtca
gaggtggcgaaacccgacaggactataaagataccaggcgtttccccctggaagctccctcgtgcgct
ctcctgttccgaccctgccgcttaccggatacctgtccgcctttctcccttcgggaagcgtggcgctt
tctcatagctcacgctgtaggtatctcagttcggtgtaggtcgttcgctccaagctgggctgtgtgca
cgaaccccccgttcagcccgaccgctgcgccttatccggtaactatcgtcttgagtccaacccggtaa
gacacgacttatcgccactggcagcagccactggtaacaggattagcagagcgaggtatgtaggcggt
gctacagagttcttgaagtggtggcctaactacggctacactagaagaacagtatttggtatctgcgc
tctgctgaagccagttaccttcggaaaaagagttggtagctcttgatccggcaaacaaaccaccgctg
gtagcggtggtttttttgtttgcaagcagcagattacgcgcagaaaaaaaggatctcaagaagatcct
ttgatcttttctacggggtctgacgctcagtggaacgaaaactcacgttaagggattttggtcatgag
attatcaaaaaggatcttcacctagatccttttaaattaaaaatgaagttttaaatcaatctaaagta
tatatgagtaaacttggtctgacagttaccaatgcttaatcagtgaggcacctatctcagcgatctgt
ctatttcgttcatccatagttgcctgactccccgtcgtgtagataactacgatacgggagggcttacc
atctggccccagtgctgcaatgataccgcgagacccacgctcaccggctccagatttatcagcaataa
accagccagccggaagggccgagcgcagaagtggtcctgcaactttatccgcctccatccagtctatt
aattgttgccgggaagctagagtaagtagttcgccagttaatagtttgcgcaacgttgttgccattgc
tacaggcatcgtggtgtcacgctcgtcgtttggtatggcttcattcagctccggttcccaacgatcaa
ggcgagttacatgatcccccatgttgtgcaaaaaagcggttagctccttcggtcctccgatcgttgtc
agaagtaagttggccgcagtgttatcactcatggttatggcagcactgcataattctcttactgtcat
gccatccgtaagatgcttttctgtgactggtgagtactcaaccaagtcattctgagaatagtgtatgc
ggcgaccgagttgctcttgcccggcgtcaatacgggataataccgcgccacatagcagaactttaaaa
gtgctcatcattggaaaacgttcttcggggcgaaaactctcaaggatcttaccgctgttgagatccag
ttcgatgtaacccactcgtgcacccaactgatcttcagcatcttttactttcaccagcgtttctgggt
gagcaaaaacaggaaggcaaaatgccgcaaaaaagggaataagggcgacacggaaatgttgaatactc
atactcttcctttttcaatattattgaagcatttatcagggttattgtctcatgagcggatacatatt
tgaatgtatttagaaaaataaacaaataggggttccgcgcacatttccccgaaaagtgccacctgacg
tc
pZF90 Omicron BA.4 Spike Pseudovirus del19
(SEQ ID NO: 37)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccATgTTTGTGTTCCTGGTGCTGCTGCCACTGGTGTCCAGCCAGTGTGTG
AACCTGATCACCAGGACCCAAAGCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCTCTGGCACCAATGGCACCAAGAGGTTTGACAACCCTGTGCTGCCATTCAATGATGGA
GTCTACTTTGCCAGCACCGAGAAGAGCAACATCATCAGGGGCTGGATTTTTGGCACCACCCTGGACAG
CAAGACCCAGTCCCTGCTGATTGTGAACAATGCCACCAATGTGGTGATTAAGGTGTGTGAGTTCCAGT
TCTGTAATGACCCATTCCTGGacGTGTACTACCACAAGAACAACAAGTCCTGGATGGAGTCTGAGTTC
AGGGTCTACTCCTCTGCCAACAACTGTACCTTTGAATATGTGAGCCAACCATTCCTGATGGACTTGGA
GGGCAAGCAGGGCAACTTCAAGAACCTGAGGGAGTTTGTGTTCAAGAACATTGATGGCTACTTCAAGA
TTTACAGCAAACACACACCAATCAACCTGGGCAGAGACCTGCCACAGGGCTTCTCTGCCTTGGAACCA
CTGGTGGACCTGCCAATTGGCATCAACATCACCAGGTTCCAGACCCTGCTGGCTCTGCACAGGTCCTA
CCTGACACCTGGAGACTCCTCCTCTGGCTGGACAGCAGGAGCAGCAGCCTACTATGTGGGCTACCTCC
AACCAAGGACCTTCCTGCTGAAATACAATGAGAATGGCACCATCACAGATGCTGTGGACTGTGCCCTG
GACCCACTGTCTGAGACCAAGTGTACCCTGAAATCCTTCACAGTGGAGAAGGGCATCTACCAGACCAG
CAACTTCAGGGTCCAACCAACAGAGAGCATTGTGAGGTTTCCAAACATCACCAACCTGTGTCCATTTG
acGAGGTGTTCAACGCCACCAGGTTTGCCTCTGTCTATGCCTGGAACAGGAAGAGGATTAGCAACTGT
GTGGCTGACTACTCTGTGCTCTACAACTTCGCCCCCTTCttCGCCTTCAAGTGTTATGGAGTGAGCCC
AACCAAACTGAATGACCTGTGTTTCACCAATGTCTATGCTGACTCCTTTGTGATTAGGGGAAACGAGG
TGAGCCAGATTGCCCCTGGACAAACAGGCAACATTGCTGACTACAACTACAAACTGCCTGATGACTTC
ACAGGCTGTGTGATTGCCTGGAACAGCAACAAqCTGGACAGCAAGGTGGGCGGCAACTACAACTACAG
ATACAGACTGTTCAGGAAGAGCAACCTGAAACCATTTGAGAGGGACATCAGCACAGAGATTTACCAGG
CTGGCAaCAagCCATGTAATGGAGTGGCCGGCGTGAACTGTTACTTTCCACTCCAgTCCTATGGCTTC
CggCCAACCtacGGAGTGGGCcACCAACCATACAGGGTGGTGGTGCTGTCCTTTGAACTGCTCCATGC
CCCTGCCACAGTGTGTGGACCAAAGAAGAGCACCAACCTGGTGAAGAACAAGTGTGTGAACTTCAACT
TCAATGGACTGACCGGCACAGGAGTGCTGACAGAGAGCAACAAGAAGTTCCTGCCATTCCAACAGTTT
GGCAGGGACATTGCTGACACCACAGATGCTGTGAGGGACCCACAGACCTTGGAGATTCTGGACATCAC
ACCATGTTCCTTTGGAGGAGTGTCTGTGATTACACCTGGCACCAACACCAGCAACCAGGTGGCTGTGC
TCTACCAGGgcGTGAACTGTACTGAGGTGCCTGTGGCTATCCATGCTGACCAACTTACACCAACCTGG
AGGGTCTACAGCACAGGCAGCAATGTGTTCCAGACCAGGGCTGGCTGTCTGATTGGAGCAGAGtacGT
GAACAGCTCCTATGAGTGTGACATCCCAATTGGAGCAGGCATCTGTGCCTCCTACCAGACCCAGACCA
AgAGCCacAGGAGGGCAAGGTCTGTGGCAAGCCAGAGCATCATTGCCTACACAATGAGTCTGGGAGCA
GAGAACTCTGTGGCTTACAGCAACAACAGCATTGCCATCCCAACCAACTTCACCATCTCTGTGACCAC
AGAGATTCTGCCTGTGAGTATGACCAAGACCTCTGTGGACTGTACAATGTATATCTGTGGAGACAGCA
CAGAGTGTAGCAACCTGCTGCTCCAATATGGCTCCTTCTGTACCCAACTTAAgAGGGCTCTGACAGGC
ATTGCTGTGGAACAGGACAAGAACACCCAGGAGGTGTTTGCCCAGGTGAAGCAGATTTACAAGACACC
TCCAATCAAGtACTTTGGAGGCTTCAACTTCAGCCAGATTCTGCCTGACCCAAGCAAGCCAAGCAAGA
GGTCCTTCATTGAGGACCTGCTGTTCAACAAGGTGACCCTGGCTGATGCTGGCTTCATCAAGCAATAT
GGAGACTGTCTGGGAGACATTGCTGCCAGGGACCTGATTTGTGCCCAGAAGTTCAACGGACTGACAGT
GCTGCCTCCACTGCTGACAGATGAGATGATTGCCCAATACACCTCTGCCCTGCTGGCTGGCACCATCA
CCTCTGGCTGGACCTTTGGAGCAGGAGCAGCCCTCCAAATCCCATTTGCTATGCAGATGGCTTACAGG
TTCAATGGCATTGGAGTGACCCAGAATGTGCTCTATGAGAACCAGAAACTGATTGCCAACCAGTTCAA
CTCTGCCATTGGCAAGATTCAGGACTCCCTGTCCAGCACAGCCTCTGCCCTGGGCAAGCTCCAAGATG
TGGTGAACCACAATGCCCAGGCTCTGAACACCCTGGTGAAGCAACTTTCCAGCAAgTTTGGAGCCATC
TCCTCTGTGCTGAATGACATCCTGAGCAGACTGGACAAGGTGGAGGCTGAGGTCCAGATTGACAGACT
GATTACAGGCAGACTCCAATCCCTCCAAACCTATGTGACCCAACAACTTATCAGGGCTGCTGAGATTA
GGGCATCTGCCAACCTGGCTGCCACCAAGATGAGTGAGTGTGTGCTGGGACAAAGCAAGAGGGTGGAC
TTCTGTGGCAAGGGCTACCACCTGATGAGTTTTCCACAGTCTGCCCCTCATGGAGTGGTGTTCCTGCA
TGTGACCTATGTGCCTGCCCAGGAGAAGAACTTCACCACAGCCCCTGCCATCTGCCATGATGGCAAGG
CTCACTTTCCAAGGGAGGGAGTGTTTGTGAGCAATGGCACCCACTGGTTTGTGACCCAGAGGAACTTC
TATGAACCACAGATTATCACCACAGACAACACCTTTGTGTCTGGCAACTGTGATGTGGTGATTGGCAT
TGTGAACAACACAGTCTATGACCCACTCCAACCTGAACTGGACTCCTTCAAGGAGGAACTGGACAAAT
ACTTCAAGAACCACACCAGCCCTGATGTGGACCTGGGAGACATCTCTGGCATCAATGCCTCTGTGGTG
AACATCCAGAAGGAGATTGACAGACTGAATGAGGTGGCTAAGAACCTGAATGAGTCCCTGATTGACCT
CCAAGAACTGGGCAAATATGAACAATACATCAAGTGGCCATGGTACATCTGGCTGGGCTTCATTGCTG
GACTGATTGCCATTGTGATGGTGACCATAATGCTGTGTTGTATGACCTCCTGTTGTTCCTGTCTGAAA
GGCTGTTGTTCCTGTGGCTCCTGTTGTTGAggatccgagctcggtaccaagcttaagtttaaaccgct
gatcagcctcgactgtgccttctagttgccagccatctgttgtttgcccctcccccgtgccttccttg
accctggaaggtgccactcccactgtcctttcctaataaaatgaggaaattgcatcgcattgtctgag
taggtgtcattctattctggggggtggggtggggcaggacagcaagggggaggattgggaagacaata
gcaggcatgctggggatgcggtgggctctatggcttctgaggcggaaagaaccagctggggctctagg
gggtatccccacgcgccctgtagcggcgcattaagcgcggcgggtgtggtggttacgcgcagcgtgac
cgctacacttgccagcgccctagcgcccgctcctttcgctttcttcccttcctttctcgccacgttcg
ccggctttccccgtcaagctctaaatcgggggctccctttagggttccgatttagtgctttacggcac
ctcgaccccaaaaaacttgattagggtgatggttcacgtagtgggccatcgccctgatagacggtttt
tcgccctttgacgttggagtccacgttctttaatagtggactcttgttccaaactggaacaacactca
accctatctcggtctattcttttgatttataagggattttgccgatttcggcctattggttaaaaaat
gagctgatttaacaaaaatttaacgcgaattaattctgtggaatgtgtgtcagttagggtgtggaaag
tccccaggctccccagcaggcagaagtatgcaaagcatgcatctcaattagtcagcaaccaggtgtgg
aaagtccccaggctccccagcaggcagaagtatgcaaagcatgcatctcaattagtcagcaaccatag
tcccgcccctaactccgcccatcccgcccctaactccgcccagttccgcccattctccgccccatggc
tgactaattttttttatttatgcagaggccgaggccgcctctgcctctgagctattccagaagtagtg
aggaggcttttttggaggcctaggcttttgcaaaaagctcccgggagcttgtatatccattttcggat
ctgatcaagagacaggatgaggatcgtttcgcatgattgaacaagatggattgcacgcaggttctccg
gccgcttgggtggagaggctattcggctatgactgggcacaacagacaatcggctgctctgatgccgc
cgtgttccggctgtcagcgcaggggcgcccggttctttttgtcaagaccgacctgtccggtgccctga
atgaactgcaggacgaggcagcgcggctatcgtggctggccacgacgggcgttccttgcgcagctgtg
ctcgacgttgtcactgaagcgggaagggactggctgctattgggcgaagtgccggggcaggatctcct
gtcatctcaccttgctcctgccgagaaagtatccatcatggctgatgcaatgcggcggctgcatacgc
ttgatccggctacctgcccattcgaccaccaagcgaaacatcgcatcgagcgagcacgtactcggatg
gaagccggtcttgtcgatcaggatgatctggacgaagagcatcaggggctcgcgccagccgaactgtt
cgccaggctcaaggcgcgcatgcccgacggcgaggatctcgtcgtgacccatggcgatgcctgcttgc
cgaatatcatggtggaaaatggccgcttttctggattcatcgactgtggccggctgggtgtggcggac
cgctatcaggacatagcgttggctacccgtgatattgctgaagagcttggcggcgaatgggctgaccg
cttcctcgtgctttacggtatcgccgctcccgattcgcagcgcatcgccttctatcgccttcttgacg
agttcttctgagcgggactctggggttcgaaatgaccgaccaagcgacgcccaacctgccatcacgag
atttcgattccaccgccgccttctatgaaaggttgggcttcggaatcgttttccgggacgccggctgg
atgatcctccagcgcggggatctcatgctggagttcttcgcccaccccaacttgtttattgcagctta
taatggttacaaataaagcaatagcatcacaaatttcacaaataaagcatttttttcactgcattcta
gttgtggtttgtccaaactcatcaatgtatcttatcatgtctgtataccgtcgacctctagctagagc
ttggcgtaatcatggtcatagctgtttcctgtgtgaaattgttatccgctcacaattccacacaacat
acgagccggaagcataaagtgtaaagcctggggtgcctaatgagtgagctaactcacattaattgcgt
tgcgctcactgcccgctttccagtcgggaaacctgtcgtgccagctgcattaatgaatcggccaacgc
gcggggagaggcggtttgcgtattgggcgctcttccgcttcctcgctcactgactcgctgcgctcggt
cgttcggctgcggcgagcggtatcagctcactcaaaggcggtaatacggttatccacagaatcagggg
ataacgcaggaaagaacatgtgagcaaaaggccagcaaaaggccaggaaccgtaaaaaggccgcgttg
ctggcgtttttccataggctccgcccccctgacgagcatcacaaaaatcgacgctcaagtcagaggtg
gcgaaacccgacaggactataaagataccaggcgtttccccctggaagctccctcgtgcgctctcctg
ttccgaccctgccgcttaccggatacctgtccgcctttctcccttcgggaagcgtggcgctttctcat
agctcacgctgtaggtatctcagttcggtgtaggtcgttcgctccaagctgggctgtgtgcacgaacc
ccccgttcagcccgaccgctgcgccttatccggtaactatcgtcttgagtccaacccggtaagacacg
acttatcgccactggcagcagccactggtaacaggattagcagagcgaggtatgtaggcggtgctaca
gagttcttgaagtggtggcctaactacggctacactagaagaacagtatttggtatctgcgctctgct
gaagccagttaccttcggaaaaagagttggtagctcttgatccggcaaacaaaccaccgctggtagcg
gtggtttttttgtttgcaagcagcagattacgcgcagaaaaaaaggatctcaagaagatcctttgatc
ttttctacggggtctgacgctcagtggaacgaaaactcacgttaagggattttggtcatgagattatc
aaaaaggatcttcacctagatccttttaaattaaaaatgaagttttaaatcaatctaaagtatatatg
agtaaacttggtctgacagttaccaatgcttaatcagtgaggcacctatctcagcgatctgtctattt
cgttcatccatagttgcctgactccccgtcgtgtagataactacgatacgggagggcttaccatctgg
ccccagtgctgcaatgataccgcgagacccacgctcaccggctccagatttatcagcaataaaccagc
cagccggaagggccgagcgcagaagtggtcctgcaactttatccgcctccatccagtctattaattgt
tgccgggaagctagagtaagtagttcgccagttaatagtttgcgcaacgttgttgccattgctacagg
catcgtggtgtcacgctcgtcgtttggtatggcttcattcagctccggttcccaacgatcaaggcgag
ttacatgatcccccatgttgtgcaaaaaagcggttagctccttcggtcctccgatcgttgtcagaagt
aagttggccgcagtgttatcactcatggttatggcagcactgcataattctcttactgtcatgccatc
cgtaagatgcttttctgtgactggtgagtactcaaccaagtcattctgagaatagtgtatgcggcgac
cgagttgctcttgcccggcgtcaatacgggataataccgcgccacatagcagaactttaaaagtgctc
atcattggaaaacgttcttcggggcgaaaactctcaaggatcttaccgctgttgagatccagttcgat
gtaacccactcgtgcacccaactgatcttcagcatcttttactttcaccagcgtttctgggtgagcaa
aaacaggaaggcaaaatgccgcaaaaaagggaataagggcgacacggaaatgttgaatactcatactc
ttcctttttcaatattattgaagcatttatcagggttattgtctcatgagcggatacatatttgaatg
tatttagaaaaataaacaaataggggttccgcgcacatttccccgaaaagtgccacctgacgtc

### Results

As the immune protection conferred by first booster shot wanes over time and new Omicron subvariants emerge with stronger immune evasion, the need for variant-adapted coronavirus disease (COVID) vaccine booster is increasingly imminent. On Jun. 28, 2022 vaccine advisory committee of food and drug administration (FDA) voted in favor of updating COVID booster shot to add an Omicron component. However, the rapid displacement of dominant Omicron subvariants (from BA. 1 to BA.2, then BA.2.12.1 and now BA.4 and BA.5) poses a great challenge to update COVID vaccine targeting the fast-evolving variants while maintaining potency against circulating variants. Each former dominant Omicron strain, including BA.1, BA.2 and BA.2.12.1, drastically surges and subsides in a window of 3 months or even shorter. Omicron BA.4 and BA.5 subvariants emerge in April in Southern Africa and become dominant around the world since June this year. These Omicron sublineages quickly replace its predecessors in circumstances of existing herd immunity from vaccination or infection of past variants. Reinfection or breakthrough infection caused by new dominant variant is not uncommon due to its strong immune evasion, which complicates the redesign of new COVID boosters given the short time window of each Omicron wave and the lead time between design, validation, and deployment of new boosters.

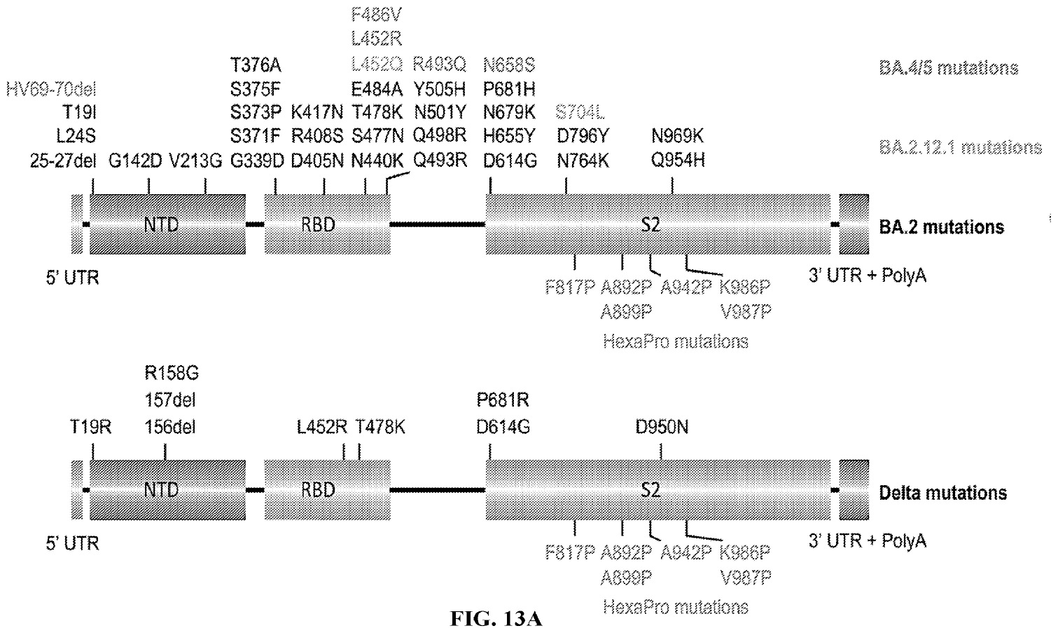

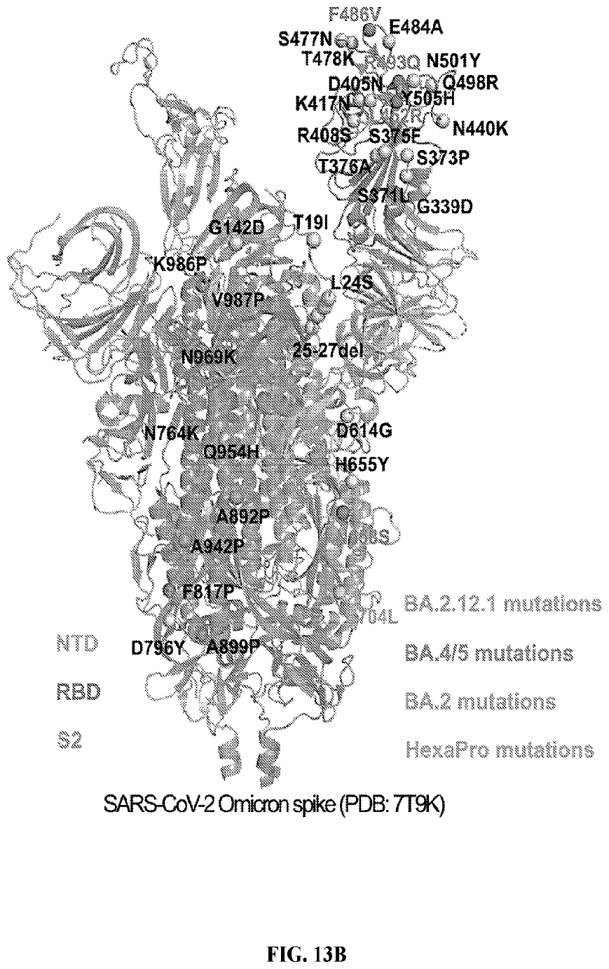

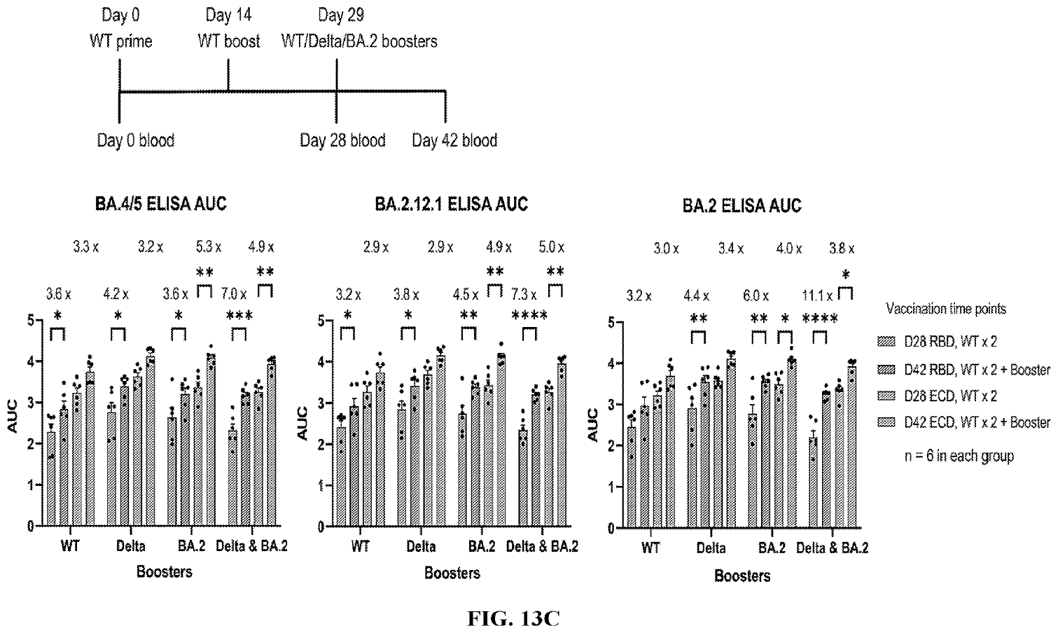

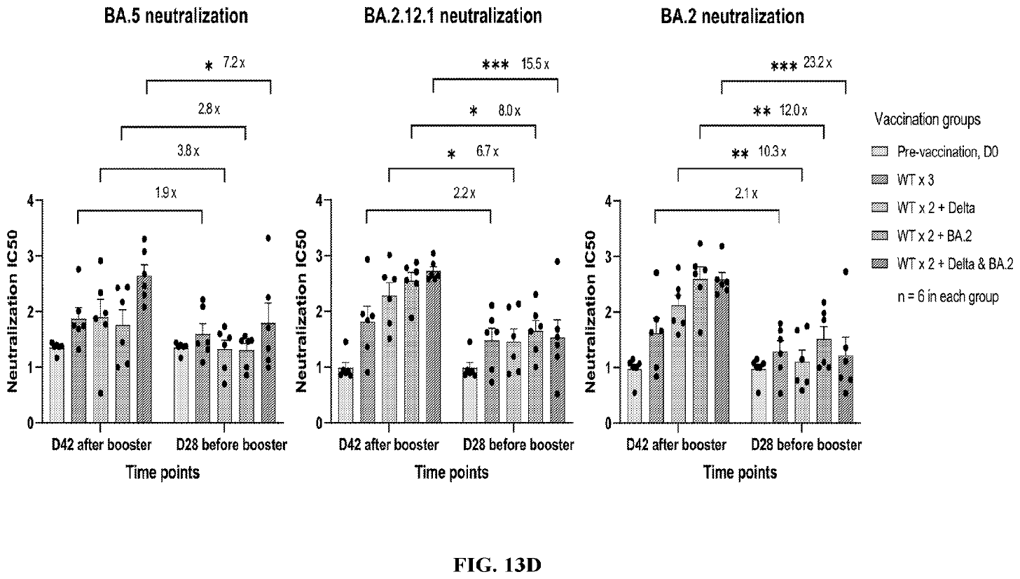

It is a crucial question to ask that which variant based antigen(s) to use in the next generation COVID boosters to elicit potent and broad response to past, present and emerging variants. At the time this study was initiated, the then-dominant subvariant BA.2 was gradually replaced by BA.2.12.1, BA.4 and BA.5. Compared to BA.2 spike, BA.2.12.1 contains two additional mutations (L452Q and S704L) while BA.4 and BA.5 spikes are identical and have 4 constant alterations (Del69-70, L452R, F486V, R493Q) plus one mutation (N658S) seen in earlier sequences (FIG. 13A-13B). The L452 substitutions in BA.2.12.1 and BA.4/5 are associated with neutralizing antibody escape and BA.4/5 combines the L452R mutation initially identified in Delta variant, highlighting one possible evolution trajectory of emerging variant by combining predecessors' beneficial mutations.

Bivalent vaccine candidates have gained recent tractions due to the concept of direct targeting of two variants, which may also induce broader immunity against other variants. Bivalent vaccine candidates have been under active clinical testing such as Modern's mRNA-1273.214, which is a equal mixture of two spike-encoding mRNAs with 25 μg targeting ancestral SARS-CoV-2 and 25 μg targeting the original Omicron Variant (B.1.1.529) (Moderna news releases Jun. 8, 2022 Jun. 22, 2022, and FDA committee meeting Jun. 28, 2022), demonstrating the importance and the clinically relevance of the concept of bivalent vaccination using two mRNAs. Considering this merge of variants' mutations (FIGS. 13A-13B), these studies sought to determine if mRNA vaccine candidates based on antigens of circulating variant (BA.2) and/or former dominant variant (Delta) can mediate broad antibody response to emerging variants such as BA.2.12.1, BA.4 or BA.5. It is worth to explore in this direction for a few reasons. The lead time of combining boosters adapted to dominant and former dominant variants will be shorter than predicting and developing boosters targeting new variants. In addition, because of the rapid displacement of circulating variants, the mismatch between the strain used for updated boosters and emerging strain may always exists. How to elicit broad response to emerging variants using existing variant antigens is an inevitable question to answer when redesigning updated COVID boosters.

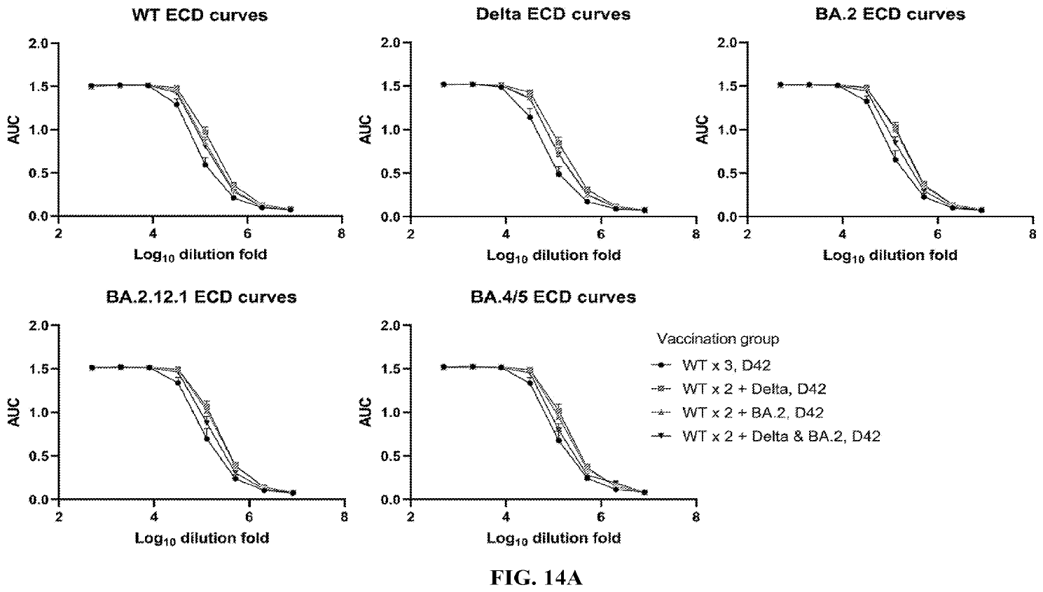

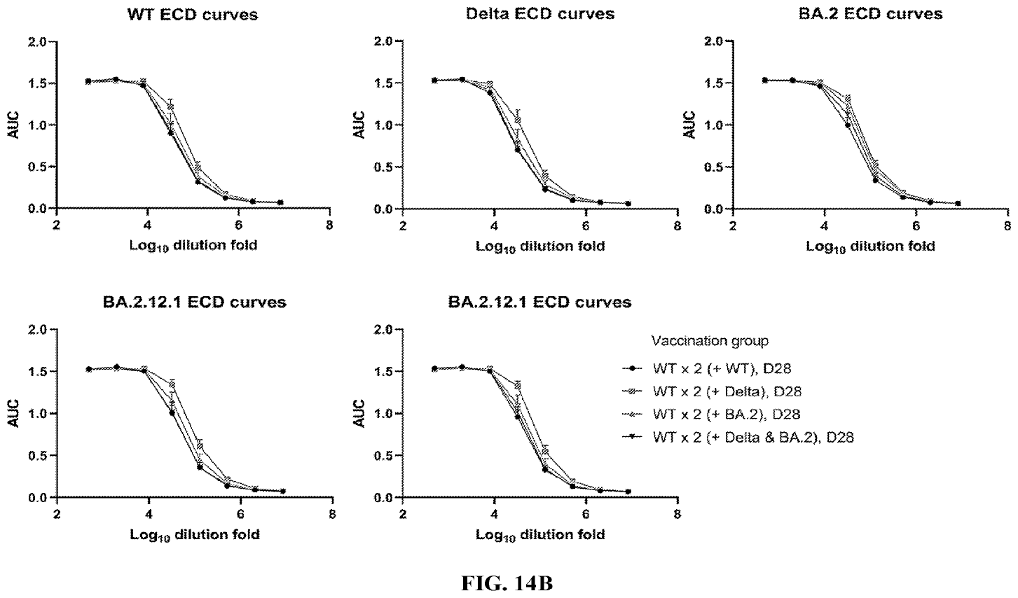

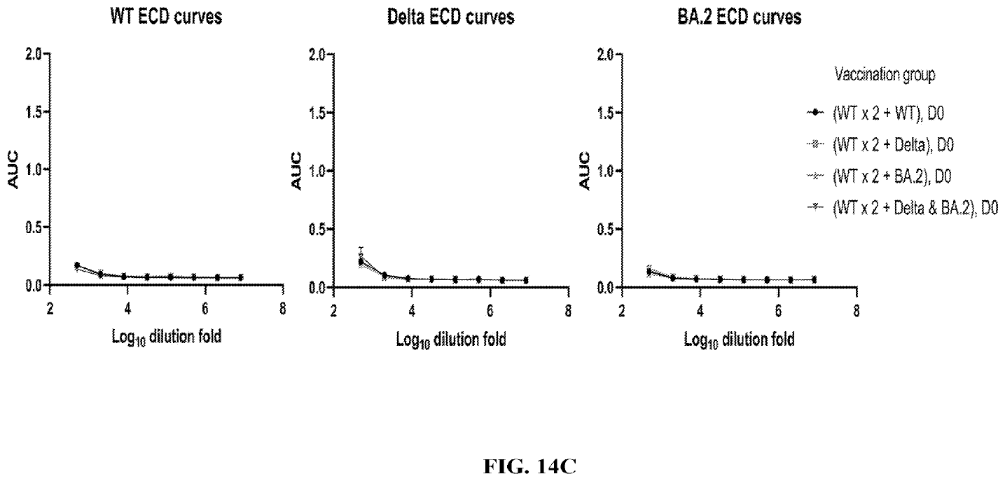

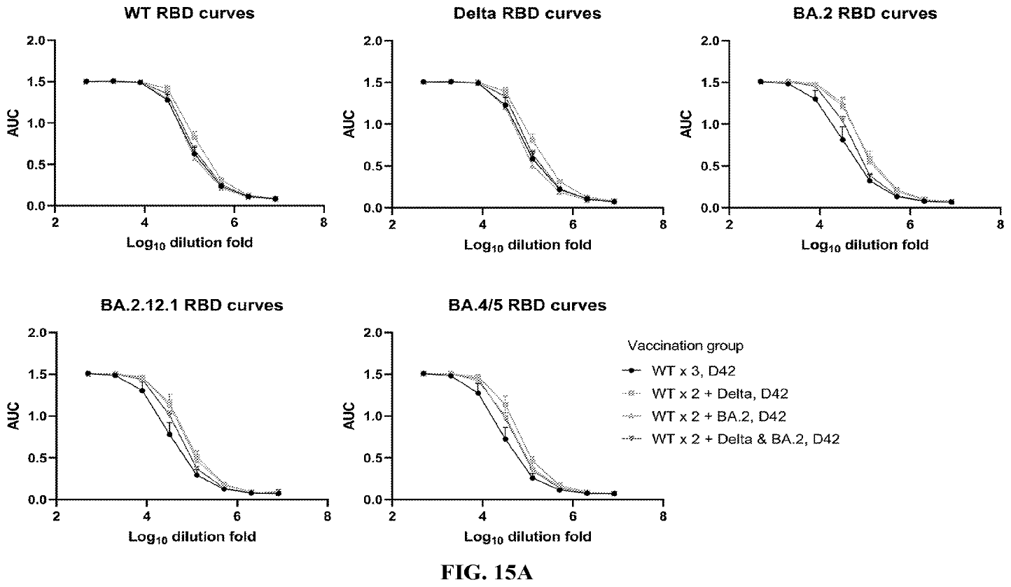

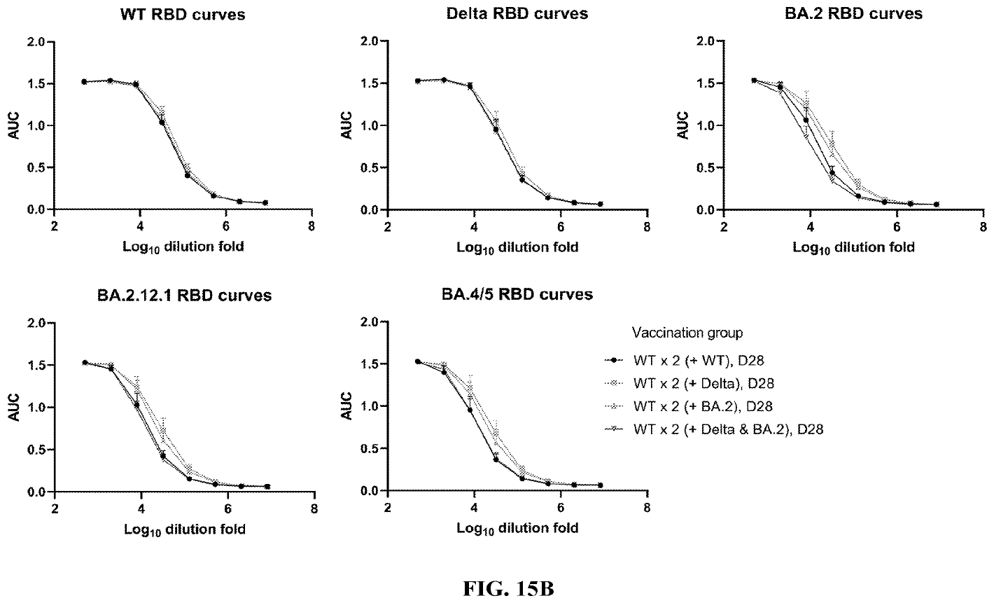

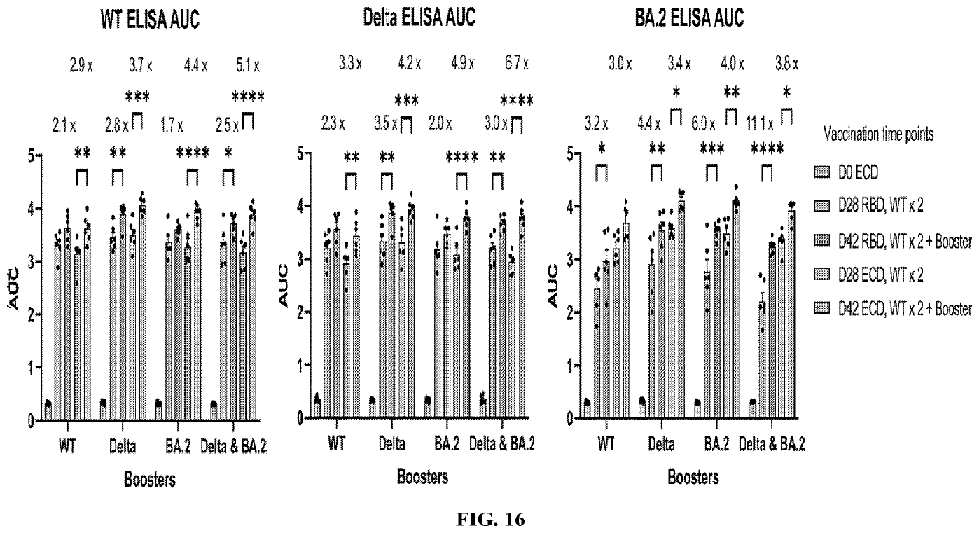

To answer this question, the antibody response elicited by ancestral (wild type, WT), Delta, BA.2 spike based monovalent or Delta & BA.2 bivalent mRNA boosters was compared to Omicron BA.2, BA.2.12.1 and BA.4/5 subvariants. In mice pre-immunized with two doses of WT lipid nanoparticle mRNA (LNP-mRNA), all three monovalent and one bivalent boosters elevated Omicron binding and neutralizing antibody titers to various degree in ELISA and pseudovirus neutralization assay (FIGS. 13C-13D and FIGS. 14-16), exemplifying the benefit of receiving WT or variant-adapted booster shots against circulating and emerging variants. Booster-associated titer ratios quantify booster's effect on antibody titers and were shown in each bar graph as post-booster titer on day 42 over pre-booster titer on day 28. Its dynamic range was greater in neutralization assay (ratio ranges from 3-23) than in ELISA (ratio ranges from 2-11).

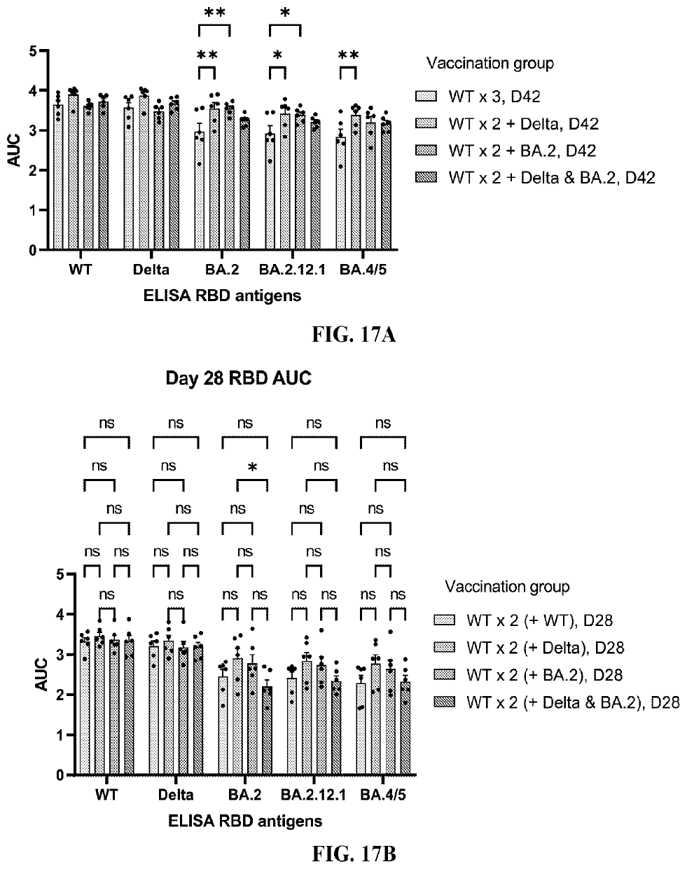

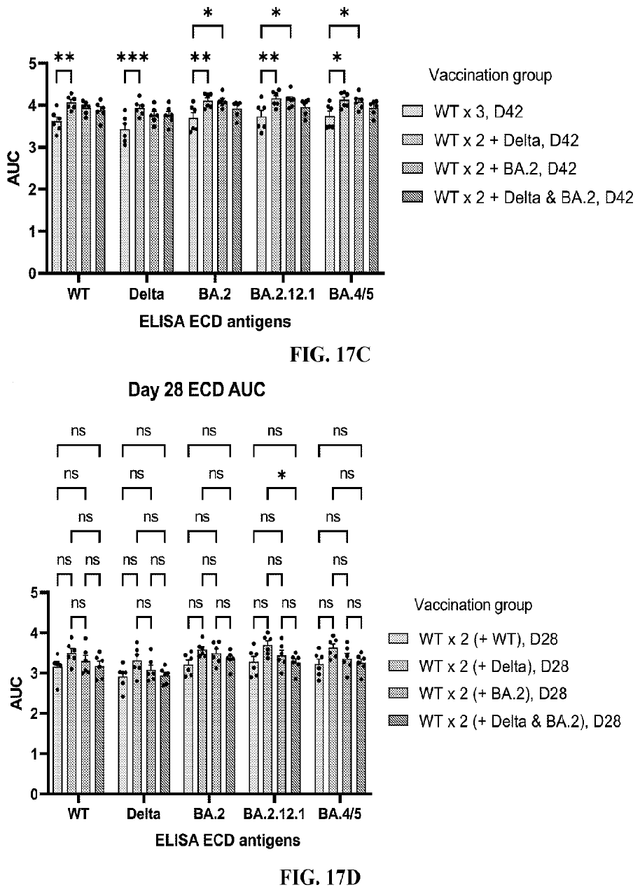

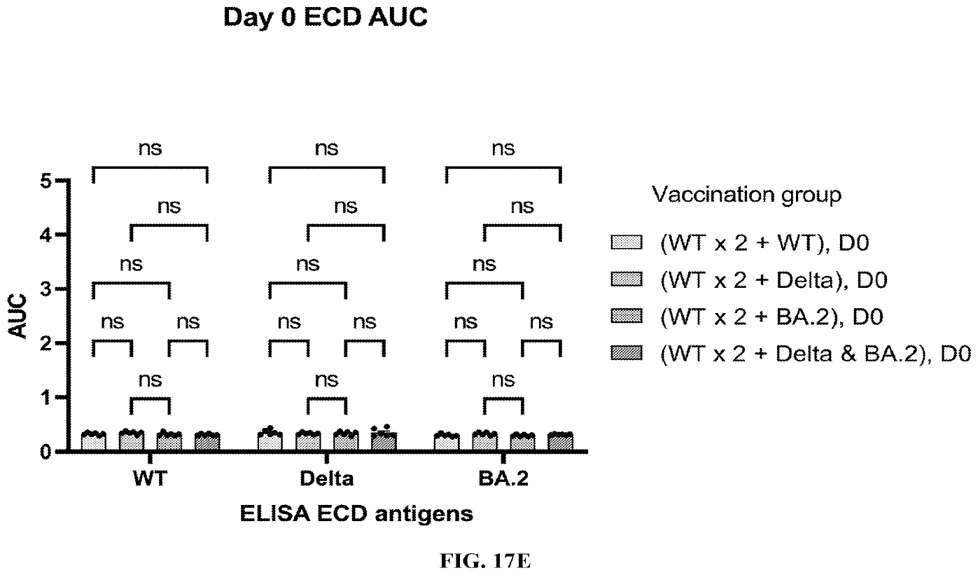

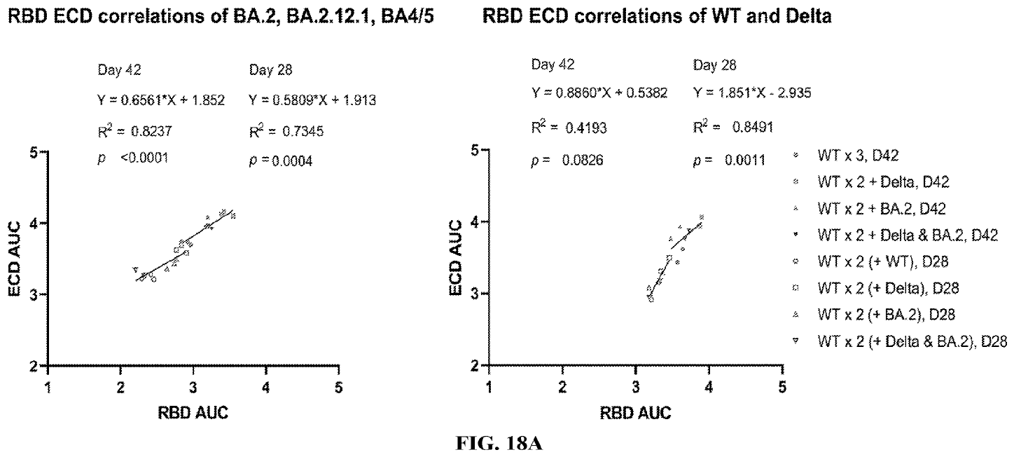

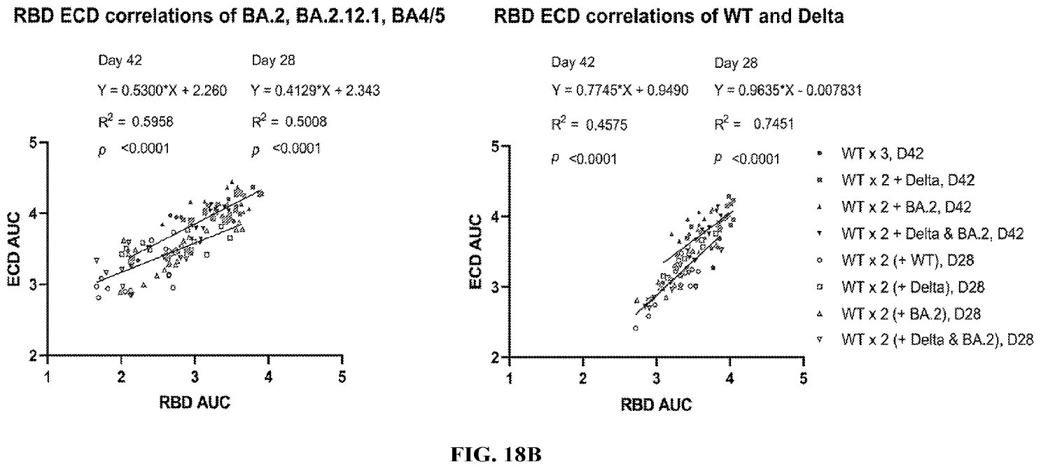

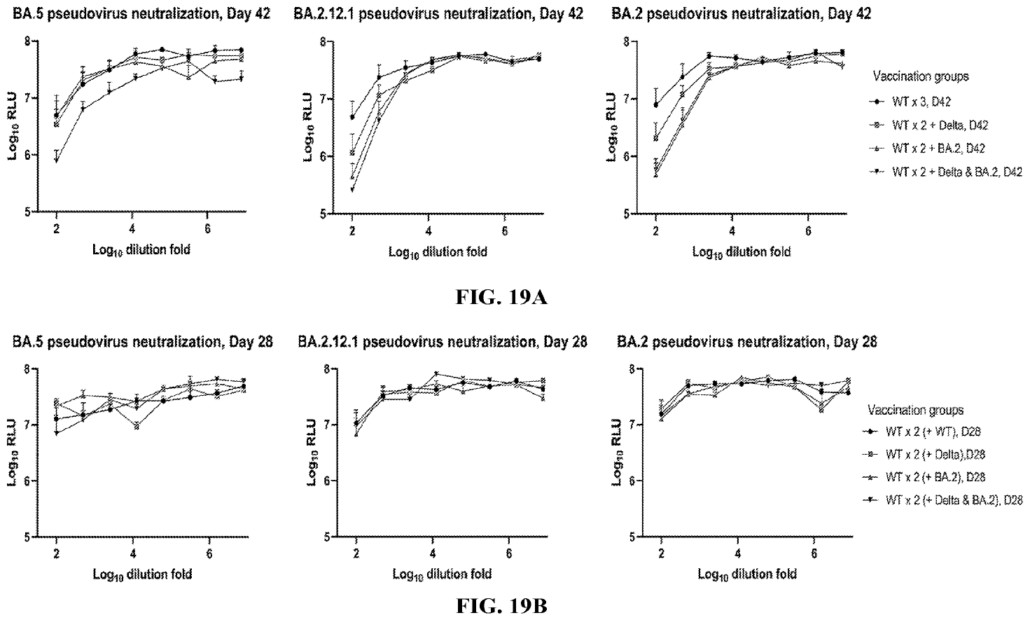

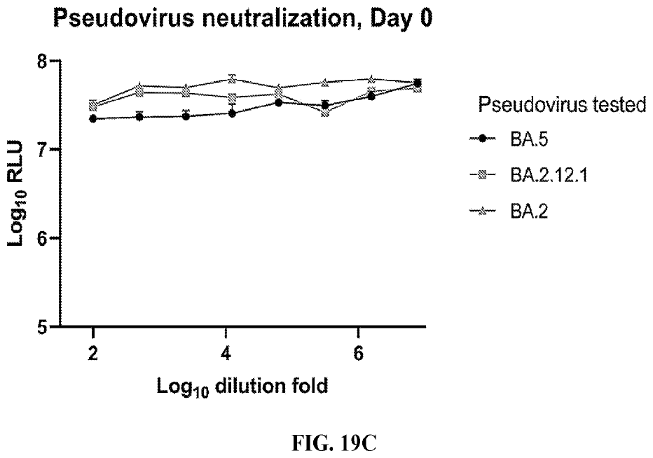

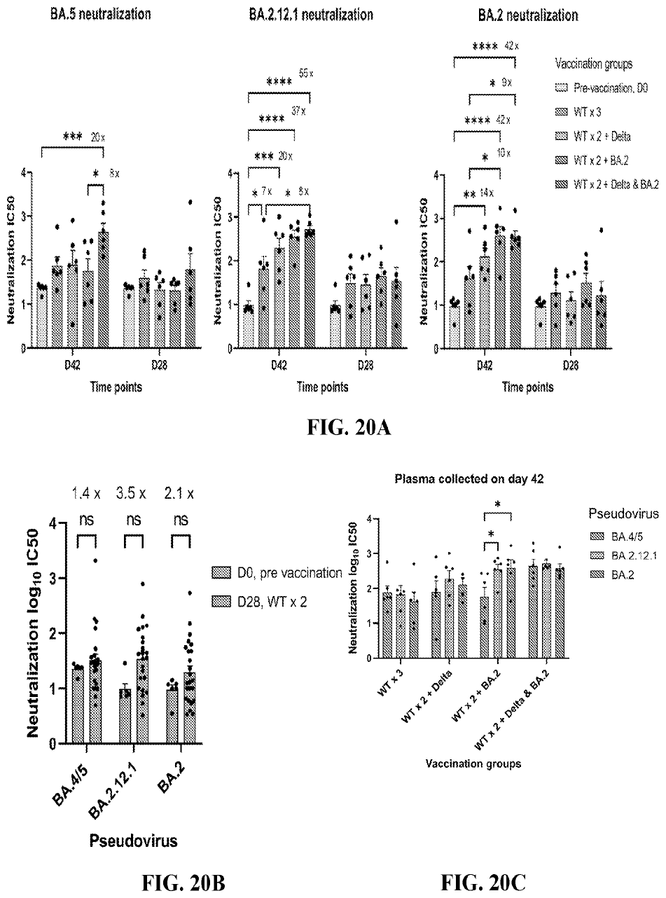

Before administered with different boosters, 24 mice in four groups received same treatment and showed little or no significant difference in antibody titers measured on day 0 and day 28 (FIGS. 17-19 and 20A). A moderate increase in Omicron neutralizing antibody titers was observed from immunization of two doses of WT LNP-mRNA (FIG. 20B). This titer increase by WT LNP-mRNA was lowest in neutralization assay of BA.4/5 (˜40% increase) as compared to BA.2.12.1 and BA.2. On day 42 two weeks post booster, the binding and neutralizing titers of WT booster group were frequently found lower than those of variant booster groups (FIGS. 17 and 20A), consistent with the fact that BA.4/5 have stronger evasion of existing antibody therapeutics or vaccine induced immunity. Interestingly, compared to the neutralizing titers of BA.2 and BA.2.12.1, BA.2 monovalent but not Delta & BA.2 bivalent booster suffered a significant loss of BA.4/5 neutralizing titer (FIG. 20C), indicative of broader activity of bivalent booster and strong neutralization escape of Omicron BA.4 or BA.5 even in the BA.2 mRNA vaccinated individuals. The RBD and ECD binding antibody titers were well correlated and showed distinct linear regression models between day 28 and day 42 as well as in WT, Delta (right panel in FIG. 18) and Omicron antigen datasets (left panel). The upper right shift of day 42 linear segment suggested a titer increase by boosters while the lower left shift in Omicron antigen dataset was associated with antibody evasion of Omicron antigens.

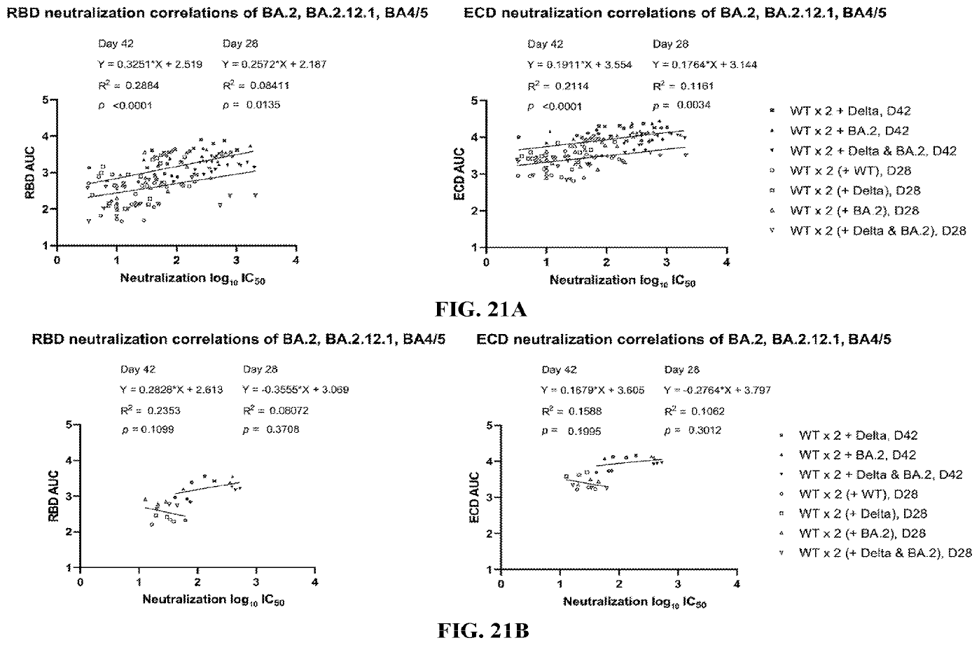

The boosting effect of Delta and BA.2 specific monovalent or bivalent LNP-mRNAs is universally higher than that of WT LNP-mRNA, which only modestly increased antibody titer (˜1 fold, fold change=ratio-1) in neutralization assays of Omicron BA.5, BA.2.12.1 and BA.2 (FIG. 13D). The Delta & BA.2 bivalent booster showed superior performance of enhancing binding and neutralizing titers than either monovalent counterparts, which is especially apparent in neutralization of Omicron BA.4 or BA.5. The bivalent booster associated titer ratios were 23-, 16- and 7-fold for neutralization of BA.2, BA.2.12.1 and BA.4/5, respectively while Delta/BA.2 monovalent booster ratios were 10/12, 7/8, 4/3 respectively. The linear regression models of neutralizing and binding titers showed a trend of correlation, but the goodness of fit was low due to deviations intrinsic in the two assays as well as heterogeneity stemmed from distinct boosters and Omicron subvariants tested (FIG. 21).

To sum up, and without wishing to be bound by theory, these data delivered a few clear messages regarding the potency of boosters against Omicron subvariants: 1) either WT or variant, monovalent or bivalent boosters can improve antibody response to Omicron BA.2, BA.2.12.1 and BA.4/5, demonstrating the benefit and necessity of receiving booster shots; 2) the variant boosters with closer antigenic distance to circulating variant perform universally better than WT booster; 3) compared to monovalent booster, bivalent booster combining two genetically distant variants, Delta & BA.2 showed broader and numerically stronger antibody response to Omicron BA.2, BA.2.12.1 and BA.4/5 subvariants. Taken together, this study presents a direct evaluation of Delta and BA.2 variant-adapted monovalent and bivalent mRNA boosters and compares their antibody response to Omicron subvariants with WT booster in the context of mouse model pre-immunized with two-dose WT LNP-mRNA vaccination. These data provide pre-clinical evidence and rationale for developing bivalent or multi-valent variant targeted COVID boosters.

### Example 7: Development of a Multiplexed LNP-mRNA Vaccination Against Pathogenic Coronavirus Species

### Materials and Methods

Animals. M. musculus (mice), 6-8 weeks old females of C57BL/6Ncr were purchased from Charles River. M. musculus (mice) used for immunogenicity study. Animals were housed in individually ventilated cages in a dedicated vivarium with clean food, water, and bedding. Animals are housed with a maximum of 5 mice per cage, at regular ambient room temperature (65-75° F., or 18-23° C.), 40-60% humidity, and a 14 h: 10 h light cycle. All experiments utilize randomized littermate controls.

Cell Lines. HEK293T (ThermoFisher), Huh-7 and 293T-hACE2 (Dr Bieniasz′ lab) cell lines were cultured in complete growth medium, Dulbecco's modified Eagle's medium (DMEM; ThermoFisher) supplemented with 10% Fetal bovine serum (FBS, Hyclone), 1% penicillin-streptomycin (Gibco) (D10 media for short). Cells were typically passaged every 1-2 days at a split ratio of 1:2 or 1:4 when the confluency reached about 80%.

Mouse immunization. 6-8 weeks old female C57BL/6Ncr (B6) mice were purchased from Charles River and used for vaccine immunogenicity study. Animals were housed in individually ventilated cages in a dedicated vivarium with clean food, water, and bedding. A maximum of 5 mice was allowed in each cage, at regular ambient room temperature (65-75° F., or 18-23° C.), 40-60% humidity, and a 14 h: 10 h day/night cycle. All experiments utilize randomized littermate controls. A standard two-dose schedule given 21 days apart was adopted (Polack et al., 2020), unless otherwise noted. Three sets of immunization experiments were performed: Triplex dosage testing, MERS Duplex testing and Schedule comparison testing.

For the Triplex dosage testing experiment, 1 μg Delta LNP-mRNA, 1 μg or 3 μg Triplex-CoV LNP-mRNA (equal mass mixture of Delta, MERS and SARS mRNA) were diluted to the same volume with 1×PBS and inoculated into mice intramuscularly during prime and boost.

For the MERS Duplex testing experiment, 3 μg MERS LNP-mRNA, 3 μg equal-mass mRNA mixture of MERS+SARS or MERS+Delta spikes at same concentration were inoculated into mice intramuscularly during prime and boost.

For the Schedule comparison testing experiment, 1 μg Delta, MERS and SARS LNP-mRNA were sequentially inoculated into mice during prime and boost. Control mice received 50 μl PBS at prime and boost at the same matched time points in all experiments.

Coronavirus spike sequence alignment. The spike sequence used to produce the LNP-mRNA vaccines were aligned using Clustal Omega (Goujon et al., 2010) and visualized in Jalview (Waterhouse et al., 2009).

Plasmid construction. The spike cDNA of SARS-CoV (Genbank accession AAP13567.1) and MERS-CoV (Genbank accession AFS88936.1) were purchased from Sino Biological (Cat #VG40150-G-N and VG40069-G-N, respectively). cDNA of SARS-CoV-2 B.1.617.2 (Delta variant) (Liu et al., 2021) were synthesized as gBlocks (IDT). The spike sequences were cloned by Gibson Assembly (NEB) into pcDNA3.1 plasmid for the mRNA transcription and pseudovirus assay. The plasmids for the pseudotyped virus assay including pHIVNLGagPol and pCCNanoLuc2AEGFP are gifts from Dr. Bieniasz′ lab (Schmidt et al., 2020). The C-terminal 19 (for SARS-CoV and SARS-CoV-2) or 16 (for MERS-CoV) amino acids were deleted in the spike sequence for the pseudovirus assay. To improve expression and retain prefusion conformation, six prolines (HexaPro variant, 6P) (Wrapp et al., 2020) were introduced to the SARS-CoV-2, SARS-CoV and MERS-CoV spike sequence at the homologous sites in the mRNA transcription plasmids. The furin site of SARS-CoV-2 spike (RRAR) were replaced with a GSAS short stretch to keep S1 and S2 subunits connected in the spike.

In vitro mRNA transcription and vaccine formulation. Codon-optimized mRNA encoding HexaPro spikes of SARS-CoV-2 WT, Delta, SARS-CoV and MERS-CoV were synthesized in vitro using an Hiscribe™ T7 ARCA mRNA Kit (with tailing) (NEB, Cat #E2060S), with 50% replacement of uridine by N1-methyl-pseudouridine. A linearized DNA template containing the spike open reading frame flanked by 5′ untranslated region (UTR), 3′ UTR and 3′-end polyA tail was used as for mRNA transcription. The linearization of DNA templates was achieved by digesting circular plasmids with BbsI restriction enzyme, followed by gel purification.

The mRNA was synthesized and purified by following the manufacturer's instructions and kept frozen at −80° C. until further use. In brief, the synthesized mRNA was purified by spin column-based method using Monarch RNA cleanup kit (NEB, Cat No. T2040L). The mRNA was encapsulated in lipid nanoparticles using the NanoAssemblr® Ignite™ machine (Precision Nanosystems). For the MixCoV vaccine, equal mass of SARS, MERS and Delta spike mRNA were mixed before encapsulated by lipid nanoparticles. All procedures are following the guidance of manufacturers. In brief, GenVoy ILM lipid mixture was mixed with transcribed mRNA in the low pH formulation buffer 1 on Ignite instrument at a molar ratio of 6:1 (LNP: mRNA), similar to previously described (Corbett et al., 2020; Hassett et al., 2019). The Gen Voy ILM contains 50% PNI ionizable lipids, 10% DSPC, 37.5% cholesterol and 2.5% PNI stabilizer. The LNP encapsulated mRNA was buffer exchanged to PBS using 30 kDa Amicon filter (MilliporeSigma™ UFC901024). Sucrose was added as a cryoprotectant. The particle size of mRNA-LNP was determined by DLS machine (DynaPro NanoStar, Wyatt, WDPN-06) and TEM described below. The encapsulation rate and mRNA concentration were measured by Quant-iT™ RiboGreen™ RNA Assay (ThermoFisher).

In vitro mRNA expression and receptor binding validation of translated spikes HEK293T cells were electroporated with mRNA encoding SARS, MERS or Delta spikes using Neon™ Transfection System 10 μL Kit following the standard protocol provided by manufacturer. After 12 h, the cells were collected and resuspended. To detect surface-protein expression, the cells were stained with ACE2-Fc chimera (Genscript, Z03484) or DPP4-Fc (Sino Biological, 10688-H01H) in MACS buffer (D-PBS with 2 mM EDTA and 0.5% BSA) for 30 min on ice. Thereafter, cells were washed twice and incubated with PE-anti-human FC antibody (Biolegend, 410708) in MACS buffer for 30 min on ice. Data acquisition was performed on BD FACSAria II Cell Sorter (BD). Analysis was performed using FlowJo software.

Negative-stain TEM. 5 μl of the sample was deposited on a glow-discharged formvar/carbon-coated copper grid (Electron Microscopy Sciences, catalog number FCF400-Cu-50), incubated for 1 min and blotted away. The grid was washed briefly with 2% (w/v) uranyl formate (Electron Microscopy Sciences, catalog number 22450) and stained for 1 min with the same uranyl formate buffer. Images were acquired using a JEOL JEM-1400 Plus microscope with an acceleration voltage of 80 kV and a bottom-mount 4k×3k charge-coupled device camera (Advanced Microscopy Technologies, AMT).

Sample collection, plasma and PBMCs isolation At the defined time points, usually two weeks post the last dose of boost unless otherwise noted (e.g. day 35, or day 119, as noted in the schematics), blood was retro-orbitally collected from mice. The PBMCs and plasma were isolated from blood via SepMate-15 (StemCell Technologies). 200 μl blood was immediately diluted with 800 μl PBS with 2% FBS. The diluted blood was then added to SepMate-15 tubes with 5 ml Lymphoprep (StemCell Technologies). 1200×g centrifugation for 20 minutes was applied to isolate RBCs, PBMCs and plasma. 200 μl diluted plasma was collected from the surface layer. Then the solution at the top layer containing PBMCs was poured to a new tube. PBMCs were washed once with PBS+2% FBS before being used in downstream analysis. The separated plasma was used in ELISA and neutralization assay. PBMCs were collected for single cell profiling using a 10× Genomics platform.

ELISA. The 384-well ELISA plates were coated with 3 μg/ml of antigens overnight at 4 degree. The antigen panel used in the ELISA assay includes SARS-CoV-2 spike S1+S2 ECD and RBD of 2019-nCoV WT (Sino Biological, ECD 40589-V08B1 and RBD 40592-V08B), Delta variant B.1.617.2 (SINO, ECD 40589-V08B16 and RBD 40592-V08H90), SARS-CoV (ECD Sino Biological 40634-V08B and RBD Fisher 50-196-4017) and MERS-CoV (ECD Sino Biological and RBD Fisher 50-201-9463). Plates were washed with PBS plus 0.5% Tween 20 (PBST) three times using the 50TS microplate washer (Fisher Scientific, NC0611021) and blocked with 0.5% BSA in PBST at room temperature for one hour. Plasma was serially diluted twofold or fourfold starting at a 1:500 dilution. Samples were added to the coated plates and incubate at room temperature for one hour, followed by washes with PBST five times. Anti-mouse secondary antibody (Fisher, Cat #A-10677) was diluted to 1:2500 in blocking buffer and incubated at room temperature for one hour. Plates were washed five times and developed with tetramethylbenzidine substrate (Biolegend, 421101). The reaction was stopped with 1 M phosphoric acid, and OD at 450 nm was determined by multimode microplate reader (PerkinElmer En Vision 2105). The binding response (OD450) were plotted against the dilution factor in log 10 scale to display the dilution-dependent response. The area under curve of the dilution-dependent response (Log 10 AUC) was calculated to evaluate the potency of the serum antibody binding to spike antigens.

Blocking ELISA. 0.6 μg/ml ECDs of Delta (Sino 40589-V08B16), MERS (40069-V08B) and SARS (Sino 40634-V08B) were coated to 384-well plate at 4 degree overnight. Low-density antigen was coated in blocking ELISA to ensure the blocking effect can be observed. The coated plate was then washed with PBST (0.5% Tween-20) three times and blocked with 2% BSA in PBST for 1 hour at room temperature. Equal volume of blocking agents at 5 μg/ml was mixed with serially diluted plasma and incubated at room temperature for 30 min before added to the plate. The blocking agents include PBS as negative control, Delta ECD, SARS ECD or MERS ECD. The conditions used in blocking ELISA was based on the optimized competition ELISA conditions in our previous study (Fang et al., 2022). After 1 hour incubation with the plasma and blocking agents, the plate was washed with PBST 5 times and incubated with anti-mouse secondary antibody (Fisher, Cat #A-10677) for 1 hour. Then the plate was washed five times with PBST, developed with tetramethylbenzidine substrate and fixed with 1M phosphoric acid. The OD450 was quantified by multimode microplate reader (PerkinElmer En Vision 2105). The normalized blocking effect was calculated by normalizing the AUC reduction by blocking reagents with AUC difference between plasma samples of PBS and vaccination groups.

Pseudovirus neutralization assay. HIV-1 based SARS-CoV-2 WT, B.1.617.2 (delta) variant, SARS and MERS pseudotyped virions were generated using corresponding spike sequences, and applied in neutralization assays. The pseudotyped virus was packaged using a coronavirus spike plasmid, a reporter vector and a HIV-1 structural protein expression plasmid. The reporter vector, pCCNanoLuc2AEGFP, and HIV-1 structural/regulatory proteins (pHIVNLGagPol) expression plasmid were from Bieniasz lab. The spike plasmid for SARS-CoV-2 WT pseudovirus truncated 19 C-terminal amino acids of S protein (SARS-CoV-2-Δ19) and was from Bieniasz lab. Spike plasmids expressing C-terminally truncated SARS-CoV-2 B.1.617.2 variant S protein (Delta variant-Δ19), SARS-CoV S protein (SARS-CoV-Δ19) and MERS S protein (MERS-CoV-Δ16) were generated based on the pSARS-CoV-2-Δ19. Briefly, 293T cells were seeded in 150 mm plates, and transfected with 21 μg pHIVNLGagPol, 21 μg pCCNanoLuc2AEGFP, and 7.5 μg of corresponding spike plasmids, in the presence of 198 μl PEI. At 48 h after transfection, the 20-ml supernatant was harvested and filtered through a 0.45-μm filter, and concentrated before aliquoted and frozen in −80° C.

The SARS-CoV and SARS-CoV-2 pseudovirus neutralization assays were performed on 293T-hACE2 cell, while the MERS-CoV neutralization assay was performed on Huh-7 cells. One day before infection, 293T-hACE2 cells were plated in a 96 well plate with 0.01×106 cells per well. In the next day, plasma collected from PBS or LNP-mRNA immunized mice were 5-fold serially diluted with complete growth medium starting from 1:100. 55 μL aliquots of diluted plasma were mixed with the same volume of SARS-CoV-2 WT, Delta variant, SARS or MERS pseudovirus. The mixture was incubated for 1 hr in the 37° C. incubator, supplied with 5% CO2. Then 100 μL of mixtures were added into 96-well plates with 293T-hACE2 or Huh-7 cells. Plates were incubated at 37° C. for 48 hr. Then host cells were collected and the percent of GFP-positive cells were analyzed with Attune N×T Acoustic Focusing Cytometer (ThermoFisher). The 50% inhibitory concentration (IC50) was calculated with a four-parameter logistic regression using GraphPad Prism (GraphPad Software Inc.). If the curve of individual mouse fails to produce positive fit (i.e. negative titer), suggestive of no neutralization activity, the value was converted to zero.

Authentic virus neutralization assay. Mouse plasma samples were serially diluted, then incubated with SARS-CoV-2 isolate USA-WA1/2020 for 1 h at 37° C. Vero-E6 overexpressing ACE2/TMPRSS2 was added to the plasma/virus mixture such that the final MOI was 1. Cell viability was measured at 72 hpi using CellTiter Glo.

Correlation analysis. Correlation analysis of ELISA, pseudovirus neutralization and authentic virus neutralization data were performed using the respective data collected. Linear regression model was used to evaluate the correlations between ELISA RBD and ECD AUCs, pseudovirus neutralization and authentic virus neutralization log 10 IC50. Model fitting and statistical analysis were performed in Graphpad Prism9.1.2. Correlations of data points from either individual mouse, or group average of different vaccination groups, were analyzed separately. The vaccination-group ELISA AUC or neutralization log 10 IC50 were calculated from the average of individual value in each group. Due to assay-dependent PBS background level, only non-PBS data points were included in the correlation analysis.

Single cell RNA-seq. PBMCs were collected from mRNA-LNP vaccinated and control mice were collected as described above for mouse immunization and sample collection, and normalized to 1000 cells/μL. Standard volumes of cell suspension were loaded to achieve targeted cell recovery to 10000 cells. The samples were subjected to 14 cycles of cDNA amplification. Following this, gene expression (GEX) libraries were prepared according to the manufacturer's protocol (10× Genomics). All libraries were sequenced using a NovaSeq 6000 (Illumina) with 2*150 read length.

Single cell data analysis for immune repertoire profiling and transcriptomic signatures. Both standard established pipelines and custom scripts were used for processing and analyzing single cell GEX data. Illumina sequencing data was processed using the Cellranger v6.0.1 (10× Genomics) pipeline, aligning reads to the mm10 reference transcriptome and aggregating all samples. Cellranger outputs were then preprocessed using a modified Seurat v4.0.5 workflow with the R statistical programming language (Satija et al., 2015). Briefly, individual sample data sets were filtered for quality cells (200-2000 RNA features and <5% mitochondrial RNA), log-normalized, scaled, and quality features were selected to calculate low-dimensional “anchors” (reciprocal-PCR dimensional reduction, k=20, anchors=2000), which were used to integrate the different sample data sets (Stuart et al., 2019). Integrated single-cell data were scaled, centered, clustered by shared nearest neighbors graph (k=20, first 12 PCA dimensions, chosen by the elbow plot method) with modularity optimization (Louvain algorithm with multilevel refinement, empirically chosen resolution=0.31). Clustered cells were visualized in low-dimensional space by uniform manifold approximation and projection (UMAP; first 12 PCA dimensions) (McInnes et al., 2018), and clusters were labeled as immune cell types via canonical marker expression, based on scaled-mean expression and expression detection rate for the cluster. Immune cell subtypes were identified for B cells, plasma cells, activated CD4 T cells, and mononuclear myeloid cells by sub-setting the cells of each group, rescaling with mt-RNA % as a covariate, centering, UMAP dimensional reduction as before (first 14, 11, 16, and 10 PCA dimensions for B cells, plasma cells, activated CD4 T cells, and myeloid cells, respectively), and clustering was performed as previously described (empirically chosen modularity resolution=0.20, 0.10, 0.25, and 0.10 for B cells, plasma cells, activated CD4 T cells, and myeloid cells, respectively), but canonical marker genes were used as features. To show that the cell type populations displayed distinct transcriptional profiles, markers were identified for each cluster vs all other cells using Wilcoxon rank sum testing of scaled data (SeuratWrappers::RunPrestoAll R function), while down-sampling to 5000 cells per cluster. The top 10 mean log fold change genes were selected from each cell type to visualize by heatmap with hierarchical clustering.

Differential expression was performed using the edgeR analysis pipeline and quasi-likelihood (QL) F tests. Specifically, raw single-cell expression data was filtered to include genes with >5% detection rate across all cells, genes were TMM-normalized, fitted to a QL negative binomial generalized linear model using trended dispersion estimates with cell detection rate and treatment as covariates, and empirical Bayes QL F tests were performed with treatment as the coefficient equal to zero under the null hypothesis.

Pathway enrichment analyses were performed for differentially expressed genes (DEG; absolute log 2 (x+1) expression fold-change >0.5, FDR-adjusted p value (q)<0.01) using the gost function of the gProfiler2 R package with biological process gene ontologies (GO) for Mus musculus, an adjusted p value-ordered gene list, and known genes as the domain for the statistics. In addition, the analysis p values were adjusted for multiple testing using the gProfiler gSCS method. Results were filtered to include GO terms <=600 genes in size that intersected >2 DEG, an absolute activation score (mean log 2 (x+1) expression fold change of GO term DEGs)>0.5, and an adjusted p<0.01. Network analyses were performed by (1) creating network graphs with filtered pathway results as nodes and GO term similarity coefficients as edges (coefficients=50% jaccard+50% overlap scores; edge similarity threshold=0.375), (2) finding graph clusters via the Leiden algorithm using the modularity method with similarity coefficients as weights (resolution=0.5, iterations=1000), and (3) labeling clusters by their most significant GO term (meta-pathway). Meta-pathway genes were visualized by heatmap, using log-normalized, scaled expression for GO term genes that were differentially expressed in vaccination groups compared to the PBS control. Custom R scripts were used for generating various plots.

Before the COVID-19 pandemic, no effective vaccine had been approved to prevent spread of coronaviruses. Previous SARS and MERS vaccine devolvement, although at earlier stages, together with global efforts, led to rapid development of multiple COVID-19 vaccines against SARS-CoV-2. The most prominent and efficacious vaccine belong to the lipid nanoparticle (LNP) mRNA vaccine category, with the first two emergency use approval issued to Moderna and Pfizer-BioNTech mRNA vaccines. Although successful vaccines against SARS-CoV-2 have been developed to control COVID-19, no effective vaccines exist that can counter multiple pathogenic coronavirus species including SARS-CoV and MERS-CoV. Thus, it is important to develop multi-species coronavirus vaccines, not only to help fight the ongoing pandemic, but also to prevent reemergence of these previously existed dangerous pathogens, as well as to gain insights to prepare for future zoonotic pathogenic coronavirus outbreaks.

The success of LNP-mRNA vaccine against COVID-19 led to the natural hypothesis of multiplexed vaccination against multiple coronavirus species. In the studies presented herein, species-specific LNP-mRNA vaccine candidates were generated and tested either alone or in combination in vivo. LNP-mRNAs were generated specifically encoding the HexaPro engineered full-length spikes of SARS-CoV-2 Delta variant, SARS-CoV and MERS-CoV, and systematically studied their immune responses in animal models.

Standard statistics. Standard statistical methods were applied to non-high-throughput experimental data. The statistical methods are described in figure legends and/or supplementary Excel tables. The statistical significance was labeled as follows: n.s., not significant; * p<0.05; ** p<0.01; *** p<0.001; **** p<0.0001. Prism (GraphPad Software) and RStudio were used for these analyses. Additional information can be found in the supplemental excel tables.

PVP24 pcDNA3.1 Spike del19
(SEQ ID NO: 38)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccATGTTTGTGTTCCTGGTGCTGCTGCCACTGGTGTCCAGCCAGTGTGTG
AACCTGACCACCAGGACCCAACTTCCTCCTGCCTACACCAACTCCTTCACCAGGGGAGTCTACTACCC
TGACAAGGTGTTCAGGTCCTCTGTGCTGCACAGCACCCAGGACCTGTTCCTGCCATTCTTCAGCAATG
TGACCTGGTTCCATGCCATCCATGTGTCTGGCACCAATGGCACCAAGAGGTTTGACAACCCTGTGCTG
CCATTCAATGATGGAGTCTACTTTGCCAGCACAGAGAAGAGCAACATCATCAGGGGCTGGATTTTTGG
CACCACCCTGGACAGCAAGACCCAGTCCCTGCTGATTGTGAACAATGCCACCAATGTGGTGATTAAGG
TGTGTGAGTTCCAGTTCTGTAATGACCCATTCCTGGGAGTCTACTACCACAAGAACAACAAGTCCTGG
ATGGAGTCTGAGTTCAGGGTCTACTCCTCTGCCAACAACTGTACCTTTGAATATGTGAGCCAACCATT
CCTGATGGACTTGGAGGGCAAGCAGGGCAACTTCAAGAACCTGAGGGAGTTTGTGTTCAAGAACATTG
ATGGCTACTTCAAGATTTACAGCAAACACACACCAATCAACCTGGTGAGGGACCTGCCACAGGGCTTC
TCTGCCTTGGAACCACTGGTGGACCTGCCAATTGGCATCAACATCACCAGGTTCCAGACCCTGCTGGC
TCTGCACAGGTCCTACCTGACACCTGGAGACTCCTCCTCTGGCTGGACAGCAGGAGCAGCAGCCTACT
ATGTGGGCTACCTCCAACCAAGGACCTTCCTGCTGAAATACAATGAGAATGGCACCATCACAGATGCT
GTGGACTGTGCCCTGGACCCACTGTCTGAGACCAAGTGTACCCTGAAATCCTTCACAGTGGAGAAGGG
CATCTACCAGACCAGCAACTTCAGGGTCCAACCAACAGAGAGCATTGTGAGGTTTCCAAACATCACCA
ACCTGTGTCCATTTGGAGAGGTGTTCAATGCCACCAGGTTTGCCTCTGTCTATGCCTGGAACAGGAAG
AGGATTAGCAACTGTGTGGCTGACTACTCTGTGCTCTACAACTCTGCCTCCTTCAGCACCTTCAAGTG
TTATGGAGTGAGCCCAACCAAACTGAATGACCTGTGTTTCACCAATGTCTATGCTGACTCCTTTGTGA
TTAGGGGAGATGAGGTGAGACAGATTGCCCCTGGACAAACAGGCAAGATTGCTGACTACAACTACAAA
CTGCCTGATGACTTCACAGGCTGTGTGATTGCCTGGAACAGCAACAACCTGGACAGCAAGGTGGGAGG
CAACTACAACTACCTCTACAGACTGTTCAGGAAGAGCAACCTGAAACCATTTGAGAGGGACATCAGCA
CAGAGATTTACCAGGCTGGCAGCACACCATGTAATGGAGTGGAGGGCTTCAACTGTTACTTTCCACTC
CAATCCTATGGCTTCCAACCAACCAATGGAGTGGGCTACCAACCATACAGGGTGGTGGTGCTGTCCTT
TGAACTGCTCCATGCCCCTGCCACAGTGTGTGGACCAAAGAAGAGCACCAACCTGGTGAAGAACAAGT
GTGTGAACTTCAACTTCAATGGACTGACAGGCACAGGAGTGCTGACAGAGAGCAACAAGAAGTTCCTG
CCATTCCAACAGTTTGGCAGGGACATTGCTGACACCACAGATGCTGTGAGGGACCCACAGACCTTGGA
GATTCTGGACATCACACCATGTTCCTTTGGAGGAGTGTCTGTGATTACACCTGGCACCAACACCAGCA
ACCAGGTGGCTGTGCTCTACCAGGATGTGAACTGTACTGAGGTGCCTGTGGCTATCCATGCTGACCAA
CTTACACCAACCTGGAGGGTCTACAGCACAGGCAGCAATGTGTTCCAGACCAGGGCTGGCTGTCTGAT
TGGAGCAGAGCATGTGAACAACTCCTATGAGTGTGACATCCCAATTGGAGCAGGCATCTGTGCCTCCT
ACCAGACCCAGACCAACAGCCCAAGGAGGGCAAGGTCTGTGGCAAGCCAGAGCATCATTGCCTACACA
ATGAGTCTGGGAGCAGAGAACTCTGTGGCTTACAGCAACAACAGCATTGCCATCCCAACCAACTTCAC
CATCTCTGTGACCACAGAGATTCTGCCTGTGAGTATGACCAAGACCTCTGTGGACTGTACAATGTATA
TCTGTGGAGACAGCACAGAGTGTAGCAACCTGCTGCTCCAATATGGCTCCTTCTGTACCCAACTTAAC
AGGGCTCTGACAGGCATTGCTGTGGAACAGGACAAGAACACCCAGGAGGTGTTTGCCCAGGTGAAGCA
GATTTACAAGACACCTCCAATCAAGGACTTTGGAGGCTTCAACTTCAGCCAGATTCTGCCTGACCCAA
GCAAGCCAAGCAAGAGGTCCTTCATTGAGGACCTGCTGTTCAACAAGGTGACCCTGGCTGATGCTGGC
TTCATCAAGCAATATGGAGACTGTCTGGGAGACATTGCTGCCAGGGACCTGATTTGTGCCCAGAAGTT
CAATGGACTGACAGTGCTGCCTCCACTGCTGACAGATGAGATGATTGCCCAATACACCTCTGCCCTGC
TGGCTGGCACCATCACCTCTGGCTGGACCTTTGGAGCAGGAGCAGCCCTCCAAATCCCATTTGCTATG
CAGATGGCTTACAGGTTCAATGGCATTGGAGTGACCCAGAATGTGCTCTATGAGAACCAGAAACTGAT
TGCCAACCAGTTCAACTCTGCCATTGGCAAGATTCAGGACTCCCTGTCCAGCACAGCCTCTGCCCTGG
GCAAACTCCAAGATGTGGTGAACCAGAATGCCCAGGCTCTGAACACCCTGGTGAAGCAACTTTCCAGC
AACTTTGGAGCCATCTCCTCTGTGCTGAATGACATCCTGAGCAGACTGGACAAGGTGGAGGCTGAGGT
CCAGATTGACAGACTGATTACAGGCAGACTCCAATCCCTCCAAACCTATGTGACCCAACAACTTATCA
GGGCTGCTGAGATTAGGGCATCTGCCAACCTGGCTGCCACCAAGATGAGTGAGTGTGTGCTGGGACAA
AGCAAGAGGGTGGACTTCTGTGGCAAGGGCTACCACCTGATGAGTTTTCCACAGTCTGCCCCTCATGG
AGTGGTGTTCCTGCATGTGACCTATGTGCCTGCCCAGGAGAAGAACTTCACCACAGCCCCTGCCATCT
GCCATGATGGCAAGGCTCACTTTCCAAGGGAGGGAGTGTTTGTGAGCAATGGCACCCACTGGTTTGTG
ACCCAGAGGAACTTCTATGAACCACAGATTATCACCACAGACAACACCTTTGTGTCTGGCAACTGTGA
TGTGGTGATTGGCATTGTGAACAACACAGTCTATGACCCACTCCAACCTGAACTGGACTCCTTCAAGG
AGGAACTGGACAAATACTTCAAGAACCACACCAGCCCTGATGTGGACCTGGGAGACATCTCTGGCATC
AATGCCTCTGTGGTGAACATCCAGAAGGAGATTGACAGACTGAATGAGGTGGCTAAGAACCTGAATGA
GTCCCTGATTGACCTCCAAGAACTGGGCAAATATGAACAATACATCAAGTGGCCATGGTACATCTGGC
TGGGCTTCATTGCTGGACTGATTGCCATTGTGATGGTGACCATAATGCTGTGTTGTATGACCTCCTGT
TGTTCCTGTCTGAAAGGCTGTTGTTCCTGTGGCTCCTGTTGTTGAggatccgagctcggtaccaagct
taagtttaaaccgctgatcagcctcgactgtgccttctagttgccagccatctgttgtttgcccctcc
cccgtgccttccttgaccctggaaggtgccactcccactgtcctttcctaataaaatgaggaaattgc
atcgcattgtctgagtaggtgtcattctattctggggggtggggggggcaggacagcaagggggagg
attgggaagacaatagcaggcatgctggggatgcggtgggctctatggcttctgaggcggaaagaacc
agctggggctctagggggtatccccacgcgccctgtagcggcgcattaagcgcggcgggtgtggtggt
tacgcgcagcgtgaccgctacacttgccagcgccctagcgcccgctcctttcgctttcttcccttcct
ttctcgccacgttcgccggctttccccgtcaagctctaaatcgggggctccctttagggttccgattt
agtgctttacggcacctcgaccccaaaaaacttgattagggtgatggttcacgtagtgggccatcgcc
ctgatagacggtttttcgccctttgacgttggagtccacgttctttaatagtggactcttgttccaaa
ctggaacaacactcaaccctatctcggtctattcttttgatttataagggattttgccgatttcggcc
tattggttaaaaaatgagctgatttaacaaaaatttaacgcgaattaattctgtggaatgtgtgtcag
ttagggtgtggaaagtccccaggctccccagcaggcagaagtatgcaaagcatgcatctcaattagtc
agcaaccaggtgtggaaagtccccaggctccccagcaggcagaagtatgcaaagcatgcatctcaatt
agtcagcaaccatagtcccgcccctaactccgcccatcccgcccctaactccgcccagttccgcccat
tctccgccccatggctgactaattttttttatttatgcagaggccgaggccgcctctgcctctgagct
attccagaagtagtgaggaggcttttttggaggcctaggcttttgcaaaaagctcccgggagcttgta
tatccattttcggatctgatcaagagacaggatgaggatcgtttcgcatgattgaacaagatggattg
cacgcaggttctccggccgcttgggtggagaggctattcggctatgactgggcacaacagacaatcgg
ctgctctgatgccgccgtgttccggctgtcagcgcaggggcgcccggttctttttgtcaagaccgacc
tgtccggtgccctgaatgaactgcaggacgaggcagcgcggctatcgtggctggccacgacgggcgtt
ccttgcgcagctgtgctcgacgttgtcactgaagcgggaagggactggctgctattgggcgaagtgcc
ggggcaggatctcctgtcatctcaccttgctcctgccgagaaagtatccatcatggctgatgcaatgc
ggcggctgcatacgcttgatccggctacctgcccattcgaccaccaagcgaaacatcgcatcgagcga
gcacgtactcggatggaagccggtcttgtcgatcaggatgatctggacgaagagcatcaggggctcgc
gccagccgaactgttcgccaggctcaaggcgcgcatgcccgacggcgaggatctcgtcgtgacccatg
gcgatgcctgcttgccgaatatcatggtggaaaatggccgcttttctggattcatcgactgtggccgg
ctgggtgtggcggaccgctatcaggacatagcgttggctacccgtgatattgctgaagagcttggcgg
cgaatgggctgaccgcttcctcgtgctttacggtatcgccgctcccgattcgcagcgcatcgccttct
atcgccttcttgacgagttcttctgagcgggactctggggttcgaaatgaccgaccaagcgacgccca
acctgccatcacgagatttcgattccaccgccgccttctatgaaaggttgggcttcggaatcgttttc
cgggacgccggctggatgatcctccagcgcggggatctcatgctggagttcttcgcccaccccaactt
gtttattgcagcttataatggttacaaataaagcaatagcatcacaaatttcacaaataaagcatttt
tttcactgcattctagttgtggtttgtccaaactcatcaatgtatcttatcatgtctgtataccgtcg
acctctagctagagcttggcgtaatcatggtcatagctgtttcctgtgtgaaattgttatccgctcac
aattccacacaacatacgagccggaagcataaagtgtaaagcctggggtgcctaatgagtgagctaac
tcacattaattgcgttgcgctcactgcccgctttccagtcgggaaacctgtcgtgccagctgcattaa
tgaatcggccaacgcgcggggagaggcggtttgcgtattgggcgctcttccgcttcctcgctcactga
ctcgctgcgctcggtcgttcggctgcggcgagcggtatcagctcactcaaaggcggtaatacggttat
ccacagaatcaggggataacgcaggaaagaacatgtgagcaaaaggccagcaaaaggccaggaaccgt
aaaaaggccgcgttgctggcgtttttccataggctccgcccccctgacgagcatcacaaaaatcgacg
ctcaagtcagaggtggcgaaacccgacaggactataaagataccaggcgtttccccctggaagctccc
tcgtgcgctctcctgttccgaccctgccgcttaccggatacctgtccgcctttctcccttcgggaagc
gtggcgctttctcatagctcacgctgtaggtatctcagttcggtgtaggtcgttcgctccaagctggg
ctgtgtgcacgaaccccccgttcagcccgaccgctgcgccttatccggtaactatcgtcttgagtcca
acccggtaagacacgacttatcgccactggcagcagccactggtaacaggattagcagagcgaggtat
gtaggcggtgctacagagttcttgaagtggtggcctaactacggctacactagaagaacagtatttgg
tatctgcgctctgctgaagccagttaccttcggaaaaagagttggtagctcttgatccggcaaacaaa
ccaccgctggtagcggtggtttttttgtttgcaagcagcagattacgcgcagaaaaaaaggatctcaa
gaagatcctttgatcttttctacggggtctgacgctcagtggaacgaaaactcacgttaagggatttt
ggtcatgagattatcaaaaaggatcttcacctagatccttttaaattaaaaatgaagttttaaatcaa
tctaaagtatatatgagtaaacttggtctgacagttaccaatgcttaatcagtgaggcacctatctca
gcgatctgtctatttcgttcatccatagttgcctgactccccgtcgtgtagataactacgatacggga
gggcttaccatctggccccagtgctgcaatgataccgcgagacccacgctcaccggctccagatttat
cagcaataaaccagccagccggaagggccgagcgcagaagtggtcctgcaactttatccgcctccatc
cagtctattaattgttgccgggaagctagagtaagtagttcgccagttaatagtttgcgcaacgttgt
tgccattgctacaggcatcgtggtgtcacgctcgtcgtttggtatggcttcattcagctccggttccc
aacgatcaaggcgagttacatgatcccccatgttgtgcaaaaaagcggttagctccttcggtcctccg
atcgttgtcagaagtaagttggccgcagtgttatcactcatggttatggcagcactgcataattctct
tactgtcatgccatccgtaagatgcttttctgtgactggtgagtactcaaccaagtcattctgagaat
agtgtatgcggcgaccgagttgctcttgcccggcgtcaatacgggataataccgcgccacatagcaga
actttaaaagtgctcatcattggaaaacgttcttcggggcgaaaactctcaaggatcttaccgctgtt
gagatccagttcgatgtaacccactcgtgcacccaactgatcttcagcatcttttactttcaccagcg
tttctgggtgagcaaaaacaggaaggcaaaatgccgcaaaaaagggaataagggcgacacggaaatgt
tgaatactcatactcttcctttttcaatattattgaagcatttatcagggttattgtctcatgagcgg
atacatatttgaatgtatttagaaaaataaacaaataggggttccgcgcacatttccccgaaaagtgc
cacctgacgtc
PVP31b pcDNA3.1 Spike Hexapro
(SEQ ID NO: 39)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATgTTTGTGTTCCTGGTGCTGCTGCCACTGGTGTCCAGCCAGTGTGTGAACCTGACCACCAG
GACCCAACTTCCTCCTGCCTACACCAACTCCTTCACCAGGGGAGTCTACTACCCTGACAAGGTGTTCA
GGTCCTCTGTGCTGCACAGCACCCAGGACCTGTTCCTGCCATTCTTCAGCAATGTGACCTGGTTCCAT
GCCATCCATGTGTCTGGCACCAATGGCACCAAGAGGTTTGACAACCCTGTGCTGCCATTCAATGATGG
AGTCTACTTTGCCAGCACAGAGAAGAGCAACATCATCAGGGGCTGGATTTTTGGCACCACCCTGGACA
GCAAGACCCAGTCCCTGCTGATTGTGAACAATGCCACCAATGTGGTGATTAAGGTGTGTGAGTTCCAG
TTCTGTAATGACCCATTCCTGGGAGTCTACTACCACAAGAACAACAAGTCCTGGATGGAGTCTGAGTT
CAGGGTCTACTCCTCTGCCAACAACTGTACCTTTGAATATGTGAGCCAACCATTCCTGATGGACTTGG
AGGGCAAGCAGGGCAACTTCAAGAACCTGAGGGAGTTTGTGTTCAAGAACATTGATGGCTACTTCAAG
ATTTACAGCAAACACACACCAATCAACCTGGTGAGGGACCTGCCACAGGGCTTCTCTGCCTTGGAACC
ACTGGTGGACCTGCCAATTGGCATCAACATCACCAGGTTCCAGACCCTGCTGGCTCTGCACAGGTCCT
ACCTGACACCTGGAGACTCCTCCTCTGGCTGGACAGCAGGAGCAGCAGCCTACTATGTGGGCTACCTC
CAACCAAGGACCTTCCTGCTGAAATACAATGAGAATGGCACCATCACAGATGCTGTGGACTGTGCCCT
GGACCCACTGTCTGAGACCAAGTGTACCCTGAAATCCTTCACAGTGGAGAAGGGCATCTACCAGACCA
GCAACTTCAGGGTCCAACCAACAGAGAGCATTGTGAGGTTTCCAAACATCACCAACCTGTGTCCATTT
GGAGAGGTGTTCAATGCCACCAGGTTTGCCTCTGTCTATGCCTGGAACAGGAAGAGGATTAGCAACTG
TGTGGCTGACTACTCTGTGCTCTACAACTCTGCCTCCTTCAGCACCTTCAAGTGTTATGGAGTGAGCC
CAACCAAACTGAATGACCTGTGTTTCACCAATGTCTATGCTGACTCCTTTGTGATTAGGGGAGATGAG
GTGAGACAGATTGCCCCTGGACAAACAGGCAAGATTGCTGACTACAACTACAAACTGCCTGATGACTT
CACAGGCTGTGTGATTGCCTGGAACAGCAACAACCTGGACAGCAAGGTGGGAGGCAACTACAACTACC
TCTACAGACTGTTCAGGAAGAGCAACCTGAAACCATTTGAGAGGGACATCAGCACAGAGATTTACCAG
GCTGGCAGCACACCATGTAATGGAGTGGAGGGCTTCAACTGTTACTTTCCACTCCAATCCTATGGCTT
CCAACCAACCAATGGAGTGGGCTACCAACCATACAGGGTGGTGGTGCTGTCCTTTGAACTGCTCCATG
CCCCTGCCACAGTGTGTGGACCAAAGAAGAGCACCAACCTGGTGAAGAACAAGTGTGTGAACTTCAAC
TTCAATGGACTGACAGGCACAGGAGTGCTGACAGAGAGCAACAAGAAGTTCCTGCCATTCCAACAGTT
TGGCAGGGACATTGCTGACACCACAGATGCTGTGAGGGACCCACAGACCTTGGAGATTCTGGACATCA
CACCATGTTCCTTTGGAGGAGTGTCTGTGATTACACCTGGCACCAACACCAGCAACCAGGTGGCTGTG
CTCTACCAGGATGTGAACTGTACTGAGGTGCCTGTGGCTATCCATGCTGACCAACTTACACCAACCTG
GAGGGTCTACAGCACAGGCAGCAATGTGTTCCAGACCAGGGCTGGCTGTCTGATTGGAGCAGAGCATG
TGAACAACTCCTATGAGTGTGACATCCCAATTGGAGCAGGCATCTGTGCCTCCTACCAGACCCAGACC
AACAGCCCAGGCTCTGCATCTTCTGTGGCAAGCCAGAGCATCATTGCCTACACAATGAGTCTGGGAGC
AGAGAACTCTGTGGCTTACAGCAACAACAGCATTGCCATCCCAACCAACTTCACCATCTCTGTGACCA
CAGAGATTCTGCCTGTGAGTATGACCAAGACCTCTGTGGACTGTACAATGTATATCTGTGGAGACAGC
ACAGAGTGTAGCAACCTGCTGCTCCAATATGGCTCCTTCTGTACCCAACTTAACAGGGCTCTGACAGG
CATTGCTGTGGAACAGGACAAGAACACCCAGGAGGTGTTTGCCCAGGTGAAGCAGATTTACAAGACAC
CTCCAATCAAGGACTTTGGAGGCTTCAACTTCAGCCAGATTCTGCCTGACCCAAGCAAGCCAAGCAAG
AGGTCCCCTATTGAGGACCTGCTGTTCAACAAGGTGACCCTGGCTGATGCTGGCTTCATCAAGCAATA
TGGAGACTGTCTGGGAGACATTGCTGCCAGGGACCTGATTTGTGCCCAGAAGTTCAATGGACTGACAG
TGCTGCCTCCACTGCTGACAGATGAGATGATTGCCCAATACACCTCTGCCCTGCTGGCTGGCACCATC
ACCTCTGGCTGGACCTTTGGAGCAGGACCAGCCCTCCAAATCCCATTTCCAATGCAGATGGCTTACAG
GTTCAATGGCATTGGAGTGACCCAGAATGTGCTCTATGAGAACCAGAAACTGATTGCCAACCAGTTCA
ACTCTGCCATTGGCAAGATTCAGGACTCCCTGTCCAGCACACCATCTGCCCTGGGCAAACTCCAAGAT
GTGGTGAACCAGAATGCCCAGGCTCTGAACACCCTGGTGAAGCAACTTTCCAGCAACTTTGGAGCCAT
CTCCTCTGTGCTGAATGACATCCTGAGCAGACTGGACCCACCAGAGGCTGAGGTCCAGATTGACAGAC
TGATTACAGGCAGACTCCAATCCCTCCAAACCTATGTGACCCAACAACTTATCAGGGCTGCTGAGATT
AGGGCATCTGCCAACCTGGCTGCCACCAAGATGAGTGAGTGTGTGCTGGGACAAAGCAAGAGGGTGGA
CTTCTGTGGCAAGGGCTACCACCTGATGAGTTTTCCACAGTCTGCCCCTCATGGAGTGGTGTTCCTGC
ATGTGACCTATGTGCCTGCCCAGGAGAAGAACTTCACCACAGCCCCTGCCATCTGCCATGATGGCAAG
GCTCACTTTCCAAGGGAGGGAGTGTTTGTGAGCAATGGCACCCACTGGTTTGTGACCCAGAGGAACTT
CTATGAACCACAGATTATCACCACAGACAACACCTTTGTGTCTGGCAACTGTGATGTGGTGATTGGCA
TTGTGAACAACACAGTCTATGACCCACTCCAACCTGAACTGGACTCCTTCAAGGAGGAACTGGACAAA
TACTTCAAGAACCACACCAGCCCTGATGTGGACCTGGGAGACATCTCTGGCATCAATGCCTCTGTGGT
GAACATCCAGAAGGAGATTGACAGACTGAATGAGGTGGCTAAGAACCTGAATGAGTCCCTGATTGACC
TCCAAGAACTGGGCAAATATGAACAATACATCAAGTGGCCATGGTACATCTGGCTGGGCTTCATTGCT
GGACTGATTGCCATTGTGATGGTGACCATAATGCTGTGTTGTATGACCTCCTGTTGTTCCTGTCTGAA
AGGCTGTTGTTCCTGTGGCTCCTGTTGTAAGTTTGATGAGGATGACTCTGAACCTGTGCTGAAAGGAG
TGAAACTGCACTACACCTGACTCGAGCTGGTACTGCATGCACGCAATGCTAGCTGCCCCTTTCCCGTC
CTGGGTACCCCGAGTCTCCCCCGACCTCGGGTCCCAGGTATGCTCCCACCTCCACCTGCCCCACTCAC
CACCTCTGCTAGTTCCAGACACCTCCCAAGCACGCAGCAATGCAGCTCAAAACGCTTAGCCTAGCCAC
ACCCCCACGGGAAACAGCAGTGATTAACCTTTAGCAATAAACGAAAGTTTAACTAAGCTATACTAACC
CCAGGGTTGGTCAATTTCGTGCCAGCCACACCCTGGAGCTAGCaaaccgctgatcagcctcgactgtg
ccttctagttgccagccatctgttgtttgcccctcccccgtgccttccttgaccctggaaggtgccac
tcccactgtcctttcctaataaaatgaggaaattgcatcgcattgtctgagtaggtgtcattctattc
tggggggtggggtggggcaggacagcaagggggaggattgggaagacaatagcaggcatgctggggat
gcggtgggctctatggcttctgaggcggaaagaaccagctggggctctagggggtatccccacgcgcc
ctgtagcggcgcattaagcgcggcgggtgtggtggttacgcgcagcgtgaccgctacacttgccagcg
ccctagcgcccgctcctttcgctttcttcccttcctttctcgccacgttcgccggctttccccgtcaa
gctctaaatcgggggctccctttagggttccgatttagtgctttacggcacctcgaccccaaaaaact
tgattagggtgatggttcacgtagtgggccatcgccctgatagacggtttttcgccctttgacgttgg
agtccacgttctttaatagtggactcttgttccaaactggaacaacactcaaccctatctcggtctat
tcttttgatttataagggattttgccgatttcggcctattggttaaaaaatgagctgatttaacaaaa
atttaacgcgaattaattctgtggaatgtgtgtcagttagggtgtggaaagtccccaggctccccagc
aggcagaagtatgcaaagcatgcatctcaattagtcagcaaccaggtgtggaaagtccccaggctccc
cagcaggcagaagtatgcaaagcatgcatctcaattagtcagcaaccatagtcccgcccctaactccg
cccatcccgcccctaactccgcccagttccgcccattctccgccccatggctgactaattttttttat
ttatgcagaggccgaggccgcctctgcctctgagctattccagaagtagtgaggaggcttttttggag
gcctaggcttttgcaaaaagctcccgggagcttgtatatccattttcggatctgatcaagagacagga
tgaggatcgtttcgcatgattgaacaagatggattgcacgcaggttctccggccgcttgggtggagag
gctattcggctatgactgggcacaacagacaatcggctgctctgatgccgccgtgttccggctgtcag
cgcaggggcgcccggttctttttgtcaagaccgacctgtccggtgccctgaatgaactgcaggacgag
gcagcgcggctatcgtggctggccacgacgggcgttccttgcgcagctgtgctcgacgttgtcactga
agcgggaagggactggctgctattgggcgaagtgccggggcaggatctcctgtcatctcaccttgctc
ctgccgagaaagtatccatcatggctgatgcaatgcggcggctgcatacgcttgatccggctacctgc
ccattcgaccaccaagcgaaacatcgcatcgagcgagcacgtactcggatggaagccggtcttgtcga
tcaggatgatctggacgaagagcatcaggggctcgcgccagccgaactgttcgccaggctcaaggcgc
gcatgcccgacggcgaggatctcgtcgtgacccatggcgatgcctgcttgccgaatatcatggtggaa
aatggccgcttttctggattcatcgactgtggccggctgggtgtggcggaccgctatcaggacatagc
gttggctacccgtgatattgctgaagagcttggcggcgaatgggctgaccgcttcctcgtgctttacg
gtatcgccgctcccgattcgcagcgcatcgccttctatcgccttcttgacgagttcttctgagcggga
ctctggggttcgaaatgaccgaccaagcgacgcccaacctgccatcacgagatttcgattccaccgcc
gccttctatgaaaggttgggcttcggaatcgttttccgggacgccggctggatgatcctccagcgcgg
ggatctcatgctggagttcttcgcccaccccaacttgtttattgcagcttataatggttacaaataaa
gcaatagcatcacaaatttcacaaataaagcatttttttcactgcattctagttgtggtttgtccaaa
ctcatcaatgtatcttatcatgtctgtataccgtcgacctctagctagagcttggcgtaatcatggtc
atagctgtttcctgtgtgaaattgttatccgctcacaattccacacaacatacgagccggaagcataa
agtgtaaagcctggggtgcctaatgagtgagctaactcacattaattgcgttgcgctcactgcccgct
ttccagtcgggaaacctgtcgtgccagctgcattaatgaatcggccaacgcgcggggagaggcggttt
gcgtattgggcgctcttccgcttcctcgctcactgactcgctgcgctcggtcgttcggctgcggcgag
cggtatcagctcactcaaaggcggtaatacggttatccacagaatcaggggataacgcaggaaagaac
atgtgagcaaaaggccagcaaaaggccaggaaccgtaaaaaggccgcgttgctggcgtttttccatag
gctccgcccccctgacgagcatcacaaaaatcgacgctcaagtcagaggtggcgaaacccgacaggac
tataaagataccaggcgtttccccctggaagctccctcgtgcgctctcctgttccgaccctgccgctt
accggatacctgtccgcctttctcccttcgggaagcgtggcgctttctcatagctcacgctgtaggta
tctcagttcggtgtaggtcgttcgctccaagctgggctgtgtgcacgaaccccccgttcagcccgacc
gctgcgccttatccggtaactatcgtcttgagtccaacccggtaagacacgacttatcgccactggca
gcagccactggtaacaggattagcagagcgaggtatgtaggcggtgctacagagttcttgaagtggtg
gcctaactacggctacactagaagaacagtatttggtatctgcgctctgctgaagccagttaccttcg
gaaaaagagttggtagctcttgatccggcaaacaaaccaccgctggtagcggtggtttttttgtttgc
aagcagcagattacgcgcagaaaaaaaggatctcaagaagatcctttgatcttttctacggggtctga
cgctcagtggaacgaaaactcacgttaagggattttggtcatgagattatcaaaaaggatcttcacct
agatccttttaaattaaaaatgaagttttaaatcaatctaaagtatatatgagtaaacttggtctgac
agttaccaatgcttaatcagtgaggcacctatctcagcgatctgtctatttcgttcatccatagttgc
ctgactccccgtcgtgtagataactacgatacgggagggcttaccatctggccccagtgctgcaatga
taccgcgagacccacgctcaccggctccagatttatcagcaataaaccagccagccggaagggccgag
cgcagaagtggtcctgcaactttatccgcctccatccagtctattaattgttgccgggaagctagagt
aagtagttcgccagttaatagtttgcgcaacgttgttgccattgctacaggcatcgtggtgtcacgct
cgtcgtttggtatggcttcattcagctccggttcccaacgatcaaggcgagttacatgatcccccatg
ttgtgcaaaaaagcggttagctccttcggtcctccgatcgttgtcagaagtaagttggccgcagtgtt
atcactcatggttatggcagcactgcataattctcttactgtcatgccatccgtaagatgcttttctg
tgactggtgagtactcaaccaagtcattctgagaatagtgtatgcggcgaccgagttgctcttgcccg
gcgtcaatacgggataataccgcgccacatagcagaactttaaaagtgctcatcattggaaaacgttc
ttcggggcgaaaactctcaaggatcttaccgctgttgagatccagttcgatgtaacccactcgtgcac
ccaactgatcttcagcatcttttactttcaccagcgtttctgggtgagcaaaaacaggaaggcaaaat
gccgcaaaaaagggaataagggcgacacggaaatgttgaatactcatactcttcctttttcaatatta
ttgaagcatttatcagggttattgtctcatgagcggatacatatttgaatgtatttagaaaaataaac
aaataggggttccgcgcacatttccccgaaaagtgccacctgacgtc
PVP33b SARSCOV UTR Hexapro
(SEQ ID NO: 40)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATgTTCATCTTCCTGCTGTTCCTGACCCTGACCTCTGGCTCTGACCTGGACAGGTGTACCAC
CTTTGATGATGTCCAGGCTCCAAACTACACCCAACACACCTCCAGTATGAGGGGAGTCTACTACCCTG
ATGAGATTTTCAGGTCTGACACCCTCTACCTGACCCAGGACCTGTTCCTGCCATTCTACAGCAATGTG
ACAGGCTTCCACACCATCAACCACACCTTTGACAACCCTGTGATTCCATTCAAGGATGGCATCTACTT
TGCTGCCACAGAGAAGAGCAATGTGGTGAGGGGCTGGGTGTTTGGCAGCACAATGAACAACAAGAGCC
AGTCTGTGATTATCATCAACAACAGCACCAATGTGGTGATTAGGGCTTGTAACTTTGAACTGTGTGAC
AACCCATTCTTTGCTGTGAGCAAGCCTATGGGCACCCAGACCCACACAATGATTTTTGACAATGCCTT
CAACTGTACCTTTGAATACATCTCTGATGCCTTCTCCCTGGATGTGTCTGAGAAGTCTGGCAACTTCA
AACACCTGAGGGAGTTTGTGTTCAAGAACAAGGATGGCTTCCTCTATGTCTACAAGGGCTACCAACCA
ATTGATGTGGTGAGGGACCTGCCATCTGGCTTCAACACCCTGAAACCAATCTTCAAACTGCCACTGGG
CATCAACATCACCAACTTCAGGGCTATCCTGACAGCCTTCAGCCCTGCCCAGGACACCTGGGGCACCT
CTGCTGCTGCCTACTTTGTGGGCTACCTGAAACCAACCACCTTTATGCTGAAATATGATGAGAATGGC
ACCATCACAGATGCTGTGGACTGTAGCCAGAACCCACTGGCTGAACTGAAATGTTCTGTGAAGTCCTT
TGAGATTGACAAGGGCATCTACCAGACCAGCAACTTCAGGGTGGTGCCATCTGGAGATGTGGTGAGGT
TTCCAAACATCACCAACCTGTGTCCATTTGGAGAGGTGTTCAATGCCACCAAGTTTCCATCTGTCTAT
GCCTGGGAGAGGAAGAAGATTAGCAACTGTGTGGCTGACTACTCTGTGCTCTACAACAGCACCTTCTT
CAGCACCTTCAAGTGTTATGGAGTGTCTGCCACCAAACTGAATGACCTGTGTTTCAGCAATGTCTATG
CTGACTCCTTTGTGGTGAAGGGAGATGATGTGAGACAGATTGCCCCTGGACAAACAGGAGTGATTGCT
GACTACAACTACAAACTGCCTGATGACTTTATGGGCTGTGTGCTGGCTTGGAACACCAGGAACATTGA
TGCCACCAGCACAGGCAACTACAACTACAAATACAGATACCTGAGACATGGCAAACTGAGACCATTTG
AGAGGGACATCAGCAATGTGCCATTCAGCCCTGATGGCAAGCCATGTACTCCTCCTGCCCTGAACTGT
TACTGGCCACTGAATGACTATGGCTTCTACACCACCACAGGCATTGGCTACCAACCATACAGGGTGGT
GGTGCTGTCCTTTGAACTGCTGAATGCCCCTGCCACAGTGTGTGGACCAAAACTGAGCACAGACCTGA
TTAAGAACCAGTGTGTGAACTTCAACTTCAATGGACTGACAGGCACAGGAGTGCTGACACCATCCAGC
AAGAGGTTCCAACCATTCCAACAGTTTGGCAGGGATGTGTCTGACTTCACAGACTCTGTGAGGGACCC
AAAGACCTCTGAGATTCTGGACATCAGCCCATGTTCCTTTGGAGGAGTGTCTGTGATTACACCTGGCA
CCAATGCCTCCTCTGAGGTGGCTGTGCTCTACCAGGATGTGAACTGTACTGATGTGAGCACAGCCATC
CATGCTGACCAACTTACACCTGCCTGGAGGATTTACAGCACAGGCAACAATGTGTTCCAGACCCAGGC
TGGCTGTCTGATTGGAGCAGAGCATGTGGACACCTCCTATGAGTGTGACATCCCAATTGGAGCAGGCA
TCTGTGCCTCCTACCACACAGTGTCCCTGCTGAGGAGCACCAGCCAGAAGAGCATTGTGGCTTACACA
ATGAGTCTGGGAGCAGACTCCAGCATTGCCTACAGCAACAACACCATTGCCATCCCAACCAACTTCAG
CATCAGCATCACCACAGAGGTGATGCCTGTGAGTATGGCTAAGACCTCTGTGGACTGTAATATGTATA
TCTGTGGAGACAGCACAGAGTGTGCCAACCTGCTGCTCCAATATGGCTCCTTCTGTACCCAACTTAAC
AGGGCTCTGTCTGGCATTGCTGCTGAACAGGACAGGAACACCAGGGAGGTGTTTGCCCAGGTGAAGCA
GATGTATAAGACACCAACCCTGAAATACTTTGGAGGCTTCAACTTCAGCCAGATTCTGCCTGACCCAC
TGAAACCAACCAAGAGGTCCCCAATTGAGGACCTGCTGTTCAACAAGGTGACCCTGGCTGATGCTGGC
TTTATGAAGCAATATGGAGAGTGTCTGGGAGACATCAATGCCAGGGACCTGATTTGTGCCCAGAAGTT
CAATGGACTGACAGTGCTGCCTCCACTGCTGACAGATGATATGATTGCTGCCTACACAGCAGCCCTGG
TGTCTGGCACAGCCACAGCAGGCTGGACCTTTGGAGCAGGACCAGCCCTCCAAATCCCATTTCCAATG
CAGATGGCTTACAGGTTCAATGGCATTGGAGTGACCCAGAATGTGCTCTATGAGAACCAGAAGCAGAT
TGCCAACCAGTTCAACAAGGCTATCAGCCAGATTCAGGAGTCCCTGACCACCACCCCAACAGCCCTGG
GCAAACTCCAAGATGTGGTGAACCAGAATGCCCAGGCTCTGAACACCCTGGTGAAGCAACTTTCCAGC
AACTTTGGAGCCATCTCCTCTGTGCTGAATGACATCCTGAGCAGACTGGACCCACCAGAGGCTGAGGT
CCAGATTGACAGACTGATTACAGGCAGACTCCAATCCCTCCAAACCTATGTGACCCAACAACTTATCA
GGGCTGCTGAGATTAGGGCATCTGCCAACCTGGCTGCCACCAAGATGAGTGAGTGTGTGCTGGGACAA
AGCAAGAGGGTGGACTTCTGTGGCAAGGGCTACCACCTGATGAGTTTTCCACAGGCTGCCCCTCATGG
AGTGGTGTTCCTGCATGTGACCTATGTGCCAAGCCAGGAGAGGAACTTCACCACAGCCCCTGCCATCT
GCCATGAGGGCAAGGCTTACTTTCCAAGGGAGGGAGTGTTTGTGTTCAATGGCACCTCCTGGTTCATC
ACCCAGAGGAACTTCTTCAGCCCACAGATTATCACCACAGACAACACCTTTGTGTCTGGCAACTGTGA
TGTGGTGATTGGCATCATCAACAACACAGTCTATGACCCACTCCAACCTGAACTGGACTCCTTCAAGG
AGGAACTGGACAAATACTTCAAGAACCACACCAGCCCTGATGTGGACCTGGGAGACATCTCTGGCATC
AATGCCTCTGTGGTGAACATCCAGAAGGAGATTGACAGACTGAATGAGGTGGCTAAGAACCTGAATGA
GTCCCTGATTGACCTCCAAGAACTGGGCAAATATGAACAATACATCAAGTGGCCATGGTATGTGTGGC
TGGGCTTCATTGCTGGACTGATTGCCATTGTGATGGTGACCATCCTGCTGTGTTGTATGACCTCCTGT
TGTTCCTGTCTGAAAGGAGCCTGTTCCTGTGGCTCCTGTTGTAAGTTTGATGAGGATGACTCTGAACC
TGTGCTGAAAGGAGTGAAACTGCACTACACCTAACTCGAGCTGGTACTGCATGCACGCAATGCTAGCT
GCCCCTTTCCCGTCCTGGGTACCCCGAGTCTCCCCCGACCTCGGGTCCCAGGTATGCTCCCACCTCCA
CCTGCCCCACTCACCACCTCTGCTAGTTCCAGACACCTCCCAAGCACGCAGCAATGCAGCTCAAAACG
CTTAGCCTAGCCACACCCCCACGGGAAACAGCAGTGATTAACCTTTAGCAATAAACGAAAGTTTAACT
AAGCTATACTAACCCCAGGGTTGGTCAATTTCGTGCCAGCCACACCCTGGAGCTAGCaaaccgctgat
cagcctcgactgtgccttctagttgccagccatctgttgtttgcccctcccccgtgccttccttgacc
ctggaaggtgccactcccactgtcctttcctaataaaatgaggaaattgcatcgcattgtctgagtag
gtgtcattctattctggggggtggggtggggcaggacagcaagggggaggattgggaagacaatagca
ggcatgctggggatgcggtgggctctatggcttctgaggcggaaagaaccagctggggctctaggggg
tatccccacgcgccctgtagcggcgcattaagcgcggcgggtgtggtggttacgcgcagcgtgaccgc
tacacttgccagcgccctagcgcccgctcctttcgctttcttcccttcctttctcgccacgttcgccg
gctttccccgtcaagctctaaatcgggggctccctttagggttccgatttagtgctttacggcacctc
gaccccaaaaaacttgattagggtgatggttcacgtagtgggccatcgccctgatagacggtttttcg
ccctttgacgttggagtccacgttctttaatagtggactcttgttccaaactggaacaacactcaacc
ctatctcggtctattcttttgatttataagggattttgccgatttcggcctattggttaaaaaatgag
ctgatttaacaaaaatttaacgcgaattaattctgtggaatgtgtgtcagttagggtgtggaaagtcc
ccaggctccccagcaggcagaagtatgcaaagcatgcatctcaattagtcagcaaccaggtgtggaaa
gtccccaggctccccagcaggcagaagtatgcaaagcatgcatctcaattagtcagcaaccatagtcc
cgcccctaactccgcccatcccgcccctaactccgcccagttccgcccattctccgccccatggctga
ctaattttttttatttatgcagaggccgaggccgcctctgcctctgagctattccagaagtagtgagg
aggcttttttggaggcctaggcttttgcaaaaagctcccgggagcttgtatatccattttcggatctg
atcaagagacaggatgaggatcgtttcgcatgattgaacaagatggattgcacgcaggttctccggcc
gcttgggtggagaggctattcggctatgactgggcacaacagacaatcggctgctctgatgccgccgt
gttccggctgtcagcgcaggggcgcccggttctttttgtcaagaccgacctgtccggtgccctgaatg
aactgcaggacgaggcagcgcggctatcgtggctggccacgacgggcgttccttgcgcagctgtgctc
gacgttgtcactgaagcgggaagggactggctgctattgggcgaagtgccggggcaggatctcctgtc
atctcaccttgctcctgccgagaaagtatccatcatggctgatgcaatgcggcggctgcatacgcttg
atccggctacctgcccattcgaccaccaagcgaaacatcgcatcgagcgagcacgtactcggatggaa
gccggtcttgtcgatcaggatgatctggacgaagagcatcaggggctcgcgccagccgaactgttcgc
caggctcaaggcgcgcatgcccgacggcgaggatctcgtcgtgacccatggcgatgcctgcttgccga
atatcatggtggaaaatggccgcttttctggattcatcgactgtggccggctgggtgtggcggaccgc
tatcaggacatagcgttggctacccgtgatattgctgaagagcttggcggcgaatgggctgaccgctt
cctcgtgctttacggtatcgccgctcccgattcgcagcgcatcgccttctatcgccttcttgacgagt
tcttctgagcgggactctggggttcgaaatgaccgaccaagcgacgcccaacctgccatcacgagatt
tcgattccaccgccgccttctatgaaaggttgggcttcggaatcgttttccgggacgccggctggatg
atcctccagcgcggggatctcatgctggagttcttcgcccaccccaacttgtttattgcagcttataa
tggttacaaataaagcaatagcatcacaaatttcacaaataaagcatttttttcactgcattctagtt
gtggtttgtccaaactcatcaatgtatcttatcatgtctgtataccgtcgacctctagctagagcttg
gcgtaatcatggtcatagctgtttcctgtgtgaaattgttatccgctcacaattccacacaacatacg
agccggaagcataaagtgtaaagcctggggtgcctaatgagtgagctaactcacattaattgcgttgc
gctcactgcccgctttccagtcgggaaacctgtcgtgccagctgcattaatgaatcggccaacgcgcg
gggagaggcggtttgcgtattgggcgctcttccgcttcctcgctcactgactcgctgcgctcggtcgt
tcggctgcggcgagcggtatcagctcactcaaaggcggtaatacggttatccacagaatcaggggata
acgcaggaaagaacatgtgagcaaaaggccagcaaaaggccaggaaccgtaaaaaggccgcgttgctg
gcgtttttccataggctccgcccccctgacgagcatcacaaaaatcgacgctcaagtcagaggtggcg
aaacccgacaggactataaagataccaggcgtttccccctggaagctccctcgtgcgctctcctgttc
cgaccctgccgcttaccggatacctgtccgcctttctcccttcgggaagcgtggcgctttctcatagc
tcacgctgtaggtatctcagttcggtgtaggtcgttcgctccaagctgggctgtgtgcacgaaccccc
cgttcagcccgaccgctgcgccttatccggtaactatcgtcttgagtccaacccggtaagacacgact
tatcgccactggcagcagccactggtaacaggattagcagagcgaggtatgtaggcggtgctacagag
ttcttgaagtggtggcctaactacggctacactagaagaacagtatttggtatctgcgctctgctgaa
gccagttaccttcggaaaaagagttggtagctcttgatccggcaaacaaaccaccgctggtagcggtg
gtttttttgtttgcaagcagcagattacgcgcagaaaaaaaggatctcaagaagatcctttgatcttt
tctacggggtctgacgctcagtggaacgaaaactcacgttaagggattttggtcatgagattatcaaa
aaggatcttcacctagatccttttaaattaaaaatgaagttttaaatcaatctaaagtatatatgagt
aaacttggtctgacagttaccaatgcttaatcagtgaggcacctatctcagcgatctgtctatttcgt
tcatccatagttgcctgactccccgtcgtgtagataactacgatacgggagggcttaccatctggccc
cagtgctgcaatgataccgcgagacccacgctcaccggctccagatttatcagcaataaaccagccag
ccggaagggccgagcgcagaagtggtcctgcaactttatccgcctccatccagtctattaattgttgc
cgggaagctagagtaagtagttcgccagttaatagtttgcgcaacgttgttgccattgctacaggcat
cgtggtgtcacgctcgtcgtttggtatggcttcattcagctccggttcccaacgatcaaggcgagtta
catgatcccccatgttgtgcaaaaaagcggttagctccttcggtcctccgatcgttgtcagaagtaag
ttggccgcagtgttatcactcatggttatggcagcactgcataattctcttactgtcatgccatccgt
aagatgcttttctgtgactggtgagtactcaaccaagtcattctgagaatagtgtatgcggcgaccga
gttgctcttgcccggcgtcaatacgggataataccgcgccacatagcagaactttaaaagtgctcatc
attggaaaacgttcttcggggcgaaaactctcaaggatcttaccgctgttgagatccagttcgatgta
acccactcgtgcacccaactgatcttcagcatcttttactttcaccagcgtttctgggtgagcaaaaa
caggaaggcaaaatgccgcaaaaaagggaataagggcgacacggaaatgttgaatactcatactcttc
ctttttcaatattattgaagcatttatcagggttattgtctcatgagcggatacatatttgaatgtat
ttagaaaaataaacaaataggggttccgcgcacatttccccgaaaagtgccacctgacgtc
PVP34c MERSCOV UTR Hexapro
(SEQ ID NO: 41)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATgATTCACTCTGTGTTCCTGCTGATGTTCCTGCTGACACCAACAGAGTCCTATGTGGATGT
GGGACCTGACTCTGTGAAGTCTGCCTGTATTGAGGTGGACATCCAACAGACCTTCTTTGACAAGACCT
GGCCAAGACCAATTGATGTGAGCAAGGCTGATGGCATCATCTACCCACAGGGCAGGACCTACAGCAAC
ATCACCATCACCTACCAGGGACTGTTTCCATACCAGGGAGATCATGGAGATATGTATGTCTACTCTGC
TGGTCATGCCACAGGCACCACACCACAGAAACTGTTTGTGGCTAACTACAGCCAGGATGTGAAGCAGT
TTGCCAATGGCTTTGTGGTGAGGATTGGAGCAGCAGCCAACAGCACAGGCACAGTGATTATCAGCCCA
AGCACCTCTGCCACCATCAGGAAGATTTACCCTGCCTTTATGCTGGGCTCCTCTGTGGGCAACTTCTC
TGATGGCAAGATGGGCAGGTTCTTCAACCACACCCTGGTGCTGCTGCCTGATGGCTGTGGCACCCTGC
TGAGGGCTTTCTACTGTATCTTGGAACCAAGGTCTGGCAACCACTGTCCTGCTGGCAACTCCTACACC
TCCTTTGCCACCTACCACACACCTGCCACAGACTGTTCTGATGGCAACTACAACAGGAATGCCTCCCT
GAACTCCTTCAAGGAATACTTCAACCTGAGGAACTGTACCTTTATGTACACCTACAACATCACAGAGG
ATGAGATTTTGGAGTGGTTTGGCATCACCCAGACAGCCCAGGGAGTGCATCTGTTCTCGAGCAGATAT
GTGGACCTCTATGGAGGCAATATGTTCCAGTTTGCCACCCTGCCTGTCTATGACACCATCAAATACTA
CAGCATCATCCCACACAGCATCAGGAGCATCCAGTCTGACAGGAAGGCTTGGGCTGCCTTCTATGTCT
ACAAACTCCAACCACTGACCTTCCTGCTGGACTTCTCTGTGGATGGCTACATCAGGAGGGCTATTGAC
TGTGGCTTCAATGACCTGAGCCAACTTCACTGTTCCTATGAGTCCTTTGATGTGGAGTCTGGAGTCTA
CTCTGTGTCCTCCTTTGAGGCTAAGCCATCTGGCTCTGTGGTGGAACAGGCTGAGGGAGTGGAGTGTG
ACTTCAGCCCACTGCTGTCTGGCACACCTCCACAGGTCTACAACTTCAAGAGACTGGTGTTCACCAAC
TGTAACTACAACCTGACCAAACTGCTGTCCCTGTTCTCTGTGAATGACTTCACTTGTAGCCAGATTAG
CCCTGCTGCCATTGCCAGCAACTGTTACTCCTCCCTGATTCTGGACTACTTCTCCTACCCACTGAGTA
TGAAGTCTGACCTGTCTGTGTCCTCTGCTGGACCAATCAGCCAGTTCAACTACAAGCAGTCCTTCAGC
AACCCAACTTGTCTGATTCTGGCTACAGTGCCACACAACCTGACCACCATCACCAAGCCACTGAAATA
CTCCTACATCAACAAGTGTAGCAGACTGCTGTCTGATGACAGGACAGAGGTGCCACAACTAGTGAATG
CCAACCAATACAGCCCATGTGTGAGCATTGTGCCAAGCACAGTGTGGGAGGATGGAGACTACTACAGG
AAGCAACTTAGCCCATTGGAGGGAGGAGGCTGGCTGGTGGCATCTGGCAGCACAGTGGCTATGACAGA
ACAACTCCAAATGGGCTTTGGCATCACAGTCCAATATGGCACAGACACCAACTCTGTGTGTCCAAAAT
TGGAGTTTGCCAATGACACCAAGATTGCCAGCCAACTTGGCAACTGTGTGGAATACTCCCTCTATGGA
GTGTCTGGCAGGGGAGTGTTCCAGAACTGTACTGCTGTGGGAGTGAGACAACAGAGGTTTGTCTATGA
TGCCTACCAGAACCTGGTGGGCTACTACTCTGATGATGGCAACTACTACTGTCTGAGGGCTTGTGTGT
CTGTGCCTGTGTCTGTGATTTATGACAAGGAGACCAAGACCCATGCCACCCTGTTTGGCTCTGTGGCT
TGTGAACACATCTCCAGCACAATGAGTCAATACAGCAGGAGCACCAGGAGTATGCTGAAAAGGAGGGA
CAGCACATATGGACCACTCCAAACACCTGTGGGCTGTGTGCTGGGACTGGTGAACTCCTCCCTGTTTG
TGGAGGACTGTAAACTGCCACTGGGACAATCCCTGTGTGCCCTGCCTGACACACCAAGCACCCTGACA
CCAAGGTCTGTGAGGTCTGTGCCTGGAGAGATGAGACTGGCAAGCATTGCCTTCAACCACCCAATCCA
GGTGGACCAACTTAACTCCTCCTACTTCAAACTGAGCATCCCAACCAACTTCTCCTTTGGAGTGACCC
AGGAATACATCCAGACCACCATCCAGAAGGTGACAGTGGACTGTAAGCAATATGTGTGTAATGGCTTC
CAGAAGTGTGAACAACTTCTGAGGGAATATGGACAATTCTGTAGCAAGATAAACCAGGCTCTTCATGG
AGCCAACCTGAGACAGGATGACTCTGTGAGGAACCTGTTTGCCTCTGTGAAGTCCAGCCAGTCCAGCC
CAATCATCCCTGGCTTTGGAGGAGACTTCAACCTGACCCTGTTGGAACCGGTGAGCATCAGCACAGGC
AGCAGGTCTGCCAGGTCTCCAATTGAGGACCTGCTGTTTGACAAGGTGACCATTGCTGACCCTGGCTA
TATGCAGGGCTATGATGACTGTATGCAACAGGGACCTGCCTCTGCCAGGGACCTGATTTGTGCCCAAT
ATGTGGCTGGCTACAAGGTGCTGCCTCCACTGATGGATGTGAATATGGAGGCTGCCTACACCTCCTCC
CTGCTGGGCAGCATTGCTGGAGTGGGCTGGACTGCAGGACTGTCCCCATTTGCTGCCATCCCATTTCC
ACAGAGCATCTTCTACAGACTGAATGGAGTGGGCATCACCCAACAGGTGCTGTCTGAGAACCAGAAAC
TGATTGCCAACAAGTTCAACCAGGCTCTGGGAGCTATGCAGACAGGCTTCACCACCACCCCAGAGGCT
TTCCAGAAGGTCCAGGATGCTGTGAACAACAATGCCCAGGCTCTGAGCAAACTGGCATCTGAACTGAG
CAACACCTTTGGAGCCATCTCTGCTAGCATTGGAGACATCATCCAGAGACTGGATCCACCAGAACAGG
ATGCCCAGATTGACAGACTGATAAATGGCAGACTGACCACCCTGAATGCCTTTGTGGCTCAACAACTT
GTGAGGTCTGAGTCTGCTGCCCTGTCTGCCCAACTTGCCAAGGACAAGGTGAATGAGTGTGTGAAGGC
TCAAAGCAAGAGGTCTGGCTTCTGTGGACAAGGCACCCACATTGTGTCCTTTGTGGTGAATGCCCCAA
ATGGACTCTACTTTATGCATGTGGGCTACTACCCAAGCAACCACATTGAGGTGGTGTCTGCCTATGGA
CTGTGTGATGCTGCCAACCCAACCAACTGTATTGCCCCTGTGAATGGCTACTTCATCAAGACCAACAA
CACCAGGATTGTGGATGAGTGGTCCTACACAGGCTCCTCCTTCTATGCCCCTGAACCAATCACCTCCC
TGAACACCAAATATGTGGCTCCACAGGTGACCTACCAGAACATCAGCACCAACCTGCCTCCTCCACTG
CTGGGCAACAGCACAGGCATTGACTTCCAGGATGAACTGGATGAGTTCTTCAAGAATGTGAGCACCAG
CATCCCAAACTTTGGCTCCCTGACCCAGATAAACACCACCCTGCTGGACCTGACCTATGAGATGCTGT
CCCTCCAACAGGTGGTGAAGGCTCTGAATGAGTCCTACATTGACCTGAAAGAACTGGGCAACTACACC
TACTACAACAAGTGGCCATGGTACATCTGGCTGGGCTTCATCGCTGGCCTGGTGGCCCTGGCGCTGTG
CGTGTTCTTCATCCTGTGCTGCACCGGCTGCGGCACCAACTGCATGGGCAAGCTGAAGTGCAACAGGT
GCTGCGACAGGTACGAGGAGTACGACCTGGAGCCCCACAAGGTGCACGTACATTAACTCGAGCTGGTA
CTGCATGCACGCAATGCTAGCTGCCCCTTTCCCGTCCTGGGTACCCCGAGTCTCCCCCGACCTCGGGT
CCCAGGTATGCTCCCACCTCCACCTGCCCCACTCACCACCTCTGCTAGTTCCAGACACCTCCCAAGCA
CGCAGCAATGCAGCTCAAAACGCTTAGCCTAGCCACACCCCCACGGGAAACAGCAGTGATTAACCTTT
AGCAATAAACGAAAGTTTAACTAAGCTATACTAACCCCAGGGTTGGTCAATTTCGTGCCAGCCACACC
CTGGAGCTAGCaaaccgctgatcagcctcgactgtgccttctagttgccagccatctgttgtttgccc
ctcccccgtgccttccttgaccctggaaggtgccactcccactgtcctttcctaataaaatgaggaaa
ttgcatcgcattgtctgagtaggtgtcattctattctggggggtggggtggggcaggacagcaagggg
gaggattgggaagacaatagcaggcatgctggggatgcggtgggctctatggcttctgaggcggaaag
aaccagctggggctctagggggtatccccacgcgccctgtagcggcgcattaagcgcggcgggtgtgg
tggttacgcgcagcgtgaccgctacacttgccagcgccctagcgcccgctcctttcgctttcttccct
tcctttctcgccacgttcgccggctttccccgtcaagctctaaatcgggggctccctttagggttccg
atttagtgctttacggcacctcgaccccaaaaaacttgattagggtgatggttcacgtagtgggccat
cgccctgatagacggtttttcgccctttgacgttggagtccacgttctttaatagtggactcttgttc
caaactggaacaacactcaaccctatctcggtctattcttttgatttataagggattttgccgatttc
ggcctattggttaaaaaatgagctgatttaacaaaaatttaacgcgaattaattctgtggaatgtgtg
tcagttagggtgtggaaagtccccaggctccccagcaggcagaagtatgcaaagcatgcatctcaatt
agtcagcaaccaggtgtggaaagtccccaggctccccagcaggcagaagtatgcaaagcatgcatctc
aattagtcagcaaccatagtcccgcccctaactccgcccatcccgcccctaactccgcccagttccgc
ccattctccgccccatggctgactaattttttttatttatgcagaggccgaggccgcctctgcctctg
agctattccagaagtagtgaggaggcttttttggaggcctaggcttttgcaaaaagctcccgggagct
tgtatatccattttcggatctgatcaagagacaggatgaggatcgtttcgcatgattgaacaagatgg
attgcacgcaggttctccggccgcttgggtggagaggctattcggctatgactgggcacaacagacaa
tcggctgctctgatgccgccgtgttccggctgtcagcgcaggggcgcccggttctttttgtcaagacc
gacctgtccggtgccctgaatgaactgcaggacgaggcagcgcggctatcgtggctggccacgacggg
cgttccttgcgcagctgtgctcgacgttgtcactgaagcgggaagggactggctgctattgggcgaag
tgccggggcaggatctcctgtcatctcaccttgctcctgccgagaaagtatccatcatggctgatgca
atgcggcggctgcatacgcttgatccggctacctgcccattcgaccaccaagcgaaacatcgcatcga
gcgagcacgtactcggatggaagccggtcttgtcgatcaggatgatctggacgaagagcatcaggggc
tcgcgccagccgaactgttcgccaggctcaaggcgcgcatgcccgacggcgaggatctcgtcgtgacc
catggcgatgcctgcttgccgaatatcatggtggaaaatggccgcttttctggattcatcgactgtgg
ccggctgggtgtggcggaccgctatcaggacatagcgttggctacccgtgatattgctgaagagcttg
gcggcgaatgggctgaccgcttcctcgtgctttacggtatcgccgctcccgattcgcagcgcatcgcc
ttctatcgccttcttgacgagttcttctgagcgggactctggggttcgaaatgaccgaccaagcgacg
cccaacctgccatcacgagatttcgattccaccgccgccttctatgaaaggttgggcttcggaatcgt
tttccgggacgccggctggatgatcctccagcgcggggatctcatgctggagttcttcgcccacccca
acttgtttattgcagcttataatggttacaaataaagcaatagcatcacaaatttcacaaataaagca
tttttttcactgcattctagttgtggtttgtccaaactcatcaatgtatcttatcatgtctgtatacc
gtcgacctctagctagagcttggcgtaatcatggtcatagctgtttcctgtgtgaaattgttatccgc
tcacaattccacacaacatacgagccggaagcataaagtgtaaagcctggggtgcctaatgagtgagc
taactcacattaattgcgttgcgctcactgcccgctttccagtcgggaaacctgtcgtgccagctgca
ttaatgaatcggccaacgcgcggggagaggcggtttgcgtattgggcgctcttccgcttcctcgctca
ctgactcgctgcgctcggtcgttcggctgcggcgagcggtatcagctcactcaaaggcggtaatacgg
ttatccacagaatcaggggataacgcaggaaagaacatgtgagcaaaaggccagcaaaaggccaggaa
ccgtaaaaaggccgcgttgctggcgtttttccataggctccgcccccctgacgagcatcacaaaaatc
gacgctcaagtcagaggtggcgaaacccgacaggactataaagataccaggcgtttccccctggaagc
tccctcgtgcgctctcctgttccgaccctgccgcttaccggatacctgtccgcctttctcccttcggg
aagcgtggcgctttctcatagctcacgctgtaggtatctcagttcggtgtaggtcgttcgctccaagc
tgggctgtgtgcacgaaccccccgttcagcccgaccgctgcgccttatccggtaactatcgtcttgag
tccaacccggtaagacacgacttatcgccactggcagcagccactggtaacaggattagcagagcgag
gtatgtaggcggtgctacagagttcttgaagtggtggcctaactacggctacactagaagaacagtat
ttggtatctgcgctctgctgaagccagttaccttcggaaaaagagttggtagctcttgatccggcaaa
caaaccaccgctggtagcggtggtttttttgtttgcaagcagcagattacgcgcagaaaaaaaggatc
tcaagaagatcctttgatcttttctacggggtctgacgctcagtggaacgaaaactcacgttaaggga
ttttggtcatgagattatcaaaaaggatcttcacctagatccttttaaattaaaaatgaagttttaaa
tcaatctaaagtatatatgagtaaacttggtctgacagttaccaatgcttaatcagtgaggcacctat
ctcagcgatctgtctatttcgttcatccatagttgcctgactccccgtcgtgtagataactacgatac
gggagggcttaccatctggccccagtgctgcaatgataccgcgagacccacgctcaccggctccagat
ttatcagcaataaaccagccagccggaagggccgagcgcagaagtggtcctgcaactttatccgcctc
catccagtctattaattgttgccgggaagctagagtaagtagttcgccagttaatagtttgcgcaacg
ttgttgccattgctacaggcatcgtggtgtcacgctcgtcgtttggtatggcttcattcagctccggt
tcccaacgatcaaggcgagttacatgatcccccatgttgtgcaaaaaagcggttagctccttcggtcc
tccgatcgttgtcagaagtaagttggccgcagtgttatcactcatggttatggcagcactgcataatt
ctcttactgtcatgccatccgtaagatgcttttctgtgactggtgagtactcaaccaagtcattctga
gaatagtgtatgcggcgaccgagttgctcttgcccggcgtcaatacgggataataccgcgccacatag
cagaactttaaaagtgctcatcattggaaaacgttcttcggggcgaaaactctcaaggatcttaccgc
tgttgagatccagttcgatgtaacccactcgtgcacccaactgatcttcagcatcttttactttcacc
agcgtttctgggtgagcaaaaacaggaaggcaaaatgccgcaaaaaagggaataagggcgacacggaa
atgttgaatactcatactcttcctttttcaatattattgaagcatttatcagggttattgtctcatga
gcggatacatatttgaatgtatttagaaaaataaacaaataggggttccgcgcacatttccccgaaaa
gtgccacctgacgtc
PVP36b SARS-COV-2 UTR Spike delta Hexapro
(SEQ ID NO: 42)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATgTTTGTGTTCCTGGTGCTGCTGCCACTGGTGTCCAGCCAGTGTGTGAACCTGAGGACCAG
GACCCAACTTCCTCCTGCCTACACCAACTCCTTCACCAGGGGAGTCTACTACCCTGACAAGGTGTTCA
GGTCCTCTGTGCTGCACAGCACCCAGGACCTGTTCCTGCCATTCTTCAGCAATGTGACCTGGTTCCAT
GCCATCCATGTGTCTGGCACCAATGGCACCAAGAGGTTTGACAACCCTGTGCTGCCATTCAATGATGG
AGTCTACTTTGCCAGCACAGAGAAGAGCAACATCATCAGGGGCTGGATTTTTGGCACCACCCTGGACA
GCAAGACCCAGTCCCTGCTGATTGTGAACAATGCCACCAATGTGGTGATTAAGGTGTGTGAGTTCCAG
TTCTGTAATGACCCATTCCTGGACGTCTACTACCACAAGAACAACAAGTCCTGGATGGAGTCTGGCGT
CTACTCCTCTGCCAACAACTGTACCTTTGAATATGTGAGCCAACCATTCCTGATGGACTTGGAGGGCA
AGCAGGGCAACTTCAAGAACCTGAGGGAGTTTGTGTTCAAGAACATTGATGGCTACTTCAAGATTTAC
AGCAAACACACACCAATCAACCTGGTGAGGGACCTGCCACAGGGCTTCTCTGCCTTGGAACCACTGGT
GGACCTGCCAATTGGCATCAACATCACCAGGTTCCAGACCCTGCTGGCTCTGCACAGGTCCTACCTGA
CACCTGGAGACTCCTCCTCTGGCTGGACAGCAGGAGCAGCAGCCTACTATGTGGGCTACCTCCAACCA
AGGACCTTCCTGCTGAAATACAATGAGAATGGCACCATCACAGATGCTGTGGACTGTGCCCTGGACCC
ACTGTCTGAGACCAAGTGTACCCTGAAATCCTTCACAGTGGAGAAGGGCATCTACCAGACCAGCAACT
TCAGGGTCCAACCAACAGAGAGCATTGTGAGGTTTCCAAACATCACCAACCTGTGTCCATTTGGAGAG
GTGTTCAATGCCACCAGGTTTGCCTCTGTCTATGCCTGGAACAGGAAGAGGATTAGCAACTGTGTGGC
TGACTACTCTGTGCTCTACAACTCTGCCTCCTTCAGCACCTTCAAGTGTTATGGAGTGAGCCCAACCA
AACTGAATGACCTGTGTTTCACCAATGTCTATGCTGACTCCTTTGTGATTAGGGGAGATGAGGTGAGA
CAGATTGCCCCTGGACAAACAGGCAAGATTGCTGACTACAACTACAAACTGCCTGATGACTTCACAGG
CTGTGTGATTGCCTGGAACAGCAACAACCTGGACAGCAAGGTGGGAGGCAACTACAACTACAGGTACA
GACTGTTCAGGAAGAGCAACCTGAAACCATTTGAGAGGGACATCAGCACAGAGATTTACCAGGCTGGC
AGCAAGCCATGTAATGGAGTGGAGGGCTTCAACTGTTACTTTCCACTCCAATCCTATGGCTTCCAACC
AACCAATGGAGTGGGCTACCAACCATACAGGGTGGTGGTGCTGTCCTTTGAACTGCTCCATGCCCCTG
CCACAGTGTGTGGACCAAAGAAGAGCACCAACCTGGTGAAGAACAAGTGTGTGAACTTCAACTTCAAT
GGACTGACAGGCACAGGAGTGCTGACAGAGAGCAACAAGAAGTTCCTGCCATTCCAACAGTTTGGCAG
GGACATTGCTGACACCACAGATGCTGTGAGGGACCCACAGACCTTGGAGATTCTGGACATCACACCAT
GTTCCTTTGGAGGAGTGTCTGTGATTACACCTGGCACCAACACCAGCAACCAGGTGGCTGTGCTCTAC
CAGGGCGTGAACTGTACTGAGGTGCCTGTGGCTATCCATGCTGACCAACTTACACCAACCTGGAGGGT
CTACAGCACAGGAAGCAACGTGTTCCAGACCAGGGCTGGCTGTCTGATTGGAGCAGAGCATGTGAACA
ACTCCTATGAGTGTGACATCCCAATTGGAGCAGGCATCTGTGCCTCCTACCAGACCCAGACCAACAGC
AGGGGCTCTGCATCTTCTGTGGCAAGCCAGAGCATCATTGCCTACACAATGAGTCTGGGAGCAGAGAA
CTCTGTGGCTTACAGCAACAACAGCATTGCCATCCCAACCAACTTCACCATCTCTGTGACCACAGAGA
TTCTGCCTGTGAGTATGACCAAGACCTCTGTGGACTGTACAATGTATATCTGTGGAGACAGCACAGAG
TGTAGCAACCTGCTGCTCCAATATGGCTCCTTCTGTACCCAACTTAACAGGGCTCTGACAGGCATTGC
TGTGGAACAGGACAAGAACACCCAGGAGGTGTTTGCCCAGGTGAAGCAGATTTACAAGACACCTCCAA
TCAAGGACTTTGGAGGCTTCAACTTCAGCCAGATTCTGCCTGACCCAAGCAAGCCAAGCAAGAGGTCC
CCTATTGAGGACCTGCTGTTCAACAAGGTGACCCTGGCTGATGCTGGCTTCATCAAGCAATATGGAGA
CTGTCTGGGAGACATTGCCGCCAGGGACCTGATTTGTGCCCAGAAGTTCAATGGACTGACAGTGCTGC
CTCCACTGCTGACAGATGAGATGATTGCCCAATACACCTCTGCCCTGCTGGCTGGCACCATCACCTCT
GGCTGGACCTTTGGAGCAGGACCAGCCCTCCAAATCCCATTTCCAATGCAGATGGCTTACAGGTTCAA
TGGCATTGGAGTGACCCAGAATGTGCTCTATGAGAACCAGAAACTGATTGCCAACCAGTTCAACTCTG
CCATTGGCAAGATTCAGGACTCCCTGTCCAGCACACCATCTGCCCTGGGCAAACTCCAAAACGTGGTG
AACCAGAATGCCCAGGCTCTGAACACCCTGGTGAAGCAACTTTCCAGCAACTTTGGAGCCATCTCCTC
TGTGCTGAATGACATCCTGAGCAGACTGGACCCACCAGAGGCTGAGGTCCAGATTGACAGACTGATTA
CAGGCAGACTCCAATCCCTCCAAACCTATGTGACCCAACAACTTATCAGGGCTGCTGAGATTAGGGCA
TCTGCCAACCTGGCTGCCACCAAGATGAGTGAGTGTGTGCTGGGACAAAGCAAGAGGGTGGACTTCTG
TGGCAAGGGCTACCACCTGATGAGTTTTCCACAGTCTGCCCCTCATGGAGTGGTGTTCCTGCATGTGA
CCTATGTGCCTGCCCAGGAGAAGAACTTCACCACAGCCCCTGCCATCTGCCATGATGGCAAGGCTCAC
TTTCCAAGGGAGGGAGTGTTTGTGAGCAATGGCACCCACTGGTTTGTGACCCAGAGGAACTTCTATGA
ACCACAGATTATCACCACAGACAACACCTTTGTGTCTGGCAACTGTGATGTGGTGATTGGCATTGTGA
ACAACACAGTCTATGACCCACTCCAACCTGAACTGGACTCCTTCAAGGAGGAACTGGACAAATACTTC
AAGAACCACACCAGCCCTGATGTGGACCTGGGAGACATCTCTGGCATCAATGCCTCTGTGGTGAACAT
CCAGAAGGAGATTGACAGACTGAATGAGGTGGCTAAGAACCTGAATGAGTCCCTGATTGACCTCCAAG
AACTGGGCAAATATGAACAATACATCAAGTGGCCATGGTACATCTGGCTGGGCTTCATTGCTGGACTG
ATTGCCATTGTGATGGTGACCATAATGCTGTGTTGTATGACCTCCTGTTGTTCCTGTCTGAAAGGCTG
TTGTTCCTGTGGCTCCTGTTGTAAGTTTGATGAGGATGACTCTGAACCTGTGCTGAAAGGAGTGAAAC
TGCACTACACCTGACTCGAGCTGGTACTGCATGCACGCAATGCTAGCTGCCCCTTTCCCGTCCTGGGT
ACCCCGAGTCTCCCCCGACCTCGGGTCCCAGGTATGCTCCCACCTCCACCTGCCCCACTCACCACCTC
TGCTAGTTCCAGACACCTCCCAAGCACGCAGCAATGCAGCTCAAAACGCTTAGCCTAGCCACACCCCC
ACGGGAAACAGCAGTGATTAACCTTTAGCAATAAACGAAAGTTTAACTAAGCTATACTAACCCCAGGG
TTGGTCAATTTCGTGCCAGCCACACCCTGGAGCTAGCaaaccgctgatcagcctcgactgtgccttct
agttgccagccatctgttgtttgcccctcccccgtgccttccttgaccctggaaggtgccactcccac
tgtcctttcctaataaaatgaggaaattgcatcgcattgtctgagtaggtgtcattctattctggggg
gtggggtggggcaggacagcaagggggaggattgggaagacaatagcaggcatgctggggatgcggtg
ggctctatggcttctgaggcggaaagaaccagctggggctctagggggtatccccacgcgccctgtag
cggcgcattaagcgcggcgggtgtggtggttacgcgcagcgtgaccgctacacttgccagcgccctag
cgcccgctcctttcgctttcttcccttcctttctcgccacgttcgccggctttccccgtcaagctcta
aatcgggggctccctttagggttccgatttagtgctttacggcacctcgaccccaaaaaacttgatta
gggtgatggttcacgtagtgggccatcgccctgatagacggtttttcgccctttgacgttggagtcca
cgttctttaatagtggactcttgttccaaactggaacaacactcaaccctatctcggtctattctttt
gatttataagggattttgccgatttcggcctattggttaaaaaatgagctgatttaacaaaaatttaa
cgcgaattaattctgtggaatgtgtgtcagttagggtgtggaaagtccccaggctccccagcaggcag
aagtatgcaaagcatgcatctcaattagtcagcaaccaggtgtggaaagtccccaggctccccagcag
gcagaagtatgcaaagcatgcatctcaattagtcagcaaccatagtcccgcccctaactccgcccatc
ccgcccctaactccgcccagttccgcccattctccgccccatggctgactaattttttttatttatgc
agaggccgaggccgcctctgcctctgagctattccagaagtagtgaggaggcttttttggaggcctag
gcttttgcaaaaagctcccgggagcttgtatatccattttcggatctgatcaagagacaggatgagga
tcgtttcgcatgattgaacaagatggattgcacgcaggttctccggccgcttgggtggagaggctatt
cggctatgactgggcacaacagacaatcggctgctctgatgccgccgtgttccggctgtcagcgcagg
ggcgcccggttctttttgtcaagaccgacctgtccggtgccctgaatgaactgcaggacgaggcagcg
cggctatcgtggctggccacgacgggcgttccttgcgcagctgtgctcgacgttgtcactgaagcggg
aagggactggctgctattgggcgaagtgccggggcaggatctcctgtcatctcaccttgctcctgccg
agaaagtatccatcatggctgatgcaatgcggcggctgcatacgcttgatccggctacctgcccattc
gaccaccaagcgaaacatcgcatcgagcgagcacgtactcggatggaagccggtcttgtcgatcagga
tgatctggacgaagagcatcaggggctcgcgccagccgaactgttcgccaggctcaaggcgcgcatgc
ccgacggcgaggatctcgtcgtgacccatggcgatgcctgcttgccgaatatcatggtggaaaatggc
cgcttttctggattcatcgactgtggccggctgggtgtggcggaccgctatcaggacatagcgttggc
tacccgtgatattgctgaagagcttggcggcgaatgggctgaccgcttcctcgtgctttacggtatcg
ccgctcccgattcgcagcgcatcgccttctatcgccttcttgacgagttcttctgagcgggactctgg
ggttcgaaatgaccgaccaagcgacgcccaacctgccatcacgagatttcgattccaccgccgccttc
tatgaaaggttgggcttcggaatcgttttccgggacgccggctggatgatcctccagcgcggggatct
catgctggagttcttcgcccaccccaacttgtttattgcagcttataatggttacaaataaagcaata
gcatcacaaatttcacaaataaagcatttttttcactgcattctagttgtggtttgtccaaactcatc
aatgtatcttatcatgtctgtataccgtcgacctctagctagagcttggcgtaatcatggtcatagct
gtttcctgtgtgaaattgttatccgctcacaattccacacaacatacgagccggaagcataaagtgta
aagcctggggtgcctaatgagtgagctaactcacattaattgcgttgcgctcactgcccgctttccag
tcgggaaacctgtcgtgccagctgcattaatgaatcggccaacgcgcggggagaggcggtttgcgtat
tgggcgctcttccgcttcctcgctcactgactcgctgcgctcggtcgttcggctgcggcgagcggtat
cagctcactcaaaggcggtaatacggttatccacagaatcaggggataacgcaggaaagaacatgtga
gcaaaaggccagcaaaaggccaggaaccgtaaaaaggccgcgttgctggcgtttttccataggctccg
cccccctgacgagcatcacaaaaatcgacgctcaagtcagaggtggcgaaacccgacaggactataaa
gataccaggcgtttccccctggaagctccctcgtgcgctctcctgttccgaccctgccgcttaccgga
tacctgtccgcctttctcccttcgggaagcgtggcgctttctcatagctcacgctgtaggtatctcag
ttcggtgtaggtcgttcgctccaagctgggctgtgtgcacgaaccccccgttcagcccgaccgctgcg
ccttatccggtaactatcgtcttgagtccaacccggtaagacacgacttatcgccactggcagcagcc
actggtaacaggattagcagagcgaggtatgtaggcggtgctacagagttcttgaagtggtggcctaa
ctacggctacactagaagaacagtatttggtatctgcgctctgctgaagccagttaccttcggaaaaa
gagttggtagctcttgatccggcaaacaaaccaccgctggtagcggtggtttttttgtttgcaagcag
cagattacgcgcagaaaaaaaggatctcaagaagatcctttgatcttttctacggggtctgacgctca
gtggaacgaaaactcacgttaagggattttggtcatgagattatcaaaaaggatcttcacctagatcc
ttttaaattaaaaatgaagttttaaatcaatctaaagtatatatgagtaaacttggtctgacagttac
caatgcttaatcagtgaggcacctatctcagcgatctgtctatttcgttcatccatagttgcctgact
ccccgtcgtgtagataactacgatacgggagggcttaccatctggccccagtgctgcaatgataccgc
gagacccacgctcaccggctccagatttatcagcaataaaccagccagccggaagggccgagcgcaga
agtggtcctgcaactttatccgcctccatccagtctattaattgttgccgggaagctagagtaagtag
ttcgccagttaatagtttgcgcaacgttgttgccattgctacaggcatcgtggtgtcacgctcgtcgt
ttggtatggcttcattcagctccggttcccaacgatcaaggcgagttacatgatcccccatgttgtgc
aaaaaagcggttagctccttcggtcctccgatcgttgtcagaagtaagttggccgcagtgttatcact
catggttatggcagcactgcataattctcttactgtcatgccatccgtaagatgcttttctgtgactg
gtgagtactcaaccaagtcattctgagaatagtgtatgcggcgaccgagttgctcttgcccggcgtca
atacgggataataccgcgccacatagcagaactttaaaagtgctcatcattggaaaacgttcttcggg
gcgaaaactctcaaggatcttaccgctgttgagatccagttcgatgtaacccactcgtgcacccaact
gatcttcagcatcttttactttcaccagcgtttctgggtgagcaaaaacaggaaggcaaaatgccgca
aaaaagggaataagggcgacacggaaatgttgaatactcatactcttcctttttcaatattattgaag
catttatcagggttattgtctcatgagcggatacatatttgaatgtatttagaaaaataaacaaatag
gggttccgcgcacatttccccgaaaagtgccacctgacgtc
PVP37 SARSCOV Pseudovirus spike deltaC 19
(SEQ ID NO: 43)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccATgTTCATCTTCCTGCTGTTCCTGACCCTGACCTCTGGCTCTGACCTG
GACAGGTGTACCACCTTTGATGATGTCCAGGCTCCAAACTACACCCAACACACCTCCAGTATGAGGGG
AGTCTACTACCCTGATGAGATTTTCAGGTCTGACACCCTCTACCTGACCCAGGACCTGTTCCTGCCAT
TCTACAGCAATGTGACAGGCTTCCACACCATCAACCACACCTTTGACAACCCTGTGATTCCATTCAAG
GATGGCATCTACTTTGCTGCCACAGAGAAGAGCAATGTGGTGAGGGGCTGGGTGTTTGGCAGCACAAT
GAACAACAAGAGCCAGTCTGTGATTATCATCAACAACAGCACCAATGTGGTGATTAGGGCTTGTAACT
TTGAACTGTGTGACAACCCATTCTTTGCTGTGAGCAAGCCTATGGGCACCCAGACCCACACAATGATT
TTTGACAATGCCTTCAACTGTACCTTTGAATACATCTCTGATGCCTTCTCCCTGGATGTGTCTGAGAA
GTCTGGCAACTTCAAACACCTGAGGGAGTTTGTGTTCAAGAACAAGGATGGCTTCCTCTATGTCTACA
AGGGCTACCAACCAATTGATGTGGTGAGGGACCTGCCATCTGGCTTCAACACCCTGAAACCAATCTTC
AAACTGCCACTGGGCATCAACATCACCAACTTCAGGGCTATCCTGACAGCCTTCAGCCCTGCCCAGGA
CACCTGGGGCACCTCTGCTGCTGCCTACTTTGTGGGCTACCTGAAACCAACCACCTTTATGCTGAAAT
ATGATGAGAATGGCACCATCACAGATGCTGTGGACTGTAGCCAGAACCCACTGGCTGAACTGAAATGT
TCTGTGAAGTCCTTTGAGATTGACAAGGGCATCTACCAGACCAGCAACTTCAGGGTGGTGCCATCTGG
AGATGTGGTGAGGTTTCCAAACATCACCAACCTGTGTCCATTTGGAGAGGTGTTCAATGCCACCAAGT
TTCCATCTGTCTATGCCTGGGAGAGGAAGAAGATTAGCAACTGTGTGGCTGACTACTCTGTGCTCTAC
AACAGCACCTTCTTCAGCACCTTCAAGTGTTATGGAGTGTCTGCCACCAAACTGAATGACCTGTGTTT
CAGCAATGTCTATGCTGACTCCTTTGTGGTGAAGGGAGATGATGTGAGACAGATTGCCCCTGGACAAA
CAGGAGTGATTGCTGACTACAACTACAAACTGCCTGATGACTTTATGGGCTGTGTGCTGGCTTGGAAC
ACCAGGAACATTGATGCCACCAGCACAGGCAACTACAACTACAAATACAGATACCTGAGACATGGCAA
ACTGAGACCATTTGAGAGGGACATCAGCAATGTGCCATTCAGCCCTGATGGCAAGCCATGTACTCCTC
CTGCCCTGAACTGTTACTGGCCACTGAATGACTATGGCTTCTACACCACCACAGGCATTGGCTACCAA
CCATACAGGGTGGTGGTGCTGTCCTTTGAACTGCTGAATGCCCCTGCCACAGTGTGTGGACCAAAACT
GAGCACAGACCTGATTAAGAACCAGTGTGTGAACTTCAACTTCAATGGACTGACAGGCACAGGAGTGC
TGACACCATCCAGCAAGAGGTTCCAACCATTCCAACAGTTTGGCAGGGATGTGTCTGACTTCACAGAC
TCTGTGAGGGACCCAAAGACCTCTGAGATTCTGGACATCAGCCCATGTTCCTTTGGAGGAGTGTCTGT
GATTACACCTGGCACCAATGCCTCCTCTGAGGTGGCTGTGCTCTACCAGGATGTGAACTGTACTGATG
TGAGCACAGCCATCCATGCTGACCAACTTACACCTGCCTGGAGGATTTACAGCACAGGCAACAATGTG
TTCCAGACCCAGGCTGGCTGTCTGATTGGAGCAGAGCATGTGGACACCTCCTATGAGTGTGACATCCC
AATTGGAGCAGGCATCTGTGCCTCCTACCACACAGTGTCCCTGCTGAGGAGCACCAGCCAGAAGAGCA
TTGTGGCTTACACAATGAGTCTGGGAGCAGACTCCAGCATTGCCTACAGCAACAACACCATTGCCATC
CCAACCAACTTCAGCATCAGCATCACCACAGAGGTGATGCCTGTGAGTATGGCTAAGACCTCTGTGGA
CTGTAATATGTATATCTGTGGAGACAGCACAGAGTGTGCCAACCTGCTGCTCCAATATGGCTCCTTCT
GTACCCAACTTAACAGGGCTCTGTCTGGCATTGCTGCTGAACAGGACAGGAACACCAGGGAGGTGTTT
GCCCAGGTGAAGCAGATGTATAAGACACCAACCCTGAAATACTTTGGAGGCTTCAACTTCAGCCAGAT
TCTGCCTGACCCACTGAAACCAACCAAGAGGTCCTTCATTGAGGACCTGCTGTTCAACAAGGTGACCC
TGGCTGATGCTGGCTTTATGAAGCAATATGGAGAGTGTCTGGGAGACATCAATGCCAGGGACCTGATT
TGTGCCCAGAAGTTCAATGGACTGACAGTGCTGCCTCCACTGCTGACAGATGATATGATTGCTGCCTA
CACAGCAGCCCTGGTGTCTGGCACAGCCACAGCAGGCTGGACCTTTGGAGCAGGAGCAGCCCTCCAAA
TCCCATTTGCTATGCAGATGGCTTACAGGTTCAATGGCATTGGAGTGACCCAGAATGTGCTCTATGAG
AACCAGAAGCAGATTGCCAACCAGTTCAACAAGGCTATCAGCCAGATTCAGGAGTCCCTGACCACCAC
CAGCACAGCCCTGGGCAAACTCCAAGATGTGGTGAACCAGAATGCCCAGGCTCTGAACACCCTGGTGA
AGCAACTTTCCAGCAACTTTGGAGCCATCTCCTCTGTGCTGAATGACATCCTGAGCAGACTGGACAAG
GTGGAGGCTGAGGTCCAGATTGACAGACTGATTACAGGCAGACTCCAATCCCTCCAAACCTATGTGAC
CCAACAACTTATCAGGGCTGCTGAGATTAGGGCATCTGCCAACCTGGCTGCCACCAAGATGAGTGAGT
GTGTGCTGGGACAAAGCAAGAGGGTGGACTTCTGTGGCAAGGGCTACCACCTGATGAGTTTTCCACAG
GCTGCCCCTCATGGAGTGGTGTTCCTGCATGTGACCTATGTGCCAAGCCAGGAGAGGAACTTCACCAC
AGCCCCTGCCATCTGCCATGAGGGCAAGGCTTACTTTCCAAGGGAGGGAGTGTTTGTGTTCAATGGCA
CCTCCTGGTTCATCACCCAGAGGAACTTCTTCAGCCCACAGATTATCACCACAGACAACACCTTTGTG
TCTGGCAACTGTGATGTGGTGATTGGCATCATCAACAACACAGTCTATGACCCACTCCAACCTGAACT
GGACTCCTTCAAGGAGGAACTGGACAAATACTTCAAGAACCACACCAGCCCTGATGTGGACCTGGGAG
ACATCTCTGGCATCAATGCCTCTGTGGTGAACATCCAGAAGGAGATTGACAGACTGAATGAGGTGGCT
AAGAACCTGAATGAGTCCCTGATTGACCTCCAAGAACTGGGCAAATATGAACAATACATCAAGTGGCC
ATGGTATGTGTGGCTGGGCTTCATTGCTGGACTGATTGCCATTGTGATGGTGACCATCCTGCTGTGTT
GTATGACCTCCTGTTGTTCCTGTCTGAAAGGAGCCTGTTCCTGTGGCTCCTGTTGTTAAggatccgag
ctcggtaccaagcttaagtttaaaccgctgatcagcctcgactgtgccttctagttgccagccatctg
ttgtttgcccctcccccgtgccttccttgaccctggaaggtgccactcccactgtcctttcctaataa
aatgaggaaattgcatcgcattgtctgagtaggtgtcattctattctggggggtggggtggggcagga
cagcaagggggaggattgggaagacaatagcaggcatgctggggatgcggtgggctctatggcttctg
aggcggaaagaaccagctggggctctagggggtatccccacgcgccctgtagcggcgcattaagcgcg
gcgggtgtggtggttacgcgcagcgtgaccgctacacttgccagcgccctagcgcccgctcctttcgc
tttcttcccttcctttctcgccacgttcgccggctttccccgtcaagctctaaatcgggggctccctt
tagggttccgatttagtgctttacggcacctcgaccccaaaaaacttgattagggtgatggttcacgt
agtgggccatcgccctgatagacggtttttcgccctttgacgttggagtccacgttctttaatagtgg
actcttgttccaaactggaacaacactcaaccctatctcggtctattcttttgatttataagggattt
tgccgatttcggcctattggttaaaaaatgagctgatttaacaaaaatttaacgcgaattaattctgt
ggaatgtgtgtcagttagggtgtggaaagtccccaggctccccagcaggcagaagtatgcaaagcatg
catctcaattagtcagcaaccaggtgtggaaagtccccaggctccccagcaggcagaagtatgcaaag
catgcatctcaattagtcagcaaccatagtcccgcccctaactccgcccatcccgcccctaactccgc
ccagttccgcccattctccgccccatggctgactaattttttttatttatgcagaggccgaggccgcc
tctgcctctgagctattccagaagtagtgaggaggcttttttggaggcctaggcttttgcaaaaagct
cccgggagcttgtatatccattttcggatctgatcaagagacaggatgaggatcgtttcgcatgattg
aacaagatggattgcacgcaggttctccggccgcttgggtggagaggctattcggctatgactgggca
caacagacaatcggctgctctgatgccgccgtgttccggctgtcagcgcaggggcgcccggttctttt
tgtcaagaccgacctgtccggtgccctgaatgaactgcaggacgaggcagcgcggctatcgtggctgg
ccacgacgggcgttccttgcgcagctgtgctcgacgttgtcactgaagcgggaagggactggctgcta
ttgggcgaagtgccggggcaggatctcctgtcatctcaccttgctcctgccgagaaagtatccatcat
ggctgatgcaatgcggcggctgcatacgcttgatccggctacctgcccattcgaccaccaagcgaaac
atcgcatcgagcgagcacgtactcggatggaagccggtcttgtcgatcaggatgatctggacgaagag
catcaggggctcgcgccagccgaactgttcgccaggctcaaggcgcgcatgcccgacggcgaggatct
cgtcgtgacccatggcgatgcctgcttgccgaatatcatggtggaaaatggccgcttttctggattca
tcgactgtggccggctgggtgtggcggaccgctatcaggacatagcgttggctacccgtgatattgct
gaagagcttggcggcgaatgggctgaccgcttcctcgtgctttacggtatcgccgctcccgattcgca
gcgcatcgccttctatcgccttcttgacgagttcttctgagcgggactctggggttcgaaatgaccga
ccaagcgacgcccaacctgccatcacgagatttcgattccaccgccgccttctatgaaaggttgggct
tcggaatcgttttccgggacgccggctggatgatcctccagcgcggggatctcatgctggagttcttc
gcccaccccaacttgtttattgcagcttataatggttacaaataaagcaatagcatcacaaatttcac
aaataaagcatttttttcactgcattctagttgtggtttgtccaaactcatcaatgtatcttatcatg
tctgtataccgtcgacctctagctagagcttggcgtaatcatggtcatagctgtttcctgtgtgaaat
tgttatccgctcacaattccacacaacatacgagccggaagcataaagtgtaaagcctggggtgccta
atgagtgagctaactcacattaattgcgttgcgctcactgcccgctttccagtcgggaaacctgtcgt
gccagctgcattaatgaatcggccaacgcgcggggagaggcggtttgcgtattgggcgctcttccgct
tcctcgctcactgactcgctgcgctcggtcgttcggctgcggcgagcggtatcagctcactcaaaggc
ggtaatacggttatccacagaatcaggggataacgcaggaaagaacatgtgagcaaaaggccagcaaa
aggccaggaaccgtaaaaaggccgcgttgctggcgtttttccataggctccgcccccctgacgagcat
cacaaaaatcgacgctcaagtcagaggtggcgaaacccgacaggactataaagataccaggcgtttcc
ccctggaagctccctcgtgcgctctcctgttccgaccctgccgcttaccggatacctgtccgcctttc
tcccttcgggaagcgtggcgctttctcatagctcacgctgtaggtatctcagttcggtgtaggtcgtt
cgctccaagctgggctgtgtgcacgaaccccccgttcagcccgaccgctgcgccttatccggtaacta
tcgtcttgagtccaacccggtaagacacgacttatcgccactggcagcagccactggtaacaggatta
gcagagcgaggtatgtaggcggtgctacagagttcttgaagtggtggcctaactacggctacactaga
agaacagtatttggtatctgcgctctgctgaagccagttaccttcggaaaaagagttggtagctcttg
atccggcaaacaaaccaccgctggtagcggtggtttttttgtttgcaagcagcagattacgcgcagaa
aaaaaggatctcaagaagatcctttgatcttttctacggggtctgacgctcagtggaacgaaaactca
cgttaagggattttggtcatgagattatcaaaaaggatcttcacctagatccttttaaattaaaaatg
aagttttaaatcaatctaaagtatatatgagtaaacttggtctgacagttaccaatgcttaatcagtg
aggcacctatctcagcgatctgtctatttcgttcatccatagttgcctgactccccgtcgtgtagata
actacgatacgggagggcttaccatctggccccagtgctgcaatgataccgcgagacccacgctcacc
ggctccagatttatcagcaataaaccagccagccggaagggccgagcgcagaagtggtcctgcaactt
tatccgcctccatccagtctattaattgttgccgggaagctagagtaagtagttcgccagttaatagt
ttgcgcaacgttgttgccattgctacaggcatcgtggtgtcacgctcgtcgtttggtatggcttcatt
cagctccggttcccaacgatcaaggcgagttacatgatcccccatgttgtgcaaaaaagcggttagct
ccttcggtcctccgatcgttgtcagaagtaagttggccgcagtgttatcactcatggttatggcagca
ctgcataattctcttactgtcatgccatccgtaagatgcttttctgtgactggtgagtactcaaccaa
gtcattctgagaatagtgtatgcggcgaccgagttgctcttgcccggcgtcaatacgggataataccg
cgccacatagcagaactttaaaagtgctcatcattggaaaacgttcttcggggcgaaaactctcaagg
atcttaccgctgttgagatccagttcgatgtaacccactcgtgcacccaactgatcttcagcatcttt
tactttcaccagcgtttctgggtgagcaaaaacaggaaggcaaaatgccgcaaaaaagggaataaggg
cgacacggaaatgttgaatactcatactcttcctttttcaatattattgaagcatttatcagggttat
tgtctcatgagcggatacatatttgaatgtatttagaaaaataaacaaataggggttccgcgcacatt
tccccgaaaagtgccacctgacgtc
pVP38 MERSCoV Pseudovirus spike deltaC-16
(SEQ ID NO: 44)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccATgATTCACTCTGTGTTCCTGCTGATGTTCCTGCTGACACCAACAGAG
TCCTATGTGGATGTGGGACCTGACTCTGTGAAGTCTGCCTGTATTGAGGTGGACATCCAACAGACCTT
CTTTGACAAGACCTGGCCAAGACCAATTGATGTGAGCAAGGCTGATGGCATCATCTACCCACAGGGCA
GGACCTACAGCAACATCACCATCACCTACCAGGGACTGTTTCCATACCAGGGAGATCATGGAGATATG
TATGTCTACTCTGCTGGTCATGCCACAGGCACCACACCACAGAAACTGTTTGTGGCTAACTACAGCCA
GGATGTGAAGCAGTTTGCCAATGGCTTTGTGGTGAGGATTGGAGCAGCAGCCAACAGCACAGGCACAG
TGATTATCAGCCCAAGCACCTCTGCCACCATCAGGAAGATTTACCCTGCCTTTATGCTGGGCTCCTCT
GTGGGCAACTTCTCTGATGGCAAGATGGGCAGGTTCTTCAACCACACCCTGGTGCTGCTGCCTGATGG
CTGTGGCACCCTGCTGAGGGCTTTCTACTGTATCTTGGAACCAAGGTCTGGCAACCACTGTCCTGCTG
GCAACTCCTACACCTCCTTTGCCACCTACCACACACCTGCCACAGACTGTTCTGATGGCAACTACAAC
AGGAATGCCTCCCTGAACTCCTTCAAGGAATACTTCAACCTGAGGAACTGTACCTTTATGTACACCTA
CAACATCACAGAGGATGAGATTTTGGAGTGGTTTGGCATCACCCAGACAGCCCAGGGAGTGCATCTGT
TCTCGAGCAGATATGTGGACCTCTATGGAGGCAATATGTTCCAGTTTGCCACCCTGCCTGTCTATGAC
ACCATCAAATACTACAGCATCATCCCACACAGCATCAGGAGCATCCAGTCTGACAGGAAGGCTTGGGC
TGCCTTCTATGTCTACAAACTCCAACCACTGACCTTCCTGCTGGACTTCTCTGTGGATGGCTACATCA
GGAGGGCTATTGACTGTGGCTTCAATGACCTGAGCCAACTTCACTGTTCCTATGAGTCCTTTGATGTG
GAGTCTGGAGTCTACTCTGTGTCCTCCTTTGAGGCTAAGCCATCTGGCTCTGTGGTGGAACAGGCTGA
GGGAGTGGAGTGTGACTTCAGCCCACTGCTGTCTGGCACACCTCCACAGGTCTACAACTTCAAGAGAC
TGGTGTTCACCAACTGTAACTACAACCTGACCAAACTGCTGTCCCTGTTCTCTGTGAATGACTTCACT
TGTAGCCAGATTAGCCCTGCTGCCATTGCCAGCAACTGTTACTCCTCCCTGATTCTGGACTACTTCTC
CTACCCACTGAGTATGAAGTCTGACCTGTCTGTGTCCTCTGCTGGACCAATCAGCCAGTTCAACTACA
AGCAGTCCTTCAGCAACCCAACTTGTCTGATTCTGGCTACAGTGCCACACAACCTGACCACCATCACC
AAGCCACTGAAATACTCCTACATCAACAAGTGTAGCAGACTGCTGTCTGATGACAGGACAGAGGTGCC
ACAACTAGTGAATGCCAACCAATACAGCCCATGTGTGAGCATTGTGCCAAGCACAGTGTGGGAGGATG
GAGACTACTACAGGAAGCAACTTAGCCCATTGGAGGGAGGAGGCTGGCTGGTGGCATCTGGCAGCACA
GTGGCTATGACAGAACAACTCCAAATGGGCTTTGGCATCACAGTCCAATATGGCACAGACACCAACTC
TGTGTGTCCAAAATTGGAGTTTGCCAATGACACCAAGATTGCCAGCCAACTTGGCAACTGTGTGGAAT
ACTCCCTCTATGGAGTGTCTGGCAGGGGAGTGTTCCAGAACTGTACTGCTGTGGGAGTGAGACAACAG
AGGTTTGTCTATGATGCCTACCAGAACCTGGTGGGCTACTACTCTGATGATGGCAACTACTACTGTCT
GAGGGCTTGTGTGTCTGTGCCTGTGTCTGTGATTTATGACAAGGAGACCAAGACCCATGCCACCCTGT
TTGGCTCTGTGGCTTGTGAACACATCTCCAGCACAATGAGTCAATACAGCAGGAGCACCAGGAGTATG
CTGAAAAGGAGGGACAGCACATATGGACCACTCCAAACACCTGTGGGCTGTGTGCTGGGACTGGTGAA
CTCCTCCCTGTTTGTGGAGGACTGTAAACTGCCACTGGGACAATCCCTGTGTGCCCTGCCTGACACAC
CAAGCACCCTGACACCAAGGTCTGTGAGGTCTGTGCCTGGAGAGATGAGACTGGCAAGCATTGCCTTC
AACCACCCAATCCAGGTGGACCAACTTAACTCCTCCTACTTCAAACTGAGCATCCCAACCAACTTCTC
CTTTGGAGTGACCCAGGAATACATCCAGACCACCATCCAGAAGGTGACAGTGGACTGTAAGCAATATG
TGTGTAATGGCTTCCAGAAGTGTGAACAACTTCTGAGGGAATATGGACAATTCTGTAGCAAGATAAAC
CAGGCTCTTCATGGAGCCAACCTGAGACAGGATGACTCTGTGAGGAACCTGTTTGCCTCTGTGAAGTC
CAGCCAGTCCAGCCCAATCATCCCTGGCTTTGGAGGAGACTTCAACCTGACCCTGTTGGAACCGGTGA
GCATCAGCACAGGCAGCAGGTCTGCCAGGTCTGCCATTGAGGACCTGCTGTTTGACAAGGTGACCATT
GCTGACCCTGGCTATATGCAGGGCTATGATGACTGTATGCAACAGGGACCTGCCTCTGCCAGGGACCT
GATTTGTGCCCAATATGTGGCTGGCTACAAGGTGCTGCCTCCACTGATGGATGTGAATATGGAGGCTG
CCTACACCTCCTCCCTGCTGGGCAGCATTGCTGGAGTGGGCTGGACTGCAGGACTGTCCTCCTTTGCT
GCCATCCCATTTGCCCAGAGCATCTTCTACAGACTGAATGGAGTGGGCATCACCCAACAGGTGCTGTC
TGAGAACCAGAAACTGATTGCCAACAAGTTCAACCAGGCTCTGGGAGCTATGCAGACAGGCTTCACCA
CCACCAATGAGGCTTTCCAGAAGGTCCAGGATGCTGTGAACAACAATGCCCAGGCTCTGAGCAAACTG
GCATCTGAACTGAGCAACACCTTTGGAGCCATCTCTGCTAGCATTGGAGACATCATCCAGAGACTGGA
TGTGTTGGAACAGGATGCCCAGATTGACAGACTGATAAATGGCAGACTGACCACCCTGAATGCCTTTG
TGGCTCAACAACTTGTGAGGTCTGAGTCTGCTGCCCTGTCTGCCCAACTTGCCAAGGACAAGGTGAAT
GAGTGTGTGAAGGCTCAAAGCAAGAGGTCTGGCTTCTGTGGACAAGGCACCCACATTGTGTCCTTTGT
GGTGAATGCCCCAAATGGACTCTACTTTATGCATGTGGGCTACTACCCAAGCAACCACATTGAGGTGG
TGTCTGCCTATGGACTGTGTGATGCTGCCAACCCAACCAACTGTATTGCCCCTGTGAATGGCTACTTC
ATCAAGACCAACAACACCAGGATTGTGGATGAGTGGTCCTACACAGGCTCCTCCTTCTATGCCCCTGA
ACCAATCACCTCCCTGAACACCAAATATGTGGCTCCACAGGTGACCTACCAGAACATCAGCACCAACC
TGCCTCCTCCACTGCTGGGCAACAGCACAGGCATTGACTTCCAGGATGAACTGGATGAGTTCTTCAAG
AATGTGAGCACCAGCATCCCAAACTTTGGCTCCCTGACCCAGATAAACACCACCCTGCTGGACCTGAC
CTATGAGATGCTGTCCCTCCAACAGGTGGTGAAGGCTCTGAATGAGTCCTACATTGACCTGAAAGAAC
TGGGCAACTACACCTACTACAACAAGTGGCCATGGTACATCTGGCTGGGCTTCATCGCTGGCCTGGTG
GCCCTGGCGCTGTGCGTGTTCTTCATCCTGTGCTGCACCGGCTGCGGCACCAACTGCATGGGCAAGCT
GAAGTGCAACAGGTGCTGCTAAggatccgagctcggtaccaagcttaagtttaaaccgctgatcagcc
tcgactgtgccttctagttgccagccatctgttgtttgcccctcccccgtgccttccttgaccctgga
aggtgccactcccactgtcctttcctaataaaatgaggaaattgcatcgcattgtctgagtaggtgtc
attctattctggggggtggggtggggcaggacagcaagggggaggattgggaagacaatagcaggcat
gctggggatgcggtgggctctatggcttctgaggcggaaagaaccagctggggctctagggggtatcc
ccacgcgccctgtagcggcgcattaagcgcggcgggtgtggtggttacgcgcagcgtgaccgctacac
ttgccagcgccctagcgcccgctcctttcgctttcttcccttcctttctcgccacgttcgccggcttt
ccccgtcaagctctaaatcgggggctccctttagggttccgatttagtgctttacggcacctcgaccc
caaaaaacttgattagggtgatggttcacgtagtgggccatcgccctgatagacggtttttcgccctt
tgacgttggagtccacgttctttaatagtggactcttgttccaaactggaacaacactcaaccctatc
tcggtctattcttttgatttataagggattttgccgatttcggcctattggttaaaaaatgagctgat
ttaacaaaaatttaacgcgaattaattctgtggaatgtgtgtcagttagggtgtggaaagtccccagg
ctccccagcaggcagaagtatgcaaagcatgcatctcaattagtcagcaaccaggtgtggaaagtccc
caggctccccagcaggcagaagtatgcaaagcatgcatctcaattagtcagcaaccatagtcccgccc
ctaactccgcccatcccgcccctaactccgcccagttccgcccattctccgccccatggctgactaat
tttttttatttatgcagaggccgaggccgcctctgcctctgagctattccagaagtagtgaggaggct
tttttggaggcctaggcttttgcaaaaagctcccgggagcttgtatatccattttcggatctgatcaa
gagacaggatgaggatcgtttcgcatgattgaacaagatggattgcacgcaggttctccggccgcttg
ggtggagaggctattcggctatgactgggcacaacagacaatcggctgctctgatgccgccgtgttcc
ggctgtcagcgcaggggcgcccggttctttttgtcaagaccgacctgtccggtgccctgaatgaactg
caggacgaggcagcgcggctatcgtggctggccacgacgggcgttccttgcgcagctgtgctcgacgt
tgtcactgaagcgggaagggactggctgctattgggcgaagtgccggggcaggatctcctgtcatctc
accttgctcctgccgagaaagtatccatcatggctgatgcaatgcggcggctgcatacgcttgatccg
gctacctgcccattcgaccaccaagcgaaacatcgcatcgagcgagcacgtactcggatggaagccgg
tcttgtcgatcaggatgatctggacgaagagcatcaggggctcgcgccagccgaactgttcgccaggc
tcaaggcgcgcatgcccgacggcgaggatctcgtcgtgacccatggcgatgcctgcttgccgaatatc
atggtggaaaatggccgcttttctggattcatcgactgtggccggctgggtgtggcggaccgctatca
ggacatagcgttggctacccgtgatattgctgaagagcttggcggcgaatgggctgaccgcttcctcg
tgctttacggtatcgccgctcccgattcgcagcgcatcgccttctatcgccttcttgacgagttcttc
tgagcgggactctggggttcgaaatgaccgaccaagcgacgcccaacctgccatcacgagatttcgat
tccaccgccgccttctatgaaaggttgggcttcggaatcgttttccgggacgccggctggatgatcct
ccagcgcggggatctcatgctggagttcttcgcccaccccaacttgtttattgcagcttataatggtt
acaaataaagcaatagcatcacaaatttcacaaataaagcatttttttcactgcattctagttgtggt
ttgtccaaactcatcaatgtatcttatcatgtctgtataccgtcgacctctagctagagcttggcgta
atcatggtcatagctgtttcctgtgtgaaattgttatccgctcacaattccacacaacatacgagccg
gaagcataaagtgtaaagcctggggtgcctaatgagtgagctaactcacattaattgcgttgcgctca
ctgcccgctttccagtcgggaaacctgtcgtgccagctgcattaatgaatcggccaacgcgcggggag
aggcggtttgcgtattgggcgctcttccgcttcctcgctcactgactcgctgcgctcggtcgttcggc
tgcggcgagcggtatcagctcactcaaaggcggtaatacggttatccacagaatcaggggataacgca
ggaaagaacatgtgagcaaaaggccagcaaaaggccaggaaccgtaaaaaggccgcgttgctggcgtt
tttccataggctccgcccccctgacgagcatcacaaaaatcgacgctcaagtcagaggtggcgaaacc
cgacaggactataaagataccaggcgtttccccctggaagctccctcgtgcgctctcctgttccgacc
ctgccgcttaccggatacctgtccgcctttctcccttcgggaagcgtggcgctttctcatagctcacg
ctgtaggtatctcagttcggtgtaggtcgttcgctccaagctgggctgtgtgcacgaaccccccgttc
agcccgaccgctgcgccttatccggtaactatcgtcttgagtccaacccggtaagacacgacttatcg
ccactggcagcagccactggtaacaggattagcagagcgaggtatgtaggcggtgctacagagttctt
gaagtggtggcctaactacggctacactagaagaacagtatttggtatctgcgctctgctgaagccag
ttaccttcggaaaaagagttggtagctcttgatccggcaaacaaaccaccgctggtagcggtggtttt
tttgtttgcaagcagcagattacgcgcagaaaaaaaggatctcaagaagatcctttgatcttttctac
ggggtctgacgctcagtggaacgaaaactcacgttaagggattttggtcatgagattatcaaaaagga
tcttcacctagatccttttaaattaaaaatgaagttttaaatcaatctaaagtatatatgagtaaact
tggtctgacagttaccaatgcttaatcagtgaggcacctatctcagcgatctgtctatttcgttcatc
catagttgcctgactccccgtcgtgtagataactacgatacgggagggcttaccatctggccccagtg
ctgcaatgataccgcgagacccacgctcaccggctccagatttatcagcaataaaccagccagccgga
agggccgagcgcagaagtggtcctgcaactttatccgcctccatccagtctattaattgttgccggga
agctagagtaagtagttcgccagttaatagtttgcgcaacgttgttgccattgctacaggcatcgtgg
tgtcacgctcgtcgtttggtatggcttcattcagctccggttcccaacgatcaaggcgagttacatga
tcccccatgttgtgcaaaaaagcggttagctccttcggtcctccgatcgttgtcagaagtaagttggc
cgcagtgttatcactcatggttatggcagcactgcataattctcttactgtcatgccatccgtaagat
gcttttctgtgactggtgagtactcaaccaagtcattctgagaatagtgtatgcggcgaccgagttgc
tcttgcccggcgtcaatacgggataataccgcgccacatagcagaactttaaaagtgctcatcattgg
aaaacgttcttcggggcgaaaactctcaaggatcttaccgctgttgagatccagttcgatgtaaccca
ctcgtgcacccaactgatcttcagcatcttttactttcaccagcgtttctgggtgagcaaaaacagga
aggcaaaatgccgcaaaaaagggaataagggcgacacggaaatgttgaatactcatactcttcctttt
tcaatattattgaagcatttatcagggttattgtctcatgagcggatacatatttgaatgtatttaga
aaaataaacaaataggggttccgcgcacatttccccgaaaagtgccacctgacgtc
PVP40 SARS-COV-2 spike delta variant del 19
(SEQ ID NO: 45)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccATGTTTGTGTTCCTGGTGCTGCTGCCACTGGTGTCCAGCCAGTGTGTG
AACCTGAGGACCAGGACCCAACTTCCTCCTGCCTACACCAACTCCTTCACCAGGGGAGTCTACTACCC
TGACAAGGTGTTCAGGTCCTCTGTGCTGCACAGCACCCAGGACCTGTTCCTGCCATTCTTCAGCAATG
TGACCTGGTTCCATGCCATCCATGTGTCTGGCACCAATGGCACCAAGAGGTTTGACAACCCTGTGCTG
CCATTCAATGATGGAGTCTACTTTGCCAGCACAGAGAAGAGCAACATCATCAGGGGCTGGATTTTTGG
CACCACCCTGGACAGCAAGACCCAGTCCCTGCTGATTGTGAACAATGCCACCAATGTGGTGATTAAGG
TGTGTGAGTTCCAGTTCTGTAATGACCCATTCCTGGACGTCTACTACCACAAGAACAACAAGTCCTGG
ATGGAGTCTGGCGTCTACTCCTCTGCCAACAACTGTACCTTTGAATATGTGAGCCAACCATTCCTGAT
GGACTTGGAGGGCAAGCAGGGCAACTTCAAGAACCTGAGGGAGTTTGTGTTCAAGAACATTGATGGCT
ACTTCAAGATTTACAGCAAACACACACCAATCAACCTGGTGAGGGACCTGCCACAGGGCTTCTCTGCC
TTGGAACCACTGGTGGACCTGCCAATTGGCATCAACATCACCAGGTTCCAGACCCTGCTGGCTCTGCA
CAGGTCCTACCTGACACCTGGAGACTCCTCCTCTGGCTGGACAGCAGGAGCAGCAGCCTACTATGTGG
GCTACCTCCAACCAAGGACCTTCCTGCTGAAATACAATGAGAATGGCACCATCACAGATGCTGTGGAC
TGTGCCCTGGACCCACTGTCTGAGACCAAGTGTACCCTGAAATCCTTCACAGTGGAGAAGGGCATCTA
CCAGACCAGCAACTTCAGGGTCCAACCAACAGAGAGCATTGTGAGGTTTCCAAACATCACAAACCTGT
GTCCATTTGGAGAGGTGTTCAATGCCACCAGGTTTGCCTCTGTCTATGCCTGGAACAGGAAGAGGATT
AGCAACTGTGTGGCTGACTACTCTGTGCTCTACAACTCTGCCTCCTTCAGCACCTTCAAGTGTTATGG
AGTGAGCCCAACCAAACTGAATGACCTGTGTTTCACCAATGTCTATGCTGACTCCTTTGTGATTAGGG
GAGATGAGGTGAGACAGATTGCCCCTGGACAAACAGGCAAGATTGCTGACTACAACTACAAACTGCCT
GATGACTTCACAGGCTGTGTGATTGCCTGGAACAGCAACAACCTGGACAGCAAGGTGGGAGGCAACTA
CAACTACAGGTACAGACTGTTCAGGAAGAGCAACCTGAAACCATTTGAGAGGGACATCAGCACAGAGA
TTTACCAGGCTGGCAGCAAGCCATGTAATGGAGTGGAGGGCTTCAACTGTTACTTTCCACTCCAATCC
TATGGCTTCCAACCAACCAATGGAGTGGGCTACCAACCATACAGGGTGGTGGTGCTGTCCTTTGAACT
GCTCCATGCCCCTGCCACAGTGTGTGGACCAAAGAAGAGCACAAACCTGGTGAAGAACAAGTGTGTGA
ACTTCAACTTCAATGGACTGACAGGCACAGGAGTGCTGACAGAGAGCAACAAGAAGTTCCTGCCATTC
CAACAGTTTGGCAGGGACATTGCTGACACCACAGATGCTGTGAGGGACCCACAGACCTTGGAGATTCT
GGACATCACACCATGTTCCTTTGGAGGAGTGTCTGTGATTACACCTGGCACCAACACCAGCAACCAGG
TGGCTGTGCTCTACCAGGGCGTGAACTGTACTGAGGTGCCTGTGGCTATCCATGCTGACCAACTTACC
CCAACCTGGAGGGTCTACAGCACAGGCAGCAACGTGTTCCAGACCAGGGCTGGCTGTCTGATTGGAGC
AGAGCATGTGAACAACTCCTATGAGTGTGACATCCCAATTGGAGCAGGCATCTGTGCCTCCTACCAGA
CCCAGACCAACAGCAGGAGGAGGGCAAGGTCTGTGGCAAGCCAGAGCATCATTGCCTACACAATGAGT
CTGGGAGCAGAGAACTCTGTGGCTTACAGCAACAACAGCATTGCCATCCCAACCAACTTCACCATCTC
TGTGACCACAGAGATTCTGCCTGTGAGTATGACCAAGACCTCTGTGGACTGTACAATGTATATCTGTG
GAGACAGCACAGAGTGTAGCAACCTGCTGCTCCAATATGGCTCCTTCTGTACCCAACTTAACAGGGCT
CTGACAGGCATTGCTGTGGAACAGGACAAGAACACCCAGGAGGTGTTCGCCCAAGTGAAGCAGATTTA
CAAGACACCTCCAATCAAGGACTTTGGAGGCTTCAACTTCAGCCAGATTCTGCCTGACCCAAGCAAGC
CAAGCAAGAGGTCCTTCATTGAGGACCTGCTGTTCAACAAGGTGACCCTGGCTGATGCTGGCTTCATC
AAGCAATATGGAGACTGTCTGGGAGACATTGCCGCCAGGGACCTGATTTGTGCCCAGAAGTTCAATGG
ACTGACAGTGCTGCCTCCACTGCTGACAGATGAGATGATTGCCCAATACACCTCTGCCCTGCTGGCTG
GCACCATCACCTCTGGCTGGACCTTTGGAGCAGGAGCAGCCCTCCAAATCCCATTTGCTATGCAGATG
GCTTACAGGTTCAATGGCATTGGAGTGACCCAGAATGTGCTCTATGAGAACCAGAAACTGATTGCCAA
CCAGTTCAACTCTGCCATTGGCAAGATTCAGGACTCCCTGTCCAGCACAGCCTCTGCCCTGGGCAAGC
TCCAAAACGTGGTGAACCAGAATGCCCAGGCTCTGAACACCCTGGTGAAGCAACTTTCCAGCAACTTT
GGAGCCATCTCCTCTGTGCTGAATGACATCCTGAGCAGACTGGACAAGGTGGAGGCTGAGGTCCAGAT
TGACAGACTGATTACAGGCAGACTCCAATCCCTCCAAACCTATGTGACCCAACAACTTATCAGGGCTG
CTGAGATTAGGGCATCTGCCAACCTGGCTGCCACCAAGATGAGTGAGTGTGTGCTGGGACAAAGCAAG
AGGGTGGACTTCTGTGGCAAGGGCTACCACCTGATGAGTTTTCCACAGTCTGCCCCTCATGGAGTGGT
GTTCCTGCATGTGACCTATGTGCCTGCCCAGGAGAAGAACTTCACCACAGCCCCTGCCATCTGCCATG
ATGGCAAGGCTCACTTTCCAAGGGAGGGAGTGTTTGTGAGCAATGGCACCCACTGGTTTGTGACCCAG
AGGAACTTCTATGAACCACAGATTATCACCACAGACAACACCTTTGTGTCTGGCAACTGTGATGTGGT
GATTGGCATTGTGAACAACACAGTCTATGACCCACTCCAGCCTGAACTGGACTCCTTCAAGGAGGAAC
TGGACAAATACTTCAAGAACCACACCAGCCCTGATGTGGACCTGGGAGACATCTCTGGCATCAATGCC
TCTGTGGTGAACATCCAGAAGGAGATTGACAGACTGAATGAGGTGGCTAAGAACCTGAATGAGTCCCT
GATTGACCTCCAAGAACTGGGCAAATATGAACAATACATCAAGTGGCCATGGTACATCTGGCTGGGCT
TCATTGCTGGACTGATTGCCATTGTGATGGTGACCATAATGCTGTGTTGTATGACCTCCTGTTGTTCC
TGTCTGAAAGGCTGTTGTTCCTGTGGCTCCTGTTGTTGAggatccgagctcggtaccaagcttaagtt
taaaccgctgatcagcctcgactgtgccttctagttgccagccatctgttgtttgcccctcccccgtg
ccttccttgaccctggaaggtgccactcccactgtcctttcctaataaaatgaggaaattgcatcgca
ttgtctgagtaggtgtcattctattctggggggtggggtggggcaggacagcaagggggaggattggg
aagacaatagcaggcatgctggggatgcggtgggctctatggcttctgaggcggaaagaaccagctgg
ggctctagggggtatccccacgcgccctgtagcggcgcattaagcgcggcgggtgtggtggttacgcg
cagcgtgaccgctacacttgccagcgccctagcgcccgctcctttcgctttcttcccttcctttctcg
ccacgttcgccggctttccccgtcaagctctaaatcgggggctccctttagggttccgatttagtgct
ttacggcacctcgaccccaaaaaacttgattagggtgatggttcacgtagtgggccatcgccctgata
gacggtttttcgccctttgacgttggagtccacgttctttaatagtggactcttgttccaaactggaa
caacactcaaccctatctcggtctattcttttgatttataagggattttgccgatttcggcctattgg
ttaaaaaatgagctgatttaacaaaaatttaacgcgaattaattctgtggaatgtgtgtcagttaggg
tgtggaaagtccccaggctccccagcaggcagaagtatgcaaagcatgcatctcaattagtcagcaac
caggtgtggaaagtccccaggctccccagcaggcagaagtatgcaaagcatgcatctcaattagtcag
caaccatagtcccgcccctaactccgcccatcccgcccctaactccgcccagttccgcccattctccg
ccccatggctgactaattttttttatttatgcagaggccgaggccgcctctgcctctgagctattcca
gaagtagtgaggaggcttttttggaggcctaggcttttgcaaaaagctcccgggagcttgtatatcca
ttttcggatctgatcaagagacaggatgaggatcgtttcgcatgattgaacaagatggattgcacgca
ggttctccggccgcttgggtggagaggctattcggctatgactgggcacaacagacaatcggctgctc
tgatgccgccgtgttccggctgtcagcgcaggggcgcccggttctttttgtcaagaccgacctgtccg
gtgccctgaatgaactgcaggacgaggcagcgcggctatcgtggctggccacgacgggcgttccttgc
gcagctgtgctcgacgttgtcactgaagcgggaagggactggctgctattgggcgaagtgccggggca
ggatctcctgtcatctcaccttgctcctgccgagaaagtatccatcatggctgatgcaatgcggcggc
tgcatacgcttgatccggctacctgcccattcgaccaccaagcgaaacatcgcatcgagcgagcacgt
actcggatggaagccggtcttgtcgatcaggatgatctggacgaagagcatcaggggctcgcgccagc
cgaactgttcgccaggctcaaggcgcgcatgcccgacggcgaggatctcgtcgtgacccatggcgatg
cctgcttgccgaatatcatggtggaaaatggccgcttttctggattcatcgactgtggccggctgggt
gtggcggaccgctatcaggacatagcgttggctacccgtgatattgctgaagagcttggcggcgaatg
ggctgaccgcttcctcgtgctttacggtatcgccgctcccgattcgcagcgcatcgccttctatcgcc
ttcttgacgagttcttctgagcgggactctggggttcgaaatgaccgaccaagcgacgcccaacctgc
catcacgagatttcgattccaccgccgccttctatgaaaggttgggcttcggaatcgttttccgggac
gccggctggatgatcctccagcgcggggatctcatgctggagttcttcgcccaccccaacttgtttat
tgcagcttataatggttacaaataaagcaatagcatcacaaatttcacaaataaagcatttttttcac
tgcattctagttgtggtttgtccaaactcatcaatgtatcttatcatgtctgtataccgtcgacctct
agctagagcttggcgtaatcatggtcatagctgtttcctgtgtgaaattgttatccgctcacaattcc
acacaacatacgagccggaagcataaagtgtaaagcctggggtgcctaatgagtgagctaactcacat
taattgcgttgcgctcactgcccgctttccagtcgggaaacctgtcgtgccagctgcattaatgaatc
ggccaacgcgcggggagaggcggtttgcgtattgggcgctcttccgcttcctcgctcactgactcgct
gcgctcggtcgttcggctgcggcgagcggtatcagctcactcaaaggcggtaatacggttatccacag
aatcaggggataacgcaggaaagaacatgtgagcaaaaggccagcaaaaggccaggaaccgtaaaaag
gccgcgttgctggcgtttttccataggctccgcccccctgacgagcatcacaaaaatcgacgctcaag
tcagaggtggcgaaacccgacaggactataaagataccaggcgtttccccctggaagctccctcgtgc
gctctcctgttccgaccctgccgcttaccggatacctgtccgcctttctcccttcgggaagcgtggcg
ctttctcatagctcacgctgtaggtatctcagttcggtgtaggtcgttcgctccaagctgggctgtgt
gcacgaaccccccgttcagcccgaccgctgcgccttatccggtaactatcgtcttgagtccaacccgg
taagacacgacttatcgccactggcagcagccactggtaacaggattagcagagcgaggtatgtaggc
ggtgctacagagttcttgaagtggtggcctaactacggctacactagaagaacagtatttggtatctg
cgctctgctgaagccagttaccttcggaaaaagagttggtagctcttgatccggcaaacaaaccaccg
ctggtagcggtggtttttttgtttgcaagcagcagattacgcgcagaaaaaaaggatctcaagaagat
cctttgatcttttctacggggtctgacgctcagtggaacgaaaactcacgttaagggattttggtcat
gagattatcaaaaaggatcttcacctagatccttttaaattaaaaatgaagttttaaatcaatctaaa
gtatatatgagtaaacttggtctgacagttaccaatgcttaatcagtgaggcacctatctcagcgatc
tgtctatttcgttcatccatagttgcctgactccccgtcgtgtagataactacgatacgggagggctt
accatctggccccagtgctgcaatgataccgcgagacccacgctcaccggctccagatttatcagcaa
taaaccagccagccggaagggccgagcgcagaagtggtcctgcaactttatccgcctccatccagtct
attaattgttgccgggaagctagagtaagtagttcgccagttaatagtttgcgcaacgttgttgccat
tgctacaggcatcgtggtgtcacgctcgtcgtttggtatggcttcattcagctccggttcccaacgat
caaggcgagttacatgatcccccatgttgtgcaaaaaagcggttagctccttcggtcctccgatcgtt
gtcagaagtaagttggccgcagtgttatcactcatggttatggcagcactgcataattctcttactgt
catgccatccgtaagatgcttttctgtgactggtgagtactcaaccaagtcattctgagaatagtgta
tgcggcgaccgagttgctcttgcccggcgtcaatacgggataataccgcgccacatagcagaacttta
aaagtgctcatcattggaaaacgttcttcggggcgaaaactctcaaggatcttaccgctgttgagatc
cagttcgatgtaacccactcgtgcacccaactgatcttcagcatcttttactttcaccagcgtttctg
ggtgagcaaaaacaggaaggcaaaatgccgcaaaaaagggaataagggcgacacggaaatgttgaata
ctcatactcttcctttttcaatattattgaagcatttatcagggttattgtctcatgagcggatacat
atttgaatgtatttagaaaaataaacaaataggggttccgcgcacatttccccgaaaagtgccacctg
acgtc
SARS-COV-2 Delta Variant del 19 Spike
(SEQ ID NO: 47)
MFVFLVLLPLVSSQCVNLRTRTQLPPAYTNSFTRGVYYPDKVFRSSVLHSTQDLFLPFFSNVTWFHAI
HVSGTNGTKRFDNPVLPFNDGVYFASTEKSNIIRGWIFGTTLDSKTQSLLIVNNATNVVIKVCEFQFC
NDPFLDVYYHKNNKSWMESGVYSSANNCTFEYVSQPFLMDLEGKQGNEKNLREFVEKNIDGYFKIYSK
HTPINLVRDLPQGFSALEPLVDLPIGINITRFQTLLALHRSYLTPGDSSSGWTAGAAAYYVGYLQPRT
FLLKYNENGTITDAVDCALDPLSETKCTLKSFTVEKGIYQTSNERVQPTESIVRFPNITNLCPFGEVE
NATRFASVYAWNRKRISNCVADYSVLYNSASFSTFKCYGVSPTKLNDLCFTNVYADSEVIRGDEVRQI
APGQTGKIADYNYKLPDDFTGCVIAWNSNNLDSKVGGNYNYRYRLFRKSNLKPFERDISTEIYQAGSK
PCNGVEGENCYFPLQSYGFQPTNGVGYQPYRVVVLSFELLHAPATVCGPKKSTNLVKNKCVNFNENGL
TGTGVLTESNKKELPFQQFGRDIADTTDAVRDPQTLEILDITPCSFGGVSVITPGTNTSNQVAVLYQG
VNCTEVPVAIHADQLTPTWRVYSTGSNVFQTRAGCLIGAEHVNNSYECDIPIGAGICASYQTQTNSRR
RARSVASQSIIAYTMSLGAENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGENESQILPDPSKPSKRSFI
EDLLENKVTLADAGFIKQYGDCLGDIAARDLICAQKENGLTVLPPLLTDEMIAQYTSALLAGTITSGW
TFGAGAALQIPFAMQMAYRENGIGVTQNVLYENQKLIANQFNSAIGKIQDSLSSTASALGKLONVVNQ
NAQALNTLVKQLSSNFGAISSVLNDILSRLDKVEAEVQIDRLITGRLQSLQTYVTQQLIRAAEIRASA
NLAATKMSECVLGQSKRVDFCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWPWYIWLGFIAGLIA
IVMVTIMLCCMTSCCSCLKGCCSCGSCC*
MERS-COV Spike DeltaC-16
(SEQ ID NO: 48)
MIHSVFLLMFLLTPTESYVDVGPDSVKSACIEVDIQQTFFDKTWPRPIDVSKADGIIYPQGRTYSNIT
ITYQGLFPYQGDHGDMYVYSAGHATGTTPQKLFVANYSQDVKQFANGFVVRIGAAANSTGTVIISPST
SATIRKIYPAFMLGSSVGNFSDGKMGRFFNHTLVLLPDGCGTLLRAFYCILEPRSGNHCPAGNSYTSE
ATYHTPATDCSDGNYNRNASLNSFKEYFNLRNCTFMYTYNITEDEILEWFGITQTAQGVHLESSRYVD
LYGGNMFQFATLPVYDTIKYYSIIPHSIRSIQSDRKAWAAFYVYKLQPLTELLDESVDGYIRRAIDCG
FNDLSQLHCSYESFDVESGVYSVSSFEAKPSGSVVEQAEGVECDESPLLSGTPPQVYNEKRLVETNCN
YNLTKLLSLESVNDFTCSQISPAAIASNCYSSLILDYFSYPLSMKSDLSVSSAGPISQFNYKQSESNP
TCLILATVPHNLTTITKPLKYSYINKCSRLLSDDRTEVPQLVNANQYSPCVSIVPSTVWEDGDYYRKQ
LSPLEGGGWLVASGSTVAMTEQLQMGFGITVQYGTDTNSVCPKLEFANDTKIASQLGNCVEYSLYGVS
GRGVFQNCTAVGVRQQRFVYDAYQNLVGYYSDDGNYYCLRACVSVPVSVIYDKETKTHATLFGSVACE
HISSTMSQYSRSTRSMLKRRDSTYGPLQTPVGCVLGLVNSSLFVEDCKLPLGQSLCALPDTPSTLTPR
SVRSVPGEMRLASIAFNHPIQVDQLNSSYFKLSIPTNFSFGVTQEYIQTTIQKVTVDCKQYVCNGFQK
CEQLLREYGQFCSKINQALHGANLRQDDSVRNLFASVKSSQSSPIIPGFGGDENLTLLEPVSISTGSR
SARSAIEDLLEDKVTIADPGYMQGYDDCMQQGPASARDLICAQYVAGYKVLPPLMDVNMEAAYTSSLL
GSIAGVGWTAGLSSFAAIPFAQSIFYRLNGVGITQQVLSENQKLIANKENQALGAMQTGFTTTNEAFQ
KVQDAVNNNAQALSKLASELSNTFGAISASIGDIIQRLDVLEQDAQIDRLINGRLTTLNAFVAQQLVR
SESAALSAQLAKDKVNECVKAQSKRSGFCGQGTHIVSFVVNAPNGLYFMHVGYYPSNHIEVVSAYGLC
DAANPTNCIAPVNGYFIKTNNTRIVDEWSYTGSSFYAPEPITSLNTKYVAPQVTYQNISTNLPPPLLG
NSTGIDFQDELDEFFKNVSTSIPNFGSLTQINTTLLDLTYEMLSLQQVVKALNESYIDLKELGNYTYY
NKWPWYIWLGFIAGLVALALCVFFILCCTGCGTNCMGKLKCNRCC*
SARS-COV Spike Delta C-19
(SEQ ID NO: 49)
MFIFLLELTLTSGSDLDRCTTFDDVQAPNYTQHTSSMRGVYYPDEIFRSDTLYLTQDLFLPFYSNVTG
FHTINHTEDNPVIPFKDGIYFAATEKSNVVRGWVFGSTMNNKSQSVIIINNSTNVVIRACNFELCDNP
FFAVSKPMGTQTHTMIFDNAFNCTFEYISDAFSLDVSEKSGNFKHLREFVFKNKDGFLYVYKGYQPID
VVRDLPSGFNTLKPIFKLPLGINITNFRAILTAFSPAQDTWGTSAAAYFVGYLKPTTEMLKYDENGTI
TDAVDCSQNPLAELKCSVKSFEIDKGIYQTSNFRVVPSGDVVRFPNITNLCPFGEVENATKFPSVYAW
ERKKISNCVADYSVLYNSTFFSTFKCYGVSATKLNDLCFSNVYADSFVVKGDDVRQIAPGQTGVIADY
NYKLPDDEMGCVLAWNTRNIDATSTGNYNYKYRYLRHGKLRPFERDISNVPFSPDGKPCTPPALNCYW
PLNDYGFYTTTGIGYQPYRVVVLSFELLNAPATVCGPKLSTDLIKNQCVNFNFNGLTGTGVLTPSSKR
FQPFQQFGRDVSDFTDSVRDPKTSEILDISPCSFGGVSVITPGTNASSEVAVLYQDVNCTDVSTAIHA
DQLTPAWRIYSTGNNVFQTQAGCLIGAEHVDTSYECDIPIGAGICASYHTVSLLRSTSQKSIVAYTMS
LGADSSIAYSNNTIAIPTNFSISITTEVMPVSMAKTSVDCNMYICGDSTECANLLLQYGSFCTQLNRA
LSGIAAEQDRNTREVFAQVKQMYKTPTLKYFGGFNFSQILPDPLKPTKRSFIEDLLENKVTLADAGEM
KQYGECLGDINARDLICAQKENGLTVLPPLLTDDMIAAYTAALVSGTATAGWTFGAGAALQIPFAMQM
AYRENGIGVTQNVLYENQKQIANQFNKAISQIQESLTTTSTALGKLQDVVNQNAQALNTLVKQLSSNF
GAISSVLNDILSRLDKVEAEVQIDRLITGRLQSLQTYVTQQLIRAAEIRASANLAATKMSECVLGQSK
RVDFCGKGYHLMSFPQAAPHGVVFLHVTYVPSQERNFTTAPAICHEGKAYFPREGVFVENGTSWFITQ
RNFFSPQIITTDNTFVSGNCDVVIGIINNTVYDPLQPELDSFKEELDKYFKNHTSPDVDLGDISGINA
SVVNIQKEIDRLNEVAKNLNESLIDLQELGKYEQYIKWPWYVWLGFIAGLIAIVMVTILLCCMTSCCS
CLKGACSCGSCC*
SARS-COV-2 Spike Hexapro
(SEQ ID NO: 50)
MFVFLVLLPLVSSQCVNLRTRTQLPPAYTNSFTRGVYYPDKVFRSSVLHSTQDLFLPFFSNVTWFHAI
HVSGTNGTKRFDNPVLPENDGVYFASTEKSNIIRGWIFGTTLDSKTQSLLIVNNATNVVIKVCEFQFC
NDPFLDVYYHKNNKSWMESGVYSSANNCTFEYVSQPFLMDLEGKQGNFKNLREFVFKNIDGYFKIYSK
HTPINLVRDLPQGFSALEPLVDLPIGINITRFQTLLALHRSYLTPGDSSSGWTAGAAAYYVGYLQPRT
FLLKYNENGTITDAVDCALDPLSETKCTLKSFTVEKGIYQTSNERVQPTESIVRFPNITNLCPFGEVE
NATRFASVYAWNRKRISNCVADYSVLYNSASFSTFKCYGVSPTKLNDLCFTNVYADSEVIRGDEVRQI
APGQTGKIADYNYKLPDDFTGCVIAWNSNNLDSKVGGNYNYRYRLFRKSNLKPFERDISTEIYQAGSK
PCNGVEGENCYFPLQSYGFQPTNGVGYQPYRVVVLSFELLHAPATVCGPKKSTNLVKNKCVNENENGL
TGTGVLTESNKKELPFQQFGRDIADTTDAVRDPQTLEILDITPCSFGGVSVITPGTNTSNQVAVLYQG
VNCTEVPVAIHADQLTPTWRVYSTGSNVFQTRAGCLIGAEHVNNSYECDIPIGAGICASYQTQTNSRG
SASSVASQSIIAYTMSLGAENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGENFSQILPDPSKPSKRSPI
EDLLENKVTLADAGFIKQYGDCLGDIAARDLICAQKENGLTVLPPLLTDEMIAQYTSALLAGTITSGW
TFGAGPALQIPFPMQMAYRENGIGVTQNVLYENQKLIANQFNSAIGKIQDSLSSTPSALGKLONVVNQ
NAQALNTLVKQLSSNFGAISSVLNDILSRLDPPEAEVQIDRLITGRLQSLQTYVTQQLIRAAEIRASA
NLAATKMSECVLGQSKRVDFCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWPWYIWLGFIAGLIA
IVMVTIMLCCMTSCCSCLKGCCSCGSCCKEDEDDSEPVLKGVKLHYT*
MERS-COV Spike Hexapro
(SEQ ID NO: 51)
MIHSVFLLMFLLTPTESYVDVGPDSVKSACIEVDIQQTFFDKTWPRPIDVSKADGIIYPQGRTYSNIT
ITYQGLFPYQGDHGDMYVYSAGHATGTTPQKLFVANYSQDVKQFANGFVVRIGAAANSTGTVIISPST
SATIRKIYPAFMLGSSVGNFSDGKMGRFFNHTLVLLPDGCGTLLRAFYCILEPRSGNHCPAGNSYTSE
ATYHTPATDCSDGNYNRNASLNSFKEYFNLRNCTFMYTYNITEDEILEWFGITQTAQGVHLESSRYVD
LYGGNMFQFATLPVYDTIKYYSIIPHSIRSIQSDRKAWAAFYVYKLQPLTELLDESVDGYIRRAIDCG
FNDLSQLHCSYESFDVESGVYSVSSFEAKPSGSVVEQAEGVECDESPLLSGTPPQVYNFKRLVETNCN
YNLTKLLSLFSVNDFTCSQISPAAIASNCYSSLILDYFSYPLSMKSDLSVSSAGPISQFNYKQSESNP
TCLILATVPHNLTTITKPLKYSYINKCSRLLSDDRTEVPQLVNANQYSPCVSIVPSTVWEDGDYYRKQ
LSPLEGGGWLVASGSTVAMTEQLQMGFGITVQYGTDTNSVCPKLEFANDTKIASQLGNCVEYSLYGVS
GRGVFQNCTAVGVRQQREVYDAYQNLVGYYSDDGNYYCLRACVSVPVSVIYDKETKTHATLEGSVACE
HISSTMSQYSRSTRSMLKRRDSTYGPLQTPVGCVLGLVNSSLFVEDCKLPLGQSLCALPDTPSTLTPR
SVRSVPGEMRLASIAFNHPIQVDQLNSSYFKLSIPTNFSFGVTQEYIQTTIQKVTVDCKQYVCNGFQK
CEQLLREYGQFCSKINQALHGANLRQDDSVRNLFASVKSSQSSPIIPGEGGDENLTLLE
SARS-COV Spike Hexapro
(SEQ ID NO: 52)
MFIFLLFLTLTSGSDLDRCTTFDDVQAPNYTQHTSSMRGVYYPDEIFRSDTLYLTQDLFLPFYSNVTG
FHTINHTEDNPVIPFKDGIYFAATEKSNVVRGWVFGSTMNNKSQSVIIINNSTNVVIRACNFELCDNP
FFAVSKPMGTQTHTMIFDNAENCTFEYISDAFSLDVSEKSGNFKHLREFVEKNKDGFLYVYKGYQPID
VVRDLPSGFNTLKPIFKLPLGINITNFRAILTAFSPAQDTWGTSAAAYFVGYLKPTTFMLKYDENGTI
TDAVDCSQNPLAELKCSVKSFEIDKGIYQTSNFRVVPSGDVVRFPNITNLCPFGEVENATKEPSVYAW
ERKKISNCVADYSVLYNSTFFSTFKCYGVSATKLNDLCFSNVYADSFVVKGDDVRQIAPGQTGVIADY
NYKLPDDEMGCVLAWNTRNIDATSTGNYNYKYRYLRHGKLRPFERDISNVPFSPDGKPCTPPALNCYW
PLNDYGFYTTTGIGYQPYRVVVLSFELLNAPATVCGPKLSTDLIKNQCVNFNFNGLTGTGVLTPSSKR
FQPFQQFGRDVSDFTDSVRDPKTSEILDISPCSFGGVSVITPGTNASSEVAVLYQDVNCTDVSTAIHA
DQLTPAWRIYSTGNNVFQTQAGCLIGAEHVDTSYECDIPIGAGICASYHTVSLLRSTSQKSIVAYTMS
LGADSSIAYSNNTIAIPTNFSISITTEVMPVSMAKTSVDCNMYICGDSTECANLLLQYGSFCTQLNRA
LSGIAAEQDRNTREVFAQVKQMYKTPTLKYFGGFNFSQILPDPLKPTKRSPIEDLLENKVTLADAGEM
KQYGECLGDINARDLICAQKENGLTVLPPLLTDDMIAAYTAALVSGTATAGWTFGAGPALQIPFPMQM
AYRENGIGVTQNVLYENQKQIANQFNKAISQIQESLTTTPTALGKLQDVVNQNAQALNTLVKQLSSNF
GAISSVLNDILSRLDPPEAEVQIDRLITGRLQSLQTYVTQQLIRAAEIRASANLAATKMSECVLGQSK
RVDFCGKGYHLMSFPQAAPHGVVFLHVTYVPSQERNFTTAPAICHEGKAYFPREGVEVENGTSWFITQ
RNFFSPQIITTDNTFVSGNCDVVIGIINNTVYDPLQPELDSFKEELDKYFKNHTSPDVDLGDISGINA
SVVNIQKEIDRLNEVAKNLNESLIDLQELGKYEQYIKWPWYVWLGFIAGLIAIVMVTILLCCMTSCCS
CLKGACSCGSCCKFDEDDSEPVLKGVKLHYT*
SARS-COV-2 Spike Hexapro (pVP31b)
(SEQ ID NO: 53)
MFVFLVLLPLVSSQCVNLTTRTQLPPAYTNSFTRGVYYPDKVFRSSVLHSTQDLFLPFFSNVTWFHAI
HVSGTNGTKRFDNPVLPENDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNEKNLREFVEKNIDGYFKIY
SKHTPINLVRDLPQGFSALEPLVDLPIGINITRFQTLLALHRSYLTPGDSSSGWTAGAAAYYVGYLQP
RTFLLKYNENGTITDAVDCALDPLSETKCTLKSFTVEKGIYQTSNERVQPTESIVRFPNITNLCPFGE
VENATRFASVYAWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKSNLKPFERDISTEIYQAG
STPCNGVEGENCYFPLQSYGFQPTNGVGYQPYRVVVLSFELLHAPATVCGPKKSTNLVKNKCVNENEN
GLTGTGVLTESNKKELPFQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECDIPIGAGICASYQTQTNS
PGSASSVASQSIIAYTMSLGAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDEGGENFSQILPDPSKPSKRS
PIEDLLFNKVTLADAGFIKQYGDCLGDIAARDLICAQKENGLTVLPPLLTDEMIAQYTSALLAGTITS
GWTFGAGPALQIPFPMQMAYRENGIGVTQNVLYENQKLIANQFNSAIGKIQDSLSSTPSALGKLQDVV
NQNAQALNTLVKQLSSNFGAISSVLNDILSRLDPPEAEVQIDRLITGRLQSLQTYVTQQLIRAAEIRA
SANLAATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQYIKWPWYIWLGFIAGL
IAIVMVTIMLCCMTSCCSCLKGCCSCGSCCKEDEDDSEPVLKGVKLHYT*
SARS-COV-2 Spike Delta C-19 (pVP24)
(SEQ ID NO: 54)
MFVFLVLLPLVSSQCVNLTTRTQLPPAYTNSFTRGVYYPDKVFRSSVLHSTQDLFLPFFSNVTWFHAI
HVSGTNGTKRFDNPVLPENDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNEKNLREFVEKNIDGYFKIY
SKHTPINLVRDLPQGESALEPLVDLPIGINITRFQTLLALHRSYLTPGDSSSGWTAGAAAYYVGYLQP
RTFLLKYNENGTITDAVDCALDPLSETKCTLKSFTVEKGIYQTSNERVQPTESIVRFPNITNLCPFGE
VENATRFASVYAWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLERKSNLKPFERDISTEIYQAG
STPCNGVEGENCYFPLQSYGFQPTNGVGYQPYRVVVLSFELLHAPATVCGPKKSTNLVKNKCVNENEN
GLTGTGVLTESNKKELPFQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECDIPIGAGICASYQTQTNS
PRRARSVASQSIIAYTMSLGAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDFGGENFSQILPDPSKPSKRS
FIEDLLENKVTLADAGFIKQYGDCLGDIAARDLICAQKENGLTVLPPLLTDEMIAQYTSALLAGTITS
GWTFGAGAALQIPFAMQMAYRENGIGVTQNVLYENQKLIANQFNSAIGKIQDSLSSTASALGKLQDVV
NQNAQALNTLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQYIKWPWYIWLGFIAGL
IAIVMVTIMLCCMTSCCSCLKGCCSCGSCC*

### Results

### Design and Biophysical Characterization of Triplex Coronavirus Vaccine Against SARS-CoV-2, SARS-CoV and MERS-CoV

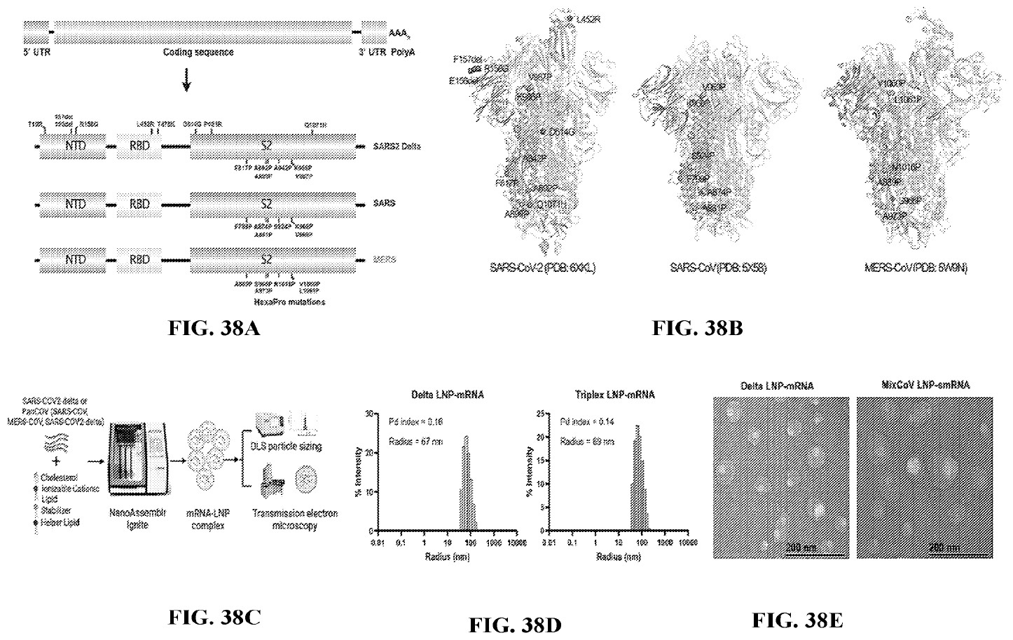

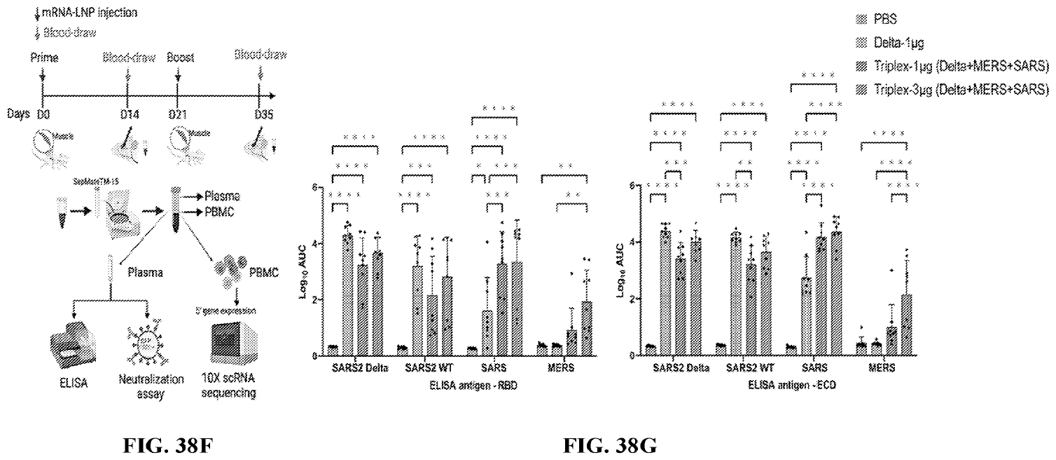

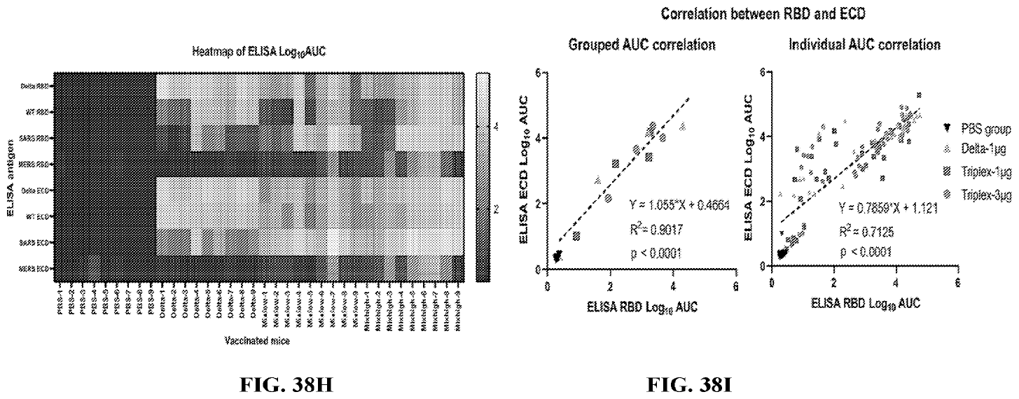

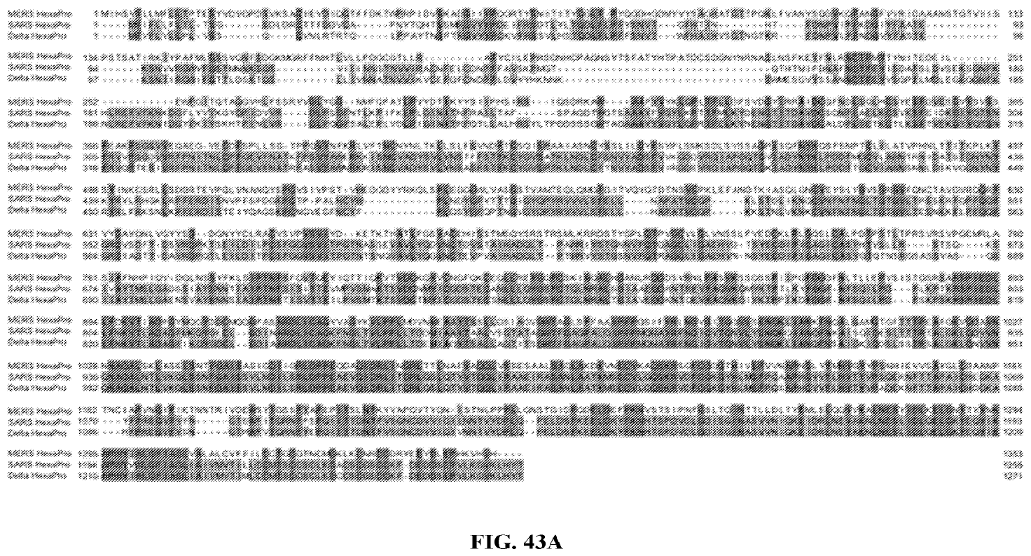

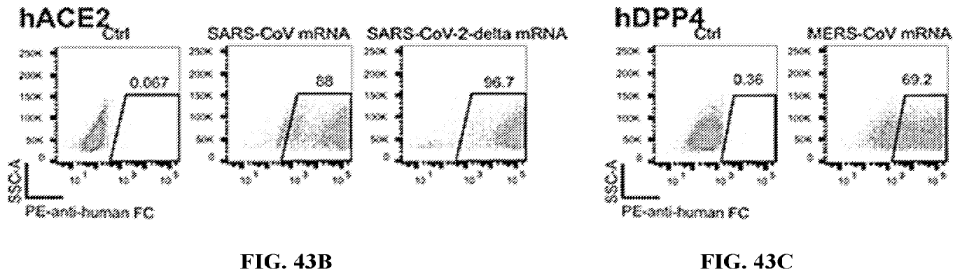

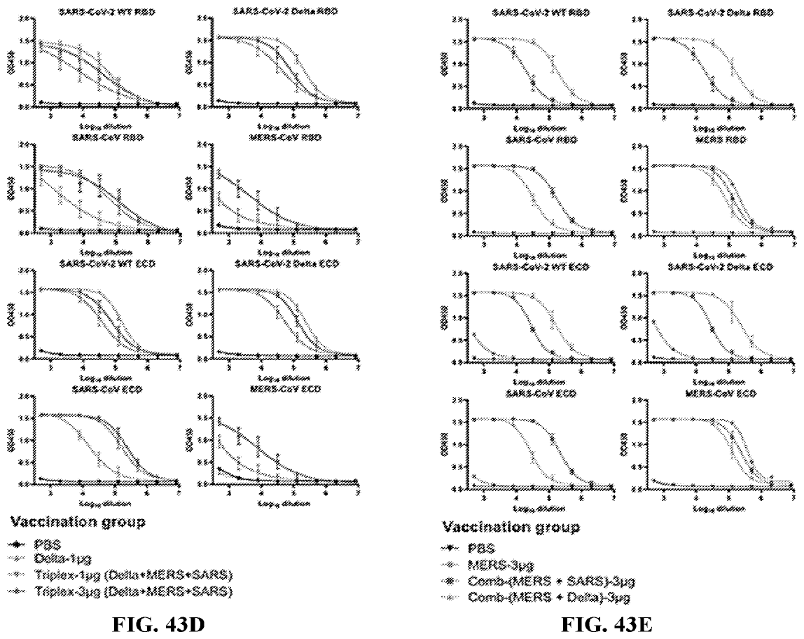

Vaccine candidate constructs encoding full-length spike mRNA of SARS-CoV-2 (labeled as SARS2 for short) Delta variant (Delta), SARS-CoV (SARS) and MERS-CoV (MERS) were first designed (FIGS. 38A-38B; FIG. 43A). Each construct contains a 5′untranslated region (UTR), an open reading frame (ORF), a 3′UTR and a polyA signal. The ORFs encode full-length spikes of defined species (SARS2, SARS and MERS), in which 6 additional proline mutations (HexaPro) were introduced in the S2 domain of the respective species (FIGS. 38A-38B), based on the homologous amino acid positions of SARS-CoV-2, to improve expression and stable prefusion state of spikes (Hsieh et al., 2020). The Delta construct ORF encodes the spike of SARS-CoV-2 Delta variant, which has nine mutations (T19R, 156del, 157del, R158G, L452R, T478K, D614G, P681R, and Q1071H) as compared to the original “wildtype” virus (WT, WA-1 or WA1) virus (FIGS. 1A-1B). We tested each of these mRNA constructs and showed that they all successfully generate functional protein upon introduction into mammalian cells, as evident by surface binding to the cognate human receptors, hACE2 for SARS-CoV and SARS-CoV-2, and hDPP4 for MERS, respectively (FIGS. 43B-43C).

To multiplex these constructs, an equal-mass mixture of spike mRNA of Delta, SARS and MERS, was prepared which were then encapsulated by lipid nanoparticles on a microfluidics instrument, to generate a triplex LNP-mRNA formulation of vaccine candidate (termed as Triplex or MixCoV, interchangeable aliases) (FIG. 38C). A Delta singlet LNP-mRNA for testing in parallel was also prepared. The size and homogeneity of assembled LNPs were evaluated by dynamic light scatter and transmission electron microscope (FIGS. 38D-E). The Delta LNP-mRNA and Triplex LNP-mRNA showed monodispersed size distribution with averaged radius of 70+3.8 nm and 71+3.6 nm, and polydispersity indices of 0.160 and 0.157, respectively. To evaluate the immunogenicity of Delta and Triplex LNP-mRNA vaccines, C57BL/6Ncr (B6) mice were immunized intramuscularly with two doses (prime and boost) of 1 μg Delta LNP-mRNA, 1 μg or 3 μg (total mRNA mass) Triplex LNP-mRNA, three weeks apart (FIG. 38F). The peripheral blood mononuclear cells (PBMCs) and plasma were collected two weeks post boost. The mice humoral response including binding and neutralizing antibody response against spike antigens were examined by ELISA and neutralization assays using collected plasma samples. Single cell RNA-sequencing (scRNA-seq) was performed to profile the systemic immune repertoires and their respective transcriptomics in vaccinated animals (FIG. 38F).

### Immune Responses to Triplex Coronavirus LNP-mRNA Vaccination Against SARS2, SARS and MERS

Compared to the PBS control group, the 1 μg Delta LNP-mRNA, 1 μg and 3 μg Triplex LNP-mRNA all elicited potent antibody response, as seen in the high post-boost binding antibody titers against both RBD and ECD of Delta, WT and SARS spikes (FIGS. 1G-1H). Among the three vaccination groups, only 3 μg Triplex LNP-mRNA significantly boosted mice immunity to MERS antigens (FIGS. 38G-38H and FIG. 43). As the Delta and Triplex vaccines used the Delta variant as spike antigen, their responses to Delta ELISA antigen were found slightly higher than WT antigen (FIG. 38G and FIG. 43). Despite of the lack of SARS spike antigen in the vaccine, the Delta LNP-mRNA induced antibodies that cross-react with SARS spike, but not MERS spike (FIG. 38G and FIG. 43), consistent with the respective degree of homology between these species (FIG. 43A). The titers are at similarly high level between the 1 μg and 3 μg Triplex groups for SARS and SARS2 spikes (FIG. 38G), while there is a trend of dose-dependent increase although statistically insignificant (FIG. 43). Compared to those of MixCoV 1 μg or 3 μg groups, SARS-binding antibody titer in 1 μg Delta LNP-mRNA group was significantly lower. A dose-dependent increase trend of antibody titers was observed for MERS spike in the two triplex vaccination groups (FIG. 38G). Within the Triplex groups, it is worth noting that antibody titer against MERS was 10-20 fold lower than that against SARS-CoV or SARS-CoV-2. Considering Delta spike mRNA at the same dose, mice in the 1 μg Delta and 3 μg Triplex (that also contains 1 μg Delta mRNA) groups showed similar titers of antibodies against SARS2 WT and Delta spikes, although an insignificant trend of lower titers was observed in the 3 μg Triplex mice (FIG. 38G and FIG. 43). Both ECD and RBD ELISA antigen panels showed highly correlated results among four spike types used (FIG. 381). In addition, a subset of animals showed relatively higher titer to ECD than RBD (FIG. 38I, off-the-diagonal data points), potentially due to the additional antibody reactivity outside RBD in those animals.

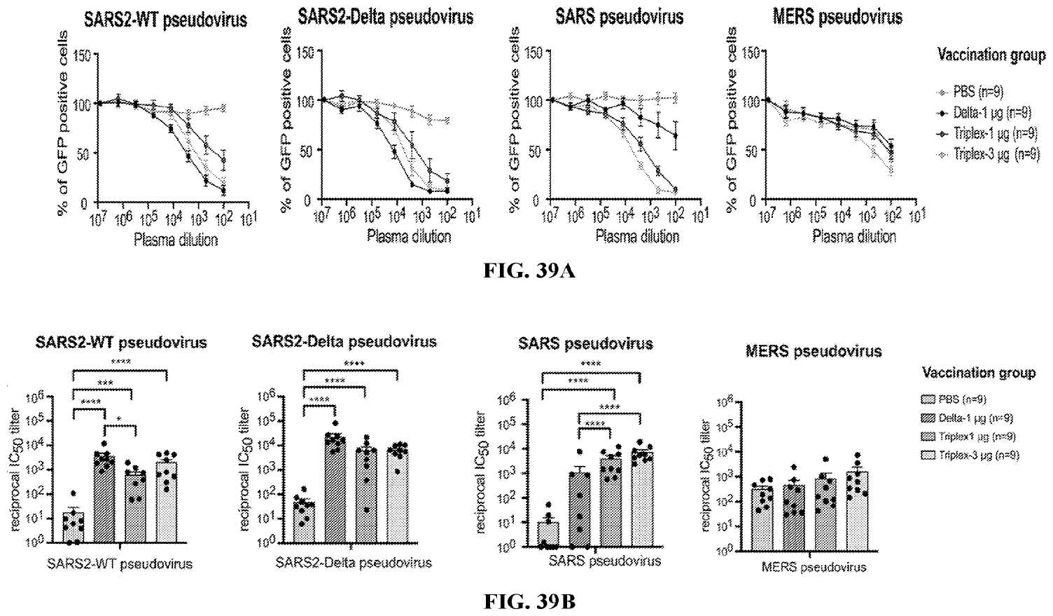

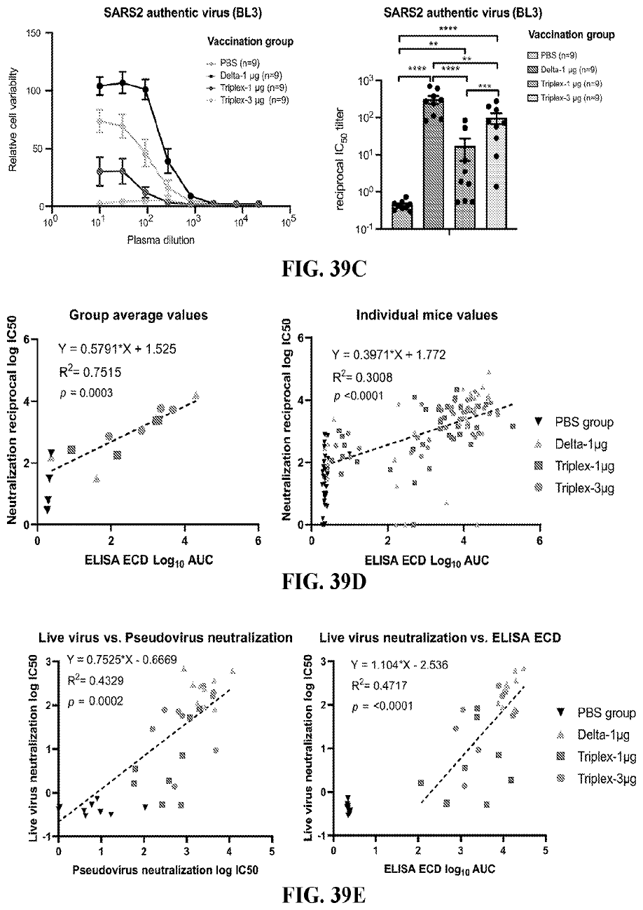

Subsequent studies then went on to examine the neutralizing antibody response in the pseudo-virus assay. All three Delta and Triplex-CoV LNP-mRNA vaccines induced marked increase in neutralizing antibodies against SARS2 WT/WA-1, Delta and SARS pseudoviruses (FIGS. 39A-B), which mimicked the overall titer landscape of binding antibodies in ELISA. All three LNP-mRNA groups (1 μg Delta, 1 μg and 3 μg Triplex-CoV) elicited potent neutralization activity against SARS2 Delta in the plasma of the vaccinated animals (FIGS. 39A-39B). In addition, both Triplex-CoV LNP-mRNA groups (1 μg and 3 μg Triplex-CoV) elicited potent neutralization activity against SARS in the plasma samples of the vaccinated animals (FIGS. 39A-39B). Despite the lack of SARS mRNA, the Delta alone group of LNP-mRNA also elicited substantial level of anti-SARS neutralization antibody response in a fraction of animals (4/9 above background) with high variation, although significantly lower than those of the Triplex-CoV groups (FIGS. 39A-39B), again potentially due to the similarity between the two species (FIG. 43A). The significantly higher antibody titer against SARS and higher robustness highlighted superior SARS protection efficacy of Triplex-CoV vaccine than Delta vaccine alone against SARS. Moderate neutralization activity against MERS was observed at this dosing scheme, with PBS group showing relatively high background level of neutralization (FIGS. 39A-B). Similar to ELISA, the neutralization activities are at similarly high level between the 1 μg and 3 μg Triplex-CoV groups for SARS and SARS2 spikes (FIGS. 39A-39B), while there is a trend of dose-dependent increase although statistically insignificant. We validated the neutralization against authentic virus of SARS-CoV-2 in a Biosafety Level 3 (BL3) setting, where the plasma samples from all three LNP-mRNA groups (1 μg Delta, 1 μg and 3 μg Triplex-CoV) showed significant neutralization activity (FIG. 39C). Similar to the observations in BL2 pseudovirus assay, the Triplex vaccination at both doses showed a lower level of neutralization against SARS2 as compared to Delta vaccination alone (FIG. 39C). Overall, antibody binding against ECD (ELISA) significantly correlated with neutralization activities for all groups or all mice, among spikes and pseudoviruses tested (FIG. 39D). Consistent with various previous reports, neutralization activity against authentic virus (BL3) significantly correlated with neutralization activities against pseudovirus (BL2), and correlated with binding antibody titers by ELISA (FIG. 39E).

### Immune Responses to Duplex Coronavirus LNP-mRNA Vaccination Centering on MERS

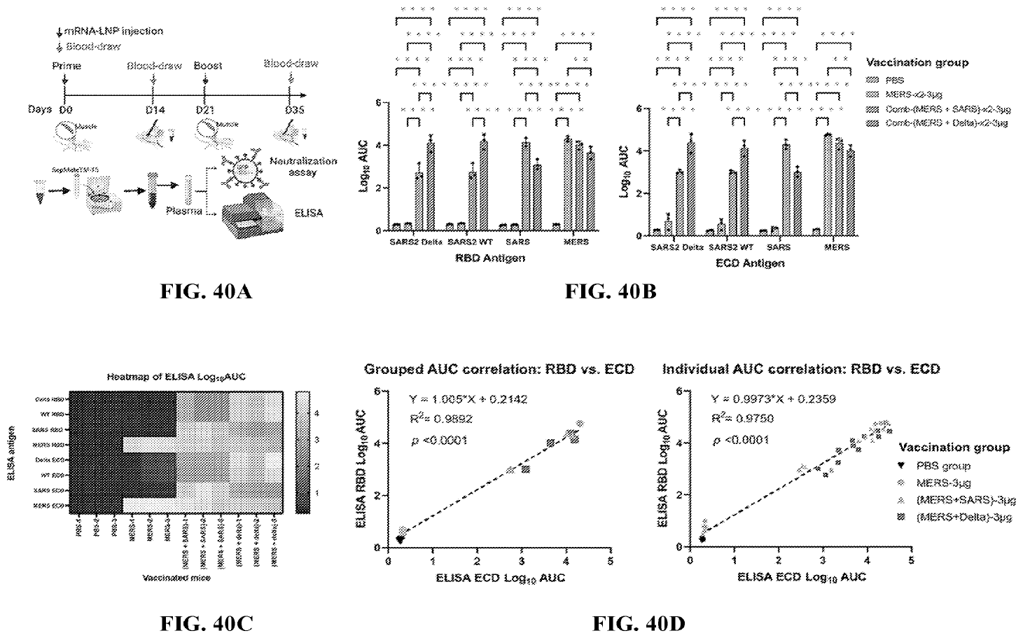

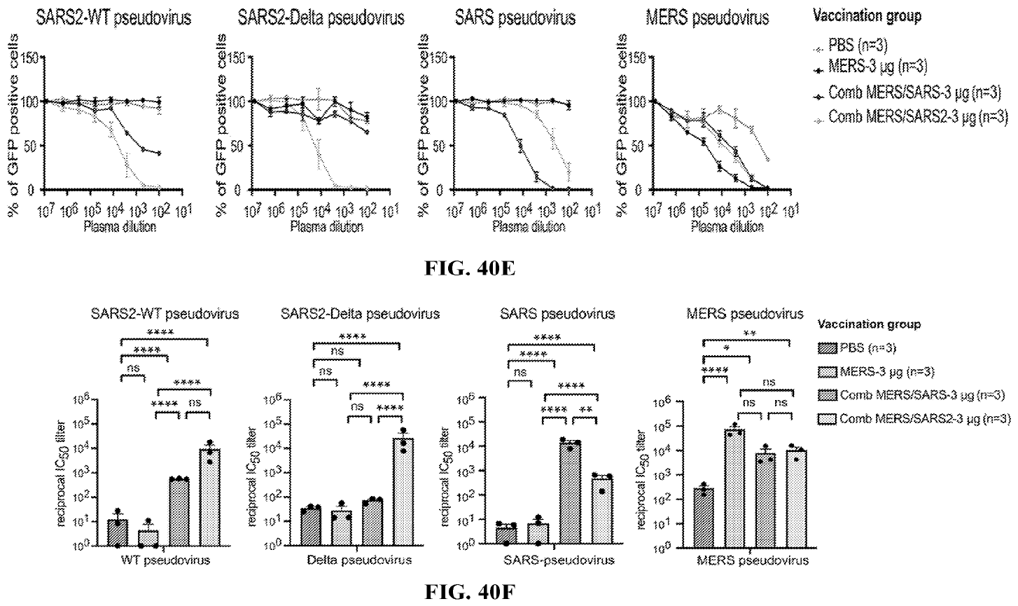

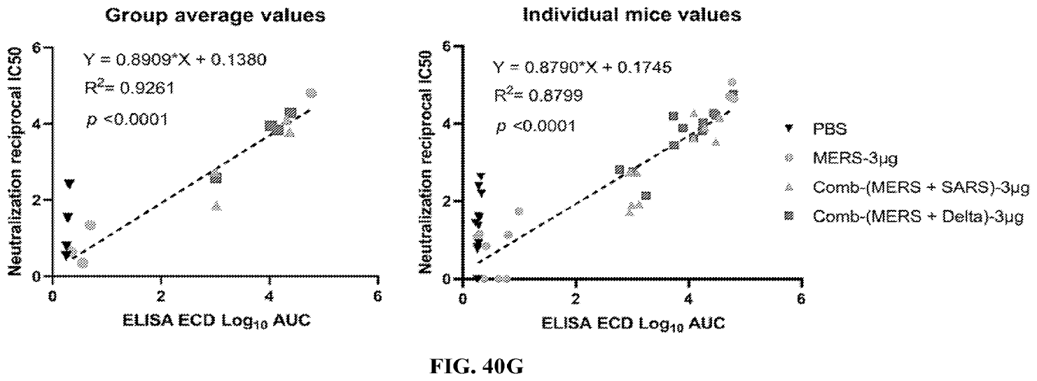

As the levels of Triplex-CoV-induced MERS binding and neutralizing antibodies were relatively lower than those of SARS and SARS2 in the Triplex-CoV groups (FIGS. 38 and 39), we sought to test single and duplex vaccination schemes for MERS LNP-mRNA (FIG. 40A). A proline mutation engineered prefusion MERS-CoV spike antigen has been previously generated and purified, which provided the basis for a MERS mRNA vaccine in clinical development. To test how the MERS HexaPro spike LNP-mRNA can work in combination with SARS2 Delta or SARS LNP-mRNA as duplex vaccines, we designed a duplex vaccine experiment for MERS LNP-mRNA. In the single vaccine scheme, we used the MERS LNP-mRNA alone (MERS Singlet). In the Duplex vaccine schemes, we mixed the MERS plus SARS, or MERS plus SARS2 Delta mRNAs, for the formulation of LNP-mRNAs (MERS Duplexes).

Mice vaccinated with 3 μg MERS LNP-mRNA Singlet elicited high titers of MERS binding antibodies with little or no cross reactivity to WT, Delta or SARS spikes (FIGS. 40B-40C, FIG. 43E), suggesting that the MERS LNP-mRNA, when used alone at high dose, has sufficient immunogenicity. Combined with an equal mass of MERS LNP-mRNA, Delta or SARS LNP-mRNA, the two MERS Duplexes also exhibited strong binding antibody titers against cognate antigens (Delta and SARS spike respectively, plus MERS spike) (FIGS. 40B-40C, FIG. 43E).

Meanwhile they also showed cross-reactive response to counterpart spike (For example, MERS+SARS2 Delta against SARS spike, or vice versa) at a lower level than the cognate response (FIGS. 40B-40C, FIG. 43E). Similar with the Triplex experiment, the ELISA ECD activity highly correlated with RBD (FIG. 40D).

Subsequent studies again tested the neutralization activities using the same pseudovirus assays. Mice vaccinated with 3 μg MERS LNP-mRNA Singlet elicited potent MERS neutralizing antibody response with little or no cross reactivity to WT, Delta or SARS spikes (FIGS. 40E-40F), suggesting that the antibodies induced by MERS vaccination alone does not cross-react with SARS or SARS2. Both MERS Duplexes also exhibited strong neutralization activities against MERS, as well as cognate species (MERS+SARS2-Delta for SARS2; and MERS+SARS for SARS) (FIGS. 40E-40F). Interestingly, although the MERS +SARS LNP-mRNA elicited binding antibodies that cross reacted with both WT and Delta spike antigens (FIG. 40B), the induced cross-reactive antibodies only significantly neutralized the WT but not Delta pseudovirus (FIG. 40F). Consistent with prior Triplex-CoV experiment, the ELISA ECD panel correlated well with RBD panel results (FIG. 40G) and tend to have higher titers than RBD panel. Overall, such neutralization activities also significantly correlated with antibody binding against ECD (ELISA) for all groups or all mice, among the spike antigens and pseudoviruses tested (FIG. 40G).

### Single Cell Immune Repertoire Mapping of Multiplexed LNP-mRNA Vaccinated Animals

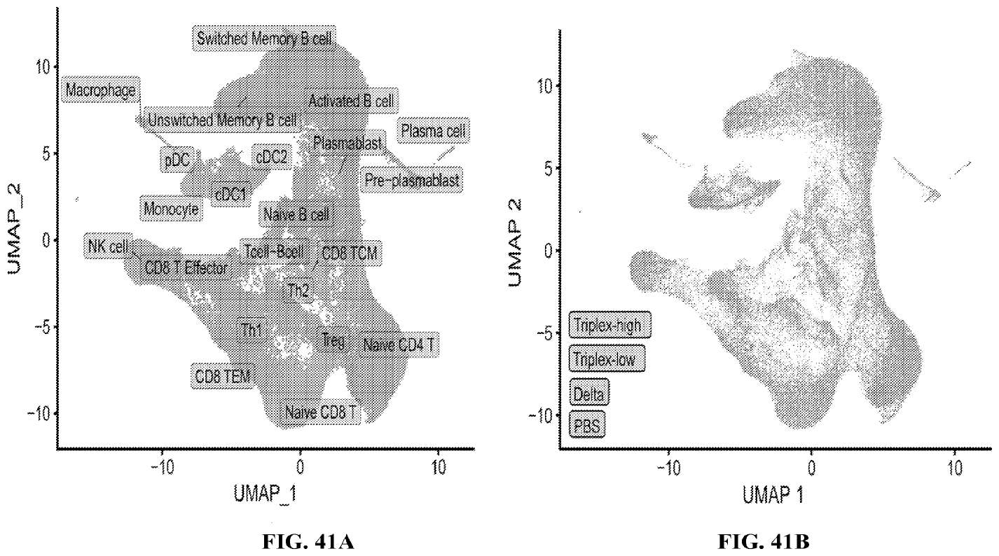

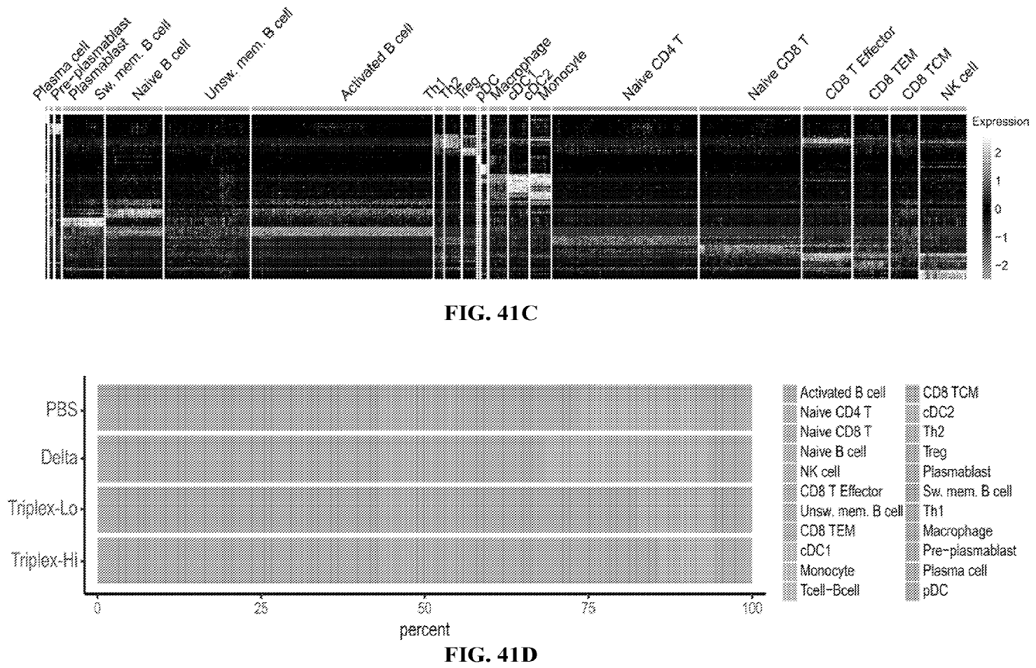

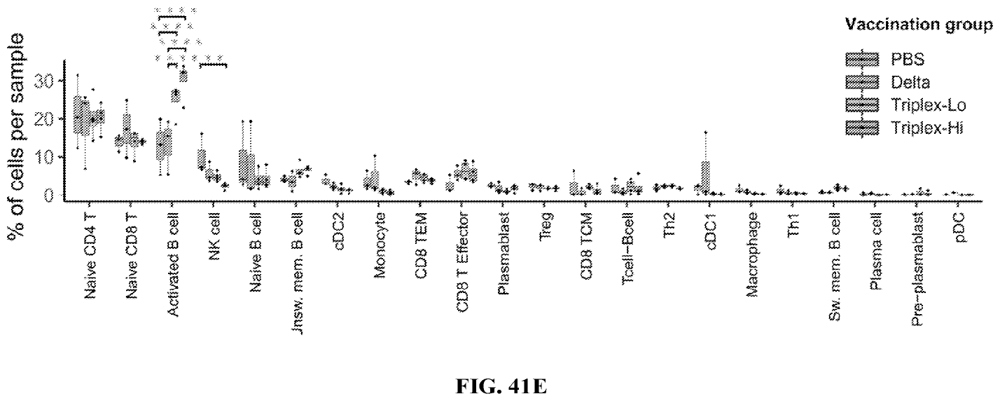

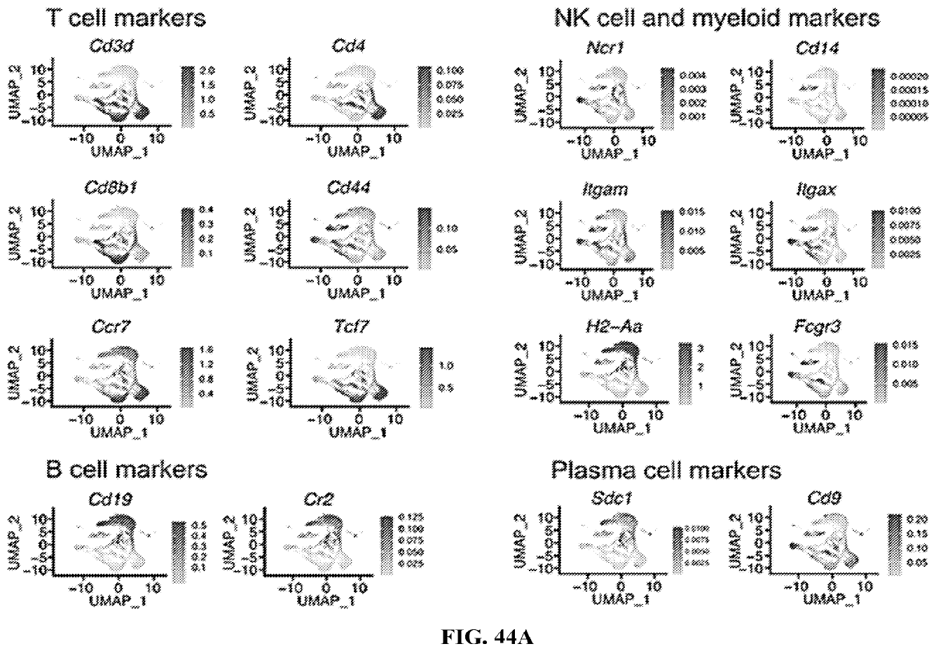

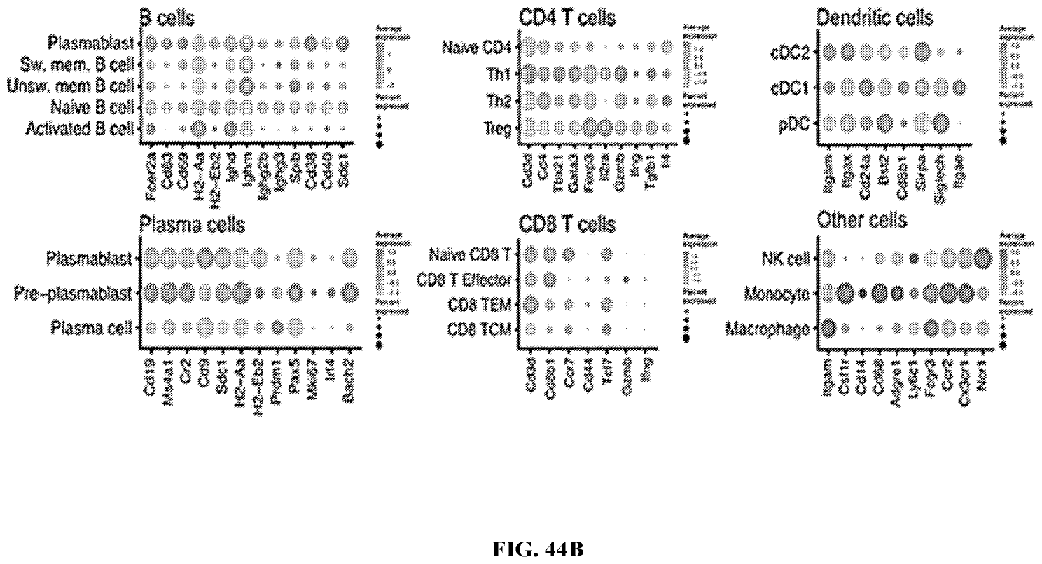

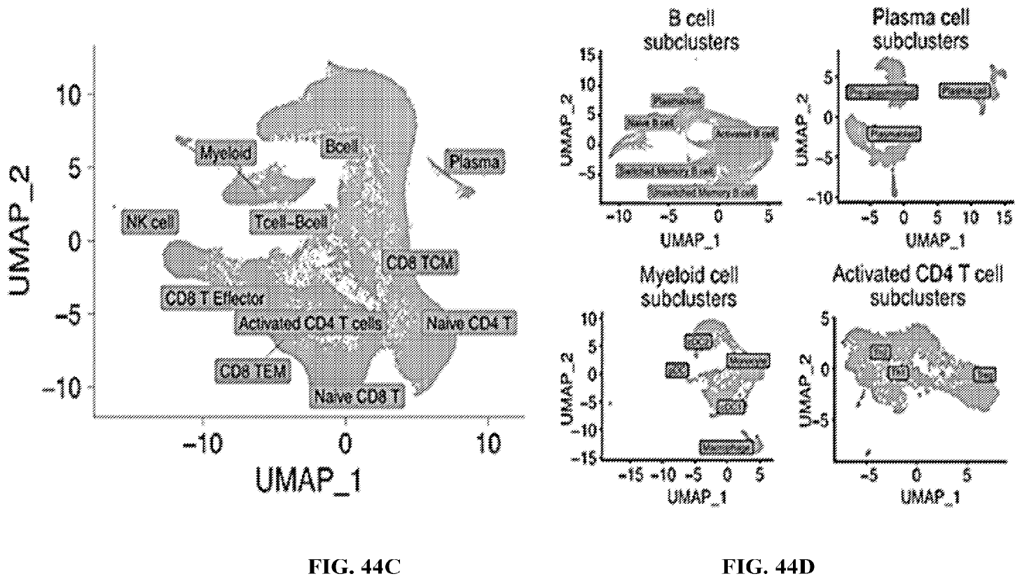

In order to gain insights on the global composition and transcriptional landscape of the immune cells, single cell RNA-seq (scRNA-seq, scGEX) was performed for immune-transcriptomics on the PBMC samples of Delta and Triplex LNP-mRNA vaccinated animals. The use of PBMC samples allows to collect immune cell samples without sacrificing mice so that it is possible to monitor live animals' antibody response over time. As visualized in an overall Uniform Manifold Approximation and Projection (UMAP), from a total of 12 animals from 4 vaccination groups (Delta 1 μg, Triplex-CoV 1 μg and 3 μg dose groups), plus a placebo control group (PBS), we sequenced the transcriptomes of a total of 91,526 single cells, which were visualized in reduced dimensional space by UMAP and clustered to identify cell population structure (FIGS. 41A-B). Using the expression of a set of canonical cell type specific markers, we identified 21 cell clusters as distinct immune cell populations (FIG. 41A; FIGS. 44A-44D). In this dataset, the identified cell clusters include various subsets of B lymphocytes (naïve B cell, activated B cell, unswitched memory B cell, switched memory B cell, pre-plasmablast, plasmablast and plasma cell); T lymphocytes of various subsets (naïve CD8 T cell, CD8 T effector, CD8 central memory T cell (Tcm), CD8 effector memory T cell (Tem), naïve CD4 T cell, Th1 type CD4 T cell, Th2 type CD4 T cell, regulatory T cell (Treg)); dendritic cells (DCs) of various subsets (pDC, cDC1, cDC2); as well as other immune cells (natural killer (NK) cell, macrophage and monocytes) (FIG. 41A). These immune cell populations have distinct gene expression signatures that clearly defined each population against others (FIG. 41C), for example, distinct expression (in terms of both mean expression level and percentage in cluster) of Cd19+H2-Aa+Ighd+Fcer2a+Cd27−defines activated B cells; Cd9+Sdc1+Cd19−Pax5−lo defines plasma cells (FIG. 41C; FIGS. 44A-B). Similarly, for T cell subset examples, Cd3d+Cd4+Tbx21+Gzmb+marks Th1 CD4 T cells; Cd3d+Cd4+Foxp3+Il2ra+marks Tregs; Cd3d+Cd8b1+Ccr7+Cd44−Tcf7+defines naïve CD8 T cells; Cd3d+Cd8b1+Tcf7−Cd44+Ccr7− defines CD8 effector T cells; Itgam+Itgax+Cd24a−Sirpa+defines cDC2 cells; Itgam−Itgax+Bst2+Siglech+defines pDC; Ncr1 defines NK cells; and Itgam+Csflr+Cd14+ defines monocytes (FIG. 41C; FIGS. 44A-B).

Studies then quantified the fractions of each cell type in each sample, to reveal a full picture of immune cell compositions in all vaccination groups profiled (FIGS. 41D-E). With these quantitative fractions, we then compared the systemic immune cell compositions between placebo and vaccinated animals (FIG. 41D). While most of the clusters did not show significant difference in a gross cell population level, three populations (activated B cells, unswitched memory B cells and NK cells) showed significant differences between groups (FIGS. 41D-41E). Interestingly, Triplex-CoV/MixCoV at both high and low doses of vaccination showed significantly increased level of activated B cell populations compared to both PBS and to Delta groups (FIGS. 41D-41E). Both activated and memory B cell populations have been previously implicated for their important roles in SARS-CoV-2 immunity.

### Transcriptomic Signatures of B and T Cell Populations of Triplex LNP-mRNA Vaccinated Animals

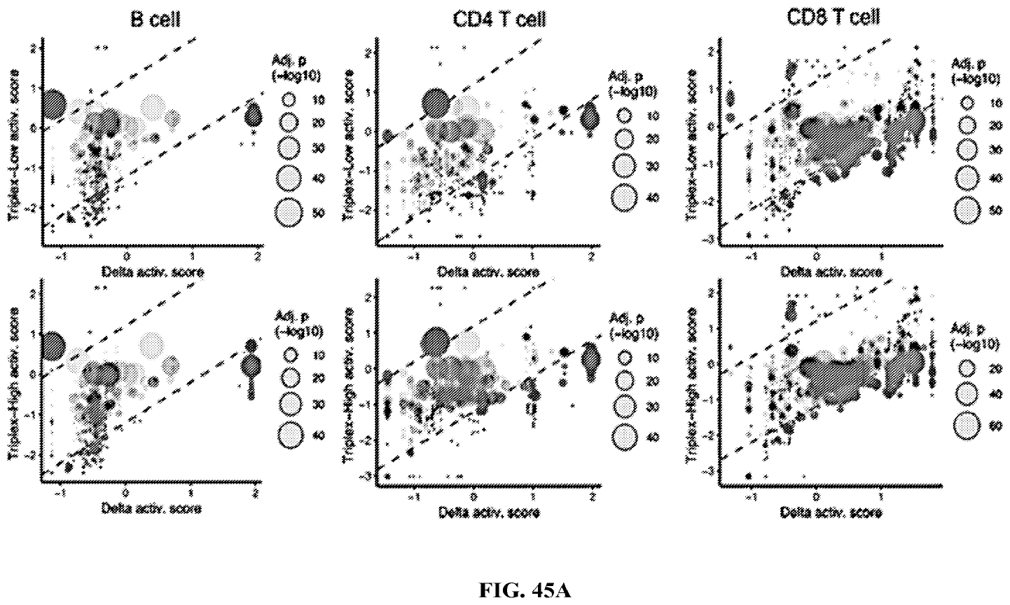

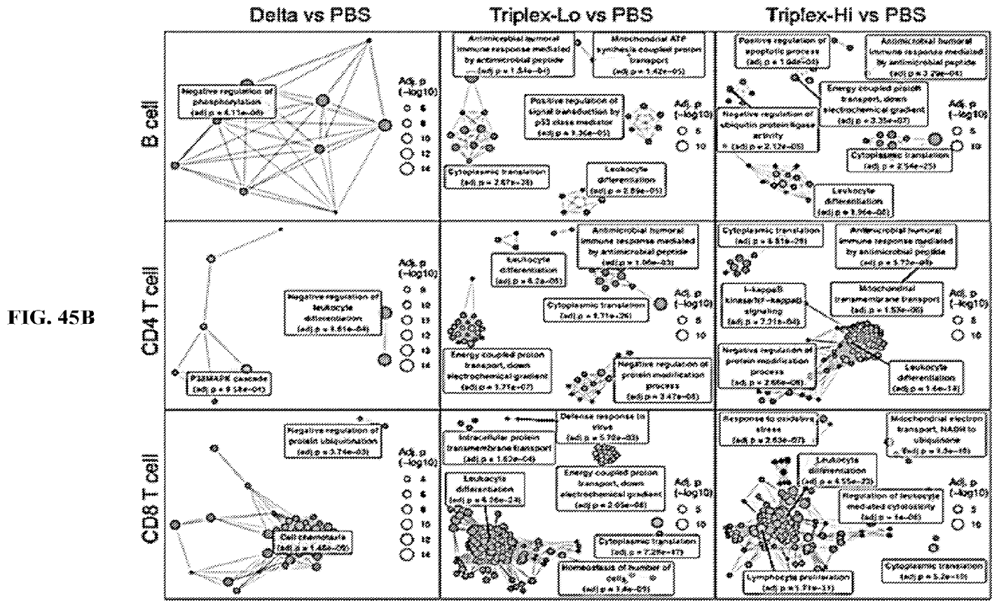

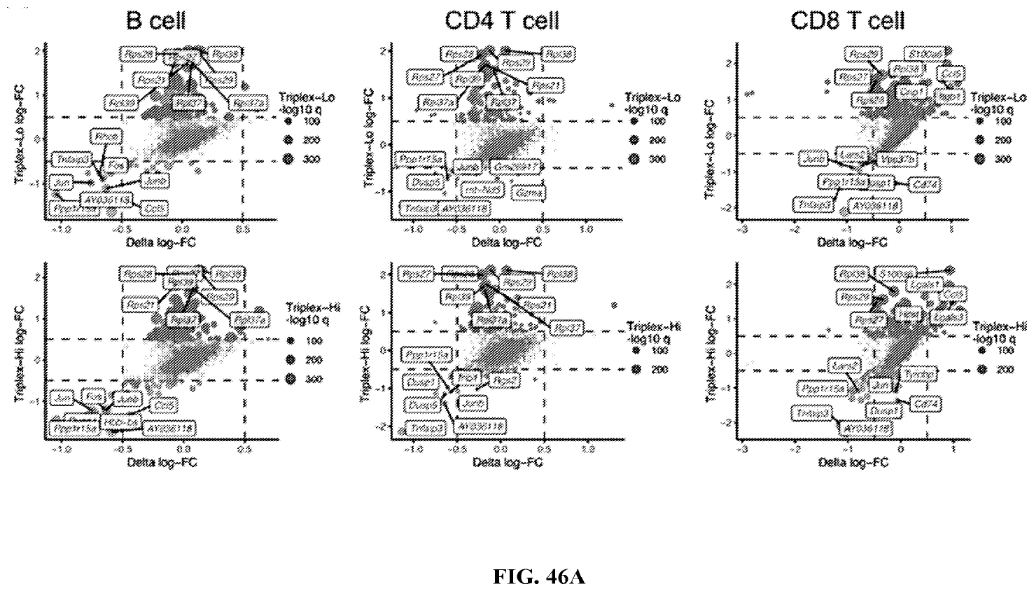

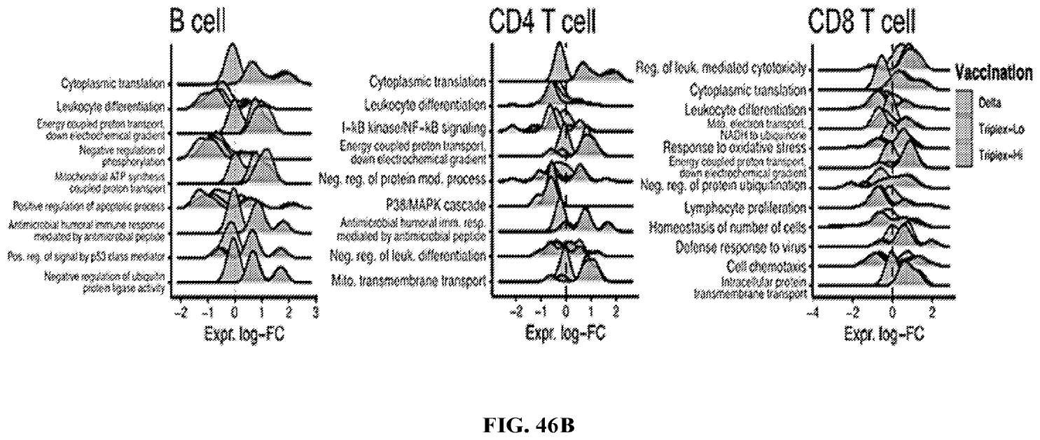

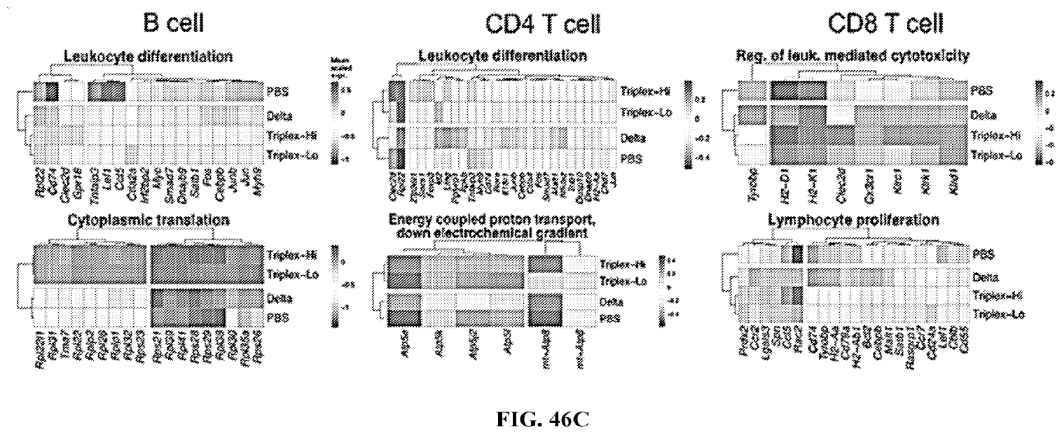

To examine the transcriptomic changes in the immune cell sub-populations upon vaccination, subsequent studies then performed differential expression (DE) analysis in the matched sub-populations between PBS and the several LNP-mRNA groups. We focused on the major adaptive immune cell populations, i.e. the pan activated B cell population (including all identified activated B cell subsets, merged as “B cell”), pan activated CD4 T cell population (all identified activated CD4 T cell subsets, “CD4 T cell”) and pan activated CD8 T cell population (all identified activated CD8 T cell subsets, “CD8 T cell”). Vaccination caused substantial transcriptome changes in the host animals' B cells, CD4 T cells and CD8 T cells, as evidenced by the differential gene expression from vaccinated (Delta, Triplex-CoV/MixCoV low and high dose groups) as compared to the PBS group (FIGS. 45-46). To gain a broad, unbiased view of these transcriptomic changes, we performed a series of gene set and pathway analyses. These analyses revealed a number of altered pathways in the vaccinated animals B cells, CD4 T cells and CD8 T cells as compared to the PBS group (FIG. 45A). Because the altered pathways are diverse, we also performed clustering analysis to uncover the key signal by grouping them into “supra-pathways” where multiple gene sets of similar function were altered. This network analysis of enriched pathways of differentially expressed genes highlighted the most significantly enriched member pathways (as meta-pathway), for the main adaptive immune cell types (B and T cells), for the three vaccination groups (FIG. 45B).

In order to further distinguish the directions, the Ridge density plots were also created, showing the expression log fold change meta-pathway genes between different vaccination groups in different cell types (FIG. 46B). Consistent with the prior observations, the differentially expressed pathways in B cells include leukocyte/lymphocyte mediated immunity in all three vaccination groups compared to PBS (FIG. 45B; FIG. 46B). A top enriched pathway of the differentially expressed genes in B cells is B cell activation, where all three vaccines induced a higher expression of these genes (FIGS. 46B-46C). In CD4 and CD8 T cells, common gene sets are observed, including immune system processes, immune cell differentiation, and T cell activation, consistent with the expected induction from vaccination (FIG. 45B; FIGS. 46B-46C). Interestingly, in T cells, in the differentially expressed genes in all three vaccines, besides regulation of T cell activation, leukocyte proliferation, leukocyte differentiation, defense response to virus and immune responses; basic fundamental pathways are also enriched, especially those involved in core cellular and metabolic functions such as apoptosis, translation, ubiquitin ligase activity, oxidative phosphorylation, mitochondria electron transport, respiratory chain activities (FIGS. 46B-C; FIG. 45B), consistent with the expectation that T cells are metabolically active upon vaccination. The Triplex vaccination induced strong B cell activation pathway clusters in B cells, as well as immune cell differentiation and metabolic activity gene sets in T cells (FIG. 45B; FIGS. 46B-C). These data reveal the broad gene expression signatures at the pathway and cluster levels, across the main adaptive immune cells (B and T cells), for the three vaccination groups studied. The transcriptomic signatures are largely coherent with the literature that these pathways are important for immunity against coronavirus infection and host defense, as well as vaccine-induced immune responses. These data revealed meta-pathway level gene expression changes in the B and T cells' transcriptomes of the animals receiving multiplexed vaccination.

Direct Comparison of Sequential Vs. Simultaneous Vaccination Schedules for LNP-mRNA Vaccination Against Three Species.

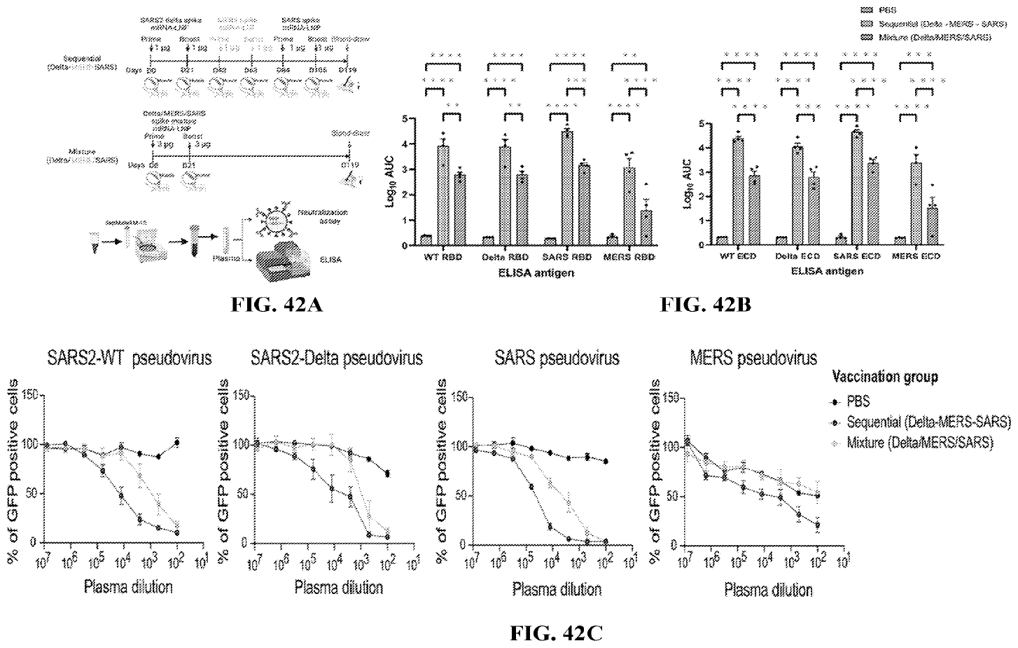

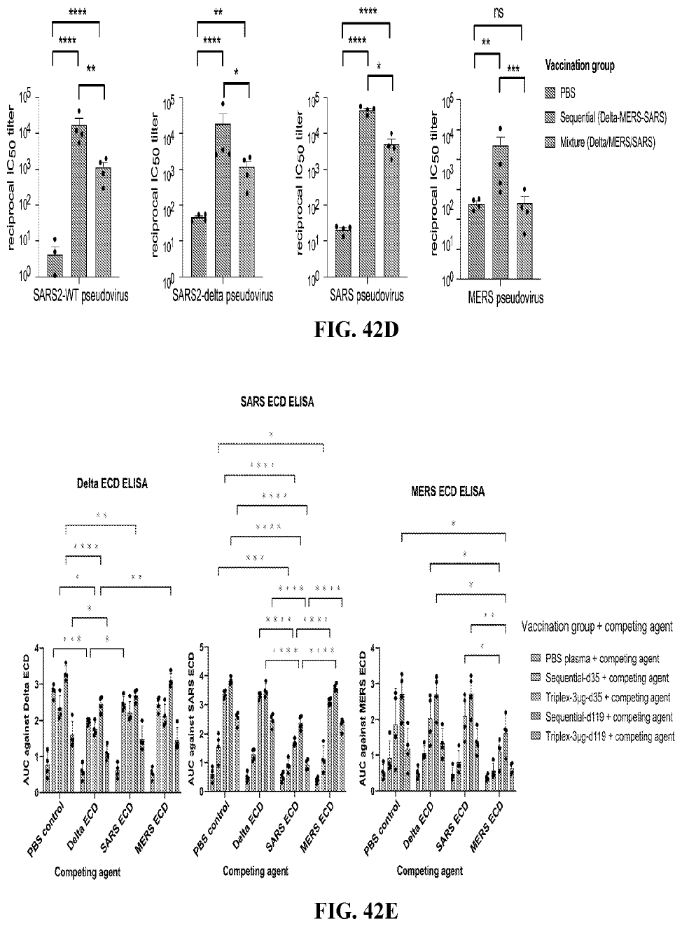

As observed above, Triplex LNP-mRNA vaccination is associated with reduction of antibody responses (FIGS. 38-40), we hypothesized that splitting such vaccination into separate doses may be a strategy to mitigate this loss of effectiveness. It was therefore sought to perform a sequential vaccination schedule and test it in parallel with simultaneous vaccination with mRNAs in mixture (FIG. 42A). In the Sequential vaccination schedule, vaccinations of SARS-CoV-2 Delta, MERS-CoV, and SARS-CoV were given in sequence separated by 3 weeks, each with 1 μg LNP-mRNA prime and 1 μg LNP-mRNA boost 3 weeks apart. In the Mixture vaccination schedule, vaccinations of SARS-CoV-2 Delta, MERS-CoV, and SARS-CoV were given simultaneously, each at 1 μg LNP-mRNA (3 μg total) for both prime and boost. To generate comparable data, we started the first dose at the same day (day 0), and harvested the blood sample at the same day (day 119, i.e. 4 months, post first dose), for both sequential and mixture schedules (FIG. 42A).

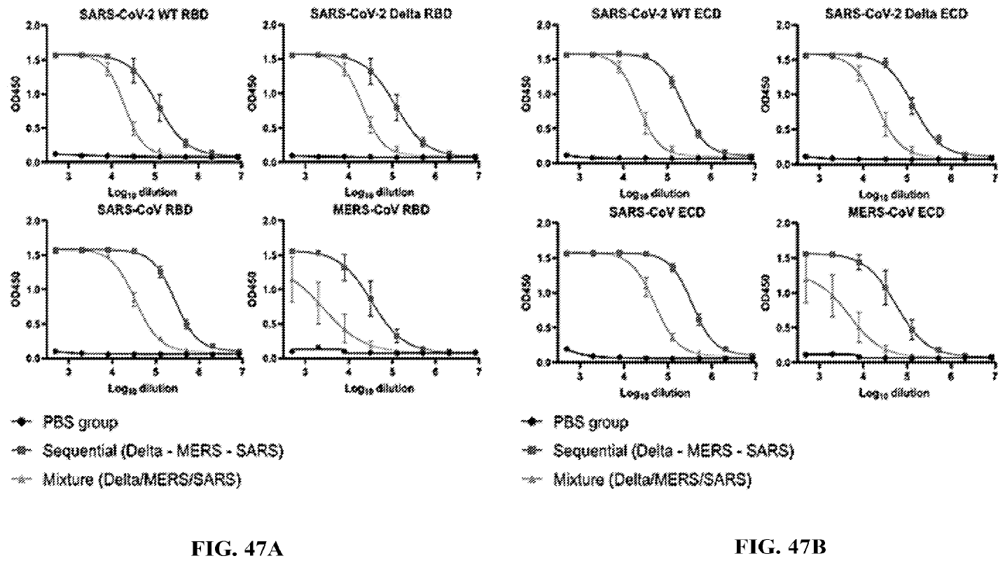

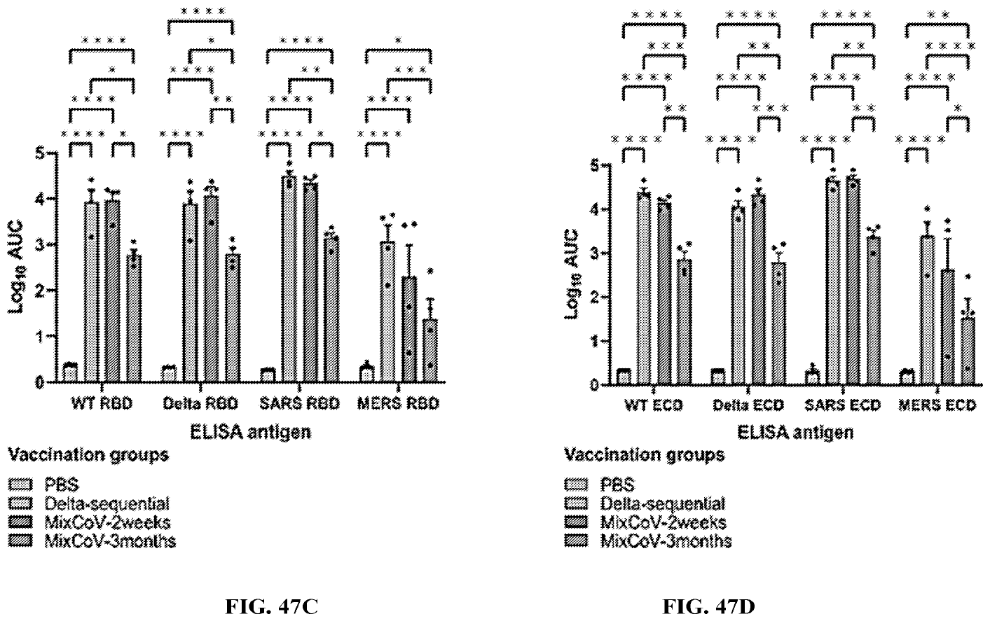

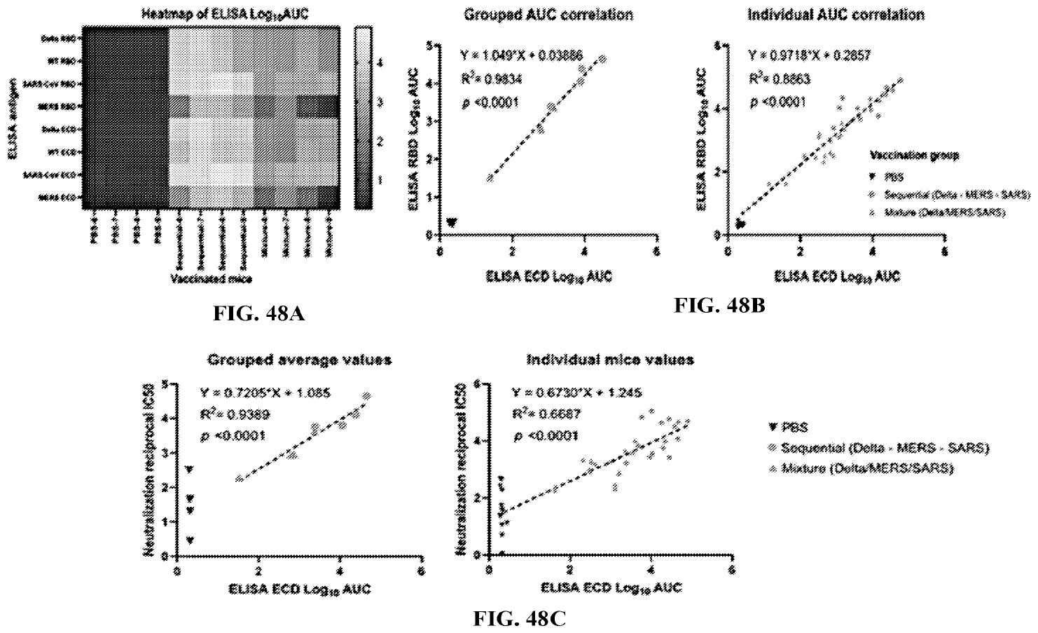

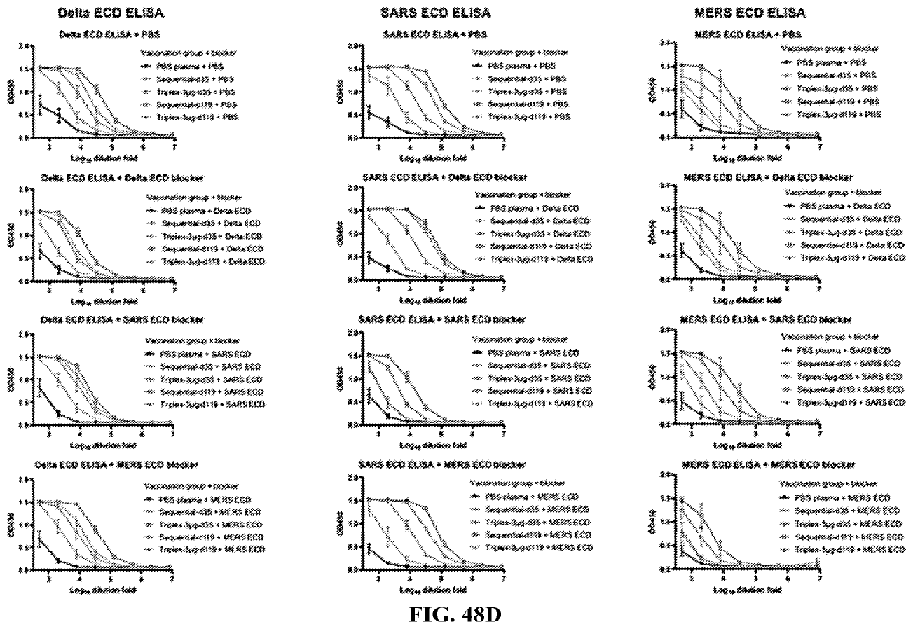

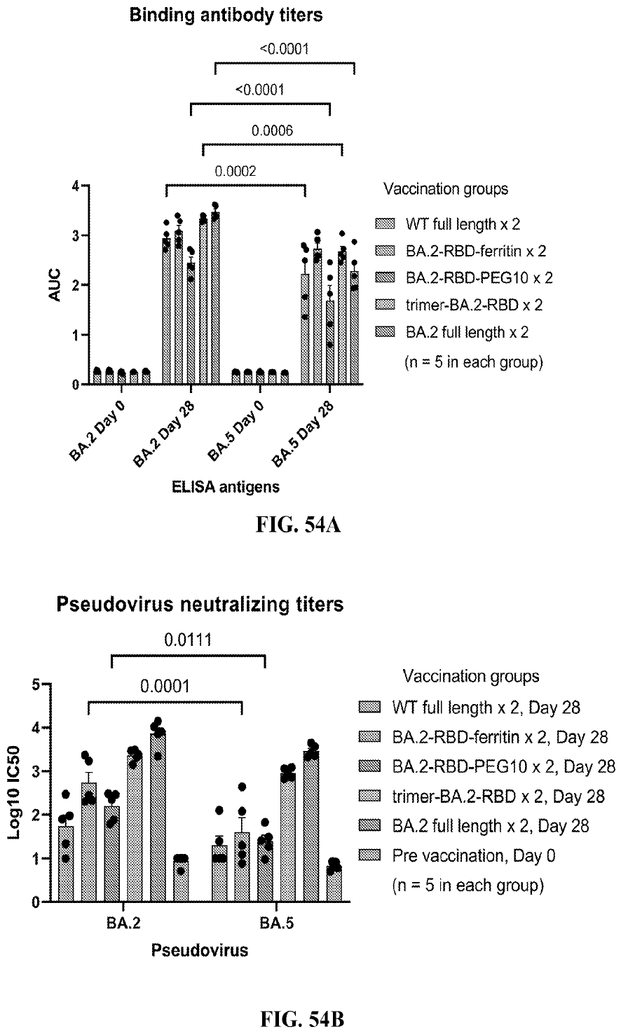

Antibody titers were measured from plasma samples of both Sequential and Mixture LNP-mRNA vaccinated animals (FIG. 42B; FIGS. 47-48). While all vaccinated animals showed certain antibody responses across all antigens tested (SARS2 WT/WA1, SARS2 Delta, SARS, MERS; both ECD and RBD), Sequential vaccination group showed significantly higher antibody responses than Mixture vaccination group across all conditions, i.e. across all antigens from these three species (FIG. 42). Similar with the results above, the ELISA ECD activity highly correlated with that of RBD (FIG. 54B). We tested the neutralization activities using the same pseudovirus assays (FIGS. 42C-42D). Again, mice in the Sequential vaccination schedule showed significantly higher neutralization activities than those in the Mixture vaccination group, and across all three species (FIGS. 42C-42D). Noted that similar to the previous experiment (FIG. 39), the MERS neutralization activity was almost completely lost at this time point in the Mixture vaccination group, yet the Sequential vaccination group retained significant activity above background (FIG. 42D). Overall, such neutralization activities significantly correlated with ECD ELISA for all groups or all mice, among the spike antigens and pseudoviruses tested (FIG. 48C). These data suggested that, for LNP-mRNA vaccination against three coronavirus species under the conditions tested, vaccination in sequence can elicit more potent antibody responses than vaccination simultaneously in mixture.

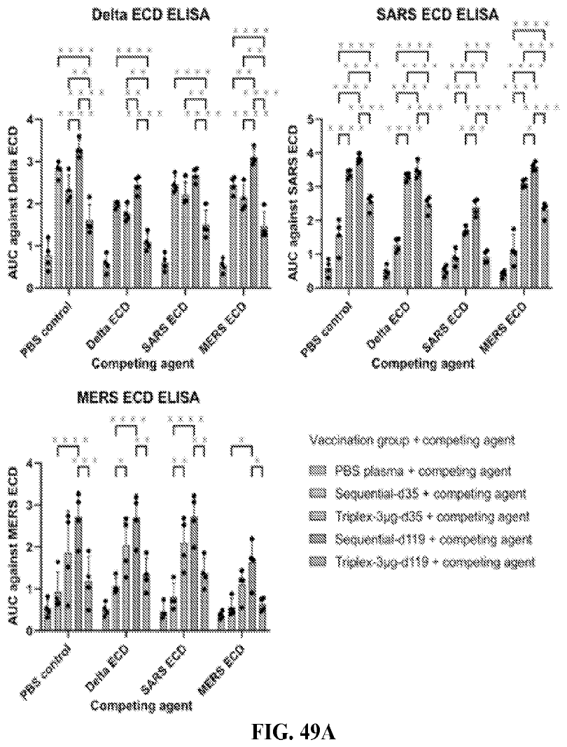

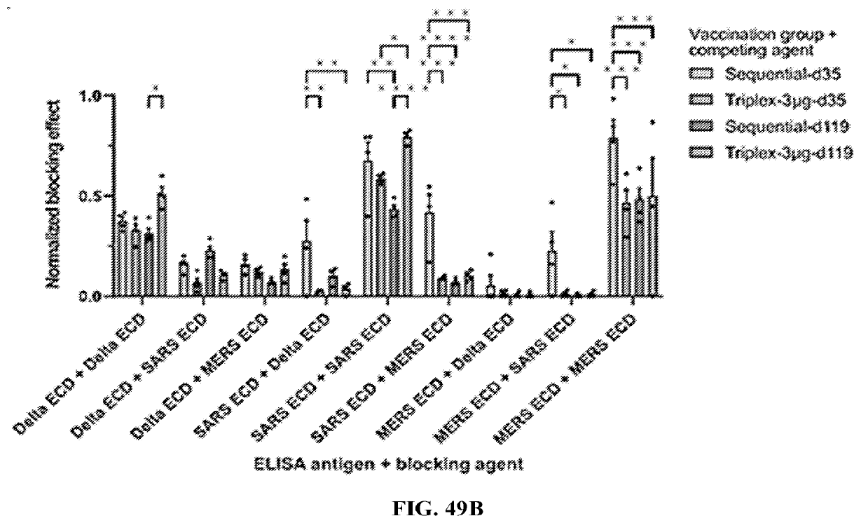

To comprehensively evaluate the cognate and cross-reactive antibody response induced by the Sequential and Mixture LNP-mRNA vaccination, we conducted blocking ELISA where soluble spike antigens or competing agents partially block the plasma antibody response to the homologous or heterologous spike antigen coated on the ELISA plates. The antibody response of Sequential and Triplex samples at matched time points (day 35 in FIG. 38 and day 119 in FIG. 42) in presence and absence of competing agents were directly compared in blocking ELISA. In the absence of competing agents (PBS control), the Triplex's antibody titers against all three spikes (Delta, SARS and MERS ECDs) significantly declined over time (day 35 vs. day 119 in FIG. 42E; FIG. 49). Sequential samples on day 35 before exposure to SARS and MERS antigens only displayed low or moderate activity against MERS and SARS ECDs. At two weeks post final immunization, Sequential samples (day 119) showed a universal trend of higher antibody titers than Triplex (day 35). Under equal mass condition of antigen mRNA in both Sequential and Triplex vaccination, antibody response to SARS was greater than Delta, of which antibody titer was higher than MERS, indicative of distinct immunogenicity of spike antigens from different coronavirus species.

Compared to heterologous blockers, homologous blockers (same spike as ELISA antigen) unequivocally led to greater titer reduction, which ranged from 30% to 70% decrease and represents maximum achievable blocking effect under current conditions (FIG. 42E; FIG. 49). Significant titer reductions by heterologous blockers were associated with cross-species antibodies and observed in Sequential vaccination (day 119) response to Delta ECD by SARS blocker and Sequential/Delta vaccination (day 35) response to SARS ECD by MERS blocker (comparison bracket colored in red in FIG. 42E). Most of heterologous blockers mediated very limited antibody titer reduction, suggesting that cross-coronavirus species antibodies, if exist, only account for a small population of Sequential or Triplex vaccine-induced antibodies. The fact that no heterologous blocker induced significant titer changes in Triplex group suggests that simultaneous exposure to all three coronavirus spike antigens mainly elicits species-specific antibodies, not cross-species antibodies. In most cases, Sequential (day35) or Delta vaccination showed stronger cross reactivity or heterologous blocking effect than other vaccination schemes (FIG. 49), except for Sequential vaccination (day 119) response to Delta ECD by SARS blocker. It is worth noting that despite of the absence of Delta antigen stimulation in Sequential vaccination since day 21, subsequent SARS and MERS antigen immunizations further elevated antibody titer against Delta. The significant blocking effect of SARS ECD on Sequential vaccination (day 119) response to Delta ECD revealed that the Delta titer increase by heterologous boosters was mainly mediated by SARS antigen not MERS antigen. Interestingly, the Sequential vaccination (day 119 vs. day 35) lost strong heterologous blocking effect in SARS and MERS ELISA panels (FIG. 49) suggests that the SARS and MERS antigens predominantly elicited cognate species-specific antibodies, eclipsing the cross-species antibodies observed in Delta vaccination.

### Selected Discussion

Pathogenic coronaviruses have emerged multiple times and infected human populations, several of which (SARS-CoV, MERS-CoV, SARS-CoV-2) have caused severe diseases and fatalities. Several existing less pathogenic coronavirus species (e.g. NL63, 2293, OC43, and HKU1) have been reported to have evolved hundreds to tens of thousands of years ago, and might have evolved to be co-existing with human without causing severe symptoms. Therefore, it is critical to have vaccines against multiple coronavirus species, ideally as pan-coronavirus vaccines, to help fight not only the current pandemic, but also to prevent the re-emergence of the previously existed pathogenic species, as well as constantly evolving and lurking coronavirus diseases as probable future outbreaks. Equally importantly, it is a long-standing need to gain the fundamental understanding of the immune response and the immunological landscape of joint host responses in the context of multiplex coronavirus vaccine.

Various prior efforts led to the development of SARS and MERS vaccine candidates, although at earlier stages of development. The COVID-19 pandemic urged an international effort for rapid development of vaccines against SARS-CoV-2, leading to multiple successful candidates including the highly efficacious mRNA vaccines. However, all these vaccines target a single species and may not offer sufficient protection against other pathogenic species.

To date no study has tested the multiplexing of mRNA vaccines against three major pathogenic coronavirus species (MERS/SARS/SARS2) in triplex setting, nor in sequence. The study of the present disclosure generated a full-length MERS LNP-mRNA vaccine construct, and tested it alone, in combination with SARS-CoV vaccine, SARS-CoV-2 vaccine, and in triplex. The present study directly generated mRNA vaccine candidates and tested in several LNP-mRNA combinations against MERS-CoV, SARS-CoV and SARS-CoV-2, and profiled the immune responses at the single cell level.

The present study reported the antibody responses of triplex and duplex LNP-mRNA vaccines based on MERS spike in combination with SARS and/or SARS2 Delta spikes. The level of cross-reactivity of induced antibodies was in concordance with the sequence identity between vaccine antigen and binding antigen tested in ELISA and pseudovirus assay. The MERS Duplex vaccines, especially when combined with Delta spike, demonstrated significant efficacy against SARS-CoV, SARS-CoV-2 Delta and MERS-CoV. Different from the prior studies, the antiviral spectrum tested herein covers three highly pathogenic coronavirus species in the Betacoronavirus genus, and goes beyond the group 2b coronavirus category (Sarbecoviruses), as it includes MERS in the Merbecovirus subgenus. The data showed that because of low sequence similarity, the vaccine based on Sarbecovirus (SARS and SARS2) provide little or no protection against MERS, the most fatal coronavirus to date with a 35% mortality rate. To broaden vaccine's anti-coronavirus spectrum, the triplex LNP-mRNA vaccine including SARS, SARS2 and MERS was designed and tested. The relatively low level of MERS neutralizing antibody in Triplex vaccine marked a significant challenge of introducing cross-lineage antigens in multiplex vaccination. In order to overcome this hurdle, we kept the species spectrum but reduced the same-lineage antigen number and increase mRNA doses, which gave rise to the Duplex vaccine design. The MERS+SARS2 Delta Duplex showed potent activity against MERS and SARS2 Delta, and to some degree against SARS by cross-reactivity.

To achieve sufficient and broad protection of neutralizing antibodies in multiplex vaccine against these coronavirus species, the relative composition, or the scheme of vaccination, need to be carefully considered in the future. In addition to the multiplexing approach we showed in this study, there are other ways of inducing protective antibodies against SARS2 Delta, SARS and MERS. The production and manufacturing procedures of multiplexed LNP-mRNA formulations, such as mixing, normalization and encapsulation, may benefit from further optimization and testing in the future. Alternatively, the three spike LNP-mRNAs can be given sequentially to avoid negative interactions between spike antigens seen in triplex vaccine. In fact, this is one of the clinical precautions, where individuals are advised to take the COVID-19 mRNA vaccine at least two weeks away from taking other vaccines. Consistent with this notion, our data with direct comparisons in animal vaccination experiments suggested that, giving the mRNA vaccine shots in sequence may benefit from higher antibody titers over a long period of time than giving mRNAs simultaneously in mixture. We directly compared antibody titers 14 days after final dose of sequential vaccination and triplex vaccination (FIG. 47, Delta-sequential vs. MixCoV-2 weeks). MixCoV group antibody titers against SARS1 and SARS2 variants were comparable to Delta-sequential group, while its MERS immunity tends to be lower than sequential vaccination. This is potentially due to competition in antigens, immunodominance, and other reasons. 3 months after the final dose of triplex vaccine, the titers of triplex vaccination declined by ˜10 fold and were significantly lower than those of sequential vaccination, which maintained high antibody titers against all three coronavirus pathogens at day 119 partially due to continuous vaccine boosting over time.

In summary, this study provided LNP-mRNA vaccine constructs designed to target SARS-CoV, SARS-CoV-2 Delta and MERS-CoV, as well as direct in vivo animal testing and single cell immune profiling results of multiplexed combinations as well as comparative vaccination schedules.

### Example 8: Comparison of Antibody Responses to BA.2 and BA.5 by mRNA Vaccines of Full-Length Spikes and RBD Trimer, Ferritin or hPEG10 Nanoparticles

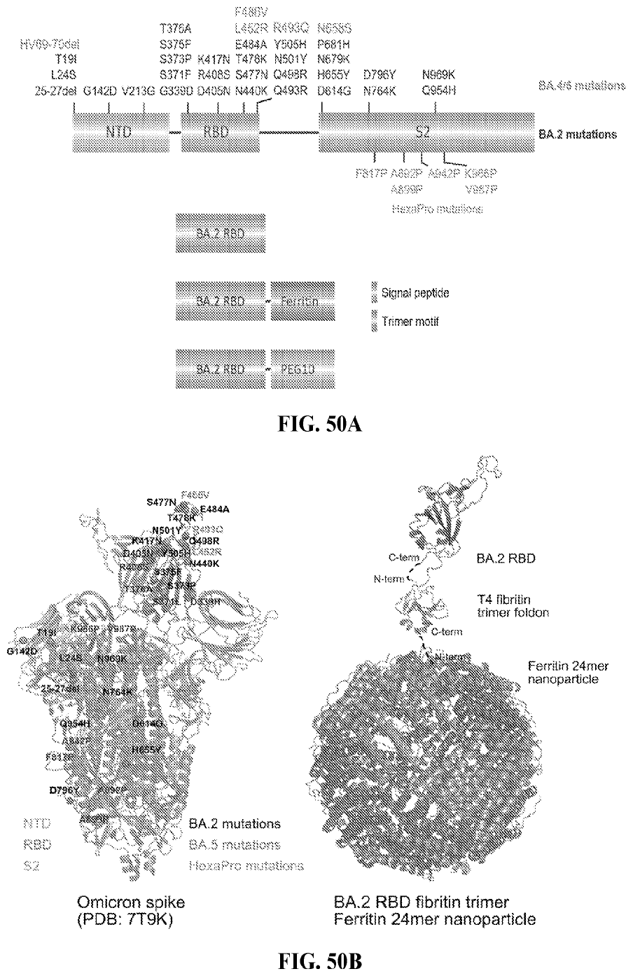

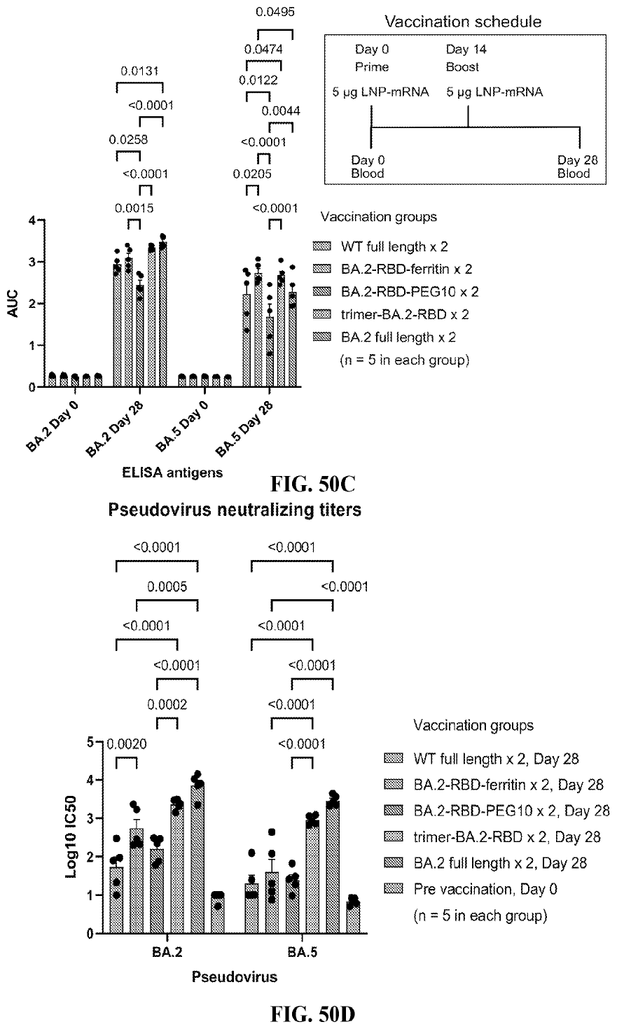

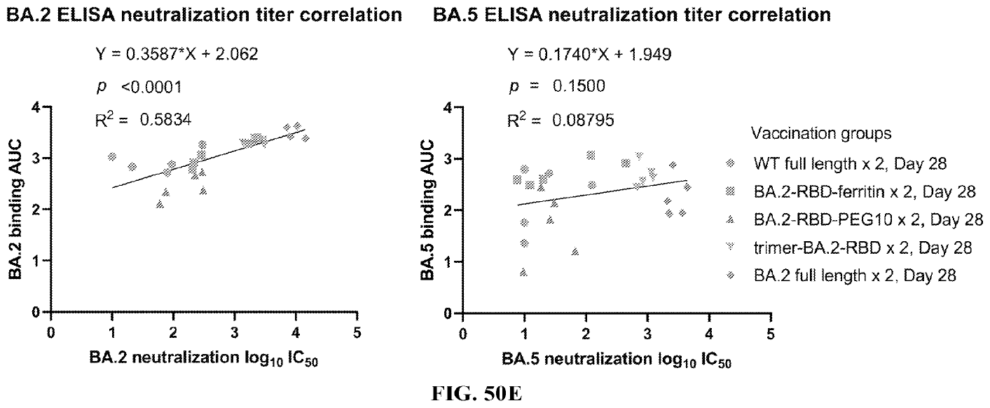

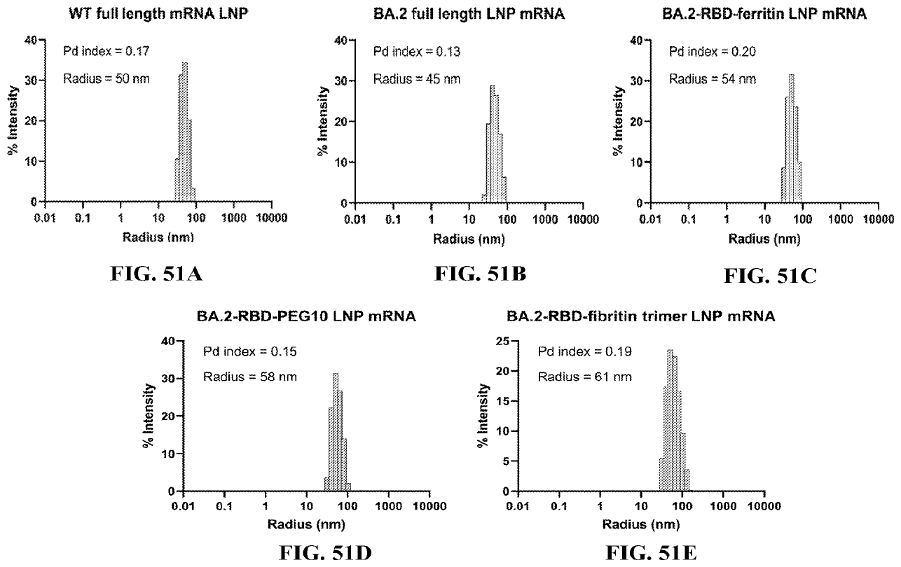

To evaluate the immunogenicity of different types of spike LNP mRNAs, two full length spike LNP mRNAs (WT and BA.2 full lengths) and three BA.2 RBD-based LNP mRNAs were generated (FIG. 50A-50B), which contain N-term tissue plasminogen activator (tPA) signal peptide and C-term T4 fibritin trimer foldon (BA.2-RBD-trimer). The ferritin or human PEG10 (hPEG10) nanoparticle sequence was appended to the C terminus of two RBD-based LNP mRNAs (BA.2-RBD-ferritin/hPEG10). The size distribution of five generated LNPs were characterized by dynamic light scattering, which showed homogenous and monodispersed nanoparticles on the radius histogram (FIG. 51). Mice were immunized with two doses of 5 μg same spike LNP mRNAs on day 0 and day 14 (FIG. 50C). Plasma samples were isolated from blood collected on day 0 and day 28 for evaluating binding and neutralizing antibody response in ELISA and pseudovirus neutralization assay.

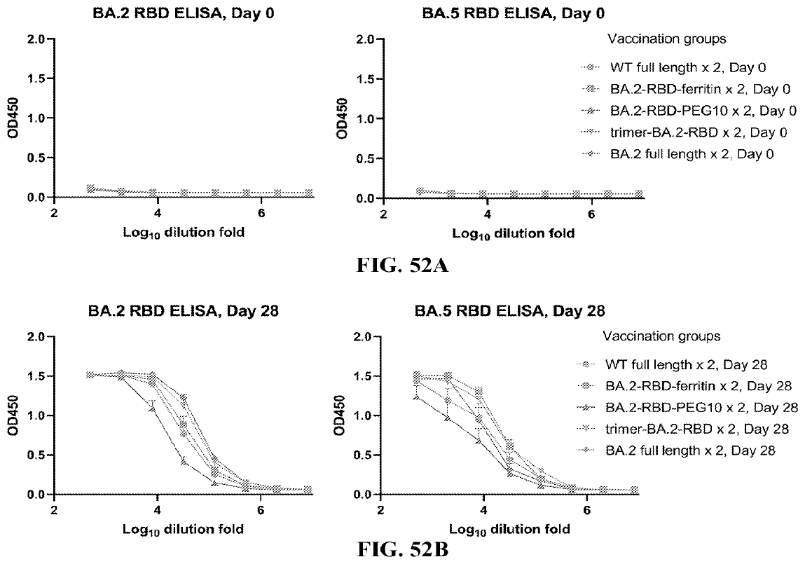

As expected, no spike RBD binding antibody was detected in all pre-vaccination samples collected on day 0 (FIG. 52 and FIG. 50C). Two weeks post boost, all five types of spike LNP-mRNAs elicited significant antibodies binding to BA.2 and BA.5 RBDs. Compared to BA.2 antibodies, lower antibody levels and larger variations were observed in BA.5 binding antibodies, suggestive of BA.5 mediated immune evasion from immunity elicited by WT or BA.2 LNP mRNA. The antibody titers induced by BA.2-RBD-PEG10 LNP mRNA were significantly lower than other four LNP mRNAs. BA.2 binding antibody titer of BA.2 full length LNP mRNA was significantly higher than that of WT LNP mRNA.

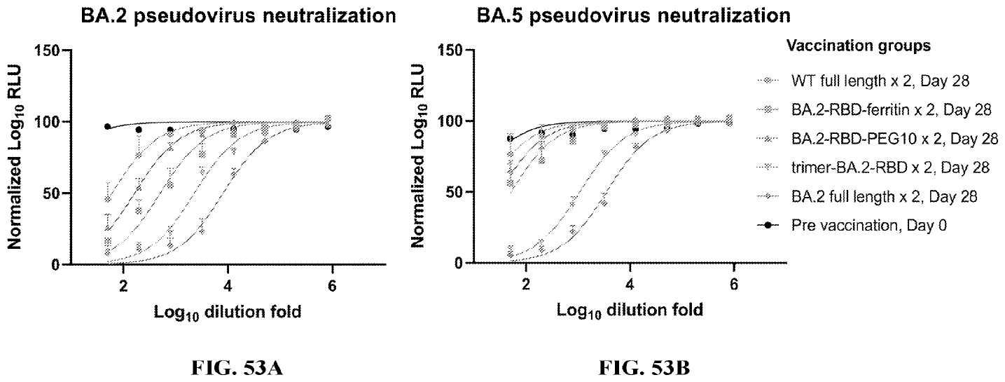

The BA.5 pseudovirus neutralizing titers separated five vaccination groups into two categories with low or high neutralizing activity (FIG. 53 and FIG. 50d). The BA.2 full length and BA.2-RBD-trimer vaccination groups exhibited high neutralizing activity against both BA.2 and BA.5, suggesting that their neutralizing antibodies maintained high cross reactivity with BA.5 subvariant. With much shorter antigen sequence, the BA.2-RBD-trimer induced neutralizing antibody titers comparable to that of BA.2 full length. The WT full length and BA.2-RBD-ferritin/hPEG10 vaccination groups showed limited BA.5 neutralizing activity. Among the five vaccination groups, more evident drops of neutralizing titers from BA.2 to BA.5 datasets were observed in BA.2-RBD-ferritin/hPEG10 vaccination groups (FIG. 54), suggesting that BA.2 RBD presented on ferritin or hPEG10 nanoparticles induced neutralizing antibodies with low BA.5 cross reactivity.

The BA.2 binding and neutralizing antibody titers were well correlated with greater model deviations from the WT full length group, which showed high binding antibodies but low neutralizing titer (FIG. 50E). The BA.5 binding and neutralizing titers were not significantly correlated due to large variations in binding and neutralizing titers in the same group.

pZF72 tPA BA.2 RBD tri-ferritin
(SEQ ID NO: 123)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATGGACGCCATGAAGAGAGGCCTGTGCTGCGTGCTGCTGCTGTGCGGCGCCGTGTTCGTGAG
CCCTAGCAGGGTCCAACCAACAGAGAGCATTGTGAGGTTTCCAAACATCACCAACCTGTGTCCATTTG
acGAGGTGTTCAAcGCCACCAGGTTTGCCTCTGTCTATGCCTGGAACAGGAAGAGGATTAGCAACTGT
GTGGCTGACTACTCTGTGCTCTACAACTTCGCCcCCTTCttCGCCTTCAAGTGTTATGGAGTGAGCCC
AACCAAACTGAATGACCTGTGTTTCACCAATGTCTATGCTGACTCCTTTGTGATTAGGGGAAACGAGG
TGAGCCAGATTGCCCCTGGACAAACAGGCAAcATTGCTGACTACAACTACAAACTGCCTGATGACTTC
ACAGGCTGTGTGATTGCCTGGAACAGCAACAAgCTGGACAGCAAGGTGGGCGGCAACTACAACTACCT
CTACAGACTGTTCAGGAAGAGCAACCTGAAACCATTTGAGAGGGACATCAGCACAGAGATTTACCAGG
CTGGCAaCAagCCATGTAATGGAGTGGccGGCTTCAACTGTTACTTTCCACTCCggTCCTATGGCTTC
CggCCAACCtacGGAGTGGGCcACCAACCATACAGGGTGGTGGTGCTGTCCTTTGAACTGCTCCATGC
CCCTGCCACAGTGTGTGGACCAAAGAAGAGCACCAACCTGGTGAAGAACAAGTGTGTGAACTTCGGCA
GCGGCGGCTACATCCCCGAGGCCCCTAGAGACGGCCAAGCCTACGTGAGAAAGGACGGCGAATGGGTA
CTCCTGAGCACCTTCCTGGGTGGCGGAGGCAGCGGCGGGGGAGGCAGCCTGTCCAAGGACATCATCAA
GCTGCTGAACGAGCAAGTGAACAAGGAGATGAACAGCAGCAACCTGTACATGAGCATGAGCAGCTGGT
GCTACACCCACAGCCTGGACGGCGCCGGCCTGTTCCTGTTCGACCACGCCGCCGAGGAGTACGAGCAC
GCCAAGAAGCTGATCATCTTCCTGAACGAGAACAACGTGCCCGTGCAGCTGACAAGCATCAGCGCCCC
CGAGCACAAGTTCGAGGGCCTGACACAGATCTTTCAGAAGGCCTACGAGCACGAGCAGCACATCAGCG
AGAGCATCAACAACATCGTGGACCACGCCATCAAGAGCAAGGACCACGCCACCTTCAACTTCCTGCAG
TGGTACGTGGCCGAGCAGCACGAGGAAGAGGTGCTGTTCAAGGACATCCTGGACAAGATCGAGCTGAT
CGGCAACGAGAACCACGGCCTGTACCTGGCCGATCAGTACGTGAAGGGCATCGCCAAGAGCAGAAAGA
GCTGATAATAGCTCGAGCTGGTACTGCATGCACGCAATGCTAGCTGCCCCTTTCCCGTCCTGGGTACC
CCGAGTCTCCCCCGACCTCGGGTCCCAGGTATGCTCCCACCTCCACCTGCCCCACTCACCACCTCTGC
TAGTTCCAGACACCTCCCAAGCACGCAGCAATGCAGCTCAAAACGCTTAGCCTAGCCACACCCCCACG
GGAAACAGCAGTGATTAACCTTTAGCAATAAACGAAAGTTTAACTAAGCTATACTAACCCCAGGGTTG
GTCAATTTCGTGCCAGCCACACCCTGGAGCTAGCaaaccgctgatcagcctcgactgtgccttctagt
tgccagccatctgttgtttgcccctcccccgtgccttccttgaccctggaaggtgccactcccactgt
cctttcctaataaaatgaggaaattgcatcgcattgtctgagtaggtgtcattctattctggggggtg
gggtggggcaggacagcaagggggaggattgggaagacaatagcaggcatgctggggatgcggtgggc
tctatggcttctgaggcggaaagaaccagctggggctctagggggtatccccacgcgccctgtagcgg
cgcattaagcgcggcgggtgtggtggttacgcgcagcgtgaccgctacacttgccagcgccctagcgc
ccgctcctttcgctttcttcccttcctttctcgccacgttcgccggctttccccgtcaagctctaaat
cgggggctccctttagggttccgatttagtgctttacggcacctcgaccccaaaaaacttgattaggg
tgatggttcacgtagtgggccatcgccctgatagacggtttttcgccctttgacgttggagtccacgt
tctttaatagtggactcttgttccaaactggaacaacactcaaccctatctcggtctattcttttgat
ttataagggattttgccgatttcggcctattggttaaaaaatgagctgatttaacaaaaatttaacgc
gaattaattctgtggaatgtgtgtcagttagggtgtggaaagtccccaggctccccagcaggcagaag
tatgcaaagcatgcatctcaattagtcagcaaccaggtgtggaaagtccccaggctccccagcaggca
gaagtatgcaaagcatgcatctcaattagtcagcaaccatagtcccgcccctaactccgcccatcccg
cccctaactccgcccagttccgcccattctccgccccatggctgactaattttttttatttatgcaga
ggccgaggccgcctctgcctctgagctattccagaagtagtgaggaggcttttttggaggcctaggct
tttgcaaaaagctcccgggagcttgtatatccattttcggatctgatcaagagacaggatgaggatcg
tttcgcatgattgaacaagatggattgcacgcaggttctccggccgcttgggtggagaggctattcgg
ctatgactgggcacaacagacaatcggctgctctgatgccgccgtgttccggctgtcagcgcaggggc
gcccggttctttttgtcaagaccgacctgtccggtgccctgaatgaactgcaggacgaggcagcgcgg
ctatcgtggctggccacgacgggcgttccttgcgcagctgtgctcgacgttgtcactgaagcgggaag
ggactggctgctattgggcgaagtgccggggcaggatctcctgtcatctcaccttgctcctgccgaga
aagtatccatcatggctgatgcaatgcggcggctgcatacgcttgatccggctacctgcccattcgac
caccaagcgaaacatcgcatcgagcgagcacgtactcggatggaagccggtcttgtcgatcaggatga
tctggacgaagagcatcaggggctcgcgccagccgaactgttcgccaggctcaaggcgcgcatgcccg
acggcgaggatctcgtcgtgacccatggcgatgcctgcttgccgaatatcatggtggaaaatggccgc
ttttctggattcatcgactgtggccggctgggtgtggcggaccgctatcaggacatagcgttggctac
ccgtgatattgctgaagagcttggcggcgaatgggctgaccgcttcctcgtgctttacggtatcgccg
ctcccgattcgcagcgcatcgccttctatcgccttcttgacgagttcttctgagcgggactctggggt
tcgaaatgaccgaccaagcgacgcccaacctgccatcacgagatttcgattccaccgccgccttctat
gaaaggttgggcttcggaatcgttttccgggacgccggctggatgatcctccagcgcggggatctcat
gctggagttcttcgcccaccccaacttgtttattgcagcttataatggttacaaataaagcaatagca
tcacaaatttcacaaataaagcatttttttcactgcattctagttgtggtttgtccaaactcatcaat
gtatcttatcatgtctgtataccgtcgacctctagctagagcttggcgtaatcatggtcatagctgtt
tcctgtgtgaaattgttatccgctcacaattccacacaacatacgagccggaagcataaagtgtaaag
cctggggtgcctaatgagtgagctaactcacattaattgcgttgcgctcactgcccgctttccagtcg
ggaaacctgtcgtgccagctgcattaatgaatcggccaacgcgcggggagaggcggtttgcgtattgg
gcgctcttccgcttcctcgctcactgactcgctgcgctcggtcgttcggctgcggcgagcggtatcag
ctcactcaaaggcggtaatacggttatccacagaatcaggggataacgcaggaaagaacatgtgagca
aaaggccagcaaaaggccaggaaccgtaaaaaggccgcgttgctggcgtttttccataggctccgccc
ccctgacgagcatcacaaaaatcgacgctcaagtcagaggtggcgaaacccgacaggactataaagat
accaggcgtttccccctggaagctccctcgtgcgctctcctgttccgaccctgccgcttaccggatac
ctgtccgcctttctcccttcgggaagcgtggcgctttctcatagctcacgctgtaggtatctcagttc
ggtgtaggtcgttcgctccaagctgggctgtgtgcacgaaccccccgttcagcccgaccgctgcgcct
tatccggtaactatcgtcttgagtccaacccggtaagacacgacttatcgccactggcagcagccact
ggtaacaggattagcagagcgaggtatgtaggcggtgctacagagttcttgaagtggtggcctaacta
cggctacactagaagaacagtatttggtatctgcgctctgctgaagccagttaccttcggaaaaagag
ttggtagctcttgatccggcaaacaaaccaccgctggtagcggtggtttttttgtttgcaagcagcag
attacgcgcagaaaaaaaggatctcaagaagatcctttgatcttttctacggggtctgacgctcagtg
gaacgaaaactcacgttaagggattttggtcatgagattatcaaaaaggatcttcacctagatccttt
taaattaaaaatgaagttttaaatcaatctaaagtatatatgagtaaacttggtctgacagttaccaa
tgcttaatcagtgaggcacctatctcagcgatctgtctatttcgttcatccatagttgcctgactccc
cgtcgtgtagataactacgatacgggagggcttaccatctggccccagtgctgcaatgataccgcgag
acccacgctcaccggctccagatttatcagcaataaaccagccagccggaagggccgagcgcagaagt
ggtcctgcaactttatccgcctccatccagtctattaattgttgccgggaagctagagtaagtagttc
gccagttaatagtttgcgcaacgttgttgccattgctacaggcatcgtggtgtcacgctcgtcgtttg
gtatggcttcattcagctccggttcccaacgatcaaggcgagttacatgatcccccatgttgtgcaaa
aaagcggttagctccttcggtcctccgatcgttgtcagaagtaagttggccgcagtgttatcactcat
ggttatggcagcactgcataattctcttactgtcatgccatccgtaagatgcttttctgtgactggtg
agtactcaaccaagtcattctgagaatagtgtatgcggcgaccgagttgctcttgcccggcgtcaata
cgggataataccgcgccacatagcagaactttaaaagtgctcatcattggaaaacgttcttcggggcg
aaaactctcaaggatcttaccgctgttgagatccagttcgatgtaacccactcgtgcacccaactgat
cttcagcatcttttactttcaccagcgtttctgggtgagcaaaaacaggaaggcaaaatgccgcaaaa
aagggaataagggcgacacggaaatgttgaatactcatactcttcctttttcaatattattgaagcat
ttatcagggttattgtctcatgagcggatacatatttgaatgtatttagaaaaataaacaaatagggg
ttccgcgcacatttccccgaaaagtgccacctgacgtc
pZF73v4_tPA_BA2_RBD_tri_hPEG10_mRNA
(SEQ ID NO: 124)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATGGACGCCATGAAGAGAGGCCTGTGCTGCGTGCTGCTGCTGTGCGGCGCCGTGTTCGTGAG
CCCTAGCAGGGTCCAACCAACAGAGAGCATTGTGAGGTTTCCAAACATCACCAACCTGTGTCCATTTG
acGAGGTGTTCAAcGCCACCAGGTTTGCCTCTGTCTATGCCTGGAACAGGAAGAGGATTAGCAACTGT
GTGGCTGACTACTCTGTGCTCTACAACTTCGCCcCCTTCttCGCCTTCAAGTGTTATGGAGTGAGCCC
AACCAAACTGAATGACCTGTGTTTCACCAATGTCTATGCTGACTCCTTTGTGATTAGGGGAAACGAGG
TGAGCCAGATTGCCCCTGGACAAACAGGCAAcATTGCTGACTACAACTACAAACTGCCTGATGACTTC
ACAGGCTGTGTGATTGCCTGGAACAGCAACAAgCTGGACAGCAAGGTGGGCGGCAACTACAACTACCT
CTACAGACTGTTCAGGAAGAGCAACCTGAAACCATTTGAGAGGGACATCAGCACAGAGATTTACCAGG
CTGGCAaCAagCCATGTAATGGAGTGGccGGCTTCAACTGTTACTTTCCACTCCggTCCTATGGCTTC
CggCCAACCtacGGAGTGGGCcACCAACCATACAGGGTGGTGGTGCTGTCCTTTGAACTGCTCCATGC
CCCTGCCACAGTGTGTGGACCAAAGAAGAGCACCAACCTGGTGAAGAACAAGTGTGTGAACTTCGGCT
CAGGTGGTTACATACCAGAGGCACCCAGGGACGGTCAGGCCTATGTGAGGAAAGACGGTGAGTGGGTG
TTGCTGTCCACGTTTCTCGGCGGTGGCGGTTCCGGCGGTGGTGGGTCTATGCTTGGCCCTGATTGTCC
GCCACCTCCACCGCCCCCGCCACCCAACAACAATAACAATAATAATAGTAAGCATACAGGTCATAAGA
GTGCGTGCGTTCCTAATATGACAGAGCGGCGCAGGGATGAACTGTCCGAAGAAATTAATAACCTTAGG
GAAAAAGTAATGAAGCAATCCGAGGAAAATAATAATTTGCAATCTCAAGTGCAGAAACTCACGGAAGA
AAATACCACGCTTAGAGAACAGGTGGAGCCGACTCCAGAGGACGAAGATGATGATATTGAGCTCAGGG
GCGCTGCAGCTGCCGCAGCTCCTCCACCTCCGATAGAGGAAGAGTGTCCTGAAGACTTGCCTGAAAAG
TTCGACGGCAACCCTGACATGTTGGCACCTTTTATGGCTCAGTGTCAGATATTTATGGAGAAGAGTAC
CAGGGATTTTTCAGTCGATCGCGTTCGCGTCTGCTTCGTGACGAGCATGATGACCGGTAGAGCTGCTC
GATGGGCTTCAGCTAAGCTTGAACGCTCCCACTATCTTATGCATAATTATCCGGCATTTATGATGGAG
ATGAAGCACGTGTTTGAAGACCCCCAGCGCAGGGAGGTCGCTAAAAGGAAGATAAGGAGACTCAGACA
AGGAATGGGTTCTGTCATTGATTATTCTAACGCTTTCCAGATGATAGCACAGGATCTTGATTGGAATG
AACCCGCTCTTATTGACCAGTACCACGAAGGTCTCTCAGACCATATCCAAGAAGAACTTTCACATCTG
GAGGTCGCTAAGAGCCTGTCTGCTCTCATTGGTCAGTGTATCCATATAGAGCGGCGGCTCGCAAGGGC
GGCTGCGGCCAGAAAACCCCGATCCCCGCCCCGAGCCCTCGTCCTGCCCCATATTGCCTCTCATCATC
AAGTAGATCCTACCGAGCCTGTGGGAGGAGCGCGGATGCGGCTTACCCAAGAGGAATGATAATAGCTC
GAGCTGGTACTGCATGCACGCAATGCTAGCTGCCCCTTTCCCGTCCTGGGTACCCCGAGTCTCCCCCG
ACCTCGGGTCCCAGGTATGCTCCCACCTCCACCTGCCCCACTCACCACCTCTGCTAGTTCCAGACACC
TCCCAAGCACGCAGCAATGCAGCTCAAAACGCTTAGCCTAGCCACACCCCCACGGGAAACAGCAGTGA
TTAACCTTTAGCAATAAACGAAAGTTTAACTAAGCTATACTAACCCCAGGGTTGGTCAATTTCGTGCC
AGCCACACCCTGGAGCTAGCaaaccgctgatcagcctcgactgtgccttctagttgccagccatctgt
tgtttgcccctcccccgtgccttccttgaccctggaaggtgccactcccactgtcctttcctaataaa
atgaggaaattgcatcgcattgtctgagtaggtgtcattctattctggggggtggggtggggcaggac
agcaagggggaggattgggaagacaatagcaggcatgctggggatgcggtgggctctatggcttctga
ggcggaaagaaccagctggggctctagggggtatccccacgcgccctgtagcggcgcattaagcgcgg
cgggtgtggtggttacgcgcagcgtgaccgctacacttgccagcgccctagcgcccgctcctttcgct
ttcttcccttcctttctcgccacgttcgccggctttccccgtcaagctctaaatcgggggctcccttt
agggttccgatttagtgctttacggcacctcgaccccaaaaaacttgattagggtgatggttcacgta
gtgggccatcgccctgatagacggtttttcgccctttgacgttggagtccacgttctttaatagtgga
ctcttgttccaaactggaacaacactcaaccctatctcggtctattcttttgatttataagggatttt
gccgatttcggcctattggttaaaaaatgagctgatttaacaaaaatttaacgcgaattaattctgtg
gaatgtgtgtcagttagggtgtggaaagtccccaggctccccagcaggcagaagtatgcaaagcatgc
atctcaattagtcagcaaccaggtgtggaaagtccccaggctccccagcaggcagaagtatgcaaagc
atgcatctcaattagtcagcaaccatagtcccgcccctaactccgcccatcccgcccctaactccgcc
cagttccgcccattctccgccccatggctgactaattttttttatttatgcagaggccgaggccgcct
ctgcctctgagctattccagaagtagtgaggaggcttttttggaggcctaggcttttgcaaaaagctc
ccgggagcttgtatatccattttcggatctgatcaagagacaggatgaggatcgtttcgcatgattga
acaagatggattgcacgcaggttctccggccgcttgggtggagaggctattcggctatgactgggcac
aacagacaatcggctgctctgatgccgccgtgttccggctgtcagcgcaggggcgcccggttcttttt
gtcaagaccgacctgtccggtgccctgaatgaactgcaggacgaggcagcgcggctatcgtggctggc
cacgacgggcgttccttgcgcagctgtgctcgacgttgtcactgaagcgggaagggactggctgctat
tgggcgaagtgccggggcaggatctcctgtcatctcaccttgctcctgccgagaaagtatccatcatg
gctgatgcaatgcggcggctgcatacgcttgatccggctacctgcccattcgaccaccaagcgaaaca
tcgcatcgagcgagcacgtactcggatggaagccggtcttgtcgatcaggatgatctggacgaagagc
atcaggggctcgcgccagccgaactgttcgccaggctcaaggcgcgcatgcccgacggcgaggatctc
gtcgtgacccatggcgatgcctgcttgccgaatatcatggtggaaaatggccgcttttctggattcat
cgactgtggccggctgggtgtggcggaccgctatcaggacatagcgttggctacccgtgatattgctg
aagagcttggcggcgaatgggctgaccgcttcctcgtgctttacggtatcgccgctcccgattcgcag
cgcatcgccttctatcgccttcttgacgagttcttctgagcgggactctggggttcgaaatgaccgac
caagcgacgcccaacctgccatcacgagatttcgattccaccgccgccttctatgaaaggttgggctt
cggaatcgttttccgggacgccggctggatgatcctccagcgcggggatctcatgctggagttcttcg
cccaccccaacttgtttattgcagcttataatggttacaaataaagcaatagcatcacaaatttcaca
aataaagcatttttttcactgcattctagttgtggtttgtccaaactcatcaatgtatcttatcatgt
ctgtataccgtcgacctctagctagagcttggcgtaatcatggtcatagctgtttcctgtgtgaaatt
gttatccgctcacaattccacacaacatacgagccggaagcataaagtgtaaagcctggggtgcctaa
tgagtgagctaactcacattaattgcgttgcgctcactgcccgctttccagtcgggaaacctgtcgtg
ccagctgcattaatgaatcggccaacgcgcggggagaggcggtttgcgtattgggcgctcttccgctt
cctcgctcactgactcgctgcgctcggtcgttcggctgcggcgagcggtatcagctcactcaaaggcg
gtaatacggttatccacagaatcaggggataacgcaggaaagaacatgtgagcaaaaggccagcaaaa
ggccaggaaccgtaaaaaggccgcgttgctggcgtttttccataggctccgcccccctgacgagcatc
acaaaaatcgacgctcaagtcagaggtggcgaaacccgacaggactataaagataccaggcgtttccc
cctggaagctccctcgtgcgctctcctgttccgaccctgccgcttaccggatacctgtccgcctttct
cccttcgggaagcgtggcgctttctcatagctcacgctgtaggtatctcagttcggtgtaggtcgttc
gctccaagctgggctgtgtgcacgaaccccccgttcagcccgaccgctgcgccttatccggtaactat
cgtcttgagtccaacccggtaagacacgacttatcgccactggcagcagccactggtaacaggattag
cagagcgaggtatgtaggcggtgctacagagttcttgaagtggtggcctaactacggctacactagaa
gaacagtatttggtatctgcgctctgctgaagccagttaccttcggaaaaagagttggtagctcttga
tccggcaaacaaaccaccgctggtagcggtggtttttttgtttgcaagcagcagattacgcgcagaaa
aaaaggatctcaagaagatcctttgatcttttctacggggtctgacgctcagtggaacgaaaactcac
gttaagggattttggtcatgagattatcaaaaaggatcttcacctagatccttttaaattaaaaatga
agttttaaatcaatctaaagtatatatgagtaaacttggtctgacagttaccaatgcttaatcagtga
ggcacctatctcagcgatctgtctatttcgttcatccatagttgcctgactccccgtcgtgtagataa
ctacgatacgggagggcttaccatctggccccagtgctgcaatgataccgcgagacccacgctcaccg
gctccagatttatcagcaataaaccagccagccggaagggccgagcgcagaagtggtcctgcaacttt
atccgcctccatccagtctattaattgttgccgggaagctagagtaagtagttcgccagttaatagtt
tgcgcaacgttgttgccattgctacaggcatcgtggtgtcacgctcgtcgtttggtatggcttcattc
agctccggttcccaacgatcaaggcgagttacatgatcccccatgttgtgcaaaaaagcggttagctc
cttcggtcctccgatcgttgtcagaagtaagttggccgcagtgttatcactcatggttatggcagcac
tgcataattctcttactgtcatgccatccgtaagatgcttttctgtgactggtgagtactcaaccaag
tcattctgagaatagtgtatgcggcgaccgagttgctcttgcccggcgtcaatacgggataataccgc
gccacatagcagaactttaaaagtgctcatcattggaaaacgttcttcggggcgaaaactctcaagga
tcttaccgctgttgagatccagttcgatgtaacccactcgtgcacccaactgatcttcagcatctttt
actttcaccagcgtttctgggtgagcaaaaacaggaaggcaaaatgccgcaaaaaagggaataagggc
gacacggaaatgttgaatactcatactcttcctttttcaatattattgaagcatttatcagggttatt
gtctcatgagcggatacatatttgaatgtatttagaaaaataaacaaataggggttccgcgcacattt
ccccgaaaagtgccacctgacgtc
pZF91_tPA_BA2_RBD_tri_mRNA
(SEQ ID NO: 125)
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATGGACGCCATGAAGAGAGGCCTGTGCTGCGTGCTGCTGCTGTGCGGCGCCGTGTTCGTGAG
CCCTAGCAGGGTCCAACCAACAGAGAGCATTGTGAGGTTTCCAAACATCACCAACCTGTGTCCATTTG
acGAGGTGTTCAAcGCCACCAGGTTTGCCTCTGTCTATGCCTGGAACAGGAAGAGGATTAGCAACTGT
GTGGCTGACTACTCTGTGCTCTACAACTTCGCCcCCTTCttCGCCTTCAAGTGTTATGGAGTGAGCCC
AACCAAACTGAATGACCTGTGTTTCACCAATGTCTATGCTGACTCCTTTGTGATTAGGGGAAACGAGG
TGAGCCAGATTGCCCCTGGACAAACAGGCAAcATTGCTGACTACAACTACAAACTGCCTGATGACTTC
ACAGGCTGTGTGATTGCCTGGAACAGCAACAAgCTGGACAGCAAGGTGGGCGGCAACTACAACTACCT
CTACAGACTGTTCAGGAAGAGCAACCTGAAACCATTTGAGAGGGACATCAGCACAGAGATTTACCAGG
CTGGCAaCAagCCATGTAATGGAGTGGccGGCTTCAACTGTTACTTTCCACTCCggTCCTATGGCTTC
CggCCAACCtacGGAGTGGGCcACCAACCATACAGGGTGGTGGTGCTGTCCTTTGAACTGCTCCATGC
CCCTGCCACAGTGTGTGGACCAAAGAAGAGCACCAACCTGGTGAAGAACAAGTGTGTGAACTTCGGCA
GCGGCGGCTACATCCCCGAGGCCCCTAGAGACGGCCAAGCCTACGTGAGAAAGGACGGCGAATGGGTA
CTCCTGAGCACCTTCCTGGGTGGCGGAGGCAGCGGCGGGGGAGGCAGCTGATAATAGCTCGAGCTGGT
ACTGCATGCACGCAATGCTAGCTGCCCCTTTCCCGTCCTGGGTACCCCGAGTCTCCCCCGACCTCGGG
TCCCAGGTATGCTCCCACCTCCACCTGCCCCACTCACCACCTCTGCTAGTTCCAGACACCTCCCAAGC
ACGCAGCAATGCAGCTCAAAACGCTTAGCCTAGCCACACCCCCACGGGAAACAGCAGTGATTAACCTT
TAGCAATAAACGAAAGTTTAACTAAGCTATACTAACCCCAGGGTTGGTCAATTTCGTGCCAGCCACAC
CCTGGAGCTAGCaaaccgctgatcagcctcgactgtgccttctagttgccagccatctgttgtttgcc
cctcccccgtgccttccttgaccctggaaggtgccactcccactgtcctttcctaataaaatgaggaa
attgcatcgcattgtctgagtaggtgtcattctattctggggggtggggtggggcaggacagcaaggg
ggaggattgggaagacaatagcaggcatgctggggatgcggtgggctctatggcttctgaggcggaaa
gaaccagctggggctctagggggtatccccacgcgccctgtagcggcgcattaagcgcggcgggtgtg
gtggttacgcgcagcgtgaccgctacacttgccagcgccctagcgcccgctcctttcgctttcttccc
ttcctttctcgccacgttcgccggctttccccgtcaagctctaaatcgggggctccctttagggttcc
gatttagtgctttacggcacctcgaccccaaaaaacttgattagggtgatggttcacgtagtgggcca
tcgccctgatagacggtttttcgccctttgacgttggagtccacgttctttaatagtggactcttgtt
ccaaactggaacaacactcaaccctatctcggtctattcttttgatttataagggattttgccgattt
cggcctattggttaaaaaatgagctgatttaacaaaaatttaacgcgaattaattctgtggaatgtgt
gtcagttagggtgtggaaagtccccaggctccccagcaggcagaagtatgcaaagcatgcatctcaat
tagtcagcaaccaggtgtggaaagtccccaggctccccagcaggcagaagtatgcaaagcatgcatct
caattagtcagcaaccatagtcccgcccctaactccgcccatcccgcccctaactccgcccagttccg
cccattctccgccccatggctgactaattttttttatttatgcagaggccgaggccgcctctgcctct
gagctattccagaagtagtgaggaggcttttttggaggcctaggcttttgcaaaaagctcccgggagc
ttgtatatccattttcggatctgatcaagagacaggatgaggatcgtttcgcatgattgaacaagatg
gattgcacgcaggttctccggccgcttgggtggagaggctattcggctatgactgggcacaacagaca
atcggctgctctgatgccgccgtgttccggctgtcagcgcaggggcgcccggttctttttgtcaagac
cgacctgtccggtgccctgaatgaactgcaggacgaggcagcgcggctatcgtggctggccacgacgg
gcgttccttgcgcagctgtgctcgacgttgtcactgaagcgggaagggactggctgctattgggcgaa
gtgccggggcaggatctcctgtcatctcaccttgctcctgccgagaaagtatccatcatggctgatgc
aatgcggcggctgcatacgcttgatccggctacctgcccattcgaccaccaagcgaaacatcgcatcg
agcgagcacgtactcggatggaagccggtcttgtcgatcaggatgatctggacgaagagcatcagggg
ctcgcgccagccgaactgttcgccaggctcaaggcgcgcatgcccgacggcgaggatctcgtcgtgac
ccatggcgatgcctgcttgccgaatatcatggtggaaaatggccgcttttctggattcatcgactgtg
gccggctgggtgtggcggaccgctatcaggacatagcgttggctacccgtgatattgctgaagagctt
ggcggcgaatgggctgaccgcttcctcgtgctttacggtatcgccgctcccgattcgcagcgcatcgc
cttctatcgccttcttgacgagttcttctgagcgggactctggggttcgaaatgaccgaccaagcgac
gcccaacctgccatcacgagatttcgattccaccgccgccttctatgaaaggttgggcttcggaatcg
ttttccgggacgccggctggatgatcctccagcgcggggatctcatgctggagttcttcgcccacccc
aacttgtttattgcagcttataatggttacaaataaagcaatagcatcacaaatttcacaaataaagc
atttttttcactgcattctagttgtggtttgtccaaactcatcaatgtatcttatcatgtctgtatac
cgtcgacctctagctagagcttggcgtaatcatggtcatagctgtttcctgtgtgaaattgttatccg
ctcacaattccacacaacatacgagccggaagcataaagtgtaaagcctggggtgcctaatgagtgag
ctaactcacattaattgcgttgcgctcactgcccgctttccagtcgggaaacctgtcgtgccagctgc
attaatgaatcggccaacgcgcggggagaggcggtttgcgtattgggcgctcttccgcttcctcgctc
actgactcgctgcgctcggtcgttcggctgcggcgagcggtatcagctcactcaaaggcggtaatacg
gttatccacagaatcaggggataacgcaggaaagaacatgtgagcaaaaggccagcaaaaggccagga
accgtaaaaaggccgcgttgctggcgtttttccataggctccgcccccctgacgagcatcacaaaaat
cgacgctcaagtcagaggtggcgaaacccgacaggactataaagataccaggcgtttccccctggaag
ctccctcgtgcgctctcctgttccgaccctgccgcttaccggatacctgtccgcctttctcccttcgg
gaagcgtggcgctttctcatagctcacgctgtaggtatctcagttcggtgtaggtcgttcgctccaag
ctgggctgtgtgcacgaaccccccgttcagcccgaccgctgcgccttatccggtaactatcgtcttga
gtccaacccggtaagacacgacttatcgccactggcagcagccactggtaacaggattagcagagcga
ggtatgtaggcggtgctacagagttcttgaagtggtggcctaactacggctacactagaagaacagta
tttggtatctgcgctctgctgaagccagttaccttcggaaaaagagttggtagctcttgatccggcaa
acaaaccaccgctggtagcggtggtttttttgtttgcaagcagcagattacgcgcagaaaaaaaggat
ctcaagaagatcctttgatcttttctacggggtctgacgctcagtggaacgaaaactcacgttaaggg
attttggtcatgagattatcaaaaaggatcttcacctagatccttttaaattaaaaatgaagttttaa
atcaatctaaagtatatatgagtaaacttggtctgacagttaccaatgcttaatcagtgaggcaccta
tctcagcgatctgtctatttcgttcatccatagttgcctgactccccgtcgtgtagataactacgata
cgggagggcttaccatctggccccagtgctgcaatgataccgcgagacccacgctcaccggctccaga
tttatcagcaataaaccagccagccggaagggccgagcgcagaagtggtcctgcaactttatccgcct
ccatccagtctattaattgttgccgggaagctagagtaagtagttcgccagttaatagtttgcgcaac
gttgttgccattgctacaggcatcgtggtgtcacgctcgtcgtttggtatggcttcattcagctccgg
ttcccaacgatcaaggcgagttacatgatcccccatgttgtgcaaaaaagcggttagctccttcggtc
ctccgatcgttgtcagaagtaagttggccgcagtgttatcactcatggttatggcagcactgcataat
tctcttactgtcatgccatccgtaagatgcttttctgtgactggtgagtactcaaccaagtcattctg
agaatagtgtatgcggcgaccgagttgctcttgcccggcgtcaatacgggataataccgcgccacata
gcagaactttaaaagtgctcatcattggaaaacgttcttcggggcgaaaactctcaaggatcttaccg
ctgttgagatccagttcgatgtaacccactcgtgcacccaactgatcttcagcatcttttactttcac
cagcgtttctgggtgagcaaaaacaggaaggcaaaatgccgcaaaaaagggaataagggcgacacgga
aatgttgaatactcatactcttcctttttcaatattattgaagcatttatcagggttattgtctcatg
agcggatacatatttgaatgtatttagaaaaataaacaaataggggttccgcgcacatttccccgaaa
agtgccacctgacgtc
Furin Cleavage
SEQ ID NO: 11
GSAS
fibritin Foldon
SEQ ID NO: 12
GYIPEAPRDGQAYVRKDGEWVLLSTF
Spy Tag
SEQ ID NO: 13
RGVPHIVMVDAYKRYK
SPY Catcher
SEQ ID NO: 14
VTTLSGLSGEQGPSGDMTTEEDSATHIKFSKRDEDGRELAGATMELRDSSGKTISTWISDGHVKDFYL
YPGKYTFVETAAPDGYEVATPIEFTVNEDGQVTVDGEATEGDAHT
Cleavage
SEQ ID NO: 15
RRAR
Cleavage
SEQ ID NO: 16
GSSS
Cleavage
SEQ ID NO: 17
GSGS
Omicron Spike Hexapro
SEQ ID NO: 46
FVFLVLLPLVSSQCVNLTTRTQLPPAYTNSFTRGVYYPDKVFRSSVLHSTQDLFLPFFSNVTWFHVIS
GTNGTKRFDNPVLPFNDGVYFASIEKSNIIRGWIFGTTLDSKTQSLLIVNNATNVVIKVCEFQFCNDP
FLDHKNNKSWMESEFRVYSSANNCTFEYVSQPFLMDLEGKQGNFKNLREFVFKNIDGYFKIYSKHTPI
IVREPEDLPQGFSALEPLVDLPIGINITRFQTLLALHRSYLTPGDSSSGWTAGAAAYYVGYLQPRTFL
LKYNENGTITDAVDCALDPLSETKCTLKSFTVEKGIYQTSNFRVQPTESIVRFPNITNLCPFDEVFNA
TRFASVYAWNRKRISNCVADYSVLYNLAPFFTFKCYGVSPTKLNDLCFTNVYADSFVIRGDEVRQIAP
GQTGNIADYNYKLPDDFTGCVIAWNSNKLDSKVSGNYNYLYRLFRKSNLKPFERDISTEIYQAGNKPC
NGVAGFNCYFPLRSYSFRPTYGVGHQPYRVVVLSFELLHAPATVCGPKKSTNLVKNKCVNFNFNGLKG
TGVLTESNKKFLPFQQFGRDIADTTDAVRDPQTLEILDITPCSFGGVSVITPGTNTSNQVAVLYQGVN
CTEVPVAIHADQLTPTWRVYSTGSNVFQTRAGCLIGAEYVNNSYECDIPIGAGICASYQTQTKSHGSA
SSVASQSIIAYTMSLGAENSVAYSNNSIAIPTNFTISVTTEILPVSMTKTSVDCTMYICGDSTECSNL
LLQYGSFCTQLKRALTGIAVEQDKNTQEVFAQVKQIYKTPPIKYFGGFNFSQILPDPSKPSKRSPIED
LLFNKVTLADAGFIKQYGDCLGDIAARDLICAQKFKGLTVLPPLLTDEMIAQYTSALLAGTITSGWTF
GAGPALQIPFPMQMAYRFNGIGVTQNVLYENQKLIANQFNSAIGKIQDSLSSTPSALGKLQDVVNHNA
QALNTLVKQLSSKFGAISSVLNDIFSRLDPPEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQPELDSFKEELDKYFKNHT
SPDVDLGDISGINASVVNIQKEIDRLNEVAKNLNESLIDLQELGKYEQYIKWPWYIWLGFIAGLIAIV
MVTIMLCCMTSCCSCLKGCCSCGSCCKFDEDDSEPVLKGVKLHYT*
pAZ50-SARS-CoV-2 HexaPro-FIS-a
SEQ ID NO: 70
gtcgacattgattattgactagttattaatagtaatcaattacggggtcattagttcatagcccatat
atggagttccgcgttacataacttacggtaaatggcccgcctggctgaccgcccaacgacccccgccc
attgacgtcaataatgacgtatgttcccatagtaacgccaatagggactttccattgacgtcaatggg
tggactatttacggtaaactgcccacttggcagtacatcaagtgtatcatatgccaagtacgccccct
attgacgtcaatgacggtaaatggcccgcctggcattatgcccagtacatgaccttatgggactttcc
tacttggcagtacatctacgtattagtcatcgctattaccatgggtcgaggtgagccccacgttctgc
ttcactctccccatctcccccccctccccacccccaattttgtatttatttattttttaattattttg
tgcagcgatgggggcggggggggggggggcgcgcgccaggcggggcggggcggggcgaggggcggggc
ggggcgaggcggagaggtgcggcggcagccaatcagagcggcgcgctccgaaagtttccttttatggc
gaggcggcggcggcggcggccctataaaaagcgaagcgcgcggcgggcgggagtcgctgcgttgcctt
cgccccgtgccccgctccgcgccgcctcgcgccgcccgccccggctctgactgaccgcgttactccca
caggtgagcgggcgggacggcccttctcctccgggctgtaattagcgcttggtttaatgacggctcgt
ttcttttctgtggctgcgtgaaagccttaaagggctccgggagggccctttgtgcgggggggagcggc
tcggggggtgcgtgcgtgtgtgtgtgcgtggggagcgccgcgtgcggcccgcgctgcccggcggctgt
gagcgctgcgggcgcggcgcggggctttgtgcgctccgcgtgtgcgcgaggggagcgcggccgggggc
ggtgccccgcggtgcgggggggctgcgaggggaacaaaggctgcgtgcggggtgtgtgcgtggggggg
tgagcagggggtgtgggcgcggcggtcgggctgtaacccccccctgcacccccctccccgagttgctg
agcacggcccggcttcgggtgcggggctccgtgcggggcgtggcgcggggctcgccgtgccgggcggg
gggtggcggcaggtgggggtgccgggcggggcggggccgcctcgggccggggagggctcgggggaggg
gcgcggcggcccccggagcgccggcggctgtcgaggcgcggcgagccgcagccattgccttttatggt
aatcgtgcgagagggcgcagggacttcctttgtcccaaatctgtgcggagccgaaatctgggaggcgc
cgccgcaccccctctagcgggcgcggggcgaagcggtgcggcgccggcaggaaggaaatgggcgggga
gggccttcgtgcgtcgccgcgccgccgtccccttctccctctccagcctcggggctgtccgcgggggg
acggctgccttcgggggggacggggcagggcggggttcggcttctggcgtgtgaccggcggctctagc
gcctctgctaaccatgttcatgccttcttctttttcctacagctcctgggcaacgtgctggttgttgt
gctgtctcatcattttggcaaagaattgcggccgtctcaggccgagttcggtaccgccaccatgttcg
tgttcctggtgctcctgcctctggtgagcagccagtgcgtgaacctgaccacccgaacccagctccca
ccagcctacaccaacagctttacacggggcgtgtactaccctgacaaggtgttcagatctagcgtcct
gcacagcactcaggacctcttcctgccgttcttcagcaacgtgacatggttccacgccatccacgtga
gcggcacaaacggaaccaagcggtttgataaccccgtcctgccattcaatgatggagtttacttcgcc
agtaccgagaagagtaacatcatccggggctggatcttcggcaccaccctggatagcaaaacacagag
cctcctgatcgtgaacaatgccacgaacgtcgtgatcaaggtgtgcgagttccagttttgcaatgatc
ctttcctgggtgtgtactaccacaagaacaacaagagctggatggaaagcgagttcagagtctacagc
agcgccaacaactgcacattcgagtacgtctctcagccttttctgatggaccttgaggggaaacaagg
caacttcaagaacctgagagaattcgtgttcaagaacatcgacggctacttcaaaatctactccaagc
acacacccatcaacctggtccgggacctccctcagggcttcagcgccctggaacccctggtcgacctg
cccataggcatcaacataacgcggttccaaaccctgctggccctgcatagatcctacctgactcctgg
cgacagcagcagcggatggaccgccggagctgcagcctactatgtgggctacctgcaacctagaacct
tcctgctgaagtacaacgagaacggcacaatcacagacgccgtcgactgcgccctggaccctctctct
gagacaaagtgcaccctgaagtccttcaccgtggaaaagggcatctaccagaccagcaacttccgggt
gcagcctacagagagcatcgtgcgatttccaaacattaccaacctctgccccttcggcgaggtgttta
acgccacaagatttgcctccgtttacgcctggaatagaaagagaatcagcaattgtgtggccgactac
tccgtgctgtataacagcgcctctttcagcaccttcaagtgctacggcgtttccccaacaaagctgaa
tgacctgtgcttcaccaacgtgtacgccgactccttcgtaattagaggcgatgaggtgcggcagatcg
caccaggccagaccggtaagatcgctgactacaactataagctgcctgatgattttacaggctgcgtg
atcgcctggaactctaacaacctggatagcaaggtgggcggcaactacaactacctgtaccggctgtt
tcgcaagtctaacctgaaacctttcgagagagacatctccacagagatctaccaggccggttctacac
cttgtaacggggtggaaggcttcaactgttacttccctctgcaaagctacggcttccagcctaccaat
ggagtcggctaccagccataccgggtggtcgtgctgtccttcgagttactccacgcccccgccaccgt
ctgcggtcctaagaagtccaccaatctggttaagaacaaatgcgtgaacttcaacttcaacggcctga
ccgggaccggcgtgctgaccgaaagcaacaaaaagttcctccccttccagcagttcggccgtgatatc
gctgacaccacagatgccgtcagagatccacagaccctggaaatcctggatattacaccctgctcctt
cggaggagtttctgtgatcacccccgggaccaataccagcaaccaggtggctgtgctgtaccaagatg
ttaactgcaccgaggttcctgtggccatccacgccgatcagctgacacctacttggagagtgtactcc
actggctccaatgtgttccagaccagggccggatgtctgatcggcgccgagcacgtgaataacagtta
cgagtgcgacatccctatcggcgccggcatctgtgccagctaccagacccagacaaacagccctgggt
ctgcttcctctgtagctagccagagcatcatcgcctacaccatgagcctgggcgcagagaacagcgtg
gcctattccaacaactctatcgccattcccaccaactttacaattagcgtcacaacagagatcctgcc
cgtgagcatgaccaagaccagcgtggactgtacaatgtacatctgtggcgacagcactgaatgcagca
acctgctgctgcaatacggctccttttgcacccaactgaaccgggcgctgaccggaatcgccgtggaa
caggacaaaaatacccaggaggtgttcgcccaagtgaagcagatctacaagaccccacctatcaagga
cttcggcggctttaactttagccagattctccctgatccttctaagcctagcaagcggagccctatcg
aggatctgctgttcaacaaggtcaccctggccgatgccggctttatcaaacagtatggcgattgcctg
ggcgacatagccgccagagatctgatctgcgcccagaaattcaacggcctgacagttctcccacctct
gctgaccgacgagatgatcgctcagtacacctctgccctgctggctggcaccatcacatctgggtgga
catttggcgccggccccgccctgcagatcccctttcccatgcagatggcctatagattcaacggaatc
ggcgtgacccagaacgtgctgtatgaaaaccagaagctgatcgctaaccagttcaattctgccatcgg
caagatccaggactccctctcctctacccccagcgccctgggcaaactgcaggacgtggtgaatcaga
acgcccaagccctgaacaccctggtgaagcagctcagcagcaattttggcgccatcagctctgtgctg
aacgatatcctgtctagactggaccctccagaagccgaagtccagatcgatagactgatcacaggcag
actgcagtccctgcaaacctacgtgacccaacagctgatcagggccgctgaaataagagccagcgcca
atctcgccgctaccaagatgtccgagtgtgtgctgggacagtctaaacgcgttgacttctgcggcaaa
ggctatcacctgatgagcttcccccagagcgcgccgcacggcgtggtgttcctgcatgtgacatacgt
gcctgcccaagagaagaatttcacaaccgcccctgccatctgccacgacggcaaggcccacttcccaa
gagagggcgttttcgtttccaatggcacacactggttcgtgacacaaagaaacttctacgaaccccag
attatcaccaccgacaacaccttcgtgagtggcaattgtgacgtggtcatcggaatcgtgaacaacac
agtgtacgaccctctgcaacctgagctggactcttttaaggaagagctggacaagtactttaaaaacc
acaccagccccgatgtggacctgggcgacatcagtggcattaacgccagcgtggtgaacatccaaaag
gaaatcgacagactgaacgaggtggccaagaacctgaacgagtccctgatcgacctgcaggagctcgg
caaatacgagcagTACATCAAGTGGCCATGGTACATCTGGCTGGGCTTCATTGCTGGACTGATTGCCA
TTGTGATGGTGACCATAATGCTGTGTTGTATGACCTCCTGTTGTTCCTGTCTGAAAGGCTGTTGTTCC
TGTGGCTCCTGTTGTAAGTTTGATGAGGATGACTCTGAACCTGTGCTGAAAGGAGTGAAACTGCACTA
CACCTGActcgagcgataattcactcctcaggtgcaggctgcctatcagaaggtggtggctggtgtgg
ccaatgccctggctcacaaataccactgagatctttttccctctgccaaaaattatggggacatcatg
aagccccttgagcatctgacttctggctaataaaggaaatttattttcattgcaatagtgtgttggaa
ttttttgtgtctctcactcggaaggacatatgggagggcaaatcatttaaaacatcagaatgagtatt
tggtttagagtttggcaacatatgccatatgctggctgccatgaacaaaggtggctataaagaggtca
tcagtatatgaaacagccccctgctgtccattccttattccatagaaaagccttgacttgaggttaga
ttttttttatattttgttttgtgttatttttttctttaacatccctaaaattttccttacatgtttta
ctagccagatttttcctcctctcctgactactcccagtcatagctgtccctcttctcttatgaagatc
cctcgacctgcagcccaagcttggcgtaatcatggtcatagctgtttcctgtgtgaaattgttatccg
ctcacaattccacacaacatacgagccggaagcataaagtgtaaagcctggggtgcctaatgagtgag
ctaactcacattaattgcgttgcgctcactgcccgctttccagtcgggaaacctgtcgtgccagcgga
tcgatccgctgcattaatgaatcggccaacgcgcggggagaggcggtttgcgtattgggcgctcttcc
gcttcctcgctcactgactcgctgcgctcggtcgttcggctgcggcgagcggtatcagctcactcaaa
ggcggtaatacggttatccacagaatcaggggataacgcaggaaagaacatgtgagcaaaaggccagc
aaaaggccaggaaccgtaaaaaggccgcgttgctggcgtttttccataggctccgcccccctgacgag
catcacaaaaatcgacgctcaagtcagaggtggcgaaacccgacaggactataaagataccaggcgtt
tccccctggaagctccctcgtgcgctctcctgttccgaccctgccgcttaccggatacctgtccgcct
ttctcccttcgggaagcgtggcgctttctcaatgctcacgctgtaggtatctcagttcggtgtaggtc
gttcgctccaagctgggctgtgtgcacgaaccccccgttcagcccgaccgctgcgccttatccggtaa
ctatcgtcttgagtccaacccggtaagacacgacttatcgccactggcagcagccactggtaacagga
ttagcagagcgaggtatgtaggcggtgctacagagttcttgaagtggtggcctaactacggctacact
agaaggacagtatttggtatctgcgctctgctgaagccagttaccttcggaaaaagagttggtagctc
ttgatccggcaaacaaaccaccgctggtagcggtggtttttttgtttgcaagcagcagattacgcgca
gaaaaaaaggatctcaagaagatcctttgatcttttctacggggtctgacgctcagtggaacgaaaac
tcacgttaagggattttggtcatgagattatcaaaaaggatcttcacctagatccttttaaattaaaa
atgaagttttaaatcaatctaaagtatatatgagtaaacttggtctgacagttaccaatgcttaatca
gtgaggcacctatctcagcgatctgtctatttcgttcatccatagttgcctgactccccgtcgtgtag
ataactacgatacgggagggcttaccatctggccccagtgctgcaatgataccgcgagacccacgctc
accggctccagatttatcagcaataaaccagccagccggaagggccgagcgcagaagtggtcctgcaa
ctttatccgcctccatccagtctattaattgttgccgggaagctagagtaagtagttcgccagttaat
agtttgcgcaacgttgttgccattgctacaggcatcgtggtgtcacgctcgtcgtttggtatggcttc
attcagctccggttcccaacgatcaaggcgagttacatgatcccccatgttgtgcaaaaaagcggtta
gctccttcggtcctccgatcgttgtcagaagtaagttggccgcagtgttatcactcatggttatggca
gcactgcataattctcttactgtcatgccatccgtaagatgcttttctgtgactggtgagtactcaac
caagtcattctgagaatagtgtatgcggcgaccgagttgctcttgcccggcgtcaatacgggataata
ccgcgccacatagcagaactttaaaagtgctcatcattggaaaacgttcttcggggcgaaaactctca
aggatcttaccgctgttgagatccagttcgatgtaacccactcgtgcacccaactgatcttcagcatc
ttttactttcaccagcgtttctgggtgagcaaaaacaggaaggcaaaatgccgcaaaaaagggaataa
gggcgacacggaaatgttgaatactcatactcttcctttttcaatattattgaagcatttatcagggt
tattgtctcatgagcggatacatatttgaatgtatttagaaaaataaacaaataggggttccgcgcac
atttccccgaaaagtgccacctg
pAZ51-SARS-CoV-2 HexaPro(ECD)-HA(TM CT)-2A-M1(H5N1)-a
SEQ ID NO: 71
gtcgacattgattattgactagttattaatagtaatcaattacggggtcattagttcatagcccatat
atggagttccgcgttacataacttacggtaaatggcccgcctggctgaccgcccaacgacccccgccc
attgacgtcaataatgacgtatgttcccatagtaacgccaatagggactttccattgacgtcaatggg
tggactatttacggtaaactgcccacttggcagtacatcaagtgtatcatatgccaagtacgccccct
attgacgtcaatgacggtaaatggcccgcctggcattatgcccagtacatgaccttatgggactttcc
tacttggcagtacatctacgtattagtcatcgctattaccatgggtcgaggtgagccccacgttctgc
ttcactctccccatctcccccccctccccacccccaattttgtatttatttattttttaattattttg
tgcagcgatgggggcggggggggggggggcgcgcgccaggcggggcggggcggggcgaggggcggggc
ggggcgaggcggagaggtgcggcggcagccaatcagagcggcgcgctccgaaagtttccttttatggc
gaggcggcggcggcggcggccctataaaaagcgaagcgcgcggcgggcgggagtcgctgcgttgcctt
cgccccgtgccccgctccgcgccgcctcgcgccgcccgccccggctctgactgaccgcgttactccca
caggtgagcgggcgggacggcccttctcctccgggctgtaattagcgcttggtttaatgacggctcgt
ttcttttctgtggctgcgtgaaagccttaaagggctccgggagggccctttgtgcgggggggagcggc
tcggggggtgcgtgcgtgtgtgtgtgcgtggggagcgccgcgtgcggcccgcgctgcccggcggctgt
gagcgctgcgggcgcggcgcggggctttgtgcgctccgcgtgtgcgcgaggggagcgcggccgggggc
ggtgccccgcggtgcgggggggctgcgaggggaacaaaggctgcgtgcggggtgtgtgcgtggggggg
tgagcagggggtgtgggcgcggcggtcgggctgtaacccccccctgcacccccctccccgagttgctg
agcacggcccggcttcgggtgcggggctccgtgcggggcgtggcgcggggctcgccgtgccgggcggg
gggtggcggcaggtgggggtgccgggcggggcggggccgcctcgggccggggagggctcgggggaggg
gcgcggcggcccccggagcgccggcggctgtcgaggcgcggcgagccgcagccattgccttttatggt
aatcgtgcgagagggcgcagggacttcctttgtcccaaatctgtgcggagccgaaatctgggaggcgc
cgccgcaccccctctagcgggcgcggggcgaagcggtgcggcgccggcaggaaggaaatgggcgggga
gggccttcgtgcgtcgccgcgccgccgtccccttctccctctccagcctcggggctgtccgcgggggg
acggctgccttcgggggggacggggcagggcggggttcggcttctggcgtgtgaccggcggctctagc
gcctctgctaaccatgttcatgccttcttctttttcctacagctcctgggcaacgtgctggttgttgt
gctgtctcatcattttggcaaagaattgcggccgtctcaggccgagttcggtaccgccaccatgttcg
tgttcctggtgctcctgcctctggtgagcagccagtgcgtgaacctgaccacccgaacccagctccca
ccagcctacaccaacagctttacacggggcgtgtactaccctgacaaggtgttcagatctagcgtcct
gcacagcactcaggacctcttcctgccgttcttcagcaacgtgacatggttccacgccatccacgtga
gcggcacaaacggaaccaagcggtttgataaccccgtcctgccattcaatgatggagtttacttcgcc
agtaccgagaagagtaacatcatccggggctggatcttcggcaccaccctggatagcaaaacacagag
cctcctgatcgtgaacaatgccacgaacgtcgtgatcaaggtgtgcgagttccagttttgcaatgatc
ctttcctgggtgtgtactaccacaagaacaacaagagctggatggaaagcgagttcagagtctacagc
agcgccaacaactgcacattcgagtacgtctctcagccttttctgatggaccttgaggggaaacaagg
caacttcaagaacctgagagaattcgtgttcaagaacatcgacggctacttcaaaatctactccaagc
acacacccatcaacctggtccgggacctccctcagggcttcagcgccctggaacccctggtcgacctg
cccataggcatcaacataacgcggttccaaaccctgctggccctgcatagatcctacctgactcctgg
cgacagcagcagcggatggaccgccggagctgcagcctactatgtgggctacctgcaacctagaacct
tcctgctgaagtacaacgagaacggcacaatcacagacgccgtcgactgcgccctggaccctctctct
gagacaaagtgcaccctgaagtccttcaccgtggaaaagggcatctaccagaccagcaacttccgggt
gcagcctacagagagcatcgtgcgatttccaaacattaccaacctctgccccttcggcgaggtgttta
acgccacaagatttgcctccgtttacgcctggaatagaaagagaatcagcaattgtgtggccgactac
tccgtgctgtataacagcgcctctttcagcaccttcaagtgctacggcgtttccccaacaaagctgaa
tgacctgtgcttcaccaacgtgtacgccgactccttcgtaattagaggcgatgaggtgcggcagatcg
caccaggccagaccggtaagatcgctgactacaactataagctgcctgatgattttacaggctgcgtg
atcgcctggaactctaacaacctggatagcaaggtgggcggcaactacaactacctgtaccggctgtt
tcgcaagtctaacctgaaacctttcgagagagacatctccacagagatctaccaggccggttctacac
cttgtaacggggtggaaggcttcaactgttacttccctctgcaaagctacggcttccagcctaccaat
ggagtcggctaccagccataccgggtggtcgtgctgtccttcgagttactccacgcccccgccaccgt
ctgcggtcctaagaagtccaccaatctggttaagaacaaatgcgtgaacttcaacttcaacggcctga
ccgggaccggcgtgctgaccgaaagcaacaaaaagttcctccccttccagcagttcggccgtgatatc
gctgacaccacagatgccgtcagagatccacagaccctggaaatcctggatattacaccctgctcctt
cggaggagtttctgtgatcacccccgggaccaataccagcaaccaggtggctgtgctgtaccaagatg
ttaactgcaccgaggttcctgtggccatccacgccgatcagctgacacctacttggagagtgtactcc
actggctccaatgtgttccagaccagggccggatgtctgatcggcgccgagcacgtgaataacagtta
cgagtgcgacatccctatcggcgccggcatctgtgccagctaccagacccagacaaacagccctgggt
ctgcttcctctgtagctagccagagcatcatcgcctacaccatgagcctgggcgcagagaacagcgtg
gcctattccaacaactctatcgccattcccaccaactttacaattagcgtcacaacagagatcctgcc
cgtgagcatgaccaagaccagcgtggactgtacaatgtacatctgtggcgacagcactgaatgcagca
acctgctgctgcaatacggctccttttgcacccaactgaaccgggcgctgaccggaatcgccgtggaa
caggacaaaaatacccaggaggtgttcgcccaagtgaagcagatctacaagaccccacctatcaagga
cttcggcggctttaactttagccagattctccctgatccttctaagcctagcaagcggagccctatcg
aggatctgctgttcaacaaggtcaccctggccgatgccggctttatcaaacagtatggcgattgcctg
ggcgacatagccgccagagatctgatctgcgcccagaaattcaacggcctgacagttctcccacctct
gctgaccgacgagatgatcgctcagtacacctctgccctgctggctggcaccatcacatctgggtgga
catttggcgccggccccgccctgcagatcccctttcccatgcagatggcctatagattcaacggaatc
ggcgtgacccagaacgtgctgtatgaaaaccagaagctgatcgctaaccagttcaattctgccatcgg
caagatccaggactccctctcctctacccccagcgccctgggcaaactgcaggacgtggtgaatcaga
acgcccaagccctgaacaccctggtgaagcagctcagcagcaattttggcgccatcagctctgtgctg
aacgatatcctgtctagactggaccctccagaagccgaagtccagatcgatagactgatcacaggcag
actgcagtccctgcaaacctacgtgacccaacagctgatcagggccgctgaaataagagccagcgcca
atctcgccgctaccaagatgtccgagtgtgtgctgggacagtctaaacgcgttgacttctgcggcaaa
ggctatcacctgatgagcttcccccagagcgcgccgcacggcgtggtgttcctgcatgtgacatacgt
gcctgcccaagagaagaatttcacaaccgcccctgccatctgccacgacggcaaggcccacttcccaa
gagagggcgttttcgtttccaatggcacacactggttcgtgacacaaagaaacttctacgaaccccag
attatcaccaccgacaacaccttcgtgagtggcaattgtgacgtggtcatcggaatcgtgaacaacac
agtgtacgaccctctgcaacctgagctggactcttttaaggaagagctggacaagtactttaaaaacc
acaccagccccgatgtggacctgggcgacatcagtggcattaacgccagcgtggtgaacatccaaaag
gaaatcgacagactgaacgaggtggccaagaacctgaacgagtccctgatcgacctgcaggagctcgg
caaatacgagcagTACATCAAGTGGCCACAAATACTGTCAATTTATTCAACAGTGGCGAGTTCCCTAG
CACTGGCAATCATGATGGCTGGTCTATCTTTATGGATGTGCTCCAATGGATCGTTACAATGCAGAATT
TGCATTgagggcagaggaagtctgctaacatgcggtgacgtcgaggagaatcctggcccaATGAGTCT
TCTAACCGAGGTCGAAACGTACGTTCTCTCTATCATCCCGTCAGGCCCCCTCAAAGCCGAGATCGCGC
AGAAACTTGAAGATGTCTTTGCAGGAAAGAACACCGATCTCGAGGCTCTCATGGAGTGGCTGAAGACA
AGACCAATCCTGTCACCTCTGACTAAAGGGATTTTGGGATTTGTATTCACGCTCACCGTGCCCAGTGA
GCGAGGACTGCAGCGTAGACGCTTTGTCCAGAATGCCCTAAATGGAAATGGAGATCCAAATAATATGG
ATAGGGCAGTTAAGCTATATAAGAAGCTGAAAAGAGAAATAACATTCCATGGGGCTAAAGAGGTTTCA
CTCAGCTACTCAACCGGTGCACTTGCCAGTTGCATGGGTCTCATATACAACAGGATGGGAACGGTGAC
TACGGAAGTGGCTTTTGGCCTAGTGTGTGCCACTTGTGAGCAGATTGCAGATTCACAGCATCGGTCTC
ACAGGCAGATGGCAACTATCACCAACCCACTAATCAGGCATGAAAACAGAATGGTGCTGGCCAGCACT
ACAGCTAAGGCTATGGAGCAGATGGCGGGATCAAGTGAGCAGGCAGCGGAAGCCATGGAGGTCGCTAA
TCAGGCTAGGCAGATGGTGCAGGCAATGAGGACAATTGGAACTCATCCTAACTCTAGTGCTGGTCTGA
GAGATAATCTTCTTGAAAATTTGCAGGCCTACCAGAAACGAATGGGAGTGCAGATGCAGCGATTCAAG
TGActcgagcgataattcactcctcaggtgcaggctgcctatcagaaggtggtggctggtgtggccaa
tgccctggctcacaaataccactgagatctttttccctctgccaaaaattatggggacatcatgaagc
cccttgagcatctgacttctggctaataaaggaaatttattttcattgcaatagtgtgttggaatttt
ttgtgtctctcactcggaaggacatatgggagggcaaatcatttaaaacatcagaatgagtatttggt
ttagagtttggcaacatatgccatatgctggctgccatgaacaaaggtggctataaagaggtcatcag
tatatgaaacagccccctgctgtccattccttattccatagaaaagccttgacttgaggttagatttt
ttttatattttgttttgtgttatttttttctttaacatccctaaaattttccttacatgttttactag
ccagatttttcctcctctcctgactactcccagtcatagctgtccctcttctcttatgaagatccctc
gacctgcagcccaagcttggcgtaatcatggtcatagctgtttcctgtgtgaaattgttatccgctca
caattccacacaacatacgagccggaagcataaagtgtaaagcctggggtgcctaatgagtgagctaa
ctcacattaattgcgttgcgctcactgcccgctttccagtcgggaaacctgtcgtgccagcggatcga
tccgctgcattaatgaatcggccaacgcgcggggagaggcggtttgcgtattgggcgctcttccgctt
cctcgctcactgactcgctgcgctcggtcgttcggctgcggcgagcggtatcagctcactcaaaggcg
gtaatacggttatccacagaatcaggggataacgcaggaaagaacatgtgagcaaaaggccagcaaaa
ggccaggaaccgtaaaaaggccgcgttgctggcgtttttccataggctccgcccccctgacgagcatc
acaaaaatcgacgctcaagtcagaggtggcgaaacccgacaggactataaagataccaggcgtttccc
cctggaagctccctcgtgcgctctcctgttccgaccctgccgcttaccggatacctgtccgcctttct
cccttcgggaagcgtggcgctttctcaatgctcacgctgtaggtatctcagttcggtgtaggtcgttc
gctccaagctgggctgtgtgcacgaaccccccgttcagcccgaccgctgcgccttatccggtaactat
cgtcttgagtccaacccggtaagacacgacttatcgccactggcagcagccactggtaacaggattag
cagagcgaggtatgtaggcggtgctacagagttcttgaagtggtggcctaactacggctacactagaa
ggacagtatttggtatctgcgctctgctgaagccagttaccttcggaaaaagagttggtagctcttga
tccggcaaacaaaccaccgctggtagcggtggtttttttgtttgcaagcagcagattacgcgcagaaa
aaaaggatctcaagaagatcctttgatcttttctacggggtctgacgctcagtggaacgaaaactcac
gttaagggattttggtcatgagattatcaaaaaggatcttcacctagatccttttaaattaaaaatga
agttttaaatcaatctaaagtatatatgagtaaacttggtctgacagttaccaatgcttaatcagtga
ggcacctatctcagcgatctgtctatttcgttcatccatagttgcctgactccccgtcgtgtagataa
ctacgatacgggagggcttaccatctggccccagtgctgcaatgataccgcgagacccacgctcaccg
gctccagatttatcagcaataaaccagccagccggaagggccgagcgcagaagtggtcctgcaacttt
atccgcctccatccagtctattaattgttgccgggaagctagagtaagtagttcgccagttaatagtt
tgcgcaacgttgttgccattgctacaggcatcgtggtgtcacgctcgtcgtttggtatggcttcattc
agctccggttcccaacgatcaaggcgagttacatgatcccccatgttgtgcaaaaaagcggttagctc
cttcggtcctccgatcgttgtcagaagtaagttggccgcagtgttatcactcatggttatggcagcac
tgcataattctcttactgtcatgccatccgtaagatgcttttctgtgactggtgagtactcaaccaag
tcattctgagaatagtgtatgcggcgaccgagttgctcttgcccggcgtcaatacgggataataccgc
gccacatagcagaactttaaaagtgctcatcattggaaaacgttcttcggggcgaaaactctcaagga
tcttaccgctgttgagatccagttcgatgtaacccactcgtgcacccaactgatcttcagcatctttt
actttcaccagcgtttctgggtgagcaaaaacaggaaggcaaaatgccgcaaaaaagggaataagggc
gacacggaaatgttgaatactcatactcttcctttttcaatattattgaagcatttatcagggttatt
gtctcatgagcggatacatatttgaatgtatttagaaaaataaacaaataggggttccgcgcacattt
ccccgaaaagtgccacctg
pAZ52-pcDNA3-CMV-mCCL28-a
SEQ ID NO: 72
gtcgacattgattattgactagttattaatagtaatcaattacggggtcattagttcatagcccatat
atggagttccgcgttacataacttacggtaaatggcccgcctggctgaccgcccaacgacccccgccc
attgacgtcaataatgacgtatgttcccatagtaacgccaatagggactttccattgacgtcaatggg
tggactatttacggtaaactgcccacttggcagtacatcaagtgtatcatatgccaagtacgccccct
attgacgtcaatgacggtaaatggcccgcctggcattatgcccagtacatgaccttatgggactttcc
tacttggcagtacatctacgtattagtcatcgctattaccatgggtcgaggtgagccccacgttctgc
ttcactctccccatctcccccccctccccacccccaattttgtatttatttattttttaattattttg
tgcagcgatgggggcggggggggggggggcgcgcgccaggcggggcggggcggggcgaggggcggggc
ggggcgaggcggagaggtgcggcggcagccaatcagagcggcgcgctccgaaagtttccttttatggc
gaggcggcggcggcggcggccctataaaaagcgaagcgcgcggcgggcgggagtcgctgcgttgcctt
cgccccgtgccccgctccgcgccgcctcgcgccgcccgccccggctctgactgaccgcgttactccca
caggtgagcgggcgggacggcccttctcctccgggctgtaattagcgcttggtttaatgacggctcgt
ttcttttctgtggctgcgtgaaagccttaaagggctccgggagggccctttgtgcgggggggagcggc
tcggggggtgcgtgcgtgtgtgtgtgcgtggggagcgccgcgtgcggcccgcgctgcccggcggctgt
gagcgctgcgggcgcggcgcggggctttgtgcgctccgcgtgtgcgcgaggggagcgcggccgggggc
ggtgccccgcggtgcgggggggctgcgaggggaacaaaggctgcgtgcggggtgtgtgcgtggggggg
tgagcagggggtgtgggcgcggcggtcgggctgtaacccccccctgcacccccctccccgagttgctg
agcacggcccggcttcgggtgcggggctccgtgcggggcgtggcgcggggctcgccgtgccgggcggg
gggtggcggcaggtgggggtgccgggcggggcggggccgcctcgggccggggagggctcgggggaggg
gcgcggcggcccccggagcgccggcggctgtcgaggcgcggcgagccgcagccattgccttttatggt
aatcgtgcgagagggcgcagggacttcctttgtcccaaatctgtgcggagccgaaatctgggaggcgc
cgccgcaccccctctagcgggcgcggggcgaagcggtgcggcgccggcaggaaggaaatgggcgggga
gggccttcgtgcgtcgccgcgccgccgtccccttctccctctccagcctcggggctgtccgcgggggg
acggctgccttcgggggggacggggcagggcggggttcggcttctggcgtgtgaccggcggctctagc
gcctctgctaaccatgttcatgccttcttctttttcctacagctcctgggcaacgtgctggttgttgt
gctgtctcatcattttggcaaagaattgcggccgtctcaggccgagttcggtaccgccaccATGCAGC
AAGCAGGGCTCACACTCATGGCTGTGGCTGTGTGTGTGGCTTTTCAAACCTCAGAAGCCATACTTCCC
ATGGCCTCCAGCTGTTGCACTGAGGTGTCTCATCATGTTTCCGGAAGACTTCTGGAAAGAGTGAGTTC
ATGCAGCATCCAGAGAGCTGACGGGGACTGCGACCTGGCTGCTGTCATCCTTCATGTTAAACGTAGAA
GAATCTGCATCAGCCCGCACAATCGTACTTTGAAGCAGTGGATGAGAGCCTCAGAGGTAAAGAAGAAT
GGCAGAGAAAACGTATGTTCTGGGAAAAAACAACCCAGCAGGAAGGACAGAAAAGGGCACACTACGAG
AAAGCACAGAACACGTGGAACACACAGGCACGAAGCCTCTCGTTAGctcgagcgataattcactcctc
aggtgcaggctgcctatcagaaggtggtggctggtgtggccaatgccctggctcacaaataccactga
gatctttttccctctgccaaaaattatggggacatcatgaagccccttgagcatctgacttctggcta
ataaaggaaatttattttcattgcaatagtgtgttggaattttttgtgtctctcactcggaaggacat
atgggagggcaaatcatttaaaacatcagaatgagtatttggtttagagtttggcaacatatgccata
tgctggctgccatgaacaaaggtggctataaagaggtcatcagtatatgaaacagccccctgctgtcc
attccttattccatagaaaagccttgacttgaggttagattttttttatattttgttttgtgttattt
ttttctttaacatccctaaaattttccttacatgttttactagccagatttttcctcctctcctgact
actcccagtcatagctgtccctcttctcttatgaagatccctcgacctgcagcccaagcttggcgtaa
tcatggtcatagctgtttcctgtgtgaaattgttatccgctcacaattccacacaacatacgagccgg
aagcataaagtgtaaagcctggggtgcctaatgagtgagctaactcacattaattgcgttgcgctcac
tgcccgctttccagtcgggaaacctgtcgtgccagcggatcgatccgctgcattaatgaatcggccaa
cgcgcggggagaggcggtttgcgtattgggcgctcttccgcttcctcgctcactgactcgctgcgctc
ggtcgttcggctgcggcgagcggtatcagctcactcaaaggcggtaatacggttatccacagaatcag
gggataacgcaggaaagaacatgtgagcaaaaggccagcaaaaggccaggaaccgtaaaaaggccgcg
ttgctggcgtttttccataggctccgcccccctgacgagcatcacaaaaatcgacgctcaagtcagag
gtggcgaaacccgacaggactataaagataccaggcgtttccccctggaagctccctcgtgcgctctc
ctgttccgaccctgccgcttaccggatacctgtccgcctttctcccttcgggaagcgtggcgctttct
caatgctcacgctgtaggtatctcagttcggtgtaggtcgttcgctccaagctgggctgtgtgcacga
accccccgttcagcccgaccgctgcgccttatccggtaactatcgtcttgagtccaacccggtaagac
acgacttatcgccactggcagcagccactggtaacaggattagcagagcgaggtatgtaggcggtgct
acagagttcttgaagtggtggcctaactacggctacactagaaggacagtatttggtatctgcgctct
gctgaagccagttaccttcggaaaaagagttggtagctcttgatccggcaaacaaaccaccgctggta
gcggtggtttttttgtttgcaagcagcagattacgcgcagaaaaaaaggatctcaagaagatcctttg
atcttttctacggggtctgacgctcagtggaacgaaaactcacgttaagggattttggtcatgagatt
atcaaaaaggatcttcacctagatccttttaaattaaaaatgaagttttaaatcaatctaaagtatat
atgagtaaacttggtctgacagttaccaatgcttaatcagtgaggcacctatctcagcgatctgtcta
tttcgttcatccatagttgcctgactccccgtcgtgtagataactacgatacgggagggcttaccatc
tggccccagtgctgcaatgataccgcgagacccacgctcaccggctccagatttatcagcaataaacc
agccagccggaagggccgagcgcagaagtggtcctgcaactttatccgcctccatccagtctattaat
tgttgccgggaagctagagtaagtagttcgccagttaatagtttgcgcaacgttgttgccattgctac
aggcatcgtggtgtcacgctcgtcgtttggtatggcttcattcagctccggttcccaacgatcaaggc
gagttacatgatcccccatgttgtgcaaaaaagcggttagctccttcggtcctccgatcgttgtcaga
agtaagttggccgcagtgttatcactcatggttatggcagcactgcataattctcttactgtcatgcc
atccgtaagatgcttttctgtgactggtgagtactcaaccaagtcattctgagaatagtgtatgcggc
gaccgagttgctcttgcccggcgtcaatacgggataataccgcgccacatagcagaactttaaaagtg
ctcatcattggaaaacgttcttcggggcgaaaactctcaaggatcttaccgctgttgagatccagttc
gatgtaacccactcgtgcacccaactgatcttcagcatcttttactttcaccagcgtttctgggtgag
caaaaacaggaaggcaaaatgccgcaaaaaagggaataagggcgacacggaaatgttgaatactcata
ctcttcctttttcaatattattgaagcatttatcagggttattgtctcatgagcggatacatatttga
atgtatttagaaaaataaacaaataggggttccgcgcacatttccccgaaaagtgccacctg
pJP61-AAV-EF1a-minigenes-v1-WPRE-codonoptimized
SEQ ID NO: 73
ctgcgcgctcgctcgctcactgaggccgcccgggcaaagcccgggcgtcgggcgacctttggtcgccc
ggcctcagtgagcgagcgagcgcgcagagagggagtggccaactccatcactaggggttccttgtagt
taatgattaacccgccatgctacttatctaccagggtaatggggatcctctagaactatagctagaat
tctagtggatccccccgcgccggctccggtgcccgtcagtgggcagagcgcacatcgcccacagtccc
cgagaagttggggggaggggtcggcaattgaaccggtgcctagagaaggtggcgcggggtaaactggg
aaagtgatgtcgtgtactggctccgcctttttcccgagggtgggggagaaccgtatataagtgcagta
gtcgccgtgaacgttctttttcgcaacgggtttgccgccagaacacaggtaagtgccgtgtgtggttc
ccgcgggcctggcctctttacgggttatggcccttgcgtgccttgaattacttccacctggctgcagt
acgtgattcttgatcccgagcttcgggttggaagtgggtgggagagttcgaggccttgcgcttaagga
gccccttcgcctcgtgcttgagttgaggcctggcctgggcgctggggccgccgcgtgcgaatctggtg
gcaccttcgcgcctgtctcgctgctttcgataagtctctagccatttaaaatttttgatgacctgctg
cgacgctttttttctggcaagatagtcttgtaaatgcgggccaagatctgcacactggtatttcggtt
tttggggccgcgggcggcgacggggcccgtgcgtcccagcgcacatgttcggcgaggcggggcctgcg
agcgcggccaccgagaatcggacgggggtagtctcaagctggccggcctgctctggtgcctggcctcg
cgccgccgtgtatcgccccgccctgggcggcaaggctggcccggtcggcaccagttgcgtgagcggaa
agatggccgcttcccggccctgctgcagggagctcaaaatggaggacgcggcgctcgggagagcgggc
gggtgagtcacccacacaaaggaaaagggcctttccgtcctcagccgtcgcttcatgtgactccacgg
agtaccgggcgccgtccaggcacctcgattagttctcgagcttttggagtacgtcgtctttaggttgg
ggggaggggttttatgcgatggagtttccccacactgagtgggtggagactgaagttaggccagcttg
gcacttgatgtaattctccttggaatttgccctttttgagtttggatcttggttcattctcaagcctc
agacagtggttcaaagtttttttcttccatttcaggtgtcgtgagctagagctttattgcggtagttt
atcacagttaaattgctaacgcagtcagtgcttctgacacaacagtctcgaacttaagctgcagaagt
tggtcgtgaggcactgggcaggtaagtatcaaggttacaagacaggtttaaggagaccaatagaaact
gggcttgtcgagacagagaagactcttgcgtttctgataggcacctattggtcttactgacatccact
ttgcctttctctccacaggtgtccactcccagttcaattacagctcttaaggctagagtacttaatac
gactcactataggctagcctcgagaattgtacaattcacgcgggccgccATGGTGAACTGTACTGAAG
TACCCGTTGCCATTCACGCAGATCAACTTACCCCTACCTGGCGCGTCTATTCCACAGGGAGTAATGTC
TTTCAGACCGCAGCATATAATGGGACAATAACCGTTGAGGAATTGAAAAAACTGTTGGAACAGGCTGC
ATACTATGTGTACTCTCGAGTCAAGAACCTGAACAGTAGCAGGGTCCCAGACCTGCTCGTCGCCGCAT
ACAACGGGCCTCAGAATCAGAGAAACGCCCCCAGAATAACCTTTGGGGGACCCTCCGATTCAACAGGC
AGCAATCAAAACGGAGAGCGGAGCGGCGCAAGGTCCAAACAGAGGCGGCCCCAAGGACTTCCTAACAA
TGCTGCCTACTATCAAACACAGACAAATTCACCTCGCCGGGCACGGTCAGTAGCTTCACAGTCTATTA
TAGCATACACTATGAGTTTGGGGGCTGAAAATAGTGTGGCCTACTCTAATAACAGTATCGCTGCTTAT
GAAGTACGGCAAATAGCTCCCGGACAAACAGGGAAGATTGCTGATTACAATTATAAGGCTGCCTATAA
ACTTGGAGCATCCCAACGGGTTGCCGGAGATTCAGGATTCGCAGCATATTCCAGATATAGAATCGGAA
ACTATAAAGCCGCCTACGACGCCTACAAAACCTTCCCACCCACCGAGCCAAAAAAGGACAAGAAAAAG
AAGGCTGATGAAACCCAGGCTTTGCCACAAAGGCAGAAGAAGCAGCAGACAGTAACCCTCCTCGCCGC
ATATGGAAAACAGGGCAATTTCAAAAATTTGAGGGAATTTGTCTTTAAGAATATAGACGGATATTTCA
AGATCTATTCTAAACATGCCGCCTACCACCACCATCATCACCATTAGtaagctcgcgtgatcaagctt
atcgataatcaacctctggattacaaaatttgtgaaagattgactggtattcttaactatgttgctcc
ttttacgctatgtggatacgctgctttaatgcctttgtatcatgctattgcttcccgtatggctttca
ttttctcctccttgtataaatcctggttgctgtctctttatgaggagttgtggcccgttgtcaggcaa
cgtggcgtggtgtgcactgtgtttgctgacgcaacccccactggttggggcattgccaccacctgtca
gctcctttccgggactttcgctttccccctccctattgccacggcggaactcatcgccgcctgccttg
cccgctgctggacaggggctcggctgttgggcactgacaattccgtggtgttgtcggggaaatcatcg
tcctttccttggctgctcgcctgtgttgccacctggattctgcgcgggacgtccttctgctacgtccc
ttcggccctcaatccagcggaccttccttcccgcggcctgctgccggctctgcggcctcttccgcgtc
ttcgccttcgccctcagacgagtcggatctccctttgggccgcctccccgcatcgataccgtcgaccc
gggcggcctcgaggacggggtgaactacgcctgaggatccgatctttttccctctgccaaaaattatg
gggacatcatgaagccccttgagcatctgacttctggctaataaaggaaatttattttcattgcaata
gtgtgttggaattttttgtgtctctcactcggaagcaattcgttgatctgaatttcgaccacccataa
tacccattaccctggtagataagtagcatggcgggttaatcattaactacaaggaacccctagtgatg
gagttggccactccctctctgcgcgctcgctcgctcactgaggccgggcgaccaaaggtcgcccgacg
cccgggctttgcccgggcggcctcagtgagcgagcgagcgcgcagccttaattaacctaattcactgg
ccgtcgttttacaacgtcgtgactgggaaaaccctggcgttacccaacttaatcgccttgcagcacat
ccccctttcgccagctggcgtaatagcgaagaggcccgcaccgatcgcccttcccaacagttgcgcag
cctgaatggcgaatgggacgcgccctgtagcggcgcattaagcgcggcgggtgtggtggttacgcgca
gcgtgaccgctacacttgccagcgccctagcgcccgctcctttcgctttcttcccttcctttctcgcc
acgttcgccggctttccccgtcaagctctaaatcgggggctccctttagggttccgatttagtgcttt
acggcacctcgaccccaaaaaacttgattagggtgatggttcacgtagtgggccatcgccctgataga
cggtttttcgccctttgacgttggagtccacgttctttaatagtggactcttgttccaaactggaaca
acactcaaccctatctcggtctattcttttgatttataagggattttgccgatttcggcctattggtt
aaaaaatgagctgatttaacaaaaatttaacgcgaattttaacaaaatattaacgcttacaatttagg
tggcacttttcggggaaatgtgcgcggaacccctatttgtttatttttctaaatacattcaaatatgt
atccgctcatgagacaataaccctgataaatgcttcaataatattgaaaaaggaagagtatgagtatt
caacatttccgtgtcgcccttattcccttttttgcggcattttgccttcctgtttttgctcacccaga
aacgctggtgaaagtaaaagatgctgaagatcagttgggtgcacgagtgggttacatcgaactggatc
tcaacagcggtaagatccttgagagttttcgccccgaagaacgttttccaatgatgagcacttttaaa
gttctgctatgtggcgcggtattatcccgtattgacgccgggcaagagcaactcggtcgccgcataca
ctattctcagaatgacttggttgagtactcaccagtcacagaaaagcatcttacggatggcatgacag
taagagaattatgcagtgctgccataaccatgagtgataacactgcggccaacttacttctgacaacg
atcggaggaccgaaggagctaaccgcttttttgcacaacatgggggatcatgtaactcgccttgatcg
ttgggaaccggagctgaatgaagccataccaaacgacgagcgtgacaccacgatgcctgtagcaatgg
caacaacgttgcgcaaactattaactggcgaactacttactctagcttcccggcaacaattaatagac
tggatggaggcggataaagttgcaggaccacttctgcgctcggcccttccggctggctggtttattgc
tgataaatctggagccggtgagcgtgggtctcgcggtatcattgcagcactggggccagatggtaagc
cctcccgtatcgtagttatctacacgacggggagtcaggcaactatggatgaacgaaatagacagatc
gctgagataggtgcctcactgattaagcattggtaactgtcagaccaagtttactcatatatacttta
gattgatttaaaacttcatttttaatttaaaaggatctaggtgaagatcctttttgataatctcatga
ccaaaatcccttaacgtgagttttcgttccactgagcgtcagaccccgtagaaaagatcaaaggatct
tcttgagatcctttttttctgcgcgtaatctgctgcttgcaaacaaaaaaaccaccgctaccagcggt
ggtttgtttgccggatcaagagctaccaactctttttccgaaggtaactggcttcagcagagcgcaga
taccaaatactgttcttctagtgtagccgtagttaggccaccacttcaagaactctgtagcaccgcct
acatacctcgctctgctaatcctgttaccagtggctgctgccagtggcgataagtcgtgtcttaccgg
gttggactcaagacgatagttaccggataaggcgcagcggtcgggctgaacggggggttcgtgcacac
agcccagcttggagcgaacgacctacaccgaactgagatacctacagcgtgagctatgagaaagcgcc
acgcttcccgaagggagaaaggcggacaggtatccggtaagcggcagggtcggaacaggagagcgcac
gagggagcttccagggggaaacgcctggtatctttatagtcctgtcgggtttcgccacctctgacttg
agcgtcgatttttgtgatgctcgtcaggggggcggagcctatggaaaaacgccagcaacgcggccttt
ttacggttcctggccttttgctggccttttgctcacatgttctttcctgcgttatcccctgattctgt
ggataaccgtattaccgcctttgagtgagctgataccgctcgccgcagccgaacgaccgagcgcagcg
agtcagtgagcgaggaagcggaagagcgcccaatacgcaaaccgcctctccccgcgcgttggccgatt
cattaatgcagctggcacgacaggtttcccgactggaaagcgggcagtgagcgcaacgcaattaatgt
gagttagctcactcattaggcaccccaggctttacactttatgcttccggctcgtatgttgtgtggaa
ttgtgagcggataacaatttcacacaggaaacagctatgaccatgattacgccagatttaattaaggc
cttaattagg
pJP62-AAV-EF1a-minigenes-v2-WPRE-codonoptimized
SEQ ID NO: 74
ctgcgcgctcgctcgctcactgaggccgcccgggcaaagcccgggcgtcgggcgacctttggtcgccc
ggcctcagtgagcgagcgagcgcgcagagagggagtggccaactccatcactaggggttccttgtagt
taatgattaacccgccatgctacttatctaccagggtaatggggatcctctagaactatagctagaat
tctagtggatccccccgcgccggctccggtgcccgtcagtgggcagagcgcacatcgcccacagtccc
cgagaagttggggggaggggtcggcaattgaaccggtgcctagagaaggtggcgcggggtaaactggg
aaagtgatgtcgtgtactggctccgcctttttcccgagggtgggggagaaccgtatataagtgcagta
gtcgccgtgaacgttctttttcgcaacgggtttgccgccagaacacaggtaagtgccgtgtgtggttc
ccgcgggcctggcctctttacgggttatggcccttgcgtgccttgaattacttccacctggctgcagt
acgtgattcttgatcccgagcttcgggttggaagtgggtgggagagttcgaggccttgcgcttaagga
gccccttcgcctcgtgcttgagttgaggcctggcctgggcgctggggccgccgcgtgcgaatctggtg
gcaccttcgcgcctgtctcgctgctttcgataagtctctagccatttaaaatttttgatgacctgctg
cgacgctttttttctggcaagatagtcttgtaaatgcgggccaagatctgcacactggtatttcggtt
tttggggccgcgggcggcgacggggcccgtgcgtcccagcgcacatgttcggcgaggcggggcctgcg
agcgcggccaccgagaatcggacgggggtagtctcaagctggccggcctgctctggtgcctggcctcg
cgccgccgtgtatcgccccgccctgggcggcaaggctggcccggtcggcaccagttgcgtgagcggaa
agatggccgcttcccggccctgctgcagggagctcaaaatggaggacgcggcgctcgggagagcgggc
gggtgagtcacccacacaaaggaaaagggcctttccgtcctcagccgtcgcttcatgtgactccacgg
agtaccgggcgccgtccaggcacctcgattagttctcgagcttttggagtacgtcgtctttaggttgg
ggggaggggttttatgcgatggagtttccccacactgagtgggtggagactgaagttaggccagcttg
gcacttgatgtaattctccttggaatttgccctttttgagtttggatcttggttcattctcaagcctc
agacagtggttcaaagtttttttcttccatttcaggtgtcgtgagctagagctttattgcggtagttt
atcacagttaaattgctaacgcagtcagtgcttctgacacaacagtctcgaacttaagctgcagaagt
tggtcgtgaggcactgggcaggtaagtatcaaggttacaagacaggtttaaggagaccaatagaaact
gggcttgtcgagacagagaagactcttgcgtttctgataggcacctattggtcttactgacatccact
ttgcctttctctccacaggtgtccactcccagttcaattacagctcttaaggctagagtacttaatac
gactcactataggctagcctcgagaattgtacaattcacgcgggccgccATGTTTGTTTTTCTCGTTT
TGCTCCCACTGGCAGCATACAACCTGATTGATTCCTATTTCGTAGTAGCAGCCTACCTTCTTCTGGAC
AGGCTCAATCAACTTGCTGCTTACAAATTGCTCGAACAATGGAACCTCGTCGCAGCCTATGTTCTGCT
TTTTCTTGCATTCGTAGTCGCTGCATATCTCCTGAGTGCAGGCATTTTCGGTGCTGCCGCTTATGTGT
TGGCCTGGCTCTATGCAGCCGTTGCTGCATACGCTTTGTGGGAAATCCAACAAGTCGTAGCTGCCTAC
ACCATGGCCGATCTCGTGTACGCCTTGGCCGCATACTTTCTGTGGTTGTTGTGGCCTGTGACAGCTGC
TTATTTTTTGGCATTTGTTGTGTTTTTGCTCGCAGCATATTTTCTTAAAAAGGACGCTCCCTACATAG
CTGCCTACTTCCTGAATAGGTTCACCACCACACTGGCTGCCTATTTGCTCATGCCCATTCTGACTTTG
ACAGCTGCATATAAAATAGCAGATTACAACTATAAGCTCGCTGCTTACTTCACAATCTCCGTAACCAC
TGAGATTGCCGCATATTTTGTGTTGGCTGCTGTTTATCGCATCGCAGCCTATTACCTGGCTACAGCAC
TGCTCACTCTTGCTGCATACCATCATCATCACCATCATTAGtaagctcgcgtgatcaagcttatcgat
aatcaacctctggattacaaaatttgtgaaagattgactggtattcttaactatgttgctccttttac
gctatgtggatacgctgctttaatgcctttgtatcatgctattgcttcccgtatggctttcattttct
cctccttgtataaatcctggttgctgtctctttatgaggagttgtggcccgttgtcaggcaacgtggc
gtggtgtgcactgtgtttgctgacgcaacccccactggttggggcattgccaccacctgtcagctcct
ttccgggactttcgctttccccctccctattgccacggcggaactcatcgccgcctgccttgcccgct
gctggacaggggctcggctgttgggcactgacaattccgtggtgttgtcggggaaatcatcgtccttt
ccttggctgctcgcctgtgttgccacctggattctgcgcgggacgtccttctgctacgtcccttcggc
cctcaatccagcggaccttccttcccgcggcctgctgccggctctgcggcctcttccgcgtcttcgcc
ttcgccctcagacgagtcggatctccctttgggccgcctccccgcatcgataccgtcgacccgggcgg
cctcgaggacggggtgaactacgcctgaggatccgatctttttccctctgccaaaaattatggggaca
tcatgaagccccttgagcatctgacttctggctaataaaggaaatttattttcattgcaatagtgtgt
tggaattttttgtgtctctcactcggaagcaattcgttgatctgaatttcgaccacccataataccca
ttaccctggtagataagtagcatggcgggttaatcattaactacaaggaacccctagtgatggagttg
gccactccctctctgcgcgctcgctcgctcactgaggccgggcgaccaaaggtcgcccgacgcccggg
ctttgcccgggcggcctcagtgagcgagcgagcgcgcagccttaattaacctaattcactggccgtcg
ttttacaacgtcgtgactgggaaaaccctggcgttacccaacttaatcgccttgcagcacatccccct
ttcgccagctggcgtaatagcgaagaggcccgcaccgatcgcccttcccaacagttgcgcagcctgaa
tggcgaatgggacgcgccctgtagcggcgcattaagcgcggcgggtgtggtggttacgcgcagcgtga
ccgctacacttgccagcgccctagcgcccgctcctttcgctttcttcccttcctttctcgccacgttc
gccggctttccccgtcaagctctaaatcgggggctccctttagggttccgatttagtgctttacggca
cctcgaccccaaaaaacttgattagggtgatggttcacgtagtgggccatcgccctgatagacggttt
ttcgccctttgacgttggagtccacgttctttaatagtggactcttgttccaaactggaacaacactc
aaccctatctcggtctattcttttgatttataagggattttgccgatttcggcctattggttaaaaaa
tgagctgatttaacaaaaatttaacgcgaattttaacaaaatattaacgcttacaatttaggtggcac
ttttcggggaaatgtgcgcggaacccctatttgtttatttttctaaatacattcaaatatgtatccgc
tcatgagacaataaccctgataaatgcttcaataatattgaaaaaggaagagtatgagtattcaacat
ttccgtgtcgcccttattcccttttttgcggcattttgccttcctgtttttgctcacccagaaacgct
ggtgaaagtaaaagatgctgaagatcagttgggtgcacgagtgggttacatcgaactggatctcaaca
gcggtaagatccttgagagttttcgccccgaagaacgttttccaatgatgagcacttttaaagttctg
ctatgtggcgcggtattatcccgtattgacgccgggcaagagcaactcggtcgccgcatacactattc
tcagaatgacttggttgagtactcaccagtcacagaaaagcatcttacggatggcatgacagtaagag
aattatgcagtgctgccataaccatgagtgataacactgcggccaacttacttctgacaacgatcgga
ggaccgaaggagctaaccgcttttttgcacaacatgggggatcatgtaactcgccttgatcgttggga
accggagctgaatgaagccataccaaacgacgagcgtgacaccacgatgcctgtagcaatggcaacaa
cgttgcgcaaactattaactggcgaactacttactctagcttcccggcaacaattaatagactggatg
gaggcggataaagttgcaggaccacttctgcgctcggcccttccggctggctggtttattgctgataa
atctggagccggtgagcgtgggtctcgcggtatcattgcagcactggggccagatggtaagccctccc
gtatcgtagttatctacacgacggggagtcaggcaactatggatgaacgaaatagacagatcgctgag
ataggtgcctcactgattaagcattggtaactgtcagaccaagtttactcatatatactttagattga
tttaaaacttcatttttaatttaaaaggatctaggtgaagatcctttttgataatctcatgaccaaaa
tcccttaacgtgagttttcgttccactgagcgtcagaccccgtagaaaagatcaaaggatcttcttga
gatcctttttttctgcgcgtaatctgctgcttgcaaacaaaaaaaccaccgctaccagcggtggtttg
tttgccggatcaagagctaccaactctttttccgaaggtaactggcttcagcagagcgcagataccaa
atactgttcttctagtgtagccgtagttaggccaccacttcaagaactctgtagcaccgcctacatac
ctcgctctgctaatcctgttaccagtggctgctgccagtggcgataagtcgtgtcttaccgggttgga
ctcaagacgatagttaccggataaggcgcagcggtcgggctgaacggggggttcgtgcacacagccca
gcttggagcgaacgacctacaccgaactgagatacctacagcgtgagctatgagaaagcgccacgctt
cccgaagggagaaaggcggacaggtatccggtaagcggcagggtcggaacaggagagcgcacgaggga
gcttccagggggaaacgcctggtatctttatagtcctgtcgggtttcgccacctctgacttgagcgtc
gatttttgtgatgctcgtcaggggggcggagcctatggaaaaacgccagcaacgcggcctttttacgg
ttcctggccttttgctggccttttgctcacatgttctttcctgcgttatcccctgattctgtggataa
ccgtattaccgcctttgagtgagctgataccgctcgccgcagccgaacgaccgagcgcagcgagtcag
tgagcgaggaagcggaagagcgcccaatacgcaaaccgcctctccccgcgcgttggccgattcattaa
tgcagctggcacgacaggtttcccgactggaaagcgggcagtgagcgcaacgcaattaatgtgagtta
gctcactcattaggcaccccaggctttacactttatgcttccggctcgtatgttgtgtggaattgtga
gcggataacaatttcacacaggaaacagctatgaccatgattacgccagatttaattaaggccttaat
tagg
pJP63-AAV-EF1a-minigenes-v3-WPRE-codonoptimized
SEQ ID NO: 75
ctgcgcgctcgctcgctcactgaggccgcccgggcaaagcccgggcgtcgggcgacctttggtcgccc
ggcctcagtgagcgagcgagcgcgcagagagggagtggccaactccatcactaggggttccttgtagt
taatgattaacccgccatgctacttatctaccagggtaatggggatcctctagaactatagctagaat
tctagtggatccccccgcgccggctccggtgcccgtcagtgggcagagcgcacatcgcccacagtccc
cgagaagttggggggaggggtcggcaattgaaccggtgcctagagaaggtggcgcggggtaaactggg
aaagtgatgtcgtgtactggctccgcctttttcccgagggtgggggagaaccgtatataagtgcagta
gtcgccgtgaacgttctttttcgcaacgggtttgccgccagaacacaggtaagtgccgtgtgtggttc
ccgcgggcctggcctctttacgggttatggcccttgcgtgccttgaattacttccacctggctgcagt
acgtgattcttgatcccgagcttcgggttggaagtgggtgggagagttcgaggccttgcgcttaagga
gccccttcgcctcgtgcttgagttgaggcctggcctgggcgctggggccgccgcgtgcgaatctggtg
gcaccttcgcgcctgtctcgctgctttcgataagtctctagccatttaaaatttttgatgacctgctg
cgacgctttttttctggcaagatagtcttgtaaatgcgggccaagatctgcacactggtatttcggtt
tttggggccgcgggcggcgacggggcccgtgcgtcccagcgcacatgttcggcgaggcggggcctgcg
agcgcggccaccgagaatcggacgggggtagtctcaagctggccggcctgctctggtgcctggcctcg
cgccgccgtgtatcgccccgccctgggcggcaaggctggcccggtcggcaccagttgcgtgagcggaa
agatggccgcttcccggccctgctgcagggagctcaaaatggaggacgcggcgctcgggagagcgggc
gggtgagtcacccacacaaaggaaaagggcctttccgtcctcagccgtcgcttcatgtgactccacgg
agtaccgggcgccgtccaggcacctcgattagttctcgagcttttggagtacgtcgtctttaggttgg
ggggaggggttttatgcgatggagtttccccacactgagtgggtggagactgaagttaggccagcttg
gcacttgatgtaattctccttggaatttgccctttttgagtttggatcttggttcattctcaagcctc
agacagtggttcaaagtttttttcttccatttcaggtgtcgtgagctagagctttattgcggtagttt
atcacagttaaattgctaacgcagtcagtgcttctgacacaacagtctcgaacttaagctgcagaagt
tggtcgtgaggcactgggcaggtaagtatcaaggttacaagacaggtttaaggagaccaatagaaact
gggcttgtcgagacagagaagactcttgcgtttctgataggcacctattggtcttactgacatccact
ttgcctttctctccacaggtgtccactcccagttcaattacagctcttaaggctagagtacttaatac
gactcactataggctagcctcgagaattgtacaattcacgcgggccgccATGACCCAAGATTTGTTTC
TGCCATTCTTTTCTAACGTCACCTGGTTTGCAGCTTACGACGGAGTTCCTTTCGTCGTTTCAACTGGC
TACCATTTCAGAGAAGCTGCATACGACGATCAAATCGGGTACTACAGGCGGGCAACTCGAAGGATAAG
AGCAGCTTATTGGATCTGCCTGCTTCAATTCGCTTACGCAAACAGAAATCGCTTTGCTGCTTATTCTG
TACTTTTGTTTCTTGCCTTCGTGGTGTTTCTCCTTGTTACAGCCGCTTATAATGAGTTTGCTTGTGTG
GTTGCTGACGCCGTGATCAAAACTCTGGCCGCCTATGATCTCCTCATAAGAAAATCTAACCATAACTT
TTTGGTTCAAGCCGCCGCATACGCAATCGCAAGTGAGTTCAGTAGTCTTCCATCCTACGCCGCTTTCG
CTGCCTACAAATATCTCTATTTTATAAAGGGATTGAACAATTTGAATCGCGGTGCCGCTTACCTGCTG
CTCCTTGACAGGCTTAATCAACTCGAATCCAAGATGTCTGCCGCTTACATGTGGCTCTCATACTTTAT
AGCCAGCTTCCGACTCTTCGCCAGGGCCGCTTATGAATTTGTATTTAAGAACATAGATGGGTACTTCA
AAATCTATAGCGCAGCCTACTTTCACCAAAAACTCCTTAAGTCCATTGCTGCCACTAGAGGCGCTGCT
GCTTACTTCCTTCTTGTGACCCTCGCCATTCTGACAGCTCTGAGGTTGTGTGCAGCTTACAGCGGTTA
CCTTAAATTGACAGACAATGTTTACATAAAAAACGCTGCCGCTTACCCCTTGAACATTATACCACTTA
CTACTGCTGCCAAGCTTATGGTCGCCGCTTACCATCACCACCACCATCATTAGtaagctcgcgtgatc
aagcttatcgataatcaacctctggattacaaaatttgtgaaagattgactggtattcttaactatgt
tgctccttttacgctatgtggatacgctgctttaatgcctttgtatcatgctattgcttcccgtatgg
ctttcattttctcctccttgtataaatcctggttgctgtctctttatgaggagttgtggcccgttgtc
aggcaacgtggcgtggtgtgcactgtgtttgctgacgcaacccccactggttggggcattgccaccac
ctgtcagctcctttccgggactttcgctttccccctccctattgccacggcggaactcatcgccgcct
gccttgcccgctgctggacaggggctcggctgttgggcactgacaattccgtggtgttgtcggggaaa
tcatcgtcctttccttggctgctcgcctgtgttgccacctggattctgcgcgggacgtccttctgcta
cgtcccttcggccctcaatccagcggaccttccttcccgcggcctgctgccggctctgcggcctcttc
cgcgtcttcgccttcgccctcagacgagtcggatctccctttgggccgcctccccgcatcgataccgt
cgacccgggcggcctcgaggacggggtgaactacgcctgaggatccgatctttttccctctgccaaaa
attatggggacatcatgaagccccttgagcatctgacttctggctaataaaggaaatttattttcatt
gcaatagtgtgttggaattttttgtgtctctcactcggaagcaattcgttgatctgaatttcgaccac
ccataatacccattaccctggtagataagtagcatggcgggttaatcattaactacaaggaaccccta
gtgatggagttggccactccctctctgcgcgctcgctcgctcactgaggccgggcgaccaaaggtcgc
ccgacgcccgggctttgcccgggcggcctcagtgagcgagcgagcgcgcagccttaattaacctaatt
cactggccgtcgttttacaacgtcgtgactgggaaaaccctggcgttacccaacttaatcgccttgca
gcacatccccctttcgccagctggcgtaatagcgaagaggcccgcaccgatcgcccttcccaacagtt
gcgcagcctgaatggcgaatgggacgcgccctgtagcggcgcattaagcgcggcgggtgtggtggtta
cgcgcagcgtgaccgctacacttgccagcgccctagcgcccgctcctttcgctttcttcccttccttt
ctcgccacgttcgccggctttccccgtcaagctctaaatcgggggctccctttagggttccgatttag
tgctttacggcacctcgaccccaaaaaacttgattagggtgatggttcacgtagtgggccatcgccct
gatagacggtttttcgccctttgacgttggagtccacgttctttaatagtggactcttgttccaaact
ggaacaacactcaaccctatctcggtctattcttttgatttataagggattttgccgatttcggccta
ttggttaaaaaatgagctgatttaacaaaaatttaacgcgaattttaacaaaatattaacgcttacaa
tttaggtggcacttttcggggaaatgtgcgcggaacccctatttgtttatttttctaaatacattcaa
atatgtatccgctcatgagacaataaccctgataaatgcttcaataatattgaaaaaggaagagtatg
agtattcaacatttccgtgtcgcccttattcccttttttgcggcattttgccttcctgtttttgctca
cccagaaacgctggtgaaagtaaaagatgctgaagatcagttgggtgcacgagtgggttacatcgaac
tggatctcaacagcggtaagatccttgagagttttcgccccgaagaacgttttccaatgatgagcact
tttaaagttctgctatgtggcgcggtattatcccgtattgacgccgggcaagagcaactcggtcgccg
catacactattctcagaatgacttggttgagtactcaccagtcacagaaaagcatcttacggatggca
tgacagtaagagaattatgcagtgctgccataaccatgagtgataacactgcggccaacttacttctg
acaacgatcggaggaccgaaggagctaaccgcttttttgcacaacatgggggatcatgtaactcgcct
tgatcgttgggaaccggagctgaatgaagccataccaaacgacgagcgtgacaccacgatgcctgtag
caatggcaacaacgttgcgcaaactattaactggcgaactacttactctagcttcccggcaacaatta
atagactggatggaggcggataaagttgcaggaccacttctgcgctcggcccttccggctggctggtt
tattgctgataaatctggagccggtgagcgtgggtctcgcggtatcattgcagcactggggccagatg
gtaagccctcccgtatcgtagttatctacacgacggggagtcaggcaactatggatgaacgaaataga
cagatcgctgagataggtgcctcactgattaagcattggtaactgtcagaccaagtttactcatatat
actttagattgatttaaaacttcatttttaatttaaaaggatctaggtgaagatcctttttgataatc
tcatgaccaaaatcccttaacgtgagttttcgttccactgagcgtcagaccccgtagaaaagatcaaa
ggatcttcttgagatcctttttttctgcgcgtaatctgctgcttgcaaacaaaaaaaccaccgctacc
agcggtggtttgtttgccggatcaagagctaccaactctttttccgaaggtaactggcttcagcagag
cgcagataccaaatactgttcttctagtgtagccgtagttaggccaccacttcaagaactctgtagca
ccgcctacatacctcgctctgctaatcctgttaccagtggctgctgccagtggcgataagtcgtgtct
taccgggttggactcaagacgatagttaccggataaggcgcagcggtcgggctgaacggggggttcgt
gcacacagcccagcttggagcgaacgacctacaccgaactgagatacctacagcgtgagctatgagaa
agcgccacgcttcccgaagggagaaaggcggacaggtatccggtaagcggcagggtcggaacaggaga
gcgcacgagggagcttccagggggaaacgcctggtatctttatagtcctgtcgggtttcgccacctct
gacttgagcgtcgatttttgtgatgctcgtcaggggggcggagcctatggaaaaacgccagcaacgcg
gcctttttacggttcctggccttttgctggccttttgctcacatgttctttcctgcgttatcccctga
ttctgtggataaccgtattaccgcctttgagtgagctgataccgctcgccgcagccgaacgaccgagc
gcagcgagtcagtgagcgaggaagcggaagagcgcccaatacgcaaaccgcctctccccgcgcgttgg
ccgattcattaatgcagctggcacgacaggtttcccgactggaaagcgggcagtgagcgcaacgcaat
taatgtgagttagctcactcattaggcaccccaggctttacactttatgcttccggctcgtatgttgt
gtggaattgtgagcggataacaatttcacacaggaaacagctatgaccatgattacgccagatttaat
taaggccttaattagg
pJP64-AAV-EF1a-minigenes-v4-WPRE-codonoptimized
SEQ ID NO: 76
ctgcgcgctcgctcgctcactgaggccgcccgggcaaagcccgggcgtcgggcgacctttggtcgccc
ggcctcagtgagcgagcgagcgcgcagagagggagtggccaactccatcactaggggttccttgtagt
taatgattaacccgccatgctacttatctaccagggtaatggggatcctctagaactatagctagaat
tctagtggatccccccgcgccggctccggtgcccgtcagtgggcagagcgcacatcgcccacagtccc
cgagaagttggggggaggggtcggcaattgaaccggtgcctagagaaggtggcgcggggtaaactggg
aaagtgatgtcgtgtactggctccgcctttttcccgagggtgggggagaaccgtatataagtgcagta
gtcgccgtgaacgttctttttcgcaacgggtttgccgccagaacacaggtaagtgccgtgtgtggttc
ccgcgggcctggcctctttacgggttatggcccttgcgtgccttgaattacttccacctggctgcagt
acgtgattcttgatcccgagcttcgggttggaagtgggtgggagagttcgaggccttgcgcttaagga
gccccttcgcctcgtgcttgagttgaggcctggcctgggcgctggggccgccgcgtgcgaatctggtg
gcaccttcgcgcctgtctcgctgctttcgataagtctctagccatttaaaatttttgatgacctgctg
cgacgctttttttctggcaagatagtcttgtaaatgcgggccaagatctgcacactggtatttcggtt
tttggggccgcgggcggcgacggggcccgtgcgtcccagcgcacatgttcggcgaggcggggcctgcg
agcgcggccaccgagaatcggacgggggtagtctcaagctggccggcctgctctggtgcctggcctcg
cgccgccgtgtatcgccccgccctgggcggcaaggctggcccggtcggcaccagttgcgtgagcggaa
agatggccgcttcccggccctgctgcagggagctcaaaatggaggacgcggcgctcgggagagcgggc
gggtgagtcacccacacaaaggaaaagggcctttccgtcctcagccgtcgcttcatgtgactccacgg
agtaccgggcgccgtccaggcacctcgattagttctcgagcttttggagtacgtcgtctttaggttgg
ggggaggggttttatgcgatggagtttccccacactgagtgggtggagactgaagttaggccagcttg
gcacttgatgtaattctccttggaatttgccctttttgagtttggatcttggttcattctcaagcctc
agacagtggttcaaagtttttttcttccatttcaggtgtcgtgagctagagctttattgcggtagttt
atcacagttaaattgctaacgcagtcagtgcttctgacacaacagtctcgaacttaagctgcagaagt
tggtcgtgaggcactgggcaggtaagtatcaaggttacaagacaggtttaaggagaccaatagaaact
gggcttgtcgagacagagaagactcttgcgtttctgataggcacctattggtcttactgacatccact
ttgcctttctctccacaggtgtccactcccagttcaattacagctcttaaggctagagtacttaatac
gactcactataggctagcctcgagaattgtacaattcacgcgggccgccATGACACCTATCAATCTCG
TGCGGGACCTTCCCCAAGGTTTCTCCGCTCTGGAGCCTTTGGTCGCTGCTTACTTGAACCAGCTTGAA
TCTAAGATGAGTGGGAAAGGCCAGCAACAACAGGGTCAAACCGTGACTAAGAAGTCTGCCGCTGAAGC
ATCCAAGAAGCCACGACAAAAGCGGACCGCCACCAAAGCATATAATGTGACACAGGCTTTTGGGCGAC
GAGGACCCGAGCAAACACAAGCCGCATACTATCTCCAGCCCAGGACTTTCCTGCTGGCCGCTTATCTT
CCTTTCTTTTCCAACGTAACTTGGTTCCACGCTATCCATGTTGCTGCCTATGATGCAGCTCTTGCCCT
CTTGCTCTTGGATCGCCTGAACCAGCTCGCCGCTTACTTCTTGTTCCTCACCTGGATCTGTCTCGCAG
CTTACAACAGAAATAGATTTCTCTACATAATCAAGCTCATATTTCTGTGGGCAGCTTATAGCGAGGAG
ACAGGAACTTTGATTGTAAACTCAGTGCTTCTTTTTGCCGCATATACCAACGGTACCAAACGCTTCGA
CAACCCTGTTCTGCCCTTCAACGCTGCATACCACGGGAAAGAGGATCTGAAATTTCCTAGAGGCCAGG
GCGTCCCCATAAATACCAACAGCTCCCCAGACGACCAAATTGGTTATTATCGCCGCGCTACCCGGAGG
ATCGCAGCCTATAGTGTCCTTTTGTTCCTGGCATTCGTGGCTGCATATAGATTCCCAAACATCACAAA
TTTGTGCCCTTTTGGCGAGGTCTTTAACGCTACCAGGTTTGCTGCTTACACACTCGCCTGCTTCGTAC
TGGCAGCCGTGTACCGGATCAACTGGGCAGCATACTTTTTGTTGGTTACTCTGGCAATTCTCGCTGCA
TACCATCACCATCACCACCATTAGtaagctcgcgtgatcaagcttatcgataatcaacctctggatta
caaaatttgtgaaagattgactggtattcttaactatgttgctccttttacgctatgtggatacgctg
ctttaatgcctttgtatcatgctattgcttcccgtatggctttcattttctcctccttgtataaatcc
tggttgctgtctctttatgaggagttgtggcccgttgtcaggcaacgtggcgtggtgtgcactgtgtt
tgctgacgcaacccccactggttggggcattgccaccacctgtcagctcctttccgggactttcgctt
tccccctccctattgccacggcggaactcatcgccgcctgccttgcccgctgctggacaggggctcgg
ctgttgggcactgacaattccgtggtgttgtcggggaaatcatcgtcctttccttggctgctcgcctg
tgttgccacctggattctgcgcgggacgtccttctgctacgtcccttcggccctcaatccagcggacc
ttccttcccgcggcctgctgccggctctgcggcctcttccgcgtcttcgccttcgccctcagacgagt
cggatctccctttgggccgcctccccgcatcgataccgtcgacccgggcggcctcgaggacggggtga
actacgcctgaggatccgatctttttccctctgccaaaaattatggggacatcatgaagccccttgag
catctgacttctggctaataaaggaaatttattttcattgcaatagtgtgttggaattttttgtgtct
ctcactcggaagcaattcgttgatctgaatttcgaccacccataatacccattaccctggtagataag
tagcatggcgggttaatcattaactacaaggaacccctagtgatggagttggccactccctctctgcg
cgctcgctcgctcactgaggccgggcgaccaaaggtcgcccgacgcccgggctttgcccgggcggcct
cagtgagcgagcgagcgcgcagccttaattaacctaattcactggccgtcgttttacaacgtcgtgac
tgggaaaaccctggcgttacccaacttaatcgccttgcagcacatccccctttcgccagctggcgtaa
tagcgaagaggcccgcaccgatcgcccttcccaacagttgcgcagcctgaatggcgaatgggacgcgc
cctgtagcggcgcattaagcgcggcgggtgtggtggttacgcgcagcgtgaccgctacacttgccagc
gccctagcgcccgctcctttcgctttcttcccttcctttctcgccacgttcgccggctttccccgtca
agctctaaatcgggggctccctttagggttccgatttagtgctttacggcacctcgaccccaaaaaac
ttgattagggtgatggttcacgtagtgggccatcgccctgatagacggtttttcgccctttgacgttg
gagtccacgttctttaatagtggactcttgttccaaactggaacaacactcaaccctatctcggtcta
ttcttttgatttataagggattttgccgatttcggcctattggttaaaaaatgagctgatttaacaaa
aatttaacgcgaattttaacaaaatattaacgcttacaatttaggtggcacttttcggggaaatgtgc
gcggaacccctatttgtttatttttctaaatacattcaaatatgtatccgctcatgagacaataaccc
tgataaatgcttcaataatattgaaaaaggaagagtatgagtattcaacatttccgtgtcgcccttat
tcccttttttgcggcattttgccttcctgtttttgctcacccagaaacgctggtgaaagtaaaagatg
ctgaagatcagttgggtgcacgagtgggttacatcgaactggatctcaacagcggtaagatccttgag
agttttcgccccgaagaacgttttccaatgatgagcacttttaaagttctgctatgtggcgcggtatt
atcccgtattgacgccgggcaagagcaactcggtcgccgcatacactattctcagaatgacttggttg
agtactcaccagtcacagaaaagcatcttacggatggcatgacagtaagagaattatgcagtgctgcc
ataaccatgagtgataacactgcggccaacttacttctgacaacgatcggaggaccgaaggagctaac
cgcttttttgcacaacatgggggatcatgtaactcgccttgatcgttgggaaccggagctgaatgaag
ccataccaaacgacgagcgtgacaccacgatgcctgtagcaatggcaacaacgttgcgcaaactatta
actggcgaactacttactctagcttcccggcaacaattaatagactggatggaggcggataaagttgc
aggaccacttctgcgctcggcccttccggctggctggtttattgctgataaatctggagccggtgagc
gtgggtctcgcggtatcattgcagcactggggccagatggtaagccctcccgtatcgtagttatctac
acgacggggagtcaggcaactatggatgaacgaaatagacagatcgctgagataggtgcctcactgat
taagcattggtaactgtcagaccaagtttactcatatatactttagattgatttaaaacttcattttt
aatttaaaaggatctaggtgaagatcctttttgataatctcatgaccaaaatcccttaacgtgagttt
tcgttccactgagcgtcagaccccgtagaaaagatcaaaggatcttcttgagatcctttttttctgcg
cgtaatctgctgcttgcaaacaaaaaaaccaccgctaccagcggtggtttgtttgccggatcaagagc
taccaactctttttccgaaggtaactggcttcagcagagcgcagataccaaatactgttcttctagtg
tagccgtagttaggccaccacttcaagaactctgtagcaccgcctacatacctcgctctgctaatcct
gttaccagtggctgctgccagtggcgataagtcgtgtcttaccgggttggactcaagacgatagttac
cggataaggcgcagcggtcgggctgaacggggggttcgtgcacacagcccagcttggagcgaacgacc
tacaccgaactgagatacctacagcgtgagctatgagaaagcgccacgcttcccgaagggagaaaggc
ggacaggtatccggtaagcggcagggtcggaacaggagagcgcacgagggagcttccagggggaaacg
cctggtatctttatagtcctgtcgggtttcgccacctctgacttgagcgtcgatttttgtgatgctcg
tcaggggggcggagcctatggaaaaacgccagcaacgcggcctttttacggttcctggccttttgctg
gccttttgctcacatgttctttcctgcgttatcccctgattctgtggataaccgtattaccgcctttg
agtgagctgataccgctcgccgcagccgaacgaccgagcgcagcgagtcagtgagcgaggaagcggaa
gagcgcccaatacgcaaaccgcctctccccgcgcgttggccgattcattaatgcagctggcacgacag
gtttcccgactggaaagcgggcagtgagcgcaacgcaattaatgtgagttagctcactcattaggcac
cccaggctttacactttatgcttccggctcgtatgttgtgtggaattgtgagcggataacaatttcac
acaggaaacagctatgaccatgattacgccagatttaattaaggccttaattagg
pJP65-AAV-EF1a-minigenes-v5-WPRE-codonoptimized
SEQ ID NO: 77
ctgcgcgctcgctcgctcactgaggccgcccgggcaaagcccgggcgtcgggcgacctttggtcgccc
ggcctcagtgagcgagcgagcgcgcagagagggagtggccaactccatcactaggggttccttgtagt
taatgattaacccgccatgctacttatctaccagggtaatggggatcctctagaactatagctagaat
tctagtggatccccccgcgccggctccggtgcccgtcagtgggcagagcgcacatcgcccacagtccc
cgagaagttggggggaggggtcggcaattgaaccggtgcctagagaaggtggcgcggggtaaactggg
aaagtgatgtcgtgtactggctccgcctttttcccgagggtgggggagaaccgtatataagtgcagta
gtcgccgtgaacgttctttttcgcaacgggtttgccgccagaacacaggtaagtgccgtgtgtggttc
ccgcgggcctggcctctttacgggttatggcccttgcgtgccttgaattacttccacctggctgcagt
acgtgattcttgatcccgagcttcgggttggaagtgggtgggagagttcgaggccttgcgcttaagga
gccccttcgcctcgtgcttgagttgaggcctggcctgggcgctggggccgccgcgtgcgaatctggtg
gcaccttcgcgcctgtctcgctgctttcgataagtctctagccatttaaaatttttgatgacctgctg
cgacgctttttttctggcaagatagtcttgtaaatgcgggccaagatctgcacactggtatttcggtt
tttggggccgcgggcggcgacggggcccgtgcgtcccagcgcacatgttcggcgaggcggggcctgcg
agcgcggccaccgagaatcggacgggggtagtctcaagctggccggcctgctctggtgcctggcctcg
cgccgccgtgtatcgccccgccctgggcggcaaggctggcccggtcggcaccagttgcgtgagcggaa
agatggccgcttcccggccctgctgcagggagctcaaaatggaggacgcggcgctcgggagagcgggc
gggtgagtcacccacacaaaggaaaagggcctttccgtcctcagccgtcgcttcatgtgactccacgg
agtaccgggcgccgtccaggcacctcgattagttctcgagcttttggagtacgtcgtctttaggttgg
ggggaggggttttatgcgatggagtttccccacactgagtgggtggagactgaagttaggccagcttg
gcacttgatgtaattctccttggaatttgccctttttgagtttggatcttggttcattctcaagcctc
agacagtggttcaaagtttttttcttccatttcaggtgtcgtgagctagagctttattgcggtagttt
atcacagttaaattgctaacgcagtcagtgcttctgacacaacagtctcgaacttaagctgcagaagt
tggtcgtgaggcactgggcaggtaagtatcaaggttacaagacaggtttaaggagaccaatagaaact
gggcttgtcgagacagagaagactcttgcgtttctgataggcacctattggtcttactgacatccact
ttgcctttctctccacaggtgtccactcccagttcaattacagctcttaaggctagagtacttaatac
gactcactataggctagcctcgagaattgtacaattcacgcgggccgccATGTGTAATGACCCTTTCC
TGGGGGTTTATTATCATAAGAATAATAAATCCTGGATGGAAAGCGAGTTCAGGGTCGCAGCATATGAT
CTGGATGACTTCTCTAAACAGCTCCAGCAGTCCATGTCATCTGCCGATAGCACTGCTGCATATAGCCT
GCTGATCGTAAATAACGCAACAAATGTCGTTATAAAAGTTGCTGCTTATAACTTGTTGAAGGACTGTC
CAGCCGTCGCCGCCTACTTTCACCAAAAGCTTCTTAAATCAATAGCAGCAACACGGGGAGCCGCTGCA
TATGTCGTCCAAACCATTGAGGTAAACAGTTTCTCTGGGTACCTGAAGGCCGCATACTATAAATTTGT
ACGCATTCAACCTGGGCAGACATTTTCAGTGCTGGCTGCATACGCAAAACTTATGGTCGTAATCCCAG
ACTACAATACTTACAAGAATGCAGCATACGTCTTGAAACTTAAAGTGGATACTGCTAATCCTAAAACA
CCAAAGGCTGCTTACACCGGAATCGCAGTAGAGCAGGATAAAAATACCCAGGAGGTATTCGCACAGGT
CAAGCAAATCTATAAAACTCCCCCTATTAAAGACTTTGGTGGTTTTAACTTTTCCCAGATTCTTCCCG
ATCCATCTAAACCAAGCAAAAGGAGCTTTATTGAAGATTTGCTGTTCAACAAAGTCACATTGGCTGAC
GCAGGTTTCATTAAACAGTATGGGGATTGCCTTGCCGCCTACAGAGGCATGGTTCTGGGGAGTCTCGC
AGCAACCGTCCGGCTTCAAGCCGCCTATGCTTTGGCATATTACAACACTACCAAGGCCGCATACCCAA
CAGTAGTGGTCAATGCAGCCAATGTTTATCTCAAGCACGGGGCTGCTTACCACCATCACCACCATCAT
TAGtaagctcgcgtgatcaagcttatcgataatcaacctctggattacaaaatttgtgaaagattgac
tggtattcttaactatgttgctccttttacgctatgtggatacgctgctttaatgcctttgtatcatg
ctattgcttcccgtatggctttcattttctcctccttgtataaatcctggttgctgtctctttatgag
gagttgtggcccgttgtcaggcaacgtggcgtggtgtgcactgtgtttgctgacgcaacccccactgg
ttggggcattgccaccacctgtcagctcctttccgggactttcgctttccccctccctattgccacgg
cggaactcatcgccgcctgccttgcccgctgctggacaggggctcggctgttgggcactgacaattcc
gtggtgttgtcggggaaatcatcgtcctttccttggctgctcgcctgtgttgccacctggattctgcg
cgggacgtccttctgctacgtcccttcggccctcaatccagcggaccttccttcccgcggcctgctgc
cggctctgcggcctcttccgcgtcttcgccttcgccctcagacgagtcggatctccctttgggccgcc
tccccgcatcgataccgtcgacccgggcggcctcgaggacggggtgaactacgcctgaggatccgatc
tttttccctctgccaaaaattatggggacatcatgaagccccttgagcatctgacttctggctaataa
aggaaatttattttcattgcaatagtgtgttggaattttttgtgtctctcactcggaagcaattcgtt
gatctgaatttcgaccacccataatacccattaccctggtagataagtagcatggcgggttaatcatt
aactacaaggaacccctagtgatggagttggccactccctctctgcgcgctcgctcgctcactgaggc
cgggcgaccaaaggtcgcccgacgcccgggctttgcccgggcggcctcagtgagcgagcgagcgcgca
gccttaattaacctaattcactggccgtcgttttacaacgtcgtgactgggaaaaccctggcgttacc
caacttaatcgccttgcagcacatccccctttcgccagctggcgtaatagcgaagaggcccgcaccga
tcgcccttcccaacagttgcgcagcctgaatggcgaatgggacgcgccctgtagcggcgcattaagcg
cggcgggtgtggtggttacgcgcagcgtgaccgctacacttgccagcgccctagcgcccgctcctttc
gctttcttcccttcctttctcgccacgttcgccggctttccccgtcaagctctaaatcgggggctccc
tttagggttccgatttagtgctttacggcacctcgaccccaaaaaacttgattagggtgatggttcac
gtagtgggccatcgccctgatagacggtttttcgccctttgacgttggagtccacgttctttaatagt
ggactcttgttccaaactggaacaacactcaaccctatctcggtctattcttttgatttataagggat
tttgccgatttcggcctattggttaaaaaatgagctgatttaacaaaaatttaacgcgaattttaaca
aaatattaacgcttacaatttaggtggcacttttcggggaaatgtgcgcggaacccctatttgtttat
ttttctaaatacattcaaatatgtatccgctcatgagacaataaccctgataaatgcttcaataatat
tgaaaaaggaagagtatgagtattcaacatttccgtgtcgcccttattcccttttttgcggcattttg
ccttcctgtttttgctcacccagaaacgctggtgaaagtaaaagatgctgaagatcagttgggtgcac
gagtgggttacatcgaactggatctcaacagcggtaagatccttgagagttttcgccccgaagaacgt
tttccaatgatgagcacttttaaagttctgctatgtggcgcggtattatcccgtattgacgccgggca
agagcaactcggtcgccgcatacactattctcagaatgacttggttgagtactcaccagtcacagaaa
agcatcttacggatggcatgacagtaagagaattatgcagtgctgccataaccatgagtgataacact
gcggccaacttacttctgacaacgatcggaggaccgaaggagctaaccgcttttttgcacaacatggg
ggatcatgtaactcgccttgatcgttgggaaccggagctgaatgaagccataccaaacgacgagcgtg
acaccacgatgcctgtagcaatggcaacaacgttgcgcaaactattaactggcgaactacttactcta
gcttcccggcaacaattaatagactggatggaggcggataaagttgcaggaccacttctgcgctcggc
ccttccggctggctggtttattgctgataaatctggagccggtgagcgtgggtctcgcggtatcattg
cagcactggggccagatggtaagccctcccgtatcgtagttatctacacgacggggagtcaggcaact
atggatgaacgaaatagacagatcgctgagataggtgcctcactgattaagcattggtaactgtcaga
ccaagtttactcatatatactttagattgatttaaaacttcatttttaatttaaaaggatctaggtga
agatcctttttgataatctcatgaccaaaatcccttaacgtgagttttcgttccactgagcgtcagac
cccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaagg
taactggcttcagcagagcgcagataccaaatactgttcttctagtgtagccgtagttaggccaccac
ttcaagaactctgtagcaccgcctacatacctcgctctgctaatcctgttaccagtggctgctgccag
tggcgataagtcgtgtcttaccgggttggactcaagacgatagttaccggataaggcgcagcggtcgg
gctgaacggggggttcgtgcacacagcccagcttggagcgaacgacctacaccgaactgagataccta
cagcgtgagctatgagaaagcgccacgcttcccgaagggagaaaggcggacaggtatccggtaagcgg
cagggtcggaacaggagagcgcacgagggagcttccagggggaaacgcctggtatctttatagtcctg
tcgggtttcgccacctctgacttgagcgtcgatttttgtgatgctcgtcaggggggcggagcctatgg
aaaaacgccagcaacgcggcctttttacggttcctggccttttgctggccttttgctcacatgttctt
tcctgcgttatcccctgattctgtggataaccgtattaccgcctttgagtgagctgataccgctcgcc
gcagccgaacgaccgagcgcagcgagtcagtgagcgaggaagcggaagagcgcccaatacgcaaaccg
cctctccccgcgcgttggccgattcattaatgcagctggcacgacaggtttcccgactggaaagcggg
cagtgagcgcaacgcaattaatgtgagttagctcactcattaggcaccccaggctttacactttatgc
ttccggctcgtatgttgtgtggaattgtgagcggataacaatttcacacaggaaacagctatgaccat
gattacgccagatttaattaaggccttaattagg
pVP22b-B.1.351-hextra Pro-UTR
SEQ ID NO: 78
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATgTTTGTGTTCCTGGTGCTGCTGCCACTGGTGTCCAGCCAGTGTGTGAACTTCACCACCAG
GACCCAACTTCCTCCTGCCTACACCAACTCCTTCACCAGGGGAGTCTACTACCCTGACAAGGTGTTCA
GGTCCTCTGTGCTGCACAGCACCCAGGACCTGTTCCTGCCATTCTTCAGCAATGTGACCTGGTTCCAT
GCCATCCATGTGTCTGGCACCAATGGCACCAAGAGGTTTGCCAACCCTGTGCTGCCATTCAATGATGG
AGTCTACTTTGCCAGCACAGAGAAGAGCAACATCATCAGGGGCTGGATTTTTGGCACCACCCTGGACA
GCAAGACCCAGTCCCTGCTGATTGTGAACAATGCCACCAATGTGGTGATTAAGGTGTGTGAGTTCCAG
TTCTGTAATGACCCATTCCTGGGAGTCTACTACCACAAGAACAACAAGTCCTGGATGGAGTCTGAGTT
CAGGGTCTACTCCTCTGCCAACAACTGTACCTTTGAATATGTGAGCCAACCATTCCTGATGGACTTGG
AGGGCAAGCAGGGCAACTTCAAGAACCTGAGGGAGTTTGTGTTCAAGAACATTGATGGCTACTTCAAG
ATTTACAGCAAACACACACCAATCAACCTGGTGAGGGGCCTGCCACAGGGCTTCTCTGCCTTGGAACC
ACTGGTGGACCTGCCAATTGGCATCAACATCACCAGGTTCCAGACCCTGCTGGCTCTGCACATTTCCT
ACCTGACACCTGGAGACTCCTCCTCTGGCTGGACAGCAGGAGCAGCAGCCTACTATGTGGGCTACCTC
CAACCAAGGACCTTCCTGCTGAAATACAATGAGAATGGCACCATCACAGATGCTGTGGACTGTGCCCT
GGACCCACTGTCTGAGACCAAGTGTACCCTGAAATCCTTCACAGTGGAGAAGGGCATCTACCAGACCA
GCAACTTCAGGGTCCAACCAACAGAGAGCATTGTGAGGTTTCCAAACATCACCAACCTGTGTCCATTT
GGAGAGGTGTTCAATGCCACCAGGTTTGCCTCTGTCTATGCCTGGAACAGGAAGAGGATTAGCAACTG
TGTGGCTGACTACTCTGTGCTCTACAACTCTGCCTCCTTCAGCACCTTCAAGTGTTATGGAGTGAGCC
CAACCAAACTGAATGACCTGTGTTTCACCAATGTCTATGCTGACTCCTTTGTGATTAGGGGAGATGAG
GTGAGACAGATTGCCCCTGGACAAACAGGCAACATTGCTGACTACAACTACAAACTGCCTGATGACTT
CACAGGCTGTGTGATTGCCTGGAACAGCAACAACCTGGACAGCAAGGTGGGAGGCAACTACAACTACC
TCTACAGACTGTTCAGGAAGAGCAACCTGAAACCATTTGAGAGGGACATCAGCACAGAGATTTACCAG
GCTGGCAGCACACCATGTAATGGAGTGAAGGGCTTCAACTGTTACTTTCCACTCCAATCCTATGGCTT
CCAACCAACCTACGGAGTGGGCTACCAACCATACAGGGTGGTGGTGCTGTCCTTTGAACTGCTCCATG
CCCCTGCCACAGTGTGTGGACCAAAGAAGAGCACCAACCTGGTGAAGAACAAGTGTGTGAACTTCAAC
TTCAATGGACTGACAGGCACAGGAGTGCTGACAGAGAGCAACAAGAAGTTCCTGCCATTCCAACAGTT
TGGCAGGGACATTGCTGACACCACAGATGCTGTGAGGGACCCACAGACCTTGGAGATTCTGGACATCA
CACCATGTTCCTTTGGAGGAGTGTCTGTGATTACACCTGGCACCAACACCAGCAACCAGGTGGCTGTG
CTCTACCAGGATGTGAACTGTACTGAGGTGCCTGTGGCTATCCATGCTGACCAACTTACACCAACCTG
GAGGGTCTACAGCACAGGCAGCAATGTGTTCCAGACCAGGGCTGGCTGTCTGATTGGAGCAGAGCATG
TGAACAACTCCTATGAGTGTGACATCCCAATTGGAGCAGGCATCTGTGCCTCCTACCAGACCCAGACC
AACAGCCCAGGCTCTGCATCTTCTGTGGCAAGCCAGAGCATCATTGCCTACACAATGAGTCTGGGAGT
GGAGAACTCTGTGGCTTACAGCAACAACAGCATTGCCATCCCAACCAACTTCACCATCTCTGTGACCA
CAGAGATTCTGCCTGTGAGTATGACCAAGACCTCTGTGGACTGTACAATGTATATCTGTGGAGACAGC
ACAGAGTGTAGCAACCTGCTGCTCCAATATGGCTCCTTCTGTACCCAACTTAACAGGGCTCTGACAGG
CATTGCTGTGGAACAGGACAAGAACACCCAGGAGGTGTTTGCCCAGGTGAAGCAGATTTACAAGACAC
CTCCAATCAAGGACTTTGGAGGCTTCAACTTCAGCCAGATTCTGCCTGACCCAAGCAAGCCAAGCAAG
AGGTCCCCTATTGAGGACCTGCTGTTCAACAAGGTGACCCTGGCTGATGCTGGCTTCATCAAGCAATA
TGGAGACTGTCTGGGAGACATCGCTGCTAGGGACCTGATTTGTGCCCAGAAGTTCAATGGACTGACAG
TGCTGCCTCCACTGCTGACAGATGAGATGATTGCCCAATACACCTCTGCCCTGCTGGCTGGAACCATC
ACCTCTGGATGGACCTTTGGAGCAGGACCAGCCCTCCAAATCCCATTTCCAATGCAGATGGCTTACAG
GTTCAATGGCATTGGAGTGACCCAGAATGTGCTCTATGAGAACCAGAAACTGATTGCCAACCAGTTCA
ACTCTGCCATTGGCAAGATTCAGGACTCCCTGTCCAGCACACCATCTGCCCTGGGCAAGCTCCAAGAT
GTGGTGAACCAGAATGCCCAGGCTCTGAACACCCTGGTGAAGCAACTTTCCAGCAACTTTGGAGCCAT
CTCCTCTGTGCTGAATGACATCCTGAGCAGACTGGACCCACCAGAGGCTGAGGTCCAGATTGACAGAC
TGATTACAGGCAGACTCCAATCCCTCCAAACCTATGTGACCCAACAACTTATCAGGGCTGCTGAGATT
AGGGCATCTGCCAACCTGGCTGCCACCAAGATGAGTGAGTGTGTGCTGGGACAAAGCAAGAGGGTGGA
CTTCTGTGGCAAGGGCTACCACCTGATGAGTTTTCCACAGTCTGCCCCTCATGGAGTGGTGTTCCTGC
ATGTGACCTATGTGCCTGCCCAGGAGAAGAACTTCACCACAGCCCCTGCCATCTGCCATGATGGCAAG
GCTCACTTTCCAAGGGAGGGAGTGTTTGTGAGCAATGGCACCCACTGGTTTGTGACCCAGAGGAACTT
CTATGAACCACAGATTATCACCACAGACAACACCTTTGTGTCTGGCAACTGTGATGTGGTGATTGGCA
TTGTGAACAACACAGTCTATGACCCACTCCAACCTGAACTGGACTCCTTCAAGGAGGAACTGGACAAA
TACTTCAAGAACCACACCAGCCCTGATGTGGACCTGGGAGACATCTCTGGCATCAATGCCTCTGTGGT
GAACATCCAGAAGGAGATTGACAGACTGAATGAGGTGGCTAAGAACCTGAATGAGTCCCTGATTGACC
TCCAAGAACTGGGCAAATATGAACAATACATCAAGTGGCCATGGTACATCTGGCTGGGCTTCATTGCT
GGACTGATTGCCATTGTGATGGTGACCATAATGCTGTGTTGTATGACCTCCTGTTGTTCCTGTCTGAA
AGGCTGTTGTTCCTGTGGCTCCTGTTGTAAGTTTGATGAGGATGACTCTGAACCTGTGCTGAAAGGAG
TGAAACTGCACTACACCTGATGACTCGAGCTGGTACTGCATGCACGCAATGCTAGCTGCCCCTTTCCC
GTCCTGGGTACCCCGAGTCTCCCCCGACCTCGGGTCCCAGGTATGCTCCCACCTCCACCTGCCCCACT
CACCACCTCTGCTAGTTCCAGACACCTCCCAAGCACGCAGCAATGCAGCTCAAAACGCTTAGCCTAGC
CACACCCCCACGGGAAACAGCAGTGATTAACCTTTAGCAATAAACGAAAGTTTAACTAAGCTATACTA
ACCCCAGGGTTGGTCAATTTCGTGCCAGCCACACCCTGGAGCTAGCaaaccgctgatcagcctcgact
gtgccttctagttgccagccatctgttgtttgcccctcccccgtgccttccttgaccctggaaggtgc
cactcccactgtcctttcctaataaaatgaggaaattgcatcgcattgtctgagtaggtgtcattcta
ttctggggggtggggtggggcaggacagcaagggggaggattgggaagacaatagcaggcatgctggg
gatgcggtgggctctatggcttctgaggcggaaagaaccagctggggctctagggggtatccccacgc
gccctgtagcggcgcattaagcgcggcgggtgtggtggttacgcgcagcgtgaccgctacacttgcca
gcgccctagcgcccgctcctttcgctttcttcccttcctttctcgccacgttcgccggctttccccgt
caagctctaaatcgggggctccctttagggttccgatttagtgctttacggcacctcgaccccaaaaa
acttgattagggtgatggttcacgtagtgggccatcgccctgatagacggtttttcgccctttgacgt
tggagtccacgttctttaatagtggactcttgttccaaactggaacaacactcaaccctatctcggtc
tattcttttgatttataagggattttgccgatttcggcctattggttaaaaaatgagctgatttaaca
aaaatttaacgcgaattaattctgtggaatgtgtgtcagttagggtgtggaaagtccccaggctcccc
agcaggcagaagtatgcaaagcatgcatctcaattagtcagcaaccaggtgtggaaagtccccaggct
ccccagcaggcagaagtatgcaaagcatgcatctcaattagtcagcaaccatagtcccgcccctaact
ccgcccatcccgcccctaactccgcccagttccgcccattctccgccccatggctgactaattttttt
tatttatgcagaggccgaggccgcctctgcctctgagctattccagaagtagtgaggaggcttttttg
gaggcctaggcttttgcaaaaagctcccgggagcttgtatatccattttcggatctgatcaagagaca
ggatgaggatcgtttcgcatgattgaacaagatggattgcacgcaggttctccggccgcttgggtgga
gaggctattcggctatgactgggcacaacagacaatcggctgctctgatgccgccgtgttccggctgt
cagcgcaggggcgcccggttctttttgtcaagaccgacctgtccggtgccctgaatgaactgcaggac
gaggcagcgcggctatcgtggctggccacgacgggcgttccttgcgcagctgtgctcgacgttgtcac
tgaagcgggaagggactggctgctattgggcgaagtgccggggcaggatctcctgtcatctcaccttg
ctcctgccgagaaagtatccatcatggctgatgcaatgcggcggctgcatacgcttgatccggctacc
tgcccattcgaccaccaagcgaaacatcgcatcgagcgagcacgtactcggatggaagccggtcttgt
cgatcaggatgatctggacgaagagcatcaggggctcgcgccagccgaactgttcgccaggctcaagg
cgcgcatgcccgacggcgaggatctcgtcgtgacccatggcgatgcctgcttgccgaatatcatggtg
gaaaatggccgcttttctggattcatcgactgtggccggctgggtgtggcggaccgctatcaggacat
agcgttggctacccgtgatattgctgaagagcttggcggcgaatgggctgaccgcttcctcgtgcttt
acggtatcgccgctcccgattcgcagcgcatcgccttctatcgccttcttgacgagttcttctgagcg
ggactctggggttcgaaatgaccgaccaagcgacgcccaacctgccatcacgagatttcgattccacc
gccgccttctatgaaaggttgggcttcggaatcgttttccgggacgccggctggatgatcctccagcg
cggggatctcatgctggagttcttcgcccaccccaacttgtttattgcagcttataatggttacaaat
aaagcaatagcatcacaaatttcacaaataaagcatttttttcactgcattctagttgtggtttgtcc
aaactcatcaatgtatcttatcatgtctgtataccgtcgacctctagctagagcttggcgtaatcatg
gtcatagctgtttcctgtgtgaaattgttatccgctcacaattccacacaacatacgagccggaagca
taaagtgtaaagcctggggtgcctaatgagtgagctaactcacattaattgcgttgcgctcactgccc
gctttccagtcgggaaacctgtcgtgccagctgcattaatgaatcggccaacgcgcggggagaggcgg
tttgcgtattgggcgctcttccgcttcctcgctcactgactcgctgcgctcggtcgttcggctgcggc
gagcggtatcagctcactcaaaggcggtaatacggttatccacagaatcaggggataacgcaggaaag
aacatgtgagcaaaaggccagcaaaaggccaggaaccgtaaaaaggccgcgttgctggcgtttttcca
taggctccgcccccctgacgagcatcacaaaaatcgacgctcaagtcagaggtggcgaaacccgacag
gactataaagataccaggcgtttccccctggaagctccctcgtgcgctctcctgttccgaccctgccg
cttaccggatacctgtccgcctttctcccttcgggaagcgtggcgctttctcatagctcacgctgtag
gtatctcagttcggtgtaggtcgttcgctccaagctgggctgtgtgcacgaaccccccgttcagcccg
accgctgcgccttatccggtaactatcgtcttgagtccaacccggtaagacacgacttatcgccactg
gcagcagccactggtaacaggattagcagagcgaggtatgtaggcggtgctacagagttcttgaagtg
gtggcctaactacggctacactagaagaacagtatttggtatctgcgctctgctgaagccagttacct
tcggaaaaagagttggtagctcttgatccggcaaacaaaccaccgctggtagcggtggtttttttgtt
tgcaagcagcagattacgcgcagaaaaaaaggatctcaagaagatcctttgatcttttctacggggtc
tgacgctcagtggaacgaaaactcacgttaagggattttggtcatgagattatcaaaaaggatcttca
cctagatccttttaaattaaaaatgaagttttaaatcaatctaaagtatatatgagtaaacttggtct
gacagttaccaatgcttaatcagtgaggcacctatctcagcgatctgtctatttcgttcatccatagt
tgcctgactccccgtcgtgtagataactacgatacgggagggcttaccatctggccccagtgctgcaa
tgataccgcgagacccacgctcaccggctccagatttatcagcaataaaccagccagccggaagggcc
gagcgcagaagtggtcctgcaactttatccgcctccatccagtctattaattgttgccgggaagctag
agtaagtagttcgccagttaatagtttgcgcaacgttgttgccattgctacaggcatcgtggtgtcac
gctcgtcgtttggtatggcttcattcagctccggttcccaacgatcaaggcgagttacatgatccccc
atgttgtgcaaaaaagcggttagctccttcggtcctccgatcgttgtcagaagtaagttggccgcagt
gttatcactcatggttatggcagcactgcataattctcttactgtcatgccatccgtaagatgctttt
ctgtgactggtgagtactcaaccaagtcattctgagaatagtgtatgcggcgaccgagttgctcttgc
ccggcgtcaatacgggataataccgcgccacatagcagaactttaaaagtgctcatcattggaaaacg
ttcttcggggcgaaaactctcaaggatcttaccgctgttgagatccagttcgatgtaacccactcgtg
cacccaactgatcttcagcatcttttactttcaccagcgtttctgggtgagcaaaaacaggaaggcaa
aatgccgcaaaaaagggaataagggcgacacggaaatgttgaatactcatactcttcctttttcaata
ttattgaagcatttatcagggttattgtctcatgagcggatacatatttgaatgtatttagaaaaata
aacaaataggggttccgcgcacatttccccgaaaagtgccacctgacgtc
pVP27-mRNA UTR basic
SEQ ID NO: 79
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATggatccgagctcggtaccaagcttaagtttCTCGAGCTGGTACTGCATGCACGCAATGCT
AGCTGCCCCTTTCCCGTCCTGGGTACCCCGAGTCTCCCCCGACCTCGGGTCCCAGGTATGCTCCCACC
TCCACCTGCCCCACTCACCACCTCTGCTAGTTCCAGACACCTCCCAAGCACGCAGCAATGCAGCTCAA
AACGCTTAGCCTAGCCACACCCCCACGGGAAACAGCAGTGATTAACCTTTAGCAATAAACGAAAGTTT
AACTAAGCTATACTAACCCCAGGGTTGGTCAATTTCGTGCCAGCCACACCCTGGAGCTAGCaaaccgc
tgatcagcctcgactgtgccttctagttgccagccatctgttgtttgcccctcccccgtgccttcctt
gaccctggaaggtgccactcccactgtcctttcctaataaaatgaggaaattgcatcgcattgtctga
gtaggtgtcattctattctggggggtggggtggggcaggacagcaagggggaggattgggaagacaat
agcaggcatgctggggatgcggtgggctctatggcttctgaggcggaaagaaccagctggggctctag
ggggtatccccacgcgccctgtagcggcgcattaagcgcggcgggtgtggtggttacgcgcagcgtga
ccgctacacttgccagcgccctagcgcccgctcctttcgctttcttcccttcctttctcgccacgttc
gccggctttccccgtcaagctctaaatcgggggctccctttagggttccgatttagtgctttacggca
cctcgaccccaaaaaacttgattagggtgatggttcacgtagtgggccatcgccctgatagacggttt
ttcgccctttgacgttggagtccacgttctttaatagtggactcttgttccaaactggaacaacactc
aaccctatctcggtctattcttttgatttataagggattttgccgatttcggcctattggttaaaaaa
tgagctgatttaacaaaaatttaacgcgaattaattctgtggaatgtgtgtcagttagggtgtggaaa
gtccccaggctccccagcaggcagaagtatgcaaagcatgcatctcaattagtcagcaaccaggtgtg
gaaagtccccaggctccccagcaggcagaagtatgcaaagcatgcatctcaattagtcagcaaccata
gtcccgcccctaactccgcccatcccgcccctaactccgcccagttccgcccattctccgccccatgg
ctgactaattttttttatttatgcagaggccgaggccgcctctgcctctgagctattccagaagtagt
gaggaggcttttttggaggcctaggcttttgcaaaaagctcccgggagcttgtatatccattttcgga
tctgatcaagagacaggatgaggatcgtttcgcatgattgaacaagatggattgcacgcaggttctcc
ggccgcttgggtggagaggctattcggctatgactgggcacaacagacaatcggctgctctgatgccg
ccgtgttccggctgtcagcgcaggggcgcccggttctttttgtcaagaccgacctgtccggtgccctg
aatgaactgcaggacgaggcagcgcggctatcgtggctggccacgacgggcgttccttgcgcagctgt
gctcgacgttgtcactgaagcgggaagggactggctgctattgggcgaagtgccggggcaggatctcc
tgtcatctcaccttgctcctgccgagaaagtatccatcatggctgatgcaatgcggcggctgcatacg
cttgatccggctacctgcccattcgaccaccaagcgaaacatcgcatcgagcgagcacgtactcggat
ggaagccggtcttgtcgatcaggatgatctggacgaagagcatcaggggctcgcgccagccgaactgt
tcgccaggctcaaggcgcgcatgcccgacggcgaggatctcgtcgtgacccatggcgatgcctgcttg
ccgaatatcatggtggaaaatggccgcttttctggattcatcgactgtggccggctgggtgtggcgga
ccgctatcaggacatagcgttggctacccgtgatattgctgaagagcttggcggcgaatgggctgacc
gcttcctcgtgctttacggtatcgccgctcccgattcgcagcgcatcgccttctatcgccttcttgac
gagttcttctgagcgggactctggggttcgaaatgaccgaccaagcgacgcccaacctgccatcacga
gatttcgattccaccgccgccttctatgaaaggttgggcttcggaatcgttttccgggacgccggctg
gatgatcctccagcgcggggatctcatgctggagttcttcgcccaccccaacttgtttattgcagctt
ataatggttacaaataaagcaatagcatcacaaatttcacaaataaagcatttttttcactgcattct
agttgtggtttgtccaaactcatcaatgtatcttatcatgtctgtataccgtcgacctctagctagag
cttggcgtaatcatggtcatagctgtttcctgtgtgaaattgttatccgctcacaattccacacaaca
tacgagccggaagcataaagtgtaaagcctggggtgcctaatgagtgagctaactcacattaattgcg
ttgcgctcactgcccgctttccagtcgggaaacctgtcgtgccagctgcattaatgaatcggccaacg
cgcggggagaggcggtttgcgtattgggcgctcttccgcttcctcgctcactgactcgctgcgctcgg
tcgttcggctgcggcgagcggtatcagctcactcaaaggcggtaatacggttatccacagaatcaggg
gataacgcaggaaagaacatgtgagcaaaaggccagcaaaaggccaggaaccgtaaaaaggccgcgtt
gctggcgtttttccataggctccgcccccctgacgagcatcacaaaaatcgacgctcaagtcagaggt
ggcgaaacccgacaggactataaagataccaggcgtttccccctggaagctccctcgtgcgctctcct
gttccgaccctgccgcttaccggatacctgtccgcctttctcccttcgggaagcgtggcgctttctca
tagctcacgctgtaggtatctcagttcggtgtaggtcgttcgctccaagctgggctgtgtgcacgaac
cccccgttcagcccgaccgctgcgccttatccggtaactatcgtcttgagtccaacccggtaagacac
gacttatcgccactggcagcagccactggtaacaggattagcagagcgaggtatgtaggcggtgctac
agagttcttgaagtggtggcctaactacggctacactagaagaacagtatttggtatctgcgctctgc
tgaagccagttaccttcggaaaaagagttggtagctcttgatccggcaaacaaaccaccgctggtagc
ggtggtttttttgtttgcaagcagcagattacgcgcagaaaaaaaggatctcaagaagatcctttgat
cttttctacggggtctgacgctcagtggaacgaaaactcacgttaagggattttggtcatgagattat
caaaaaggatcttcacctagatccttttaaattaaaaatgaagttttaaatcaatctaaagtatatat
gagtaaacttggtctgacagttaccaatgcttaatcagtgaggcacctatctcagcgatctgtctatt
tcgttcatccatagttgcctgactccccgtcgtgtagataactacgatacgggagggcttaccatctg
gccccagtgctgcaatgataccgcgagacccacgctcaccggctccagatttatcagcaataaaccag
ccagccggaagggccgagcgcagaagtggtcctgcaactttatccgcctccatccagtctattaattg
ttgccgggaagctagagtaagtagttcgccagttaatagtttgcgcaacgttgttgccattgctacag
gcatcgtggtgtcacgctcgtcgtttggtatggcttcattcagctccggttcccaacgatcaaggcga
gttacatgatcccccatgttgtgcaaaaaagcggttagctccttcggtcctccgatcgttgtcagaag
taagttggccgcagtgttatcactcatggttatggcagcactgcataattctcttactgtcatgccat
ccgtaagatgcttttctgtgactggtgagtactcaaccaagtcattctgagaatagtgtatgcggcga
ccgagttgctcttgcccggcgtcaatacgggataataccgcgccacatagcagaactttaaaagtgct
catcattggaaaacgttcttcggggcgaaaactctcaaggatcttaccgctgttgagatccagttcga
tgtaacccactcgtgcacccaactgatcttcagcatcttttactttcaccagcgtttctgggtgagca
aaaacaggaaggcaaaatgccgcaaaaaagggaataagggcgacacggaaatgttgaatactcatact
cttcctttttcaatattattgaagcatttatcagggttattgtctcatgagcggatacatatttgaat
gtatttagaaaaataaacaaataggggttccgcgcacatttccccgaaaagtgccacctgacgtc
pVP29b-Indian_B.1.617_hexapro_UTR
SEQ ID NO: 80
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATgTTTGTGTTCCTGGTGCTGCTGCCACTGGTGTCCAGCCAGTGTGTGAACCTGACCACCAG
GACCCAACTTCCTCCTGCCTACACCAACTCCTTCACCAGGGGAGTCTACTACCCTGACAAGGTGTTCA
GGTCCTCTGTGCTGCACAGCACCCAGGACCTGTTCCTGCCATTCTTCAGCAATGTGACCTGGTTCCAT
GCCATCCATGTGTCTGGCACCAATGGCACCAAGAGGTTTGACAACCCTGTGCTGCCATTCAATGATGG
AGTCTACTTTGCCAGCACAGAGAAGAGCAACATCATCAGGGGCTGGATTTTTGGCACCACCCTGGACA
GCAAGACCCAGTCCCTGCTGATTGTGAACAATGCCACCAATGTGGTGATTAAGGTGTGTGAGTTCCAG
TTCTGTAATGACCCATTCCTGGACGTCTACTACCACAAGAACAACAAGTCCTGGATGAAGTCTGAGTT
CAGGGTCTACTCCTCTGCCAACAACTGTACCTTTGAATATGTGAGCCAACCATTCCTGATGGACTTGG
AGGGCAAGCAGGGCAACTTCAAGAACCTGAGGGAGTTTGTGTTCAAGAACATTGATGGCTACTTCAAG
ATTTACAGCAAACACACACCAATCAACCTGGTGAGGGACCTGCCACAGGGCTTCTCTGCCTTGGAACC
ACTGGTGGACCTGCCAATTGGCATCAACATCACCAGGTTCCAGACCCTGCTGGCTCTGCACAGGTCCT
ACCTGACACCTGGAGACTCCTCCTCTGGCTGGACAGCAGGAGCAGCAGCCTACTATGTGGGCTACCTC
CAACCAAGGACCTTCCTGCTGAAATACAATGAGAATGGCACCATCACAGATGCTGTGGACTGTGCCCT
GGACCCACTGTCTGAGACCAAGTGTACCCTGAAATCCTTCACAGTGGAGAAGGGCATCTACCAGACCA
GCAACTTCAGGGTCCAACCAACAGAGAGCATTGTGAGGTTTCCAAACATCACCAACCTGTGTCCATTT
GGAGAGGTGTTCAATGCCACCAGGTTTGCCTCTGTCTATGCCTGGAACAGGAAGAGGATTAGCAACTG
TGTGGCTGACTACTCTGTGCTCTACAACTCTGCCTCCTTCAGCACCTTCAAGTGTTATGGAGTGAGCC
CAACCAAACTGAATGACCTGTGTTTCACCAATGTCTATGCTGACTCCTTTGTGATTAGGGGAGATGAG
GTGAGACAGATTGCCCCTGGACAAACAGGCAAGATTGCTGACTACAACTACAAACTGCCTGATGACTT
CACAGGCTGTGTGATTGCCTGGAACAGCAACAACCTGGACAGCAAGGTGGGAGGCAACTACAACTACA
GGTACAGACTGTTCAGGAAGAGCAACCTGAAACCATTTGAGAGGGACATCAGCACAGAGATTTACCAG
GCTGGCAGCACACCATGTAATGGAGTGCAGGGCTTCAACTGTTACTTTCCACTCCAATCCTATGGCTT
CCAACCAACCAATGGAGTGGGCTACCAACCATACAGGGTGGTGGTGCTGTCCTTTGAACTGCTCCATG
CCCCTGCCACAGTGTGTGGACCAAAGAAGAGCACCAACCTGGTGAAGAACAAGTGTGTGAACTTCAAC
TTCAATGGACTGACAGGCACAGGAGTGCTGACAGAGAGCAACAAGAAGTTCCTGCCATTCCAACAGTT
TGGCAGGGACATTGCTGACACCACAGATGCTGTGAGGGACCCACAGACCTTGGAGATTCTGGACATCA
CACCATGTTCCTTTGGAGGAGTGTCTGTGATTACACCTGGCACCAACACCAGCAACCAGGTGGCTGTG
CTCTACCAGGGAGTGAACTGTACTGAGGTGCCTGTGGCTATCCATGCTGACCAACTTACACCAACCTG
GAGGGTCTACAGCACAGGCAGCAATGTGTTCCAGACCAGGGCTGGCTGTCTGATTGGAGCAGAGCATG
TGAACAACTCCTATGAGTGTGACATCCCAATTGGAGCAGGCATCTGTGCCTCCTACCAGACCCAGACC
AACAGCAGGGGCTCTGCATCTTCTGTGGCAAGCCAGAGCATCATTGCCTACACAATGAGTCTGGGAGC
AGAGAACTCTGTGGCTTACAGCAACAACAGCATTGCCATCCCAACCAACTTCACCATCTCTGTGACCA
CAGAGATTCTGCCTGTGAGTATGACCAAGACCTCTGTGGACTGTACAATGTATATCTGTGGAGACAGC
ACAGAGTGTAGCAACCTGCTGCTCCAATATGGCTCCTTCTGTACCCAACTTAACAGGGCTCTGACAGG
CATTGCTGTGGAACAGGACAAGAACACACAGGAGGTGTTTGCCCAGGTGAAGCAGATTTACAAGACAC
CTCCAATCAAGGACTTTGGAGGCTTCAACTTCAGCCAGATTCTGCCTGACCCAAGCAAGCCAAGCAAG
AGGTCCCCTATTGAGGACCTGCTGTTCAACAAGGTGACCCTGGCTGATGCTGGCTTCATCAAGCAATA
TGGAGACTGTCTGGGAGACATTGCTGCCAGGGACCTGATTTGTGCCCAGAAGTTCAATGGACTGACAG
TGCTGCCTCCACTGCTGACAGATGAGATGATTGCCCAATACACCTCTGCCCTGCTGGCTGGCACCATC
ACCTCTGGCTGGACCTTTGGAGCAGGACCAGCCCTCCAAATCCCATTTCCAATGCAGATGGCTTACAG
GTTCAATGGCATTGGAGTGACCCAGAATGTGCTCTATGAGAACCAGAAACTGATTGCCAACCAGTTCA
ACTCTGCCATTGGCAAGATTCAGGACTCCCTGTCCAGCACACCATCTGCCCTGGGCAAACTCCAAGAT
GTGGTGAACCAGAATGCCCAGGCTCTGAACACCCTGGTGAAGCAACTTTCCAGCAACTTTGGAGCCAT
CTCCTCTGTGCTGAATGACATCCTGAGCAGACTGGACCCACCAGAGGCTGAGGTCCAGATTGACAGAC
TGATTACAGGCAGACTCCAATCCCTCCAAACCTATGTGACCCAACAACTTATCAGGGCTGCTGAGATT
AGGGCATCTGCCAACCTGGCTGCCACCAAGATGAGTGAGTGTGTGCTGGGACAAAGCAAGAGGGTGGA
CTTCTGTGGCAAGGGCTACCACCTGATGAGTTTTCCACAGTCTGCCCCTCATGGAGTGGTGTTCCTGC
ATGTGACCTATGTGCCTGCCCATGAGAAGAACTTCACCACAGCCCCTGCCATCTGCCATGATGGCAAG
GCTCACTTTCCAAGGGAGGGAGTGTTTGTGAGCAATGGCACCCACTGGTTTGTGACCCAGAGGAACTT
CTATGAACCACAGATTATCACCACAGACAACACCTTTGTGTCTGGCAACTGTGATGTGGTGATTGGCA
TTGTGAACAACACAGTCTATGACCCACTCCAACCTGAACTGGACTCCTTCAAGGAGGAACTGGACAAA
TACTTCAAGAACCACACCAGCCCTGATGTGGACCTGGGAGACATCTCTGGCATCAATGCCTCTGTGGT
GAACATCCAGAAGGAGATTGACAGACTGAATGAGGTGGCTAAGAACCTGAATGAGTCCCTGATTGACC
TCCAAGAACTGGGCAAATATGAACAATACATCAAGTGGCCATGGTACATCTGGCTGGGCTTCATTGCT
GGACTGATTGCCATTGTGATGGTGACCATAATGCTGTGTTGTATGACCTCCTGTTGTTCCTGTCTGAA
AGGCTGTTGTTCCTGTGGCTCCTGTTGTAAGTTTGATGAGGATGACTCTGAACCTGTGCTGAAAGGAG
TGAAACTGCACTACACCTGACTCGAGCTGGTACTGCATGCACGCAATGCTAGCTGCCCCTTTCCCGTC
CTGGGTACCCCGAGTCTCCCCCGACCTCGGGTCCCAGGTATGCTCCCACCTCCACCTGCCCCACTCAC
CACCTCTGCTAGTTCCAGACACCTCCCAAGCACGCAGCAATGCAGCTCAAAACGCTTAGCCTAGCCAC
ACCCCCACGGGAAACAGCAGTGATTAACCTTTAGCAATAAACGAAAGTTTAACTAAGCTATACTAACC
CCAGGGTTGGTCAATTTCGTGCCAGCCACACCCTGGAGCTAGCaaaccgctgatcagcctcgactgtg
ccttctagttgccagccatctgttgtttgcccctcccccgtgccttccttgaccctggaaggtgccac
tcccactgtcctttcctaataaaatgaggaaattgcatcgcattgtctgagtaggtgtcattctattc
tggggggtggggtggggcaggacagcaagggggaggattgggaagacaatagcaggcatgctggggat
gcggtgggctctatggcttctgaggcggaaagaaccagctggggctctagggggtatccccacgcgcc
ctgtagcggcgcattaagcgcggcgggtgtggtggttacgcgcagcgtgaccgctacacttgccagcg
ccctagcgcccgctcctttcgctttcttcccttcctttctcgccacgttcgccggctttccccgtcaa
gctctaaatcgggggctccctttagggttccgatttagtgctttacggcacctcgaccccaaaaaact
tgattagggtgatggttcacgtagtgggccatcgccctgatagacggtttttcgccctttgacgttgg
agtccacgttctttaatagtggactcttgttccaaactggaacaacactcaaccctatctcggtctat
tcttttgatttataagggattttgccgatttcggcctattggttaaaaaatgagctgatttaacaaaa
atttaacgcgaattaattctgtggaatgtgtgtcagttagggtgtggaaagtccccaggctccccagc
aggcagaagtatgcaaagcatgcatctcaattagtcagcaaccaggtgtggaaagtccccaggctccc
cagcaggcagaagtatgcaaagcatgcatctcaattagtcagcaaccatagtcccgcccctaactccg
cccatcccgcccctaactccgcccagttccgcccattctccgccccatggctgactaattttttttat
ttatgcagaggccgaggccgcctctgcctctgagctattccagaagtagtgaggaggcttttttggag
gcctaggcttttgcaaaaagctcccgggagcttgtatatccattttcggatctgatcaagagacagga
tgaggatcgtttcgcatgattgaacaagatggattgcacgcaggttctccggccgcttgggtggagag
gctattcggctatgactgggcacaacagacaatcggctgctctgatgccgccgtgttccggctgtcag
cgcaggggcgcccggttctttttgtcaagaccgacctgtccggtgccctgaatgaactgcaggacgag
gcagcgcggctatcgtggctggccacgacgggcgttccttgcgcagctgtgctcgacgttgtcactga
agcgggaagggactggctgctattgggcgaagtgccggggcaggatctcctgtcatctcaccttgctc
ctgccgagaaagtatccatcatggctgatgcaatgcggcggctgcatacgcttgatccggctacctgc
ccattcgaccaccaagcgaaacatcgcatcgagcgagcacgtactcggatggaagccggtcttgtcga
tcaggatgatctggacgaagagcatcaggggctcgcgccagccgaactgttcgccaggctcaaggcgc
gcatgcccgacggcgaggatctcgtcgtgacccatggcgatgcctgcttgccgaatatcatggtggaa
aatggccgcttttctggattcatcgactgtggccggctgggtgtggcggaccgctatcaggacatagc
gttggctacccgtgatattgctgaagagcttggcggcgaatgggctgaccgcttcctcgtgctttacg
gtatcgccgctcccgattcgcagcgcatcgccttctatcgccttcttgacgagttcttctgagcggga
ctctggggttcgaaatgaccgaccaagcgacgcccaacctgccatcacgagatttcgattccaccgcc
gccttctatgaaaggttgggcttcggaatcgttttccgggacgccggctggatgatcctccagcgcgg
ggatctcatgctggagttcttcgcccaccccaacttgtttattgcagcttataatggttacaaataaa
gcaatagcatcacaaatttcacaaataaagcatttttttcactgcattctagttgtggtttgtccaaa
ctcatcaatgtatcttatcatgtctgtataccgtcgacctctagctagagcttggcgtaatcatggtc
atagctgtttcctgtgtgaaattgttatccgctcacaattccacacaacatacgagccggaagcataa
agtgtaaagcctggggtgcctaatgagtgagctaactcacattaattgcgttgcgctcactgcccgct
ttccagtcgggaaacctgtcgtgccagctgcattaatgaatcggccaacgcgcggggagaggcggttt
gcgtattgggcgctcttccgcttcctcgctcactgactcgctgcgctcggtcgttcggctgcggcgag
cggtatcagctcactcaaaggcggtaatacggttatccacagaatcaggggataacgcaggaaagaac
atgtgagcaaaaggccagcaaaaggccaggaaccgtaaaaaggccgcgttgctggcgtttttccatag
gctccgcccccctgacgagcatcacaaaaatcgacgctcaagtcagaggtggcgaaacccgacaggac
tataaagataccaggcgtttccccctggaagctccctcgtgcgctctcctgttccgaccctgccgctt
accggatacctgtccgcctttctcccttcgggaagcgtggcgctttctcatagctcacgctgtaggta
tctcagttcggtgtaggtcgttcgctccaagctgggctgtgtgcacgaaccccccgttcagcccgacc
gctgcgccttatccggtaactatcgtcttgagtccaacccggtaagacacgacttatcgccactggca
gcagccactggtaacaggattagcagagcgaggtatgtaggcggtgctacagagttcttgaagtggtg
gcctaactacggctacactagaagaacagtatttggtatctgcgctctgctgaagccagttaccttcg
gaaaaagagttggtagctcttgatccggcaaacaaaccaccgctggtagcggtggtttttttgtttgc
aagcagcagattacgcgcagaaaaaaaggatctcaagaagatcctttgatcttttctacggggtctga
cgctcagtggaacgaaaactcacgttaagggattttggtcatgagattatcaaaaaggatcttcacct
agatccttttaaattaaaaatgaagttttaaatcaatctaaagtatatatgagtaaacttggtctgac
agttaccaatgcttaatcagtgaggcacctatctcagcgatctgtctatttcgttcatccatagttgc
ctgactccccgtcgtgtagataactacgatacgggagggcttaccatctggccccagtgctgcaatga
taccgcgagacccacgctcaccggctccagatttatcagcaataaaccagccagccggaagggccgag
cgcagaagtggtcctgcaactttatccgcctccatccagtctattaattgttgccgggaagctagagt
aagtagttcgccagttaatagtttgcgcaacgttgttgccattgctacaggcatcgtggtgtcacgct
cgtcgtttggtatggcttcattcagctccggttcccaacgatcaaggcgagttacatgatcccccatg
ttgtgcaaaaaagcggttagctccttcggtcctccgatcgttgtcagaagtaagttggccgcagtgtt
atcactcatggttatggcagcactgcataattctcttactgtcatgccatccgtaagatgcttttctg
tgactggtgagtactcaaccaagtcattctgagaatagtgtatgcggcgaccgagttgctcttgcccg
gcgtcaatacgggataataccgcgccacatagcagaactttaaaagtgctcatcattggaaaacgttc
ttcggggcgaaaactctcaaggatcttaccgctgttgagatccagttcgatgtaacccactcgtgcac
ccaactgatcttcagcatcttttactttcaccagcgtttctgggtgagcaaaaacaggaaggcaaaat
gccgcaaaaaagggaataagggcgacacggaaatgttgaatactcatactcttcctttttcaatatta
ttgaagcatttatcagggttattgtctcatgagcggatacatatttgaatgtatttagaaaaataaac
aaataggggttccgcgcacatttccccgaaaagtgccacctgacgtc
pVP33-SARS-CoV-UTR
SEQ ID NO: 81
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATgTTCATCTTCCTGCTGTTCCTGACCCTGACCTCTGGCTCTGACCTGGACAGGTGTACCAC
CTTTGATGATGTCCAGGCTCCAAACTACACCCAACACACCTCCAGTATGAGGGGAGTCTACTACCCTG
ATGAGATTTTCAGGTCTGACACCCTCTACCTGACCCAGGACCTGTTCCTGCCATTCTACAGCAATGTG
ACAGGCTTCCACACCATCAACCACACCTTTGACAACCCTGTGATTCCATTCAAGGATGGCATCTACTT
TGCTGCCACAGAGAAGAGCAATGTGGTGAGGGGCTGGGTGTTTGGCAGCACAATGAACAACAAGAGCC
AGTCTGTGATTATCATCAACAACAGCACCAATGTGGTGATTAGGGCTTGTAACTTTGAACTGTGTGAC
AACCCATTCTTTGCTGTGAGCAAGCCTATGGGCACCCAGACCCACACAATGATTTTTGACAATGCCTT
CAACTGTACCTTTGAATACATCTCTGATGCCTTCTCCCTGGATGTGTCTGAGAAGTCTGGCAACTTCA
AACACCTGAGGGAGTTTGTGTTCAAGAACAAGGATGGCTTCCTCTATGTCTACAAGGGCTACCAACCA
ATTGATGTGGTGAGGGACCTGCCATCTGGCTTCAACACCCTGAAACCAATCTTCAAACTGCCACTGGG
CATCAACATCACCAACTTCAGGGCTATCCTGACAGCCTTCAGCCCTGCCCAGGACACCTGGGGCACCT
CTGCTGCTGCCTACTTTGTGGGCTACCTGAAACCAACCACCTTTATGCTGAAATATGATGAGAATGGC
ACCATCACAGATGCTGTGGACTGTAGCCAGAACCCACTGGCTGAACTGAAATGTTCTGTGAAGTCCTT
TGAGATTGACAAGGGCATCTACCAGACCAGCAACTTCAGGGTGGTGCCATCTGGAGATGTGGTGAGGT
TTCCAAACATCACCAACCTGTGTCCATTTGGAGAGGTGTTCAATGCCACCAAGTTTCCATCTGTCTAT
GCCTGGGAGAGGAAGAAGATTAGCAACTGTGTGGCTGACTACTCTGTGCTCTACAACAGCACCTTCTT
CAGCACCTTCAAGTGTTATGGAGTGTCTGCCACCAAACTGAATGACCTGTGTTTCAGCAATGTCTATG
CTGACTCCTTTGTGGTGAAGGGAGATGATGTGAGACAGATTGCCCCTGGACAAACAGGAGTGATTGCT
GACTACAACTACAAACTGCCTGATGACTTTATGGGCTGTGTGCTGGCTTGGAACACCAGGAACATTGA
TGCCACCAGCACAGGCAACTACAACTACAAATACAGATACCTGAGACATGGCAAACTGAGACCATTTG
AGAGGGACATCAGCAATGTGCCATTCAGCCCTGATGGCAAGCCATGTACTCCTCCTGCCCTGAACTGT
TACTGGCCACTGAATGACTATGGCTTCTACACCACCACAGGCATTGGCTACCAACCATACAGGGTGGT
GGTGCTGTCCTTTGAACTGCTGAATGCCCCTGCCACAGTGTGTGGACCAAAACTGAGCACAGACCTGA
TTAAGAACCAGTGTGTGAACTTCAACTTCAATGGACTGACAGGCACAGGAGTGCTGACACCATCCAGC
AAGAGGTTCCAACCATTCCAACAGTTTGGCAGGGATGTGTCTGACTTCACAGACTCTGTGAGGGACCC
AAAGACCTCTGAGATTCTGGACATCAGCCCATGTTCCTTTGGAGGAGTGTCTGTGATTACACCTGGCA
CCAATGCCTCCTCTGAGGTGGCTGTGCTCTACCAGGATGTGAACTGTACTGATGTGAGCACAGCCATC
CATGCTGACCAACTTACACCTGCCTGGAGGATTTACAGCACAGGCAACAATGTGTTCCAGACCCAGGC
TGGCTGTCTGATTGGAGCAGAGCATGTGGACACCTCCTATGAGTGTGACATCCCAATTGGAGCAGGCA
TCTGTGCCTCCTACCACACAGTGTCCCTGCTGAGGAGCACCAGCCAGAAGAGCATTGTGGCTTACACA
ATGAGTCTGGGAGCAGACTCCAGCATTGCCTACAGCAACAACACCATTGCCATCCCAACCAACTTCAG
CATCAGCATCACCACAGAGGTGATGCCTGTGAGTATGGCTAAGACCTCTGTGGACTGTAATATGTATA
TCTGTGGAGACAGCACAGAGTGTGCCAACCTGCTGCTCCAATATGGCTCCTTCTGTACCCAACTTAAC
AGGGCTCTGTCTGGCATTGCTGCTGAACAGGACAGGAACACCAGGGAGGTGTTTGCCCAGGTGAAGCA
GATGTATAAGACACCAACCCTGAAATACTTTGGAGGCTTCAACTTCAGCCAGATTCTGCCTGACCCAC
TGAAACCAACCAAGAGGTCCTTCATTGAGGACCTGCTGTTCAACAAGGTGACCCTGGCTGATGCTGGC
TTTATGAAGCAATATGGAGAGTGTCTGGGAGACATCAATGCCAGGGACCTGATTTGTGCCCAGAAGTT
CAATGGACTGACAGTGCTGCCTCCACTGCTGACAGATGATATGATTGCTGCCTACACAGCAGCCCTGG
TGTCTGGCACAGCCACAGCAGGCTGGACCTTTGGAGCAGGAGCAGCCCTCCAAATCCCATTTGCTATG
CAGATGGCTTACAGGTTCAATGGCATTGGAGTGACCCAGAATGTGCTCTATGAGAACCAGAAGCAGAT
TGCCAACCAGTTCAACAAGGCTATCAGCCAGATTCAGGAGTCCCTGACCACCACCAGCACAGCCCTGG
GCAAACTCCAAGATGTGGTGAACCAGAATGCCCAGGCTCTGAACACCCTGGTGAAGCAACTTTCCAGC
AACTTTGGAGCCATCTCCTCTGTGCTGAATGACATCCTGAGCAGACTGGACAAGGTGGAGGCTGAGGT
CCAGATTGACAGACTGATTACAGGCAGACTCCAATCCCTCCAAACCTATGTGACCCAACAACTTATCA
GGGCTGCTGAGATTAGGGCATCTGCCAACCTGGCTGCCACCAAGATGAGTGAGTGTGTGCTGGGACAA
AGCAAGAGGGTGGACTTCTGTGGCAAGGGCTACCACCTGATGAGTTTTCCACAGGCTGCCCCTCATGG
AGTGGTGTTCCTGCATGTGACCTATGTGCCAAGCCAGGAGAGGAACTTCACCACAGCCCCTGCCATCT
GCCATGAGGGCAAGGCTTACTTTCCAAGGGAGGGAGTGTTTGTGTTCAATGGCACCTCCTGGTTCATC
ACCCAGAGGAACTTCTTCAGCCCACAGATTATCACCACAGACAACACCTTTGTGTCTGGCAACTGTGA
TGTGGTGATTGGCATCATCAACAACACAGTCTATGACCCACTCCAACCTGAACTGGACTCCTTCAAGG
AGGAACTGGACAAATACTTCAAGAACCACACCAGCCCTGATGTGGACCTGGGAGACATCTCTGGCATC
AATGCCTCTGTGGTGAACATCCAGAAGGAGATTGACAGACTGAATGAGGTGGCTAAGAACCTGAATGA
GTCCCTGATTGACCTCCAAGAACTGGGCAAATATGAACAATACATCAAGTGGCCATGGTATGTGTGGC
TGGGCTTCATTGCTGGACTGATTGCCATTGTGATGGTGACCATCCTGCTGTGTTGTATGACCTCCTGT
TGTTCCTGTCTGAAAGGAGCCTGTTCCTGTGGCTCCTGTTGTAAGTTTGATGAGGATGACTCTGAACC
TGTGCTGAAAGGAGTGAAACTGCACTACACCTAACTCGAGCTGGTACTGCATGCACGCAATGCTAGCT
GCCCCTTTCCCGTCCTGGGTACCCCGAGTCTCCCCCGACCTCGGGTCCCAGGTATGCTCCCACCTCCA
CCTGCCCCACTCACCACCTCTGCTAGTTCCAGACACCTCCCAAGCACGCAGCAATGCAGCTCAAAACG
CTTAGCCTAGCCACACCCCCACGGGAAACAGCAGTGATTAACCTTTAGCAATAAACGAAAGTTTAACT
AAGCTATACTAACCCCAGGGTTGGTCAATTTCGTGCCAGCCACACCCTGGAGCTAGCaaaccgctgat
cagcctcgactgtgccttctagttgccagccatctgttgtttgcccctcccccgtgccttccttgacc
ctggaaggtgccactcccactgtcctttcctaataaaatgaggaaattgcatcgcattgtctgagtag
gtgtcattctattctggggggtggggtggggcaggacagcaagggggaggattgggaagacaatagca
ggcatgctggggatgcggtgggctctatggcttctgaggcggaaagaaccagctggggctctaggggg
tatccccacgcgccctgtagcggcgcattaagcgcggcgggtgtggtggttacgcgcagcgtgaccgc
tacacttgccagcgccctagcgcccgctcctttcgctttcttcccttcctttctcgccacgttcgccg
gctttccccgtcaagctctaaatcgggggctccctttagggttccgatttagtgctttacggcacctc
gaccccaaaaaacttgattagggtgatggttcacgtagtgggccatcgccctgatagacggtttttcg
ccctttgacgttggagtccacgttctttaatagtggactcttgttccaaactggaacaacactcaacc
ctatctcggtctattcttttgatttataagggattttgccgatttcggcctattggttaaaaaatgag
ctgatttaacaaaaatttaacgcgaattaattctgtggaatgtgtgtcagttagggtgtggaaagtcc
ccaggctccccagcaggcagaagtatgcaaagcatgcatctcaattagtcagcaaccaggtgtggaaa
gtccccaggctccccagcaggcagaagtatgcaaagcatgcatctcaattagtcagcaaccatagtcc
cgcccctaactccgcccatcccgcccctaactccgcccagttccgcccattctccgccccatggctga
ctaattttttttatttatgcagaggccgaggccgcctctgcctctgagctattccagaagtagtgagg
aggcttttttggaggcctaggcttttgcaaaaagctcccgggagcttgtatatccattttcggatctg
atcaagagacaggatgaggatcgtttcgcatgattgaacaagatggattgcacgcaggttctccggcc
gcttgggtggagaggctattcggctatgactgggcacaacagacaatcggctgctctgatgccgccgt
gttccggctgtcagcgcaggggcgcccggttctttttgtcaagaccgacctgtccggtgccctgaatg
aactgcaggacgaggcagcgcggctatcgtggctggccacgacgggcgttccttgcgcagctgtgctc
gacgttgtcactgaagcgggaagggactggctgctattgggcgaagtgccggggcaggatctcctgtc
atctcaccttgctcctgccgagaaagtatccatcatggctgatgcaatgcggcggctgcatacgcttg
atccggctacctgcccattcgaccaccaagcgaaacatcgcatcgagcgagcacgtactcggatggaa
gccggtcttgtcgatcaggatgatctggacgaagagcatcaggggctcgcgccagccgaactgttcgc
caggctcaaggcgcgcatgcccgacggcgaggatctcgtcgtgacccatggcgatgcctgcttgccga
atatcatggtggaaaatggccgcttttctggattcatcgactgtggccggctgggtgtggcggaccgc
tatcaggacatagcgttggctacccgtgatattgctgaagagcttggcggcgaatgggctgaccgctt
cctcgtgctttacggtatcgccgctcccgattcgcagcgcatcgccttctatcgccttcttgacgagt
tcttctgagcgggactctggggttcgaaatgaccgaccaagcgacgcccaacctgccatcacgagatt
tcgattccaccgccgccttctatgaaaggttgggcttcggaatcgttttccgggacgccggctggatg
atcctccagcgcggggatctcatgctggagttcttcgcccaccccaacttgtttattgcagcttataa
tggttacaaataaagcaatagcatcacaaatttcacaaataaagcatttttttcactgcattctagtt
gtggtttgtccaaactcatcaatgtatcttatcatgtctgtataccgtcgacctctagctagagcttg
gcgtaatcatggtcatagctgtttcctgtgtgaaattgttatccgctcacaattccacacaacatacg
agccggaagcataaagtgtaaagcctggggtgcctaatgagtgagctaactcacattaattgcgttgc
gctcactgcccgctttccagtcgggaaacctgtcgtgccagctgcattaatgaatcggccaacgcgcg
gggagaggcggtttgcgtattgggcgctcttccgcttcctcgctcactgactcgctgcgctcggtcgt
tcggctgcggcgagcggtatcagctcactcaaaggcggtaatacggttatccacagaatcaggggata
acgcaggaaagaacatgtgagcaaaaggccagcaaaaggccaggaaccgtaaaaaggccgcgttgctg
gcgtttttccataggctccgcccccctgacgagcatcacaaaaatcgacgctcaagtcagaggtggcg
aaacccgacaggactataaagataccaggcgtttccccctggaagctccctcgtgcgctctcctgttc
cgaccctgccgcttaccggatacctgtccgcctttctcccttcgggaagcgtggcgctttctcatagc
tcacgctgtaggtatctcagttcggtgtaggtcgttcgctccaagctgggctgtgtgcacgaaccccc
cgttcagcccgaccgctgcgccttatccggtaactatcgtcttgagtccaacccggtaagacacgact
tatcgccactggcagcagccactggtaacaggattagcagagcgaggtatgtaggcggtgctacagag
ttcttgaagtggtggcctaactacggctacactagaagaacagtatttggtatctgcgctctgctgaa
gccagttaccttcggaaaaagagttggtagctcttgatccggcaaacaaaccaccgctggtagcggtg
gtttttttgtttgcaagcagcagattacgcgcagaaaaaaaggatctcaagaagatcctttgatcttt
tctacggggtctgacgctcagtggaacgaaaactcacgttaagggattttggtcatgagattatcaaa
aaggatcttcacctagatccttttaaattaaaaatgaagttttaaatcaatctaaagtatatatgagt
aaacttggtctgacagttaccaatgcttaatcagtgaggcacctatctcagcgatctgtctatttcgt
tcatccatagttgcctgactccccgtcgtgtagataactacgatacgggagggcttaccatctggccc
cagtgctgcaatgataccgcgagacccacgctcaccggctccagatttatcagcaataaaccagccag
ccggaagggccgagcgcagaagtggtcctgcaactttatccgcctccatccagtctattaattgttgc
cgggaagctagagtaagtagttcgccagttaatagtttgcgcaacgttgttgccattgctacaggcat
cgtggtgtcacgctcgtcgtttggtatggcttcattcagctccggttcccaacgatcaaggcgagtta
catgatcccccatgttgtgcaaaaaagcggttagctccttcggtcctccgatcgttgtcagaagtaag
ttggccgcagtgttatcactcatggttatggcagcactgcataattctcttactgtcatgccatccgt
aagatgcttttctgtgactggtgagtactcaaccaagtcattctgagaatagtgtatgcggcgaccga
gttgctcttgcccggcgtcaatacgggataataccgcgccacatagcagaactttaaaagtgctcatc
attggaaaacgttcttcggggcgaaaactctcaaggatcttaccgctgttgagatccagttcgatgta
acccactcgtgcacccaactgatcttcagcatcttttactttcaccagcgtttctgggtgagcaaaaa
caggaaggcaaaatgccgcaaaaaagggaataagggcgacacggaaatgttgaatactcatactcttc
ctttttcaatattattgaagcatttatcagggttattgtctcatgagcggatacatatttgaatgtat
ttagaaaaataaacaaataggggttccgcgcacatttccccgaaaagtgccacctgacgtc
pVP39_SARS-CoV-2-UTR-spike_delta_HexaPro_Circular
SEQ ID NO: 82
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccGAGAATAAACTAGTATTCTTCTGGTCCCCACAGACTCAGAGAGAACCC
GCCACCATgTTTGTGTTCCTGGTGCTGCTGCCACTGGTGTCCAGCCAGTGTGTGAACCTGAGGACCAG
GACCCAACTTCCTCCTGCCTACACCAACTCCTTCACCAGGGGAGTCTACTACCCTGACAAGGTGTTCA
GGTCCTCTGTGCTGCACAGCACCCAGGACCTGTTCCTGCCATTCTTCAGCAATGTGACCTGGTTCCAT
GCCATCCATGTGTCTGGCACCAATGGCACCAAGAGGTTTGACAACCCTGTGCTGCCATTCAATGATGG
AGTCTACTTTGCCAGCACAGAGAAGAGCAACATCATCAGGGGCTGGATTTTTGGCACCACCCTGGACA
GCAAGACCCAGTCCCTGCTGATTGTGAACAATGCCACCAATGTGGTGATTAAGGTGTGTGAGTTCCAG
TTCTGTAATGACCCATTCCTGGACGTCTACTACCACAAGAACAACAAGTCCTGGATGGAGTCTGGCGT
CTACTCCTCTGCCAACAACTGTACCTTTGAATATGTGAGCCAACCATTCCTGATGGACTTGGAGGGCA
AGCAGGGCAACTTCAAGAACCTGAGGGAGTTTGTGTTCAAGAACATTGATGGCTACTTCAAGATTTAC
AGCAAACACACACCAATCAACCTGGTGAGGGACCTGCCACAGGGCTTCTCTGCCTTGGAACCACTGGT
GGACCTGCCAATTGGCATCAACATCACCAGGTTCCAGACCCTGCTGGCTCTGCACAGGTCCTACCTGA
CACCTGGAGACTCCTCCTCTGGCTGGACAGCAGGAGCAGCAGCCTACTATGTGGGCTACCTCCAACCA
AGGACCTTCCTGCTGAAATACAATGAGAATGGCACCATCACAGATGCTGTGGACTGTGCCCTGGACCC
ACTGTCTGAGACCAAGTGTACCCTGAAATCCTTCACAGTGGAGAAGGGCATCTACCAGACCAGCAACT
TCAGGGTCCAACCAACAGAGAGCATTGTGAGGTTTCCAAACATCACCAACCTGTGTCCATTTGGAGAG
GTGTTCAATGCCACCAGGTTTGCCTCTGTCTATGCCTGGAACAGGAAGAGGATTAGCAACTGTGTGGC
TGACTACTCTGTGCTCTACAACTCTGCCTCCTTCAGCACCTTCAAGTGTTATGGAGTGAGCCCAACCA
AACTGAATGACCTGTGTTTCACCAATGTCTATGCTGACTCCTTTGTGATTAGGGGAGATGAGGTGAGA
CAGATTGCCCCTGGACAAACAGGCAAGATTGCTGACTACAACTACAAACTGCCTGATGACTTCACAGG
CTGTGTGATTGCCTGGAACAGCAACAACCTGGACAGCAAGGTGGGAGGCAACTACAACTACAGGTACA
GACTGTTCAGGAAGAGCAACCTGAAACCATTTGAGAGGGACATCAGCACAGAGATTTACCAGGCTGGC
AGCAAGCCATGTAATGGAGTGGAGGGCTTCAACTGTTACTTTCCACTCCAATCCTATGGCTTCCAACC
AACCAATGGAGTGGGCTACCAACCATACAGGGTGGTGGTGCTGTCCTTTGAACTGCTCCATGCCCCTG
CCACAGTGTGTGGACCAAAGAAGAGCACCAACCTGGTGAAGAACAAGTGTGTGAACTTCAACTTCAAT
GGACTGACAGGCACAGGAGTGCTGACAGAGAGCAACAAGAAGTTCCTGCCATTCCAACAGTTTGGCAG
GGACATTGCTGACACCACAGATGCTGTGAGGGACCCACAGACCTTGGAGATTCTGGACATCACACCAT
GTTCCTTTGGAGGAGTGTCTGTGATTACACCTGGCACCAACACCAGCAACCAGGTGGCTGTGCTCTAC
CAGGGCGTGAACTGTACTGAGGTGCCTGTGGCTATCCATGCTGACCAACTTACACCAACCTGGAGGGT
CTACAGCACAGGAAGCAACGTGTTCCAGACCAGGGCTGGCTGTCTGATTGGAGCAGAGCATGTGAACA
ACTCCTATGAGTGTGACATCCCAATTGGAGCAGGCATCTGTGCCTCCTACCAGACCCAGACCAACAGC
AGGGGCTCTGCATCTTCTGTGGCAAGCCAGAGCATCATTGCCTACACAATGAGTCTGGGAGCAGAGAA
CTCTGTGGCTTACAGCAACAACAGCATTGCCATCCCAACCAACTTCACCATCTCTGTGACCACAGAGA
TTCTGCCTGTGAGTATGACCAAGACCTCTGTGGACTGTACAATGTATATCTGTGGAGACAGCACAGAG
TGTAGCAACCTGCTGCTCCAATATGGCTCCTTCTGTACCCAACTTAACAGGGCTCTGACAGGCATTGC
TGTGGAACAGGACAAGAACACCCAGGAGGTGTTTGCCCAGGTGAAGCAGATTTACAAGACACCTCCAA
TCAAGGACTTTGGAGGCTTCAACTTCAGCCAGATTCTGCCTGACCCAAGCAAGCCAAGCAAGAGGTCC
CCTATTGAGGACCTGCTGTTCAACAAGGTGACCCTGGCTGATGCTGGCTTCATCAAGCAATATGGAGA
CTGTCTGGGAGACATTGCCGCCAGGGACCTGATTTGTGCCCAGAAGTTCAATGGACTGACAGTGCTGC
CTCCACTGCTGACAGATGAGATGATTGCCCAATACACCTCTGCCCTGCTGGCTGGCACCATCACCTCT
GGCTGGACCTTTGGAGCAGGACCAGCCCTCCAAATCCCATTTCCAATGCAGATGGCTTACAGGTTCAA
TGGCATTGGAGTGACCCAGAATGTGCTCTATGAGAACCAGAAACTGATTGCCAACCAGTTCAACTCTG
CCATTGGCAAGATTCAGGACTCCCTGTCCAGCACACCATCTGCCCTGGGCAAACTCCAAAACGTGGTG
AACCAGAATGCCCAGGCTCTGAACACCCTGGTGAAGCAACTTTCCAGCAACTTTGGAGCCATCTCCTC
TGTGCTGAATGACATCCTGAGCAGACTGGACCCACCAGAGGCTGAGGTCCAGATTGACAGACTGATTA
CAGGCAGACTCCAATCCCTCCAAACCTATGTGACCCAACAACTTATCAGGGCTGCTGAGATTAGGGCA
TCTGCCAACCTGGCTGCCACCAAGATGAGTGAGTGTGTGCTGGGACAAAGCAAGAGGGTGGACTTCTG
TGGCAAGGGCTACCACCTGATGAGTTTTCCACAGTCTGCCCCTCATGGAGTGGTGTTCCTGCATGTGA
CCTATGTGCCTGCCCAGGAGAAGAACTTCACCACAGCCCCTGCCATCTGCCATGATGGCAAGGCTCAC
TTTCCAAGGGAGGGAGTGTTTGTGAGCAATGGCACCCACTGGTTTGTGACCCAGAGGAACTTCTATGA
ACCACAGATTATCACCACAGACAACACCTTTGTGTCTGGCAACTGTGATGTGGTGATTGGCATTGTGA
ACAACACAGTCTATGACCCACTCCAACCTGAACTGGACTCCTTCAAGGAGGAACTGGACAAATACTTC
AAGAACCACACCAGCCCTGATGTGGACCTGGGAGACATCTCTGGCATCAATGCCTCTGTGGTGAACAT
CCAGAAGGAGATTGACAGACTGAATGAGGTGGCTAAGAACCTGAATGAGTCCCTGATTGACCTCCAAG
AACTGGGCAAATATGAACAATACATCAAGTGGCCATGGTACATCTGGCTGGGCTTCATTGCTGGACTG
ATTGCCATTGTGATGGTGACCATAATGCTGTGTTGTATGACCTCCTGTTGTTCCTGTCTGAAAGGCTG
TTGTTCCTGTGGCTCCTGTTGTAAGTTTGATGAGGATGACTCTGAACCTGTGCTGAAAGGAGTGAAAC
TGCACTACACCTGACTCGAGCTGGTACTGCATGCACGCAATGCTAGCTGCCCCTTTCCCGTCCTGGGT
ACCCCGAGTCTCCCCCGACCTCGGGTCCCAGGTATGCTCCCACCTCCACCTGCCCCACTCACCACCTC
TGCTAGTTCCAGACACCTCCCAAGCACGCAGCAATGCAGCTCAAAACGCTTAGCCTAGCCACACCCCC
ACGGGAAACAGCAGTGATTAACCTTTAGCAATAAACGAAAGTTTAACTAAGCTATACTAACCCCAGGG
TTGGTCAATTTCGTGCCAGCCACACCCTGGAGCTAGCaaaccgctgatcagcctcgactgtgccttct
agttgccagccatctgttgtttgcccctcccccgtgccttccttgaccctggaaggtgccactcccac
tgtcctttcctaataaaatgaggaaattgcatcgcattgtctgagtaggtgtcattctattctggggg
gtggggtggggcaggacagcaagggggaggattgggaagacaatagcaggcatgctggggatgcggtg
ggctctatggcttctgaggcggaaagaaccagctggggctctagggggtatccccacgcgccctgtag
cggcgcattaagcgcggcgggtgtggtggttacgcgcagcgtgaccgctacacttgccagcgccctag
cgcccgctcctttcgctttcttcccttcctttctcgccacgttcgccggctttccccgtcaagctcta
aatcgggggctccctttagggttccgatttagtgctttacggcacctcgaccccaaaaaacttgatta
gggtgatggttcacgtagtgggccatcgccctgatagacggtttttcgccctttgacgttggagtcca
cgttctttaatagtggactcttgttccaaactggaacaacactcaaccctatctcggtctattctttt
gatttataagggattttgccgatttcggcctattggttaaaaaatgagctgatttaacaaaaatttaa
cgcgaattaattctgtggaatgtgtgtcagttagggtgtggaaagtccccaggctccccagcaggcag
aagtatgcaaagcatgcatctcaattagtcagcaaccaggtgtggaaagtccccaggctccccagcag
gcagaagtatgcaaagcatgcatctcaattagtcagcaaccatagtcccgcccctaactccgcccatc
ccgcccctaactccgcccagttccgcccattctccgccccatggctgactaattttttttatttatgc
agaggccgaggccgcctctgcctctgagctattccagaagtagtgaggaggcttttttggaggcctag
gcttttgcaaaaagctcccgggagcttgtatatccattttcggatctgatcaagagacaggatgagga
tcgtttcgcatgattgaacaagatggattgcacgcaggttctccggccgcttgggtggagaggctatt
cggctatgactgggcacaacagacaatcggctgctctgatgccgccgtgttccggctgtcagcgcagg
ggcgcccggttctttttgtcaagaccgacctgtccggtgccctgaatgaactgcaggacgaggcagcg
cggctatcgtggctggccacgacgggcgttccttgcgcagctgtgctcgacgttgtcactgaagcggg
aagggactggctgctattgggcgaagtgccggggcaggatctcctgtcatctcaccttgctcctgccg
agaaagtatccatcatggctgatgcaatgcggcggctgcatacgcttgatccggctacctgcccattc
gaccaccaagcgaaacatcgcatcgagcgagcacgtactcggatggaagccggtcttgtcgatcagga
tgatctggacgaagagcatcaggggctcgcgccagccgaactgttcgccaggctcaaggcgcgcatgc
ccgacggcgaggatctcgtcgtgacccatggcgatgcctgcttgccgaatatcatggtggaaaatggc
cgcttttctggattcatcgactgtggccggctgggtgtggcggaccgctatcaggacatagcgttggc
tacccgtgatattgctgaagagcttggcggcgaatgggctgaccgcttcctcgtgctttacggtatcg
ccgctcccgattcgcagcgcatcgccttctatcgccttcttgacgagttcttctgagcgggactctgg
ggttcgaaatgaccgaccaagcgacgcccaacctgccatcacgagatttcgattccaccgccgccttc
tatgaaaggttgggcttcggaatcgttttccgggacgccggctggatgatcctccagcgcggggatct
catgctggagttcttcgcccaccccaacttgtttattgcagcttataatggttacaaataaagcaata
gcatcacaaatttcacaaataaagcatttttttcactgcattctagttgtggtttgtccaaactcatc
aatgtatcttatcatgtctgtataccgtcgacctctagctagagcttggcgtaatcatggtcatagct
gtttcctgtgtgaaattgttatccgctcacaattccacacaacatacgagccggaagcataaagtgta
aagcctggggtgcctaatgagtgagctaactcacattaattgcgttgcgctcactgcccgctttccag
tcgggaaacctgtcgtgccagctgcattaatgaatcggccaacgcgcggggagaggcggtttgcgtat
tgggcgctcttccgcttcctcgctcactgactcgctgcgctcggtcgttcggctgcggcgagcggtat
cagctcactcaaaggcggtaatacggttatccacagaatcaggggataacgcaggaaagaacatgtga
gcaaaaggccagcaaaaggccaggaaccgtaaaaaggccgcgttgctggcgtttttccataggctccg
cccccctgacgagcatcacaaaaatcgacgctcaagtcagaggtggcgaaacccgacaggactataaa
gataccaggcgtttccccctggaagctccctcgtgcgctctcctgttccgaccctgccgcttaccgga
tacctgtccgcctttctcccttcgggaagcgtggcgctttctcatagctcacgctgtaggtatctcag
ttcggtgtaggtcgttcgctccaagctgggctgtgtgcacgaaccccccgttcagcccgaccgctgcg
ccttatccggtaactatcgtcttgagtccaacccggtaagacacgacttatcgccactggcagcagcc
actggtaacaggattagcagagcgaggtatgtaggcggtgctacagagttcttgaagtggtggcctaa
ctacggctacactagaagaacagtatttggtatctgcgctctgctgaagccagttaccttcggaaaaa
gagttggtagctcttgatccggcaaacaaaccaccgctggtagcggtggtttttttgtttgcaagcag
cagattacgcgcagaaaaaaaggatctcaagaagatcctttgatcttttctacggggtctgacgctca
gtggaacgaaaactcacgttaagggattttggtcatgagattatcaaaaaggatcttcacctagatcc
ttttaaattaaaaatgaagttttaaatcaatctaaagtatatatgagtaaacttggtctgacagttac
caatgcttaatcagtgaggcacctatctcagcgatctgtctatttcgttcatccatagttgcctgact
ccccgtcgtgtagataactacgatacgggagggcttaccatctggccccagtgctgcaatgataccgc
gagacccacgctcaccggctccagatttatcagcaataaaccagccagccggaagggccgagcgcaga
agtggtcctgcaactttatccgcctccatccagtctattaattgttgccgggaagctagagtaagtag
ttcgccagttaatagtttgcgcaacgttgttgccattgctacaggcatcgtggtgtcacgctcgtcgt
ttggtatggcttcattcagctccggttcccaacgatcaaggcgagttacatgatcccccatgttgtgc
aaaaaagcggttagctccttcggtcctccgatcgttgtcagaagtaagttggccgcagtgttatcact
catggttatggcagcactgcataattctcttactgtcatgccatccgtaagatgcttttctgtgactg
gtgagtactcaaccaagtcattctgagaatagtgtatgcggcgaccgagttgctcttgcccggcgtca
atacgggataataccgcgccacatagcagaactttaaaagtgctcatcattggaaaacgttcttcggg
gcgaaaactctcaaggatcttaccgctgttgagatccagttcgatgtaacccactcgtgcacccaact
gatcttcagcatcttttactttcaccagcgtttctgggtgagcaaaaacaggaaggcaaaatgccgca
aaaaagggaataagggcgacacggaaatgttgaatactcatactcttcctttttcaatattattgaag
catttatcagggttattgtctcatgagcggatacatatttgaatgtatttagaaaaataaacaaatag
gggttccgcgcacatttccccgaaaagtgccacctgacgtc
pZV01_SARS-CoV-2_UTR-Spike_WT_HexaPro_S1_SPY_catcher003
SEQ ID NO: 83
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccgagaataaactagtattcttctggtccccacagactcagagagaaccc
gccaccatgtttgtgttcctggtgctgctgccactggtgtccagccagtgtgtgaacctgaccaccag
gacccaacttcctcctgcctacaccaactccttcaccaggggagtctactaccctgacaaggtgttca
ggtcctctgtgctgcacagcacccaggacctgttcctgccattcttcagcaatgtgacctggttccat
gccatccatgtgtctggcaccaatggcaccaagaggtttgacaaccctgtgctgccattcaatgatgg
agtctactttgccagcacagagaagagcaacatcatcaggggctggatttttggcaccaccctggaca
gcaagacccagtccctgctgattgtgaacaatgccaccaatgtggtgattaaggtgtgtgagttccag
ttctgtaatgacccattcctgggagtctactaccacaagaacaacaagtcctggatggagtctgagtt
cagggtctactcctctgccaacaactgtacctttgaatatgtgagccaaccattcctgatggacttgg
agggcaagcagggcaacttcaagaacctgagggagtttgtgttcaagaacattgatggctacttcaag
atttacagcaaacacacaccaatcaacctggtgagggacctgccacagggcttctctgccttggaacc
actggtggacctgccaattggcatcaacatcaccaggttccagaccctgctggctctgcacaggtcct
acctgacacctggagactcctcctctggctggacagcaggagcagcagcctactatgtgggctacctc
caaccaaggaccttcctgctgaaatacaatgagaatggcaccatcacagatgctgtggactgtgccct
ggacccactgtctgagaccaagtgtaccctgaaatccttcacagtggagaagggcatctaccagacca
gcaacttcagggtccaaccaacagagagcattgtgaggtttccaaacatcaccaacctgtgtccattt
ggagaggtgttcaatgccaccaggtttgcctctgtctatgcctggaacaggaagaggattagcaactg
tgtggctgactactctgtgctctacaactctgcctccttcagcaccttcaagtgttatggagtgagcc
caaccaaactgaatgacctgtgtttcaccaatgtctatgctgactcctttgtgattaggggagatgag
gtgagacagattgcccctggacaaacaggcaagattgctgactacaactacaaactgcctgatgactt
cacaggctgtgtgattgcctggaacagcaacaacctggacagcaaggtgggaggcaactacaactacc
tctacagactgttcaggaagagcaacctgaaaccatttgagagggacatcagcacagagatttaccag
gctggcagcacaccatgtaatggagtggagggcttcaactgttactttccactccaatcctatggctt
ccaaccaaccaatggagtgggctaccaaccatacagggtggtggtgctgtcctttgaactgctccatg
cccctgccacagtgtgtggaccaaagaagagcaccaacctggtgaagaacaagtgtgtgaacttcaac
ttcaatggactgacaggcacaggagtgctgacagagagcaacaagaagttcctgccattccaacagtt
tggcagggacattgctgacaccacagatgctgtgagggacccacagaccttggagattctggacatca
caccatgttcctttggaggagtgtctgtgattacacctggcaccaacaccagcaaccaggtggctgtg
ctctaccaggatgtgaactgtggcgcgatggtgaccacactgtccggactgtctggagagcagggacc
atccggcgacatgaccacagaggaggattctgccacacacatcaagttcagcaagagggacgaggacg
gaagagagctggcaggagcaaccatggagctgagggatagctccggcaagaccatcagcacatggatc
tccgacggccacgtgaaggatttctacctgtatcccggcaagtacacctttgtggagacagcagcacc
agacggatatgaggtggcaacccctatcgagtttacagtgaacgaggacggacaggtgaccgtggatg
gagaggcaacagagggcgatgcacacacctgactcgagctggtactgcatgcacgcaatgctagctgc
ccctttcccgtcctgggtaccccgagtctcccccgacctcgggtcccaggtatgctcccacctccacc
tgccccactcaccacctctgctagttccagacacctcccaagcacgcagcaatgcagctcaaaacgct
tagcctagccacacccccacgggaaacagcagtgattaacctttagcaataaacgaaagtttaactaa
gctatactaaccccagggttggtcaatttcgtgccagccacaccctggagctagcaaaccgctgatca
gcctcgactgtgccttctagttgccagccatctgttgtttgcccctcccccgtgccttccttgaccct
ggaaggtgccactcccactgtcctttcctaataaaatgaggaaattgcatcgcattgtctgagtaggt
gtcattctattctggggggtggggtggggcaggacagcaagggggaggattgggaagacaatagcagg
catgctggggatgcggtgggctctatggcttctgaggcggaaagaaccagctggggctctagggggta
tccccacgcgccctgtagcggcgcattaagcgcggcgggtgtggtggttacgcgcagcgtgaccgcta
cacttgccagcgccctagcgcccgctcctttcgctttcttcccttcctttctcgccacgttcgccggc
tttccccgtcaagctctaaatcgggggctccctttagggttccgatttagtgctttacggcacctcga
ccccaaaaaacttgattagggtgatggttcacgtagtgggccatcgccctgatagacggtttttcgcc
ctttgacgttggagtccacgttctttaatagtggactcttgttccaaactggaacaacactcaaccct
atctcggtctattcttttgatttataagggattttgccgatttcggcctattggttaaaaaatgagct
gatttaacaaaaatttaacgcgaattaattctgtggaatgtgtgtcagttagggtgtggaaagtcccc
aggctccccagcaggcagaagtatgcaaagcatgcatctcaattagtcagcaaccaggtgtggaaagt
ccccaggctccccagcaggcagaagtatgcaaagcatgcatctcaattagtcagcaaccatagtcccg
cccctaactccgcccatcccgcccctaactccgcccagttccgcccattctccgccccatggctgact
aattttttttatttatgcagaggccgaggccgcctctgcctctgagctattccagaagtagtgaggag
gcttttttggaggcctaggcttttgcaaaaagctcccgggagcttgtatatccattttcggatctgat
caagagacaggatgaggatcgtttcgcatgattgaacaagatggattgcacgcaggttctccggccgc
ttgggtggagaggctattcggctatgactgggcacaacagacaatcggctgctctgatgccgccgtgt
tccggctgtcagcgcaggggcgcccggttctttttgtcaagaccgacctgtccggtgccctgaatgaa
ctgcaggacgaggcagcgcggctatcgtggctggccacgacgggcgttccttgcgcagctgtgctcga
cgttgtcactgaagcgggaagggactggctgctattgggcgaagtgccggggcaggatctcctgtcat
ctcaccttgctcctgccgagaaagtatccatcatggctgatgcaatgcggcggctgcatacgcttgat
ccggctacctgcccattcgaccaccaagcgaaacatcgcatcgagcgagcacgtactcggatggaagc
cggtcttgtcgatcaggatgatctggacgaagagcatcaggggctcgcgccagccgaactgttcgcca
ggctcaaggcgcgcatgcccgacggcgaggatctcgtcgtgacccatggcgatgcctgcttgccgaat
atcatggtggaaaatggccgcttttctggattcatcgactgtggccggctgggtgtggcggaccgcta
tcaggacatagcgttggctacccgtgatattgctgaagagcttggcggcgaatgggctgaccgcttcc
tcgtgctttacggtatcgccgctcccgattcgcagcgcatcgccttctatcgccttcttgacgagttc
ttctgagcgggactctggggttcgaaatgaccgaccaagcgacgcccaacctgccatcacgagatttc
gattccaccgccgccttctatgaaaggttgggcttcggaatcgttttccgggacgccggctggatgat
cctccagcgcggggatctcatgctggagttcttcgcccaccccaacttgtttattgcagcttataatg
gttacaaataaagcaatagcatcacaaatttcacaaataaagcatttttttcactgcattctagttgt
ggtttgtccaaactcatcaatgtatcttatcatgtctgtataccgtcgacctctagctagagcttggc
gtaatcatggtcatagctgtttcctgtgtgaaattgttatccgctcacaattccacacaacatacgag
ccggaagcataaagtgtaaagcctggggtgcctaatgagtgagctaactcacattaattgcgttgcgc
tcactgcccgctttccagtcgggaaacctgtcgtgccagctgcattaatgaatcggccaacgcgcggg
gagaggcggtttgcgtattgggcgctcttccgcttcctcgctcactgactcgctgcgctcggtcgttc
ggctgcggcgagcggtatcagctcactcaaaggcggtaatacggttatccacagaatcaggggataac
gcaggaaagaacatgtgagcaaaaggccagcaaaaggccaggaaccgtaaaaaggccgcgttgctggc
gtttttccataggctccgcccccctgacgagcatcacaaaaatcgacgctcaagtcagaggtggcgaa
acccgacaggactataaagataccaggcgtttccccctggaagctccctcgtgcgctctcctgttccg
accctgccgcttaccggatacctgtccgcctttctcccttcgggaagcgtggcgctttctcatagctc
acgctgtaggtatctcagttcggtgtaggtcgttcgctccaagctgggctgtgtgcacgaaccccccg
ttcagcccgaccgctgcgccttatccggtaactatcgtcttgagtccaacccggtaagacacgactta
tcgccactggcagcagccactggtaacaggattagcagagcgaggtatgtaggcggtgctacagagtt
cttgaagtggtggcctaactacggctacactagaagaacagtatttggtatctgcgctctgctgaagc
cagttaccttcggaaaaagagttggtagctcttgatccggcaaacaaaccaccgctggtagcggtggt
ttttttgtttgcaagcagcagattacgcgcagaaaaaaaggatctcaagaagatcctttgatcttttc
tacggggtctgacgctcagtggaacgaaaactcacgttaagggattttggtcatgagattatcaaaaa
ggatcttcacctagatccttttaaattaaaaatgaagttttaaatcaatctaaagtatatatgagtaa
acttggtctgacagttaccaatgcttaatcagtgaggcacctatctcagcgatctgtctatttcgttc
atccatagttgcctgactccccgtcgtgtagataactacgatacgggagggcttaccatctggcccca
gtgctgcaatgataccgcgagacccacgctcaccggctccagatttatcagcaataaaccagccagcc
ggaagggccgagcgcagaagtggtcctgcaactttatccgcctccatccagtctattaattgttgccg
ggaagctagagtaagtagttcgccagttaatagtttgcgcaacgttgttgccattgctacaggcatcg
tggtgtcacgctcgtcgtttggtatggcttcattcagctccggttcccaacgatcaaggcgagttaca
tgatcccccatgttgtgcaaaaaagcggttagctccttcggtcctccgatcgttgtcagaagtaagtt
ggccgcagtgttatcactcatggttatggcagcactgcataattctcttactgtcatgccatccgtaa
gatgcttttctgtgactggtgagtactcaaccaagtcattctgagaatagtgtatgcggcgaccgagt
tgctcttgcccggcgtcaatacgggataataccgcgccacatagcagaactttaaaagtgctcatcat
tggaaaacgttcttcggggcgaaaactctcaaggatcttaccgctgttgagatccagttcgatgtaac
ccactcgtgcacccaactgatcttcagcatcttttactttcaccagcgtttctgggtgagcaaaaaca
ggaaggcaaaatgccgcaaaaaagggaataagggcgacacggaaatgttgaatactcatactcttcct
ttttcaatattattgaagcatttatcagggttattgtctcatgagcggatacatatttgaatgtattt
agaaaaataaacaaataggggttccgcgcacatttccccgaaaagtgccacctgacgtc
pZV02_SARS-CoV-2_UTR-Spike_WT_hexapro_SPY_tag003_S2
SEQ ID NO: 84
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccgagaataaactagtattcttctggtccccacagactcagagagaaccc
gccaccatgggccgtggcgtgcctcatatcgtgatggtggacgcctacaagcgttacaagactgaggt
gcctgtggctatccatgctgaccaacttacaccaacctggagggtctacagcacaggcagcaatgtgt
tccagaccagggctggctgtctgattggagcagagcatgtgaacaactcctatgagtgtgacatccca
attggagcaggcatctgtgcctcctaccagacccagaccaacagcccaggctctgcatcttctgtggc
aagccagagcatcattgcctacacaatgagtctgggagcagagaactctgtggcttacagcaacaaca
gcattgccatcccaaccaacttcaccatctctgtgaccacagagattctgcctgtgagtatgaccaag
acctctgtggactgtacaatgtatatctgtggagacagcacagagtgtagcaacctgctgctccaata
tggctccttctgtacccaacttaacagggctctgacaggcattgctgtggaacaggacaagaacaccc
aggaggtgtttgcccaggtgaagcagatttacaagacacctccaatcaaggactttggaggcttcaac
ttcagccagattctgcctgacccaagcaagccaagcaagaggtcccctattgaggacctgctgttcaa
caaggtgaccctggctgatgctggcttcatcaagcaatatggagactgtctgggagacattgctgcca
gggacctgatttgtgcccagaagttcaatggactgacagtgctgcctccactgctgacagatgagatg
attgcccaatacacctctgccctgctggctggcaccatcacctctggctggacctttggagcaggacc
agccctccaaatcccatttccaatgcagatggcttacaggttcaatggcattggagtgacccagaatg
tgctctatgagaaccagaaactgattgccaaccagttcaactctgccattggcaagattcaggactcc
ctgtccagcacaccatctgccctgggcaaactccaagatgtggtgaaccagaatgcccaggctctgaa
caccctggtgaagcaactttccagcaactttggagccatctcctctgtgctgaatgacatcctgagca
gactggacccaccagaggctgaggtccagattgacagactgattacaggcagactccaatccctccaa
acctatgtgacccaacaacttatcagggctgctgagattagggcatctgccaacctggctgccaccaa
gatgagtgagtgtgtgctgggacaaagcaagagggtggacttctgtggcaagggctaccacctgatga
gttttccacagtctgcccctcatggagtggtgttcctgcatgtgacctatgtgcctgcccaggagaag
aacttcaccacagcccctgccatctgccatgatggcaaggctcactttccaagggagggagtgtttgt
gagcaatggcacccactggtttgtgacccagaggaacttctatgaaccacagattatcaccacagaca
acacctttgtgtctggcaactgtgatgtggtgattggcattgtgaacaacacagtctatgacccactc
caacctgaactggactccttcaaggaggaactggacaaatacttcaagaaccacaccagccctgatgt
ggacctgggagacatctctggcatcaatgcctctgtggtgaacatccagaaggagattgacagactga
atgaggtggctaagaacctgaatgagtccctgattgacctccaagaactgggcaaatatgaacaatac
atcaagtggccatggtacatctggctgggcttcattgctggactgattgccattgtgatggtgaccat
aatgctgtgttgtatgacctcctgttgttcctgtctgaaaggctgttgttcctgtggctcctgttgta
agtttgatgaggatgactctgaacctgtgctgaaaggagtgaaactgcactacacctgactcgagctg
gtactgcatgcacgcaatgctagctgcccctttcccgtcctgggtaccccgagtctcccccgacctcg
ggtcccaggtatgctcccacctccacctgccccactcaccacctctgctagttccagacacctcccaa
gcacgcagcaatgcagctcaaaacgcttagcctagccacacccccacgggaaacagcagtgattaacc
tttagcaataaacgaaagtttaactaagctatactaaccccagggttggtcaatttcgtgccagccac
accctggagctagcaaaccgctgatcagcctcgactgtgccttctagttgccagccatctgttgtttg
cccctcccccgtgccttccttgaccctggaaggtgccactcccactgtcctttcctaataaaatgagg
aaattgcatcgcattgtctgagtaggtgtcattctattctggggggtggggtggggcaggacagcaag
ggggaggattgggaagacaatagcaggcatgctggggatgcggtgggctctatggcttctgaggcgga
aagaaccagctggggctctagggggtatccccacgcgccctgtagcggcgcattaagcgcggcgggtg
tggtggttacgcgcagcgtgaccgctacacttgccagcgccctagcgcccgctcctttcgctttcttc
ccttcctttctcgccacgttcgccggctttccccgtcaagctctaaatcgggggctccctttagggtt
ccgatttagtgctttacggcacctcgaccccaaaaaacttgattagggtgatggttcacgtagtgggc
catcgccctgatagacggtttttcgccctttgacgttggagtccacgttctttaatagtggactcttg
ttccaaactggaacaacactcaaccctatctcggtctattcttttgatttataagggattttgccgat
ttcggcctattggttaaaaaatgagctgatttaacaaaaatttaacgcgaattaattctgtggaatgt
gtgtcagttagggtgtggaaagtccccaggctccccagcaggcagaagtatgcaaagcatgcatctca
attagtcagcaaccaggtgtggaaagtccccaggctccccagcaggcagaagtatgcaaagcatgcat
ctcaattagtcagcaaccatagtcccgcccctaactccgcccatcccgcccctaactccgcccagttc
cgcccattctccgccccatggctgactaattttttttatttatgcagaggccgaggccgcctctgcct
ctgagctattccagaagtagtgaggaggcttttttggaggcctaggcttttgcaaaaagctcccggga
gcttgtatatccattttcggatctgatcaagagacaggatgaggatcgtttcgcatgattgaacaaga
tggattgcacgcaggttctccggccgcttgggtggagaggctattcggctatgactgggcacaacaga
caatcggctgctctgatgccgccgtgttccggctgtcagcgcaggggcgcccggttctttttgtcaag
accgacctgtccggtgccctgaatgaactgcaggacgaggcagcgcggctatcgtggctggccacgac
gggcgttccttgcgcagctgtgctcgacgttgtcactgaagcgggaagggactggctgctattgggcg
aagtgccggggcaggatctcctgtcatctcaccttgctcctgccgagaaagtatccatcatggctgat
gcaatgcggcggctgcatacgcttgatccggctacctgcccattcgaccaccaagcgaaacatcgcat
cgagcgagcacgtactcggatggaagccggtcttgtcgatcaggatgatctggacgaagagcatcagg
ggctcgcgccagccgaactgttcgccaggctcaaggcgcgcatgcccgacggcgaggatctcgtcgtg
acccatggcgatgcctgcttgccgaatatcatggtggaaaatggccgcttttctggattcatcgactg
tggccggctgggtgtggcggaccgctatcaggacatagcgttggctacccgtgatattgctgaagagc
ttggcggcgaatgggctgaccgcttcctcgtgctttacggtatcgccgctcccgattcgcagcgcatc
gccttctatcgccttcttgacgagttcttctgagcgggactctggggttcgaaatgaccgaccaagcg
acgcccaacctgccatcacgagatttcgattccaccgccgccttctatgaaaggttgggcttcggaat
cgttttccgggacgccggctggatgatcctccagcgcggggatctcatgctggagttcttcgcccacc
ccaacttgtttattgcagcttataatggttacaaataaagcaatagcatcacaaatttcacaaataaa
gcatttttttcactgcattctagttgtggtttgtccaaactcatcaatgtatcttatcatgtctgtat
accgtcgacctctagctagagcttggcgtaatcatggtcatagctgtttcctgtgtgaaattgttatc
cgctcacaattccacacaacatacgagccggaagcataaagtgtaaagcctggggtgcctaatgagtg
agctaactcacattaattgcgttgcgctcactgcccgctttccagtcgggaaacctgtcgtgccagct
gcattaatgaatcggccaacgcgcggggagaggcggtttgcgtattgggcgctcttccgcttcctcgc
tcactgactcgctgcgctcggtcgttcggctgcggcgagcggtatcagctcactcaaaggcggtaata
cggttatccacagaatcaggggataacgcaggaaagaacatgtgagcaaaaggccagcaaaaggccag
gaaccgtaaaaaggccgcgttgctggcgtttttccataggctccgcccccctgacgagcatcacaaaa
atcgacgctcaagtcagaggtggcgaaacccgacaggactataaagataccaggcgtttccccctgga
agctccctcgtgcgctctcctgttccgaccctgccgcttaccggatacctgtccgcctttctcccttc
gggaagcgtggcgctttctcatagctcacgctgtaggtatctcagttcggtgtaggtcgttcgctcca
agctgggctgtgtgcacgaaccccccgttcagcccgaccgctgcgccttatccggtaactatcgtctt
gagtccaacccggtaagacacgacttatcgccactggcagcagccactggtaacaggattagcagagc
gaggtatgtaggcggtgctacagagttcttgaagtggtggcctaactacggctacactagaagaacag
tatttggtatctgcgctctgctgaagccagttaccttcggaaaaagagttggtagctcttgatccggc
aaacaaaccaccgctggtagcggtggtttttttgtttgcaagcagcagattacgcgcagaaaaaaagg
atctcaagaagatcctttgatcttttctacggggtctgacgctcagtggaacgaaaactcacgttaag
ggattttggtcatgagattatcaaaaaggatcttcacctagatccttttaaattaaaaatgaagtttt
aaatcaatctaaagtatatatgagtaaacttggtctgacagttaccaatgcttaatcagtgaggcacc
tatctcagcgatctgtctatttcgttcatccatagttgcctgactccccgtcgtgtagataactacga
tacgggagggcttaccatctggccccagtgctgcaatgataccgcgagacccacgctcaccggctcca
gatttatcagcaataaaccagccagccggaagggccgagcgcagaagtggtcctgcaactttatccgc
ctccatccagtctattaattgttgccgggaagctagagtaagtagttcgccagttaatagtttgcgca
acgttgttgccattgctacaggcatcgtggtgtcacgctcgtcgtttggtatggcttcattcagctcc
ggttcccaacgatcaaggcgagttacatgatcccccatgttgtgcaaaaaagcggttagctccttcgg
tcctccgatcgttgtcagaagtaagttggccgcagtgttatcactcatggttatggcagcactgcata
attctcttactgtcatgccatccgtaagatgcttttctgtgactggtgagtactcaaccaagtcattc
tgagaatagtgtatgcggcgaccgagttgctcttgcccggcgtcaatacgggataataccgcgccaca
tagcagaactttaaaagtgctcatcattggaaaacgttcttcggggcgaaaactctcaaggatcttac
cgctgttgagatccagttcgatgtaacccactcgtgcacccaactgatcttcagcatcttttactttc
accagcgtttctgggtgagcaaaaacaggaaggcaaaatgccgcaaaaaagggaataagggcgacacg
gaaatgttgaatactcatactcttcctttttcaatattattgaagcatttatcagggttattgtctca
tgagcggatacatatttgaatgtatttagaaaaataaacaaataggggttccgcgcacatttccccga
aaagtgccacctgacgtc
pZV03_SARS-CoV-2-UTR-spike_delta_HexaPro_S1_S683_furin_SPY_tag
SEQ ID NO: 85
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccgagaataaactagtattcttctggtccccacagactcagagagaaccc
gccaccatgtttgtgttcctggtgctgctgccactggtgtccagccagtgtgtgaacctgaggaccag
gacccaacttcctcctgcctacaccaactccttcaccaggggagtctactaccctgacaaggtgttca
ggtcctctgtgctgcacagcacccaggacctgttcctgccattcttcagcaatgtgacctggttccat
gccatccatgtgtctggcaccaatggcaccaagaggtttgacaaccctgtgctgccattcaatgatgg
agtctactttgccagcacagagaagagcaacatcatcaggggctggatttttggcaccaccctggaca
gcaagacccagtccctgctgattgtgaacaatgccaccaatgtggtgattaaggtgtgtgagttccag
ttctgtaatgacccattcctggacgtctactaccacaagaacaacaagtcctggatggagtctggcgt
ctactcctctgccaacaactgtacctttgaatatgtgagccaaccattcctgatggacttggagggca
agcagggcaacttcaagaacctgagggagtttgtgttcaagaacattgatggctacttcaagatttac
agcaaacacacaccaatcaacctggtgagggacctgccacagggcttctctgccttggaaccactggt
ggacctgccaattggcatcaacatcaccaggttccagaccctgctggctctgcacaggtcctacctga
cacctggagactcctcctctggctggacagcaggagcagcagcctactatgtgggctacctccaacca
aggaccttcctgctgaaatacaatgagaatggcaccatcacagatgctgtggactgtgccctggaccc
actgtctgagaccaagtgtaccctgaaatccttcacagtggagaagggcatctaccagaccagcaact
tcagggtccaaccaacagagagcattgtgaggtttccaaacatcaccaacctgtgtccatttggagag
gtgttcaatgccaccaggtttgcctctgtctatgcctggaacaggaagaggattagcaactgtgtggc
tgactactctgtgctctacaactctgcctccttcagcaccttcaagtgttatggagtgagcccaacca
aactgaatgacctgtgtttcaccaatgtctatgctgactcctttgtgattaggggagatgaggtgaga
cagattgcccctggacaaacaggcaagattgctgactacaactacaaactgcctgatgacttcacagg
ctgtgtgattgcctggaacagcaacaacctggacagcaaggtgggaggcaactacaactacaggtaca
gactgttcaggaagagcaacctgaaaccatttgagagggacatcagcacagagatttaccaggctggc
agcaagccatgtaatggagtggagggcttcaactgttactttccactccaatcctatggcttccaacc
aaccaatggagtgggctaccaaccatacagggtggtggtgctgtcctttgaactgctccatgcccctg
ccacagtgtgtggaccaaagaagagcaccaacctggtgaagaacaagtgtgtgaacttcaacttcaat
ggactgacaggcacaggagtgctgacagagagcaacaagaagttcctgccattccaacagtttggcag
ggacattgctgacaccacagatgctgtgagggacccacagaccttggagattctggacatcacaccat
gttcctttggaggagtgtctgtgattacacctggcaccaacaccagcaaccaggtggctgtgctctac
cagggcgtgaactgtactgaggtgcctgtggctatccatgctgaccaacttacaccaacctggagggt
ctacagcacaggaagcaacgtgttccagaccagggctggctgtctgattggagcagagcatgtgaaca
actcctatgagtgtgacatcccaattggagcaggcatctgtgcctcctaccagacccagaccaacagc
aggggctctgcatctcgtggcgtgcctcatatcgtgatggtggacgcctacaagcgttacaagtgact
cgagctggtactgcatgcacgcaatgctagctgcccctttcccgtcctgggtaccccgagtctccccc
gacctcgggtcccaggtatgctcccacctccacctgccccactcaccacctctgctagttccagacac
ctcccaagcacgcagcaatgcagctcaaaacgcttagcctagccacacccccacgggaaacagcagtg
attaacctttagcaataaacgaaagtttaactaagctatactaaccccagggttggtcaatttcgtgc
cagccacaccctggagctagcaaaccgctgatcagcctcgactgtgccttctagttgccagccatctg
ttgtttgcccctcccccgtgccttccttgaccctggaaggtgccactcccactgtcctttcctaataa
aatgaggaaattgcatcgcattgtctgagtaggtgtcattctattctggggggtggggtggggcagga
cagcaagggggaggattgggaagacaatagcaggcatgctggggatgcggtgggctctatggcttctg
aggcggaaagaaccagctggggctctagggggtatccccacgcgccctgtagcggcgcattaagcgcg
gcgggtgtggtggttacgcgcagcgtgaccgctacacttgccagcgccctagcgcccgctcctttcgc
tttcttcccttcctttctcgccacgttcgccggctttccccgtcaagctctaaatcgggggctccctt
tagggttccgatttagtgctttacggcacctcgaccccaaaaaacttgattagggtgatggttcacgt
agtgggccatcgccctgatagacggtttttcgccctttgacgttggagtccacgttctttaatagtgg
actcttgttccaaactggaacaacactcaaccctatctcggtctattcttttgatttataagggattt
tgccgatttcggcctattggttaaaaaatgagctgatttaacaaaaatttaacgcgaattaattctgt
ggaatgtgtgtcagttagggtgtggaaagtccccaggctccccagcaggcagaagtatgcaaagcatg
catctcaattagtcagcaaccaggtgtggaaagtccccaggctccccagcaggcagaagtatgcaaag
catgcatctcaattagtcagcaaccatagtcccgcccctaactccgcccatcccgcccctaactccgc
ccagttccgcccattctccgccccatggctgactaattttttttatttatgcagaggccgaggccgcc
tctgcctctgagctattccagaagtagtgaggaggcttttttggaggcctaggcttttgcaaaaagct
cccgggagcttgtatatccattttcggatctgatcaagagacaggatgaggatcgtttcgcatgattg
aacaagatggattgcacgcaggttctccggccgcttgggtggagaggctattcggctatgactgggca
caacagacaatcggctgctctgatgccgccgtgttccggctgtcagcgcaggggcgcccggttctttt
tgtcaagaccgacctgtccggtgccctgaatgaactgcaggacgaggcagcgcggctatcgtggctgg
ccacgacgggcgttccttgcgcagctgtgctcgacgttgtcactgaagcgggaagggactggctgcta
ttgggcgaagtgccggggcaggatctcctgtcatctcaccttgctcctgccgagaaagtatccatcat
ggctgatgcaatgcggcggctgcatacgcttgatccggctacctgcccattcgaccaccaagcgaaac
atcgcatcgagcgagcacgtactcggatggaagccggtcttgtcgatcaggatgatctggacgaagag
catcaggggctcgcgccagccgaactgttcgccaggctcaaggcgcgcatgcccgacggcgaggatct
cgtcgtgacccatggcgatgcctgcttgccgaatatcatggtggaaaatggccgcttttctggattca
tcgactgtggccggctgggtgtggcggaccgctatcaggacatagcgttggctacccgtgatattgct
gaagagcttggcggcgaatgggctgaccgcttcctcgtgctttacggtatcgccgctcccgattcgca
gcgcatcgccttctatcgccttcttgacgagttcttctgagcgggactctggggttcgaaatgaccga
ccaagcgacgcccaacctgccatcacgagatttcgattccaccgccgccttctatgaaaggttgggct
tcggaatcgttttccgggacgccggctggatgatcctccagcgcggggatctcatgctggagttcttc
gcccaccccaacttgtttattgcagcttataatggttacaaataaagcaatagcatcacaaatttcac
aaataaagcatttttttcactgcattctagttgtggtttgtccaaactcatcaatgtatcttatcatg
tctgtataccgtcgacctctagctagagcttggcgtaatcatggtcatagctgtttcctgtgtgaaat
tgttatccgctcacaattccacacaacatacgagccggaagcataaagtgtaaagcctggggtgccta
atgagtgagctaactcacattaattgcgttgcgctcactgcccgctttccagtcgggaaacctgtcgt
gccagctgcattaatgaatcggccaacgcgcggggagaggcggtttgcgtattgggcgctcttccgct
tcctcgctcactgactcgctgcgctcggtcgttcggctgcggcgagcggtatcagctcactcaaaggc
ggtaatacggttatccacagaatcaggggataacgcaggaaagaacatgtgagcaaaaggccagcaaa
aggccaggaaccgtaaaaaggccgcgttgctggcgtttttccataggctccgcccccctgacgagcat
cacaaaaatcgacgctcaagtcagaggtggcgaaacccgacaggactataaagataccaggcgtttcc
ccctggaagctccctcgtgcgctctcctgttccgaccctgccgcttaccggatacctgtccgcctttc
tcccttcgggaagcgtggcgctttctcatagctcacgctgtaggtatctcagttcggtgtaggtcgtt
cgctccaagctgggctgtgtgcacgaaccccccgttcagcccgaccgctgcgccttatccggtaacta
tcgtcttgagtccaacccggtaagacacgacttatcgccactggcagcagccactggtaacaggatta
gcagagcgaggtatgtaggcggtgctacagagttcttgaagtggtggcctaactacggctacactaga
agaacagtatttggtatctgcgctctgctgaagccagttaccttcggaaaaagagttggtagctcttg
atccggcaaacaaaccaccgctggtagcggtggtttttttgtttgcaagcagcagattacgcgcagaa
aaaaaggatctcaagaagatcctttgatcttttctacggggtctgacgctcagtggaacgaaaactca
cgttaagggattttggtcatgagattatcaaaaaggatcttcacctagatccttttaaattaaaaatg
aagttttaaatcaatctaaagtatatatgagtaaacttggtctgacagttaccaatgcttaatcagtg
aggcacctatctcagcgatctgtctatttcgttcatccatagttgcctgactccccgtcgtgtagata
actacgatacgggagggcttaccatctggccccagtgctgcaatgataccgcgagacccacgctcacc
ggctccagatttatcagcaataaaccagccagccggaagggccgagcgcagaagtggtcctgcaactt
tatccgcctccatccagtctattaattgttgccgggaagctagagtaagtagttcgccagttaatagt
ttgcgcaacgttgttgccattgctacaggcatcgtggtgtcacgctcgtcgtttggtatggcttcatt
cagctccggttcccaacgatcaaggcgagttacatgatcccccatgttgtgcaaaaaagcggttagct
ccttcggtcctccgatcgttgtcagaagtaagttggccgcagtgttatcactcatggttatggcagca
ctgcataattctcttactgtcatgccatccgtaagatgcttttctgtgactggtgagtactcaaccaa
gtcattctgagaatagtgtatgcggcgaccgagttgctcttgcccggcgtcaatacgggataataccg
cgccacatagcagaactttaaaagtgctcatcattggaaaacgttcttcggggcgaaaactctcaagg
atcttaccgctgttgagatccagttcgatgtaacccactcgtgcacccaactgatcttcagcatcttt
tactttcaccagcgtttctgggtgagcaaaaacaggaaggcaaaatgccgcaaaaaagggaataaggg
cgacacggaaatgttgaatactcatactcttcctttttcaatattattgaagcatttatcagggttat
tgtctcatgagcggatacatatttgaatgtatttagaaaaataaacaaataggggttccgcgcacatt
tccccgaaaagtgccacctgacgtc
pZV04_SARS-CoV-2-UTR-spike_delta_HexaPro_SPY_catcher_S684_S2
SEQ ID NO: 86
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccgagaataaactagtattcttctggtccccacagactcagagagaaccc
gccaccatgtttgtgttcctggtgctgctgccactggtgtccagcgtgaccacactgtccggactgtc
tggagagcagggaccatccggcgacatgaccacagaggaggattctgccacacacatcaagttcagca
agagggacgaggacggaagagagctggcaggagcaaccatggagctgagggatagctccggcaagacc
atcagcacatggatctccgacggccacgtgaaggatttctacctgtatcccggcaagtacacctttgt
ggagacagcagcaccagacggatatgaggtggcaacccctatcgagtttacagtgaacgaggacggac
aggtgaccgtggatggagaggcaacagagggcgatgcacacaccggcagcagctctgtggcaagccag
agcatcattgcctacacaatgagtctgggagcagagaactctgtggcttacagcaacaacagcattgc
catcccaaccaacttcaccatctctgtgaccacagagattctgcctgtgagtatgaccaagacctctg
tggactgtacaatgtatatctgtggagacagcacagagtgtagcaacctgctgctccaatatggctcc
ttctgtacccaacttaacagggctctgacaggcattgctgtggaacaggacaagaacacccaggaggt
gtttgcccaggtgaagcagatttacaagacacctccaatcaaggactttggaggcttcaacttcagcc
agattctgcctgacccaagcaagccaagcaagaggtcccctattgaggacctgctgttcaacaaggtg
accctggctgatgctggcttcatcaagcaatatggagactgtctgggagacattgccgccagggacct
gatttgtgcccagaagttcaatggactgacagtgctgcctccactgctgacagatgagatgattgccc
aatacacctctgccctgctggctggcaccatcacctctggctggacctttggagcaggaccagccctc
caaatcccatttccaatgcagatggcttacaggttcaatggcattggagtgacccagaatgtgctcta
tgagaaccagaaactgattgccaaccagttcaactctgccattggcaagattcaggactccctgtcca
gcacaccatctgccctgggcaaactccaaaacgtggtgaaccagaatgcccaggctctgaacaccctg
gtgaagcaactttccagcaactttggagccatctcctctgtgctgaatgacatcctgagcagactgga
cccaccagaggctgaggtccagattgacagactgattacaggcagactccaatccctccaaacctatg
tgacccaacaacttatcagggctgctgagattagggcatctgccaacctggctgccaccaagatgagt
gagtgtgtgctgggacaaagcaagagggtggacttctgtggcaagggctaccacctgatgagttttcc
acagtctgcccctcatggagtggtgttcctgcatgtgacctatgtgcctgcccaggagaagaacttca
ccacagcccctgccatctgccatgatggcaaggctcactttccaagggagggagtgtttgtgagcaat
ggcacccactggtttgtgacccagaggaacttctatgaaccacagattatcaccacagacaacacctt
tgtgtctggcaactgtgatgtggtgattggcattgtgaacaacacagtctatgacccactccaacctg
aactggactccttcaaggaggaactggacaaatacttcaagaaccacaccagccctgatgtggacctg
ggagacatctctggcatcaatgcctctgtggtgaacatccagaaggagattgacagactgaatgaggt
ggctaagaacctgaatgagtccctgattgacctccaagaactgggcaaatatgaacaatacatcaagt
ggccatggtacatctggctgggcttcattgctggactgattgccattgtgatggtgaccataatgctg
tgttgtatgacctcctgttgttcctgtctgaaaggctgttgttcctgtggctcctgttgtaagtttga
tgaggatgactctgaacctgtgctgaaaggagtgaaactgcactacacctgactcgagctggtactgc
atgcacgcaatgctagctgcccctttcccgtcctgggtaccccgagtctcccccgacctcgggtccca
ggtatgctcccacctccacctgccccactcaccacctctgctagttccagacacctcccaagcacgca
gcaatgcagctcaaaacgcttagcctagccacacccccacgggaaacagcagtgattaacctttagca
ataaacgaaagtttaactaagctatactaaccccagggttggtcaatttcgtgccagccacaccctgg
agctagcaaaccgctgatcagcctcgactgtgccttctagttgccagccatctgttgtttgcccctcc
cccgtgccttccttgaccctggaaggtgccactcccactgtcctttcctaataaaatgaggaaattgc
atcgcattgtctgagtaggtgtcattctattctggggggtggggtggggcaggacagcaagggggagg
attgggaagacaatagcaggcatgctggggatgcggtgggctctatggcttctgaggcggaaagaacc
agctggggctctagggggtatccccacgcgccctgtagcggcgcattaagcgcggcgggtgtggtggt
tacgcgcagcgtgaccgctacacttgccagcgccctagcgcccgctcctttcgctttcttcccttcct
ttctcgccacgttcgccggctttccccgtcaagctctaaatcgggggctccctttagggttccgattt
agtgctttacggcacctcgaccccaaaaaacttgattagggtgatggttcacgtagtgggccatcgcc
ctgatagacggtttttcgccctttgacgttggagtccacgttctttaatagtggactcttgttccaaa
ctggaacaacactcaaccctatctcggtctattcttttgatttataagggattttgccgatttcggcc
tattggttaaaaaatgagctgatttaacaaaaatttaacgcgaattaattctgtggaatgtgtgtcag
ttagggtgtggaaagtccccaggctccccagcaggcagaagtatgcaaagcatgcatctcaattagtc
agcaaccaggtgtggaaagtccccaggctccccagcaggcagaagtatgcaaagcatgcatctcaatt
agtcagcaaccatagtcccgcccctaactccgcccatcccgcccctaactccgcccagttccgcccat
tctccgccccatggctgactaattttttttatttatgcagaggccgaggccgcctctgcctctgagct
attccagaagtagtgaggaggcttttttggaggcctaggcttttgcaaaaagctcccgggagcttgta
tatccattttcggatctgatcaagagacaggatgaggatcgtttcgcatgattgaacaagatggattg
cacgcaggttctccggccgcttgggtggagaggctattcggctatgactgggcacaacagacaatcgg
ctgctctgatgccgccgtgttccggctgtcagcgcaggggcgcccggttctttttgtcaagaccgacc
tgtccggtgccctgaatgaactgcaggacgaggcagcgcggctatcgtggctggccacgacgggcgtt
ccttgcgcagctgtgctcgacgttgtcactgaagcgggaagggactggctgctattgggcgaagtgcc
ggggcaggatctcctgtcatctcaccttgctcctgccgagaaagtatccatcatggctgatgcaatgc
ggcggctgcatacgcttgatccggctacctgcccattcgaccaccaagcgaaacatcgcatcgagcga
gcacgtactcggatggaagccggtcttgtcgatcaggatgatctggacgaagagcatcaggggctcgc
gccagccgaactgttcgccaggctcaaggcgcgcatgcccgacggcgaggatctcgtcgtgacccatg
gcgatgcctgcttgccgaatatcatggtggaaaatggccgcttttctggattcatcgactgtggccgg
ctgggtgtggcggaccgctatcaggacatagcgttggctacccgtgatattgctgaagagcttggcgg
cgaatgggctgaccgcttcctcgtgctttacggtatcgccgctcccgattcgcagcgcatcgccttct
atcgccttcttgacgagttcttctgagcgggactctggggttcgaaatgaccgaccaagcgacgccca
acctgccatcacgagatttcgattccaccgccgccttctatgaaaggttgggcttcggaatcgttttc
cgggacgccggctggatgatcctccagcgcggggatctcatgctggagttcttcgcccaccccaactt
gtttattgcagcttataatggttacaaataaagcaatagcatcacaaatttcacaaataaagcatttt
tttcactgcattctagttgtggtttgtccaaactcatcaatgtatcttatcatgtctgtataccgtcg
acctctagctagagcttggcgtaatcatggtcatagctgtttcctgtgtgaaattgttatccgctcac
aattccacacaacatacgagccggaagcataaagtgtaaagcctggggtgcctaatgagtgagctaac
tcacattaattgcgttgcgctcactgcccgctttccagtcgggaaacctgtcgtgccagctgcattaa
tgaatcggccaacgcgcggggagaggcggtttgcgtattgggcgctcttccgcttcctcgctcactga
ctcgctgcgctcggtcgttcggctgcggcgagcggtatcagctcactcaaaggcggtaatacggttat
ccacagaatcaggggataacgcaggaaagaacatgtgagcaaaaggccagcaaaaggccaggaaccgt
aaaaaggccgcgttgctggcgtttttccataggctccgcccccctgacgagcatcacaaaaatcgacg
ctcaagtcagaggtggcgaaacccgacaggactataaagataccaggcgtttccccctggaagctccc
tcgtgcgctctcctgttccgaccctgccgcttaccggatacctgtccgcctttctcccttcgggaagc
gtggcgctttctcatagctcacgctgtaggtatctcagttcggtgtaggtcgttcgctccaagctggg
ctgtgtgcacgaaccccccgttcagcccgaccgctgcgccttatccggtaactatcgtcttgagtcca
acccggtaagacacgacttatcgccactggcagcagccactggtaacaggattagcagagcgaggtat
gtaggcggtgctacagagttcttgaagtggtggcctaactacggctacactagaagaacagtatttgg
tatctgcgctctgctgaagccagttaccttcggaaaaagagttggtagctcttgatccggcaaacaaa
ccaccgctggtagcggtggtttttttgtttgcaagcagcagattacgcgcagaaaaaaaggatctcaa
gaagatcctttgatcttttctacggggtctgacgctcagtggaacgaaaactcacgttaagggatttt
ggtcatgagattatcaaaaaggatcttcacctagatccttttaaattaaaaatgaagttttaaatcaa
tctaaagtatatatgagtaaacttggtctgacagttaccaatgcttaatcagtgaggcacctatctca
gcgatctgtctatttcgttcatccatagttgcctgactccccgtcgtgtagataactacgatacggga
gggcttaccatctggccccagtgctgcaatgataccgcgagacccacgctcaccggctccagatttat
cagcaataaaccagccagccggaagggccgagcgcagaagtggtcctgcaactttatccgcctccatc
cagtctattaattgttgccgggaagctagagtaagtagttcgccagttaatagtttgcgcaacgttgt
tgccattgctacaggcatcgtggtgtcacgctcgtcgtttggtatggcttcattcagctccggttccc
aacgatcaaggcgagttacatgatcccccatgttgtgcaaaaaagcggttagctccttcggtcctccg
atcgttgtcagaagtaagttggccgcagtgttatcactcatggttatggcagcactgcataattctct
tactgtcatgccatccgtaagatgcttttctgtgactggtgagtactcaaccaagtcattctgagaat
agtgtatgcggcgaccgagttgctcttgcccggcgtcaatacgggataataccgcgccacatagcaga
actttaaaagtgctcatcattggaaaacgttcttcggggcgaaaactctcaaggatcttaccgctgtt
gagatccagttcgatgtaacccactcgtgcacccaactgatcttcagcatcttttactttcaccagcg
tttctgggtgagcaaaaacaggaaggcaaaatgccgcaaaaaagggaataagggcgacacggaaatgt
tgaatactcatactcttcctttttcaatattattgaagcatttatcagggttattgtctcatgagcgg
atacatatttgaatgtatttagaaaaataaacaaataggggttccgcgcacatttccccgaaaagtgc
cacctgacgtc
pZV05_SARS-CoV-2-UTR-spike_delta_HexaPro_S1_G637_SPY_tag
SEQ ID NO: 87
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccgagaataaactagtattcttctggtccccacagactcagagagaaccc
gccaccatgtttgtgttcctggtgctgctgccactggtgtccagccagtgtgtgaacctgaggaccag
gacccaacttcctcctgcctacaccaactccttcaccaggggagtctactaccctgacaaggtgttca
ggtcctctgtgctgcacagcacccaggacctgttcctgccattcttcagcaatgtgacctggttccat
gccatccatgtgtctggcaccaatggcaccaagaggtttgacaaccctgtgctgccattcaatgatgg
agtctactttgccagcacagagaagagcaacatcatcaggggctggatttttggcaccaccctggaca
gcaagacccagtccctgctgattgtgaacaatgccaccaatgtggtgattaaggtgtgtgagttccag
ttctgtaatgacccattcctggacgtctactaccacaagaacaacaagtcctggatggagtctggcgt
ctactcctctgccaacaactgtacctttgaatatgtgagccaaccattcctgatggacttggagggca
agcagggcaacttcaagaacctgagggagtttgtgttcaagaacattgatggctacttcaagatttac
agcaaacacacaccaatcaacctggtgagggacctgccacagggcttctctgccttggaaccactggt
ggacctgccaattggcatcaacatcaccaggttccagaccctgctggctctgcacaggtcctacctga
cacctggagactcctcctctggctggacagcaggagcagcagcctactatgtgggctacctccaacca
aggaccttcctgctgaaatacaatgagaatggcaccatcacagatgctgtggactgtgccctggaccc
actgtctgagaccaagtgtaccctgaaatccttcacagtggagaagggcatctaccagaccagcaact
tcagggtccaaccaacagagagcattgtgaggtttccaaacatcaccaacctgtgtccatttggagag
gtgttcaatgccaccaggtttgcctctgtctatgcctggaacaggaagaggattagcaactgtgtggc
tgactactctgtgctctacaactctgcctccttcagcaccttcaagtgttatggagtgagcccaacca
aactgaatgacctgtgtttcaccaatgtctatgctgactcctttgtgattaggggagatgaggtgaga
cagattgcccctggacaaacaggcaagattgctgactacaactacaaactgcctgatgacttcacagg
ctgtgtgattgcctggaacagcaacaacctggacagcaaggtgggaggcaactacaactacaggtaca
gactgttcaggaagagcaacctgaaaccatttgagagggacatcagcacagagatttaccaggctggc
agcaagccatgtaatggagtggagggcttcaactgttactttccactccaatcctatggcttccaacc
aaccaatggagtgggctaccaaccatacagggtggtggtgctgtcctttgaactgctccatgcccctg
ccacagtgtgtggaccaaagaagagcaccaacctggtgaagaacaagtgtgtgaacttcaacttcaat
ggactgacaggcacaggagtgctgacagagagcaacaagaagttcctgccattccaacagtttggcag
ggacattgctgacaccacagatgctgtgagggacccacagaccttggagattctggacatcacaccat
gttcctttggaggagtgtctgtgattacacctggcaccaacaccagcaaccaggtggctgtgctctac
cagggcgtgaactgtactgaggtgcctgtggctatccatgctgaccaacttacaccaacctggagggt
ctacagcacaggacgtggcgtgcctcatatcgtgatggtggacgcctacaagcgttacaagtgactcg
agctggtactgcatgcacgcaatgctagctgcccctttcccgtcctgggtaccccgagtctcccccga
cctcgggtcccaggtatgctcccacctccacctgccccactcaccacctctgctagttccagacacct
cccaagcacgcagcaatgcagctcaaaacgcttagcctagccacacccccacgggaaacagcagtgat
taacctttagcaataaacgaaagtttaactaagctatactaaccccagggttggtcaatttcgtgcca
gccacaccctggagctagcaaaccgctgatcagcctcgactgtgccttctagttgccagccatctgtt
gtttgcccctcccccgtgccttccttgaccctggaaggtgccactcccactgtcctttcctaataaaa
tgaggaaattgcatcgcattgtctgagtaggtgtcattctattctggggggtggggtggggcaggaca
gcaagggggaggattgggaagacaatagcaggcatgctggggatgcggtgggctctatggcttctgag
gcggaaagaaccagctggggctctagggggtatccccacgcgccctgtagcggcgcattaagcgcggc
gggtgtggtggttacgcgcagcgtgaccgctacacttgccagcgccctagcgcccgctcctttcgctt
tcttcccttcctttctcgccacgttcgccggctttccccgtcaagctctaaatcgggggctcccttta
gggttccgatttagtgctttacggcacctcgaccccaaaaaacttgattagggtgatggttcacgtag
tgggccatcgccctgatagacggtttttcgccctttgacgttggagtccacgttctttaatagtggac
tcttgttccaaactggaacaacactcaaccctatctcggtctattcttttgatttataagggattttg
ccgatttcggcctattggttaaaaaatgagctgatttaacaaaaatttaacgcgaattaattctgtgg
aatgtgtgtcagttagggtgtggaaagtccccaggctccccagcaggcagaagtatgcaaagcatgca
tctcaattagtcagcaaccaggtgtggaaagtccccaggctccccagcaggcagaagtatgcaaagca
tgcatctcaattagtcagcaaccatagtcccgcccctaactccgcccatcccgcccctaactccgccc
agttccgcccattctccgccccatggctgactaattttttttatttatgcagaggccgaggccgcctc
tgcctctgagctattccagaagtagtgaggaggcttttttggaggcctaggcttttgcaaaaagctcc
cgggagcttgtatatccattttcggatctgatcaagagacaggatgaggatcgtttcgcatgattgaa
caagatggattgcacgcaggttctccggccgcttgggtggagaggctattcggctatgactgggcaca
acagacaatcggctgctctgatgccgccgtgttccggctgtcagcgcaggggcgcccggttctttttg
tcaagaccgacctgtccggtgccctgaatgaactgcaggacgaggcagcgcggctatcgtggctggcc
acgacgggcgttccttgcgcagctgtgctcgacgttgtcactgaagcgggaagggactggctgctatt
gggcgaagtgccggggcaggatctcctgtcatctcaccttgctcctgccgagaaagtatccatcatgg
ctgatgcaatgcggcggctgcatacgcttgatccggctacctgcccattcgaccaccaagcgaaacat
cgcatcgagcgagcacgtactcggatggaagccggtcttgtcgatcaggatgatctggacgaagagca
tcaggggctcgcgccagccgaactgttcgccaggctcaaggcgcgcatgcccgacggcgaggatctcg
tcgtgacccatggcgatgcctgcttgccgaatatcatggtggaaaatggccgcttttctggattcatc
gactgtggccggctgggtgtggcggaccgctatcaggacatagcgttggctacccgtgatattgctga
agagcttggcggcgaatgggctgaccgcttcctcgtgctttacggtatcgccgctcccgattcgcagc
gcatcgccttctatcgccttcttgacgagttcttctgagcgggactctggggttcgaaatgaccgacc
aagcgacgcccaacctgccatcacgagatttcgattccaccgccgccttctatgaaaggttgggcttc
ggaatcgttttccgggacgccggctggatgatcctccagcgcggggatctcatgctggagttcttcgc
ccaccccaacttgtttattgcagcttataatggttacaaataaagcaatagcatcacaaatttcacaa
ataaagcatttttttcactgcattctagttgtggtttgtccaaactcatcaatgtatcttatcatgtc
tgtataccgtcgacctctagctagagcttggcgtaatcatggtcatagctgtttcctgtgtgaaattg
ttatccgctcacaattccacacaacatacgagccggaagcataaagtgtaaagcctggggtgcctaat
gagtgagctaactcacattaattgcgttgcgctcactgcccgctttccagtcgggaaacctgtcgtgc
cagctgcattaatgaatcggccaacgcgcggggagaggcggtttgcgtattgggcgctcttccgcttc
ctcgctcactgactcgctgcgctcggtcgttcggctgcggcgagcggtatcagctcactcaaaggcgg
taatacggttatccacagaatcaggggataacgcaggaaagaacatgtgagcaaaaggccagcaaaag
gccaggaaccgtaaaaaggccgcgttgctggcgtttttccataggctccgcccccctgacgagcatca
caaaaatcgacgctcaagtcagaggtggcgaaacccgacaggactataaagataccaggcgtttcccc
ctggaagctccctcgtgcgctctcctgttccgaccctgccgcttaccggatacctgtccgcctttctc
ccttcgggaagcgtggcgctttctcatagctcacgctgtaggtatctcagttcggtgtaggtcgttcg
ctccaagctgggctgtgtgcacgaaccccccgttcagcccgaccgctgcgccttatccggtaactatc
gtcttgagtccaacccggtaagacacgacttatcgccactggcagcagccactggtaacaggattagc
agagcgaggtatgtaggcggtgctacagagttcttgaagtggtggcctaactacggctacactagaag
aacagtatttggtatctgcgctctgctgaagccagttaccttcggaaaaagagttggtagctcttgat
ccggcaaacaaaccaccgctggtagcggtggtttttttgtttgcaagcagcagattacgcgcagaaaa
aaaggatctcaagaagatcctttgatcttttctacggggtctgacgctcagtggaacgaaaactcacg
ttaagggattttggtcatgagattatcaaaaaggatcttcacctagatccttttaaattaaaaatgaa
gttttaaatcaatctaaagtatatatgagtaaacttggtctgacagttaccaatgcttaatcagtgag
gcacctatctcagcgatctgtctatttcgttcatccatagttgcctgactccccgtcgtgtagataac
tacgatacgggagggcttaccatctggccccagtgctgcaatgataccgcgagacccacgctcaccgg
ctccagatttatcagcaataaaccagccagccggaagggccgagcgcagaagtggtcctgcaacttta
tccgcctccatccagtctattaattgttgccgggaagctagagtaagtagttcgccagttaatagttt
gcgcaacgttgttgccattgctacaggcatcgtggtgtcacgctcgtcgtttggtatggcttcattca
gctccggttcccaacgatcaaggcgagttacatgatcccccatgttgtgcaaaaaagcggttagctcc
ttcggtcctccgatcgttgtcagaagtaagttggccgcagtgttatcactcatggttatggcagcact
gcataattctcttactgtcatgccatccgtaagatgcttttctgtgactggtgagtactcaaccaagt
cattctgagaatagtgtatgcggcgaccgagttgctcttgcccggcgtcaatacgggataataccgcg
ccacatagcagaactttaaaagtgctcatcattggaaaacgttcttcggggcgaaaactctcaaggat
cttaccgctgttgagatccagttcgatgtaacccactcgtgcacccaactgatcttcagcatctttta
ctttcaccagcgtttctgggtgagcaaaaacaggaaggcaaaatgccgcaaaaaagggaataagggcg
acacggaaatgttgaatactcatactcttcctttttcaatattattgaagcatttatcagggttattg
tctcatgagcggatacatatttgaatgtatttagaaaaataaacaaataggggttccgcgcacatttc
cccgaaaagtgccacctgacgtc
pZV06_SARS-CoV-2-UTR-spike_delta_HexaPro_SPY_catcher_S638_S2
SEQ ID NO: 88
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccgagaataaactagtattcttctggtccccacagactcagagagaaccc
gccaccatgtttgtgttcctggtgctgctgccactggtgtccagcgtgaccacactgtccggactgtc
tggagagcagggaccatccggcgacatgaccacagaggaggattctgccacacacatcaagttcagca
agagggacgaggacggaagagagctggcaggagcaaccatggagctgagggatagctccggcaagacc
atcagcacatggatctccgacggccacgtgaaggatttctacctgtatcccggcaagtacacctttgt
ggagacagcagcaccagacggatatgaggtggcaacccctatcgagtttacagtgaacgaggacggac
aggtgaccgtggatggagaggcaacagagggcgatgcacacaccggcagcagcagcaacgtgttccag
accagggctggctgtctgattggagcagagcatgtgaacaactcctatgagtgtgacatcccaattgg
agcaggcatctgtgcctcctaccagacccagaccaacagcaggggctctgcatcttctgtggcaagcc
agagcatcattgcctacacaatgagtctgggagcagagaactctgtggcttacagcaacaacagcatt
gccatcccaaccaacttcaccatctctgtgaccacagagattctgcctgtgagtatgaccaagacctc
tgtggactgtacaatgtatatctgtggagacagcacagagtgtagcaacctgctgctccaatatggct
ccttctgtacccaacttaacagggctctgacaggcattgctgtggaacaggacaagaacacccaggag
gtgtttgcccaggtgaagcagatttacaagacacctccaatcaaggactttggaggcttcaacttcag
ccagattctgcctgacccaagcaagccaagcaagaggtcccctattgaggacctgctgttcaacaagg
tgaccctggctgatgctggcttcatcaagcaatatggagactgtctgggagacattgccgccagggac
ctgatttgtgcccagaagttcaatggactgacagtgctgcctccactgctgacagatgagatgattgc
ccaatacacctctgccctgctggctggcaccatcacctctggctggacctttggagcaggaccagccc
tccaaatcccatttccaatgcagatggcttacaggttcaatggcattggagtgacccagaatgtgctc
tatgagaaccagaaactgattgccaaccagttcaactctgccattggcaagattcaggactccctgtc
cagcacaccatctgccctgggcaaactccaaaacgtggtgaaccagaatgcccaggctctgaacaccc
tggtgaagcaactttccagcaactttggagccatctcctctgtgctgaatgacatcctgagcagactg
gacccaccagaggctgaggtccagattgacagactgattacaggcagactccaatccctccaaaccta
tgtgacccaacaacttatcagggctgctgagattagggcatctgccaacctggctgccaccaagatga
gtgagtgtgtgctgggacaaagcaagagggtggacttctgtggcaagggctaccacctgatgagtttt
ccacagtctgcccctcatggagtggtgttcctgcatgtgacctatgtgcctgcccaggagaagaactt
caccacagcccctgccatctgccatgatggcaaggctcactttccaagggagggagtgtttgtgagca
atggcacccactggtttgtgacccagaggaacttctatgaaccacagattatcaccacagacaacacc
tttgtgtctggcaactgtgatgtggtgattggcattgtgaacaacacagtctatgacccactccaacc
tgaactggactccttcaaggaggaactggacaaatacttcaagaaccacaccagccctgatgtggacc
tgggagacatctctggcatcaatgcctctgtggtgaacatccagaaggagattgacagactgaatgag
gtggctaagaacctgaatgagtccctgattgacctccaagaactgggcaaatatgaacaatacatcaa
gtggccatggtacatctggctgggcttcattgctggactgattgccattgtgatggtgaccataatgc
tgtgttgtatgacctcctgttgttcctgtctgaaaggctgttgttcctgtggctcctgttgtaagttt
gatgaggatgactctgaacctgtgctgaaaggagtgaaactgcactacacctgactcgagctggtact
gcatgcacgcaatgctagctgcccctttcccgtcctgggtaccccgagtctcccccgacctcgggtcc
caggtatgctcccacctccacctgccccactcaccacctctgctagttccagacacctcccaagcacg
cagcaatgcagctcaaaacgcttagcctagccacacccccacgggaaacagcagtgattaacctttag
caataaacgaaagtttaactaagctatactaaccccagggttggtcaatttcgtgccagccacaccct
ggagctagcaaaccgctgatcagcctcgactgtgccttctagttgccagccatctgttgtttgcccct
cccccgtgccttccttgaccctggaaggtgccactcccactgtcctttcctaataaaatgaggaaatt
gcatcgcattgtctgagtaggtgtcattctattctggggggtggggtggggcaggacagcaaggggga
ggattgggaagacaatagcaggcatgctggggatgcggtgggctctatggcttctgaggcggaaagaa
ccagctggggctctagggggtatccccacgcgccctgtagcggcgcattaagcgcggcgggtgtggtg
gttacgcgcagcgtgaccgctacacttgccagcgccctagcgcccgctcctttcgctttcttcccttc
ctttctcgccacgttcgccggctttccccgtcaagctctaaatcgggggctccctttagggttccgat
ttagtgctttacggcacctcgaccccaaaaaacttgattagggtgatggttcacgtagtgggccatcg
ccctgatagacggtttttcgccctttgacgttggagtccacgttctttaatagtggactcttgttcca
aactggaacaacactcaaccctatctcggtctattcttttgatttataagggattttgccgatttcgg
cctattggttaaaaaatgagctgatttaacaaaaatttaacgcgaattaattctgtggaatgtgtgtc
agttagggtgtggaaagtccccaggctccccagcaggcagaagtatgcaaagcatgcatctcaattag
tcagcaaccaggtgtggaaagtccccaggctccccagcaggcagaagtatgcaaagcatgcatctcaa
ttagtcagcaaccatagtcccgcccctaactccgcccatcccgcccctaactccgcccagttccgccc
attctccgccccatggctgactaattttttttatttatgcagaggccgaggccgcctctgcctctgag
ctattccagaagtagtgaggaggcttttttggaggcctaggcttttgcaaaaagctcccgggagcttg
tatatccattttcggatctgatcaagagacaggatgaggatcgtttcgcatgattgaacaagatggat
tgcacgcaggttctccggccgcttgggtggagaggctattcggctatgactgggcacaacagacaatc
ggctgctctgatgccgccgtgttccggctgtcagcgcaggggcgcccggttctttttgtcaagaccga
cctgtccggtgccctgaatgaactgcaggacgaggcagcgcggctatcgtggctggccacgacgggcg
ttccttgcgcagctgtgctcgacgttgtcactgaagcgggaagggactggctgctattgggcgaagtg
ccggggcaggatctcctgtcatctcaccttgctcctgccgagaaagtatccatcatggctgatgcaat
gcggcggctgcatacgcttgatccggctacctgcccattcgaccaccaagcgaaacatcgcatcgagc
gagcacgtactcggatggaagccggtcttgtcgatcaggatgatctggacgaagagcatcaggggctc
gcgccagccgaactgttcgccaggctcaaggcgcgcatgcccgacggcgaggatctcgtcgtgaccca
tggcgatgcctgcttgccgaatatcatggtggaaaatggccgcttttctggattcatcgactgtggcc
ggctgggtgtggcggaccgctatcaggacatagcgttggctacccgtgatattgctgaagagcttggc
ggcgaatgggctgaccgcttcctcgtgctttacggtatcgccgctcccgattcgcagcgcatcgcctt
ctatcgccttcttgacgagttcttctgagcgggactctggggttcgaaatgaccgaccaagcgacgcc
caacctgccatcacgagatttcgattccaccgccgccttctatgaaaggttgggcttcggaatcgttt
tccgggacgccggctggatgatcctccagcgcggggatctcatgctggagttcttcgcccaccccaac
ttgtttattgcagcttataatggttacaaataaagcaatagcatcacaaatttcacaaataaagcatt
tttttcactgcattctagttgtggtttgtccaaactcatcaatgtatcttatcatgtctgtataccgt
cgacctctagctagagcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaagaacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccacctgacgtc
pZV07_SARS-CoV-2-UTR-spike_delta_HexaPro_S1_S683_furin
SEQ ID NO: 89
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccgagaataaactagtattcttctggtccccacagactcagagagaaccc
gccaccatgtttgtgttcctggtgctgctgccactggtgtccagccagtgtgtgaacctgaggaccag
gacccaacttcctcctgcctacaccaactccttcaccaggggagtctactaccctgacaaggtgttca
ggtcctctgtgctgcacagcacccaggacctgttcctgccattcttcagcaatgtgacctggttccat
gccatccatgtgtctggcaccaatggcaccaagaggtttgacaaccctgtgctgccattcaatgatgg
agtctactttgccagcacagagaagagcaacatcatcaggggctggatttttggcaccaccctggaca
gcaagacccagtccctgctgattgtgaacaatgccaccaatgtggtgattaaggtgtgtgagttccag
ttctgtaatgacccattcctggacgtctactaccacaagaacaacaagtcctggatggagtctggcgt
ctactcctctgccaacaactgtacctttgaatatgtgagccaaccattcctgatggacttggagggca
agcagggcaacttcaagaacctgagggagtttgtgttcaagaacattgatggctacttcaagatttac
agcaaacacacaccaatcaacctggtgagggacctgccacagggcttctctgccttggaaccactggt
ggacctgccaattggcatcaacatcaccaggttccagaccctgctggctctgcacaggtcctacctga
cacctggagactcctcctctggctggacagcaggagcagcagcctactatgtgggctacctccaacca
aggaccttcctgctgaaatacaatgagaatggcaccatcacagatgctgtggactgtgccctggaccc
actgtctgagaccaagtgtaccctgaaatccttcacagtggagaagggcatctaccagaccagcaact
tcagggtccaaccaacagagagcattgtgaggtttccaaacatcaccaacctgtgtccatttggagag
gtgttcaatgccaccaggtttgcctctgtctatgcctggaacaggaagaggattagcaactgtgtggc
tgactactctgtgctctacaactctgcctccttcagcaccttcaagtgttatggagtgagcccaacca
aactgaatgacctgtgtttcaccaatgtctatgctgactcctttgtgattaggggagatgaggtgaga
cagattgcccctggacaaacaggcaagattgctgactacaactacaaactgcctgatgacttcacagg
ctgtgtgattgcctggaacagcaacaacctggacagcaaggtgggaggcaactacaactacaggtaca
gactgttcaggaagagcaacctgaaaccatttgagagggacatcagcacagagatttaccaggctggc
agcaagccatgtaatggagtggagggcttcaactgttactttccactccaatcctatggcttccaacc
aaccaatggagtgggctaccaaccatacagggtggtggtgctgtcctttgaactgctccatgcccctg
ccacagtgtgtggaccaaagaagagcaccaacctggtgaagaacaagtgtgtgaacttcaacttcaat
ggactgacaggcacaggagtgctgacagagagcaacaagaagttcctgccattccaacagtttggcag
ggacattgctgacaccacagatgctgtgagggacccacagaccttggagattctggacatcacaccat
gttcctttggaggagtgtctgtgattacacctggcaccaacaccagcaaccaggtggctgtgctctac
cagggcgtgaactgtactgaggtgcctgtggctatccatgctgaccaacttacaccaacctggagggt
ctacagcacaggaagcaacgtgttccagaccagggctggctgtctgattggagcagagcatgtgaaca
actcctatgagtgtgacatcccaattggagcaggcatctgtgcctcctaccagacccagaccaacagc
aggggctctgcatcttgactcgagctggtactgcatgcacgcaatgctagctgcccctttcccgtcct
gggtaccccgagtctcccccgacctcgggtcccaggtatgctcccacctccacctgccccactcacca
cctctgctagttccagacacctcccaagcacgcagcaatgcagctcaaaacgcttagcctagccacac
ccccacgggaaacagcagtgattaacctttagcaataaacgaaagtttaactaagctatactaacccc
agggttggtcaatttcgtgccagccacaccctggagctagcaaaccgctgatcagcctcgactgtgcc
ttctagttgccagccatctgttgtttgcccctcccccgtgccttccttgaccctggaaggtgccactc
ccactgtcctttcctaataaaatgaggaaattgcatcgcattgtctgagtaggtgtcattctattctg
gggggtggggtggggcaggacagcaagggggaggattgggaagacaatagcaggcatgctggggatgc
ggtgggctctatggcttctgaggcggaaagaaccagctggggctctagggggtatccccacgcgccct
gtagcggcgcattaagcgcggcgggtgtggtggttacgcgcagcgtgaccgctacacttgccagcgcc
ctagcgcccgctcctttcgctttcttcccttcctttctcgccacgttcgccggctttccccgtcaagc
tctaaatcgggggctccctttagggttccgatttagtgctttacggcacctcgaccccaaaaaacttg
attagggtgatggttcacgtagtgggccatcgccctgatagacggtttttcgccctttgacgttggag
tccacgttctttaatagtggactcttgttccaaactggaacaacactcaaccctatctcggtctattc
ttttgatttataagggattttgccgatttcggcctattggttaaaaaatgagctgatttaacaaaaat
ttaacgcgaattaattctgtggaatgtgtgtcagttagggtgtggaaagtccccaggctccccagcag
gcagaagtatgcaaagcatgcatctcaattagtcagcaaccaggtgtggaaagtccccaggctcccca
gcaggcagaagtatgcaaagcatgcatctcaattagtcagcaaccatagtcccgcccctaactccgcc
catcccgcccctaactccgcccagttccgcccattctccgccccatggctgactaattttttttattt
atgcagaggccgaggccgcctctgcctctgagctattccagaagtagtgaggaggcttttttggaggc
ctaggcttttgcaaaaagctcccgggagcttgtatatccattttcggatctgatcaagagacaggatg
aggatcgtttcgcatgattgaacaagatggattgcacgcaggttctccggccgcttgggtggagaggc
tattcggctatgactgggcacaacagacaatcggctgctctgatgccgccgtgttccggctgtcagcg
caggggcgcccggttctttttgtcaagaccgacctgtccggtgccctgaatgaactgcaggacgaggc
agcgcggctatcgtggctggccacgacgggcgttccttgcgcagctgtgctcgacgttgtcactgaag
cgggaagggactggctgctattgggcgaagtgccggggcaggatctcctgtcatctcaccttgctcct
gccgagaaagtatccatcatggctgatgcaatgcggcggctgcatacgcttgatccggctacctgccc
attcgaccaccaagcgaaacatcgcatcgagcgagcacgtactcggatggaagccggtcttgtcgatc
aggatgatctggacgaagagcatcaggggctcgcgccagccgaactgttcgccaggctcaaggcgcgc
atgcccgacggcgaggatctcgtcgtgacccatggcgatgcctgcttgccgaatatcatggtggaaaa
tggccgcttttctggattcatcgactgtggccggctgggtgtggcggaccgctatcaggacatagcgt
tggctacccgtgatattgctgaagagcttggcggcgaatgggctgaccgcttcctcgtgctttacggt
atcgccgctcccgattcgcagcgcatcgccttctatcgccttcttgacgagttcttctgagcgggact
ctggggttcgaaatgaccgaccaagcgacgcccaacctgccatcacgagatttcgattccaccgccgc
cttctatgaaaggttgggcttcggaatcgttttccgggacgccggctggatgatcctccagcgcgggg
atctcatgctggagttcttcgcccaccccaacttgtttattgcagcttataatggttacaaataaagc
aatagcatcacaaatttcacaaataaagcatttttttcactgcattctagttgtggtttgtccaaact
catcaatgtatcttatcatgtctgtataccgtcgacctctagctagagcttggcgtaatcatggtcat
agctgtttcctgtgtgaaattgttatccgctcacaattccacacaacatacgagccggaagcataaag
tgtaaagcctggggtgcctaatgagtgagctaactcacattaattgcgttgcgctcactgcccgcttt
ccagtcgggaaacctgtcgtgccagctgcattaatgaatcggccaacgcgcggggagaggcggtttgc
gtattgggcgctcttccgcttcctcgctcactgactcgctgcgctcggtcgttcggctgcggcgagcg
gtatcagctcactcaaaggcggtaatacggttatccacagaatcaggggataacgcaggaaagaacat
gtgagcaaaaggccagcaaaaggccaggaaccgtaaaaaggccgcgttgctggcgtttttccataggc
tccgcccccctgacgagcatcacaaaaatcgacgctcaagtcagaggtggcgaaacccgacaggacta
taaagataccaggcgtttccccctggaagctccctcgtgcgctctcctgttccgaccctgccgcttac
cggatacctgtccgcctttctcccttcgggaagcgtggcgctttctcatagctcacgctgtaggtatc
tcagttcggtgtaggtcgttcgctccaagctgggctgtgtgcacgaaccccccgttcagcccgaccgc
tgcgccttatccggtaactatcgtcttgagtccaacccggtaagacacgacttatcgccactggcagc
agccactggtaacaggattagcagagcgaggtatgtaggcggtgctacagagttcttgaagtggtggc
ctaactacggctacactagaagaacagtatttggtatctgcgctctgctgaagccagttaccttcgga
aaaagagttggtagctcttgatccggcaaacaaaccaccgctggtagcggtggtttttttgtttgcaa
gcagcagattacgcgcagaaaaaaaggatctcaagaagatcctttgatcttttctacggggtctgacg
ctcagtggaacgaaaactcacgttaagggattttggtcatgagattatcaaaaaggatcttcacctag
atccttttaaattaaaaatgaagttttaaatcaatctaaagtatatatgagtaaacttggtctgacag
ttaccaatgcttaatcagtgaggcacctatctcagcgatctgtctatttcgttcatccatagttgcct
gactccccgtcgtgtagataactacgatacgggagggcttaccatctggccccagtgctgcaatgata
ccgcgagacccacgctcaccggctccagatttatcagcaataaaccagccagccggaagggccgagcg
cagaagtggtcctgcaactttatccgcctccatccagtctattaattgttgccgggaagctagagtaa
gtagttcgccagttaatagtttgcgcaacgttgttgccattgctacaggcatcgtggtgtcacgctcg
tcgtttggtatggcttcattcagctccggttcccaacgatcaaggcgagttacatgatcccccatgtt
gtgcaaaaaagcggttagctccttcggtcctccgatcgttgtcagaagtaagttggccgcagtgttat
cactcatggttatggcagcactgcataattctcttactgtcatgccatccgtaagatgcttttctgtg
actggtgagtactcaaccaagtcattctgagaatagtgtatgcggcgaccgagttgctcttgcccggc
gtcaatacgggataataccgcgccacatagcagaactttaaaagtgctcatcattggaaaacgttctt
cggggcgaaaactctcaaggatcttaccgctgttgagatccagttcgatgtaacccactcgtgcaccc
aactgatcttcagcatcttttactttcaccagcgtttctgggtgagcaaaaacaggaaggcaaaatgc
cgcaaaaaagggaataagggcgacacggaaatgttgaatactcatactcttcctttttcaatattatt
gaagcatttatcagggttattgtctcatgagcggatacatatttgaatgtatttagaaaaataaacaa
ataggggttccgcgcacatttccccgaaaagtgccacctgacgtc
pZV08_SARS-CoV-2-UTR-spike_delta_HexaPro_S684_S2
SEQ ID NO: 90
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccgagaataaactagtattcttctggtccccacagactcagagagaaccc
gccaccatgtttgtgttcctggtgctgctgccactggtgtccagctctgtggcaagccagagcatcat
tgcctacacaatgagtctgggagcagagaactctgtggcttacagcaacaacagcattgccatcccaa
ccaacttcaccatctctgtgaccacagagattctgcctgtgagtatgaccaagacctctgtggactgt
acaatgtatatctgtggagacagcacagagtgtagcaacctgctgctccaatatggctccttctgtac
ccaacttaacagggctctgacaggcattgctgtggaacaggacaagaacacccaggaggtgtttgccc
aggtgaagcagatttacaagacacctccaatcaaggactttggaggcttcaacttcagccagattctg
cctgacccaagcaagccaagcaagaggtcccctattgaggacctgctgttcaacaaggtgaccctggc
tgatgctggcttcatcaagcaatatggagactgtctgggagacattgccgccagggacctgatttgtg
cccagaagttcaatggactgacagtgctgcctccactgctgacagatgagatgattgcccaatacacc
tctgccctgctggctggcaccatcacctctggctggacctttggagcaggaccagccctccaaatccc
atttccaatgcagatggcttacaggttcaatggcattggagtgacccagaatgtgctctatgagaacc
agaaactgattgccaaccagttcaactctgccattggcaagattcaggactccctgtccagcacacca
tctgccctgggcaaactccaaaacgtggtgaaccagaatgcccaggctctgaacaccctggtgaagca
actttccagcaactttggagccatctcctctgtgctgaatgacatcctgagcagactggacccaccag
aggctgaggtccagattgacagactgattacaggcagactccaatccctccaaacctatgtgacccaa
caacttatcagggctgctgagattagggcatctgccaacctggctgccaccaagatgagtgagtgtgt
gctgggacaaagcaagagggtggacttctgtggcaagggctaccacctgatgagttttccacagtctg
cccctcatggagtggtgttcctgcatgtgacctatgtgcctgcccaggagaagaacttcaccacagcc
cctgccatctgccatgatggcaaggctcactttccaagggagggagtgtttgtgagcaatggcaccca
ctggtttgtgacccagaggaacttctatgaaccacagattatcaccacagacaacacctttgtgtctg
gcaactgtgatgtggtgattggcattgtgaacaacacagtctatgacccactccaacctgaactggac
tccttcaaggaggaactggacaaatacttcaagaaccacaccagccctgatgtggacctgggagacat
ctctggcatcaatgcctctgtggtgaacatccagaaggagattgacagactgaatgaggtggctaaga
acctgaatgagtccctgattgacctccaagaactgggcaaatatgaacaatacatcaagtggccatgg
tacatctggctgggcttcattgctggactgattgccattgtgatggtgaccataatgctgtgttgtat
gacctcctgttgttcctgtctgaaaggctgttgttcctgtggctcctgttgtaagtttgatgaggatg
actctgaacctgtgctgaaaggagtgaaactgcactacacctgactcgagctggtactgcatgcacgc
aatgctagctgcccctttcccgtcctgggtaccccgagtctcccccgacctcgggtcccaggtatgct
cccacctccacctgccccactcaccacctctgctagttccagacacctcccaagcacgcagcaatgca
gctcaaaacgcttagcctagccacacccccacgggaaacagcagtgattaacctttagcaataaacga
aagtttaactaagctatactaaccccagggttggtcaatttcgtgccagccacaccctggagctagca
aaccgctgatcagcctcgactgtgccttctagttgccagccatctgttgtttgcccctcccccgtgcc
ttccttgaccctggaaggtgccactcccactgtcctttcctaataaaatgaggaaattgcatcgcatt
gtctgagtaggtgtcattctattctggggggtggggtggggcaggacagcaagggggaggattgggaa
gacaatagcaggcatgctggggatgcggtgggctctatggcttctgaggcggaaagaaccagctgggg
ctctagggggtatccccacgcgccctgtagcggcgcattaagcgcggcgggtgtggtggttacgcgca
gcgtgaccgctacacttgccagcgccctagcgcccgctcctttcgctttcttcccttcctttctcgcc
acgttcgccggctttccccgtcaagctctaaatcgggggctccctttagggttccgatttagtgcttt
acggcacctcgaccccaaaaaacttgattagggtgatggttcacgtagtgggccatcgccctgataga
cggtttttcgccctttgacgttggagtccacgttctttaatagtggactcttgttccaaactggaaca
acactcaaccctatctcggtctattcttttgatttataagggattttgccgatttcggcctattggtt
aaaaaatgagctgatttaacaaaaatttaacgcgaattaattctgtggaatgtgtgtcagttagggtg
tggaaagtccccaggctccccagcaggcagaagtatgcaaagcatgcatctcaattagtcagcaacca
ggtgtggaaagtccccaggctccccagcaggcagaagtatgcaaagcatgcatctcaattagtcagca
accatagtcccgcccctaactccgcccatcccgcccctaactccgcccagttccgcccattctccgcc
ccatggctgactaattttttttatttatgcagaggccgaggccgcctctgcctctgagctattccaga
agtagtgaggaggcttttttggaggcctaggcttttgcaaaaagctcccgggagcttgtatatccatt
ttcggatctgatcaagagacaggatgaggatcgtttcgcatgattgaacaagatggattgcacgcagg
ttctccggccgcttgggtggagaggctattcggctatgactgggcacaacagacaatcggctgctctg
atgccgccgtgttccggctgtcagcgcaggggcgcccggttctttttgtcaagaccgacctgtccggt
gccctgaatgaactgcaggacgaggcagcgcggctatcgtggctggccacgacgggcgttccttgcgc
agctgtgctcgacgttgtcactgaagcgggaagggactggctgctattgggcgaagtgccggggcagg
atctcctgtcatctcaccttgctcctgccgagaaagtatccatcatggctgatgcaatgcggcggctg
catacgcttgatccggctacctgcccattcgaccaccaagcgaaacatcgcatcgagcgagcacgtac
tcggatggaagccggtcttgtcgatcaggatgatctggacgaagagcatcaggggctcgcgccagccg
aactgttcgccaggctcaaggcgcgcatgcccgacggcgaggatctcgtcgtgacccatggcgatgcc
tgcttgccgaatatcatggtggaaaatggccgcttttctggattcatcgactgtggccggctgggtgt
ggcggaccgctatcaggacatagcgttggctacccgtgatattgctgaagagcttggcggcgaatggg
ctgaccgcttcctcgtgctttacggtatcgccgctcccgattcgcagcgcatcgccttctatcgcctt
cttgacgagttcttctgagcgggactctggggttcgaaatgaccgaccaagcgacgcccaacctgcca
tcacgagatttcgattccaccgccgccttctatgaaaggttgggcttcggaatcgttttccgggacgc
cggctggatgatcctccagcgcggggatctcatgctggagttcttcgcccaccccaacttgtttattg
cagcttataatggttacaaataaagcaatagcatcacaaatttcacaaataaagcatttttttcactg
cattctagttgtggtttgtccaaactcatcaatgtatcttatcatgtctgtataccgtcgacctctag
ctagagcttggcgtaatcatggtcatagctgtttcctgtgtgaaattgttatccgctcacaattccac
acaacatacgagccggaagcataaagtgtaaagcctggggtgcctaatgagtgagctaactcacatta
attgcgttgcgctcactgcccgctttccagtcgggaaacctgtcgtgccagctgcattaatgaatcgg
ccaacgcgcggggagaggcggtttgcgtattgggcgctcttccgcttcctcgctcactgactcgctgc
gctcggtcgttcggctgcggcgagcggtatcagctcactcaaaggcggtaatacggttatccacagaa
tcaggggataacgcaggaaagaacatgtgagcaaaaggccagcaaaaggccaggaaccgtaaaaaggc
cgcgttgctggcgtttttccataggctccgcccccctgacgagcatcacaaaaatcgacgctcaagtc
agaggtggcgaaacccgacaggactataaagataccaggcgtttccccctggaagctccctcgtgcgc
tctcctgttccgaccctgccgcttaccggatacctgtccgcctttctcccttcgggaagcgtggcgct
ttctcatagctcacgctgtaggtatctcagttcggtgtaggtcgttcgctccaagctgggctgtgtgc
acgaaccccccgttcagcccgaccgctgcgccttatccggtaactatcgtcttgagtccaacccggta
agacacgacttatcgccactggcagcagccactggtaacaggattagcagagcgaggtatgtaggcgg
tgctacagagttcttgaagtggtggcctaactacggctacactagaagaacagtatttggtatctgcg
ctctgctgaagccagttaccttcggaaaaagagttggtagctcttgatccggcaaacaaaccaccgct
ggtagcggtggtttttttgtttgcaagcagcagattacgcgcagaaaaaaaggatctcaagaagatcc
tttgatcttttctacggggtctgacgctcagtggaacgaaaactcacgttaagggattttggtcatga
gattatcaaaaaggatcttcacctagatccttttaaattaaaaatgaagttttaaatcaatctaaagt
atatatgagtaaacttggtctgacagttaccaatgcttaatcagtgaggcacctatctcagcgatctg
tctatttcgttcatccatagttgcctgactccccgtcgtgtagataactacgatacgggagggcttac
catctggccccagtgctgcaatgataccgcgagacccacgctcaccggctccagatttatcagcaata
aaccagccagccggaagggccgagcgcagaagtggtcctgcaactttatccgcctccatccagtctat
taattgttgccgggaagctagagtaagtagttcgccagttaatagtttgcgcaacgttgttgccattg
ctacaggcatcgtggtgtcacgctcgtcgtttggtatggcttcattcagctccggttcccaacgatca
aggcgagttacatgatcccccatgttgtgcaaaaaagcggttagctccttcggtcctccgatcgttgt
cagaagtaagttggccgcagtgttatcactcatggttatggcagcactgcataattctcttactgtca
tgccatccgtaagatgcttttctgtgactggtgagtactcaaccaagtcattctgagaatagtgtatg
cggcgaccgagttgctcttgcccggcgtcaatacgggataataccgcgccacatagcagaactttaaa
agtgctcatcattggaaaacgttcttcggggcgaaaactctcaaggatcttaccgctgttgagatcca
gttcgatgtaacccactcgtgcacccaactgatcttcagcatcttttactttcaccagcgtttctggg
tgagcaaaaacaggaaggcaaaatgccgcaaaaaagggaataagggcgacacggaaatgttgaatact
catactcttcctttttcaatattattgaagcatttatcagggttattgtctcatgagcggatacatat
ttgaatgtatttagaaaaataaacaaataggggttccgcgcacatttccccgaaaagtgccacctgac
gtc
pZV09_SARS-CoV-2-UTR-spike_delta_HexaPro_S684_S2_del_C19
SEQ ID NO: 91
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccgagaataaactagtattcttctggtccccacagactcagagagaaccc
gccaccatgtttgtgttcctggtgctgctgccactggtgtccagctctgtggcaagccagagcatcat
tgcctacacaatgagtctgggagcagagaactctgtggcttacagcaacaacagcattgccatcccaa
ccaacttcaccatctctgtgaccacagagattctgcctgtgagtatgaccaagacctctgtggactgt
acaatgtatatctgtggagacagcacagagtgtagcaacctgctgctccaatatggctccttctgtac
ccaacttaacagggctctgacaggcattgctgtggaacaggacaagaacacccaggaggtgtttgccc
aggtgaagcagatttacaagacacctccaatcaaggactttggaggcttcaacttcagccagattctg
cctgacccaagcaagccaagcaagaggtcccctattgaggacctgctgttcaacaaggtgaccctggc
tgatgctggcttcatcaagcaatatggagactgtctgggagacattgccgccagggacctgatttgtg
cccagaagttcaatggactgacagtgctgcctccactgctgacagatgagatgattgcccaatacacc
tctgccctgctggctggcaccatcacctctggctggacctttggagcaggaccagccctccaaatccc
atttccaatgcagatggcttacaggttcaatggcattggagtgacccagaatgtgctctatgagaacc
agaaactgattgccaaccagttcaactctgccattggcaagattcaggactccctgtccagcacacca
tctgccctgggcaaactccaaaacgtggtgaaccagaatgcccaggctctgaacaccctggtgaagca
actttccagcaactttggagccatctcctctgtgctgaatgacatcctgagcagactggacccaccag
aggctgaggtccagattgacagactgattacaggcagactccaatccctccaaacctatgtgacccaa
caacttatcagggctgctgagattagggcatctgccaacctggctgccaccaagatgagtgagtgtgt
gctgggacaaagcaagagggtggacttctgtggcaagggctaccacctgatgagttttccacagtctg
cccctcatggagtggtgttcctgcatgtgacctatgtgcctgcccaggagaagaacttcaccacagcc
cctgccatctgccatgatggcaaggctcactttccaagggagggagtgtttgtgagcaatggcaccca
ctggtttgtgacccagaggaacttctatgaaccacagattatcaccacagacaacacctttgtgtctg
gcaactgtgatgtggtgattggcattgtgaacaacacagtctatgacccactccaacctgaactggac
tccttcaaggaggaactggacaaatacttcaagaaccacaccagccctgatgtggacctgggagacat
ctctggcatcaatgcctctgtggtgaacatccagaaggagattgacagactgaatgaggtggctaaga
acctgaatgagtccctgattgacctccaagaactgggcaaatatgaacaatacatcaagtggccatgg
tacatctggctgggcttcattgctggactgattgccattgtgatggtgaccataatgctgtgttgtat
gacctcctgttgttcctgtctgaaaggctgttgttcctgtggctcctgttgttgactcgagctggtac
tgcatgcacgcaatgctagctgcccctttcccgtcctgggtaccccgagtctcccccgacctcgggtc
ccaggtatgctcccacctccacctgccccactcaccacctctgctagttccagacacctcccaagcac
gcagcaatgcagctcaaaacgcttagcctagccacacccccacgggaaacagcagtgattaaccttta
gcaataaacgaaagtttaactaagctatactaaccccagggttggtcaatttcgtgccagccacaccc
tggagctagcaaaccgctgatcagcctcgactgtgccttctagttgccagccatctgttgtttgcccc
tcccccgtgccttccttgaccctggaaggtgccactcccactgtcctttcctaataaaatgaggaaat
tgcatcgcattgtctgagtaggtgtcattctattctggggggtggggtggggcaggacagcaaggggg
aggattgggaagacaatagcaggcatgctggggatgcggtgggctctatggcttctgaggcggaaaga
accagctggggctctagggggtatccccacgcgccctgtagcggcgcattaagcgcggcgggtgtggt
ggttacgcgcagcgtgaccgctacacttgccagcgccctagcgcccgctcctttcgctttcttccctt
cctttctcgccacgttcgccggctttccccgtcaagctctaaatcgggggctccctttagggttccga
tttagtgctttacggcacctcgaccccaaaaaacttgattagggtgatggttcacgtagtgggccatc
gccctgatagacggtttttcgccctttgacgttggagtccacgttctttaatagtggactcttgttcc
aaactggaacaacactcaaccctatctcggtctattcttttgatttataagggattttgccgatttcg
gcctattggttaaaaaatgagctgatttaacaaaaatttaacgcgaattaattctgtggaatgtgtgt
cagttagggtgtggaaagtccccaggctccccagcaggcagaagtatgcaaagcatgcatctcaatta
gtcagcaaccaggtgtggaaagtccccaggctccccagcaggcagaagtatgcaaagcatgcatctca
attagtcagcaaccatagtcccgcccctaactccgcccatcccgcccctaactccgcccagttccgcc
cattctccgccccatggctgactaattttttttatttatgcagaggccgaggccgcctctgcctctga
gctattccagaagtagtgaggaggcttttttggaggcctaggcttttgcaaaaagctcccgggagctt
gtatatccattttcggatctgatcaagagacaggatgaggatcgtttcgcatgattgaacaagatgga
ttgcacgcaggttctccggccgcttgggtggagaggctattcggctatgactgggcacaacagacaat
cggctgctctgatgccgccgtgttccggctgtcagcgcaggggcgcccggttctttttgtcaagaccg
acctgtccggtgccctgaatgaactgcaggacgaggcagcgcggctatcgtggctggccacgacgggc
gttccttgcgcagctgtgctcgacgttgtcactgaagcgggaagggactggctgctattgggcgaagt
gccggggcaggatctcctgtcatctcaccttgctcctgccgagaaagtatccatcatggctgatgcaa
tgcggcggctgcatacgcttgatccggctacctgcccattcgaccaccaagcgaaacatcgcatcgag
cgagcacgtactcggatggaagccggtcttgtcgatcaggatgatctggacgaagagcatcaggggct
cgcgccagccgaactgttcgccaggctcaaggcgcgcatgcccgacggcgaggatctcgtcgtgaccc
atggcgatgcctgcttgccgaatatcatggtggaaaatggccgcttttctggattcatcgactgtggc
cggctgggtgtggcggaccgctatcaggacatagcgttggctacccgtgatattgctgaagagcttgg
cggcgaatgggctgaccgcttcctcgtgctttacggtatcgccgctcccgattcgcagcgcatcgcct
tctatcgccttcttgacgagttcttctgagcgggactctggggttcgaaatgaccgaccaagcgacgc
ccaacctgccatcacgagatttcgattccaccgccgccttctatgaaaggttgggcttcggaatcgtt
ttccgggacgccggctggatgatcctccagcgcggggatctcatgctggagttcttcgcccaccccaa
cttgtttattgcagcttataatggttacaaataaagcaatagcatcacaaatttcacaaataaagcat
ttttttcactgcattctagttgtggtttgtccaaactcatcaatgtatcttatcatgtctgtataccg
tcgacctctagctagagcttggcgtaatcatggtcatagctgtttcctgtgtgaaattgttatccgct
cacaattccacacaacatacgagccggaagcataaagtgtaaagcctggggtgcctaatgagtgagct
aactcacattaattgcgttgcgctcactgcccgctttccagtcgggaaacctgtcgtgccagctgcat
taatgaatcggccaacgcgcggggagaggcggtttgcgtattgggcgctcttccgcttcctcgctcac
tgactcgctgcgctcggtcgttcggctgcggcgagcggtatcagctcactcaaaggcggtaatacggt
tatccacagaatcaggggataacgcaggaaagaacatgtgagcaaaaggccagcaaaaggccaggaac
cgtaaaaaggccgcgttgctggcgtttttccataggctccgcccccctgacgagcatcacaaaaatcg
acgctcaagtcagaggtggcgaaacccgacaggactataaagataccaggcgtttccccctggaagct
ccctcgtgcgctctcctgttccgaccctgccgcttaccggatacctgtccgcctttctcccttcggga
agcgtggcgctttctcatagctcacgctgtaggtatctcagttcggtgtaggtcgttcgctccaagct
gggctgtgtgcacgaaccccccgttcagcccgaccgctgcgccttatccggtaactatcgtcttgagt
ccaacccggtaagacacgacttatcgccactggcagcagccactggtaacaggattagcagagcgagg
tatgtaggcggtgctacagagttcttgaagtggtggcctaactacggctacactagaagaacagtatt
tggtatctgcgctctgctgaagccagttaccttcggaaaaagagttggtagctcttgatccggcaaac
aaaccaccgctggtagcggtggtttttttgtttgcaagcagcagattacgcgcagaaaaaaaggatct
caagaagatcctttgatcttttctacggggtctgacgctcagtggaacgaaaactcacgttaagggat
tttggtcatgagattatcaaaaaggatcttcacctagatccttttaaattaaaaatgaagttttaaat
caatctaaagtatatatgagtaaacttggtctgacagttaccaatgcttaatcagtgaggcacctatc
tcagcgatctgtctatttcgttcatccatagttgcctgactccccgtcgtgtagataactacgatacg
ggagggcttaccatctggccccagtgctgcaatgataccgcgagacccacgctcaccggctccagatt
tatcagcaataaaccagccagccggaagggccgagcgcagaagtggtcctgcaactttatccgcctcc
atccagtctattaattgttgccgggaagctagagtaagtagttcgccagttaatagtttgcgcaacgt
tgttgccattgctacaggcatcgtggtgtcacgctcgtcgtttggtatggcttcattcagctccggtt
cccaacgatcaaggcgagttacatgatcccccatgttgtgcaaaaaagcggttagctccttcggtcct
ccgatcgttgtcagaagtaagttggccgcagtgttatcactcatggttatggcagcactgcataattc
tcttactgtcatgccatccgtaagatgcttttctgtgactggtgagtactcaaccaagtcattctgag
aatagtgtatgcggcgaccgagttgctcttgcccggcgtcaatacgggataataccgcgccacatagc
agaactttaaaagtgctcatcattggaaaacgttcttcggggcgaaaactctcaaggatcttaccgct
gttgagatccagttcgatgtaacccactcgtgcacccaactgatcttcagcatcttttactttcacca
gcgtttctgggtgagcaaaaacaggaaggcaaaatgccgcaaaaaagggaataagggcgacacggaaa
tgttgaatactcatactcttcctttttcaatattattgaagcatttatcagggttattgtctcatgag
cggatacatatttgaatgtatttagaaaaataaacaaataggggttccgcgcacatttccccgaaaag
tgccacctgacgtc
pZV10_SARS-CoV-2-UTR-spike_delta_HexaPro_NTD_T313_SPY_catcher
SEQ ID NO: 92
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccgagaataaactagtattcttctggtccccacagactcagagagaaccc
gccaccatgtttgtgttcctggtgctgctgccactggtgtccagccagtgtgtgaacctgaggaccag
gacccaacttcctcctgcctacaccaactccttcaccaggggagtctactaccctgacaaggtgttca
ggtcctctgtgctgcacagcacccaggacctgttcctgccattcttcagcaatgtgacctggttccat
gccatccatgtgtctggcaccaatggcaccaagaggtttgacaaccctgtgctgccattcaatgatgg
agtctactttgccagcacagagaagagcaacatcatcaggggctggatttttggcaccaccctggaca
gcaagacccagtccctgctgattgtgaacaatgccaccaatgtggtgattaaggtgtgtgagttccag
ttctgtaatgacccattcctggacgtctactaccacaagaacaacaagtcctggatggagtctggcgt
ctactcctctgccaacaactgtacctttgaatatgtgagccaaccattcctgatggacttggagggca
agcagggcaacttcaagaacctgagggagtttgtgttcaagaacattgatggctacttcaagatttac
agcaaacacacaccaatcaacctggtgagggacctgccacagggcttctctgccttggaaccactggt
ggacctgccaattggcatcaacatcaccaggttccagaccctgctggctctgcacaggtcctacctga
cacctggagactcctcctctggctggacagcaggagcagcagcctactatgtgggctacctccaacca
aggaccttcctgctgaaatacaatgagaatggcaccatcacagatgctgtggactgtgccctggaccc
actgtctgagaccaagtgtaccctgaagtccttcacagtggagaagggcatctaccagaccggcagca
gcgtgaccacactgtccggactgtctggagagcagggaccatccggcgacatgaccacagaggaggat
tctgccacacacatcaagttcagcaagagggacgaggacggaagagagctggcaggagcaaccatgga
gctgagggatagctccggcaagaccatcagcacatggatctccgacggccacgtgaaggatttctacc
tgtatcccggcaagtacacctttgtggagacagccgcaccagacggatatgaggtggcaacccctatc
gagtttacagtgaacgaggacggacaggtgaccgtggatggagaggcaacagagggcgatgcacacac
ctgactcgagctggtactgcatgcacgcaatgctagctgcccctttcccgtcctgggtaccccgagtc
tcccccgacctcgggtcccaggtatgctcccacctccacctgccccactcaccacctctgctagttcc
agacacctcccaagcacgcagcaatgcagctcaaaacgcttagcctagccacacccccacgggaaaca
gcagtgattaacctttagcaataaacgaaagtttaactaagctatactaaccccagggttggtcaatt
tcgtgccagccacaccctggagctagcaaaccgctgatcagcctcgactgtgccttctagttgccagc
catctgttgtttgcccctcccccgtgccttccttgaccctggaaggtgccactcccactgtcctttcc
taataaaatgaggaaattgcatcgcattgtctgagtaggtgtcattctattctggggggtggggtggg
gcaggacagcaagggggaggattgggaagacaatagcaggcatgctggggatgcggtgggctctatgg
cttctgaggcggaaagaaccagctggggctctagggggtatccccacgcgccctgtagcggcgcatta
agcgcggcgggtgtggtggttacgcgcagcgtgaccgctacacttgccagcgccctagcgcccgctcc
tttcgctttcttcccttcctttctcgccacgttcgccggctttccccgtcaagctctaaatcgggggc
tccctttagggttccgatttagtgctttacggcacctcgaccccaaaaaacttgattagggtgatggt
tcacgtagtgggccatcgccctgatagacggtttttcgccctttgacgttggagtccacgttctttaa
tagtggactcttgttccaaactggaacaacactcaaccctatctcggtctattcttttgatttataag
ggattttgccgatttcggcctattggttaaaaaatgagctgatttaacaaaaatttaacgcgaattaa
ttctgtggaatgtgtgtcagttagggtgtggaaagtccccaggctccccagcaggcagaagtatgcaa
agcatgcatctcaattagtcagcaaccaggtgtggaaagtccccaggctccccagcaggcagaagtat
gcaaagcatgcatctcaattagtcagcaaccatagtcccgcccctaactccgcccatcccgcccctaa
ctccgcccagttccgcccattctccgccccatggctgactaattttttttatttatgcagaggccgag
gccgcctctgcctctgagctattccagaagtagtgaggaggcttttttggaggcctaggcttttgcaa
aaagctcccgggagcttgtatatccattttcggatctgatcaagagacaggatgaggatcgtttcgca
tgattgaacaagatggattgcacgcaggttctccggccgcttgggtggagaggctattcggctatgac
tgggcacaacagacaatcggctgctctgatgccgccgtgttccggctgtcagcgcaggggcgcccggt
tctttttgtcaagaccgacctgtccggtgccctgaatgaactgcaggacgaggcagcgcggctatcgt
ggctggccacgacgggcgttccttgcgcagctgtgctcgacgttgtcactgaagcgggaagggactgg
ctgctattgggcgaagtgccggggcaggatctcctgtcatctcaccttgctcctgccgagaaagtatc
catcatggctgatgcaatgcggcggctgcatacgcttgatccggctacctgcccattcgaccaccaag
cgaaacatcgcatcgagcgagcacgtactcggatggaagccggtcttgtcgatcaggatgatctggac
gaagagcatcaggggctcgcgccagccgaactgttcgccaggctcaaggcgcgcatgcccgacggcga
ggatctcgtcgtgacccatggcgatgcctgcttgccgaatatcatggtggaaaatggccgcttttctg
gattcatcgactgtggccggctgggtgtggcggaccgctatcaggacatagcgttggctacccgtgat
attgctgaagagcttggcggcgaatgggctgaccgcttcctcgtgctttacggtatcgccgctcccga
ttcgcagcgcatcgccttctatcgccttcttgacgagttcttctgagcgggactctggggttcgaaat
gaccgaccaagcgacgcccaacctgccatcacgagatttcgattccaccgccgccttctatgaaaggt
tgggcttcggaatcgttttccgggacgccggctggatgatcctccagcgcggggatctcatgctggag
ttcttcgcccaccccaacttgtttattgcagcttataatggttacaaataaagcaatagcatcacaaa
tttcacaaataaagcatttttttcactgcattctagttgtggtttgtccaaactcatcaatgtatctt
atcatgtctgtataccgtcgacctctagctagagcttggcgtaatcatggtcatagctgtttcctgtg
tgaaattgttatccgctcacaattccacacaacatacgagccggaagcataaagtgtaaagcctgggg
tgcctaatgagtgagctaactcacattaattgcgttgcgctcactgcccgctttccagtcgggaaacc
tgtcgtgccagctgcattaatgaatcggccaacgcgcggggagaggcggtttgcgtattgggcgctct
tccgcttcctcgctcactgactcgctgcgctcggtcgttcggctgcggcgagcggtatcagctcactc
aaaggcggtaatacggttatccacagaatcaggggataacgcaggaaagaacatgtgagcaaaaggcc
agcaaaaggccaggaaccgtaaaaaggccgcgttgctggcgtttttccataggctccgcccccctgac
gagcatcacaaaaatcgacgctcaagtcagaggtggcgaaacccgacaggactataaagataccaggc
gtttccccctggaagctccctcgtgcgctctcctgttccgaccctgccgcttaccggatacctgtccg
cctttctcccttcgggaagcgtggcgctttctcatagctcacgctgtaggtatctcagttcggtgtag
gtcgttcgctccaagctgggctgtgtgcacgaaccccccgttcagcccgaccgctgcgccttatccgg
taactatcgtcttgagtccaacccggtaagacacgacttatcgccactggcagcagccactggtaaca
ggattagcagagcgaggtatgtaggcggtgctacagagttcttgaagtggtggcctaactacggctac
actagaagaacagtatttggtatctgcgctctgctgaagccagttaccttcggaaaaagagttggtag
ctcttgatccggcaaacaaaccaccgctggtagcggtggtttttttgtttgcaagcagcagattacgc
gcagaaaaaaaggatctcaagaagatcctttgatcttttctacggggtctgacgctcagtggaacgaa
aactcacgttaagggattttggtcatgagattatcaaaaaggatcttcacctagatccttttaaatta
aaaatgaagttttaaatcaatctaaagtatatatgagtaaacttggtctgacagttaccaatgcttaa
tcagtgaggcacctatctcagcgatctgtctatttcgttcatccatagttgcctgactccccgtcgtg
tagataactacgatacgggagggcttaccatctggccccagtgctgcaatgataccgcgagacccacg
ctcaccggctccagatttatcagcaataaaccagccagccggaagggccgagcgcagaagtggtcctg
caactttatccgcctccatccagtctattaattgttgccgggaagctagagtaagtagttcgccagtt
aatagtttgcgcaacgttgttgccattgctacaggcatcgtggtgtcacgctcgtcgtttggtatggc
ttcattcagctccggttcccaacgatcaaggcgagttacatgatcccccatgttgtgcaaaaaagcgg
ttagctccttcggtcctccgatcgttgtcagaagtaagttggccgcagtgttatcactcatggttatg
gcagcactgcataattctcttactgtcatgccatccgtaagatgcttttctgtgactggtgagtactc
aaccaagtcattctgagaatagtgtatgcggcgaccgagttgctcttgcccggcgtcaatacgggata
ataccgcgccacatagcagaactttaaaagtgctcatcattggaaaacgttcttcggggcgaaaactc
tcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaa
taagggcgacacggaaatgttgaatactcatactcttcctttttcaatattattgaagcatttatcag
ggttattgtctcatgagcggatacatatttgaatgtatttagaaaaataaacaaataggggttccgcg
cacatttccccgaaaagtgccacctgacgtc
pZV11_SARS-CoV-2-UTR-spike_delta_HexaPro_SPY_tag_S314_RBD_S683
SEQ ID NO: 93
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccgagaataaactagtattcttctggtccccacagactcagagagaaccc
gccaccatgtttgtgttcctggtgctgctgccactggtgtccagccgtggcgtgcctcatatcgtgat
ggtggacgcctacaagcgttacaagagcaacttcagggtccaaccaacagagagcattgtgaggtttc
caaacatcaccaacctgtgtccatttggagaggtgttcaatgccaccaggtttgcctctgtctatgcc
tggaacaggaagaggattagcaactgtgtggctgactactctgtgctctacaactctgcctccttcag
caccttcaagtgttatggagtgagcccaaccaaactgaatgacctgtgtttcaccaatgtctatgctg
actcctttgtgattaggggagatgaggtgagacagattgcccctggacaaacaggcaagattgctgac
tacaactacaaactgcctgatgacttcacaggctgtgtgattgcctggaacagcaacaacctggacag
caaggtgggaggcaactacaactacaggtacagactgttcaggaagagcaacctgaaaccatttgaga
gggacatcagcacagagatttaccaggctggcagcaagccatgtaatggagtggagggcttcaactgt
tactttccactccaatcctatggcttccaaccaaccaatggagtgggctaccaaccatacagggtggt
ggtgctgtcctttgaactgctccatgcccctgccacagtgtgtggaccaaagaagagcaccaacctgg
tgaagaacaagtgtgtgaacttcaacttcaatggactgacaggcacaggagtgctgacagagagcaac
aagaagttcctgccattccaacagtttggcagggacattgctgacaccacagatgctgtgagggaccc
acagaccttggagattctggacatcacaccatgttcctttggaggagtgtctgtgattacacctggca
ccaacaccagcaaccaggtggctgtgctctaccagggcgtgaactgtactgaggtgcctgtggctatc
catgctgaccaacttacaccaacctggagggtctacagcacaggaagcaacgtgttccagaccagggc
tggctgtctgattggagcagagcatgtgaacaactcctatgagtgtgacatcccaattggagcaggca
tctgtgcctcctaccagacccagaccaacagcaggggctctgcatcttgactcgagctggtactgcat
gcacgcaatgctagctgcccctttcccgtcctgggtaccccgagtctcccccgacctcgggtcccagg
tatgctcccacctccacctgccccactcaccacctctgctagttccagacacctcccaagcacgcagc
aatgcagctcaaaacgcttagcctagccacacccccacgggaaacagcagtgattaacctttagcaat
aaacgaaagtttaactaagctatactaaccccagggttggtcaatttcgtgccagccacaccctggag
ctagcaaaccgctgatcagcctcgactgtgccttctagttgccagccatctgttgtttgcccctcccc
cgtgccttccttgaccctggaaggtgccactcccactgtcctttcctaataaaatgaggaaattgcat
cgcattgtctgagtaggtgtcattctattctggggggtggggtggggcaggacagcaagggggaggat
tgggaagacaatagcaggcatgctggggatgcggtgggctctatggcttctgaggcggaaagaaccag
ctggggctctagggggtatccccacgcgccctgtagcggcgcattaagcgcggcgggtgtggtggtta
cgcgcagcgtgaccgctacacttgccagcgccctagcgcccgctcctttcgctttcttcccttccttt
ctcgccacgttcgccggctttccccgtcaagctctaaatcgggggctccctttagggttccgatttag
tgctttacggcacctcgaccccaaaaaacttgattagggtgatggttcacgtagtgggccatcgccct
gatagacggtttttcgccctttgacgttggagtccacgttctttaatagtggactcttgttccaaact
ggaacaacactcaaccctatctcggtctattcttttgatttataagggattttgccgatttcggccta
ttggttaaaaaatgagctgatttaacaaaaatttaacgcgaattaattctgtggaatgtgtgtcagtt
agggtgtggaaagtccccaggctccccagcaggcagaagtatgcaaagcatgcatctcaattagtcag
caaccaggtgtggaaagtccccaggctccccagcaggcagaagtatgcaaagcatgcatctcaattag
tcagcaaccatagtcccgcccctaactccgcccatcccgcccctaactccgcccagttccgcccattc
tccgccccatggctgactaattttttttatttatgcagaggccgaggccgcctctgcctctgagctat
tccagaagtagtgaggaggcttttttggaggcctaggcttttgcaaaaagctcccgggagcttgtata
tccattttcggatctgatcaagagacaggatgaggatcgtttcgcatgattgaacaagatggattgca
cgcaggttctccggccgcttgggtggagaggctattcggctatgactgggcacaacagacaatcggct
gctctgatgccgccgtgttccggctgtcagcgcaggggcgcccggttctttttgtcaagaccgacctg
tccggtgccctgaatgaactgcaggacgaggcagcgcggctatcgtggctggccacgacgggcgttcc
ttgcgcagctgtgctcgacgttgtcactgaagcgggaagggactggctgctattgggcgaagtgccgg
ggcaggatctcctgtcatctcaccttgctcctgccgagaaagtatccatcatggctgatgcaatgcgg
cggctgcatacgcttgatccggctacctgcccattcgaccaccaagcgaaacatcgcatcgagcgagc
acgtactcggatggaagccggtcttgtcgatcaggatgatctggacgaagagcatcaggggctcgcgc
cagccgaactgttcgccaggctcaaggcgcgcatgcccgacggcgaggatctcgtcgtgacccatggc
gatgcctgcttgccgaatatcatggtggaaaatggccgcttttctggattcatcgactgtggccggct
gggtgtggcggaccgctatcaggacatagcgttggctacccgtgatattgctgaagagcttggcggcg
aatgggctgaccgcttcctcgtgctttacggtatcgccgctcccgattcgcagcgcatcgccttctat
cgccttcttgacgagttcttctgagcgggactctggggttcgaaatgaccgaccaagcgacgcccaac
ctgccatcacgagatttcgattccaccgccgccttctatgaaaggttgggcttcggaatcgttttccg
ggacgccggctggatgatcctccagcgcggggatctcatgctggagttcttcgcccaccccaacttgt
ttattgcagcttataatggttacaaataaagcaatagcatcacaaatttcacaaataaagcatttttt
tcactgcattctagttgtggtttgtccaaactcatcaatgtatcttatcatgtctgtataccgtcgac
ctctagctagagcttggcgtaatcatggtcatagctgtttcctgtgtgaaattgttatccgctcacaa
ttccacacaacatacgagccggaagcataaagtgtaaagcctggggtgcctaatgagtgagctaactc
acattaattgcgttgcgctcactgcccgctttccagtcgggaaacctgtcgtgccagctgcattaatg
aatcggccaacgcgcggggagaggcggtttgcgtattgggcgctcttccgcttcctcgctcactgact
cgctgcgctcggtcgttcggctgcggcgagcggtatcagctcactcaaaggcggtaatacggttatcc
acagaatcaggggataacgcaggaaagaacatgtgagcaaaaggccagcaaaaggccaggaaccgtaa
aaaggccgcgttgctggcgtttttccataggctccgcccccctgacgagcatcacaaaaatcgacgct
caagtcagaggtggcgaaacccgacaggactataaagataccaggcgtttccccctggaagctccctc
gtgcgctctcctgttccgaccctgccgcttaccggatacctgtccgcctttctcccttcgggaagcgt
ggcgctttctcatagctcacgctgtaggtatctcagttcggtgtaggtcgttcgctccaagctgggct
gtgtgcacgaaccccccgttcagcccgaccgctgcgccttatccggtaactatcgtcttgagtccaac
ccggtaagacacgacttatcgccactggcagcagccactggtaacaggattagcagagcgaggtatgt
aggcggtgctacagagttcttgaagtggtggcctaactacggctacactagaagaacagtatttggta
tctgcgctctgctgaagccagttaccttcggaaaaagagttggtagctcttgatccggcaaacaaacc
accgctggtagcggtggtttttttgtttgcaagcagcagattacgcgcagaaaaaaaggatctcaaga
agatcctttgatcttttctacggggtctgacgctcagtggaacgaaaactcacgttaagggattttgg
tcatgagattatcaaaaaggatcttcacctagatccttttaaattaaaaatgaagttttaaatcaatc
taaagtatatatgagtaaacttggtctgacagttaccaatgcttaatcagtgaggcacctatctcagc
gatctgtctatttcgttcatccatagttgcctgactccccgtcgtgtagataactacgatacgggagg
gcttaccatctggccccagtgctgcaatgataccgcgagacccacgctcaccggctccagatttatca
gcaataaaccagccagccggaagggccgagcgcagaagtggtcctgcaactttatccgcctccatcca
gtctattaattgttgccgggaagctagagtaagtagttcgccagttaatagtttgcgcaacgttgttg
ccattgctacaggcatcgtggtgtcacgctcgtcgtttggtatggcttcattcagctccggttcccaa
cgatcaaggcgagttacatgatcccccatgttgtgcaaaaaagcggttagctccttcggtcctccgat
cgttgtcagaagtaagttggccgcagtgttatcactcatggttatggcagcactgcataattctctta
ctgtcatgccatccgtaagatgcttttctgtgactggtgagtactcaaccaagtcattctgagaatag
tgtatgcggcgaccgagttgctcttgcccggcgtcaatacgggataataccgcgccacatagcagaac
tttaaaagtgctcatcattggaaaacgttcttcggggcgaaaactctcaaggatcttaccgctgttga
gatccagttcgatgtaacccactcgtgcacccaactgatcttcagcatcttttactttcaccagcgtt
tctgggtgagcaaaaacaggaaggcaaaatgccgcaaaaaagggaataagggcgacacggaaatgttg
aatactcatactcttcctttttcaatattattgaagcatttatcagggttattgtctcatgagcggat
acatatttgaatgtatttagaaaaataaacaaataggggttccgcgcacatttccccgaaaagtgcca
cctgacgtc
pZV12_SARS-CoV-2-UTR-spike_delta_HexaPro_SPY_tag_S314_RBD_S683_
T2A_SARS_RBD_T2A_MERS_RBD
SEQ ID NO: 94
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggctagccaccgagaataaactagtattcttctggtccccacagactcagagagaaccc
gccaccatgtttgtgttcctggtgctgctgccactggtgtccagccgtggcgtgcctcatatcgtgat
ggtggacgcctacaagcgttacaagagcaacttcagggtccaaccaacagagagcattgtgaggtttc
caaacatcaccaacctgtgtccatttggagaggtgttcaatgccaccaggtttgcctctgtctatgcc
tggaacaggaagaggattagcaactgtgtggctgactactctgtgctctacaactctgcctccttcag
caccttcaagtgttatggagtgagcccaaccaaactgaatgacctgtgtttcaccaatgtctatgctg
actcctttgtgattaggggagatgaggtgagacagattgcccctggacaaacaggcaagattgctgac
tacaactacaaactgcctgatgacttcacaggctgtgtgattgcctggaacagcaacaacctggacag
caaggtgggaggcaactacaactacaggtacagactgttcaggaagagcaacctgaaaccatttgaga
gggacatcagcacagagatttaccaggctggcagcaagccatgtaatggagtggagggcttcaactgt
tactttccactccaatcctatggcttccaaccaaccaatggagtgggctaccaaccatacagggtggt
ggtgctgtcctttgaactgctccatgcccctgccacagtgtgtggaccaaagaagagcaccaacctgg
tgaagaacaagtgtgtgaacttcaacttcaatggactgacaggcacaggagtgctgacagagagcaac
aagaagttcctgccattccaacagtttggcagggacattgctgacaccacagatgctgtgagggaccc
acagaccttggagattctggacatcacaccatgttcctttggaggagtgtctgtgattacacctggca
ccaacaccagcaaccaggtggctgtgctctaccagggcgtgaactgtactgaggtgcctgtggctatc
catgctgaccaacttacaccaacctggagggtctacagcacaggaagcaacgtgttccagaccagggc
tggctgtctgattggagcagagcatgtgaacaactcctatgagtgtgacatcccaattggagcaggca
tctgtgcctcctaccagacccagaccaacagcaggggctctgcatctggctccggcgagggcagaggc
agtctgctgacatgcggtgacgtggaagagaatcccggccctatgtttgtgttcctggtgctgctgcc
actggtgtccagccgtggcgtgcctcatatcgtgatggtggacgcctacaagcgttacaagagcaact
tcagggtggtgccatctggagatgtggtgaggtttccaaacatcaccaacctgtgtccatttggagag
gtgttcaatgccaccaagtttccatctgtctatgcctgggagaggaagaagattagcaactgtgtggc
tgactactctgtgctctacaacagcaccttcttcagcaccttcaagtgttatggagtgtctgccacca
aactgaatgacctgtgtttcagcaatgtctatgctgactcctttgtggtgaagggagatgatgtgaga
cagattgcccctggacaaacaggagtgattgctgactacaactacaaactgcctgatgactttatggg
ctgtgtgctggcttggaacaccaggaacattgatgccaccagcacaggcaactacaactacaaataca
gatacctgagacatggcaaactgagaccatttgagagggacatcagcaatgtgccattcagccctgat
ggcaagccatgtactcctcctgccctgaactgttactggccactgaatgactatggcttctacaccac
cacaggcattggctaccaaccatacagggtggtggtgctgtcctttgaactgctgaatgcccctgcca
cagtgtgtggaccaaaaaagagcaccaacctggtgaagaacaagtgtgtgaacttcaacttcaatgga
ctgacaggcacaggagtgctgacagagagcaacaagaagttcctgccattccaacagtttggcaggga
cattgctgacaccacagatgctgtgagggacccacagaccttggagattctggacatcacaccatgtt
cctttggaggagtgtctgtgattacacctggcaccaacaccagcaaccaggtggctgtgctctaccag
ggcgtgaactgtactgaggtgcctgtggctatccatgctgaccaacttacaccaacctggagggtcta
cagcacaggaagcaacgtgttccagaccagggctggctgtctgattggagcagagcatgtgaacaact
cctatgagtgtgacatcccaattggagcaggcatctgtgcctcctaccagacccagaccaacagcagg
ggctctgcatctggctccggcgagggcagaggcagtctgctgacatgcggtgacgtggaagagaatcc
cggccctatgtttgtgttcctggtgctgctgccactggtgtccagccgtggcgtgcctcatatcgtga
tggtggacgcctacaagcgttacaagagcaacttcgaggctaagccatctggctctgtggtggaacag
gctgagggagtggagtgtgacttcagcccactgctgtctggcacacctccacaggtctacaacttcaa
gagactggtgttcaccaactgtaactacaacctgaccaaactgctgtccctgttctctgtgaatgact
tcacttgtagccagattagccctgctgccattgccagcaactgttactcctccctgattctggactac
ttctcctacccactgagtatgaagtctgacctgtctgtgtcctctgctggaccaatcagccagttcaa
ctacaagcagtccttcagcaacccaacttgtctgattctggctacagtgccacacaacctgaccacca
tcaccaagccactgaaatactcctacatcaacaagtgtagcagactgctgtctgatgacaggacagag
gtgccacaactagtgaatgccaaccaatacagcccatgtgtgagcattgtgccaagcacagtgtggga
ggatggagactactacaggaagcaacttagcccattggagggaggaggctggctggtggcatctggca
gcacagtggctatgacagaacaactccaaatgggctttggcatcacagtccaatatggcacagacacc
aactctgtgtgtccaaaattgaagagcaccaacctggtgaagaacaagtgtgtgaacttcaacttcaa
tggactgacaggcacaggagtgctgacagagagcaacaagaagttcctgccattccaacagtttggca
gggacattgctgacaccacagatgctgtgagggacccacagaccttggagattctggacatcacacca
tgttcctttggaggagtgtctgtgattacacctggcaccaacaccagcaaccaggtggctgtgctcta
ccagggcgtgaactgtactgaggtgcctgtggctatccatgctgaccaacttacaccaacctggaggg
tctacagcacaggaagcaacgtgttccagaccagggctggctgtctgattggagcagagcatgtgaac
aactcctatgagtgtgacatcccaattggagcaggcatctgtgcctcctaccagacccagaccaacag
caggggctctgcatcttgactcgagctggtactgcatgcacgcaatgctagctgcccctttcccgtcc
tgggtaccccgagtctcccccgacctcgggtcccaggtatgctcccacctccacctgccccactcacc
acctctgctagttccagacacctcccaagcacgcagcaatgcagctcaaaacgcttagcctagccaca
cccccacgggaaacagcagtgattaacctttagcaataaacgaaagtttaactaagctatactaaccc
cagggttggtcaatttcgtgccagccacaccctggagctagcaaaccgctgatcagcctcgactgtgc
cttctagttgccagccatctgttgtttgcccctcccccgtgccttccttgaccctggaaggtgccact
cccactgtcctttcctaataaaatgaggaaattgcatcgcattgtctgagtaggtgtcattctattct
ggggggtggggtggggcaggacagcaagggggaggattgggaagacaatagcaggcatgctggggatg
cggtgggctctatggcttctgaggcggaaagaaccagctggggctctagggggtatccccacgcgccc
tgtagcggcgcattaagcgcggcgggtgtggtggttacgcgcagcgtgaccgctacacttgccagcgc
cctagcgcccgctcctttcgctttcttcccttcctttctcgccacgttcgccggctttccccgtcaag
ctctaaatcgggggctccctttagggttccgatttagtgctttacggcacctcgaccccaaaaaactt
gattagggtgatggttcacgtagtgggccatcgccctgatagacggtttttcgccctttgacgttgga
gtccacgttctttaatagtggactcttgttccaaactggaacaacactcaaccctatctcggtctatt
cttttgatttataagggattttgccgatttcggcctattggttaaaaaatgagctgatttaacaaaaa
tttaacgcgaattaattctgtggaatgtgtgtcagttagggtgtggaaagtccccaggctccccagca
ggcagaagtatgcaaagcatgcatctcaattagtcagcaaccaggtgtggaaagtccccaggctcccc
agcaggcagaagtatgcaaagcatgcatctcaattagtcagcaaccatagtcccgcccctaactccgc
ccatcccgcccctaactccgcccagttccgcccattctccgccccatggctgactaattttttttatt
tatgcagaggccgaggccgcctctgcctctgagctattccagaagtagtgaggaggcttttttggagg
cctaggcttttgcaaaaagctcccgggagcttgtatatccattttcggatctgatcaagagacaggat
gaggatcgtttcgcatgattgaacaagatggattgcacgcaggttctccggccgcttgggtggagagg
ctattcggctatgactgggcacaacagacaatcggctgctctgatgccgccgtgttccggctgtcagc
gcaggggcgcccggttctttttgtcaagaccgacctgtccggtgccctgaatgaactgcaggacgagg
cagcgcggctatcgtggctggccacgacgggcgttccttgcgcagctgtgctcgacgttgtcactgaa
gcgggaagggactggctgctattgggcgaagtgccggggcaggatctcctgtcatctcaccttgctcc
tgccgagaaagtatccatcatggctgatgcaatgcggcggctgcatacgcttgatccggctacctgcc
cattcgaccaccaagcgaaacatcgcatcgagcgagcacgtactcggatggaagccggtcttgtcgat
caggatgatctggacgaagagcatcaggggctcgcgccagccgaactgttcgccaggctcaaggcgcg
catgcccgacggcgaggatctcgtcgtgacccatggcgatgcctgcttgccgaatatcatggtggaaa
atggccgcttttctggattcatcgactgtggccggctgggtgtggcggaccgctatcaggacatagcg
ttggctacccgtgatattgctgaagagcttggcggcgaatgggctgaccgcttcctcgtgctttacgg
tatcgccgctcccgattcgcagcgcatcgccttctatcgccttcttgacgagttcttctgagcgggac
tctggggttcgaaatgaccgaccaagcgacgcccaacctgccatcacgagatttcgattccaccgccg
ccttctatgaaaggttgggcttcggaatcgttttccgggacgccggctggatgatcctccagcgcggg
gatctcatgctggagttcttcgcccaccccaacttgtttattgcagcttataatggttacaaataaag
caatagcatcacaaatttcacaaataaagcatttttttcactgcattctagttgtggtttgtccaaac
tcatcaatgtatcttatcatgtctgtataccgtcgacctctagctagagcttggcgtaatcatggtca
tagctgtttcctgtgtgaaattgttatccgctcacaattccacacaacatacgagccggaagcataaa
gtgtaaagcctggggtgcctaatgagtgagctaactcacattaattgcgttgcgctcactgcccgctt
tccagtcgggaaacctgtcgtgccagctgcattaatgaatcggccaacgcgcggggagaggcggtttg
cgtattgggcgctcttccgcttcctcgctcactgactcgctgcgctcggtcgttcggctgcggcgagc
ggtatcagctcactcaaaggcggtaatacggttatccacagaatcaggggataacgcaggaaagaaca
tgtgagcaaaaggccagcaaaaggccaggaaccgtaaaaaggccgcgttgctggcgtttttccatagg
ctccgcccccctgacgagcatcacaaaaatcgacgctcaagtcagaggtggcgaaacccgacaggact
ataaagataccaggcgtttccccctggaagctccctcgtgcgctctcctgttccgaccctgccgctta
ccggatacctgtccgcctttctcccttcgggaagcgtggcgctttctcatagctcacgctgtaggtat
ctcagttcggtgtaggtcgttcgctccaagctgggctgtgtgcacgaaccccccgttcagcccgaccg
ctgcgccttatccggtaactatcgtcttgagtccaacccggtaagacacgacttatcgccactggcag
cagccactggtaacaggattagcagagcgaggtatgtaggcggtgctacagagttcttgaagtggtgg
cctaactacggctacactagaagaacagtatttggtatctgcgctctgctgaagccagttaccttcgg
aaaaagagttggtagctcttgatccggcaaacaaaccaccgctggtagcggtggtttttttgtttgca
agcagcagattacgcgcagaaaaaaaggatctcaagaagatcctttgatcttttctacggggtctgac
gctcagtggaacgaaaactcacgttaagggattttggtcatgagattatcaaaaaggatcttcaccta
gatccttttaaattaaaaatgaagttttaaatcaatctaaagtatatatgagtaaacttggtctgaca
gttaccaatgcttaatcagtgaggcacctatctcagcgatctgtctatttcgttcatccatagttgcc
tgactccccgtcgtgtagataactacgatacgggagggcttaccatctggccccagtgctgcaatgat
accgcgagacccacgctcaccggctccagatttatcagcaataaaccagccagccggaagggccgagc
gcagaagtggtcctgcaactttatccgcctccatccagtctattaattgttgccgggaagctagagta
agtagttcgccagttaatagtttgcgcaacgttgttgccattgctacaggcatcgtggtgtcacgctc
gtcgtttggtatggcttcattcagctccggttcccaacgatcaaggcgagttacatgatcccccatgt
tgtgcaaaaaagcggttagctccttcggtcctccgatcgttgtcagaagtaagttggccgcagtgtta
tcactcatggttatggcagcactgcataattctcttactgtcatgccatccgtaagatgcttttctgt
gactggtgagtactcaaccaagtcattctgagaatagtgtatgcggcgaccgagttgctcttgcccgg
cgtcaatacgggataataccgcgccacatagcagaactttaaaagtgctcatcattggaaaacgttct
tcggggcgaaaactctcaaggatcttaccgctgttgagatccagttcgatgtaacccactcgtgcacc
caactgatcttcagcatcttttactttcaccagcgtttctgggtgagcaaaaacaggaaggcaaaatg
ccgcaaaaaagggaataagggcgacacggaaatgttgaatactcatactcttcctttttcaatattat
tgaagcatttatcagggttattgtctcatgagcggatacatatttgaatgtatttagaaaaataaaca
aataggggttccgcgcacatttccccgaaaagtgccacctgacgtc
pMD119_pcDNA3.1_ecto-SARS-CoV-2_spike_VG40589-UT
SEQ ID NO: 95
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCAAGATTGCTGACTACAACTACAAACTGCCTGAT
GACTTCACAGGCTGTGTGATTGCCTGGAACAGCAACAACCTGGACAGCAAGGTGGGAGGCAACTACAA
CTACCTCTACAGACTGTTCAGGAAGAGCAACCTGAAACCATTTGAGAGGGACATCAGCACAGAGATTT
ACCAGGCTGGCAGCACACCATGTAATGGAGTGGAGGGCTTCAACTGTTACTTTCCACTCCAATCCTAT
GGCTTCCAACCAACCAATGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAAtaagtttaaacccgctgatcagcctcgactgtgccttctagttgcc
agccatctgttgtttgcccctcccccgtgccttccttgaccctggaaggtgccactcccactgtcctt
tcctaataaaatgaggaaattgcatcgcattgtctgagtaggtgtcattctattctggggggtggggt
ggggcaggacagcaagggggaggattgggaagacaatagcaggcatgctggggatgcggtgggctcta
tggcttctgaggcggaaagaaccagctggggctctagggggtatccccacgcgccctgtagcggcgca
ttaagcgcggcgggtgtggtggttacgcgcagcgtgaccgctacacttgccagcgccctagcgcccgc
tcctttcgctttcttcccttcctttctcgccacgttcgccggctttccccgtcaagctctaaatcggg
ggctccctttagggttccgatttagtgctttacggcacctcgaccccaaaaaacttgattagggtgat
ggttcacgtagtgggccatcgccctgatagacggtttttcgccctttgacgttggagtccacgttctt
taatagtggactcttgttccaaactggaacaacactcaaccctatctcggtctattcttttgatttat
aagggattttgccgatttcggcctattggttaaaaaatgagctgatttaacaaaaatttaacgcgaat
taattctgtggaatgtgtgtcagttagggtgtggaaagtccccaggctccccagcaggcagaagtatg
caaagcatgcatctcaattagtcagcaaccaggtgtggaaagtccccaggctccccagcaggcagaag
tatgcaaagcatgcatctcaattagtcagcaaccatagtcccgcccctaactccgcccatcccgcccc
taactccgcccagttccgcccattctccgccccatggctgactaattttttttatttatgcagaggcc
gaggccgcctctgcctctgagctattccagaagtagtgaggaggcttttttggaggcctaggcttttg
caaaaagctcccgggagcttgtatatccattttcggatctgatcaagagacaggatgaggatcgtttc
gcatgattgaacaagatggattgcacgcaggttctccggccgcttgggtggagaggctattcggctat
gactgggcacaacagacaatcggctgctctgatgccgccgtgttccggctgtcagcgcaggggcgccc
ggttctttttgtcaagaccgacctgtccggtgccctgaatgaactgcaggacgaggcagcgcggctat
cgtggctggccacgacgggcgttccttgcgcagctgtgctcgacgttgtcactgaagcgggaagggac
tggctgctattgggcgaagtgccggggcaggatctcctgtcatctcaccttgctcctgccgagaaagt
atccatcatggctgatgcaatgcggcggctgcatacgcttgatccggctacctgcccattcgaccacc
aagcgaaacatcgcatcgagcgagcacgtactcggatggaagccggtcttgtcgatcaggatgatctg
gacgaagagcatcaggggctcgcgccagccgaactgttcgccaggctcaaggcgcgcatgcccgacgg
cgaggatctcgtcgtgacccatggcgatgcctgcttgccgaatatcatggtggaaaatggccgctttt
ctggattcatcgactgtggccggctgggtgtggcggaccgctatcaggacatagcgttggctacccgt
gatattgctgaagagcttggcggcgaatgggctgaccgcttcctcgtgctttacggtatcgccgctcc
cgattcgcagcgcatcgccttctatcgccttcttgacgagttcttctgagcgggactctggggttcga
aatgaccgaccaagcgacgcccaacctgccatcacgagatttcgattccaccgccgccttctatgaaa
ggttgggcttcggaatcgttttccgggacgccggctggatgatcctccagcgcggggatctcatgctg
gagttcttcgcccaccccaacttgtttattgcagcttataatggttacaaataaagcaatagcatcac
aaatttcacaaataaagcatttttttcactgcattctagttgtggtttgtccaaactcatcaatgtat
cttatcatgtctgtataccgtcgacctctagctagagcttggcgtaatcatggtcatagctgtttcct
gtgtgaaattgttatccgctcacaattccacacaacatacgagccggaagcataaagtgtaaagcctg
gggtgcctaatgagtgagctaactcacattaattgcgttgcgctcactgcccgctttccagtcgggaa
acctgtcgtgccagctgcattaatgaatcggccaacgcgcggggagaggcggtttgcgtattgggcgc
tcttccgcttcctcgctcactgactcgctgcgctcggtcgttcggctgcggcgagcggtatcagctca
ctcaaaggcggtaatacggttatccacagaatcaggggataacgcaggaaagaacatgtgagcaaaag
gccagcaaaaggccaggaaccgtaaaaaggccgcgttgctggcgtttttccataggctccgcccccct
gacgagcatcacaaaaatcgacgctcaagtcagaggtggcgaaacccgacaggactataaagatacca
ggcgtttccccctggaagctccctcgtgcgctctcctgttccgaccctgccgcttaccggatacctgt
ccgcctttctcccttcgggaagcgtggcgctttctcatagctcacgctgtaggtatctcagttcggtg
taggtcgttcgctccaagctgggctgtgtgcacgaaccccccgttcagcccgaccgctgcgccttatc
cggtaactatcgtcttgagtccaacccggtaagacacgacttatcgccactggcagcagccactggta
acaggattagcagagcgaggtatgtaggcggtgctacagagttcttgaagtggtggcctaactacggc
tacactagaagaacagtatttggtatctgcgctctgctgaagccagttaccttcggaaaaagagttgg
tagctcttgatccggcaaacaaaccaccgctggtagcggtttttttgtttgcaagcagcagattacgc
gcagaaaaaaaggatctcaagaagatcctttgatcttttctacggggtctgacgctcagtggaacgaa
aactcacgttaagggattttggtcatgagattatcaaaaaggatcttcacctagatccttttaaatta
aaaatgaagttttaaatcaatctaaagtatatatgagtaaacttggtctgacagttaccaatgcttaa
tcagtgaggcacctatctcagcgatctgtctatttcgttcatccatagttgcctgactccccgtcgtg
tagataactacgatacgggagggcttaccatctggccccagtgctgcaatgataccgcgagacccacg
ctcaccggctccagatttatcagcaataaaccagccagccggaagggccgagcgcagaagtggtcctg
caactttatccgcctccatccagtctattaattgttgccgggaagctagagtaagtagttcgccagtt
aatagtttgcgcaacgttgttgccattgctacaggcatcgtggtgtcacgctcgtcgtttggtatggc
ttcattcagctccggttcccaacgatcaaggcgagttacatgatcccccatgttgtgcaaaaaagcgg
ttagctccttcggtcctccgatcgttgtcagaagtaagttggccgcagtgttatcactcatggttatg
gcagcactgcataattctcttactgtcatgccatccgtaagatgcttttctgtgactggtgagtactc
aaccaagtcattctgagaatagtgtatgcggcgaccgagttgctcttgcccggcgtcaatacgggata
ataccgcgccacatagcagaactttaaaagtgctcatcattggaaaacgttcttcggggcgaaaactc
tcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaa
taagggcgacacggaaatgttgaatactcatactcttcctttttcaatattattgaagcatttatcag
ggttattgtctcatgagcggatacatatttgaatgtatttagaaaaataaacaaataggggttccgcg
cacatttccccgaaaagtgccacctgacgtc
pMD120_pcDNA3.1_SARS-CoV-2_spike_VG40589-UT
SEQ ID NO: 96
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCAAGATTGCTGACTACAACTACAAACTGCCTGAT
GACTTCACAGGCTGTGTGATTGCCTGGAACAGCAACAACCTGGACAGCAAGGTGGGAGGCAACTACAA
CTACCTCTACAGACTGTTCAGGAAGAGCAACCTGAAACCATTTGAGAGGGACATCAGCACAGAGATTT
ACCAGGCTGGCAGCACACCATGTAATGGAGTGGAGGGCTTCAACTGTTACTTTCCACTCCAATCCTAT
GGCTTCCAACCAACCAATGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTAAGTTTGATGAGGATGACTCTGAACCTGTGCTGAA
AGGAGTGAAACTGCACTACACCTGAgtttaaacccgctgatcagcctcgactgtgccttctagttgcc
agccatctgttgtttgcccctcccccgtgccttccttgaccctggaaggtgccactcccactgtcctt
tcctaataaaatgaggaaattgcatcgcattgtctgagtaggtgtcattctattctggggggtggggt
ggggcaggacagcaagggggaggattgggaagacaatagcaggcatgctggggatgcggtgggctcta
tggcttctgaggcggaaagaaccagctggggctctagggggtatccccacgcgccctgtagcggcgca
ttaagcgcggcgggtgtggtggttacgcgcagcgtgaccgctacacttgccagcgccctagcgcccgc
tcctttcgctttcttcccttcctttctcgccacgttcgccggctttccccgtcaagctctaaatcggg
ggctccctttagggttccgatttagtgctttacggcacctcgaccccaaaaaacttgattagggtgat
ggttcacgtagtgggccatcgccctgatagacggtttttcgccctttgacgttggagtccacgttctt
taatagtggactcttgttccaaactggaacaacactcaaccctatctcggtctattcttttgatttat
aagggattttgccgatttcggcctattggttaaaaaatgagctgatttaacaaaaatttaacgcgaat
taattctgtggaatgtgtgtcagttagggtgtggaaagtccccaggctccccagcaggcagaagtatg
caaagcatgcatctcaattagtcagcaaccaggtgtggaaagtccccaggctccccagcaggcagaag
tatgcaaagcatgcatctcaattagtcagcaaccatagtcccgcccctaactccgcccatcccgcccc
taactccgcccagttccgcccattctccgccccatggctgactaattttttttatttatgcagaggcc
gaggccgcctctgcctctgagctattccagaagtagtgaggaggcttttttggaggcctaggcttttg
caaaaagctcccgggagcttgtatatccattttcggatctgatcaagagacaggatgaggatcgtttc
gcatgattgaacaagatggattgcacgcaggttctccggccgcttgggtggagaggctattcggctat
gactgggcacaacagacaatcggctgctctgatgccgccgtgttccggctgtcagcgcaggggcgccc
ggttctttttgtcaagaccgacctgtccggtgccctgaatgaactgcaggacgaggcagcgcggctat
cgtggctggccacgacgggcgttccttgcgcagctgtgctcgacgttgtcactgaagcgggaagggac
tggctgctattgggcgaagtgccggggcaggatctcctgtcatctcaccttgctcctgccgagaaagt
atccatcatggctgatgcaatgcggcggctgcatacgcttgatccggctacctgcccattcgaccacc
aagcgaaacatcgcatcgagcgagcacgtactcggatggaagccggtcttgtcgatcaggatgatctg
gacgaagagcatcaggggctcgcgccagccgaactgttcgccaggctcaaggcgcgcatgcccgacgg
cgaggatctcgtcgtgacccatggcgatgcctgcttgccgaatatcatggtggaaaatggccgctttt
ctggattcatcgactgtggccggctgggtgtggcggaccgctatcaggacatagcgttggctacccgt
gatattgctgaagagcttggcggcgaatgggctgaccgcttcctcgtgctttacggtatcgccgctcc
cgattcgcagcgcatcgccttctatcgccttcttgacgagttcttctgagcgggactctggggttcga
aatgaccgaccaagcgacgcccaacctgccatcacgagatttcgattccaccgccgccttctatgaaa
ggttgggcttcggaatcgttttccgggacgccggctggatgatcctccagcgcggggatctcatgctg
gagttcttcgcccaccccaacttgtttattgcagcttataatggttacaaataaagcaatagcatcac
aaatttcacaaataaagcatttttttcactgcattctagttgtggtttgtccaaactcatcaatgtat
cttatcatgtctgtataccgtcgacctctagctagagcttggcgtaatcatggtcatagctgtttcct
gtgtgaaattgttatccgctcacaattccacacaacatacgagccggaagcataaagtgtaaagcctg
gggtgcctaatgagtgagctaactcacattaattgcgttgcgctcactgcccgctttccagtcgggaa
acctgtcgtgccagctgcattaatgaatcggccaacgcgcggggagaggcggtttgcgtattgggcgc
tcttccgcttcctcgctcactgactcgctgcgctcggtcgttcggctgcggcgagcggtatcagctca
ctcaaaggcggtaatacggttatccacagaatcaggggataacgcaggaaagaacatgtgagcaaaag
gccagcaaaaggccaggaaccgtaaaaaggccgcgttgctggcgtttttccataggctccgcccccct
gacgagcatcacaaaaatcgacgctcaagtcagaggtggcgaaacccgacaggactataaagatacca
ggcgtttccccctggaagctccctcgtgcgctctcctgttccgaccctgccgcttaccggatacctgt
ccgcctttctcccttcgggaagcgtggcgctttctcatagctcacgctgtaggtatctcagttcggtg
taggtcgttcgctccaagctgggctgtgtgcacgaaccccccgttcagcccgaccgctgcgccttatc
cggtaactatcgtcttgagtccaacccggtaagacacgacttatcgccactggcagcagccactggta
acaggattagcagagcgaggtatgtaggcggtgctacagagttcttgaagtggtggcctaactacggc
tacactagaagaacagtatttggtatctgcgctctgctgaagccagttaccttcggaaaaagagttgg
tagctcttgatccggcaaacaaaccaccgctggtagcggtttttttgtttgcaagcagcagattacgc
gcagaaaaaaaggatctcaagaagatcctttgatcttttctacggggtctgacgctcagtggaacgaa
aactcacgttaagggattttggtcatgagattatcaaaaaggatcttcacctagatccttttaaatta
aaaatgaagttttaaatcaatctaaagtatatatgagtaaacttggtctgacagttaccaatgcttaa
tcagtgaggcacctatctcagcgatctgtctatttcgttcatccatagttgcctgactccccgtcgtg
tagataactacgatacgggagggcttaccatctggccccagtgctgcaatgataccgcgagacccacg
ctcaccggctccagatttatcagcaataaaccagccagccggaagggccgagcgcagaagtggtcctg
caactttatccgcctccatccagtctattaattgttgccgggaagctagagtaagtagttcgccagtt
aatagtttgcgcaacgttgttgccattgctacaggcatcgtggtgtcacgctcgtcgtttggtatggc
ttcattcagctccggttcccaacgatcaaggcgagttacatgatcccccatgttgtgcaaaaaagcgg
ttagctccttcggtcctccgatcgttgtcagaagtaagttggccgcagtgttatcactcatggttatg
gcagcactgcataattctcttactgtcatgccatccgtaagatgcttttctgtgactggtgagtactc
aaccaagtcattctgagaatagtgtatgcggcgaccgagttgctcttgcccggcgtcaatacgggata
ataccgcgccacatagcagaactttaaaagtgctcatcattggaaaacgttcttcggggcgaaaactc
tcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaa
taagggcgacacggaaatgttgaatactcatactcttcctttttcaatattattgaagcatttatcag
ggttattgtctcatgagcggatacatatttgaatgtatttagaaaaataaacaaataggggttccgcg
cacatttccccgaaaagtgccacctgacgtc
pMD121_pcDNA3_SARS-CoV-2_spike_mut_v1
SEQ ID NO: 97
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCAAGATTGCTGACTACAACTACAAACTGCCTGAT
GACTTCACAGGCTGTGTGATTGCCTGGAACAGCgccAACCTGGACAGCAAGGTGGGAGGCAACgccAA
CTACCTCTACAGACTGTTCAGGAAGAGCAACCTGAAACCATTTGAGAGGGACATCAGCACAGAGATTT
ACCAGGCTGGCAGCACACCATGTAATGGAGTGGAGGGCTTCAACTGTTACTTTCCACTCCAATCCTAT
GGCTTCgccCCAgctgccGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTAAGTTTGATGAGGATGACTCTGAACCTGTGCTGAA
AGGAGTGAAACTGCACTACACCTGAgtttaaacccgctgatcagcctcgactgtgccttctagttgcc
agccatctgttgtttgcccctcccccgtgccttccttgaccctggaaggtgccactcccactgtcctt
tcctaataaaatgaggaaattgcatcgcattgtctgagtaggtgtcattctattctggggggtggggt
ggggcaggacagcaagggggaggattgggaagacaatagcaggcatgctggggatgcggtgggctcta
tggcttctgaggcggaaagaaccagctggggctctagggggtatccccacgcgccctgtagcggcgca
ttaagcgcggcgggtgtggtggttacgcgcagcgtgaccgctacacttgccagcgccctagcgcccgc
tcctttcgctttcttcccttcctttctcgccacgttcgccggctttccccgtcaagctctaaatcggg
ggctccctttagggttccgatttagtgctttacggcacctcgaccccaaaaaacttgattagggtgat
ggttcacgtagtgggccatcgccctgatagacggtttttcgccctttgacgttggagtccacgttctt
taatagtggactcttgttccaaactggaacaacactcaaccctatctcggtctattcttttgatttat
aagggattttgccgatttcggcctattggttaaaaaatgagctgatttaacaaaaatttaacgcgaat
taattctgtggaatgtgtgtcagttagggtgtggaaagtccccaggctccccagcaggcagaagtatg
caaagcatgcatctcaattagtcagcaaccaggtgtggaaagtccccaggctccccagcaggcagaag
tatgcaaagcatgcatctcaattagtcagcaaccatagtcccgcccctaactccgcccatcccgcccc
taactccgcccagttccgcccattctccgccccatggctgactaattttttttatttatgcagaggcc
gaggccgcctctgcctctgagctattccagaagtagtgaggaggcttttttggaggcctaggcttttg
caaaaagctcccgggagcttgtatatccattttcggatctgatcaagagacaggatgaggatcgtttc
gcatgattgaacaagatggattgcacgcaggttctccggccgcttgggtggagaggctattcggctat
gactgggcacaacagacaatcggctgctctgatgccgccgtgttccggctgtcagcgcaggggcgccc
ggttctttttgtcaagaccgacctgtccggtgccctgaatgaactgcaggacgaggcagcgcggctat
cgtggctggccacgacgggcgttccttgcgcagctgtgctcgacgttgtcactgaagcgggaagggac
tggctgctattgggcgaagtgccggggcaggatctcctgtcatctcaccttgctcctgccgagaaagt
atccatcatggctgatgcaatgcggcggctgcatacgcttgatccggctacctgcccattcgaccacc
aagcgaaacatcgcatcgagcgagcacgtactcggatggaagccggtcttgtcgatcaggatgatctg
gacgaagagcatcaggggctcgcgccagccgaactgttcgccaggctcaaggcgcgcatgcccgacgg
cgaggatctcgtcgtgacccatggcgatgcctgcttgccgaatatcatggtggaaaatggccgctttt
ctggattcatcgactgtggccggctgggtgtggcggaccgctatcaggacatagcgttggctacccgt
gatattgctgaagagcttggcggcgaatgggctgaccgcttcctcgtgctttacggtatcgccgctcc
cgattcgcagcgcatcgccttctatcgccttcttgacgagttcttctgagcgggactctggggttcga
aatgaccgaccaagcgacgcccaacctgccatcacgagatttcgattccaccgccgccttctatgaaa
ggttgggcttcggaatcgttttccgggacgccggctggatgatcctccagcgcggggatctcatgctg
gagttcttcgcccaccccaacttgtttattgcagcttataatggttacaaataaagcaatagcatcac
aaatttcacaaataaagcatttttttcactgcattctagttgtggtttgtccaaactcatcaatgtat
cttatcatgtctgtataccgtcgacctctagctagagcttggcgtaatcatggtcatagctgtttcct
gtgtgaaattgttatccgctcacaattccacacaacatacgagccggaagcataaagtgtaaagcctg
gggtgcctaatgagtgagctaactcacattaattgcgttgcgctcactgcccgctttccagtcgggaa
acctgtcgtgccagctgcattaatgaatcggccaacgcgcggggagaggcggtttgcgtattgggcgc
tcttccgcttcctcgctcactgactcgctgcgctcggtcgttcggctgcggcgagcggtatcagctca
ctcaaaggcggtaatacggttatccacagaatcaggggataacgcaggaaagaacatgtgagcaaaag
gccagcaaaaggccaggaaccgtaaaaaggccgcgttgctggcgtttttccataggctccgcccccct
gacgagcatcacaaaaatcgacgctcaagtcagaggtggcgaaacccgacaggactataaagatacca
ggcgtttccccctggaagctccctcgtgcgctctcctgttccgaccctgccgcttaccggatacctgt
ccgcctttctcccttcgggaagcgtggcgctttctcatagctcacgctgtaggtatctcagttcggtg
taggtcgttcgctccaagctgggctgtgtgcacgaaccccccgttcagcccgaccgctgcgccttatc
cggtaactatcgtcttgagtccaacccggtaagacacgacttatcgccactggcagcagccactggta
acaggattagcagagcgaggtatgtaggcggtgctacagagttcttgaagtggtggcctaactacggc
tacactagaagaacagtatttggtatctgcgctctgctgaagccagttaccttcggaaaaagagttgg
tagctcttgatccggcaaacaaaccaccgctggtagcggtttttttgtttgcaagcagcagattacgc
gcagaaaaaaaggatctcaagaagatcctttgatcttttctacggggtctgacgctcagtggaacgaa
aactcacgttaagggattttggtcatgagattatcaaaaaggatcttcacctagatccttttaaatta
aaaatgaagttttaaatcaatctaaagtatatatgagtaaacttggtctgacagttaccaatgcttaa
tcagtgaggcacctatctcagcgatctgtctatttcgttcatccatagttgcctgactccccgtcgtg
tagataactacgatacgggagggcttaccatctggccccagtgctgcaatgataccgcgagacccacg
ctcaccggctccagatttatcagcaataaaccagccagccggaagggccgagcgcagaagtggtcctg
caactttatccgcctccatccagtctattaattgttgccgggaagctagagtaagtagttcgccagtt
aatagtttgcgcaacgttgttgccattgctacaggcatcgtggtgtcacgctcgtcgtttggtatggc
ttcattcagctccggttcccaacgatcaaggcgagttacatgatcccccatgttgtgcaaaaaagcgg
ttagctccttcggtcctccgatcgttgtcagaagtaagttggccgcagtgttatcactcatggttatg
gcagcactgcataattctcttactgtcatgccatccgtaagatgcttttctgtgactggtgagtactc
aaccaagtcattctgagaatagtgtatgcggcgaccgagttgctcttgcccggcgtcaatacgggata
ataccgcgccacatagcagaactttaaaagtgctcatcattggaaaacgttcttcggggcgaaaactc
tcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaa
taagggcgacacggaaatgttgaatactcatactcttcctttttcaatattattgaagcatttatcag
ggttattgtctcatgagcggatacatatttgaatgtatttagaaaaataaacaaataggggttccgcg
cacatttccccgaaaagtgccacctgacgtc
pMD122_pcDNA3_SARS-CoV-2_spike_mut_v2 copy
SEQ ID NO: 98
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCgccATTGCTGACTACAACTACAAACTGCCTGAT
GACTTCACAGGCTGTGTGATTGCCTGGAACAGCAACAACCTGGACAGCAAGGTGGGAGGCAACTACAA
CTACCTCTACAGAgccgctAGGAAGAGCAACCTGAAACCATTTGAGAGGGACATCAGCACAGAGATTg
ccCAGGCTGGCAGCACACCATGTAATGGAGTGGAGGGCTTCAACTGTTACTTTCCACTCgccTCCTAT
GGCTTCCAACCAACCAATGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACgccAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTAAGTTTGATGAGGATGACTCTGAACCTGTGCTGAA
AGGAGTGAAACTGCACTACACCTGAgtttaaacccgctgatcagcctcgactgtgccttctagttgcc
agccatctgttgtttgcccctcccccgtgccttccttgaccctggaaggtgccactcccactgtcctt
tcctaataaaatgaggaaattgcatcgcattgtctgagtaggtgtcattctattctggggggtggggt
ggggcaggacagcaagggggaggattgggaagacaatagcaggcatgctggggatgcggtgggctcta
tggcttctgaggcggaaagaaccagctggggctctagggggtatccccacgcgccctgtagcggcgca
ttaagcgcggcgggtgtggtggttacgcgcagcgtgaccgctacacttgccagcgccctagcgcccgc
tcctttcgctttcttcccttcctttctcgccacgttcgccggctttccccgtcaagctctaaatcggg
ggctccctttagggttccgatttagtgctttacggcacctcgaccccaaaaaacttgattagggtgat
ggttcacgtagtgggccatcgccctgatagacggtttttcgccctttgacgttggagtccacgttctt
taatagtggactcttgttccaaactggaacaacactcaaccctatctcggtctattcttttgatttat
aagggattttgccgatttcggcctattggttaaaaaatgagctgatttaacaaaaatttaacgcgaat
taattctgtggaatgtgtgtcagttagggtgtggaaagtccccaggctccccagcaggcagaagtatg
caaagcatgcatctcaattagtcagcaaccaggtgtggaaagtccccaggctccccagcaggcagaag
tatgcaaagcatgcatctcaattagtcagcaaccatagtcccgcccctaactccgcccatcccgcccc
taactccgcccagttccgcccattctccgccccatggctgactaattttttttatttatgcagaggcc
gaggccgcctctgcctctgagctattccagaagtagtgaggaggcttttttggaggcctaggcttttg
caaaaagctcccgggagcttgtatatccattttcggatctgatcaagagacaggatgaggatcgtttc
gcatgattgaacaagatggattgcacgcaggttctccggccgcttgggtggagaggctattcggctat
gactgggcacaacagacaatcggctgctctgatgccgccgtgttccggctgtcagcgcaggggcgccc
ggttctttttgtcaagaccgacctgtccggtgccctgaatgaactgcaggacgaggcagcgcggctat
cgtggctggccacgacgggcgttccttgcgcagctgtgctcgacgttgtcactgaagcgggaagggac
tggctgctattgggcgaagtgccggggcaggatctcctgtcatctcaccttgctcctgccgagaaagt
atccatcatggctgatgcaatgcggcggctgcatacgcttgatccggctacctgcccattcgaccacc
aagcgaaacatcgcatcgagcgagcacgtactcggatggaagccggtcttgtcgatcaggatgatctg
gacgaagagcatcaggggctcgcgccagccgaactgttcgccaggctcaaggcgcgcatgcccgacgg
cgaggatctcgtcgtgacccatggcgatgcctgcttgccgaatatcatggtggaaaatggccgctttt
ctggattcatcgactgtggccggctgggtgtggcggaccgctatcaggacatagcgttggctacccgt
gatattgctgaagagcttggcggcgaatgggctgaccgcttcctcgtgctttacggtatcgccgctcc
cgattcgcagcgcatcgccttctatcgccttcttgacgagttcttctgagcgggactctggggttcga
aatgaccgaccaagcgacgcccaacctgccatcacgagatttcgattccaccgccgccttctatgaaa
ggttgggcttcggaatcgttttccgggacgccggctggatgatcctccagcgcggggatctcatgctg
gagttcttcgcccaccccaacttgtttattgcagcttataatggttacaaataaagcaatagcatcac
aaatttcacaaataaagcatttttttcactgcattctagttgtggtttgtccaaactcatcaatgtat
cttatcatgtctgtataccgtcgacctctagctagagcttggcgtaatcatggtcatagctgtttcct
gtgtgaaattgttatccgctcacaattccacacaacatacgagccggaagcataaagtgtaaagcctg
gggtgcctaatgagtgagctaactcacattaattgcgttgcgctcactgcccgctttccagtcgggaa
acctgtcgtgccagctgcattaatgaatcggccaacgcgcggggagaggcggtttgcgtattgggcgc
tcttccgcttcctcgctcactgactcgctgcgctcggtcgttcggctgcggcgagcggtatcagctca
ctcaaaggcggtaatacggttatccacagaatcaggggataacgcaggaaagaacatgtgagcaaaag
gccagcaaaaggccaggaaccgtaaaaaggccgcgttgctggcgtttttccataggctccgcccccct
gacgagcatcacaaaaatcgacgctcaagtcagaggtggcgaaacccgacaggactataaagatacca
ggcgtttccccctggaagctccctcgtgcgctctcctgttccgaccctgccgcttaccggatacctgt
ccgcctttctcccttcgggaagcgtggcgctttctcatagctcacgctgtaggtatctcagttcggtg
taggtcgttcgctccaagctgggctgtgtgcacgaaccccccgttcagcccgaccgctgcgccttatc
cggtaactatcgtcttgagtccaacccggtaagacacgacttatcgccactggcagcagccactggta
acaggattagcagagcgaggtatgtaggcggtgctacagagttcttgaagtggtggcctaactacggc
tacactagaagaacagtatttggtatctgcgctctgctgaagccagttaccttcggaaaaagagttgg
tagctcttgatccggcaaacaaaccaccgctggtagcggtttttttgtttgcaagcagcagattacgc
gcagaaaaaaaggatctcaagaagatcctttgatcttttctacggggtctgacgctcagtggaacgaa
aactcacgttaagggattttggtcatgagattatcaaaaaggatcttcacctagatccttttaaatta
aaaatgaagttttaaatcaatctaaagtatatatgagtaaacttggtctgacagttaccaatgcttaa
tcagtgaggcacctatctcagcgatctgtctatttcgttcatccatagttgcctgactccccgtcgtg
tagataactacgatacgggagggcttaccatctggccccagtgctgcaatgataccgcgagacccacg
ctcaccggctccagatttatcagcaataaaccagccagccggaagggccgagcgcagaagtggtcctg
caactttatccgcctccatccagtctattaattgttgccgggaagctagagtaagtagttcgccagtt
aatagtttgcgcaacgttgttgccattgctacaggcatcgtggtgtcacgctcgtcgtttggtatggc
ttcattcagctccggttcccaacgatcaaggcgagttacatgatcccccatgttgtgcaaaaaagcgg
ttagctccttcggtcctccgatcgttgtcagaagtaagttggccgcagtgttatcactcatggttatg
gcagcactgcataattctcttactgtcatgccatccgtaagatgcttttctgtgactggtgagtactc
aaccaagtcattctgagaatagtgtatgcggcgaccgagttgctcttgcccggcgtcaatacgggata
ataccgcgccacatagcagaactttaaaagtgctcatcattggaaaacgttcttcggggcgaaaactc
tcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaa
taagggcgacacggaaatgttgaatactcatactcttcctttttcaatattattgaagcatttatcag
ggttattgtctcatgagcggatacatatttgaatgtatttagaaaaataaacaaataggggttccgcg
cacatttccccgaaaagtgccacctgacgtc
pMD122_pcDNA3_SARS-CoV-2_spike_mut_v2
SEQ ID NO: 99
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCgccATTGCTGACTACAACTACAAACTGCCTGAT
GACTTCACAGGCTGTGTGATTGCCTGGAACAGCAACAACCTGGACAGCAAGGTGGGAGGCAACTACAA
CTACCTCTACAGAgccgctAGGAAGAGCAACCTGAAACCATTTGAGAGGGACATCAGCACAGAGATTg
ccCAGGCTGGCAGCACACCATGTAATGGAGTGGAGGGCTTCAACTGTTACTTTCCACTCgccTCCTAT
GGCTTCCAACCAACCAATGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACgccAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTAAGTTTGATGAGGATGACTCTGAACCTGTGCTGAA
AGGAGTGAAACTGCACTACACCTGAgtttaaacccgctgatcagcctcgactgtgccttctagttgcc
agccatctgttgtttgcccctcccccgtgccttccttgaccctggaaggtgccactcccactgtcctt
tcctaataaaatgaggaaattgcatcgcattgtctgagtaggtgtcattctattctggggggtggggt
ggggcaggacagcaagggggaggattgggaagacaatagcaggcatgctggggatgcggtgggctcta
tggcttctgaggcggaaagaaccagctggggctctagggggtatccccacgcgccctgtagcggcgca
ttaagcgcggcgggtgtggtggttacgcgcagcgtgaccgctacacttgccagcgccctagcgcccgc
tcctttcgctttcttcccttcctttctcgccacgttcgccggctttccccgtcaagctctaaatcggg
ggctccctttagggttccgatttagtgctttacggcacctcgaccccaaaaaacttgattagggtgat
ggttcacgtagtgggccatcgccctgatagacggtttttcgccctttgacgttggagtccacgttctt
taatagtggactcttgttccaaactggaacaacactcaaccctatctcggtctattcttttgatttat
aagggattttgccgatttcggcctattggttaaaaaatgagctgatttaacaaaaatttaacgcgaat
taattctgtggaatgtgtgtcagttagggtgtggaaagtccccaggctccccagcaggcagaagtatg
caaagcatgcatctcaattagtcagcaaccaggtgtggaaagtccccaggctccccagcaggcagaag
tatgcaaagcatgcatctcaattagtcagcaaccatagtcccgcccctaactccgcccatcccgcccc
taactccgcccagttccgcccattctccgccccatggctgactaattttttttatttatgcagaggcc
gaggccgcctctgcctctgagctattccagaagtagtgaggaggcttttttggaggcctaggcttttg
caaaaagctcccgggagcttgtatatccattttcggatctgatcaagagacaggatgaggatcgtttc
gcatgattgaacaagatggattgcacgcaggttctccggccgcttgggtggagaggctattcggctat
gactgggcacaacagacaatcggctgctctgatgccgccgtgttccggctgtcagcgcaggggcgccc
ggttctttttgtcaagaccgacctgtccggtgccctgaatgaactgcaggacgaggcagcgcggctat
cgtggctggccacgacgggcgttccttgcgcagctgtgctcgacgttgtcactgaagcgggaagggac
tggctgctattgggcgaagtgccggggcaggatctcctgtcatctcaccttgctcctgccgagaaagt
atccatcatggctgatgcaatgcggcggctgcatacgcttgatccggctacctgcccattcgaccacc
aagcgaaacatcgcatcgagcgagcacgtactcggatggaagccggtcttgtcgatcaggatgatctg
gacgaagagcatcaggggctcgcgccagccgaactgttcgccaggctcaaggcgcgcatgcccgacgg
cgaggatctcgtcgtgacccatggcgatgcctgcttgccgaatatcatggtggaaaatggccgctttt
ctggattcatcgactgtggccggctgggtgtggcggaccgctatcaggacatagcgttggctacccgt
gatattgctgaagagcttggcggcgaatgggctgaccgcttcctcgtgctttacggtatcgccgctcc
cgattcgcagcgcatcgccttctatcgccttcttgacgagttcttctgagcgggactctggggttcga
aatgaccgaccaagcgacgcccaacctgccatcacgagatttcgattccaccgccgccttctatgaaa
ggttgggcttcggaatcgttttccgggacgccggctggatgatcctccagcgcggggatctcatgctg
gagttcttcgcccaccccaacttgtttattgcagcttataatggttacaaataaagcaatagcatcac
aaatttcacaaataaagcatttttttcactgcattctagttgtggtttgtccaaactcatcaatgtat
cttatcatgtctgtataccgtcgacctctagctagagcttggcgtaatcatggtcatagctgtttcct
gtgtgaaattgttatccgctcacaattccacacaacatacgagccggaagcataaagtgtaaagcctg
gggtgcctaatgagtgagctaactcacattaattgcgttgcgctcactgcccgctttccagtcgggaa
acctgtcgtgccagctgcattaatgaatcggccaacgcgcggggagaggcggtttgcgtattgggcgc
tcttccgcttcctcgctcactgactcgctgcgctcggtcgttcggctgcggcgagcggtatcagctca
ctcaaaggcggtaatacggttatccacagaatcaggggataacgcaggaaagaacatgtgagcaaaag
gccagcaaaaggccaggaaccgtaaaaaggccgcgttgctggcgtttttccataggctccgcccccct
gacgagcatcacaaaaatcgacgctcaagtcagaggtggcgaaacccgacaggactataaagatacca
ggcgtttccccctggaagctccctcgtgcgctctcctgttccgaccctgccgcttaccggatacctgt
ccgcctttctcccttcgggaagcgtggcgctttctcatagctcacgctgtaggtatctcagttcggtg
taggtcgttcgctccaagctgggctgtgtgcacgaaccccccgttcagcccgaccgctgcgccttatc
cggtaactatcgtcttgagtccaacccggtaagacacgacttatcgccactggcagcagccactggta
acaggattagcagagcgaggtatgtaggcggtgctacagagttcttgaagtggtggcctaactacggc
tacactagaagaacagtatttggtatctgcgctctgctgaagccagttaccttcggaaaaagagttgg
tagctcttgatccggcaaacaaaccaccgctggtagcggtttttttgtttgcaagcagcagattacgc
gcagaaaaaaaggatctcaagaagatcctttgatcttttctacggggtctgacgctcagtggaacgaa
aactcacgttaagggattttggtcatgagattatcaaaaaggatcttcacctagatccttttaaatta
aaaatgaagttttaaatcaatctaaagtatatatgagtaaacttggtctgacagttaccaatgcttaa
tcagtgaggcacctatctcagcgatctgtctatttcgttcatccatagttgcctgactccccgtcgtg
tagataactacgatacgggagggcttaccatctggccccagtgctgcaatgataccgcgagacccacg
ctcaccggctccagatttatcagcaataaaccagccagccggaagggccgagcgcagaagtggtcctg
caactttatccgcctccatccagtctattaattgttgccgggaagctagagtaagtagttcgccagtt
aatagtttgcgcaacgttgttgccattgctacaggcatcgtggtgtcacgctcgtcgtttggtatggc
ttcattcagctccggttcccaacgatcaaggcgagttacatgatcccccatgttgtgcaaaaaagcgg
ttagctccttcggtcctccgatcgttgtcagaagtaagttggccgcagtgttatcactcatggttatg
gcagcactgcataattctcttactgtcatgccatccgtaagatgcttttctgtgactggtgagtactc
aaccaagtcattctgagaatagtgtatgcggcgaccgagttgctcttgcccggcgtcaatacgggata
ataccgcgccacatagcagaactttaaaagtgctcatcattggaaaacgttcttcggggcgaaaactc
tcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaa
taagggcgacacggaaatgttgaatactcatactcttcctttttcaatattattgaagcatttatcag
ggttattgtctcatgagcggatacatatttgaatgtatttagaaaaataaacaaataggggttccgcg
cacatttccccgaaaagtgccacctgacgtc
pMD123_pcDNA3_SARS-CoV-2_spike_mut_v3
SEQ ID NO: 100
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCAAGATTGCTGACTACAACTACAAACTGCCTGAT
GACTTCACAGGCTGTGTGATTGCCTGGAACAGCAACAACCTGGACAGCAAGGTGGGAGGCAACTACAA
CTACCTCTACAGACTGTTCAGGAAGAGCAACCTGAAACCATTTGAGAGGGACATCAGCACAGAGATTT
ACgctGCTGGCAGCACACCATGTAATGGAGTGGAGGGCgccgctTGTTACTTTCCACTCCAATCCTAT
GGCTTCCAACCAACCAATGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTAAGTTTGATGAGGATGACTCTGAACCTGTGCTGAA
AGGAGTGAAACTGCACTACACCTGAgtttaaacccgctgatcagcctcgactgtgccttctagttgcc
agccatctgttgtttgcccctcccccgtgccttccttgaccctggaaggtgccactcccactgtcctt
tcctaataaaatgaggaaattgcatcgcattgtctgagtaggtgtcattctattctggggggtggggt
ggggcaggacagcaagggggaggattgggaagacaatagcaggcatgctggggatgcggtgggctcta
tggcttctgaggcggaaagaaccagctggggctctagggggtatccccacgcgccctgtagcggcgca
ttaagcgcggcgggtgtggtggttacgcgcagcgtgaccgctacacttgccagcgccctagcgcccgc
tcctttcgctttcttcccttcctttctcgccacgttcgccggctttccccgtcaagctctaaatcggg
ggctccctttagggttccgatttagtgctttacggcacctcgaccccaaaaaacttgattagggtgat
ggttcacgtagtgggccatcgccctgatagacggtttttcgccctttgacgttggagtccacgttctt
taatagtggactcttgttccaaactggaacaacactcaaccctatctcggtctattcttttgatttat
aagggattttgccgatttcggcctattggttaaaaaatgagctgatttaacaaaaatttaacgcgaat
taattctgtggaatgtgtgtcagttagggtgtggaaagtccccaggctccccagcaggcagaagtatg
caaagcatgcatctcaattagtcagcaaccaggtgtggaaagtccccaggctccccagcaggcagaag
tatgcaaagcatgcatctcaattagtcagcaaccatagtcccgcccctaactccgcccatcccgcccc
taactccgcccagttccgcccattctccgccccatggctgactaattttttttatttatgcagaggcc
gaggccgcctctgcctctgagctattccagaagtagtgaggaggcttttttggaggcctaggcttttg
caaaaagctcccgggagcttgtatatccattttcggatctgatcaagagacaggatgaggatcgtttc
gcatgattgaacaagatggattgcacgcaggttctccggccgcttgggtggagaggctattcggctat
gactgggcacaacagacaatcggctgctctgatgccgccgtgttccggctgtcagcgcaggggcgccc
ggttctttttgtcaagaccgacctgtccggtgccctgaatgaactgcaggacgaggcagcgcggctat
cgtggctggccacgacgggcgttccttgcgcagctgtgctcgacgttgtcactgaagcgggaagggac
tggctgctattgggcgaagtgccggggcaggatctcctgtcatctcaccttgctcctgccgagaaagt
atccatcatggctgatgcaatgcggcggctgcatacgcttgatccggctacctgcccattcgaccacc
aagcgaaacatcgcatcgagcgagcacgtactcggatggaagccggtcttgtcgatcaggatgatctg
gacgaagagcatcaggggctcgcgccagccgaactgttcgccaggctcaaggcgcgcatgcccgacgg
cgaggatctcgtcgtgacccatggcgatgcctgcttgccgaatatcatggtggaaaatggccgctttt
ctggattcatcgactgtggccggctgggtgtggcggaccgctatcaggacatagcgttggctacccgt
gatattgctgaagagcttggcggcgaatgggctgaccgcttcctcgtgctttacggtatcgccgctcc
cgattcgcagcgcatcgccttctatcgccttcttgacgagttcttctgagcgggactctggggttcga
aatgaccgaccaagcgacgcccaacctgccatcacgagatttcgattccaccgccgccttctatgaaa
ggttgggcttcggaatcgttttccgggacgccggctggatgatcctccagcgcggggatctcatgctg
gagttcttcgcccaccccaacttgtttattgcagcttataatggttacaaataaagcaatagcatcac
aaatttcacaaataaagcatttttttcactgcattctagttgtggtttgtccaaactcatcaatgtat
cttatcatgtctgtataccgtcgacctctagctagagcttggcgtaatcatggtcatagctgtttcct
gtgtgaaattgttatccgctcacaattccacacaacatacgagccggaagcataaagtgtaaagcctg
gggtgcctaatgagtgagctaactcacattaattgcgttgcgctcactgcccgctttccagtcgggaa
acctgtcgtgccagctgcattaatgaatcggccaacgcgcggggagaggcggtttgcgtattgggcgc
tcttccgcttcctcgctcactgactcgctgcgctcggtcgttcggctgcggcgagcggtatcagctca
ctcaaaggcggtaatacggttatccacagaatcaggggataacgcaggaaagaacatgtgagcaaaag
gccagcaaaaggccaggaaccgtaaaaaggccgcgttgctggcgtttttccataggctccgcccccct
gacgagcatcacaaaaatcgacgctcaagtcagaggtggcgaaacccgacaggactataaagatacca
ggcgtttccccctggaagctccctcgtgcgctctcctgttccgaccctgccgcttaccggatacctgt
ccgcctttctcccttcgggaagcgtggcgctttctcatagctcacgctgtaggtatctcagttcggtg
taggtcgttcgctccaagctgggctgtgtgcacgaaccccccgttcagcccgaccgctgcgccttatc
cggtaactatcgtcttgagtccaacccggtaagacacgacttatcgccactggcagcagccactggta
acaggattagcagagcgaggtatgtaggcggtgctacagagttcttgaagtggtggcctaactacggc
tacactagaagaacagtatttggtatctgcgctctgctgaagccagttaccttcggaaaaagagttgg
tagctcttgatccggcaaacaaaccaccgctggtagcggttttttttttgcaagcagcagattacgc
gcagaaaaaaaggatctcaagaagatcctttgatcttttctacggggtctgacgctcagtggaacgaa
aactcacgttaagggattttggtcatgagattatcaaaaaggatcttcacctagatccttttaaatta
aaaatgaagttttaaatcaatctaaagtatatatgagtaaacttggtctgacagttaccaatgcttaa
tcagtgaggcacctatctcagcgatctgtctatttcgttcatccatagttgcctgactccccgtcgtg
tagataactacgatacgggagggcttaccatctggccccagtgctgcaatgataccgcgagacccacg
ctcaccggctccagatttatcagcaataaaccagccagccggaagggccgagcgcagaagtggtcctg
caactttatccgcctccatccagtctattaattgttgccgggaagctagagtaagtagttcgccagtt
aatagtttgcgcaacgttgttgccattgctacaggcatcgtggtgtcacgctcgtcgtttggtatggc
ttcattcagctccggttcccaacgatcaaggcgagttacatgatcccccatgttgtgcaaaaaagcgg
ttagctccttcggtcctccgatcgttgtcagaagtaagttggccgcagtgttatcactcatggttatg
gcagcactgcataattctcttactgtcatgccatccgtaagatgcttttctgtgactggtgagtactc
aaccaagtcattctgagaatagtgtatgcggcgaccgagttgctcttgcccggcgtcaatacgggata
ataccgcgccacatagcagaactttaaaagtgctcatcattggaaaacgttcttcggggcgaaaactc
tcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaa
taagggcgacacggaaatgttgaatactcatactcttcctttttcaatattattgaagcatttatcag
ggttattgtctcatgagcggatacatatttgaatgtatttagaaaaataaacaaataggggttccgcg
cacatttccccgaaaagtgccacctgacgtc
pMD124_pcDNA3_SARS-CoV-2_spike_mut_v4
SEQ ID NO: 101
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCgccATTGCTGACTACAACTACAAACTGCCTGAT
GACTTCACAGGCTGTGTGATTGCCTGGAACAGCgccAACCTGGACAGCAAGGTGGGAGGCAACgccAA
CTACCTCTACAGAgccgctAGGAAGAGCAACCTGAAACCATTTGAGAGGGACATCAGCACAGAGATTg
ccgctGCTGGCAGCACACCATGTAATGGAGTGGAGGGCgccgctTGTTACTTTCCACTCgccTCCTAT
GGCTTCgccCCAgctgccGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACgccAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTAAGTTTGATGAGGATGACTCTGAACCTGTGCTGAA
AGGAGTGAAACTGCACTACACCTGAgtttaaacccgctgatcagcctcgactgtgccttctagttgcc
agccatctgttgtttgcccctcccccgtgccttccttgaccctggaaggtgccactcccactgtcctt
tcctaataaaatgaggaaattgcatcgcattgtctgagtaggtgtcattctattctggggggtggggt
ggggcaggacagcaagggggaggattgggaagacaatagcaggcatgctggggatgcggtgggctcta
tggcttctgaggcggaaagaaccagctggggctctagggggtatccccacgcgccctgtagcggcgca
ttaagcgcggcgggtgtggtggttacgcgcagcgtgaccgctacacttgccagcgccctagcgcccgc
tcctttcgctttcttcccttcctttctcgccacgttcgccggctttccccgtcaagctctaaatcggg
ggctccctttagggttccgatttagtgctttacggcacctcgaccccaaaaaacttgattagggtgat
ggttcacgtagtgggccatcgccctgatagacggtttttcgccctttgacgttggagtccacgttctt
taatagtggactcttgttccaaactggaacaacactcaaccctatctcggtctattcttttgatttat
aagggattttgccgatttcggcctattggttaaaaaatgagctgatttaacaaaaatttaacgcgaat
taattctgtggaatgtgtgtcagttagggtgtggaaagtccccaggctccccagcaggcagaagtatg
caaagcatgcatctcaattagtcagcaaccaggtgtggaaagtccccaggctccccagcaggcagaag
tatgcaaagcatgcatctcaattagtcagcaaccatagtcccgcccctaactccgcccatcccgcccc
taactccgcccagttccgcccattctccgccccatggctgactaattttttttatttatgcagaggcc
gaggccgcctctgcctctgagctattccagaagtagtgaggaggcttttttggaggcctaggcttttg
caaaaagctcccgggagcttgtatatccattttcggatctgatcaagagacaggatgaggatcgtttc
gcatgattgaacaagatggattgcacgcaggttctccggccgcttgggtggagaggctattcggctat
gactgggcacaacagacaatcggctgctctgatgccgccgtgttccggctgtcagcgcaggggcgccc
ggttctttttgtcaagaccgacctgtccggtgccctgaatgaactgcaggacgaggcagcgcggctat
cgtggctggccacgacgggcgttccttgcgcagctgtgctcgacgttgtcactgaagcgggaagggac
tggctgctattgggcgaagtgccggggcaggatctcctgtcatctcaccttgctcctgccgagaaagt
atccatcatggctgatgcaatgcggcggctgcatacgcttgatccggctacctgcccattcgaccacc
aagcgaaacatcgcatcgagcgagcacgtactcggatggaagccggtcttgtcgatcaggatgatctg
gacgaagagcatcaggggctcgcgccagccgaactgttcgccaggctcaaggcgcgcatgcccgacgg
cgaggatctcgtcgtgacccatggcgatgcctgcttgccgaatatcatggtggaaaatggccgctttt
ctggattcatcgactgtggccggctgggtgtggcggaccgctatcaggacatagcgttggctacccgt
gatattgctgaagagcttggcggcgaatgggctgaccgcttcctcgtgctttacggtatcgccgctcc
cgattcgcagcgcatcgccttctatcgccttcttgacgagttcttctgagcgggactctggggttcga
aatgaccgaccaagcgacgcccaacctgccatcacgagatttcgattccaccgccgccttctatgaaa
ggttgggcttcggaatcgttttccgggacgccggctggatgatcctccagcgcggggatctcatgctg
gagttcttcgcccaccccaacttgtttattgcagcttataatggttacaaataaagcaatagcatcac
aaatttcacaaataaagcatttttttcactgcattctagttgtggtttgtccaaactcatcaatgtat
cttatcatgtctgtataccgtcgacctctagctagagcttggcgtaatcatggtcatagctgtttcct
gtgtgaaattgttatccgctcacaattccacacaacatacgagccggaagcataaagtgtaaagcctg
gggtgcctaatgagtgagctaactcacattaattgcgttgcgctcactgcccgctttccagtcgggaa
acctgtcgtgccagctgcattaatgaatcggccaacgcgcggggagaggcggtttgcgtattgggcgc
tcttccgcttcctcgctcactgactcgctgcgctcggtcgttcggctgcggcgagcggtatcagctca
ctcaaaggcggtaatacggttatccacagaatcaggggataacgcaggaaagaacatgtgagcaaaag
gccagcaaaaggccaggaaccgtaaaaaggccgcgttgctggcgtttttccataggctccgcccccct
gacgagcatcacaaaaatcgacgctcaagtcagaggtggcgaaacccgacaggactataaagatacca
ggcgtttccccctggaagctccctcgtgcgctctcctgttccgaccctgccgcttaccggatacctgt
ccgcctttctcccttcgggaagcgtggcgctttctcatagctcacgctgtaggtatctcagttcggtg
taggtcgttcgctccaagctgggctgtgtgcacgaaccccccgttcagcccgaccgctgcgccttatc
cggtaactatcgtcttgagtccaacccggtaagacacgacttatcgccactggcagcagccactggta
acaggattagcagagcgaggtatgtaggcggtgctacagagttcttgaagtggtggcctaactacggc
tacactagaagaacagtatttggtatctgcgctctgctgaagccagttaccttcggaaaaagagttgg
tagctcttgatccggcaaacaaaccaccgctggtagcggtttttttgtttgcaagcagcagattacgc
gcagaaaaaaaggatctcaagaagatcctttgatcttttctacggggtctgacgctcagtggaacgaa
aactcacgttaagggattttggtcatgagattatcaaaaaggatcttcacctagatccttttaaatta
aaaatgaagttttaaatcaatctaaagtatatatgagtaaacttggtctgacagttaccaatgcttaa
tcagtgaggcacctatctcagcgatctgtctatttcgttcatccatagttgcctgactccccgtcgtg
tagataactacgatacgggagggcttaccatctggccccagtgctgcaatgataccgcgagacccacg
ctcaccggctccagatttatcagcaataaaccagccagccggaagggccgagcgcagaagtggtcctg
caactttatccgcctccatccagtctattaattgttgccgggaagctagagtaagtagttcgccagtt
aatagtttgcgcaacgttgttgccattgctacaggcatcgtggtgtcacgctcgtcgtttggtatggc
ttcattcagctccggttcccaacgatcaaggcgagttacatgatcccccatgttgtgcaaaaaagcgg
ttagctccttcggtcctccgatcgttgtcagaagtaagttggccgcagtgttatcactcatggttatg
gcagcactgcataattctcttactgtcatgccatccgtaagatgcttttctgtgactggtgagtactc
aaccaagtcattctgagaatagtgtatgcggcgaccgagttgctcttgcccggcgtcaatacgggata
ataccgcgccacatagcagaactttaaaagtgctcatcattggaaaacgttcttcggggcgaaaactc
tcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaa
taagggcgacacggaaatgttgaatactcatactcttcctttttcaatattattgaagcatttatcag
ggttattgtctcatgagcggatacatatttgaatgtatttagaaaaataaacaaataggggttccgcg
cacatttccccgaaaagtgccacctgacgtc
pMD125-pcDNA3.1-SARS2-GGSG-Myc-T2A-BFP-3xFLAG
SEQ ID NO: 102
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCAAGATTGCTGACTACAACTACAAACTGCCTGAT
GACTTCACAGGCTGTGTGATTGCCTGGAACAGCAACAACCTGGACAGCAAGGTGGGAGGCAACTACAA
CTACCTCTACAGACTGTTCAGGAAGAGCAACCTGAAACCATTTGAGAGGGACATCAGCACAGAGATTT
ACCAGGCTGGCAGCACACCATGTAATGGAGTGGAGGGCTTCAACTGTTACTTTCCACTCCAATCCTAT
GGCTTCCAACCAACCAATGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTAAGTTTGATGAGGATGACTCTGAACCTGTGCTGAA
AGGAGTGAAACTGCACTACACCggcggaggcagcggggagcaaaaacttatctctgaagaggacctcg
gcagtggagagggcagaggaagtctgctaacatgcggtgacgtcgaggagaatcctggcccaatggtg
tctaagggcgaagagctgattaaggagaacatgcacatgaagctgtacatggagggcaccgtggacaa
ccatcacttcaagtgcacatccgagggcgaaggcaagccctacgagggcacccagaccatgagaatca
aggtggtcgagggcggccctctccccttcgccttcgacatcctggctactagcttcctctacggcagc
aagaccttcatcaaccacacccagggcatccccgacttcttcaagcagtccttccctgagggcttcac
atgggagagagtcaccacatacgaagatgggggcgtgctgaccgctacccaggacaccagcctccagg
acggctgcctcatctacaacgtcaagatcagaggggtgaacttcacatccaacggccctgtgatgcag
aagaaaacactcggctgggaggccttcaccgagacgctgtaccccgctgacggcggcctggaaggcag
aaacgacatggccctgaagctcgtgggcgggagccatctgatcgcaaacgccaagaccacatatagat
ccaagaaacccgctaagaacctcaagatgcctggcgtctactatgtggactacagactggaaagaatc
aaggaggccaacaacgagacctacgtcgagcagcacgaggtggcagtggccagatactgcgacctccc
tagcaaactggggcacaagcttaatacgcgtgactacaaagaccatgacggtgattataaagatcatg
acatcgattacaaggatgacgatgacaagtaagtttaaacccgctgatcagcctcgactgtgccttct
agttgccagccatctgttgtttgcccctcccccgtgccttccttgaccctggaaggtgccactcccac
tgtcctttcctaataaaatgaggaaattgcatcgcattgtctgagtaggtgtcattctattctggggg
gtggggtggggcaggacagcaagggggaggattgggaagacaatagcaggcatgctggggatgcggtg
ggctctatggcttctgaggcggaaagaaccagctggggctctagggggtatccccacgcgccctgtag
cggcgcattaagcgcggcgggtgtggtggttacgcgcagcgtgaccgctacacttgccagcgccctag
cgcccgctcctttcgctttcttcccttcctttctcgccacgttcgccggctttccccgtcaagctcta
aatcgggggctccctttagggttccgatttagtgctttacggcacctcgaccccaaaaaacttgatta
gggtgatggttcacgtagtgggccatcgccctgatagacggtttttcgccctttgacgttggagtcca
cgttctttaatagtggactcttgttccaaactggaacaacactcaaccctatctcggtctattctttt
gatttataagggattttgccgatttcggcctattggttaaaaaatgagctgatttaacaaaaatttaa
cgcgaattaattctgtggaatgtgtgtcagttagggtgtggaaagtccccaggctccccagcaggcag
aagtatgcaaagcatgcatctcaattagtcagcaaccaggtgtggaaagtccccaggctccccagcag
gcagaagtatgcaaagcatgcatctcaattagtcagcaaccatagtcccgcccctaactccgcccatc
ccgcccctaactccgcccagttccgcccattctccgccccatggctgactaattttttttatttatgc
agaggccgaggccgcctctgcctctgagctattccagaagtagtgaggaggcttttttggaggcctag
gcttttgcaaaaagctcccgggagcttgtatatccattttcggatctgatcaagagacaggatgagga
tcgtttcgcatgattgaacaagatggattgcacgcaggttctccggccgcttgggtggagaggctatt
cggctatgactgggcacaacagacaatcggctgctctgatgccgccgtgttccggctgtcagcgcagg
ggcgcccggttctttttgtcaagaccgacctgtccggtgccctgaatgaactgcaggacgaggcagcg
cggctatcgtggctggccacgacgggcgttccttgcgcagctgtgctcgacgttgtcactgaagcggg
aagggactggctgctattgggcgaagtgccggggcaggatctcctgtcatctcaccttgctcctgccg
agaaagtatccatcatggctgatgcaatgcggcggctgcatacgcttgatccggctacctgcccattc
gaccaccaagcgaaacatcgcatcgagcgagcacgtactcggatggaagccggtcttgtcgatcagga
tgatctggacgaagagcatcaggggctcgcgccagccgaactgttcgccaggctcaaggcgcgcatgc
ccgacggcgaggatctcgtcgtgacccatggcgatgcctgcttgccgaatatcatggtggaaaatggc
cgcttttctggattcatcgactgtggccggctgggtgtggcggaccgctatcaggacatagcgttggc
tacccgtgatattgctgaagagcttggcggcgaatgggctgaccgcttcctcgtgctttacggtatcg
ccgctcccgattcgcagcgcatcgccttctatcgccttcttgacgagttcttctgagcgggactctgg
ggttcgaaatgaccgaccaagcgacgcccaacctgccatcacgagatttcgattccaccgccgccttc
tatgaaaggttgggcttcggaatcgttttccgggacgccggctggatgatcctccagcgcggggatct
catgctggagttcttcgcccaccccaacttgtttattgcagcttataatggttacaaataaagcaata
gcatcacaaatttcacaaataaagcatttttttcactgcattctagttgtggtttgtccaaactcatc
aatgtatcttatcatgtctgtataccgtcgacctctagctagagcttggcgtaatcatggtcatagct
gtttcctgtgtgaaattgttatccgctcacaattccacacaacatacgagccggaagcataaagtgta
aagcctggggtgcctaatgagtgagctaactcacattaattgcgttgcgctcactgcccgctttccag
tcgggaaacctgtcgtgccagctgcattaatgaatcggccaacgcgcggggagaggcggtttgcgtat
tgggcgctcttccgcttcctcgctcactgactcgctgcgctcggtcgttcggctgcggcgagcggtat
cagctcactcaaaggcggtaatacggttatccacagaatcaggggataacgcaggaaagaacatgtga
gcaaaaggccagcaaaaggccaggaaccgtaaaaaggccgcgttgctggcgtttttccataggctccg
cccccctgacgagcatcacaaaaatcgacgctcaagtcagaggtggcgaaacccgacaggactataaa
gataccaggcgtttccccctggaagctccctcgtgcgctctcctgttccgaccctgccgcttaccgga
tacctgtccgcctttctcccttcgggaagcgtggcgctttctcatagctcacgctgtaggtatctcag
ttcggtgtaggtcgttcgctccaagctgggctgtgtgcacgaaccccccgttcagcccgaccgctgcg
ccttatccggtaactatcgtcttgagtccaacccggtaagacacgacttatcgccactggcagcagcc
actggtaacaggattagcagagcgaggtatgtaggcggtgctacagagttcttgaagtggtggcctaa
ctacggctacactagaagaacagtatttggtatctgcgctctgctgaagccagttaccttcggaaaaa
gagttggtagctcttgatccggcaaacaaaccaccgctggtagcggtttttttgtttgcaagcagcag
attacgcgcagaaaaaaaggatctcaagaagatcctttgatcttttctacggggtctgacgctcagtg
gaacgaaaactcacgttaagggattttggtcatgagattatcaaaaaggatcttcacctagatccttt
taaattaaaaatgaagttttaaatcaatctaaagtatatatgagtaaacttggtctgacagttaccaa
tgcttaatcagtgaggcacctatctcagcgatctgtctatttcgttcatccatagttgcctgactccc
cgtcgtgtagataactacgatacgggagggcttaccatctggccccagtgctgcaatgataccgcgag
acccacgctcaccggctccagatttatcagcaataaaccagccagccggaagggccgagcgcagaagt
ggtcctgcaactttatccgcctccatccagtctattaattgttgccgggaagctagagtaagtagttc
gccagttaatagtttgcgcaacgttgttgccattgctacaggcatcgtggtgtcacgctcgtcgtttg
gtatggcttcattcagctccggttcccaacgatcaaggcgagttacatgatcccccatgttgtgcaaa
aaagcggttagctccttcggtcctccgatcgttgtcagaagtaagttggccgcagtgttatcactcat
ggttatggcagcactgcataattctcttactgtcatgccatccgtaagatgcttttctgtgactggtg
agtactcaaccaagtcattctgagaatagtgtatgcggcgaccgagttgctcttgcccggcgtcaata
cgggataataccgcgccacatagcagaactttaaaagtgctcatcattggaaaacgttcttcggggcg
aaaactctcaaggatcttaccgctgttgagatccagttcgatgtaacccactcgtgcacccaactgat
cttcagcatcttttactttcaccagcgtttctgggtgagcaaaaacaggaaggcaaaatgccgcaaaa
aagggaataagggcgacacggaaatgttgaatactcatactcttcctttttcaatattattgaagcat
ttatcagggttattgtctcatgagcggatacatatttgaatgtatttagaaaaataaacaaatagggg
ttccgcgcacatttccccgaaaagtgccacctgacgtc
pMD126-pcDNA3.1-SARS2-WT-GGSG-Myc-T2A-mScarlet
SEQ ID NO: 103
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCAAGATTGCTGACTACAACTACAAACTGCCTGAT
GACTTCACAGGCTGTGTGATTGCCTGGAACAGCAACAACCTGGACAGCAAGGTGGGAGGCAACTACAA
CTACCTCTACAGACTGTTCAGGAAGAGCAACCTGAAACCATTTGAGAGGGACATCAGCACAGAGATTT
ACCAGGCTGGCAGCACACCATGTAATGGAGTGGAGGGCTTCAACTGTTACTTTCCACTCCAATCCTAT
GGCTTCCAACCAACCAATGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTAAGTTTGATGAGGATGACTCTGAACCTGTGCTGAA
AGGAGTGAAACTGCACTACACCggcggaggcagcggggagcaaaaacttatctctgaagaggacctcg
gcagtggagagggcagaggaagtctgctaacatgcggtgacgtcgaggagaatcctggcccaatgagt
aaaggagaagctgtgattaaagagttcatgcgcttcaaagttcacatggagggttctatgaacggtca
cgagttcgagatcgaaggcgaaggcgagggccgtccgtatgaaggcacccagaccgccaaactgaaag
tgactaaaggcggcccgctgcctttttcctgggacatcctgagcccgcaatttatgtacggttctagg
gcgttcaccaaacacccagcggatatcccggactattataagcagtcttttccggaaggtttcaagtg
ggaacgcgtaatgaattttgaagatggtggtgccgtgaccgtcactcaggacacctccctggaggatg
gcaccctgatctataaagttaaactgcgtggtactaattttccacctgatggcccggtgatgcagaaa
aagacgatgggttgggaggcgtctaccgaacgcttgtatccggaagatggtgtgctgaaaggcgacat
taaaatggccctgcgcctgaaagatggcggccgctatctggctgacttcaaaaccacgtacaaagcca
agaaacctgtgcagatgcctggcgcgtacaatgtggaccgcaaactggacatcacctctcataatgaa
gattatacggtggtagagcaatatgagcgctccgagggtcgtcattctaccggtggcatggatgaact
atacaaataagtttaaacccgctgatcagcctcgactgtgccttctagttgccagccatctgttgttt
gcccctcccccgtgccttccttgaccctggaaggtgccactcccactgtcctttcctaataaaatgag
gaaattgcatcgcattgtctgagtaggtgtcattctattctggggggtggggtggggcaggacagcaa
gggggaggattgggaagacaatagcaggcatgctggggatgcggtgggctctatggcttctgaggcgg
aaagaaccagctggggctctagggggtatccccacgcgccctgtagcggcgcattaagcgcggcgggt
gtggtggttacgcgcagcgtgaccgctacacttgccagcgccctagcgcccgctcctttcgctttctt
cccttcctttctcgccacgttcgccggctttccccgtcaagctctaaatcgggggctccctttagggt
tccgatttagtgctttacggcacctcgaccccaaaaaacttgattagggtgatggttcacgtagtggg
ccatcgccctgatagacggtttttcgccctttgacgttggagtccacgttctttaatagtggactctt
gttccaaactggaacaacactcaaccctatctcggtctattcttttgatttataagggattttgccga
tttcggcctattggttaaaaaatgagctgatttaacaaaaatttaacgcgaattaattctgtggaatg
tgtgtcagttagggtgtggaaagtccccaggctccccagcaggcagaagtatgcaaagcatgcatctc
aattagtcagcaaccaggtgtggaaagtccccaggctccccagcaggcagaagtatgcaaagcatgca
tctcaattagtcagcaaccatagtcccgcccctaactccgcccatcccgcccctaactccgcccagtt
ccgcccattctccgccccatggctgactaattttttttatttatgcagaggccgaggccgcctctgcc
tctgagctattccagaagtagtgaggaggcttttttggaggcctaggcttttgcaaaaagctcccggg
agcttgtatatccattttcggatctgatcaagagacaggatgaggatcgtttcgcatgattgaacaag
atggattgcacgcaggttctccggccgcttgggtggagaggctattcggctatgactgggcacaacag
acaatcggctgctctgatgccgccgtgttccggctgtcagcgcaggggcgcccggttctttttgtcaa
gaccgacctgtccggtgccctgaatgaactgcaggacgaggcagcgcggctatcgtggctggccacga
cgggcgttccttgcgcagctgtgctcgacgttgtcactgaagcgggaagggactggctgctattgggc
gaagtgccggggcaggatctcctgtcatctcaccttgctcctgccgagaaagtatccatcatggctga
tgcaatgcggcggctgcatacgcttgatccggctacctgcccattcgaccaccaagcgaaacatcgca
tcgagcgagcacgtactcggatggaagccggtcttgtcgatcaggatgatctggacgaagagcatcag
gggctcgcgccagccgaactgttcgccaggctcaaggcgcgcatgcccgacggcgaggatctcgtcgt
gacccatggcgatgcctgcttgccgaatatcatggtggaaaatggccgcttttctggattcatcgact
gtggccggctgggtgtggcggaccgctatcaggacatagcgttggctacccgtgatattgctgaagag
cttggcggcgaatgggctgaccgcttcctcgtgctttacggtatcgccgctcccgattcgcagcgcat
cgccttctatcgccttcttgacgagttcttctgagcgggactctggggttcgaaatgaccgaccaagc
gacgcccaacctgccatcacgagatttcgattccaccgccgccttctatgaaaggttgggcttcggaa
tcgttttccgggacgccggctggatgatcctccagcgcggggatctcatgctggagttcttcgcccac
cccaacttgtttattgcagcttataatggttacaaataaagcaatagcatcacaaatttcacaaataa
agcatttttttcactgcattctagttgtggtttgtccaaactcatcaatgtatcttatcatgtctgta
taccgtcgacctctagctagagcttggcgtaatcatggtcatagctgtttcctgtgtgaaattgttat
ccgctcacaattccacacaacatacgagccggaagcataaagtgtaaagcctggggtgcctaatgagt
gagctaactcacattaattgcgttgcgctcactgcccgctttccagtcgggaaacctgtcgtgccagc
tgcattaatgaatcggccaacgcgcggggagaggcggtttgcgtattgggcgctcttccgcttcctcg
ctcactgactcgctgcgctcggtcgttcggctgcggcgagcggtatcagctcactcaaaggcggtaat
acggttatccacagaatcaggggataacgcaggaaagaacatgtgagcaaaaggccagcaaaaggcca
ggaaccgtaaaaaggccgcgttgctggcgtttttccataggctccgcccccctgacgagcatcacaaa
aatcgacgctcaagtcagaggtggcgaaacccgacaggactataaagataccaggcgtttccccctgg
aagctccctcgtgcgctctcctgttccgaccctgccgcttaccggatacctgtccgcctttctccctt
cgggaagcgtggcgctttctcatagctcacgctgtaggtatctcagttcggtgtaggtcgttcgctcc
aagctgggctgtgtgcacgaaccccccgttcagcccgaccgctgcgccttatccggtaactatcgtct
tgagtccaacccggtaagacacgacttatcgccactggcagcagccactggtaacaggattagcagag
cgaggtatgtaggcggtgctacagagttcttgaagtggtggcctaactacggctacactagaagaaca
gtatttggtatctgcgctctgctgaagccagttaccttcggaaaaagagttggtagctcttgatccgg
caaacaaaccaccgctggtagcggtttttttgtttgcaagcagcagattacgcgcagaaaaaaaggat
ctcaagaagatcctttgatcttttctacggggtctgacgctcagtggaacgaaaactcacgttaaggg
attttggtcatgagattatcaaaaaggatcttcacctagatccttttaaattaaaaatgaagttttaa
atcaatctaaagtatatatgagtaaacttggtctgacagttaccaatgcttaatcagtgaggcaccta
tctcagcgatctgtctatttcgttcatccatagttgcctgactccccgtcgtgtagataactacgata
cgggagggcttaccatctggccccagtgctgcaatgataccgcgagacccacgctcaccggctccaga
tttatcagcaataaaccagccagccggaagggccgagcgcagaagtggtcctgcaactttatccgcct
ccatccagtctattaattgttgccgggaagctagagtaagtagttcgccagttaatagtttgcgcaac
gttgttgccattgctacaggcatcgtggtgtcacgctcgtcgtttggtatggcttcattcagctccgg
ttcccaacgatcaaggcgagttacatgatcccccatgttgtgcaaaaaagcggttagctccttcggtc
ctccgatcgttgtcagaagtaagttggccgcagtgttatcactcatggttatggcagcactgcataat
tctcttactgtcatgccatccgtaagatgcttttctgtgactggtgagtactcaaccaagtcattctg
agaatagtgtatgcggcgaccgagttgctcttgcccggcgtcaatacgggataataccgcgccacata
gcagaactttaaaagtgctcatcattggaaaacgttcttcggggcgaaaactctcaaggatcttaccg
ctgttgagatccagttcgatgtaacccactcgtgcacccaactgatcttcagcatcttttactttcac
cagcgtttctgggtgagcaaaaacaggaaggcaaaatgccgcaaaaaagggaataagggcgacacgga
aatgttgaatactcatactcttcctttttcaatattattgaagcatttatcagggttattgtctcatg
agcggatacatatttgaatgtatttagaaaaataaacaaataggggttccgcgcacatttccccgaaa
agtgccacctgacgtc
pMD127-pcDNA3.1-SARS2-mut-v1-GGSG-Myc-T2A-mScarlet
SEQ ID NO: 104
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCAAGATTGCTGACTACAACTACAAACTGCCTGAT
GACTTCACAGGCTGTGTGATTGCCTGGAACAGCgccAACCTGGACAGCAAGGTGGGAGGCAACgccAA
CTACCTCTACAGACTGTTCAGGAAGAGCAACCTGAAACCATTTGAGAGGGACATCAGCACAGAGATTT
ACCAGGCTGGCAGCACACCATGTAATGGAGTGGAGGGCTTCAACTGTTACTTTCCACTCCAATCCTAT
GGCTTCgccCCAgctgccGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTAAGTTTGATGAGGATGACTCTGAACCTGTGCTGAA
AGGAGTGAAACTGCACTACACCggcggaggcagcggggagcaaaaacttatctctgaagaggacctcg
gcagtggagagggcagaggaagtctgctaacatgcggtgacgtcgaggagaatcctggcccaatgagt
aaaggagaagctgtgattaaagagttcatgcgcttcaaagttcacatggagggttctatgaacggtca
cgagttcgagatcgaaggcgaaggcgagggccgtccgtatgaaggcacccagaccgccaaactgaaag
tgactaaaggcggcccgctgcctttttcctgggacatcctgagcccgcaatttatgtacggttctagg
gcgttcaccaaacacccagcggatatcccggactattataagcagtcttttccggaaggtttcaagtg
ggaacgcgtaatgaattttgaagatggtggtgccgtgaccgtcactcaggacacctccctggaggatg
gcaccctgatctataaagttaaactgcgtggtactaattttccacctgatggcccggtgatgcagaaa
aagacgatgggttgggaggcgtctaccgaacgcttgtatccggaagatggtgtgctgaaaggcgacat
taaaatggccctgcgcctgaaagatggcggccgctatctggctgacttcaaaaccacgtacaaagcca
agaaacctgtgcagatgcctggcgcgtacaatgtggaccgcaaactggacatcacctctcataatgaa
gattatacggtggtagagcaatatgagcgctccgagggtcgtcattctaccggtggcatggatgaact
atacaaataagtttaaacccgctgatcagcctcgactgtgccttctagttgccagccatctgttgttt
gcccctcccccgtgccttccttgaccctggaaggtgccactcccactgtcctttcctaataaaatgag
gaaattgcatcgcattgtctgagtaggtgtcattctattctggggggtggggtggggcaggacagcaa
gggggaggattgggaagacaatagcaggcatgctggggatgcggtgggctctatggcttctgaggcgg
aaagaaccagctggggctctagggggtatccccacgcgccctgtagcggcgcattaagcgcggcgggt
gtggtggttacgcgcagcgtgaccgctacacttgccagcgccctagcgcccgctcctttcgctttctt
cccttcctttctcgccacgttcgccggctttccccgtcaagctctaaatcgggggctccctttagggt
tccgatttagtgctttacggcacctcgaccccaaaaaacttgattagggtgatggttcacgtagtggg
ccatcgccctgatagacggtttttcgccctttgacgttggagtccacgttctttaatagtggactctt
gttccaaactggaacaacactcaaccctatctcggtctattcttttgatttataagggattttgccga
tttcggcctattggttaaaaaatgagctgatttaacaaaaatttaacgcgaattaattctgtggaatg
tgtgtcagttagggtgtggaaagtccccaggctccccagcaggcagaagtatgcaaagcatgcatctc
aattagtcagcaaccaggtgtggaaagtccccaggctccccagcaggcagaagtatgcaaagcatgca
tctcaattagtcagcaaccatagtcccgcccctaactccgcccatcccgcccctaactccgcccagtt
ccgcccattctccgccccatggctgactaattttttttatttatgcagaggccgaggccgcctctgcc
tctgagctattccagaagtagtgaggaggcttttttggaggcctaggcttttgcaaaaagctcccggg
agcttgtatatccattttcggatctgatcaagagacaggatgaggatcgtttcgcatgattgaacaag
atggattgcacgcaggttctccggccgcttgggtggagaggctattcggctatgactgggcacaacag
acaatcggctgctctgatgccgccgtgttccggctgtcagcgcaggggcgcccggttctttttgtcaa
gaccgacctgtccggtgccctgaatgaactgcaggacgaggcagcgcggctatcgtggctggccacga
cgggcgttccttgcgcagctgtgctcgacgttgtcactgaagcgggaagggactggctgctattgggc
gaagtgccggggcaggatctcctgtcatctcaccttgctcctgccgagaaagtatccatcatggctga
tgcaatgcggcggctgcatacgcttgatccggctacctgcccattcgaccaccaagcgaaacatcgca
tcgagcgagcacgtactcggatggaagccggtcttgtcgatcaggatgatctggacgaagagcatcag
gggctcgcgccagccgaactgttcgccaggctcaaggcgcgcatgcccgacggcgaggatctcgtcgt
gacccatggcgatgcctgcttgccgaatatcatggtggaaaatggccgcttttctggattcatcgact
gtggccggctgggtgtggcggaccgctatcaggacatagcgttggctacccgtgatattgctgaagag
cttggcggcgaatgggctgaccgcttcctcgtgctttacggtatcgccgctcccgattcgcagcgcat
cgccttctatcgccttcttgacgagttcttctgagcgggactctggggttcgaaatgaccgaccaagc
gacgcccaacctgccatcacgagatttcgattccaccgccgccttctatgaaaggttgggcttcggaa
tcgttttccgggacgccggctggatgatcctccagcgcggggatctcatgctggagttcttcgcccac
cccaacttgtttattgcagcttataatggttacaaataaagcaatagcatcacaaatttcacaaataa
agcatttttttcactgcattctagttgtggtttgtccaaactcatcaatgtatcttatcatgtctgta
taccgtcgacctctagctagagcttggcgtaatcatggtcatagctgtttcctgtgtgaaattgttat
ccgctcacaattccacacaacatacgagccggaagcataaagtgtaaagcctggggtgcctaatgagt
gagctaactcacattaattgcgttgcgctcactgcccgctttccagtcgggaaacctgtcgtgccagc
tgcattaatgaatcggccaacgcgcggggagaggcggtttgcgtattgggcgctcttccgcttcctcg
ctcactgactcgctgcgctcggtcgttcggctgcggcgagcggtatcagctcactcaaaggcggtaat
acggttatccacagaatcaggggataacgcaggaaagaacatgtgagcaaaaggccagcaaaaggcca
ggaaccgtaaaaaggccgcgttgctggcgtttttccataggctccgcccccctgacgagcatcacaaa
aatcgacgctcaagtcagaggtggcgaaacccgacaggactataaagataccaggcgtttccccctgg
aagctccctcgtgcgctctcctgttccgaccctgccgcttaccggatacctgtccgcctttctccctt
cgggaagcgtggcgctttctcatagctcacgctgtaggtatctcagttcggtgtaggtcgttcgctcc
aagctgggctgtgtgcacgaaccccccgttcagcccgaccgctgcgccttatccggtaactatcgtct
tgagtccaacccggtaagacacgacttatcgccactggcagcagccactggtaacaggattagcagag
cgaggtatgtaggcggtgctacagagttcttgaagtggtggcctaactacggctacactagaagaaca
gtatttggtatctgcgctctgctgaagccagttaccttcggaaaaagagttggtagctcttgatccgg
caaacaaaccaccgctggtagcggtttttttgtttgcaagcagcagattacgcgcagaaaaaaaggat
ctcaagaagatcctttgatcttttctacggggtctgacgctcagtggaacgaaaactcacgttaaggg
attttggtcatgagattatcaaaaaggatcttcacctagatccttttaaattaaaaatgaagttttaa
atcaatctaaagtatatatgagtaaacttggtctgacagttaccaatgcttaatcagtgaggcaccta
tctcagcgatctgtctatttcgttcatccatagttgcctgactccccgtcgtgtagataactacgata
cgggagggcttaccatctggccccagtgctgcaatgataccgcgagacccacgctcaccggctccaga
tttatcagcaataaaccagccagccggaagggccgagcgcagaagtggtcctgcaactttatccgcct
ccatccagtctattaattgttgccgggaagctagagtaagtagttcgccagttaatagtttgcgcaac
gttgttgccattgctacaggcatcgtggtgtcacgctcgtcgtttggtatggcttcattcagctccgg
ttcccaacgatcaaggcgagttacatgatcccccatgttgtgcaaaaaagcggttagctccttcggtc
ctccgatcgttgtcagaagtaagttggccgcagtgttatcactcatggttatggcagcactgcataat
tctcttactgtcatgccatccgtaagatgcttttctgtgactggtgagtactcaaccaagtcattctg
agaatagtgtatgcggcgaccgagttgctcttgcccggcgtcaatacgggataataccgcgccacata
gcagaactttaaaagtgctcatcattggaaaacgttcttcggggcgaaaactctcaaggatcttaccg
ctgttgagatccagttcgatgtaacccactcgtgcacccaactgatcttcagcatcttttactttcac
cagcgtttctgggtgagcaaaaacaggaaggcaaaatgccgcaaaaaagggaataagggcgacacgga
aatgttgaatactcatactcttcctttttcaatattattgaagcatttatcagggttattgtctcatg
agcggatacatatttgaatgtatttagaaaaataaacaaataggggttccgcgcacatttccccgaaa
agtgccacctgacgtc
pMD128-pcDNA3.1-SARS2-mut-v2-GGSG-Myc-T2A-mScarlet
SEQ ID NO: 105
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCgccATTGCTGACTACAACTACAAACTGCCTGAT
GACTTCACAGGCTGTGTGATTGCCTGGAACAGCAACAACCTGGACAGCAAGGTGGGAGGCAACTACAA
CTACCTCTACAGAgccgctAGGAAGAGCAACCTGAAACCATTTGAGAGGGACATCAGCACAGAGATTg
ccCAGGCTGGCAGCACACCATGTAATGGAGTGGAGGGCTTCAACTGTTACTTTCCACTCgccTCCTAT
GGCTTCCAACCAACCAATGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACgccAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTAAGTTTGATGAGGATGACTCTGAACCTGTGCTGAA
AGGAGTGAAACTGCACTACACCggcggaggcagcggggagcaaaaacttatctctgaagaggacctcg
gcagtggagagggcagaggaagtctgctaacatgcggtgacgtcgaggagaatcctggcccaatgagt
aaaggagaagctgtgattaaagagttcatgcgcttcaaagttcacatggagggttctatgaacggtca
cgagttcgagatcgaaggcgaaggcgagggccgtccgtatgaaggcacccagaccgccaaactgaaag
tgactaaaggcggcccgctgcctttttcctgggacatcctgagcccgcaatttatgtacggttctagg
gcgttcaccaaacacccagcggatatcccggactattataagcagtcttttccggaaggtttcaagtg
ggaacgcgtaatgaattttgaagatggtggtgccgtgaccgtcactcaggacacctccctggaggatg
gcaccctgatctataaagttaaactgcgtggtactaattttccacctgatggcccggtgatgcagaaa
aagacgatgggttgggaggcgtctaccgaacgcttgtatccggaagatggtgtgctgaaaggcgacat
taaaatggccctgcgcctgaaagatggcggccgctatctggctgacttcaaaaccacgtacaaagcca
agaaacctgtgcagatgcctggcgcgtacaatgtggaccgcaaactggacatcacctctcataatgaa
gattatacggtggtagagcaatatgagcgctccgagggtcgtcattctaccggtggcatggatgaact
atacaaataagtttaaacccgctgatcagcctcgactgtgccttctagttgccagccatctgttgttt
gcccctcccccgtgccttccttgaccctggaaggtgccactcccactgtcctttcctaataaaatgag
gaaattgcatcgcattgtctgagtaggtgtcattctattctggggggtggggtggggcaggacagcaa
gggggaggattgggaagacaatagcaggcatgctggggatgcggtgggctctatggcttctgaggcgg
aaagaaccagctggggctctagggggtatccccacgcgccctgtagcggcgcattaagcgcggcgggt
gtggtggttacgcgcagcgtgaccgctacacttgccagcgccctagcgcccgctcctttcgctttctt
cccttcctttctcgccacgttcgccggctttccccgtcaagctctaaatcgggggctccctttagggt
tccgatttagtgctttacggcacctcgaccccaaaaaacttgattagggtgatggttcacgtagtggg
ccatcgccctgatagacggtttttcgccctttgacgttggagtccacgttctttaatagtggactctt
gttccaaactggaacaacactcaaccctatctcggtctattcttttgatttataagggattttgccga
tttcggcctattggttaaaaaatgagctgatttaacaaaaatttaacgcgaattaattctgtggaatg
tgtgtcagttagggtgtggaaagtccccaggctccccagcaggcagaagtatgcaaagcatgcatctc
aattagtcagcaaccaggtgtggaaagtccccaggctccccagcaggcagaagtatgcaaagcatgca
tctcaattagtcagcaaccatagtcccgcccctaactccgcccatcccgcccctaactccgcccagtt
ccgcccattctccgccccatggctgactaattttttttatttatgcagaggccgaggccgcctctgcc
tctgagctattccagaagtagtgaggaggcttttttggaggcctaggcttttgcaaaaagctcccggg
agcttgtatatccattttcggatctgatcaagagacaggatgaggatcgtttcgcatgattgaacaag
atggattgcacgcaggttctccggccgcttgggtggagaggctattcggctatgactgggcacaacag
acaatcggctgctctgatgccgccgtgttccggctgtcagcgcaggggcgcccggttctttttgtcaa
gaccgacctgtccggtgccctgaatgaactgcaggacgaggcagcgcggctatcgtggctggccacga
cgggcgttccttgcgcagctgtgctcgacgttgtcactgaagcgggaagggactggctgctattgggc
gaagtgccggggcaggatctcctgtcatctcaccttgctcctgccgagaaagtatccatcatggctga
tgcaatgcggcggctgcatacgcttgatccggctacctgcccattcgaccaccaagcgaaacatcgca
tcgagcgagcacgtactcggatggaagccggtcttgtcgatcaggatgatctggacgaagagcatcag
gggctcgcgccagccgaactgttcgccaggctcaaggcgcgcatgcccgacggcgaggatctcgtcgt
gacccatggcgatgcctgcttgccgaatatcatggtggaaaatggccgcttttctggattcatcgact
gtggccggctgggtgtggcggaccgctatcaggacatagcgttggctacccgtgatattgctgaagag
cttggcggcgaatgggctgaccgcttcctcgtgctttacggtatcgccgctcccgattcgcagcgcat
cgccttctatcgccttcttgacgagttcttctgagcgggactctggggttcgaaatgaccgaccaagc
gacgcccaacctgccatcacgagatttcgattccaccgccgccttctatgaaaggttgggcttcggaa
tcgttttccgggacgccggctggatgatcctccagcgcggggatctcatgctggagttcttcgcccac
cccaacttgtttattgcagcttataatggttacaaataaagcaatagcatcacaaatttcacaaataa
agcatttttttcactgcattctagttgtggtttgtccaaactcatcaatgtatcttatcatgtctgta
taccgtcgacctctagctagagcttggcgtaatcatggtcatagctgtttcctgtgtgaaattgttat
ccgctcacaattccacacaacatacgagccggaagcataaagtgtaaagcctggggtgcctaatgagt
gagctaactcacattaattgcgttgcgctcactgcccgctttccagtcgggaaacctgtcgtgccagc
tgcattaatgaatcggccaacgcgcggggagaggcggtttgcgtattgggcgctcttccgcttcctcg
ctcactgactcgctgcgctcggtcgttcggctgcggcgagcggtatcagctcactcaaaggcggtaat
acggttatccacagaatcaggggataacgcaggaaagaacatgtgagcaaaaggccagcaaaaggcca
ggaaccgtaaaaaggccgcgttgctggcgtttttccataggctccgcccccctgacgagcatcacaaa
aatcgacgctcaagtcagaggtggcgaaacccgacaggactataaagataccaggcgtttccccctgg
aagctccctcgtgcgctctcctgttccgaccctgccgcttaccggatacctgtccgcctttctccctt
cgggaagcgtggcgctttctcatagctcacgctgtaggtatctcagttcggtgtaggtcgttcgctcc
aagctgggctgtgtgcacgaaccccccgttcagcccgaccgctgcgccttatccggtaactatcgtct
tgagtccaacccggtaagacacgacttatcgccactggcagcagccactggtaacaggattagcagag
cgaggtatgtaggcggtgctacagagttcttgaagtggtggcctaactacggctacactagaagaaca
gtatttggtatctgcgctctgctgaagccagttaccttcggaaaaagagttggtagctcttgatccgg
caaacaaaccaccgctggtagcggtttttttgtttgcaagcagcagattacgcgcagaaaaaaaggat
ctcaagaagatcctttgatcttttctacggggtctgacgctcagtggaacgaaaactcacgttaaggg
attttggtcatgagattatcaaaaaggatcttcacctagatccttttaaattaaaaatgaagttttaa
atcaatctaaagtatatatgagtaaacttggtctgacagttaccaatgcttaatcagtgaggcaccta
tctcagcgatctgtctatttcgttcatccatagttgcctgactccccgtcgtgtagataactacgata
cgggagggcttaccatctggccccagtgctgcaatgataccgcgagacccacgctcaccggctccaga
tttatcagcaataaaccagccagccggaagggccgagcgcagaagtggtcctgcaactttatccgcct
ccatccagtctattaattgttgccgggaagctagagtaagtagttcgccagttaatagtttgcgcaac
gttgttgccattgctacaggcatcgtggtgtcacgctcgtcgtttggtatggcttcattcagctccgg
ttcccaacgatcaaggcgagttacatgatcccccatgttgtgcaaaaaagcggttagctccttcggtc
ctccgatcgttgtcagaagtaagttggccgcagtgttatcactcatggttatggcagcactgcataat
tctcttactgtcatgccatccgtaagatgcttttctgtgactggtgagtactcaaccaagtcattctg
agaatagtgtatgcggcgaccgagttgctcttgcccggcgtcaatacgggataataccgcgccacata
gcagaactttaaaagtgctcatcattggaaaacgttcttcggggcgaaaactctcaaggatcttaccg
ctgttgagatccagttcgatgtaacccactcgtgcacccaactgatcttcagcatcttttactttcac
cagcgtttctgggtgagcaaaaacaggaaggcaaaatgccgcaaaaaagggaataagggcgacacgga
aatgttgaatactcatactcttcctttttcaatattattgaagcatttatcagggttattgtctcatg
agcggatacatatttgaatgtatttagaaaaataaacaaataggggttccgcgcacatttccccgaaa
agtgccacctgacgtc
pMD129-pcDNA3.1-SARS2-mut-v3-GGSG-Myc-T2A-mScarlet
SEQ ID NO: 106
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCAAGATTGCTGACTACAACTACAAACTGCCTGAT
GACTTCACAGGCTGTGTGATTGCCTGGAACAGCAACAACCTGGACAGCAAGGTGGGAGGCAACTACAA
CTACCTCTACAGACTGTTCAGGAAGAGCAACCTGAAACCATTTGAGAGGGACATCAGCACAGAGATTT
ACgctGCTGGCAGCACACCATGTAATGGAGTGGAGGGCgccgctTGTTACTTTCCACTCCAATCCTAT
GGCTTCCAACCAACCAATGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTAAGTTTGATGAGGATGACTCTGAACCTGTGCTGAA
AGGAGTGAAACTGCACTACACCggcggaggcagcggggagcaaaaacttatctctgaagaggacctcg
gcagtggagagggcagaggaagtctgctaacatgcggtgacgtcgaggagaatcctggcccaatgagt
aaaggagaagctgtgattaaagagttcatgcgcttcaaagttcacatggagggttctatgaacggtca
cgagttcgagatcgaaggcgaaggcgagggccgtccgtatgaaggcacccagaccgccaaactgaaag
tgactaaaggcggcccgctgcctttttcctgggacatcctgagcccgcaatttatgtacggttctagg
gcgttcaccaaacacccagcggatatcccggactattataagcagtcttttccggaaggtttcaagtg
ggaacgcgtaatgaattttgaagatggtggtgccgtgaccgtcactcaggacacctccctggaggatg
gcaccctgatctataaagttaaactgcgtggtactaattttccacctgatggcccggtgatgcagaaa
aagacgatgggttgggaggcgtctaccgaacgcttgtatccggaagatggtgtgctgaaaggcgacat
taaaatggccctgcgcctgaaagatggcggccgctatctggctgacttcaaaaccacgtacaaagcca
agaaacctgtgcagatgcctggcgcgtacaatgtggaccgcaaactggacatcacctctcataatgaa
gattatacggtggtagagcaatatgagcgctccgagggtcgtcattctaccggtggcatggatgaact
atacaaataagtttaaacccgctgatcagcctcgactgtgccttctagttgccagccatctgttgttt
gcccctcccccgtgccttccttgaccctggaaggtgccactcccactgtcctttcctaataaaatgag
gaaattgcatcgcattgtctgagtaggtgtcattctattctggggggtggggtggggcaggacagcaa
gggggaggattgggaagacaatagcaggcatgctggggatgcggtgggctctatggcttctgaggcgg
aaagaaccagctggggctctagggggtatccccacgcgccctgtagcggcgcattaagcgcggcgggt
gtggtggttacgcgcagcgtgaccgctacacttgccagcgccctagcgcccgctcctttcgctttctt
cccttcctttctcgccacgttcgccggctttccccgtcaagctctaaatcgggggctccctttagggt
tccgatttagtgctttacggcacctcgaccccaaaaaacttgattagggtgatggttcacgtagtggg
ccatcgccctgatagacggtttttcgccctttgacgttggagtccacgttctttaatagtggactctt
gttccaaactggaacaacactcaaccctatctcggtctattcttttgatttataagggattttgccga
tttcggcctattggttaaaaaatgagctgatttaacaaaaatttaacgcgaattaattctgtggaatg
tgtgtcagttagggtgtggaaagtccccaggctccccagcaggcagaagtatgcaaagcatgcatctc
aattagtcagcaaccaggtgtggaaagtccccaggctccccagcaggcagaagtatgcaaagcatgca
tctcaattagtcagcaaccatagtcccgcccctaactccgcccatcccgcccctaactccgcccagtt
ccgcccattctccgccccatggctgactaattttttttatttatgcagaggccgaggccgcctctgcc
tctgagctattccagaagtagtgaggaggcttttttggaggcctaggcttttgcaaaaagctcccggg
agcttgtatatccattttcggatctgatcaagagacaggatgaggatcgtttcgcatgattgaacaag
atggattgcacgcaggttctccggccgcttgggtggagaggctattcggctatgactgggcacaacag
acaatcggctgctctgatgccgccgtgttccggctgtcagcgcaggggcgcccggttctttttgtcaa
gaccgacctgtccggtgccctgaatgaactgcaggacgaggcagcgcggctatcgtggctggccacga
cgggcgttccttgcgcagctgtgctcgacgttgtcactgaagcgggaagggactggctgctattgggc
gaagtgccggggcaggatctcctgtcatctcaccttgctcctgccgagaaagtatccatcatggctga
tgcaatgcggcggctgcatacgcttgatccggctacctgcccattcgaccaccaagcgaaacatcgca
tcgagcgagcacgtactcggatggaagccggtcttgtcgatcaggatgatctggacgaagagcatcag
gggctcgcgccagccgaactgttcgccaggctcaaggcgcgcatgcccgacggcgaggatctcgtcgt
gacccatggcgatgcctgcttgccgaatatcatggtggaaaatggccgcttttctggattcatcgact
gtggccggctgggtgtggcggaccgctatcaggacatagcgttggctacccgtgatattgctgaagag
cttggcggcgaatgggctgaccgcttcctcgtgctttacggtatcgccgctcccgattcgcagcgcat
cgccttctatcgccttcttgacgagttcttctgagcgggactctggggttcgaaatgaccgaccaagc
gacgcccaacctgccatcacgagatttcgattccaccgccgccttctatgaaaggttgggcttcggaa
tcgttttccgggacgccggctggatgatcctccagcgcggggatctcatgctggagttcttcgcccac
cccaacttgtttattgcagcttataatggttacaaataaagcaatagcatcacaaatttcacaaataa
agcatttttttcactgcattctagttgtggtttgtccaaactcatcaatgtatcttatcatgtctgta
taccgtcgacctctagctagagcttggcgtaatcatggtcatagctgtttcctgtgtgaaattgttat
ccgctcacaattccacacaacatacgagccggaagcataaagtgtaaagcctggggtgcctaatgagt
gagctaactcacattaattgcgttgcgctcactgcccgctttccagtcgggaaacctgtcgtgccagc
tgcattaatgaatcggccaacgcgcggggagaggcggtttgcgtattgggcgctcttccgcttcctcg
ctcactgactcgctgcgctcggtcgttcggctgcggcgagcggtatcagctcactcaaaggcggtaat
acggttatccacagaatcaggggataacgcaggaaagaacatgtgagcaaaaggccagcaaaaggcca
ggaaccgtaaaaaggccgcgttgctggcgtttttccataggctccgcccccctgacgagcatcacaaa
aatcgacgctcaagtcagaggtggcgaaacccgacaggactataaagataccaggcgtttccccctgg
aagctccctcgtgcgctctcctgttccgaccctgccgcttaccggatacctgtccgcctttctccctt
cgggaagcgtggcgctttctcatagctcacgctgtaggtatctcagttcggtgtaggtcgttcgctcc
aagctgggctgtgtgcacgaaccccccgttcagcccgaccgctgcgccttatccggtaactatcgtct
tgagtccaacccggtaagacacgacttatcgccactggcagcagccactggtaacaggattagcagag
cgaggtatgtaggcggtgctacagagttcttgaagtggtggcctaactacggctacactagaagaaca
gtatttggtatctgcgctctgctgaagccagttaccttcggaaaaagagttggtagctcttgatccgg
caaacaaaccaccgctggtagcggtttttttgtttgcaagcagcagattacgcgcagaaaaaaaggat
ctcaagaagatcctttgatcttttctacggggtctgacgctcagtggaacgaaaactcacgttaaggg
attttggtcatgagattatcaaaaaggatcttcacctagatccttttaaattaaaaatgaagttttaa
atcaatctaaagtatatatgagtaaacttggtctgacagttaccaatgcttaatcagtgaggcaccta
tctcagcgatctgtctatttcgttcatccatagttgcctgactccccgtcgtgtagataactacgata
cgggagggcttaccatctggccccagtgctgcaatgataccgcgagacccacgctcaccggctccaga
tttatcagcaataaaccagccagccggaagggccgagcgcagaagtggtcctgcaactttatccgcct
ccatccagtctattaattgttgccgggaagctagagtaagtagttcgccagttaatagtttgcgcaac
gttgttgccattgctacaggcatcgtggtgtcacgctcgtcgtttggtatggcttcattcagctccgg
ttcccaacgatcaaggcgagttacatgatcccccatgttgtgcaaaaaagcggttagctccttcggtc
ctccgatcgttgtcagaagtaagttggccgcagtgttatcactcatggttatggcagcactgcataat
tctcttactgtcatgccatccgtaagatgcttttctgtgactggtgagtactcaaccaagtcattctg
agaatagtgtatgcggcgaccgagttgctcttgcccggcgtcaatacgggataataccgcgccacata
gcagaactttaaaagtgctcatcattggaaaacgttcttcggggcgaaaactctcaaggatcttaccg
ctgttgagatccagttcgatgtaacccactcgtgcacccaactgatcttcagcatcttttactttcac
cagcgtttctgggtgagcaaaaacaggaaggcaaaatgccgcaaaaaagggaataagggcgacacgga
aatgttgaatactcatactcttcctttttcaatattattgaagcatttatcagggttattgtctcatg
agcggatacatatttgaatgtatttagaaaaataaacaaataggggttccgcgcacatttccccgaaa
agtgccacctgacgtc
pMD130-pcDNA3.1-SARS2-mut-v4-GGSG-Myc-T2A-mScarlet
SEQ ID NO: 107
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCgccATTGCTGACTACAACTACAAACTGCCTGAT
GACTTCACAGGCTGTGTGATTGCCTGGAACAGCgccAACCTGGACAGCAAGGTGGGAGGCAACgccAA
CTACCTCTACAGAgccgctAGGAAGAGCAACCTGAAACCATTTGAGAGGGACATCAGCACAGAGATTg
ccgctGCTGGCAGCACACCATGTAATGGAGTGGAGGGCgccgctTGTTACTTTCCACTCgccTCCTAT
GGCTTCgccCCAgctgccGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACgccAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACAAGGTGGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAATATGAACAATACATCAAGTGGCCATGGTACATCTGGCTGGGCTTCA
TTGCTGGACTGATTGCCATTGTGATGGTGACCATAATGCTGTGTTGTATGACCTCCTGTTGTTCCTGT
CTGAAAGGCTGTTGTTCCTGTGGCTCCTGTTGTAAGTTTGATGAGGATGACTCTGAACCTGTGCTGAA
AGGAGTGAAACTGCACTACACCggcggaggcagcggggagcaaaaacttatctctgaagaggacctcg
gcagtggagagggcagaggaagtctgctaacatgcggtgacgtcgaggagaatcctggcccaatgagt
aaaggagaagctgtgattaaagagttcatgcgcttcaaagttcacatggagggttctatgaacggtca
cgagttcgagatcgaaggcgaaggcgagggccgtccgtatgaaggcacccagaccgccaaactgaaag
tgactaaaggcggcccgctgcctttttcctgggacatcctgagcccgcaatttatgtacggttctagg
gcgttcaccaaacacccagcggatatcccggactattataagcagtcttttccggaaggtttcaagtg
ggaacgcgtaatgaattttgaagatggtggtgccgtgaccgtcactcaggacacctccctggaggatg
gcaccctgatctataaagttaaactgcgtggtactaattttccacctgatggcccggtgatgcagaaa
aagacgatgggttgggaggcgtctaccgaacgcttgtatccggaagatggtgtgctgaaaggcgacat
taaaatggccctgcgcctgaaagatggcggccgctatctggctgacttcaaaaccacgtacaaagcca
agaaacctgtgcagatgcctggcgcgtacaatgtggaccgcaaactggacatcacctctcataatgaa
gattatacggtggtagagcaatatgagcgctccgagggtcgtcattctaccggtggcatggatgaact
atacaaataagtttaaacccgctgatcagcctcgactgtgccttctagttgccagccatctgttgttt
gcccctcccccgtgccttccttgaccctggaaggtgccactcccactgtcctttcctaataaaatgag
gaaattgcatcgcattgtctgagtaggtgtcattctattctggggggtggggtggggcaggacagcaa
gggggaggattgggaagacaatagcaggcatgctggggatgcggtgggctctatggcttctgaggcgg
aaagaaccagctggggctctagggggtatccccacgcgccctgtagcggcgcattaagcgcggcgggt
gtggtggttacgcgcagcgtgaccgctacacttgccagcgccctagcgcccgctcctttcgctttctt
cccttcctttctcgccacgttcgccggctttccccgtcaagctctaaatcgggggctccctttagggt
tccgatttagtgctttacggcacctcgaccccaaaaaacttgattagggtgatggttcacgtagtggg
ccatcgccctgatagacggtttttcgccctttgacgttggagtccacgttctttaatagtggactctt
gttccaaactggaacaacactcaaccctatctcggtctattcttttgatttataagggattttgccga
tttcggcctattggttaaaaaatgagctgatttaacaaaaatttaacgcgaattaattctgtggaatg
tgtgtcagttagggtgtggaaagtccccaggctccccagcaggcagaagtatgcaaagcatgcatctc
aattagtcagcaaccaggtgtggaaagtccccaggctccccagcaggcagaagtatgcaaagcatgca
tctcaattagtcagcaaccatagtcccgcccctaactccgcccatcccgcccctaactccgcccagtt
ccgcccattctccgccccatggctgactaattttttttatttatgcagaggccgaggccgcctctgcc
tctgagctattccagaagtagtgaggaggcttttttggaggcctaggcttttgcaaaaagctcccggg
agcttgtatatccattttcggatctgatcaagagacaggatgaggatcgtttcgcatgattgaacaag
atggattgcacgcaggttctccggccgcttgggtggagaggctattcggctatgactgggcacaacag
acaatcggctgctctgatgccgccgtgttccggctgtcagcgcaggggcgcccggttctttttgtcaa
gaccgacctgtccggtgccctgaatgaactgcaggacgaggcagcgcggctatcgtggctggccacga
cgggcgttccttgcgcagctgtgctcgacgttgtcactgaagcgggaagggactggctgctattgggc
gaagtgccggggcaggatctcctgtcatctcaccttgctcctgccgagaaagtatccatcatggctga
tgcaatgcggcggctgcatacgcttgatccggctacctgcccattcgaccaccaagcgaaacatcgca
tcgagcgagcacgtactcggatggaagccggtcttgtcgatcaggatgatctggacgaagagcatcag
gggctcgcgccagccgaactgttcgccaggctcaaggcgcgcatgcccgacggcgaggatctcgtcgt
gacccatggcgatgcctgcttgccgaatatcatggtggaaaatggccgcttttctggattcatcgact
gtggccggctgggtgtggcggaccgctatcaggacatagcgttggctacccgtgatattgctgaagag
cttggcggcgaatgggctgaccgcttcctcgtgctttacggtatcgccgctcccgattcgcagcgcat
cgccttctatcgccttcttgacgagttcttctgagcgggactctggggttcgaaatgaccgaccaagc
gacgcccaacctgccatcacgagatttcgattccaccgccgccttctatgaaaggttgggcttcggaa
tcgttttccgggacgccggctggatgatcctccagcgcggggatctcatgctggagttcttcgcccac
cccaacttgtttattgcagcttataatggttacaaataaagcaatagcatcacaaatttcacaaataa
agcatttttttcactgcattctagttgtggtttgtccaaactcatcaatgtatcttatcatgtctgta
taccgtcgacctctagctagagcttggcgtaatcatggtcatagctgtttcctgtgtgaaattgttat
ccgctcacaattccacacaacatacgagccggaagcataaagtgtaaagcctggggtgcctaatgagt
gagctaactcacattaattgcgttgcgctcactgcccgctttccagtcgggaaacctgtcgtgccagc
tgcattaatgaatcggccaacgcgcggggagaggcggtttgcgtattgggcgctcttccgcttcctcg
ctcactgactcgctgcgctcggtcgttcggctgcggcgagcggtatcagctcactcaaaggcggtaat
acggttatccacagaatcaggggataacgcaggaaagaacatgtgagcaaaaggccagcaaaaggcca
ggaaccgtaaaaaggccgcgttgctggcgtttttccataggctccgcccccctgacgagcatcacaaa
aatcgacgctcaagtcagaggtggcgaaacccgacaggactataaagataccaggcgtttccccctgg
aagctccctcgtgcgctctcctgttccgaccctgccgcttaccggatacctgtccgcctttctccctt
cgggaagcgtggcgctttctcatagctcacgctgtaggtatctcagttcggtgtaggtcgttcgctcc
aagctgggctgtgtgcacgaaccccccgttcagcccgaccgctgcgccttatccggtaactatcgtct
tgagtccaacccggtaagacacgacttatcgccactggcagcagccactggtaacaggattagcagag
cgaggtatgtaggcggtgctacagagttcttgaagtggtggcctaactacggctacactagaagaaca
gtatttggtatctgcgctctgctgaagccagttaccttcggaaaaagagttggtagctcttgatccgg
caaacaaaccaccgctggtagcggtttttttgtttgcaagcagcagattacgcgcagaaaaaaaggat
ctcaagaagatcctttgatcttttctacggggtctgacgctcagtggaacgaaaactcacgttaaggg
attttggtcatgagattatcaaaaaggatcttcacctagatccttttaaattaaaaatgaagttttaa
atcaatctaaagtatatatgagtaaacttggtctgacagttaccaatgcttaatcagtgaggcaccta
tctcagcgatctgtctatttcgttcatccatagttgcctgactccccgtcgtgtagataactacgata
cgggagggcttaccatctggccccagtgctgcaatgataccgcgagacccacgctcaccggctccaga
tttatcagcaataaaccagccagccggaagggccgagcgcagaagtggtcctgcaactttatccgcct
ccatccagtctattaattgttgccgggaagctagagtaagtagttcgccagttaatagtttgcgcaac
gttgttgccattgctacaggcatcgtggtgtcacgctcgtcgtttggtatggcttcattcagctccgg
ttcccaacgatcaaggcgagttacatgatcccccatgttgtgcaaaaaagcggttagctccttcggtc
ctccgatcgttgtcagaagtaagttggccgcagtgttatcactcatggttatggcagcactgcataat
tctcttactgtcatgccatccgtaagatgcttttctgtgactggtgagtactcaaccaagtcattctg
agaatagtgtatgcggcgaccgagttgctcttgcccggcgtcaatacgggataataccgcgccacata
gcagaactttaaaagtgctcatcattggaaaacgttcttcggggcgaaaactctcaaggatcttaccg
ctgttgagatccagttcgatgtaacccactcgtgcacccaactgatcttcagcatcttttactttcac
cagcgtttctgggtgagcaaaaacaggaaggcaaaatgccgcaaaaaagggaataagggcgacacgga
aatgttgaatactcatactcttcctttttcaatattattgaagcatttatcagggttattgtctcatg
agcggatacatatttgaatgtatttagaaaaataaacaaataggggttccgcgcacatttccccgaaa
agtgccacctgacgtc
pMD131_pcDNA3.1_SARS-CoV-2_stabilized-S-protein_mut_v1
SEQ ID NO: 108
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGAGGGCAAG
CAGGGCAACTTCAAGAACCTGAGGGAGTTTGTGTTCAAGAACATTGATGGCTACTTCAAGATTTACAG
CAAACACACACCAATCAACCTGGTGAGGGACCTGCCACAGGGCTTCTCTGCCTTGGAACCACTGGTGG
ACCTGCCAATTGGCATCAACATCACCAGGTTCCAGACCCTGCTGGCTCTGCACAGGTCCTACCTGACA
CCTGGAGACTCCTCCTCTGGCTGGACAGCAGGAGCAGCAGCCTACTATGTGGGCTACCTCCAACCAAG
GACCTTCCTGCTGAAATACAATGAGAATGGCACCATCACAGATGCTGTGGACTGTGCCCTGGACCCAC
TGTCTGAGACCAAGTGTACCCTGAAATCCTTCACAGTGGAGAAGGGCATCTACCAGACCAGCAACTTC
AGGGTCCAACCAACAGAGAGCATTGTGAGGTTTCCAAACATCACCAACCTGTGTCCATTTGGAGAGGT
GTTCAATGCCACCAGGTTTGCCTCTGTCTATGCCTGGAACAGGAAGAGGATTAGCAACTGTGTGGCTG
ACTACTCTGTGCTCTACAACTCTGCCTCCTTCAGCACCTTCAAGTGTTATGGAGTGAGCCCAACCAAA
CTGAATGACCTGTGTTTCACCAATGTCTATGCTGACTCCTTTGTGATTAGGGGAGATGAGGTGAGACA
GATTGCCCCTGGACAAACAGGCAAGATTGCTGACTACAACTACAAACTGCCTGATGACTTCACAGGCT
GTGTGATTGCCTGGAACAGCgccAACCTGGACAGCAAGGTGGGAGGCAACgccAACTACCTCTACAGA
CTGTTCAGGAAGAGCAACCTGAAACCATTTGAGAGGGACATCAGCACAGAGATTTACCAGGCTGGCAG
CACACCATGTAATGGAGTGGAGGGCTTCAACTGTTACTTTCCACTCCAATCCTATGGCTTCgccCCAg
ctgccGGAGTGGGCTACCAACCATACAGGGTGGTGGTGCTGTCCTTTGAACTGCTCCATGCCCCTGCC
ACAGTGTGTGGACCAAAGAAGAGCACCAACCTGGTGAAGAACAAGTGTGTGAACTTCAACTTCAATGG
ACTGACAGGCACAGGAGTGCTGACAGAGAGCAACAAGAAGTTCCTGCCATTCCAACAGTTTGGCAGGG
ACATTGCTGACACCACAGATGCTGTGAGGGACCCACAGACCTTGGAGATTCTGGACATCACACCATGT
TCCTTTGGAGGAGTGTCTGTGATTACACCTGGCACCAACACCAGCAACCAGGTGGCTGTGCTCTACCA
GGATGTGAACTGTACTGAGGTGCCTGTGGCTATCCATGCTGACCAACTTACACCAACCTGGAGGGTCT
ACAGCACAGGCAGCAATGTGTTCCAGACCAGGGCTGGCTGTCTGATTGGAGCAGAGCATGTGAACAAC
TCCTATGAGTGTGACATCCCAATTGGAGCAGGCATCTGTGCCTCCTACCAGACCCAGACCAACAGCCC
AAGGAGGGCAAGGTCTGTGGCAAGCCAGAGCATCATTGCCTACACAATGAGTCTGGGAGCAGAGAACT
CTGTGGCTTACAGCAACAACAGCATTGCCATCCCAACCAACTTCACCATCTCTGTGACCACAGAGATT
CTGCCTGTGAGTATGACCAAGACCTCTGTGGACTGTACAATGTATATCTGTGGAGACAGCACAGAGTG
TAGCAACCTGCTGCTCCAATATGGCTCCTTCTGTACCCAACTTAACAGGGCTCTGACAGGCATTGCTG
TGGAACAGGACAAGAACACCCAGGAGGTGTTTGCCCAGGTGAAGCAGATTTACAAGACACCTCCAATC
AAGGACTTTGGAGGCTTCAACTTCAGCCAGATTCTGCCTGACCCAAGCAAGCCAAGCAAGAGGTCCTT
CATTGAGGACCTGCTGTTCAACAAGGTGACCCTGGCTGATGCTGGCTTCATCAAGCAATATGGAGACT
GTCTGGGAGACATTGCTGCCAGGGACCTGATTTGTGCCCAGAAGTTCAATGGACTGACAGTGCTGCCT
CCACTGCTGACAGATGAGATGATTGCCCAATACACCTCTGCCCTGCTGGCTGGCACCATCACCTCTGG
CTGGACCTTTGGAGCAGGAGCAGCCCTCCAAATCCCATTTGCTATGCAGATGGCTTACAGGTTCAATG
GCATTGGAGTGACCCAGAATGTGCTCTATGAGAACCAGAAACTGATTGCCAACCAGTTCAACTCTGCC
ATTGGCAAGATTCAGGACTCCCTGTCCAGCACAGCCTCTGCCCTGGGCAAACTCCAAGATGTGGTGAA
CCAGAATGCCCAGGCTCTGAACACCCTGGTGAAGCAACTTTCCAGCAACTTTGGAGCCATCTCCTCTG
TGCTGAATGACATCCTGAGCAGACTGGACcctcccGAGGCTGAGGTCCAGATTGACAGACTGATTACA
GGCAGACTCCAATCCCTCCAAACCTATGTGACCCAACAACTTATCAGGGCTGCTGAGATTAGGGCATC
TGCCAACCTGGCTGCCACCAAGATGAGTGAGTGTGTGCTGGGACAAAGCAAGAGGGTGGACTTCTGTG
GCAAGGGCTACCACCTGATGAGTTTTCCACAGTCTGCCCCTCATGGAGTGGTGTTCCTGCATGTGACC
TATGTGCCTGCCCAGGAGAAGAACTTCACCACAGCCCCTGCCATCTGCCATGATGGCAAGGCTCACTT
TCCAAGGGAGGGAGTGTTTGTGAGCAATGGCACCCACTGGTTTGTGACCCAGAGGAACTTCTATGAAC
CACAGATTATCACCACAGACAACACCTTTGTGTCTGGCAACTGTGATGTGGTGATTGGCATTGTGAAC
AACACAGTCTATGACCCACTCCAACCTGAACTGGACTCCTTCAAGGAGGAACTGGACAAATACTTCAA
GAACCACACCAGCCCTGATGTGGACCTGGGAGACATCTCTGGCATCAATGCCTCTGTGGTGAACATCC
AGAAGGAGATTGACAGACTGAATGAGGTGGCTAAGAACCTGAATGAGTCCCTGATTGACCTCCAAGAA
CTGGGCAAATAAgtttaaacccgctgatcagcctcgactgtgccttctagttgccagccatctgttgt
ttgcccctcccccgtgccttccttgaccctggaaggtgccactcccactgtcctttcctaataaaatg
aggaaattgcatcgcattgtctgagtaggtgtcattctattctggggggtggggtggggcaggacagc
aagggggaggattgggaagacaatagcaggcatgctggggatgcggtgggctctatggcttctgaggc
ggaaagaaccagctggggctctagggggtatccccacgcgccctgtagcggcgcattaagcgcggcgg
gtgtggtggttacgcgcagcgtgaccgctacacttgccagcgccctagcgcccgctcctttcgctttc
ttcccttcctttctcgccacgttcgccggctttccccgtcaagctctaaatcgggggctccctttagg
gttccgatttagtgctttacggcacctcgaccccaaaaaacttgattagggtgatggttcacgtagtg
ggccatcgccctgatagacggtttttcgccctttgacgttggagtccacgttctttaatagtggactc
ttgttccaaactggaacaacactcaaccctatctcggtctattcttttgatttataagggattttgcc
gatttcggcctattggttaaaaaatgagctgatttaacaaaaatttaacgcgaattaattctgtggaa
tgtgtgtcagttagggtgtggaaagtccccaggctccccagcaggcagaagtatgcaaagcatgcatc
tcaattagtcagcaaccaggtgtggaaagtccccaggctccccagcaggcagaagtatgcaaagcatg
catctcaattagtcagcaaccatagtcccgcccctaactccgcccatcccgcccctaactccgcccag
ttccgcccattctccgccccatggctgactaattttttttatttatgcagaggccgaggccgcctctg
cctctgagctattccagaagtagtgaggaggcttttttggaggcctaggcttttgcaaaaagctcccg
ggagcttgtatatccattttcggatctgatcaagagacaggatgaggatcgtttcgcatgattgaaca
agatggattgcacgcaggttctccggccgcttgggtggagaggctattcggctatgactgggcacaac
agacaatcggctgctctgatgccgccgtgttccggctgtcagcgcaggggcgcccggttctttttgtc
aagaccgacctgtccggtgccctgaatgaactgcaggacgaggcagcgcggctatcgtggctggccac
gacgggcgttccttgcgcagctgtgctcgacgttgtcactgaagcgggaagggactggctgctattgg
gcgaagtgccggggcaggatctcctgtcatctcaccttgctcctgccgagaaagtatccatcatggct
gatgcaatgcggcggctgcatacgcttgatccggctacctgcccattcgaccaccaagcgaaacatcg
catcgagcgagcacgtactcggatggaagccggtcttgtcgatcaggatgatctggacgaagagcatc
aggggctcgcgccagccgaactgttcgccaggctcaaggcgcgcatgcccgacggcgaggatctcgtc
gtgacccatggcgatgcctgcttgccgaatatcatggtggaaaatggccgcttttctggattcatcga
ctgtggccggctgggtgtggcggaccgctatcaggacatagcgttggctacccgtgatattgctgaag
agcttggcggcgaatgggctgaccgcttcctcgtgctttacggtatcgccgctcccgattcgcagcgc
atcgccttctatcgccttcttgacgagttcttctgagcgggactctggggttcgaaatgaccgaccaa
gcgacgcccaacctgccatcacgagatttcgattccaccgccgccttctatgaaaggttgggcttcgg
aatcgttttccgggacgccggctggatgatcctccagcgcggggatctcatgctggagttcttcgccc
accccaacttgtttattgcagcttataatggttacaaataaagcaatagcatcacaaatttcacaaat
aaagcatttttttcactgcattctagttgtggtttgtccaaactcatcaatgtatcttatcatgtctg
tataccgtcgacctctagctagagcttggcgtaatcatggtcatagctgtttcctgtgtgaaattgtt
atccgctcacaattccacacaacatacgagccggaagcataaagtgtaaagcctggggtgcctaatga
gtgagctaactcacattaattgcgttgcgctcactgcccgctttccagtcgggaaacctgtcgtgcca
gctgcattaatgaatcggccaacgcgcggggagaggcggtttgcgtattgggcgctcttccgcttcct
cgctcactgactcgctgcgctcggtcgttcggctgcggcgagcggtatcagctcactcaaaggcggta
atacggttatccacagaatcaggggataacgcaggaaagaacatgtgagcaaaaggccagcaaaaggc
caggaaccgtaaaaaggccgcgttgctggcgtttttccataggctccgcccccctgacgagcatcaca
aaaatcgacgctcaagtcagaggtggcgaaacccgacaggactataaagataccaggcgtttccccct
ggaagctccctcgtgcgctctcctgttccgaccctgccgcttaccggatacctgtccgcctttctccc
ttcgggaagcgtggcgctttctcatagctcacgctgtaggtatctcagttcggtgtaggtcgttcgct
ccaagctgggctgtgtgcacgaaccccccgttcagcccgaccgctgcgccttatccggtaactatcgt
cttgagtccaacccggtaagacacgacttatcgccactggcagcagccactggtaacaggattagcag
agcgaggtatgtaggcggtgctacagagttcttgaagtggtggcctaactacggctacactagaagaa
cagtatttggtatctgcgctctgctgaagccagttaccttcggaaaaagagttggtagctcttgatcc
ggcaaacaaaccaccgctggtagcggtttttttgtttgcaagcagcagattacgcgcagaaaaaaagg
atctcaagaagatcctttgatcttttctacggggtctgacgctcagtggaacgaaaactcacgttaag
ggattttggtcatgagattatcaaaaaggatcttcacctagatccttttaaattaaaaatgaagtttt
aaatcaatctaaagtatatatgagtaaacttggtctgacagttaccaatgcttaatcagtgaggcacc
tatctcagcgatctgtctatttcgttcatccatagttgcctgactccccgtcgtgtagataactacga
tacgggagggcttaccatctggccccagtgctgcaatgataccgcgagacccacgctcaccggctcca
gatttatcagcaataaaccagccagccggaagggccgagcgcagaagtggtcctgcaactttatccgc
ctccatccagtctattaattgttgccgggaagctagagtaagtagttcgccagttaatagtttgcgca
acgttgttgccattgctacaggcatcgtggtgtcacgctcgtcgtttggtatggcttcattcagctcc
ggttcccaacgatcaaggcgagttacatgatcccccatgttgtgcaaaaaagcggttagctccttcgg
tcctccgatcgttgtcagaagtaagttggccgcagtgttatcactcatggttatggcagcactgcata
attctcttactgtcatgccatccgtaagatgcttttctgtgactggtgagtactcaaccaagtcattc
tgagaatagtgtatgcggcgaccgagttgctcttgcccggcgtcaatacgggataataccgcgccaca
tagcagaactttaaaagtgctcatcattggaaaacgttcttcggggcgaaaactctcaaggatcttac
cgctgttgagatccagttcgatgtaacccactcgtgcacccaactgatcttcagcatcttttactttc
accagcgtttctgggtgagcaaaaacaggaaggcaaaatgccgcaaaaaagggaataagggcgacacg
gaaatgttgaatactcatactcttcctttttcaatattattgaagcatttatcagggttattgtctca
tgagcggatacatatttgaatgtatttagaaaaataaacaaataggggttccgcgcacatttccccga
aaagtgccacctgacgtc
pMD132_pcDNA3.1_SARS-CoV-2_stabilized-S-protein_mut_v2
SEQ ID NO: 109
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGAGGGCAAG
CAGGGCAACTTCAAGAACCTGAGGGAGTTTGTGTTCAAGAACATTGATGGCTACTTCAAGATTTACAG
CAAACACACACCAATCAACCTGGTGAGGGACCTGCCACAGGGCTTCTCTGCCTTGGAACCACTGGTGG
ACCTGCCAATTGGCATCAACATCACCAGGTTCCAGACCCTGCTGGCTCTGCACAGGTCCTACCTGACA
CCTGGAGACTCCTCCTCTGGCTGGACAGCAGGAGCAGCAGCCTACTATGTGGGCTACCTCCAACCAAG
GACCTTCCTGCTGAAATACAATGAGAATGGCACCATCACAGATGCTGTGGACTGTGCCCTGGACCCAC
TGTCTGAGACCAAGTGTACCCTGAAATCCTTCACAGTGGAGAAGGGCATCTACCAGACCAGCAACTTC
AGGGTCCAACCAACAGAGAGCATTGTGAGGTTTCCAAACATCACCAACCTGTGTCCATTTGGAGAGGT
GTTCAATGCCACCAGGTTTGCCTCTGTCTATGCCTGGAACAGGAAGAGGATTAGCAACTGTGTGGCTG
ACTACTCTGTGCTCTACAACTCTGCCTCCTTCAGCACCTTCAAGTGTTATGGAGTGAGCCCAACCAAA
CTGAATGACCTGTGTTTCACCAATGTCTATGCTGACTCCTTTGTGATTAGGGGAGATGAGGTGAGACA
GATTGCCCCTGGACAAACAGGCgccATTGCTGACTACAACTACAAACTGCCTGATGACTTCACAGGCT
GTGTGATTGCCTGGAACAGCAACAACCTGGACAGCAAGGTGGGAGGCAACTACAACTACCTCTACAGA
gccgctAGGAAGAGCAACCTGAAACCATTTGAGAGGGACATCAGCACAGAGATTgccCAGGCTGGCAG
CACACCATGTAATGGAGTGGAGGGCTTCAACTGTTACTTTCCACTCgccTCCTATGGCTTCCAACCAA
CCAATGGAGTGGGCTACCAACCATACAGGGTGGTGGTGCTGTCCTTTGAACTGCTCCATGCCCCTGCC
ACAGTGTGTGGACCAAAGAAGAGCACCAACCTGGTGAAGAACAAGTGTGTGAACTTCAACgccAATGG
ACTGACAGGCACAGGAGTGCTGACAGAGAGCAACAAGAAGTTCCTGCCATTCCAACAGTTTGGCAGGG
ACATTGCTGACACCACAGATGCTGTGAGGGACCCACAGACCTTGGAGATTCTGGACATCACACCATGT
TCCTTTGGAGGAGTGTCTGTGATTACACCTGGCACCAACACCAGCAACCAGGTGGCTGTGCTCTACCA
GGATGTGAACTGTACTGAGGTGCCTGTGGCTATCCATGCTGACCAACTTACACCAACCTGGAGGGTCT
ACAGCACAGGCAGCAATGTGTTCCAGACCAGGGCTGGCTGTCTGATTGGAGCAGAGCATGTGAACAAC
TCCTATGAGTGTGACATCCCAATTGGAGCAGGCATCTGTGCCTCCTACCAGACCCAGACCAACAGCCC
AAGGAGGGCAAGGTCTGTGGCAAGCCAGAGCATCATTGCCTACACAATGAGTCTGGGAGCAGAGAACT
CTGTGGCTTACAGCAACAACAGCATTGCCATCCCAACCAACTTCACCATCTCTGTGACCACAGAGATT
CTGCCTGTGAGTATGACCAAGACCTCTGTGGACTGTACAATGTATATCTGTGGAGACAGCACAGAGTG
TAGCAACCTGCTGCTCCAATATGGCTCCTTCTGTACCCAACTTAACAGGGCTCTGACAGGCATTGCTG
TGGAACAGGACAAGAACACCCAGGAGGTGTTTGCCCAGGTGAAGCAGATTTACAAGACACCTCCAATC
AAGGACTTTGGAGGCTTCAACTTCAGCCAGATTCTGCCTGACCCAAGCAAGCCAAGCAAGAGGTCCTT
CATTGAGGACCTGCTGTTCAACAAGGTGACCCTGGCTGATGCTGGCTTCATCAAGCAATATGGAGACT
GTCTGGGAGACATTGCTGCCAGGGACCTGATTTGTGCCCAGAAGTTCAATGGACTGACAGTGCTGCCT
CCACTGCTGACAGATGAGATGATTGCCCAATACACCTCTGCCCTGCTGGCTGGCACCATCACCTCTGG
CTGGACCTTTGGAGCAGGAGCAGCCCTCCAAATCCCATTTGCTATGCAGATGGCTTACAGGTTCAATG
GCATTGGAGTGACCCAGAATGTGCTCTATGAGAACCAGAAACTGATTGCCAACCAGTTCAACTCTGCC
ATTGGCAAGATTCAGGACTCCCTGTCCAGCACAGCCTCTGCCCTGGGCAAACTCCAAGATGTGGTGAA
CCAGAATGCCCAGGCTCTGAACACCCTGGTGAAGCAACTTTCCAGCAACTTTGGAGCCATCTCCTCTG
TGCTGAATGACATCCTGAGCAGACTGGACcctcccGAGGCTGAGGTCCAGATTGACAGACTGATTACA
GGCAGACTCCAATCCCTCCAAACCTATGTGACCCAACAACTTATCAGGGCTGCTGAGATTAGGGCATC
TGCCAACCTGGCTGCCACCAAGATGAGTGAGTGTGTGCTGGGACAAAGCAAGAGGGTGGACTTCTGTG
GCAAGGGCTACCACCTGATGAGTTTTCCACAGTCTGCCCCTCATGGAGTGGTGTTCCTGCATGTGACC
TATGTGCCTGCCCAGGAGAAGAACTTCACCACAGCCCCTGCCATCTGCCATGATGGCAAGGCTCACTT
TCCAAGGGAGGGAGTGTTTGTGAGCAATGGCACCCACTGGTTTGTGACCCAGAGGAACTTCTATGAAC
CACAGATTATCACCACAGACAACACCTTTGTGTCTGGCAACTGTGATGTGGTGATTGGCATTGTGAAC
AACACAGTCTATGACCCACTCCAACCTGAACTGGACTCCTTCAAGGAGGAACTGGACAAATACTTCAA
GAACCACACCAGCCCTGATGTGGACCTGGGAGACATCTCTGGCATCAATGCCTCTGTGGTGAACATCC
AGAAGGAGATTGACAGACTGAATGAGGTGGCTAAGAACCTGAATGAGTCCCTGATTGACCTCCAAGAA
CTGGGCAAAtaagtttaaacccgctgatcagcctcgactgtgccttctagttgccagccatctgttgt
ttgcccctcccccgtgccttccttgaccctggaaggtgccactcccactgtcctttcctaataaaatg
aggaaattgcatcgcattgtctgagtaggtgtcattctattctggggggtggggtggggcaggacagc
aagggggaggattgggaagacaatagcaggcatgctggggatgcggtgggctctatggcttctgaggc
ggaaagaaccagctggggctctagggggtatccccacgcgccctgtagcggcgcattaagcgcggcgg
gtgtggtggttacgcgcagcgtgaccgctacacttgccagcgccctagcgcccgctcctttcgctttc
ttcccttcctttctcgccacgttcgccggctttccccgtcaagctctaaatcgggggctccctttagg
gttccgatttagtgctttacggcacctcgaccccaaaaaacttgattagggtgatggttcacgtagtg
ggccatcgccctgatagacggtttttcgccctttgacgttggagtccacgttctttaatagtggactc
ttgttccaaactggaacaacactcaaccctatctcggtctattcttttgatttataagggattttgcc
gatttcggcctattggttaaaaaatgagctgatttaacaaaaatttaacgcgaattaattctgtggaa
tgtgtgtcagttagggtgtggaaagtccccaggctccccagcaggcagaagtatgcaaagcatgcatc
tcaattagtcagcaaccaggtgtggaaagtccccaggctccccagcaggcagaagtatgcaaagcatg
catctcaattagtcagcaaccatagtcccgcccctaactccgcccatcccgcccctaactccgcccag
ttccgcccattctccgccccatggctgactaattttttttatttatgcagaggccgaggccgcctctg
cctctgagctattccagaagtagtgaggaggcttttttggaggcctaggcttttgcaaaaagctcccg
ggagcttgtatatccattttcggatctgatcaagagacaggatgaggatcgtttcgcatgattgaaca
agatggattgcacgcaggttctccggccgcttgggtggagaggctattcggctatgactgggcacaac
agacaatcggctgctctgatgccgccgtgttccggctgtcagcgcaggggcgcccggttctttttgtc
aagaccgacctgtccggtgccctgaatgaactgcaggacgaggcagcgcggctatcgtggctggccac
gacgggcgttccttgcgcagctgtgctcgacgttgtcactgaagcgggaagggactggctgctattgg
gcgaagtgccggggcaggatctcctgtcatctcaccttgctcctgccgagaaagtatccatcatggct
gatgcaatgcggcggctgcatacgcttgatccggctacctgcccattcgaccaccaagcgaaacatcg
catcgagcgagcacgtactcggatggaagccggtcttgtcgatcaggatgatctggacgaagagcatc
aggggctcgcgccagccgaactgttcgccaggctcaaggcgcgcatgcccgacggcgaggatctcgtc
gtgacccatggcgatgcctgcttgccgaatatcatggtggaaaatggccgcttttctggattcatcga
ctgtggccggctgggtgtggcggaccgctatcaggacatagcgttggctacccgtgatattgctgaag
agcttggcggcgaatgggctgaccgcttcctcgtgctttacggtatcgccgctcccgattcgcagcgc
atcgccttctatcgccttcttgacgagttcttctgagcgggactctggggttcgaaatgaccgaccaa
gcgacgcccaacctgccatcacgagatttcgattccaccgccgccttctatgaaaggttgggcttcgg
aatcgttttccgggacgccggctggatgatcctccagcgcggggatctcatgctggagttcttcgccc
accccaacttgtttattgcagcttataatggttacaaataaagcaatagcatcacaaatttcacaaat
aaagcatttttttcactgcattctagttgtggtttgtccaaactcatcaatgtatcttatcatgtctg
tataccgtcgacctctagctagagcttggcgtaatcatggtcatagctgtttcctgtgtgaaattgtt
atccgctcacaattccacacaacatacgagccggaagcataaagtgtaaagcctggggtgcctaatga
gtgagctaactcacattaattgcgttgcgctcactgcccgctttccagtcgggaaacctgtcgtgcca
gctgcattaatgaatcggccaacgcgcggggagaggcggtttgcgtattgggcgctcttccgcttcct
cgctcactgactcgctgcgctcggtcgttcggctgcggcgagcggtatcagctcactcaaaggcggta
atacggttatccacagaatcaggggataacgcaggaaagaacatgtgagcaaaaggccagcaaaaggc
caggaaccgtaaaaaggccgcgttgctggcgtttttccataggctccgcccccctgacgagcatcaca
aaaatcgacgctcaagtcagaggtggcgaaacccgacaggactataaagataccaggcgtttccccct
ggaagctccctcgtgcgctctcctgttccgaccctgccgcttaccggatacctgtccgcctttctccc
ttcgggaagcgtggcgctttctcatagctcacgctgtaggtatctcagttcggtgtaggtcgttcgct
ccaagctgggctgtgtgcacgaaccccccgttcagcccgaccgctgcgccttatccggtaactatcgt
cttgagtccaacccggtaagacacgacttatcgccactggcagcagccactggtaacaggattagcag
agcgaggtatgtaggcggtgctacagagttcttgaagtggtggcctaactacggctacactagaagaa
cagtatttggtatctgcgctctgctgaagccagttaccttcggaaaaagagttggtagctcttgatcc
ggcaaacaaaccaccgctggtagcggtttttttgtttgcaagcagcagattacgcgcagaaaaaaagg
atctcaagaagatcctttgatcttttctacggggtctgacgctcagtggaacgaaaactcacgttaag
ggattttggtcatgagattatcaaaaaggatcttcacctagatccttttaaattaaaaatgaagtttt
aaatcaatctaaagtatatatgagtaaacttggtctgacagttaccaatgcttaatcagtgaggcacc
tatctcagcgatctgtctatttcgttcatccatagttgcctgactccccgtcgtgtagataactacga
tacgggagggcttaccatctggccccagtgctgcaatgataccgcgagacccacgctcaccggctcca
gatttatcagcaataaaccagccagccggaagggccgagcgcagaagtggtcctgcaactttatccgc
ctccatccagtctattaattgttgccgggaagctagagtaagtagttcgccagttaatagtttgcgca
acgttgttgccattgctacaggcatcgtggtgtcacgctcgtcgtttggtatggcttcattcagctcc
ggttcccaacgatcaaggcgagttacatgatcccccatgttgtgcaaaaaagcggttagctccttcgg
tcctccgatcgttgtcagaagtaagttggccgcagtgttatcactcatggttatggcagcactgcata
attctcttactgtcatgccatccgtaagatgcttttctgtgactggtgagtactcaaccaagtcattc
tgagaatagtgtatgcggcgaccgagttgctcttgcccggcgtcaatacgggataataccgcgccaca
tagcagaactttaaaagtgctcatcattggaaaacgttcttcggggcgaaaactctcaaggatcttac
cgctgttgagatccagttcgatgtaacccactcgtgcacccaactgatcttcagcatcttttactttc
accagcgtttctgggtgagcaaaaacaggaaggcaaaatgccgcaaaaaagggaataagggcgacacg
gaaatgttgaatactcatactcttcctttttcaatattattgaagcatttatcagggttattgtctca
tgagcggatacatatttgaatgtatttagaaaaataaacaaataggggttccgcgcacatttccccga
aaagtgccacctgacgtc
pMD133_pcDNA3.1_SARS-CoV-2_stabilized-S-protein_mut_v3
SEQ ID NO: 110
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGAGGGCAAG
CAGGGCAACTTCAAGAACCTGAGGGAGTTTGTGTTCAAGAACATTGATGGCTACTTCAAGATTTACAG
CAAACACACACCAATCAACCTGGTGAGGGACCTGCCACAGGGCTTCTCTGCCTTGGAACCACTGGTGG
ACCTGCCAATTGGCATCAACATCACCAGGTTCCAGACCCTGCTGGCTCTGCACAGGTCCTACCTGACA
CCTGGAGACTCCTCCTCTGGCTGGACAGCAGGAGCAGCAGCCTACTATGTGGGCTACCTCCAACCAAG
GACCTTCCTGCTGAAATACAATGAGAATGGCACCATCACAGATGCTGTGGACTGTGCCCTGGACCCAC
TGTCTGAGACCAAGTGTACCCTGAAATCCTTCACAGTGGAGAAGGGCATCTACCAGACCAGCAACTTC
AGGGTCCAACCAACAGAGAGCATTGTGAGGTTTCCAAACATCACCAACCTGTGTCCATTTGGAGAGGT
GTTCAATGCCACCAGGTTTGCCTCTGTCTATGCCTGGAACAGGAAGAGGATTAGCAACTGTGTGGCTG
ACTACTCTGTGCTCTACAACTCTGCCTCCTTCAGCACCTTCAAGTGTTATGGAGTGAGCCCAACCAAA
CTGAATGACCTGTGTTTCACCAATGTCTATGCTGACTCCTTTGTGATTAGGGGAGATGAGGTGAGACA
GATTGCCCCTGGACAAACAGGCAAGATTGCTGACTACAACTACAAACTGCCTGATGACTTCACAGGCT
GTGTGATTGCCTGGAACAGCAACAACCTGGACAGCAAGGTGGGAGGCAACTACAACTACCTCTACAGA
CTGTTCAGGAAGAGCAACCTGAAACCATTTGAGAGGGACATCAGCACAGAGATTTACgctGCTGGCAG
CACACCATGTAATGGAGTGGAGGGCgccgctTGTTACTTTCCACTCCAATCCTATGGCTTCCAACCAA
CCAATGGAGTGGGCTACCAACCATACAGGGTGGTGGTGCTGTCCTTTGAACTGCTCCATGCCCCTGCC
ACAGTGTGTGGACCAAAGAAGAGCACCAACCTGGTGAAGAACAAGTGTGTGAACTTCAACTTCAATGG
ACTGACAGGCACAGGAGTGCTGACAGAGAGCAACAAGAAGTTCCTGCCATTCCAACAGTTTGGCAGGG
ACATTGCTGACACCACAGATGCTGTGAGGGACCCACAGACCTTGGAGATTCTGGACATCACACCATGT
TCCTTTGGAGGAGTGTCTGTGATTACACCTGGCACCAACACCAGCAACCAGGTGGCTGTGCTCTACCA
GGATGTGAACTGTACTGAGGTGCCTGTGGCTATCCATGCTGACCAACTTACACCAACCTGGAGGGTCT
ACAGCACAGGCAGCAATGTGTTCCAGACCAGGGCTGGCTGTCTGATTGGAGCAGAGCATGTGAACAAC
TCCTATGAGTGTGACATCCCAATTGGAGCAGGCATCTGTGCCTCCTACCAGACCCAGACCAACAGCCC
AAGGAGGGCAAGGTCTGTGGCAAGCCAGAGCATCATTGCCTACACAATGAGTCTGGGAGCAGAGAACT
CTGTGGCTTACAGCAACAACAGCATTGCCATCCCAACCAACTTCACCATCTCTGTGACCACAGAGATT
CTGCCTGTGAGTATGACCAAGACCTCTGTGGACTGTACAATGTATATCTGTGGAGACAGCACAGAGTG
TAGCAACCTGCTGCTCCAATATGGCTCCTTCTGTACCCAACTTAACAGGGCTCTGACAGGCATTGCTG
TGGAACAGGACAAGAACACCCAGGAGGTGTTTGCCCAGGTGAAGCAGATTTACAAGACACCTCCAATC
AAGGACTTTGGAGGCTTCAACTTCAGCCAGATTCTGCCTGACCCAAGCAAGCCAAGCAAGAGGTCCTT
CATTGAGGACCTGCTGTTCAACAAGGTGACCCTGGCTGATGCTGGCTTCATCAAGCAATATGGAGACT
GTCTGGGAGACATTGCTGCCAGGGACCTGATTTGTGCCCAGAAGTTCAATGGACTGACAGTGCTGCCT
CCACTGCTGACAGATGAGATGATTGCCCAATACACCTCTGCCCTGCTGGCTGGCACCATCACCTCTGG
CTGGACCTTTGGAGCAGGAGCAGCCCTCCAAATCCCATTTGCTATGCAGATGGCTTACAGGTTCAATG
GCATTGGAGTGACCCAGAATGTGCTCTATGAGAACCAGAAACTGATTGCCAACCAGTTCAACTCTGCC
ATTGGCAAGATTCAGGACTCCCTGTCCAGCACAGCCTCTGCCCTGGGCAAACTCCAAGATGTGGTGAA
CCAGAATGCCCAGGCTCTGAACACCCTGGTGAAGCAACTTTCCAGCAACTTTGGAGCCATCTCCTCTG
TGCTGAATGACATCCTGAGCAGACTGGACcctcccGAGGCTGAGGTCCAGATTGACAGACTGATTACA
GGCAGACTCCAATCCCTCCAAACCTATGTGACCCAACAACTTATCAGGGCTGCTGAGATTAGGGCATC
TGCCAACCTGGCTGCCACCAAGATGAGTGAGTGTGTGCTGGGACAAAGCAAGAGGGTGGACTTCTGTG
GCAAGGGCTACCACCTGATGAGTTTTCCACAGTCTGCCCCTCATGGAGTGGTGTTCCTGCATGTGACC
TATGTGCCTGCCCAGGAGAAGAACTTCACCACAGCCCCTGCCATCTGCCATGATGGCAAGGCTCACTT
TCCAAGGGAGGGAGTGTTTGTGAGCAATGGCACCCACTGGTTTGTGACCCAGAGGAACTTCTATGAAC
CACAGATTATCACCACAGACAACACCTTTGTGTCTGGCAACTGTGATGTGGTGATTGGCATTGTGAAC
AACACAGTCTATGACCCACTCCAACCTGAACTGGACTCCTTCAAGGAGGAACTGGACAAATACTTCAA
GAACCACACCAGCCCTGATGTGGACCTGGGAGACATCTCTGGCATCAATGCCTCTGTGGTGAACATCC
AGAAGGAGATTGACAGACTGAATGAGGTGGCTAAGAACCTGAATGAGTCCCTGATTGACCTCCAAGAA
CTGGGCAAAtaagtttaaacccgctgatcagcctcgactgtgccttctagttgccagccatctgttgt
ttgcccctcccccgtgccttccttgaccctggaaggtgccactcccactgtcctttcctaataaaatg
aggaaattgcatcgcattgtctgagtaggtgtcattctattctggggggtggggtggggcaggacagc
aagggggaggattgggaagacaatagcaggcatgctggggatgcggtgggctctatggcttctgaggc
ggaaagaaccagctggggctctagggggtatccccacgcgccctgtagcggcgcattaagcgcggcgg
gtgtggtggttacgcgcagcgtgaccgctacacttgccagcgccctagcgcccgctcctttcgctttc
ttcccttcctttctcgccacgttcgccggctttccccgtcaagctctaaatcgggggctccctttagg
gttccgatttagtgctttacggcacctcgaccccaaaaaacttgattagggtgatggttcacgtagtg
ggccatcgccctgatagacggtttttcgccctttgacgttggagtccacgttctttaatagtggactc
ttgttccaaactggaacaacactcaaccctatctcggtctattcttttgatttataagggattttgcc
gatttcggcctattggttaaaaaatgagctgatttaacaaaaatttaacgcgaattaattctgtggaa
tgtgtgtcagttagggtgtggaaagtccccaggctccccagcaggcagaagtatgcaaagcatgcatc
tcaattagtcagcaaccaggtgtggaaagtccccaggctccccagcaggcagaagtatgcaaagcatg
catctcaattagtcagcaaccatagtcccgcccctaactccgcccatcccgcccctaactccgcccag
ttccgcccattctccgccccatggctgactaattttttttatttatgcagaggccgaggccgcctctg
cctctgagctattccagaagtagtgaggaggcttttttggaggcctaggcttttgcaaaaagctcccg
ggagcttgtatatccattttcggatctgatcaagagacaggatgaggatcgtttcgcatgattgaaca
agatggattgcacgcaggttctccggccgcttgggtggagaggctattcggctatgactgggcacaac
agacaatcggctgctctgatgccgccgtgttccggctgtcagcgcaggggcgcccggttctttttgtc
aagaccgacctgtccggtgccctgaatgaactgcaggacgaggcagcgcggctatcgtggctggccac
gacgggcgttccttgcgcagctgtgctcgacgttgtcactgaagcgggaagggactggctgctattgg
gcgaagtgccggggcaggatctcctgtcatctcaccttgctcctgccgagaaagtatccatcatggct
gatgcaatgcggcggctgcatacgcttgatccggctacctgcccattcgaccaccaagcgaaacatcg
catcgagcgagcacgtactcggatggaagccggtcttgtcgatcaggatgatctggacgaagagcatc
aggggctcgcgccagccgaactgttcgccaggctcaaggcgcgcatgcccgacggcgaggatctcgtc
gtgacccatggcgatgcctgcttgccgaatatcatggtggaaaatggccgcttttctggattcatcga
ctgtggccggctgggtgtggcggaccgctatcaggacatagcgttggctacccgtgatattgctgaag
agcttggcggcgaatgggctgaccgcttcctcgtgctttacggtatcgccgctcccgattcgcagcgc
atcgccttctatcgccttcttgacgagttcttctgagcgggactctggggttcgaaatgaccgaccaa
gcgacgcccaacctgccatcacgagatttcgattccaccgccgccttctatgaaaggttgggcttcgg
aatcgttttccgggacgccggctggatgatcctccagcgcggggatctcatgctggagttcttcgccc
accccaacttgtttattgcagcttataatggttacaaataaagcaatagcatcacaaatttcacaaat
aaagcatttttttcactgcattctagttgtggtttgtccaaactcatcaatgtatcttatcatgtctg
tataccgtcgacctctagctagagcttggcgtaatcatggtcatagctgtttcctgtgtgaaattgtt
atccgctcacaattccacacaacatacgagccggaagcataaagtgtaaagcctggggtgcctaatga
gtgagctaactcacattaattgcgttgcgctcactgcccgctttccagtcgggaaacctgtcgtgcca
gctgcattaatgaatcggccaacgcgcggggagaggcggtttgcgtattgggcgctcttccgcttcct
cgctcactgactcgctgcgctcggtcgttcggctgcggcgagcggtatcagctcactcaaaggcggta
atacggttatccacagaatcaggggataacgcaggaaagaacatgtgagcaaaaggccagcaaaaggc
caggaaccgtaaaaaggccgcgttgctggcgtttttccataggctccgcccccctgacgagcatcaca
aaaatcgacgctcaagtcagaggtggcgaaacccgacaggactataaagataccaggcgtttccccct
ggaagctccctcgtgcgctctcctgttccgaccctgccgcttaccggatacctgtccgcctttctccc
ttcgggaagcgtggcgctttctcatagctcacgctgtaggtatctcagttcggtgtaggtcgttcgct
ccaagctgggctgtgtgcacgaaccccccgttcagcccgaccgctgcgccttatccggtaactatcgt
cttgagtccaacccggtaagacacgacttatcgccactggcagcagccactggtaacaggattagcag
agcgaggtatgtaggcggtgctacagagttcttgaagtggtggcctaactacggctacactagaagaa
cagtatttggtatctgcgctctgctgaagccagttaccttcggaaaaagagttggtagctcttgatcc
ggcaaacaaaccaccgctggtagcggtttttttgtttgcaagcagcagattacgcgcagaaaaaaagg
atctcaagaagatcctttgatcttttctacggggtctgacgctcagtggaacgaaaactcacgttaag
ggattttggtcatgagattatcaaaaaggatcttcacctagatccttttaaattaaaaatgaagtttt
aaatcaatctaaagtatatatgagtaaacttggtctgacagttaccaatgcttaatcagtgaggcacc
tatctcagcgatctgtctatttcgttcatccatagttgcctgactccccgtcgtgtagataactacga
tacgggagggcttaccatctggccccagtgctgcaatgataccgcgagacccacgctcaccggctcca
gatttatcagcaataaaccagccagccggaagggccgagcgcagaagtggtcctgcaactttatccgc
ctccatccagtctattaattgttgccgggaagctagagtaagtagttcgccagttaatagtttgcgca
acgttgttgccattgctacaggcatcgtggtgtcacgctcgtcgtttggtatggcttcattcagctcc
ggttcccaacgatcaaggcgagttacatgatcccccatgttgtgcaaaaaagcggttagctccttcgg
tcctccgatcgttgtcagaagtaagttggccgcagtgttatcactcatggttatggcagcactgcata
attctcttactgtcatgccatccgtaagatgcttttctgtgactggtgagtactcaaccaagtcattc
tgagaatagtgtatgcggcgaccgagttgctcttgcccggcgtcaatacgggataataccgcgccaca
tagcagaactttaaaagtgctcatcattggaaaacgttcttcggggcgaaaactctcaaggatcttac
cgctgttgagatccagttcgatgtaacccactcgtgcacccaactgatcttcagcatcttttactttc
accagcgtttctgggtgagcaaaaacaggaaggcaaaatgccgcaaaaaagggaataagggcgacacg
gaaatgttgaatactcatactcttcctttttcaatattattgaagcatttatcagggttattgtctca
tgagcggatacatatttgaatgtatttagaaaaataaacaaataggggttccgcgcacatttccccga
aaagtgccacctgacgtc
pMD134_pcDNA3.1_SARS-CoV-2_stabilized-S-protein_mut_v4
SEQ ID NO: 111
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGAGGGCAAG
CAGGGCAACTTCAAGAACCTGAGGGAGTTTGTGTTCAAGAACATTGATGGCTACTTCAAGATTTACAG
CAAACACACACCAATCAACCTGGTGAGGGACCTGCCACAGGGCTTCTCTGCCTTGGAACCACTGGTGG
ACCTGCCAATTGGCATCAACATCACCAGGTTCCAGACCCTGCTGGCTCTGCACAGGTCCTACCTGACA
CCTGGAGACTCCTCCTCTGGCTGGACAGCAGGAGCAGCAGCCTACTATGTGGGCTACCTCCAACCAAG
GACCTTCCTGCTGAAATACAATGAGAATGGCACCATCACAGATGCTGTGGACTGTGCCCTGGACCCAC
TGTCTGAGACCAAGTGTACCCTGAAATCCTTCACAGTGGAGAAGGGCATCTACCAGACCAGCAACTTC
AGGGTCCAACCAACAGAGAGCATTGTGAGGTTTCCAAACATCACCAACCTGTGTCCATTTGGAGAGGT
GTTCAATGCCACCAGGTTTGCCTCTGTCTATGCCTGGAACAGGAAGAGGATTAGCAACTGTGTGGCTG
ACTACTCTGTGCTCTACAACTCTGCCTCCTTCAGCACCTTCAAGTGTTATGGAGTGAGCCCAACCAAA
CTGAATGACCTGTGTTTCACCAATGTCTATGCTGACTCCTTTGTGATTAGGGGAGATGAGGTGAGACA
GATTGCCCCTGGACAAACAGGCgccATTGCTGACTACAACTACAAACTGCCTGATGACTTCACAGGCT
GTGTGATTGCCTGGAACAGCgccAACCTGGACAGCAAGGTGGGAGGCAACgccAACTACCTCTACAGA
gccgctAGGAAGAGCAACCTGAAACCATTTGAGAGGGACATCAGCACAGAGATTgccgctGCTGGCAG
CACACCATGTAATGGAGTGGAGGGCgccgctTGTTACTTTCCACTCgccTCCTATGGCTTCgccCCAg
ctgccGGAGTGGGCTACCAACCATACAGGGTGGTGGTGCTGTCCTTTGAACTGCTCCATGCCCCTGCC
ACAGTGTGTGGACCAAAGAAGAGCACCAACCTGGTGAAGAACAAGTGTGTGAACTTCAACgccAATGG
ACTGACAGGCACAGGAGTGCTGACAGAGAGCAACAAGAAGTTCCTGCCATTCCAACAGTTTGGCAGGG
ACATTGCTGACACCACAGATGCTGTGAGGGACCCACAGACCTTGGAGATTCTGGACATCACACCATGT
TCCTTTGGAGGAGTGTCTGTGATTACACCTGGCACCAACACCAGCAACCAGGTGGCTGTGCTCTACCA
GGATGTGAACTGTACTGAGGTGCCTGTGGCTATCCATGCTGACCAACTTACACCAACCTGGAGGGTCT
ACAGCACAGGCAGCAATGTGTTCCAGACCAGGGCTGGCTGTCTGATTGGAGCAGAGCATGTGAACAAC
TCCTATGAGTGTGACATCCCAATTGGAGCAGGCATCTGTGCCTCCTACCAGACCCAGACCAACAGCCC
AAGGAGGGCAAGGTCTGTGGCAAGCCAGAGCATCATTGCCTACACAATGAGTCTGGGAGCAGAGAACT
CTGTGGCTTACAGCAACAACAGCATTGCCATCCCAACCAACTTCACCATCTCTGTGACCACAGAGATT
CTGCCTGTGAGTATGACCAAGACCTCTGTGGACTGTACAATGTATATCTGTGGAGACAGCACAGAGTG
TAGCAACCTGCTGCTCCAATATGGCTCCTTCTGTACCCAACTTAACAGGGCTCTGACAGGCATTGCTG
TGGAACAGGACAAGAACACCCAGGAGGTGTTTGCCCAGGTGAAGCAGATTTACAAGACACCTCCAATC
AAGGACTTTGGAGGCTTCAACTTCAGCCAGATTCTGCCTGACCCAAGCAAGCCAAGCAAGAGGTCCTT
CATTGAGGACCTGCTGTTCAACAAGGTGACCCTGGCTGATGCTGGCTTCATCAAGCAATATGGAGACT
GTCTGGGAGACATTGCTGCCAGGGACCTGATTTGTGCCCAGAAGTTCAATGGACTGACAGTGCTGCCT
CCACTGCTGACAGATGAGATGATTGCCCAATACACCTCTGCCCTGCTGGCTGGCACCATCACCTCTGG
CTGGACCTTTGGAGCAGGAGCAGCCCTCCAAATCCCATTTGCTATGCAGATGGCTTACAGGTTCAATG
GCATTGGAGTGACCCAGAATGTGCTCTATGAGAACCAGAAACTGATTGCCAACCAGTTCAACTCTGCC
ATTGGCAAGATTCAGGACTCCCTGTCCAGCACAGCCTCTGCCCTGGGCAAACTCCAAGATGTGGTGAA
CCAGAATGCCCAGGCTCTGAACACCCTGGTGAAGCAACTTTCCAGCAACTTTGGAGCCATCTCCTCTG
TGCTGAATGACATCCTGAGCAGACTGGACcctcccGAGGCTGAGGTCCAGATTGACAGACTGATTACA
GGCAGACTCCAATCCCTCCAAACCTATGTGACCCAACAACTTATCAGGGCTGCTGAGATTAGGGCATC
TGCCAACCTGGCTGCCACCAAGATGAGTGAGTGTGTGCTGGGACAAAGCAAGAGGGTGGACTTCTGTG
GCAAGGGCTACCACCTGATGAGTTTTCCACAGTCTGCCCCTCATGGAGTGGTGTTCCTGCATGTGACC
TATGTGCCTGCCCAGGAGAAGAACTTCACCACAGCCCCTGCCATCTGCCATGATGGCAAGGCTCACTT
TCCAAGGGAGGGAGTGTTTGTGAGCAATGGCACCCACTGGTTTGTGACCCAGAGGAACTTCTATGAAC
CACAGATTATCACCACAGACAACACCTTTGTGTCTGGCAACTGTGATGTGGTGATTGGCATTGTGAAC
AACACAGTCTATGACCCACTCCAACCTGAACTGGACTCCTTCAAGGAGGAACTGGACAAATACTTCAA
GAACCACACCAGCCCTGATGTGGACCTGGGAGACATCTCTGGCATCAATGCCTCTGTGGTGAACATCC
AGAAGGAGATTGACAGACTGAATGAGGTGGCTAAGAACCTGAATGAGTCCCTGATTGACCTCCAAGAA
CTGGGCAAAtaagtttaaacccgctgatcagcctcgactgtgccttctagttgccagccatctgttgt
ttgcccctcccccgtgccttccttgaccctggaaggtgccactcccactgtcctttcctaataaaatg
aggaaattgcatcgcattgtctgagtaggtgtcattctattctggggggtggggtggggcaggacagc
aagggggaggattgggaagacaatagcaggcatgctggggatgcggtgggctctatggcttctgaggc
ggaaagaaccagctggggctctagggggtatccccacgcgccctgtagcggcgcattaagcgcggcgg
gtgtggtggttacgcgcagcgtgaccgctacacttgccagcgccctagcgcccgctcctttcgctttc
ttcccttcctttctcgccacgttcgccggctttccccgtcaagctctaaatcgggggctccctttagg
gttccgatttagtgctttacggcacctcgaccccaaaaaacttgattagggtgatggttcacgtagtg
ggccatcgccctgatagacggtttttcgccctttgacgttggagtccacgttctttaatagtggactc
ttgttccaaactggaacaacactcaaccctatctcggtctattcttttgatttataagggattttgcc
gatttcggcctattggttaaaaaatgagctgatttaacaaaaatttaacgcgaattaattctgtggaa
tgtgtgtcagttagggtgtggaaagtccccaggctccccagcaggcagaagtatgcaaagcatgcatc
tcaattagtcagcaaccaggtgtggaaagtccccaggctccccagcaggcagaagtatgcaaagcatg
catctcaattagtcagcaaccatagtcccgcccctaactccgcccatcccgcccctaactccgcccag
ttccgcccattctccgccccatggctgactaattttttttatttatgcagaggccgaggccgcctctg
cctctgagctattccagaagtagtgaggaggcttttttggaggcctaggcttttgcaaaaagctcccg
ggagcttgtatatccattttcggatctgatcaagagacaggatgaggatcgtttcgcatgattgaaca
agatggattgcacgcaggttctccggccgcttgggtggagaggctattcggctatgactgggcacaac
agacaatcggctgctctgatgccgccgtgttccggctgtcagcgcaggggcgcccggttctttttgtc
aagaccgacctgtccggtgccctgaatgaactgcaggacgaggcagcgcggctatcgtggctggccac
gacgggcgttccttgcgcagctgtgctcgacgttgtcactgaagcgggaagggactggctgctattgg
gcgaagtgccggggcaggatctcctgtcatctcaccttgctcctgccgagaaagtatccatcatggct
gatgcaatgcggcggctgcatacgcttgatccggctacctgcccattcgaccaccaagcgaaacatcg
catcgagcgagcacgtactcggatggaagccggtcttgtcgatcaggatgatctggacgaagagcatc
aggggctcgcgccagccgaactgttcgccaggctcaaggcgcgcatgcccgacggcgaggatctcgtc
gtgacccatggcgatgcctgcttgccgaatatcatggtggaaaatggccgcttttctggattcatcga
ctgtggccggctgggtgtggcggaccgctatcaggacatagcgttggctacccgtgatattgctgaag
agcttggcggcgaatgggctgaccgcttcctcgtgctttacggtatcgccgctcccgattcgcagcgc
atcgccttctatcgccttcttgacgagttcttctgagcgggactctggggttcgaaatgaccgaccaa
gcgacgcccaacctgccatcacgagatttcgattccaccgccgccttctatgaaaggttgggcttcgg
aatcgttttccgggacgccggctggatgatcctccagcgcggggatctcatgctggagttcttcgccc
accccaacttgtttattgcagcttataatggttacaaataaagcaatagcatcacaaatttcacaaat
aaagcatttttttcactgcattctagttgtggtttgtccaaactcatcaatgtatcttatcatgtctg
tataccgtcgacctctagctagagcttggcgtaatcatggtcatagctgtttcctgtgtgaaattgtt
atccgctcacaattccacacaacatacgagccggaagcataaagtgtaaagcctggggtgcctaatga
gtgagctaactcacattaattgcgttgcgctcactgcccgctttccagtcgggaaacctgtcgtgcca
gctgcattaatgaatcggccaacgcgcggggagaggcggtttgcgtattgggcgctcttccgcttcct
cgctcactgactcgctgcgctcggtcgttcggctgcggcgagcggtatcagctcactcaaaggcggta
atacggttatccacagaatcaggggataacgcaggaaagaacatgtgagcaaaaggccagcaaaaggc
caggaaccgtaaaaaggccgcgttgctggcgtttttccataggctccgcccccctgacgagcatcaca
aaaatcgacgctcaagtcagaggtggcgaaacccgacaggactataaagataccaggcgtttccccct
ggaagctccctcgtgcgctctcctgttccgaccctgccgcttaccggatacctgtccgcctttctccc
ttcgggaagcgtggcgctttctcatagctcacgctgtaggtatctcagttcggtgtaggtcgttcgct
ccaagctgggctgtgtgcacgaaccccccgttcagcccgaccgctgcgccttatccggtaactatcgt
cttgagtccaacccggtaagacacgacttatcgccactggcagcagccactggtaacaggattagcag
agcgaggtatgtaggcggtgctacagagttcttgaagtggtggcctaactacggctacactagaagaa
cagtatttggtatctgcgctctgctgaagccagttaccttcggaaaaagagttggtagctcttgatcc
ggcaaacaaaccaccgctggtagcggtttttttgtttgcaagcagcagattacgcgcagaaaaaaagg
atctcaagaagatcctttgatcttttctacggggtctgacgctcagtggaacgaaaactcacgttaag
ggattttggtcatgagattatcaaaaaggatcttcacctagatccttttaaattaaaaatgaagtttt
aaatcaatctaaagtatatatgagtaaacttggtctgacagttaccaatgcttaatcagtgaggcacc
tatctcagcgatctgtctatttcgttcatccatagttgcctgactccccgtcgtgtagataactacga
tacgggagggcttaccatctggccccagtgctgcaatgataccgcgagacccacgctcaccggctcca
gatttatcagcaataaaccagccagccggaagggccgagcgcagaagtggtcctgcaactttatccgc
ctccatccagtctattaattgttgccgggaagctagagtaagtagttcgccagttaatagtttgcgca
acgttgttgccattgctacaggcatcgtggtgtcacgctcgtcgtttggtatggcttcattcagctcc
ggttcccaacgatcaaggcgagttacatgatcccccatgttgtgcaaaaaagcggttagctccttcgg
tcctccgatcgttgtcagaagtaagttggccgcagtgttatcactcatggttatggcagcactgcata
attctcttactgtcatgccatccgtaagatgcttttctgtgactggtgagtactcaaccaagtcattc
tgagaatagtgtatgcggcgaccgagttgctcttgcccggcgtcaatacgggataataccgcgccaca
tagcagaactttaaaagtgctcatcattggaaaacgttcttcggggcgaaaactctcaaggatcttac
cgctgttgagatccagttcgatgtaacccactcgtgcacccaactgatcttcagcatcttttactttc
accagcgtttctgggtgagcaaaaacaggaaggcaaaatgccgcaaaaaagggaataagggcgacacg
gaaatgttgaatactcatactcttcctttttcaatattattgaagcatttatcagggttattgtctca
tgagcggatacatatttgaatgtatttagaaaaataaacaaataggggttccgcgcacatttccccga
aaagtgccacctgacgtc
pMD135_pcDNA3.1_SARS-CoV-2_stabilized-S-protein
SEQ ID NO: 112
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCAAGATTGCTGACTACAACTACAAACTGCCTGAT
GACTTCACAGGCTGTGTGATTGCCTGGAACAGCAACAACCTGGACAGCAAGGTGGGAGGCAACTACAA
CTACCTCTACAGACTGTTCAGGAAGAGCAACCTGAAACCATTTGAGAGGGACATCAGCACAGAGATTT
ACCAGGCTGGCAGCACACCATGTAATGGAGTGGAGGGCTTCAACTGTTACTTTCCACTCCAATCCTAT
GGCTTCCAACCAACCAATGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACcctcccGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAAtaagtttaaacccgctgatcagcctcgactgtgccttctagttgcc
agccatctgttgtttgcccctcccccgtgccttccttgaccctggaaggtgccactcccactgtcctt
tcctaataaaatgaggaaattgcatcgcattgtctgagtaggtgtcattctattctggggggtggggt
ggggcaggacagcaagggggaggattgggaagacaatagcaggcatgctggggatgcggtgggctcta
tggcttctgaggcggaaagaaccagctggggctctagggggtatccccacgcgccctgtagcggcgca
ttaagcgcggcgggtgtggtggttacgcgcagcgtgaccgctacacttgccagcgccctagcgcccgc
tcctttcgctttcttcccttcctttctcgccacgttcgccggctttccccgtcaagctctaaatcggg
ggctccctttagggttccgatttagtgctttacggcacctcgaccccaaaaaacttgattagggtgat
ggttcacgtagtgggccatcgccctgatagacggtttttcgccctttgacgttggagtccacgttctt
taatagtggactcttgttccaaactggaacaacactcaaccctatctcggtctattcttttgatttat
aagggattttgccgatttcggcctattggttaaaaaatgagctgatttaacaaaaatttaacgcgaat
taattctgtggaatgtgtgtcagttagggtgtggaaagtccccaggctccccagcaggcagaagtatg
caaagcatgcatctcaattagtcagcaaccaggtgtggaaagtccccaggctccccagcaggcagaag
tatgcaaagcatgcatctcaattagtcagcaaccatagtcccgcccctaactccgcccatcccgcccc
taactccgcccagttccgcccattctccgccccatggctgactaattttttttatttatgcagaggcc
gaggccgcctctgcctctgagctattccagaagtagtgaggaggcttttttggaggcctaggcttttg
caaaaagctcccgggagcttgtatatccattttcggatctgatcaagagacaggatgaggatcgtttc
gcatgattgaacaagatggattgcacgcaggttctccggccgcttgggtggagaggctattcggctat
gactgggcacaacagacaatcggctgctctgatgccgccgtgttccggctgtcagcgcaggggcgccc
ggttctttttgtcaagaccgacctgtccggtgccctgaatgaactgcaggacgaggcagcgcggctat
cgtggctggccacgacgggcgttccttgcgcagctgtgctcgacgttgtcactgaagcgggaagggac
tggctgctattgggcgaagtgccggggcaggatctcctgtcatctcaccttgctcctgccgagaaagt
atccatcatggctgatgcaatgcggcggctgcatacgcttgatccggctacctgcccattcgaccacc
aagcgaaacatcgcatcgagcgagcacgtactcggatggaagccggtcttgtcgatcaggatgatctg
gacgaagagcatcaggggctcgcgccagccgaactgttcgccaggctcaaggcgcgcatgcccgacgg
cgaggatctcgtcgtgacccatggcgatgcctgcttgccgaatatcatggtggaaaatggccgctttt
ctggattcatcgactgtggccggctgggtgtggcggaccgctatcaggacatagcgttggctacccgt
gatattgctgaagagcttggcggcgaatgggctgaccgcttcctcgtgctttacggtatcgccgctcc
cgattcgcagcgcatcgccttctatcgccttcttgacgagttcttctgagcgggactctggggttcga
aatgaccgaccaagcgacgcccaacctgccatcacgagatttcgattccaccgccgccttctatgaaa
ggttgggcttcggaatcgttttccgggacgccggctggatgatcctccagcgcggggatctcatgctg
gagttcttcgcccaccccaacttgtttattgcagcttataatggttacaaataaagcaatagcatcac
aaatttcacaaataaagcatttttttcactgcattctagttgtggtttgtccaaactcatcaatgtat
cttatcatgtctgtataccgtcgacctctagctagagcttggcgtaatcatggtcatagctgtttcct
gtgtgaaattgttatccgctcacaattccacacaacatacgagccggaagcataaagtgtaaagcctg
gggtgcctaatgagtgagctaactcacattaattgcgttgcgctcactgcccgctttccagtcgggaa
acctgtcgtgccagctgcattaatgaatcggccaacgcgcggggagaggcggtttgcgtattgggcgc
tcttccgcttcctcgctcactgactcgctgcgctcggtcgttcggctgcggcgagcggtatcagctca
ctcaaaggcggtaatacggttatccacagaatcaggggataacgcaggaaagaacatgtgagcaaaag
gccagcaaaaggccaggaaccgtaaaaaggccgcgttgctggcgtttttccataggctccgcccccct
gacgagcatcacaaaaatcgacgctcaagtcagaggtggcgaaacccgacaggactataaagatacca
ggcgtttccccctggaagctccctcgtgcgctctcctgttccgaccctgccgcttaccggatacctgt
ccgcctttctcccttcgggaagcgtggcgctttctcatagctcacgctgtaggtatctcagttcggtg
taggtcgttcgctccaagctgggctgtgtgcacgaaccccccgttcagcccgaccgctgcgccttatc
cggtaactatcgtcttgagtccaacccggtaagacacgacttatcgccactggcagcagccactggta
acaggattagcagagcgaggtatgtaggcggtgctacagagttcttgaagtggtggcctaactacggc
tacactagaagaacagtatttggtatctgcgctctgctgaagccagttaccttcggaaaaagagttgg
tagctcttgatccggcaaacaaaccaccgctggtagcggtttttttgtttgcaagcagcagattacgc
gcagaaaaaaaggatctcaagaagatcctttgatcttttctacggggtctgacgctcagtggaacgaa
aactcacgttaagggattttggtcatgagattatcaaaaaggatcttcacctagatccttttaaatta
aaaatgaagttttaaatcaatctaaagtatatatgagtaaacttggtctgacagttaccaatgcttaa
tcagtgaggcacctatctcagcgatctgtctatttcgttcatccatagttgcctgactccccgtcgtg
tagataactacgatacgggagggcttaccatctggccccagtgctgcaatgataccgcgagacccacg
ctcaccggctccagatttatcagcaataaaccagccagccggaagggccgagcgcagaagtggtcctg
caactttatccgcctccatccagtctattaattgttgccgggaagctagagtaagtagttcgccagtt
aatagtttgcgcaacgttgttgccattgctacaggcatcgtggtgtcacgctcgtcgtttggtatggc
ttcattcagctccggttcccaacgatcaaggcgagttacatgatcccccatgttgtgcaaaaaagcgg
ttagctccttcggtcctccgatcgttgtcagaagtaagttggccgcagtgttatcactcatggttatg
gcagcactgcataattctcttactgtcatgccatccgtaagatgcttttctgtgactggtgagtactc
aaccaagtcattctgagaatagtgtatgcggcgaccgagttgctcttgcccggcgtcaatacgggata
ataccgcgccacatagcagaactttaaaagtgctcatcattggaaaacgttcttcggggcgaaaactc
tcaaggatcttaccgctgttgagatccagttcgatgtaacccactcgtgcacccaactgatcttcagc
atcttttactttcaccagcgtttctgggtgagcaaaaacaggaaggcaaaatgccgcaaaaaagggaa
taagggcgacacggaaatgttgaatactcatactcttcctttttcaatattattgaagcatttatcag
ggttattgtctcatgagcggatacatatttgaatgtatttagaaaaataaacaaataggggttccgcg
cacatttccccgaaaagtgccacctgacgtc
pMD136-pcDNA3.1-SARS2-GGSG-foldon-G-6xHis-streptavidin-T2A-
mScarlet
SEQ ID NO: 113
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCAAGATTGCTGACTACAACTACAAACTGCCTGAT
GACTTCACAGGCTGTGTGATTGCCTGGAACAGCAACAACCTGGACAGCAAGGTGGGAGGCAACTACAA
CTACCTCTACAGACTGTTCAGGAAGAGCAACCTGAAACCATTTGAGAGGGACATCAGCACAGAGATTT
ACCAGGCTGGCAGCACACCATGTAATGGAGTGGAGGGCTTCAACTGTTACTTTCCACTCCAATCCTAT
GGCTTCCAACCAACCAATGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACcctcccGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAAggcggaggcagcgggTATATTCCGGAGGCTCCAAGAGACGGTCAGG
CGTACGTACGGAAGGACGGCGAATGGGTTCTCTTGAGCACTTTCTTAggcCATCATCATCACCACCAT
ATGGCGGAAGCTGGCATTACGGGGACCTGGTACAACCAACTTGGTTCAACATTCATTGTGACTGCGGG
AGCCGACGGAGCCCTCACGGGCACCTATGAGTCCGCCGTTGGCAATGCGGAGTCTCGCTATGTCCTGA
CAGGTAGATATGACAGCGCACCGGCTACGGACGGAAGTGGTACTGCTCTCGGTTGGACCGTGGCCTGG
AAAAACAATTATAGGAACGCACATAGCGCGACGACCTGGAGTGGACAATATGTCGGCGGCGCTGAAGC
CAGGATAAACACCCAATGGCTTCTCACCTCCGGAACAACTGAAGCAAACGCGTGGAAAAGCACTCTTG
TAGGCCACGACACGTTCACGAAGGTAAAGCCGAGCGCCGCATCCTATGAACAAggcagtggagagggc
agaggaagtctgctaacatgcggtgacgtcgaggagaatcctggcccaatgagtaaaggagaagctgt
gattaaagagttcatgcgcttcaaagttcacatggagggttctatgaacggtcacgagttcgagatcg
aaggcgaaggcgagggccgtccgtatgaaggcacccagaccgccaaactgaaagtgactaaaggcggc
ccgctgcctttttcctgggacatcctgagcccgcaatttatgtacggttctagggcgttcaccaaaca
cccagcggatatcccggactattataagcagtcttttccggaaggtttcaagtgggaacgcgtaatga
attttgaagatggtggtgccgtgaccgtcactcaggacacctccctggaggatggcaccctgatctat
aaagttaaactgcgtggtactaattttccacctgatggcccggtgatgcagaaaaagacgatgggttg
ggaggcgtctaccgaacgcttgtatccggaagatggtgtgctgaaaggcgacattaaaatggccctgc
gcctgaaagatggcggccgctatctggctgacttcaaaaccacgtacaaagccaagaaacctgtgcag
atgcctggcgcgtacaatgtggaccgcaaactggacatcacctctcataatgaagattatacggtggt
agagcaatatgagcgctccgagggtcgtcattctaccggtggcatggatgaactatacaaataagttt
aaacccgctgatcagcctcgactgtgccttctagttgccagccatctgttgtttgcccctcccccgtg
ccttccttgaccctggaaggtgccactcccactgtcctttcctaataaaatgaggaaattgcatcgca
ttgtctgagtaggtgtcattctattctggggggtggggtggggcaggacagcaagggggaggattggg
aagacaatagcaggcatgctggggatgcggtgggctctatggcttctgaggcggaaagaaccagctgg
ggctctagggggtatccccacgcgccctgtagcggcgcattaagcgcggcgggtgtggtggttacgcg
cagcgtgaccgctacacttgccagcgccctagcgcccgctcctttcgctttcttcccttcctttctcg
ccacgttcgccggctttccccgtcaagctctaaatcgggggctccctttagggttccgatttagtgct
ttacggcacctcgaccccaaaaaacttgattagggtgatggttcacgtagtgggccatcgccctgata
gacggtttttcgccctttgacgttggagtccacgttctttaatagtggactcttgttccaaactggaa
caacactcaaccctatctcggtctattcttttgatttataagggattttgccgatttcggcctattgg
ttaaaaaatgagctgatttaacaaaaatttaacgcgaattaattctgtggaatgtgtgtcagttaggg
tgtggaaagtccccaggctccccagcaggcagaagtatgcaaagcatgcatctcaattagtcagcaac
caggtgtggaaagtccccaggctccccagcaggcagaagtatgcaaagcatgcatctcaattagtcag
caaccatagtcccgcccctaactccgcccatcccgcccctaactccgcccagttccgcccattctccg
ccccatggctgactaattttttttatttatgcagaggccgaggccgcctctgcctctgagctattcca
gaagtagtgaggaggcttttttggaggcctaggcttttgcaaaaagctcccgggagcttgtatatcca
ttttcggatctgatcaagagacaggatgaggatcgtttcgcatgattgaacaagatggattgcacgca
ggttctccggccgcttgggtggagaggctattcggctatgactgggcacaacagacaatcggctgctc
tgatgccgccgtgttccggctgtcagcgcaggggcgcccggttctttttgtcaagaccgacctgtccg
gtgccctgaatgaactgcaggacgaggcagcgcggctatcgtggctggccacgacgggcgttccttgc
gcagctgtgctcgacgttgtcactgaagcgggaagggactggctgctattgggcgaagtgccggggca
ggatctcctgtcatctcaccttgctcctgccgagaaagtatccatcatggctgatgcaatgcggcggc
tgcatacgcttgatccggctacctgcccattcgaccaccaagcgaaacatcgcatcgagcgagcacgt
actcggatggaagccggtcttgtcgatcaggatgatctggacgaagagcatcaggggctcgcgccagc
cgaactgttcgccaggctcaaggcgcgcatgcccgacggcgaggatctcgtcgtgacccatggcgatg
cctgcttgccgaatatcatggtggaaaatggccgcttttctggattcatcgactgtggccggctgggt
gtggcggaccgctatcaggacatagcgttggctacccgtgatattgctgaagagcttggcggcgaatg
ggctgaccgcttcctcgtgctttacggtatcgccgctcccgattcgcagcgcatcgccttctatcgcc
ttcttgacgagttcttctgagcgggactctggggttcgaaatgaccgaccaagcgacgcccaacctgc
catcacgagatttcgattccaccgccgccttctatgaaaggttgggcttcggaatcgttttccgggac
gccggctggatgatcctccagcgcggggatctcatgctggagttcttcgcccaccccaacttgtttat
tgcagcttataatggttacaaataaagcaatagcatcacaaatttcacaaataaagcatttttttcac
tgcattctagttgtggtttgtccaaactcatcaatgtatcttatcatgtctgtataccgtcgacctct
agctagagcttggcgtaatcatggtcatagctgtttcctgtgtgaaattgttatccgctcacaattcc
acacaacatacgagccggaagcataaagtgtaaagcctggggtgcctaatgagtgagctaactcacat
taattgcgttgcgctcactgcccgctttccagtcgggaaacctgtcgtgccagctgcattaatgaatc
ggccaacgcgcggggagaggcggtttgcgtattgggcgctcttccgcttcctcgctcactgactcgct
gcgctcggtcgttcggctgcggcgagcggtatcagctcactcaaaggcggtaatacggttatccacag
aatcaggggataacgcaggaaagaacatgtgagcaaaaggccagcaaaaggccaggaaccgtaaaaag
gccgcgttgctggcgtttttccataggctccgcccccctgacgagcatcacaaaaatcgacgctcaag
tcagaggtggcgaaacccgacaggactataaagataccaggcgtttccccctggaagctccctcgtgc
gctctcctgttccgaccctgccgcttaccggatacctgtccgcctttctcccttcgggaagcgtggcg
ctttctcatagctcacgctgtaggtatctcagttcggtgtaggtcgttcgctccaagctgggctgtgt
gcacgaaccccccgttcagcccgaccgctgcgccttatccggtaactatcgtcttgagtccaacccgg
taagacacgacttatcgccactggcagcagccactggtaacaggattagcagagcgaggtatgtaggc
ggtgctacagagttcttgaagtggtggcctaactacggctacactagaagaacagtatttggtatctg
cgctctgctgaagccagttaccttcggaaaaagagttggtagctcttgatccggcaaacaaaccaccg
ctggtagcggtttttttgtttgcaagcagcagattacgcgcagaaaaaaaggatctcaagaagatcct
ttgatcttttctacggggtctgacgctcagtggaacgaaaactcacgttaagggattttggtcatgag
attatcaaaaaggatcttcacctagatccttttaaattaaaaatgaagttttaaatcaatctaaagta
tatatgagtaaacttggtctgacagttaccaatgcttaatcagtgaggcacctatctcagcgatctgt
ctatttcgttcatccatagttgcctgactccccgtcgtgtagataactacgatacgggagggcttacc
atctggccccagtgctgcaatgataccgcgagacccacgctcaccggctccagatttatcagcaataa
accagccagccggaagggccgagcgcagaagtggtcctgcaactttatccgcctccatccagtctatt
aattgttgccgggaagctagagtaagtagttcgccagttaatagtttgcgcaacgttgttgccattgc
tacaggcatcgtggtgtcacgctcgtcgtttggtatggcttcattcagctccggttcccaacgatcaa
ggcgagttacatgatcccccatgttgtgcaaaaaagcggttagctccttcggtcctccgatcgttgtc
agaagtaagttggccgcagtgttatcactcatggttatggcagcactgcataattctcttactgtcat
gccatccgtaagatgcttttctgtgactggtgagtactcaaccaagtcattctgagaatagtgtatgc
ggcgaccgagttgctcttgcccggcgtcaatacgggataataccgcgccacatagcagaactttaaaa
gtgctcatcattggaaaacgttcttcggggcgaaaactctcaaggatcttaccgctgttgagatccag
ttcgatgtaacccactcgtgcacccaactgatcttcagcatcttttactttcaccagcgtttctgggt
gagcaaaaacaggaaggcaaaatgccgcaaaaaagggaataagggcgacacggaaatgttgaatactc
atactcttcctttttcaatattattgaagcatttatcagggttattgtctcatgagcggatacatatt
tgaatgtatttagaaaaataaacaaataggggttccgcgcacatttccccgaaaagtgccacctgacg
tc
pMD137-pcDNA3.1-SARS2-GGSG-foldon-G-6xHis-BirA-T2A-mScarlet
SEQ ID NO: 114
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGTGGACTGT
GCCCTGGACCCACTGTCTGAGACCAAGTGTACCCTGAAATCCTTCACAGTGGAGAAGGGCATCTACCA
GACCAGCAACTTCAGGGTCCAACCAACAGAGAGCATTGTGAGGTTTCCAAACATCACCAACCTGTGTC
CATTTGGAGAGGTGTTCAATGCCACCAGGTTTGCCTCTGTCTATGCCTGGAACAGGAAGAGGATTAGC
AACTGTGTGGCTGACTACTCTGTGCTCTACAACTCTGCCTCCTTCAGCACCTTCAAGTGTTATGGAGT
GAGCCCAACCAAACTGAATGACCTGTGTTTCACCAATGTCTATGCTGACTCCTTTGTGATTAGGGGAG
ATGAGGTGAGACAGATTGCCCCTGGACAAACAGGCAAGATTGCTGACTACAACTACAAACTGCCTGAT
GACTTCACAGGCTGTGTGATTGCCTGGAACAGCAACAACCTGGACAGCAAGGTGGGAGGCAACTACAA
CTACCTCTACAGACTGTTCAGGAAGAGCAACCTGAAACCATTTGAGAGGGACATCAGCACAGAGATTT
ACCAGGCTGGCAGCACACCATGTAATGGAGTGGAGGGCTTCAACTGTTACTTTCCACTCCAATCCTAT
GGCTTCCAACCAACCAATGGAGTGGGCTACCAACCATACAGGGTGGTGGTGCTGTCCTTTGAACTGCT
CCATGCCCCTGCCACAGTGTGTGGACCAAAGAAGAGCACCAACCTGGTGAAGAACAAGTGTGTGAACT
TCAACTTCAATGGACTGACAGGCACAGGAGTGCTGACAGAGAGCAACAAGAAGTTCCTGCCATTCCAA
CAGTTTGGCAGGGACATTGCTGACACCACAGATGCTGTGAGGGACCCACAGACCTTGGAGATTCTGGA
CATCACACCATGTTCCTTTGGAGGAGTGTCTGTGATTACACCTGGCACCAACACCAGCAACCAGGTGG
CTGTGCTCTACCAGGATGTGAACTGTACTGAGGTGCCTGTGGCTATCCATGCTGACCAACTTACACCA
ACCTGGAGGGTCTACAGCACAGGCAGCAATGTGTTCCAGACCAGGGCTGGCTGTCTGATTGGAGCAGA
GCATGTGAACAACTCCTATGAGTGTGACATCCCAATTGGAGCAGGCATCTGTGCCTCCTACCAGACCC
AGACCAACAGCCCAAGGAGGGCAAGGTCTGTGGCAAGCCAGAGCATCATTGCCTACACAATGAGTCTG
GGAGCAGAGAACTCTGTGGCTTACAGCAACAACAGCATTGCCATCCCAACCAACTTCACCATCTCTGT
GACCACAGAGATTCTGCCTGTGAGTATGACCAAGACCTCTGTGGACTGTACAATGTATATCTGTGGAG
ACAGCACAGAGTGTAGCAACCTGCTGCTCCAATATGGCTCCTTCTGTACCCAACTTAACAGGGCTCTG
ACAGGCATTGCTGTGGAACAGGACAAGAACACCCAGGAGGTGTTTGCCCAGGTGAAGCAGATTTACAA
GACACCTCCAATCAAGGACTTTGGAGGCTTCAACTTCAGCCAGATTCTGCCTGACCCAAGCAAGCCAA
GCAAGAGGTCCTTCATTGAGGACCTGCTGTTCAACAAGGTGACCCTGGCTGATGCTGGCTTCATCAAG
CAATATGGAGACTGTCTGGGAGACATTGCTGCCAGGGACCTGATTTGTGCCCAGAAGTTCAATGGACT
GACAGTGCTGCCTCCACTGCTGACAGATGAGATGATTGCCCAATACACCTCTGCCCTGCTGGCTGGCA
CCATCACCTCTGGCTGGACCTTTGGAGCAGGAGCAGCCCTCCAAATCCCATTTGCTATGCAGATGGCT
TACAGGTTCAATGGCATTGGAGTGACCCAGAATGTGCTCTATGAGAACCAGAAACTGATTGCCAACCA
GTTCAACTCTGCCATTGGCAAGATTCAGGACTCCCTGTCCAGCACAGCCTCTGCCCTGGGCAAACTCC
AAGATGTGGTGAACCAGAATGCCCAGGCTCTGAACACCCTGGTGAAGCAACTTTCCAGCAACTTTGGA
GCCATCTCCTCTGTGCTGAATGACATCCTGAGCAGACTGGACcctcccGAGGCTGAGGTCCAGATTGA
CAGACTGATTACAGGCAGACTCCAATCCCTCCAAACCTATGTGACCCAACAACTTATCAGGGCTGCTG
AGATTAGGGCATCTGCCAACCTGGCTGCCACCAAGATGAGTGAGTGTGTGCTGGGACAAAGCAAGAGG
GTGGACTTCTGTGGCAAGGGCTACCACCTGATGAGTTTTCCACAGTCTGCCCCTCATGGAGTGGTGTT
CCTGCATGTGACCTATGTGCCTGCCCAGGAGAAGAACTTCACCACAGCCCCTGCCATCTGCCATGATG
GCAAGGCTCACTTTCCAAGGGAGGGAGTGTTTGTGAGCAATGGCACCCACTGGTTTGTGACCCAGAGG
AACTTCTATGAACCACAGATTATCACCACAGACAACACCTTTGTGTCTGGCAACTGTGATGTGGTGAT
TGGCATTGTGAACAACACAGTCTATGACCCACTCCAACCTGAACTGGACTCCTTCAAGGAGGAACTGG
ACAAATACTTCAAGAACCACACCAGCCCTGATGTGGACCTGGGAGACATCTCTGGCATCAATGCCTCT
GTGGTGAACATCCAGAAGGAGATTGACAGACTGAATGAGGTGGCTAAGAACCTGAATGAGTCCCTGAT
TGACCTCCAAGAACTGGGCAAAggcggaggcagcgggTATATTCCGGAGGCTCCAAGAGACGGTCAGG
CGTACGTACGGAAGGACGGCGAATGGGTTCTCTTGAGCACTTTCTTAggcCATCATCATCACCACCAT
ATGAAGGACAACACAGTCCCGCTGAAACTTATTGCGCTTCTCGCAAATGGAGAGTTCCACTCTGGAGA
GCAACTCGGCGAGACCCTGGGAATGAGTCGGGCGGCTATCAACAAGCATATACAGACTTTGAGGGATT
GGGGTGTGGATGTCTTCACTGTTCCGGGTAAGGGCTATTCACTGCCCGAACCGATCCAACTTCTCAAT
GCCAAACAGATATTGGGGCAACTTGATGGTGGCTCTGTCGCCGTGTTGCCGGTCATCGATAGCACGAA
CCAATATCTCTTGGACCGCATCGGAGAACTCAAGAGCGGAGACGCATGTATAGCGGAATATCAGCAAG
CCGGTAGAGGAAGAAGGGGACGGAAATGGTTCTCTCCATTCGGAGCCAACCTGTATTTGAGCATGTTC
TGGCGATTGGAGCAGGGACCAGCCGCCGCCATCGGTCTCTCTCTTGTCATAGGTATTGTCATGGCGGA
AGTCCTCCGCAAACTCGGGGCCGACAAAGTACGCGTGAAATGGCCGAACGACCTCTATCTTCAGGATA
GGAAATTGGCAGGGATATTGGTTGAACTGACGGGTAAGACGGGGGACGCCGCTCAGATTGTTATAGGT
GCCGGAATAAACATGGCCATGCGCAGAGTTGAAGAGTCAGTGGTAAACCAGGGTTGGATCACTCTCCA
AGAGGCGGGAATAAACCTGGATCGCAACACACTGGCTGCCATGCTTATACGGGAACTCCGAGCTGCAT
TGGAACTGTTCGAGCAAGAAGGGCTTGCTCCGTACTTGAGCAGGTGGGAAAAGCTGGATAATTTCATA
AATCGCCCTGTTAAACTCATCATCGGAGATAAAGAGATTTTTGGCATAAGTCGAGGAATCGATAAACA
AGGTGCATTGCTGCTTGAGCAAGACGGTATAATCAAACCTTGGATGGGTGGCGAAATCTCACTTAGAA
GCGCGGAAAAATATGAACAAggcagtggagagggcagaggaagtctgctaacatgcggtgacgtcgag
gagaatcctggcccaatgagtaaaggagaagctgtgattaaagagttcatgcgcttcaaagttcacat
ggagggttctatgaacggtcacgagttcgagatcgaaggcgaaggcgagggccgtccgtatgaaggca
cccagaccgccaaactgaaagtgactaaaggcggcccgctgcctttttcctgggacatcctgagcccg
caatttatgtacggttctagggcgttcaccaaacacccagcggatatcccggactattataagcagtc
ttttccggaaggtttcaagtgggaacgcgtaatgaattttgaagatggtggtgccgtgaccgtcactc
aggacacctccctggaggatggcaccctgatctataaagttaaactgcgtggtactaattttccacct
gatggcccggtgatgcagaaaaagacgatgggttgggaggcgtctaccgaacgcttgtatccggaaga
tggtgtgctgaaaggcgacattaaaatggccctgcgcctgaaagatggcggccgctatctggctgact
tcaaaaccacgtacaaagccaagaaacctgtgcagatgcctggcgcgtacaatgtggaccgcaaactg
gacatcacctctcataatgaagattatacggtggtagagcaatatgagcgctccgagggtcgtcattc
taccggtggcatggatgaactatacaaataagtttaaacccgctgatcagcctcgactgtgccttcta
gttgccagccatctgttgtttgcccctcccccgtgccttccttgaccctggaaggtgccactcccact
gtcctttcctaataaaatgaggaaattgcatcgcattgtctgagtaggtgtcattctattctgggggg
tggggtggggcaggacagcaagggggaggattgggaagacaatagcaggcatgctggggatgcggtgg
gctctatggcttctgaggcggaaagaaccagctggggctctagggggtatccccacgcgccctgtagc
ggcgcattaagcgcggcgggtgtggtggttacgcgcagcgtgaccgctacacttgccagcgccctagc
gcccgctcctttcgctttcttcccttcctttctcgccacgttcgccggctttccccgtcaagctctaa
atcgggggctccctttagggttccgatttagtgctttacggcacctcgaccccaaaaaacttgattag
ggtgatggttcacgtagtgggccatcgccctgatagacggtttttcgccctttgacgttggagtccac
gttctttaatagtggactcttgttccaaactggaacaacactcaaccctatctcggtctattcttttg
atttataagggattttgccgatttcggcctattggttaaaaaatgagctgatttaacaaaaatttaac
gcgaattaattctgtggaatgtgtgtcagttagggtgtggaaagtccccaggctccccagcaggcaga
agtatgcaaagcatgcatctcaattagtcagcaaccaggtgtggaaagtccccaggctccccagcagg
cagaagtatgcaaagcatgcatctcaattagtcagcaaccatagtcccgcccctaactccgcccatcc
cgcccctaactccgcccagttccgcccattctccgccccatggctgactaattttttttatttatgca
gaggccgaggccgcctctgcctctgagctattccagaagtagtgaggaggcttttttggaggcctagg
cttttgcaaaaagctcccgggagcttgtatatccattttcggatctgatcaagagacaggatgaggat
cgtttcgcatgattgaacaagatggattgcacgcaggttctccggccgcttgggtggagaggctattc
ggctatgactgggcacaacagacaatcggctgctctgatgccgccgtgttccggctgtcagcgcaggg
gcgcccggttctttttgtcaagaccgacctgtccggtgccctgaatgaactgcaggacgaggcagcgc
ggctatcgtggctggccacgacgggcgttccttgcgcagctgtgctcgacgttgtcactgaagcggga
agggactggctgctattgggcgaagtgccggggcaggatctcctgtcatctcaccttgctcctgccga
gaaagtatccatcatggctgatgcaatgcggcggctgcatacgcttgatccggctacctgcccattcg
accaccaagcgaaacatcgcatcgagcgagcacgtactcggatggaagccggtcttgtcgatcaggat
gatctggacgaagagcatcaggggctcgcgccagccgaactgttcgccaggctcaaggcgcgcatgcc
cgacggcgaggatctcgtcgtgacccatggcgatgcctgcttgccgaatatcatggtggaaaatggcc
gcttttctggattcatcgactgtggccggctgggtgtggcggaccgctatcaggacatagcgttggct
acccgtgatattgctgaagagcttggcggcgaatgggctgaccgcttcctcgtgctttacggtatcgc
cgctcccgattcgcagcgcatcgccttctatcgccttcttgacgagttcttctgagcgggactctggg
gttcgaaatgaccgaccaagcgacgcccaacctgccatcacgagatttcgattccaccgccgccttct
atgaaaggttgggcttcggaatcgttttccgggacgccggctggatgatcctccagcgcggggatctc
atgctggagttcttcgcccaccccaacttgtttattgcagcttataatggttacaaataaagcaatag
catcacaaatttcacaaataaagcatttttttcactgcattctagttgtggtttgtccaaactcatca
atgtatcttatcatgtctgtataccgtcgacctctagctagagcttggcgtaatcatggtcatagctg
tttcctgtgtgaaattgttatccgctcacaattccacacaacatacgagccggaagcataaagtgtaa
agcctggggtgcctaatgagtgagctaactcacattaattgcgttgcgctcactgcccgctttccagt
cgggaaacctgtcgtgccagctgcattaatgaatcggccaacgcgcggggagaggcggtttgcgtatt
gggcgctcttccgcttcctcgctcactgactcgctgcgctcggtcgttcggctgcggcgagcggtatc
agctcactcaaaggcggtaatacggttatccacagaatcaggggataacgcaggaaagaacatgtgag
caaaaggccagcaaaaggccaggaaccgtaaaaaggccgcgttgctggcgtttttccataggctccgc
ccccctgacgagcatcacaaaaatcgacgctcaagtcagaggtggcgaaacccgacaggactataaag
ataccaggcgtttccccctggaagctccctcgtgcgctctcctgttccgaccctgccgcttaccggat
acctgtccgcctttctcccttcgggaagcgtggcgctttctcatagctcacgctgtaggtatctcagt
tcggtgtaggtcgttcgctccaagctgggctgtgtgcacgaaccccccgttcagcccgaccgctgcgc
cttatccggtaactatcgtcttgagtccaacccggtaagacacgacttatcgccactggcagcagcca
ctggtaacaggattagcagagcgaggtatgtaggcggtgctacagagttcttgaagtggtggcctaac
tacggctacactagaagaacagtatttggtatctgcgctctgctgaagccagttaccttcggaaaaag
agttggtagctcttgatccggcaaacaaaccaccgctggtagcggtttttttgtttgcaagcagcaga
ttacgcgcagaaaaaaaggatctcaagaagatcctttgatcttttctacggggtctgacgctcagtgg
aacgaaaactcacgttaagggattttggtcatgagattatcaaaaaggatcttcacctagatcctttt
aaattaaaaatgaagttttaaatcaatctaaagtatatatgagtaaacttggtctgacagttaccaat
gcttaatcagtgaggcacctatctcagcgatctgtctatttcgttcatccatagttgcctgactcccc
gtcgtgtagataactacgatacgggagggcttaccatctggccccagtgctgcaatgataccgcgaga
cccacgctcaccggctccagatttatcagcaataaaccagccagccggaagggccgagcgcagaagtg
gtcctgcaactttatccgcctccatccagtctattaattgttgccgggaagctagagtaagtagttcg
ccagttaatagtttgcgcaacgttgttgccattgctacaggcatcgtggtgtcacgctcgtcgtttgg
tatggcttcattcagctccggttcccaacgatcaaggcgagttacatgatcccccatgttgtgcaaaa
aagcggttagctccttcggtcctccgatcgttgtcagaagtaagttggccgcagtgttatcactcatg
gttatggcagcactgcataattctcttactgtcatgccatccgtaagatgcttttctgtgactggtga
gtactcaaccaagtcattctgagaatagtgtatgcggcgaccgagttgctcttgcccggcgtcaatac
gggataataccgcgccacatagcagaactttaaaagtgctcatcattggaaaacgttcttcggggcga
aaactctcaaggatcttaccgctgttgagatccagttcgatgtaacccactcgtgcacccaactgatc
ttcagcatcttttactttcaccagcgtttctgggtgagcaaaaacaggaaggcaaaatgccgcaaaaa
agggaataagggcgacacggaaatgttgaatactcatactcttcctttttcaatattattgaagcatt
tatcagggttattgtctcatgagcggatacatatttgaatgtatttagaaaaataaacaaataggggt
tccgcgcacatttccccgaaaagtgccacctgacgtc
pMD138-pcDNA3.1-SARS2_mut_v1-GGSG-foldon-G-6xHis-BirA-T2A-mScarlet
SEQ ID NO: 115
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGAGGGCAAG
CAGGGCAACTTCAAGAACCTGAGGGAGTTTGTGTTCAAGAACATTGATGGCTACTTCAAGATTTACAG
CAAACACACACCAATCAACCTGGTGAGGGACCTGCCACAGGGCTTCTCTGCCTTGGAACCACTGGTGG
ACCTGCCAATTGGCATCAACATCACCAGGTTCCAGACCCTGCTGGCTCTGCACAGGTCCTACCTGACA
CCTGGAGACTCCTCCTCTGGCTGGACAGCAGGAGCAGCAGCCTACTATGTGGGCTACCTCCAACCAAG
GACCTTCCTGCTGAAATACAATGAGAATGGCACCATCACAGATGCTGTGGACTGTGCCCTGGACCCAC
TGTCTGAGACCAAGTGTACCCTGAAATCCTTCACAGTGGAGAAGGGCATCTACCAGACCAGCAACTTC
AGGGTCCAACCAACAGAGAGCATTGTGAGGTTTCCAAACATCACCAACCTGTGTCCATTTGGAGAGGT
GTTCAATGCCACCAGGTTTGCCTCTGTCTATGCCTGGAACAGGAAGAGGATTAGCAACTGTGTGGCTG
ACTACTCTGTGCTCTACAACTCTGCCTCCTTCAGCACCTTCAAGTGTTATGGAGTGAGCCCAACCAAA
CTGAATGACCTGTGTTTCACCAATGTCTATGCTGACTCCTTTGTGATTAGGGGAGATGAGGTGAGACA
GATTGCCCCTGGACAAACAGGCAAGATTGCTGACTACAACTACAAACTGCCTGATGACTTCACAGGCT
GTGTGATTGCCTGGAACAGCgccAACCTGGACAGCAAGGTGGGAGGCAACgccAACTACCTCTACAGA
CTGTTCAGGAAGAGCAACCTGAAACCATTTGAGAGGGACATCAGCACAGAGATTTACCAGGCTGGCAG
CACACCATGTAATGGAGTGGAGGGCTTCAACTGTTACTTTCCACTCCAATCCTATGGCTTCgccCCAg
ctgccGGAGTGGGCTACCAACCATACAGGGTGGTGGTGCTGTCCTTTGAACTGCTCCATGCCCCTGCC
ACAGTGTGTGGACCAAAGAAGAGCACCAACCTGGTGAAGAACAAGTGTGTGAACTTCAACTTCAATGG
ACTGACAGGCACAGGAGTGCTGACAGAGAGCAACAAGAAGTTCCTGCCATTCCAACAGTTTGGCAGGG
ACATTGCTGACACCACAGATGCTGTGAGGGACCCACAGACCTTGGAGATTCTGGACATCACACCATGT
TCCTTTGGAGGAGTGTCTGTGATTACACCTGGCACCAACACCAGCAACCAGGTGGCTGTGCTCTACCA
GGATGTGAACTGTACTGAGGTGCCTGTGGCTATCCATGCTGACCAACTTACACCAACCTGGAGGGTCT
ACAGCACAGGCAGCAATGTGTTCCAGACCAGGGCTGGCTGTCTGATTGGAGCAGAGCATGTGAACAAC
TCCTATGAGTGTGACATCCCAATTGGAGCAGGCATCTGTGCCTCCTACCAGACCCAGACCAACAGCCC
AAGGAGGGCAAGGTCTGTGGCAAGCCAGAGCATCATTGCCTACACAATGAGTCTGGGAGCAGAGAACT
CTGTGGCTTACAGCAACAACAGCATTGCCATCCCAACCAACTTCACCATCTCTGTGACCACAGAGATT
CTGCCTGTGAGTATGACCAAGACCTCTGTGGACTGTACAATGTATATCTGTGGAGACAGCACAGAGTG
TAGCAACCTGCTGCTCCAATATGGCTCCTTCTGTACCCAACTTAACAGGGCTCTGACAGGCATTGCTG
TGGAACAGGACAAGAACACCCAGGAGGTGTTTGCCCAGGTGAAGCAGATTTACAAGACACCTCCAATC
AAGGACTTTGGAGGCTTCAACTTCAGCCAGATTCTGCCTGACCCAAGCAAGCCAAGCAAGAGGTCCTT
CATTGAGGACCTGCTGTTCAACAAGGTGACCCTGGCTGATGCTGGCTTCATCAAGCAATATGGAGACT
GTCTGGGAGACATTGCTGCCAGGGACCTGATTTGTGCCCAGAAGTTCAATGGACTGACAGTGCTGCCT
CCACTGCTGACAGATGAGATGATTGCCCAATACACCTCTGCCCTGCTGGCTGGCACCATCACCTCTGG
CTGGACCTTTGGAGCAGGAGCAGCCCTCCAAATCCCATTTGCTATGCAGATGGCTTACAGGTTCAATG
GCATTGGAGTGACCCAGAATGTGCTCTATGAGAACCAGAAACTGATTGCCAACCAGTTCAACTCTGCC
ATTGGCAAGATTCAGGACTCCCTGTCCAGCACAGCCTCTGCCCTGGGCAAACTCCAAGATGTGGTGAA
CCAGAATGCCCAGGCTCTGAACACCCTGGTGAAGCAACTTTCCAGCAACTTTGGAGCCATCTCCTCTG
TGCTGAATGACATCCTGAGCAGACTGGACcctcccGAGGCTGAGGTCCAGATTGACAGACTGATTACA
GGCAGACTCCAATCCCTCCAAACCTATGTGACCCAACAACTTATCAGGGCTGCTGAGATTAGGGCATC
TGCCAACCTGGCTGCCACCAAGATGAGTGAGTGTGTGCTGGGACAAAGCAAGAGGGTGGACTTCTGTG
GCAAGGGCTACCACCTGATGAGTTTTCCACAGTCTGCCCCTCATGGAGTGGTGTTCCTGCATGTGACC
TATGTGCCTGCCCAGGAGAAGAACTTCACCACAGCCCCTGCCATCTGCCATGATGGCAAGGCTCACTT
TCCAAGGGAGGGAGTGTTTGTGAGCAATGGCACCCACTGGTTTGTGACCCAGAGGAACTTCTATGAAC
CACAGATTATCACCACAGACAACACCTTTGTGTCTGGCAACTGTGATGTGGTGATTGGCATTGTGAAC
AACACAGTCTATGACCCACTCCAACCTGAACTGGACTCCTTCAAGGAGGAACTGGACAAATACTTCAA
GAACCACACCAGCCCTGATGTGGACCTGGGAGACATCTCTGGCATCAATGCCTCTGTGGTGAACATCC
AGAAGGAGATTGACAGACTGAATGAGGTGGCTAAGAACCTGAATGAGTCCCTGATTGACCTCCAAGAA
CTGGGCAAAggcggaggcagcgggTATATTCCGGAGGCTCCAAGAGACGGTCAGGCGTACGTACGGAA
GGACGGCGAATGGGTTCTCTTGAGCACTTTCTTAggcCATCATCATCACCACCATATGAAGGACAACA
CAGTCCCGCTGAAACTTATTGCGCTTCTCGCAAATGGAGAGTTCCACTCTGGAGAGCAACTCGGCGAG
ACCCTGGGAATGAGTCGGGCGGCTATCAACAAGCATATACAGACTTTGAGGGATTGGGGTGTGGATGT
CTTCACTGTTCCGGGTAAGGGCTATTCACTGCCCGAACCGATCCAACTTCTCAATGCCAAACAGATAT
TGGGGCAACTTGATGGTGGCTCTGTCGCCGTGTTGCCGGTCATCGATAGCACGAACCAATATCTCTTG
GACCGCATCGGAGAACTCAAGAGCGGAGACGCATGTATAGCGGAATATCAGCAAGCCGGTAGAGGAAG
AAGGGGACGGAAATGGTTCTCTCCATTCGGAGCCAACCTGTATTTGAGCATGTTCTGGCGATTGGAGC
AGGGACCAGCCGCCGCCATCGGTCTCTCTCTTGTCATAGGTATTGTCATGGCGGAAGTCCTCCGCAAA
CTCGGGGCCGACAAAGTACGCGTGAAATGGCCGAACGACCTCTATCTTCAGGATAGGAAATTGGCAGG
GATATTGGTTGAACTGACGGGTAAGACGGGGGACGCCGCTCAGATTGTTATAGGTGCCGGAATAAACA
TGGCCATGCGCAGAGTTGAAGAGTCAGTGGTAAACCAGGGTTGGATCACTCTCCAAGAGGCGGGAATA
AACCTGGATCGCAACACACTGGCTGCCATGCTTATACGGGAACTCCGAGCTGCATTGGAACTGTTCGA
GCAAGAAGGGCTTGCTCCGTACTTGAGCAGGTGGGAAAAGCTGGATAATTTCATAAATCGCCCTGTTA
AACTCATCATCGGAGATAAAGAGATTTTTGGCATAAGTCGAGGAATCGATAAACAAGGTGCATTGCTG
CTTGAGCAAGACGGTATAATCAAACCTTGGATGGGTGGCGAAATCTCACTTAGAAGCGCGGAAAAATA
TGAACAAggcagtggagagggcagaggaagtctgctaacatgcggtgacgtcgaggagaatcctggcc
caatgagtaaaggagaagctgtgattaaagagttcatgcgcttcaaagttcacatggagggttctatg
aacggtcacgagttcgagatcgaaggcgaaggcgagggccgtccgtatgaaggcacccagaccgccaa
actgaaagtgactaaaggcggcccgctgcctttttcctgggacatcctgagcccgcaatttatgtacg
gttctagggcgttcaccaaacacccagcggatatcccggactattataagcagtcttttccggaaggt
ttcaagtgggaacgcgtaatgaattttgaagatggtggtgccgtgaccgtcactcaggacacctccct
ggaggatggcaccctgatctataaagttaaactgcgtggtactaattttccacctgatggcccggtga
tgcagaaaaagacgatgggttgggaggcgtctaccgaacgcttgtatccggaagatggtgtgctgaaa
ggcgacattaaaatggccctgcgcctgaaagatggcggccgctatctggctgacttcaaaaccacgta
caaagccaagaaacctgtgcagatgcctggcgcgtacaatgtggaccgcaaactggacatcacctctc
ataatgaagattatacggtggtagagcaatatgagcgctccgagggtcgtcattctaccggtggcatg
gatgaactatacaaataagtttaaacccgctgatcagcctcgactgtgccttctagttgccagccatc
tgttgtttgcccctcccccgtgccttccttgaccctggaaggtgccactcccactgtcctttcctaat
aaaatgaggaaattgcatcgcattgtctgagtaggtgtcattctattctggggggtggggtggggcag
gacagcaagggggaggattgggaagacaatagcaggcatgctggggatgcggtgggctctatggcttc
tgaggcggaaagaaccagctggggctctagggggtatccccacgcgccctgtagcggcgcattaagcg
cggcgggtgtggtggttacgcgcagcgtgaccgctacacttgccagcgccctagcgcccgctcctttc
gctttcttcccttcctttctcgccacgttcgccggctttccccgtcaagctctaaatcgggggctccc
tttagggttccgatttagtgctttacggcacctcgaccccaaaaaacttgattagggtgatggttcac
gtagtgggccatcgccctgatagacggtttttcgccctttgacgttggagtccacgttctttaatagt
ggactcttgttccaaactggaacaacactcaaccctatctcggtctattcttttgatttataagggat
tttgccgatttcggcctattggttaaaaaatgagctgatttaacaaaaatttaacgcgaattaattct
gtggaatgtgtgtcagttagggtgtggaaagtccccaggctccccagcaggcagaagtatgcaaagca
tgcatctcaattagtcagcaaccaggtgtggaaagtccccaggctccccagcaggcagaagtatgcaa
agcatgcatctcaattagtcagcaaccatagtcccgcccctaactccgcccatcccgcccctaactcc
gcccagttccgcccattctccgccccatggctgactaattttttttatttatgcagaggccgaggccg
cctctgcctctgagctattccagaagtagtgaggaggcttttttggaggcctaggcttttgcaaaaag
ctcccgggagcttgtatatccattttcggatctgatcaagagacaggatgaggatcgtttcgcatgat
tgaacaagatggattgcacgcaggttctccggccgcttgggtggagaggctattcggctatgactggg
cacaacagacaatcggctgctctgatgccgccgtgttccggctgtcagcgcaggggcgcccggttctt
tttgtcaagaccgacctgtccggtgccctgaatgaactgcaggacgaggcagcgcggctatcgtggct
ggccacgacgggcgttccttgcgcagctgtgctcgacgttgtcactgaagcgggaagggactggctgc
tattgggcgaagtgccggggcaggatctcctgtcatctcaccttgctcctgccgagaaagtatccatc
atggctgatgcaatgcggcggctgcatacgcttgatccggctacctgcccattcgaccaccaagcgaa
acatcgcatcgagcgagcacgtactcggatggaagccggtcttgtcgatcaggatgatctggacgaag
agcatcaggggctcgcgccagccgaactgttcgccaggctcaaggcgcgcatgcccgacggcgaggat
ctcgtcgtgacccatggcgatgcctgcttgccgaatatcatggtggaaaatggccgcttttctggatt
catcgactgtggccggctgggtgtggcggaccgctatcaggacatagcgttggctacccgtgatattg
ctgaagagcttggcggcgaatgggctgaccgcttcctcgtgctttacggtatcgccgctcccgattcg
cagcgcatcgccttctatcgccttcttgacgagttcttctgagcgggactctggggttcgaaatgacc
gaccaagcgacgcccaacctgccatcacgagatttcgattccaccgccgccttctatgaaaggttggg
cttcggaatcgttttccgggacgccggctggatgatcctccagcgcggggatctcatgctggagttct
tcgcccaccccaacttgtttattgcagcttataatggttacaaataaagcaatagcatcacaaatttc
acaaataaagcatttttttcactgcattctagttgtggtttgtccaaactcatcaatgtatcttatca
tgtctgtataccgtcgacctctagctagagcttggcgtaatcatggtcatagctgtttcctgtgtgaa
attgttatccgctcacaattccacacaacatacgagccggaagcataaagtgtaaagcctggggtgcc
taatgagtgagctaactcacattaattgcgttgcgctcactgcccgctttccagtcgggaaacctgtc
gtgccagctgcattaatgaatcggccaacgcgcggggagaggcggtttgcgtattgggcgctcttccg
cttcctcgctcactgactcgctgcgctcggtcgttcggctgcggcgagcggtatcagctcactcaaag
gcggtaatacggttatccacagaatcaggggataacgcaggaaagaacatgtgagcaaaaggccagca
aaaggccaggaaccgtaaaaaggccgcgttgctggcgtttttccataggctccgcccccctgacgagc
atcacaaaaatcgacgctcaagtcagaggtggcgaaacccgacaggactataaagataccaggcgttt
ccccctggaagctccctcgtgcgctctcctgttccgaccctgccgcttaccggatacctgtccgcctt
tctcccttcgggaagcgtggcgctttctcatagctcacgctgtaggtatctcagttcggtgtaggtcg
ttcgctccaagctgggctgtgtgcacgaaccccccgttcagcccgaccgctgcgccttatccggtaac
tatcgtcttgagtccaacccggtaagacacgacttatcgccactggcagcagccactggtaacaggat
tagcagagcgaggtatgtaggcggtgctacagagttcttgaagtggtggcctaactacggctacacta
gaagaacagtatttggtatctgcgctctgctgaagccagttaccttcggaaaaagagttggtagctct
tgatccggcaaacaaaccaccgctggtagcggtttttttgtttgcaagcagcagattacgcgcagaaa
aaaaggatctcaagaagatcctttgatcttttctacggggtctgacgctcagtggaacgaaaactcac
gttaagggattttggtcatgagattatcaaaaaggatcttcacctagatccttttaaattaaaaatga
agttttaaatcaatctaaagtatatatgagtaaacttggtctgacagttaccaatgcttaatcagtga
ggcacctatctcagcgatctgtctatttcgttcatccatagttgcctgactccccgtcgtgtagataa
ctacgatacgggagggcttaccatctggccccagtgctgcaatgataccgcgagacccacgctcaccg
gctccagatttatcagcaataaaccagccagccggaagggccgagcgcagaagtggtcctgcaacttt
atccgcctccatccagtctattaattgttgccgggaagctagagtaagtagttcgccagttaatagtt
tgcgcaacgttgttgccattgctacaggcatcgtggtgtcacgctcgtcgtttggtatggcttcattc
agctccggttcccaacgatcaaggcgagttacatgatcccccatgttgtgcaaaaaagcggttagctc
cttcggtcctccgatcgttgtcagaagtaagttggccgcagtgttatcactcatggttatggcagcac
tgcataattctcttactgtcatgccatccgtaagatgcttttctgtgactggtgagtactcaaccaag
tcattctgagaatagtgtatgcggcgaccgagttgctcttgcccggcgtcaatacgggataataccgc
gccacatagcagaactttaaaagtgctcatcattggaaaacgttcttcggggcgaaaactctcaagga
tcttaccgctgttgagatccagttcgatgtaacccactcgtgcacccaactgatcttcagcatctttt
actttcaccagcgtttctgggtgagcaaaaacaggaaggcaaaatgccgcaaaaaagggaataagggc
gacacggaaatgttgaatactcatactcttcctttttcaatattattgaagcatttatcagggttatt
gtctcatgagcggatacatatttgaatgtatttagaaaaataaacaaataggggttccgcgcacattt
ccccgaaaagtgccacctgacgtc
pMD139-pcDNA3.1-SARS2_mut_v2-GGSG-foldon-G-6xHis-BirA-T2A-mScarlet
SEQ ID NO: 116
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGAGGGCAAG
CAGGGCAACTTCAAGAACCTGAGGGAGTTTGTGTTCAAGAACATTGATGGCTACTTCAAGATTTACAG
CAAACACACACCAATCAACCTGGTGAGGGACCTGCCACAGGGCTTCTCTGCCTTGGAACCACTGGTGG
ACCTGCCAATTGGCATCAACATCACCAGGTTCCAGACCCTGCTGGCTCTGCACAGGTCCTACCTGACA
CCTGGAGACTCCTCCTCTGGCTGGACAGCAGGAGCAGCAGCCTACTATGTGGGCTACCTCCAACCAAG
GACCTTCCTGCTGAAATACAATGAGAATGGCACCATCACAGATGCTGTGGACTGTGCCCTGGACCCAC
TGTCTGAGACCAAGTGTACCCTGAAATCCTTCACAGTGGAGAAGGGCATCTACCAGACCAGCAACTTC
AGGGTCCAACCAACAGAGAGCATTGTGAGGTTTCCAAACATCACCAACCTGTGTCCATTTGGAGAGGT
GTTCAATGCCACCAGGTTTGCCTCTGTCTATGCCTGGAACAGGAAGAGGATTAGCAACTGTGTGGCTG
ACTACTCTGTGCTCTACAACTCTGCCTCCTTCAGCACCTTCAAGTGTTATGGAGTGAGCCCAACCAAA
CTGAATGACCTGTGTTTCACCAATGTCTATGCTGACTCCTTTGTGATTAGGGGAGATGAGGTGAGACA
GATTGCCCCTGGACAAACAGGCgccATTGCTGACTACAACTACAAACTGCCTGATGACTTCACAGGCT
GTGTGATTGCCTGGAACAGCAACAACCTGGACAGCAAGGTGGGAGGCAACTACAACTACCTCTACAGA
gccgctAGGAAGAGCAACCTGAAACCATTTGAGAGGGACATCAGCACAGAGATTgccCAGGCTGGCAG
CACACCATGTAATGGAGTGGAGGGCTTCAACTGTTACTTTCCACTCgccTCCTATGGCTTCCAACCAA
CCAATGGAGTGGGCTACCAACCATACAGGGTGGTGGTGCTGTCCTTTGAACTGCTCCATGCCCCTGCC
ACAGTGTGTGGACCAAAGAAGAGCACCAACCTGGTGAAGAACAAGTGTGTGAACTTCAACgccAATGG
ACTGACAGGCACAGGAGTGCTGACAGAGAGCAACAAGAAGTTCCTGCCATTCCAACAGTTTGGCAGGG
ACATTGCTGACACCACAGATGCTGTGAGGGACCCACAGACCTTGGAGATTCTGGACATCACACCATGT
TCCTTTGGAGGAGTGTCTGTGATTACACCTGGCACCAACACCAGCAACCAGGTGGCTGTGCTCTACCA
GGATGTGAACTGTACTGAGGTGCCTGTGGCTATCCATGCTGACCAACTTACACCAACCTGGAGGGTCT
ACAGCACAGGCAGCAATGTGTTCCAGACCAGGGCTGGCTGTCTGATTGGAGCAGAGCATGTGAACAAC
TCCTATGAGTGTGACATCCCAATTGGAGCAGGCATCTGTGCCTCCTACCAGACCCAGACCAACAGCCC
AAGGAGGGCAAGGTCTGTGGCAAGCCAGAGCATCATTGCCTACACAATGAGTCTGGGAGCAGAGAACT
CTGTGGCTTACAGCAACAACAGCATTGCCATCCCAACCAACTTCACCATCTCTGTGACCACAGAGATT
CTGCCTGTGAGTATGACCAAGACCTCTGTGGACTGTACAATGTATATCTGTGGAGACAGCACAGAGTG
TAGCAACCTGCTGCTCCAATATGGCTCCTTCTGTACCCAACTTAACAGGGCTCTGACAGGCATTGCTG
TGGAACAGGACAAGAACACCCAGGAGGTGTTTGCCCAGGTGAAGCAGATTTACAAGACACCTCCAATC
AAGGACTTTGGAGGCTTCAACTTCAGCCAGATTCTGCCTGACCCAAGCAAGCCAAGCAAGAGGTCCTT
CATTGAGGACCTGCTGTTCAACAAGGTGACCCTGGCTGATGCTGGCTTCATCAAGCAATATGGAGACT
GTCTGGGAGACATTGCTGCCAGGGACCTGATTTGTGCCCAGAAGTTCAATGGACTGACAGTGCTGCCT
CCACTGCTGACAGATGAGATGATTGCCCAATACACCTCTGCCCTGCTGGCTGGCACCATCACCTCTGG
CTGGACCTTTGGAGCAGGAGCAGCCCTCCAAATCCCATTTGCTATGCAGATGGCTTACAGGTTCAATG
GCATTGGAGTGACCCAGAATGTGCTCTATGAGAACCAGAAACTGATTGCCAACCAGTTCAACTCTGCC
ATTGGCAAGATTCAGGACTCCCTGTCCAGCACAGCCTCTGCCCTGGGCAAACTCCAAGATGTGGTGAA
CCAGAATGCCCAGGCTCTGAACACCCTGGTGAAGCAACTTTCCAGCAACTTTGGAGCCATCTCCTCTG
TGCTGAATGACATCCTGAGCAGACTGGACcctcccGAGGCTGAGGTCCAGATTGACAGACTGATTACA
GGCAGACTCCAATCCCTCCAAACCTATGTGACCCAACAACTTATCAGGGCTGCTGAGATTAGGGCATC
TGCCAACCTGGCTGCCACCAAGATGAGTGAGTGTGTGCTGGGACAAAGCAAGAGGGTGGACTTCTGTG
GCAAGGGCTACCACCTGATGAGTTTTCCACAGTCTGCCCCTCATGGAGTGGTGTTCCTGCATGTGACC
TATGTGCCTGCCCAGGAGAAGAACTTCACCACAGCCCCTGCCATCTGCCATGATGGCAAGGCTCACTT
TCCAAGGGAGGGAGTGTTTGTGAGCAATGGCACCCACTGGTTTGTGACCCAGAGGAACTTCTATGAAC
CACAGATTATCACCACAGACAACACCTTTGTGTCTGGCAACTGTGATGTGGTGATTGGCATTGTGAAC
AACACAGTCTATGACCCACTCCAACCTGAACTGGACTCCTTCAAGGAGGAACTGGACAAATACTTCAA
GAACCACACCAGCCCTGATGTGGACCTGGGAGACATCTCTGGCATCAATGCCTCTGTGGTGAACATCC
AGAAGGAGATTGACAGACTGAATGAGGTGGCTAAGAACCTGAATGAGTCCCTGATTGACCTCCAAGAA
CTGGGCAAAggcggaggcagcgggTATATTCCGGAGGCTCCAAGAGACGGTCAGGCGTACGTACGGAA
GGACGGCGAATGGGTTCTCTTGAGCACTTTCTTAggcCATCATCATCACCACCATATGAAGGACAACA
CAGTCCCGCTGAAACTTATTGCGCTTCTCGCAAATGGAGAGTTCCACTCTGGAGAGCAACTCGGCGAG
ACCCTGGGAATGAGTCGGGCGGCTATCAACAAGCATATACAGACTTTGAGGGATTGGGGTGTGGATGT
CTTCACTGTTCCGGGTAAGGGCTATTCACTGCCCGAACCGATCCAACTTCTCAATGCCAAACAGATAT
TGGGGCAACTTGATGGTGGCTCTGTCGCCGTGTTGCCGGTCATCGATAGCACGAACCAATATCTCTTG
GACCGCATCGGAGAACTCAAGAGCGGAGACGCATGTATAGCGGAATATCAGCAAGCCGGTAGAGGAAG
AAGGGGACGGAAATGGTTCTCTCCATTCGGAGCCAACCTGTATTTGAGCATGTTCTGGCGATTGGAGC
AGGGACCAGCCGCCGCCATCGGTCTCTCTCTTGTCATAGGTATTGTCATGGCGGAAGTCCTCCGCAAA
CTCGGGGCCGACAAAGTACGCGTGAAATGGCCGAACGACCTCTATCTTCAGGATAGGAAATTGGCAGG
GATATTGGTTGAACTGACGGGTAAGACGGGGGACGCCGCTCAGATTGTTATAGGTGCCGGAATAAACA
TGGCCATGCGCAGAGTTGAAGAGTCAGTGGTAAACCAGGGTTGGATCACTCTCCAAGAGGCGGGAATA
AACCTGGATCGCAACACACTGGCTGCCATGCTTATACGGGAACTCCGAGCTGCATTGGAACTGTTCGA
GCAAGAAGGGCTTGCTCCGTACTTGAGCAGGTGGGAAAAGCTGGATAATTTCATAAATCGCCCTGTTA
AACTCATCATCGGAGATAAAGAGATTTTTGGCATAAGTCGAGGAATCGATAAACAAGGTGCATTGCTG
CTTGAGCAAGACGGTATAATCAAACCTTGGATGGGTGGCGAAATCTCACTTAGAAGCGCGGAAAAATA
TGAACAAggcagtggagagggcagaggaagtctgctaacatgcggtgacgtcgaggagaatcctggcc
caatgagtaaaggagaagctgtgattaaagagttcatgcgcttcaaagttcacatggagggttctatg
aacggtcacgagttcgagatcgaaggcgaaggcgagggccgtccgtatgaaggcacccagaccgccaa
actgaaagtgactaaaggcggcccgctgcctttttcctgggacatcctgagcccgcaatttatgtacg
gttctagggcgttcaccaaacacccagcggatatcccggactattataagcagtcttttccggaaggt
ttcaagtgggaacgcgtaatgaattttgaagatggtggtgccgtgaccgtcactcaggacacctccct
ggaggatggcaccctgatctataaagttaaactgcgtggtactaattttccacctgatggcccggtga
tgcagaaaaagacgatgggttgggaggcgtctaccgaacgcttgtatccggaagatggtgtgctgaaa
ggcgacattaaaatggccctgcgcctgaaagatggcggccgctatctggctgacttcaaaaccacgta
caaagccaagaaacctgtgcagatgcctggcgcgtacaatgtggaccgcaaactggacatcacctctc
ataatgaagattatacggtggtagagcaatatgagcgctccgagggtcgtcattctaccggtggcatg
gatgaactatacaaataagtttaaacccgctgatcagcctcgactgtgccttctagttgccagccatc
tgttgtttgcccctcccccgtgccttccttgaccctggaaggtgccactcccactgtcctttcctaat
aaaatgaggaaattgcatcgcattgtctgagtaggtgtcattctattctggggggtggggtggggcag
gacagcaagggggaggattgggaagacaatagcaggcatgctggggatgcggtgggctctatggcttc
tgaggcggaaagaaccagctggggctctagggggtatccccacgcgccctgtagcggcgcattaagcg
cggcgggtgtggtggttacgcgcagcgtgaccgctacacttgccagcgccctagcgcccgctcctttc
gctttcttcccttcctttctcgccacgttcgccggctttccccgtcaagctctaaatcgggggctccc
tttagggttccgatttagtgctttacggcacctcgaccccaaaaaacttgattagggtgatggttcac
gtagtgggccatcgccctgatagacggtttttcgccctttgacgttggagtccacgttctttaatagt
ggactcttgttccaaactggaacaacactcaaccctatctcggtctattcttttgatttataagggat
tttgccgatttcggcctattggttaaaaaatgagctgatttaacaaaaatttaacgcgaattaattct
gtggaatgtgtgtcagttagggtgtggaaagtccccaggctccccagcaggcagaagtatgcaaagca
tgcatctcaattagtcagcaaccaggtgtggaaagtccccaggctccccagcaggcagaagtatgcaa
agcatgcatctcaattagtcagcaaccatagtcccgcccctaactccgcccatcccgcccctaactcc
gcccagttccgcccattctccgccccatggctgactaattttttttatttatgcagaggccgaggccg
cctctgcctctgagctattccagaagtagtgaggaggcttttttggaggcctaggcttttgcaaaaag
ctcccgggagcttgtatatccattttcggatctgatcaagagacaggatgaggatcgtttcgcatgat
tgaacaagatggattgcacgcaggttctccggccgcttgggtggagaggctattcggctatgactggg
cacaacagacaatcggctgctctgatgccgccgtgttccggctgtcagcgcaggggcgcccggttctt
tttgtcaagaccgacctgtccggtgccctgaatgaactgcaggacgaggcagcgcggctatcgtggct
ggccacgacgggcgttccttgcgcagctgtgctcgacgttgtcactgaagcgggaagggactggctgc
tattgggcgaagtgccggggcaggatctcctgtcatctcaccttgctcctgccgagaaagtatccatc
atggctgatgcaatgcggcggctgcatacgcttgatccggctacctgcccattcgaccaccaagcgaa
acatcgcatcgagcgagcacgtactcggatggaagccggtcttgtcgatcaggatgatctggacgaag
agcatcaggggctcgcgccagccgaactgttcgccaggctcaaggcgcgcatgcccgacggcgaggat
ctcgtcgtgacccatggcgatgcctgcttgccgaatatcatggtggaaaatggccgcttttctggatt
catcgactgtggccggctgggtgtggcggaccgctatcaggacatagcgttggctacccgtgatattg
ctgaagagcttggcggcgaatgggctgaccgcttcctcgtgctttacggtatcgccgctcccgattcg
cagcgcatcgccttctatcgccttcttgacgagttcttctgagcgggactctggggttcgaaatgacc
gaccaagcgacgcccaacctgccatcacgagatttcgattccaccgccgccttctatgaaaggttggg
cttcggaatcgttttccgggacgccggctggatgatcctccagcgcggggatctcatgctggagttct
tcgcccaccccaacttgtttattgcagcttataatggttacaaataaagcaatagcatcacaaatttc
acaaataaagcatttttttcactgcattctagttgtggtttgtccaaactcatcaatgtatcttatca
tgtctgtataccgtcgacctctagctagagcttggcgtaatcatggtcatagctgtttcctgtgtgaa
attgttatccgctcacaattccacacaacatacgagccggaagcataaagtgtaaagcctggggtgcc
taatgagtgagctaactcacattaattgcgttgcgctcactgcccgctttccagtcgggaaacctgtc
gtgccagctgcattaatgaatcggccaacgcgcggggagaggcggtttgcgtattgggcgctcttccg
cttcctcgctcactgactcgctgcgctcggtcgttcggctgcggcgagcggtatcagctcactcaaag
gcggtaatacggttatccacagaatcaggggataacgcaggaaagaacatgtgagcaaaaggccagca
aaaggccaggaaccgtaaaaaggccgcgttgctggcgtttttccataggctccgcccccctgacgagc
atcacaaaaatcgacgctcaagtcagaggtggcgaaacccgacaggactataaagataccaggcgttt
ccccctggaagctccctcgtgcgctctcctgttccgaccctgccgcttaccggatacctgtccgcctt
tctcccttcgggaagcgtggcgctttctcatagctcacgctgtaggtatctcagttcggtgtaggtcg
ttcgctccaagctgggctgtgtgcacgaaccccccgttcagcccgaccgctgcgccttatccggtaac
tatcgtcttgagtccaacccggtaagacacgacttatcgccactggcagcagccactggtaacaggat
tagcagagcgaggtatgtaggcggtgctacagagttcttgaagtggtggcctaactacggctacacta
gaagaacagtatttggtatctgcgctctgctgaagccagttaccttcggaaaaagagttggtagctct
tgatccggcaaacaaaccaccgctggtagcggtttttttgtttgcaagcagcagattacgcgcagaaa
aaaaggatctcaagaagatcctttgatcttttctacggggtctgacgctcagtggaacgaaaactcac
gttaagggattttggtcatgagattatcaaaaaggatcttcacctagatccttttaaattaaaaatga
agttttaaatcaatctaaagtatatatgagtaaacttggtctgacagttaccaatgcttaatcagtga
ggcacctatctcagcgatctgtctatttcgttcatccatagttgcctgactccccgtcgtgtagataa
ctacgatacgggagggcttaccatctggccccagtgctgcaatgataccgcgagacccacgctcaccg
gctccagatttatcagcaataaaccagccagccggaagggccgagcgcagaagtggtcctgcaacttt
atccgcctccatccagtctattaattgttgccgggaagctagagtaagtagttcgccagttaatagtt
tgcgcaacgttgttgccattgctacaggcatcgtggtgtcacgctcgtcgtttggtatggcttcattc
agctccggttcccaacgatcaaggcgagttacatgatcccccatgttgtgcaaaaaagcggttagctc
cttcggtcctccgatcgttgtcagaagtaagttggccgcagtgttatcactcatggttatggcagcac
tgcataattctcttactgtcatgccatccgtaagatgcttttctgtgactggtgagtactcaaccaag
tcattctgagaatagtgtatgcggcgaccgagttgctcttgcccggcgtcaatacgggataataccgc
gccacatagcagaactttaaaagtgctcatcattggaaaacgttcttcggggcgaaaactctcaagga
tcttaccgctgttgagatccagttcgatgtaacccactcgtgcacccaactgatcttcagcatctttt
actttcaccagcgtttctgggtgagcaaaaacaggaaggcaaaatgccgcaaaaaagggaataagggc
gacacggaaatgttgaatactcatactcttcctttttcaatattattgaagcatttatcagggttatt
gtctcatgagcggatacatatttgaatgtatttagaaaaataaacaaataggggttccgcgcacattt
ccccgaaaagtgccacctgacgtc
pMD140-pcDNA3.1-SARS2_mut_v3-GGSG-foldon-G-6xHis-BirA-T2A-mScarlet
SEQ ID NO: 117
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGAGGGCAAG
CAGGGCAACTTCAAGAACCTGAGGGAGTTTGTGTTCAAGAACATTGATGGCTACTTCAAGATTTACAG
CAAACACACACCAATCAACCTGGTGAGGGACCTGCCACAGGGCTTCTCTGCCTTGGAACCACTGGTGG
ACCTGCCAATTGGCATCAACATCACCAGGTTCCAGACCCTGCTGGCTCTGCACAGGTCCTACCTGACA
CCTGGAGACTCCTCCTCTGGCTGGACAGCAGGAGCAGCAGCCTACTATGTGGGCTACCTCCAACCAAG
GACCTTCCTGCTGAAATACAATGAGAATGGCACCATCACAGATGCTGTGGACTGTGCCCTGGACCCAC
TGTCTGAGACCAAGTGTACCCTGAAATCCTTCACAGTGGAGAAGGGCATCTACCAGACCAGCAACTTC
AGGGTCCAACCAACAGAGAGCATTGTGAGGTTTCCAAACATCACCAACCTGTGTCCATTTGGAGAGGT
GTTCAATGCCACCAGGTTTGCCTCTGTCTATGCCTGGAACAGGAAGAGGATTAGCAACTGTGTGGCTG
ACTACTCTGTGCTCTACAACTCTGCCTCCTTCAGCACCTTCAAGTGTTATGGAGTGAGCCCAACCAAA
CTGAATGACCTGTGTTTCACCAATGTCTATGCTGACTCCTTTGTGATTAGGGGAGATGAGGTGAGACA
GATTGCCCCTGGACAAACAGGCAAGATTGCTGACTACAACTACAAACTGCCTGATGACTTCACAGGCT
GTGTGATTGCCTGGAACAGCAACAACCTGGACAGCAAGGTGGGAGGCAACTACAACTACCTCTACAGA
CTGTTCAGGAAGAGCAACCTGAAACCATTTGAGAGGGACATCAGCACAGAGATTTACgctGCTGGCAG
CACACCATGTAATGGAGTGGAGGGCgccgctTGTTACTTTCCACTCCAATCCTATGGCTTCCAACCAA
CCAATGGAGTGGGCTACCAACCATACAGGGTGGTGGTGCTGTCCTTTGAACTGCTCCATGCCCCTGCC
ACAGTGTGTGGACCAAAGAAGAGCACCAACCTGGTGAAGAACAAGTGTGTGAACTTCAACTTCAATGG
ACTGACAGGCACAGGAGTGCTGACAGAGAGCAACAAGAAGTTCCTGCCATTCCAACAGTTTGGCAGGG
ACATTGCTGACACCACAGATGCTGTGAGGGACCCACAGACCTTGGAGATTCTGGACATCACACCATGT
TCCTTTGGAGGAGTGTCTGTGATTACACCTGGCACCAACACCAGCAACCAGGTGGCTGTGCTCTACCA
GGATGTGAACTGTACTGAGGTGCCTGTGGCTATCCATGCTGACCAACTTACACCAACCTGGAGGGTCT
ACAGCACAGGCAGCAATGTGTTCCAGACCAGGGCTGGCTGTCTGATTGGAGCAGAGCATGTGAACAAC
TCCTATGAGTGTGACATCCCAATTGGAGCAGGCATCTGTGCCTCCTACCAGACCCAGACCAACAGCCC
AAGGAGGGCAAGGTCTGTGGCAAGCCAGAGCATCATTGCCTACACAATGAGTCTGGGAGCAGAGAACT
CTGTGGCTTACAGCAACAACAGCATTGCCATCCCAACCAACTTCACCATCTCTGTGACCACAGAGATT
CTGCCTGTGAGTATGACCAAGACCTCTGTGGACTGTACAATGTATATCTGTGGAGACAGCACAGAGTG
TAGCAACCTGCTGCTCCAATATGGCTCCTTCTGTACCCAACTTAACAGGGCTCTGACAGGCATTGCTG
TGGAACAGGACAAGAACACCCAGGAGGTGTTTGCCCAGGTGAAGCAGATTTACAAGACACCTCCAATC
AAGGACTTTGGAGGCTTCAACTTCAGCCAGATTCTGCCTGACCCAAGCAAGCCAAGCAAGAGGTCCTT
CATTGAGGACCTGCTGTTCAACAAGGTGACCCTGGCTGATGCTGGCTTCATCAAGCAATATGGAGACT
GTCTGGGAGACATTGCTGCCAGGGACCTGATTTGTGCCCAGAAGTTCAATGGACTGACAGTGCTGCCT
CCACTGCTGACAGATGAGATGATTGCCCAATACACCTCTGCCCTGCTGGCTGGCACCATCACCTCTGG
CTGGACCTTTGGAGCAGGAGCAGCCCTCCAAATCCCATTTGCTATGCAGATGGCTTACAGGTTCAATG
GCATTGGAGTGACCCAGAATGTGCTCTATGAGAACCAGAAACTGATTGCCAACCAGTTCAACTCTGCC
ATTGGCAAGATTCAGGACTCCCTGTCCAGCACAGCCTCTGCCCTGGGCAAACTCCAAGATGTGGTGAA
CCAGAATGCCCAGGCTCTGAACACCCTGGTGAAGCAACTTTCCAGCAACTTTGGAGCCATCTCCTCTG
TGCTGAATGACATCCTGAGCAGACTGGACcctcccGAGGCTGAGGTCCAGATTGACAGACTGATTACA
GGCAGACTCCAATCCCTCCAAACCTATGTGACCCAACAACTTATCAGGGCTGCTGAGATTAGGGCATC
TGCCAACCTGGCTGCCACCAAGATGAGTGAGTGTGTGCTGGGACAAAGCAAGAGGGTGGACTTCTGTG
GCAAGGGCTACCACCTGATGAGTTTTCCACAGTCTGCCCCTCATGGAGTGGTGTTCCTGCATGTGACC
TATGTGCCTGCCCAGGAGAAGAACTTCACCACAGCCCCTGCCATCTGCCATGATGGCAAGGCTCACTT
TCCAAGGGAGGGAGTGTTTGTGAGCAATGGCACCCACTGGTTTGTGACCCAGAGGAACTTCTATGAAC
CACAGATTATCACCACAGACAACACCTTTGTGTCTGGCAACTGTGATGTGGTGATTGGCATTGTGAAC
AACACAGTCTATGACCCACTCCAACCTGAACTGGACTCCTTCAAGGAGGAACTGGACAAATACTTCAA
GAACCACACCAGCCCTGATGTGGACCTGGGAGACATCTCTGGCATCAATGCCTCTGTGGTGAACATCC
AGAAGGAGATTGACAGACTGAATGAGGTGGCTAAGAACCTGAATGAGTCCCTGATTGACCTCCAAGAA
CTGGGCAAAggcggaggcagcgggTATATTCCGGAGGCTCCAAGAGACGGTCAGGCGTACGTACGGAA
GGACGGCGAATGGGTTCTCTTGAGCACTTTCTTAggcCATCATCATCACCACCATATGAAGGACAACA
CAGTCCCGCTGAAACTTATTGCGCTTCTCGCAAATGGAGAGTTCCACTCTGGAGAGCAACTCGGCGAG
ACCCTGGGAATGAGTCGGGCGGCTATCAACAAGCATATACAGACTTTGAGGGATTGGGGTGTGGATGT
CTTCACTGTTCCGGGTAAGGGCTATTCACTGCCCGAACCGATCCAACTTCTCAATGCCAAACAGATAT
TGGGGCAACTTGATGGTGGCTCTGTCGCCGTGTTGCCGGTCATCGATAGCACGAACCAATATCTCTTG
GACCGCATCGGAGAACTCAAGAGCGGAGACGCATGTATAGCGGAATATCAGCAAGCCGGTAGAGGAAG
AAGGGGACGGAAATGGTTCTCTCCATTCGGAGCCAACCTGTATTTGAGCATGTTCTGGCGATTGGAGC
AGGGACCAGCCGCCGCCATCGGTCTCTCTCTTGTCATAGGTATTGTCATGGCGGAAGTCCTCCGCAAA
CTCGGGGCCGACAAAGTACGCGTGAAATGGCCGAACGACCTCTATCTTCAGGATAGGAAATTGGCAGG
GATATTGGTTGAACTGACGGGTAAGACGGGGGACGCCGCTCAGATTGTTATAGGTGCCGGAATAAACA
TGGCCATGCGCAGAGTTGAAGAGTCAGTGGTAAACCAGGGTTGGATCACTCTCCAAGAGGCGGGAATA
AACCTGGATCGCAACACACTGGCTGCCATGCTTATACGGGAACTCCGAGCTGCATTGGAACTGTTCGA
GCAAGAAGGGCTTGCTCCGTACTTGAGCAGGTGGGAAAAGCTGGATAATTTCATAAATCGCCCTGTTA
AACTCATCATCGGAGATAAAGAGATTTTTGGCATAAGTCGAGGAATCGATAAACAAGGTGCATTGCTG
CTTGAGCAAGACGGTATAATCAAACCTTGGATGGGTGGCGAAATCTCACTTAGAAGCGCGGAAAAATA
TGAACAAggcagtggagagggcagaggaagtctgctaacatgcggtgacgtcgaggagaatcctggcc
caatgagtaaaggagaagctgtgattaaagagttcatgcgcttcaaagttcacatggagggttctatg
aacggtcacgagttcgagatcgaaggcgaaggcgagggccgtccgtatgaaggcacccagaccgccaa
actgaaagtgactaaaggcggcccgctgcctttttcctgggacatcctgagcccgcaatttatgtacg
gttctagggcgttcaccaaacacccagcggatatcccggactattataagcagtcttttccggaaggt
ttcaagtgggaacgcgtaatgaattttgaagatggtggtgccgtgaccgtcactcaggacacctccct
ggaggatggcaccctgatctataaagttaaactgcgtggtactaattttccacctgatggcccggtga
tgcagaaaaagacgatgggttgggaggcgtctaccgaacgcttgtatccggaagatggtgtgctgaaa
ggcgacattaaaatggccctgcgcctgaaagatggcggccgctatctggctgacttcaaaaccacgta
caaagccaagaaacctgtgcagatgcctggcgcgtacaatgtggaccgcaaactggacatcacctctc
ataatgaagattatacggtggtagagcaatatgagcgctccgagggtcgtcattctaccggtggcatg
gatgaactatacaaataagtttaaacccgctgatcagcctcgactgtgccttctagttgccagccatc
tgttgtttgcccctcccccgtgccttccttgaccctggaaggtgccactcccactgtcctttcctaat
aaaatgaggaaattgcatcgcattgtctgagtaggtgtcattctattctggggggtggggtggggcag
gacagcaagggggaggattgggaagacaatagcaggcatgctggggatgcggtgggctctatggcttc
tgaggcggaaagaaccagctggggctctagggggtatccccacgcgccctgtagcggcgcattaagcg
cggcgggtgtggtggttacgcgcagcgtgaccgctacacttgccagcgccctagcgcccgctcctttc
gctttcttcccttcctttctcgccacgttcgccggctttccccgtcaagctctaaatcgggggctccc
tttagggttccgatttagtgctttacggcacctcgaccccaaaaaacttgattagggtgatggttcac
gtagtgggccatcgccctgatagacggtttttcgccctttgacgttggagtccacgttctttaatagt
ggactcttgttccaaactggaacaacactcaaccctatctcggtctattcttttgatttataagggat
tttgccgatttcggcctattggttaaaaaatgagctgatttaacaaaaatttaacgcgaattaattct
gtggaatgtgtgtcagttagggtgtggaaagtccccaggctccccagcaggcagaagtatgcaaagca
tgcatctcaattagtcagcaaccaggtgtggaaagtccccaggctccccagcaggcagaagtatgcaa
agcatgcatctcaattagtcagcaaccatagtcccgcccctaactccgcccatcccgcccctaactcc
gcccagttccgcccattctccgccccatggctgactaattttttttatttatgcagaggccgaggccg
cctctgcctctgagctattccagaagtagtgaggaggcttttttggaggcctaggcttttgcaaaaag
ctcccgggagcttgtatatccattttcggatctgatcaagagacaggatgaggatcgtttcgcatgat
tgaacaagatggattgcacgcaggttctccggccgcttgggtggagaggctattcggctatgactggg
cacaacagacaatcggctgctctgatgccgccgtgttccggctgtcagcgcaggggcgcccggttctt
tttgtcaagaccgacctgtccggtgccctgaatgaactgcaggacgaggcagcgcggctatcgtggct
ggccacgacgggcgttccttgcgcagctgtgctcgacgttgtcactgaagcgggaagggactggctgc
tattgggcgaagtgccggggcaggatctcctgtcatctcaccttgctcctgccgagaaagtatccatc
atggctgatgcaatgcggcggctgcatacgcttgatccggctacctgcccattcgaccaccaagcgaa
acatcgcatcgagcgagcacgtactcggatggaagccggtcttgtcgatcaggatgatctggacgaag
agcatcaggggctcgcgccagccgaactgttcgccaggctcaaggcgcgcatgcccgacggcgaggat
ctcgtcgtgacccatggcgatgcctgcttgccgaatatcatggtggaaaatggccgcttttctggatt
catcgactgtggccggctgggtgtggcggaccgctatcaggacatagcgttggctacccgtgatattg
ctgaagagcttggcggcgaatgggctgaccgcttcctcgtgctttacggtatcgccgctcccgattcg
cagcgcatcgccttctatcgccttcttgacgagttcttctgagcgggactctggggttcgaaatgacc
gaccaagcgacgcccaacctgccatcacgagatttcgattccaccgccgccttctatgaaaggttggg
cttcggaatcgttttccgggacgccggctggatgatcctccagcgcggggatctcatgctggagttct
tcgcccaccccaacttgtttattgcagcttataatggttacaaataaagcaatagcatcacaaatttc
acaaataaagcatttttttcactgcattctagttgtggtttgtccaaactcatcaatgtatcttatca
tgtctgtataccgtcgacctctagctagagcttggcgtaatcatggtcatagctgtttcctgtgtgaa
attgttatccgctcacaattccacacaacatacgagccggaagcataaagtgtaaagcctggggtgcc
taatgagtgagctaactcacattaattgcgttgcgctcactgcccgctttccagtcgggaaacctgtc
gtgccagctgcattaatgaatcggccaacgcgcggggagaggcggtttgcgtattgggcgctcttccg
cttcctcgctcactgactcgctgcgctcggtcgttcggctgcggcgagcggtatcagctcactcaaag
gcggtaatacggttatccacagaatcaggggataacgcaggaaagaacatgtgagcaaaaggccagca
aaaggccaggaaccgtaaaaaggccgcgttgctggcgtttttccataggctccgcccccctgacgagc
atcacaaaaatcgacgctcaagtcagaggtggcgaaacccgacaggactataaagataccaggcgttt
ccccctggaagctccctcgtgcgctctcctgttccgaccctgccgcttaccggatacctgtccgcctt
tctcccttcgggaagcgtggcgctttctcatagctcacgctgtaggtatctcagttcggtgtaggtcg
ttcgctccaagctgggctgtgtgcacgaaccccccgttcagcccgaccgctgcgccttatccggtaac
tatcgtcttgagtccaacccggtaagacacgacttatcgccactggcagcagccactggtaacaggat
tagcagagcgaggtatgtaggcggtgctacagagttcttgaagtggtggcctaactacggctacacta
gaagaacagtatttggtatctgcgctctgctgaagccagttaccttcggaaaaagagttggtagctct
tgatccggcaaacaaaccaccgctggtagcggtttttttgtttgcaagcagcagattacgcgcagaaa
aaaaggatctcaagaagatcctttgatcttttctacggggtctgacgctcagtggaacgaaaactcac
gttaagggattttggtcatgagattatcaaaaaggatcttcacctagatccttttaaattaaaaatga
agttttaaatcaatctaaagtatatatgagtaaacttggtctgacagttaccaatgcttaatcagtga
ggcacctatctcagcgatctgtctatttcgttcatccatagttgcctgactccccgtcgtgtagataa
ctacgatacgggagggcttaccatctggccccagtgctgcaatgataccgcgagacccacgctcaccg
gctccagatttatcagcaataaaccagccagccggaagggccgagcgcagaagtggtcctgcaacttt
atccgcctccatccagtctattaattgttgccgggaagctagagtaagtagttcgccagttaatagtt
tgcgcaacgttgttgccattgctacaggcatcgtggtgtcacgctcgtcgtttggtatggcttcattc
agctccggttcccaacgatcaaggcgagttacatgatcccccatgttgtgcaaaaaagcggttagctc
cttcggtcctccgatcgttgtcagaagtaagttggccgcagtgttatcactcatggttatggcagcac
tgcataattctcttactgtcatgccatccgtaagatgcttttctgtgactggtgagtactcaaccaag
tcattctgagaatagtgtatgcggcgaccgagttgctcttgcccggcgtcaatacgggataataccgc
gccacatagcagaactttaaaagtgctcatcattggaaaacgttcttcggggcgaaaactctcaagga
tcttaccgctgttgagatccagttcgatgtaacccactcgtgcacccaactgatcttcagcatctttt
actttcaccagcgtttctgggtgagcaaaaacaggaaggcaaaatgccgcaaaaaagggaataagggc
gacacggaaatgttgaatactcatactcttcctttttcaatattattgaagcatttatcagggttatt
gtctcatgagcggatacatatttgaatgtatttagaaaaataaacaaataggggttccgcgcacattt
ccccgaaaagtgccacctgacgtc
pMD141-pcDNA3.1-SARS2_mut_v4-GGSG-foldon-G-6xHis-BirA-T2A-mScarlet
SEQ ID NO: 118
gacggatcgggagatctcccgatcccctatggtgcactctcagtacaatctgctctgatgccgcatag
ttaagccagtatctgctccctgcttgtgtgttggaggtcgctgagtagtgcgcgagcaaaatttaagc
tacaacaaggcaaggcttgaccgacaattgcatgaagaatctgcttagggttaggcgttttgcgctgc
ttcgcgatgtacgggccagatatacgcgttgacattgattattgactagttattaatagtaatcaatt
acggggtcattagttcatagcccatatatggagttccgcgttacataacttacggtaaatggcccgcc
tggctgaccgcccaacgacccccgcccattgacgtcaataatgacgtatgttcccatagtaacgccaa
tagggactttccattgacgtcaatgggtggagtatttacggtaaactgcccacttggcagtacatcaa
gtgtatcatatgccaagtacgccccctattgacgtcaatgacggtaaatggcccgcctggcattatgc
ccagtacatgaccttatgggactttcctacttggcagtacatctacgtattagtcatcgctattacca
tggtgatgcggttttggcagtacatcaatgggcgtggatagcggtttgactcacggggatttccaagt
ctccaccccattgacgtcaatgggagtttgttttggcaccaaaatcaacgggactttccaaaatgtcg
taacaactccgccccattgacgcaaatgggcggtaggcgtgtacggtgggaggtctatataagcagag
ctctctggctaactagagaacccactgcttactggcttatcgaaattaatacgactcactatagggag
acccaagctggATGTTTGTGTTCCTGGTGCTGCTGCCACTGGTGTCCAGCCAGTGTGTGAACCTGACC
ACCAGGACCCAACTTCCTCCTGCCTACACCAACTCCTTCACCAGGGGAGTCTACTACCCTGACAAGGT
GTTCAGGTCCTCTGTGCTGCACAGCACCCAGGACCTGTTCCTGCCATTCTTCAGCAATGTGACCTGGT
TCCATGCCATCCATGTGTCTGGCACCAATGGCACCAAGAGGTTTGACAACCCTGTGCTGCCATTCAAT
GATGGAGTCTACTTTGCCAGCACAGAGAAGAGCAACATCATCAGGGGCTGGATTTTTGGCACCACCCT
GGACAGCAAGACCCAGTCCCTGCTGATTGTGAACAATGCCACCAATGTGGTGATTAAGGTGTGTGAGT
TCCAGTTCTGTAATGACCCATTCCTGGGAGTCTACTACCACAAGAACAACAAGTCCTGGATGGAGTCT
GAGTTCAGGGTCTACTCCTCTGCCAACAACTGTACCTTTGAATATGTGAGCCAACCATTCCTGATGGA
CTTGGAGGGCAAGCAGGGCAACTTCAAGAACCTGAGGGAGTTTGTGTTCAAGAACATTGATGGCTACT
TCAAGATTTACAGCAAACACACACCAATCAACCTGGTGAGGGACCTGCCACAGGGCTTCTCTGCCTTG
GAACCACTGGTGGACCTGCCAATTGGCATCAACATCACCAGGTTCCAGACCCTGCTGGCTCTGCACAG
GTCCTACCTGACACCTGGAGACTCCTCCTCTGGCTGGACAGCAGGAGCAGCAGCCTACTATGTGGGCT
ACCTCCAACCAAGGACCTTCCTGCTGAAATACAATGAGAATGGCACCATCACAGATGCTGAGGGCAAG
CAGGGCAACTTCAAGAACCTGAGGGAGTTTGTGTTCAAGAACATTGATGGCTACTTCAAGATTTACAG
CAAACACACACCAATCAACCTGGTGAGGGACCTGCCACAGGGCTTCTCTGCCTTGGAACCACTGGTGG
ACCTGCCAATTGGCATCAACATCACCAGGTTCCAGACCCTGCTGGCTCTGCACAGGTCCTACCTGACA
CCTGGAGACTCCTCCTCTGGCTGGACAGCAGGAGCAGCAGCCTACTATGTGGGCTACCTCCAACCAAG
GACCTTCCTGCTGAAATACAATGAGAATGGCACCATCACAGATGCTGTGGACTGTGCCCTGGACCCAC
TGTCTGAGACCAAGTGTACCCTGAAATCCTTCACAGTGGAGAAGGGCATCTACCAGACCAGCAACTTC
AGGGTCCAACCAACAGAGAGCATTGTGAGGTTTCCAAACATCACCAACCTGTGTCCATTTGGAGAGGT
GTTCAATGCCACCAGGTTTGCCTCTGTCTATGCCTGGAACAGGAAGAGGATTAGCAACTGTGTGGCTG
ACTACTCTGTGCTCTACAACTCTGCCTCCTTCAGCACCTTCAAGTGTTATGGAGTGAGCCCAACCAAA
CTGAATGACCTGTGTTTCACCAATGTCTATGCTGACTCCTTTGTGATTAGGGGAGATGAGGTGAGACA
GATTGCCCCTGGACAAACAGGCgccATTGCTGACTACAACTACAAACTGCCTGATGACTTCACAGGCT
GTGTGATTGCCTGGAACAGCgccAACCTGGACAGCAAGGTGGGAGGCAACgccAACTACCTCTACAGA
gccgctAGGAAGAGCAACCTGAAACCATTTGAGAGGGACATCAGCACAGAGATTgccgctGCTGGCAG
CACACCATGTAATGGAGTGGAGGGCgccgctTGTTACTTTCCACTCgccTCCTATGGCTTCgccCCAg
ctgccGGAGTGGGCTACCAACCATACAGGGTGGTGGTGCTGTCCTTTGAACTGCTCCATGCCCCTGCC
ACAGTGTGTGGACCAAAGAAGAGCACCAACCTGGTGAAGAACAAGTGTGTGAACTTCAACgccAATGG
ACTGACAGGCACAGGAGTGCTGACAGAGAGCAACAAGAAGTTCCTGCCATTCCAACAGTTTGGCAGGG
ACATTGCTGACACCACAGATGCTGTGAGGGACCCACAGACCTTGGAGATTCTGGACATCACACCATGT
TCCTTTGGAGGAGTGTCTGTGATTACACCTGGCACCAACACCAGCAACCAGGTGGCTGTGCTCTACCA
GGATGTGAACTGTACTGAGGTGCCTGTGGCTATCCATGCTGACCAACTTACACCAACCTGGAGGGTCT
ACAGCACAGGCAGCAATGTGTTCCAGACCAGGGCTGGCTGTCTGATTGGAGCAGAGCATGTGAACAAC
TCCTATGAGTGTGACATCCCAATTGGAGCAGGCATCTGTGCCTCCTACCAGACCCAGACCAACAGCCC
AAGGAGGGCAAGGTCTGTGGCAAGCCAGAGCATCATTGCCTACACAATGAGTCTGGGAGCAGAGAACT
CTGTGGCTTACAGCAACAACAGCATTGCCATCCCAACCAACTTCACCATCTCTGTGACCACAGAGATT
CTGCCTGTGAGTATGACCAAGACCTCTGTGGACTGTACAATGTATATCTGTGGAGACAGCACAGAGTG
TAGCAACCTGCTGCTCCAATATGGCTCCTTCTGTACCCAACTTAACAGGGCTCTGACAGGCATTGCTG
TGGAACAGGACAAGAACACCCAGGAGGTGTTTGCCCAGGTGAAGCAGATTTACAAGACACCTCCAATC
AAGGACTTTGGAGGCTTCAACTTCAGCCAGATTCTGCCTGACCCAAGCAAGCCAAGCAAGAGGTCCTT
CATTGAGGACCTGCTGTTCAACAAGGTGACCCTGGCTGATGCTGGCTTCATCAAGCAATATGGAGACT
GTCTGGGAGACATTGCTGCCAGGGACCTGATTTGTGCCCAGAAGTTCAATGGACTGACAGTGCTGCCT
CCACTGCTGACAGATGAGATGATTGCCCAATACACCTCTGCCCTGCTGGCTGGCACCATCACCTCTGG
CTGGACCTTTGGAGCAGGAGCAGCCCTCCAAATCCCATTTGCTATGCAGATGGCTTACAGGTTCAATG
GCATTGGAGTGACCCAGAATGTGCTCTATGAGAACCAGAAACTGATTGCCAACCAGTTCAACTCTGCC
ATTGGCAAGATTCAGGACTCCCTGTCCAGCACAGCCTCTGCCCTGGGCAAACTCCAAGATGTGGTGAA
CCAGAATGCCCAGGCTCTGAACACCCTGGTGAAGCAACTTTCCAGCAACTTTGGAGCCATCTCCTCTG
TGCTGAATGACATCCTGAGCAGACTGGACcctcccGAGGCTGAGGTCCAGATTGACAGACTGATTACA
GGCAGACTCCAATCCCTCCAAACCTATGTGACCCAACAACTTATCAGGGCTGCTGAGATTAGGGCATC
TGCCAACCTGGCTGCCACCAAGATGAGTGAGTGTGTGCTGGGACAAAGCAAGAGGGTGGACTTCTGTG
GCAAGGGCTACCACCTGATGAGTTTTCCACAGTCTGCCCCTCATGGAGTGGTGTTCCTGCATGTGACC
TATGTGCCTGCCCAGGAGAAGAACTTCACCACAGCCCCTGCCATCTGCCATGATGGCAAGGCTCACTT
TCCAAGGGAGGGAGTGTTTGTGAGCAATGGCACCCACTGGTTTGTGACCCAGAGGAACTTCTATGAAC
CACAGATTATCACCACAGACAACACCTTTGTGTCTGGCAACTGTGATGTGGTGATTGGCATTGTGAAC
AACACAGTCTATGACCCACTCCAACCTGAACTGGACTCCTTCAAGGAGGAACTGGACAAATACTTCAA
GAACCACACCAGCCCTGATGTGGACCTGGGAGACATCTCTGGCATCAATGCCTCTGTGGTGAACATCC
AGAAGGAGATTGACAGACTGAATGAGGTGGCTAAGAACCTGAATGAGTCCCTGATTGACCTCCAAGAA
CTGGGCAAAggcggaggcagcgggTATATTCCGGAGGCTCCAAGAGACGGTCAGGCGTACGTACGGAA
GGACGGCGAATGGGTTCTCTTGAGCACTTTCTTAggcCATCATCATCACCACCATATGAAGGACAACA
CAGTCCCGCTGAAACTTATTGCGCTTCTCGCAAATGGAGAGTTCCACTCTGGAGAGCAACTCGGCGAG
ACCCTGGGAATGAGTCGGGCGGCTATCAACAAGCATATACAGACTTTGAGGGATTGGGGTGTGGATGT
CTTCACTGTTCCGGGTAAGGGCTATTCACTGCCCGAACCGATCCAACTTCTCAATGCCAAACAGATAT
TGGGGCAACTTGATGGTGGCTCTGTCGCCGTGTTGCCGGTCATCGATAGCACGAACCAATATCTCTTG
GACCGCATCGGAGAACTCAAGAGCGGAGACGCATGTATAGCGGAATATCAGCAAGCCGGTAGAGGAAG
AAGGGGACGGAAATGGTTCTCTCCATTCGGAGCCAACCTGTATTTGAGCATGTTCTGGCGATTGGAGC
AGGGACCAGCCGCCGCCATCGGTCTCTCTCTTGTCATAGGTATTGTCATGGCGGAAGTCCTCCGCAAA
CTCGGGGCCGACAAAGTACGCGTGAAATGGCCGAACGACCTCTATCTTCAGGATAGGAAATTGGCAGG
GATATTGGTTGAACTGACGGGTAAGACGGGGGACGCCGCTCAGATTGTTATAGGTGCCGGAATAAACA
TGGCCATGCGCAGAGTTGAAGAGTCAGTGGTAAACCAGGGTTGGATCACTCTCCAAGAGGCGGGAATA
AACCTGGATCGCAACACACTGGCTGCCATGCTTATACGGGAACTCCGAGCTGCATTGGAACTGTTCGA
GCAAGAAGGGCTTGCTCCGTACTTGAGCAGGTGGGAAAAGCTGGATAATTTCATAAATCGCCCTGTTA
AACTCATCATCGGAGATAAAGAGATTTTTGGCATAAGTCGAGGAATCGATAAACAAGGTGCATTGCTG
CTTGAGCAAGACGGTATAATCAAACCTTGGATGGGTGGCGAAATCTCACTTAGAAGCGCGGAAAAATA
TGAACAAggcagtggagagggcagaggaagtctgctaacatgcggtgacgtcgaggagaatcctggcc
caatgagtaaaggagaagctgtgattaaagagttcatgcgcttcaaagttcacatggagggttctatg
aacggtcacgagttcgagatcgaaggcgaaggcgagggccgtccgtatgaaggcacccagaccgccaa
actgaaagtgactaaaggcggcccgctgcctttttcctgggacatcctgagcccgcaatttatgtacg
gttctagggcgttcaccaaacacccagcggatatcccggactattataagcagtcttttccggaaggt
ttcaagtgggaacgcgtaatgaattttgaagatggtggtgccgtgaccgtcactcaggacacctccct
ggaggatggcaccctgatctataaagttaaactgcgtggtactaattttccacctgatggcccggtga
tgcagaaaaagacgatgggttgggaggcgtctaccgaacgcttgtatccggaagatggtgtgctgaaa
ggcgacattaaaatggccctgcgcctgaaagatggcggccgctatctggctgacttcaaaaccacgta
caaagccaagaaacctgtgcagatgcctggcgcgtacaatgtggaccgcaaactggacatcacctctc
ataatgaagattatacggtggtagagcaatatgagcgctccgagggtcgtcattctaccggtggcatg
gatgaactatacaaataagtttaaacccgctgatcagcctcgactgtgccttctagttgccagccatc
tgttgtttgcccctcccccgtgccttccttgaccctggaaggtgccactcccactgtcctttcctaat
aaaatgaggaaattgcatcgcattgtctgagtaggtgtcattctattctggggggtggggtggggcag
gacagcaagggggaggattgggaagacaatagcaggcatgctggggatgcggtgggctctatggcttc
tgaggcggaaagaaccagctggggctctagggggtatccccacgcgccctgtagcggcgcattaagcg
cggcgggtgtggtggttacgcgcagcgtgaccgctacacttgccagcgccctagcgcccgctcctttc
gctttcttcccttcctttctcgccacgttcgccggctttccccgtcaagctctaaatcgggggctccc
tttagggttccgatttagtgctttacggcacctcgaccccaaaaaacttgattagggtgatggttcac
gtagtgggccatcgccctgatagacggtttttcgccctttgacgttggagtccacgttctttaatagt
ggactcttgttccaaactggaacaacactcaaccctatctcggtctattcttttgatttataagggat
tttgccgatttcggcctattggttaaaaaatgagctgatttaacaaaaatttaacgcgaattaattct
gtggaatgtgtgtcagttagggtgtggaaagtccccaggctccccagcaggcagaagtatgcaaagca
tgcatctcaattagtcagcaaccaggtgtggaaagtccccaggctccccagcaggcagaagtatgcaa
agcatgcatctcaattagtcagcaaccatagtcccgcccctaactccgcccatcccgcccctaactcc
gcccagttccgcccattctccgccccatggctgactaattttttttatttatgcagaggccgaggccg
cctctgcctctgagctattccagaagtagtgaggaggcttttttggaggcctaggcttttgcaaaaag
ctcccgggagcttgtatatccattttcggatctgatcaagagacaggatgaggatcgtttcgcatgat
tgaacaagatggattgcacgcaggttctccggccgcttgggtggagaggctattcggctatgactggg
cacaacagacaatcggctgctctgatgccgccgtgttccggctgtcagcgcaggggcgcccggttctt
tttgtcaagaccgacctgtccggtgccctgaatgaactgcaggacgaggcagcgcggctatcgtggct
ggccacgacgggcgttccttgcgcagctgtgctcgacgttgtcactgaagcgggaagggactggctgc
tattgggcgaagtgccggggcaggatctcctgtcatctcaccttgctcctgccgagaaagtatccatc
atggctgatgcaatgcggcggctgcatacgcttgatccggctacctgcccattcgaccaccaagcgaa
acatcgcatcgagcgagcacgtactcggatggaagccggtcttgtcgatcaggatgatctggacgaag
agcatcaggggctcgcgccagccgaactgttcgccaggctcaaggcgcgcatgcccgacggcgaggat
ctcgtcgtgacccatggcgatgcctgcttgccgaatatcatggtggaaaatggccgcttttctggatt
catcgactgtggccggctgggtgtggcggaccgctatcaggacatagcgttggctacccgtgatattg
ctgaagagcttggcggcgaatgggctgaccgcttcctcgtgctttacggtatcgccgctcccgattcg
cagcgcatcgccttctatcgccttcttgacgagttcttctgagcgggactctggggttcgaaatgacc
gaccaagcgacgcccaacctgccatcacgagatttcgattccaccgccgccttctatgaaaggttggg
cttcggaatcgttttccgggacgccggctggatgatcctccagcgcggggatctcatgctggagttct
tcgcccaccccaacttgtttattgcagcttataatggttacaaataaagcaatagcatcacaaatttc
acaaataaagcatttttttcactgcattctagttgtggtttgtccaaactcatcaatgtatcttatca
tgtctgtataccgtcgacctctagctagagcttggcgtaatcatggtcatagctgtttcctgtgtgaa
attgttatccgctcacaattccacacaacatacgagccggaagcataaagtgtaaagcctggggtgcc
taatgagtgagctaactcacattaattgcgttgcgctcactgcccgctttccagtcgggaaacctgtc
gtgccagctgcattaatgaatcggccaacgcgcggggagaggcggtttgcgtattgggcgctcttccg
cttcctcgctcactgactcgctgcgctcggtcgttcggctgcggcgagcggtatcagctcactcaaag
gcggtaatacggttatccacagaatcaggggataacgcaggaaagaacatgtgagcaaaaggccagca
aaaggccaggaaccgtaaaaaggccgcgttgctggcgtttttccataggctccgcccccctgacgagc
atcacaaaaatcgacgctcaagtcagaggtggcgaaacccgacaggactataaagataccaggcgttt
ccccctggaagctccctcgtgcgctctcctgttccgaccctgccgcttaccggatacctgtccgcctt
tctcccttcgggaagcgtggcgctttctcatagctcacgctgtaggtatctcagttcggtgtaggtcg
ttcgctccaagctgggctgtgtgcacgaaccccccgttcagcccgaccgctgcgccttatccggtaac
tatcgtcttgagtccaacccggtaagacacgacttatcgccactggcagcagccactggtaacaggat
tagcagagcgaggtatgtaggcggtgctacagagttcttgaagtggtggcctaactacggctacacta
gaagaacagtatttggtatctgcgctctgctgaagccagttaccttcggaaaaagagttggtagctct
tgatccggcaaacaaaccaccgctggtagcggtttttttgtttgcaagcagcagattacgcgcagaaa
aaaaggatctcaagaagatcctttgatcttttctacggggtctgacgctcagtggaacgaaaactcac
gttaagggattttggtcatgagattatcaaaaaggatcttcacctagatccttttaaattaaaaatga
agttttaaatcaatctaaagtatatatgagtaaacttggtctgacagttaccaatgcttaatcagtga
ggcacctatctcagcgatctgtctatttcgttcatccatagttgcctgactccccgtcgtgtagataa
ctacgatacgggagggcttaccatctggccccagtgctgcaatgataccgcgagacccacgctcaccg
gctccagatttatcagcaataaaccagccagccggaagggccgagcgcagaagtggtcctgcaacttt
atccgcctccatccagtctattaattgttgccgggaagctagagtaagtagttcgccagttaatagtt
tgcgcaacgttgttgccattgctacaggcatcgtggtgtcacgctcgtcgtttggtatggcttcattc
agctccggttcccaacgatcaaggcgagttacatgatcccccatgttgtgcaaaaaagcggttagctc
cttcggtcctccgatcgttgtcagaagtaagttggccgcagtgttatcactcatggttatggcagcac
tgcataattctcttactgtcatgccatccgtaagatgcttttctgtgactggtgagtactcaaccaag
tcattctgagaatagtgtatgcggcgaccgagttgctcttgcccggcgtcaatacgggataataccgc
gccacatagcagaactttaaaagtgctcatcattggaaaacgttcttcggggcgaaaactctcaagga
tcttaccgctgttgagatccagttcgatgtaacccactcgtgcacccaactgatcttcagcatctttt
actttcaccagcgtttctgggtgagcaaaaacaggaaggcaaaatgccgcaaaaaagggaataagggc
gacacggaaatgttgaatactcatactcttcctttttcaatattattgaagcatttatcagggttatt
gtctcatgagcggatacatatttgaatgtatttagaaaaataaacaaataggggttccgcgcacattt
ccccgaaaagtgccacctgacgtc
pMD142-MSCV-hACE2-T2A-BFP-3xFLAG
SEQ ID NO: 119
gaccccacctgtaggtttggcaagctagcttaagtaacgccattttgcaaggcatggaaaatacataa
ctgagaatagagaagttcagatcaaggttaggaacagagagacagcagaatatgggccaaacaggata
tctgtggtaagcagttcctgccccggctcagggccaagaacagatggtccccagatgcggtcccgccc
tcagcagtttctagagaaccatcagatgtttccagggtgccccaaggacctgaaatgaccctgtgcct
tatttgaactaaccaatcagttcgcttctcgcttctgttcgcgcgcttctgctccccgagctcaataa
aagagcccacaacccctcactcggcgcgccagtcctccgatagactgcgtcgcccgggtacccgtatt
cccaataaagcctcttgctgtttgcatccgaatcgtggactcgctgatccttgggagggtctcctcag
attgattgactgcccacctcgggggtctttcatttggaggttccaccgagatttggagacccctgccc
agggaccaccgacccccccgccgggaggtaagctggccagcggtcgtttcgtgtctgtctctgtcttt
gtgcgtgtttgtgccggcatctaatgtttgcgcctgcgtctgtactagttagctaactagctctgtat
ctggcggacccgtggtggaactgacgagttctgaacacccggccgcaaccctgggagacgtcccaggg
actttgggggccgtttttgtggcccgacctgaggaagggagtcgatgtggaatccgaccccgtcagga
tatgtggttctggtaggagacgagaacctaaaacagttcccgcctccgtctgaatttttgctttcggt
ttggaaccgaagccgcgcgtcttgtctgctgcagcgctgcagcatcgttctgtgttgtctctgtctga
ctgtgtttctgtatttgtctgaaaattagggccagactgttaccactcccttaagtttgaccttaggt
cactggaaagatgtcgagcggatcgctcacaaccagtcggtagatgtcaagaagagacgttgggttac
cttctgctctgcagaatggccaacctttaacgtcggatggccgcgagacggcacctttaaccgagacc
tcatcacccaggttaagatcaaggtcttttcacctggcccgcatggacacccagaccaggtcccctac
atcgtgacctgggaagccttggcttttgacccccctccctgggtcaagccctttgtacaccctaagcc
tccgcctcctcttcctccatccgccccgtctctcccccttgaacctcctcgttcgaccccgcctcgat
cctccctttatccagccctcactccttctctaggcgccggaattagatctctcgagatcgatgccttg
agacaaatggcagtattcatccacaattttaaaagaaaaggggggattggggggtacagtgcagggga
aagaatagtagacataatagcaacagacatacaaactaaagaattacaaaaacaaattacaaaaattc
aaaattttcgggtttattacagggacagcagagatccactttggcgccggctcgagggggcccgggga
attcgctagctaggtcttgaaaggagtgggaattggctccggtgcccgtcagtgggcagagcgcacat
cgcccacagtccccgagaagttggggggaggggtcggcaattgatccggtgcctagagaaggtggcgc
ggggtaaactgggaaagtgatgtcgtgtactggctccgcctttttcccgagggtgggggagaaccgta
tataagtgcagtagtcgccgtgaacgttctttttcgcaacgggtttgccgccagaacacaggaccggt
tctagacgtacggccaccATGCCACTGCTGCTCTTGCTGCCTCTGCTTTGGGCTGGAGCTCTGGCTGA
TTACAAGGATGACGACGATAAGGGTGGAGGCGGTAGCCAGTCCACCATTGAGGAACAGGCCAAGACAT
TTTTGGACAAGTTTAACCACGAAGCCGAAGACCTGTTCTATCAAAGTTCACTTGCTTCTTGGAATTAT
AACACCAATATTACTGAAGAGAATGTCCAAAACATGAATAATGCTGGGGACAAATGGTCTGCCTTTTT
AAAGGAACAGTCCACACTTGCCCAAATGTATCCACTACAAGAAATTCAGAATCTCACAGTCAAGCTTC
AGCTGCAGGCTCTTCAGCAAAATGGGTCTTCAGTGCTCTCAGAAGACAAGAGCAAACGGTTGAACACA
ATTCTAAATACAATGAGCACCATCTACAGTACTGGAAAAGTTTGTAACCCAGATAATCCACAAGAATG
CTTATTACTTGAACCAGGTTTGAATGAAATAATGGCAAACAGTTTAGACTACAATGAGAGGCTCTGGG
CTTGGGAAAGCTGGAGATCTGAGGTCGGCAAGCAGCTGAGGCCATTATATGAAGAGTATGTGGTCTTG
AAAAATGAGATGGCAAGAGCAAATCATTATGAGGACTATGGGGATTATTGGAGAGGAGACTATGAAGT
AAATGGGGTAGATGGCTATGACTACAGCCGCGGCCAGTTGATTGAAGATGTGGAACATACCTTTGAAG
AGATTAAACCATTATATGAACATCTTCATGCCTATGTGAGGGCAAAGTTGATGAATGCCTATCCTTCC
TATATCAGTCCAATTGGATGCCTCCCTGCTCATTTGCTTGGTGATATGTGGGGTAGATTTTGGACAAA
TCTGTACTCTTTGACAGTTCCCTTTGGACAGAAACCAAACATAGATGTTACTGATGCAATGGTGGACC
AGGCCTGGGATGCACAGAGAATATTCAAGGAGGCCGAGAAGTTCTTTGTATCTGTTGGTCTTCCTAAT
ATGACTCAAGGATTCTGGGAAAATTCCATGCTAACGGACCCAGGAAATGTTCAGAAAGCAGTCTGCCA
TCCCACAGCTTGGGACCTGGGGAAGGGCGACTTCAGGATCCTTATGTGCACAAAGGTGACAATGGACG
ACTTCCTGACAGCTCATCATGAGATGGGGCATATCCAGTATGATATGGCATATGCTGCACAACCTTTT
CTGCTAAGAAATGGAGCTAATGAAGGATTCCATGAAGCTGTTGGGGAAATCATGTCACTTTCTGCAGC
CACACCTAAGCATTTAAAATCCATTGGTCTTCTGTCACCCGATTTTCAAGAAGACAATGAAACAGAAA
TAAACTTCCTGCTCAAACAAGCACTCACGATTGTTGGGACTCTGCCATTTACTTACATGTTAGAGAAG
TGGAGGTGGATGGTCTTTAAAGGGGAAATTCCCAAAGACCAGTGGATGAAAAAGTGGTGGGAGATGAA
GCGAGAGATAGTTGGGGTGGTGGAACCTGTGCCCCATGATGAAACATACTGTGACCCCGCATCTCTGT
TCCATGTTTCTAATGATTACTCATTCATTCGATATTACACAAGGACCCTTTACCAATTCCAGTTTCAA
GAAGCACTTTGTCAAGCAGCTAAACATGAAGGCCCTCTGCACAAATGTGACATCTCAAACTCTACAGA
AGCTGGACAGAAACTGTTCAATATGCTGAGGCTTGGAAAATCAGAACCCTGGACCCTAGCATTGGAAA
ATGTTGTAGGAGCAAAGAACATGAATGTAAGGCCACTGCTCAACTACTTTGAGCCCTTATTTACCTGG
CTGAAAGACCAGAACAAGAATTCTTTTGTGGGATGGAGTACCGACTGGAGTCCATATGCAGACCAAAG
CATCAAAGTGAGGATAAGCCTAAAATCAGCTCTTGGAGATAAAGCATATGAATGGAACGACAATGAAA
TGTACCTGTTCCGATCATCTGTTGCATATGCTATGAGGCAGTACTTTTTAAAAGTAAAAAATCAGATG
ATTCTTTTTGGGGAGGAGGATGTGCGAGTGGCTAATTTGAAACCAAGAATCTCCTTTAATTTCTTTGT
CACTGCACCTAAAAATGTGTCTGATATCATTCCTAGAACTGAAGTTGAAAAGGCCATCAGGATGTCCC
GGAGCCGTATCAATGATGCTTTCCGTCTGAATGACAACAGCCTAGAGTTTCTGGGGATACAGCCAACA
CTTGGACCTCCTAACCAGCCCCCTGTTTCCATATGGCTGATTGTTTTTGGAGTTGTGATGGGAGTGAT
AGTGGTTGGCATTGTCATCCTGATCTTCACTGGGATCAGAGATCGGAAGAAGAAAAATAAAGCAAGAA
GTGGAGAAAATCCTTATGCCTCCATCGATATTAGCAAAGGAGAAAATAATCCAGGATTCCAAAACACT
GATGATGTTCAGACCTCCTTTttcgaaggatccggcgcaacaaacttctctctgctgaaacaagccgg
agatgtcgaagagaatcctggaccgatggtgtctaagggcgaagagctgattaaggagaacatgcaca
tgaagctgtacatggagggcaccgtggacaaccatcacttcaagtgcacatccgagggcgaaggcaag
ccctacgagggcacccagaccatgagaatcaaggtggtcgagggcggccctctccccttcgccttcga
catcctggctactagcttcctctacggcagcaagaccttcatcaaccacacccagggcatccccgact
tcttcaagcagtccttccctgagggcttcacatgggagagagtcaccacatacgaagatgggggcgtg
ctgaccgctacccaggacaccagcctccaggacggctgcctcatctacaacgtcaagatcagaggggt
gaacttcacatccaacggccctgtgatgcagaagaaaacactcggctgggaggccttcaccgagacgc
tgtaccccgctgacggcggcctggaaggcagaaacgacatggccctgaagctcgtgggcgggagccat
ctgatcgcaaacgccaagaccacatatagatccaagaaacccgctaagaacctcaagatgcctggcgt
ctactatgtggactacagactggaaagaatcaaggaggccaacaacgagacctacgtcgagcagcacg
aggtggcagtggccagatactgcgacctccctagcaaactggggcacaagcttaatacgcgtgactac
aaagaccatgacggtgattataaagatcatgacatcgattacaaggatgacgatgacaagtaaggatc
caataaaagatctttattttcattagatctgtgtgttggttttttgtgtcgataaaataaaagatttt
atttagtctccagaaaaaggggggaatgaaagaccccacctgtaggtttggcaagctagcttaagtaa
cgccattttgcaaggcatggaaaatacataactgagaatagagaagttcagatcaaggttaggaacag
agagacagcagaatatgggccaaacaggatatctgtggtaagcagttcctgccccggctcagggccaa
gaacagatggtccccagatgcggtcccgccctcagcagtttctagagaaccatcagatgtttccaggg
tgccccaaggacctgaaatgaccctgtgccttatttgaactaaccaatcagttcgcttctcgcttctg
ttcgcgcgcttctgctccccgagctcaataaaagagcccacaacccctcactcggcgcgccagtcctc
cgatagactgcgtcgcccgggtacccgtgtatccaataaaccctcttgcagttgcatccgacttgtgg
tctcgctgttccttgggagggtctcctctgagtgattgactacccgtcagcgggggtctttcatgggt
aacagtttcttgaagttggagaacaacattctgagggtaggagtcgaatattaagtaatcctgactca
attagccactgttttgaatccacatactccaatactcctgaaatagttcattatggacagcgcagaaa
gagctggggagaattgtgaaattgttatccgctcacaattccacacaacatacgagccggaagcataa
agtgtaaagcctggggtgcctaatgagtgagctaactcacattaattgcgttgcgctcactgcccgct
ttccagtcgggaaacctgtcgtgccagctgcattaatgaatcggccaacgcgcggggagaggcggttt
gcgtattgggcgctcttccgcttcctcgctcactgactcgctgcgctcggtcgttcggctgcggcgag
cggtatcagctcactcaaaggcggtaatacggttatccacagaatcaggggataacgcaggaaagaac
atgtgagcaaaaggccagcaaaaggccaggaaccgtaaaaaggccgcgttgctggcgtttttccatag
gctccgcccccctgacgagcatcacaaaaatcgacgctcaagtcagaggtggcgaaacccgacaggac
tataaagataccaggcgtttccccctggaagctccctcgtgcgctctcctgttccgaccctgccgctt
accggatacctgtccgcctttctcccttcgggaagcgtggcgctttctcatagctcacgctgtaggta
tctcagttcggtgtaggtcgttcgctccaagctgggctgtgtgcacgaaccccccgttcagcccgacc
gctgcgccttatccggtaactatcgtcttgagtccaacccggtaagacacgacttatcgccactggca
gcagccactggtaacaggattagcagagcgaggtatgtaggcggtgctacagagttcttgaagtggtg
gcctaactacggctacactagaagaacagtatttggtatctgcgctctgctgaagccagttaccttcg
gaaaaagagttggtagctcttgatccggcaaacaaaccaccgctggtagcggtggtttttttgtttgc
aagcagcagattacgcgcagaaaaaaaggatctcaagaagatcctttgatcttttctacggggtctga
cgctcagtggaacgaaaactcacgttaagggattttggtcatgagattatcaaaaaggatcttcacct
agatccttttaaattaaaaatgaagttttaaatcaatctaaagtatatatgagtaaacttggtctgac
agttaccaatgcttaatcagtgaggcacctatctcagcgatctgtctatttcgttcatccatagttgc
ctgactccccgtcgtgtagataactacgatacgggagggcttaccatctggccccagtgctgcaatga
taccgcgagacccacgctcaccggctccagatttatcagcaataaaccagccagccggaagggccgag
cgcagaagtggtcctgcaactttatccgcctccatccagtctattaattgttgccgggaagctagagt
aagtagttcgccagttaatagtttgcgcaacgttgttgccattgctacaggcatcgtggtgtcacgct
cgtcgtttggtatggcttcattcagctccggttcccaacgatcaaggcgagttacatgatcccccatg
ttgtgcaaaaaagcggttagctccttcggtcctccgatcgttgtcagaagtaagttggccgcagtgtt
atcactcatggttatggcagcactgcataattctcttactgtcatgccatccgtaagatgcttttctg
tgactggtgagtactcaaccaagtcattctgagaatagtgtatgcggcgaccgagttgctcttgcccg
gcgtcaatacgggataataccgcgccacatagcagaactttaaaagtgctcatcattggaaaacgttc
ttcggggcgaaaactctcaaggatcttaccgctgttgagatccagttcgatgtaacccactcgtgcac
ccaactgatcttcagcatcttttactttcaccagcgtttctgggtgagcaaaaacaggaaggcaaaat
gccgcaaaaaagggaataagggcgacacggaaatgttgaatactcatactcttcctttttcaatatta
ttgaagcatttatcagggttattgtctcatgagcggatacatatttgaatgtatttagaaaaataaac
aaataggggttccgcgcacatttccccgaaaagtgccacctgacgtctaagaaaccattattatcatg
acattaacctataaaaataggcgtatcacgaggccctttcgtctcgcgcgtttcggtgatgacggtga
aaacctctgacacatgcagctcccggagacggtcacagcttgtctgtaagcggatgccgggagcagac
aagcccgtcagggcgcgtcagcgggtgttggcgggtgtcggggctggcttaactatgcggcatcagag
cagattgtactgagagtgcaccatatgcggtgtgaaataccgcacagatgcgtaaggagaaaataccg
catcaggcgccattcgccattcaggctgcgcaactgttgggaagggcgatcggtgcgggcctcttcgc
tattacgccagctggcgaaagggggatgtgctgcaaggcgattaagttgggtaacgccagggttttcc
cagtcacgacgttgtaaaacgacggcgcaaggaatggtgcatgcaaggagatggcgcccaacagtccc
ccggccacggggcctgccaccatacccacgccgaaacaagcgctcatgagcccgaagtggcgagcccg
atcttccccatcggtgatgtcggcgatataggcgccagcaaccgcacctgtggcgccggtgatgccgg
ccacgatgcgtccggcgtagaggcgattagtccaatttgttaaagacaggatatcagtggtccaggct
ctagttttgactcaacaatatcaccagctgaagcctatagagtacgagccatagataaaataaaagat
tttatttagtctccagaaaaaggggggaatgaaa
pMD143-pAAV-EF1a-N protein-2A-Scarlet-FLAG-BGH
SEQ ID NO: 120
cctgcaggcagctgcgcgctcgctcgctcactgaggccgcccgggcaaagcccgggcgtcgggcgacc
tttggtcgcccggcctcagtgagcgagcgagcgcgcagagagggagtggccaactccatcactagggg
ttcctgcggccgcaGCTCCGGTGCCCGTCAGTGGGCAGAGCGCACATCGCCCACAGTCCCCGAGAAGT
TGGGGGGAGGGGTCGGCAATTGAACCGGTGCCTAGAGAAGGTGGCGCGGGGTAAACTGGGAAAGTGAT
GTCGTGTACTGGCTCCGCCTTTTTCCCGAGGGTGGGGGAGAACCGTATATAAGTGCAGTAGTCGCCGT
GAACGTTCTTTTTCGCAACGGGTTTGCCGCCAGAACACAGGTAAGTGCCGTGTGTGGTTCCCGCGGGC
CTGGCCTCTTTACGGGTTATGGCCCTTGCGTGCCTTGAATTACTTCCACGCCCCTGGCTGCAGTACGT
GATTCTTGATCCCGAGCTTCGGGTTGGAAGTGGGTGGGAGAGTTCGAGGCCTTGCGCTTAAGGAGCCC
CTTCGCCTCGTGCTTGAGTTGAGGCCTGGCTTGGGCGCTGGGGCCGCCGCGTGCGAATCTGGTGGCAC
CTTCGCGCCTGTCTCGCTGCTTTCGATAAGTCTCTAGCCATTTAAAATTTTTGATGACCTGCTGCGAC
GCTTTTTTTCTGGCAAGATAGTCTTGTAAATGCGGGCCAAGATCTGCACACTGGTATTTCGGTTTTTG
GGGCCGCGGGCGGCGACGGGGCCCGTGCGTCCCAGCGCACATGTTCGGCGAGGCGGGGCCTGCGAGCG
CGGCCACCGAGAATCGGACGGGGGTAGTCTCAAGCTGGCCGGCCTGCTCTGGTGCCTGGCCTCGCGCC
GCCGTGTATCGCCCCGCCCTGGGCGGCAAGGCTGGCCCGGTCGGCACCAGTTGCGTGAGCGGAAAGAT
GGCCGCTTCCCGGCCCTGCTGCAGGGAGCTCAAAATGGAGGACGCGGCGCTCGGGAGAGCGGGCGGGT
GAGTCACCCACACAAAGGAAAAGGGCCTTTCCGTCCTCAGCCGTCGCTTCATGTGACTCCACGGAGTA
CCGGGCGCCGTCCAGGCACCTCGATTAGTTCTCGAGCTTTTGGAGTACGTCGTCTTTAGGTTGGGGGG
AGGGGTTTTATGCGATGGAGTTTCCCCACACTGAGTGGGTGGAGACTGAAGTTAGGCCAGCTTGGCAC
TTGATGTAATTCTCCTTGGAATTTGCCCTTTTTGAGTTTGGATCTTGGTTCATTCTCAAGCCTCAGAC
AGTGGTTCAAAGTTTTTTTCTTCCATTTCAGGTGTCGTGAGGATCTATTTCCGGTGAATTCGCCGCCA
CCATGAGCGATAACGGCCCCCAAAACCAACGGAATGCCCCTCGGATAACATTTGGTGGCCCTAGTGAT
TCCACAGGAAGTAACCAAAACGGAGAACGGTCTGGAGCCCGCTCTAAACAGAGGCGACCACAGGGATT
GCCAAATAACACAGCGTCATGGTTTACAGCCCTGACTCAACATGGAAAGGAGGACTTGAAATTTCCAC
GAGGACAGGGTGTACCAATAAACACAAACAGTAGTCCAGACGATCAGATCGGGTATTATAGAAGGGCC
ACGAGACGCATCAGAGGTGGAGATGGTAAAATGAAAGATCTCAGTCCGCGCTGGTACTTTTATTATTT
GGGAACCGGCCCAGAAGCGGGCCTCCCATATGGAGCCAATAAGGACGGGATTATATGGGTGGCTACAG
AGGGAGCCCTCAATACGCCAAAAGATCACATCGGAACAAGAAACCCTGCTAATAATGCCGCAATCGTG
CTGCAGTTGCCTCAGGGGACGACGCTTCCTAAAGGCTTTTACGCAGAAGGATCACGCGGCGGCAGCCA
AGCATCCTCAAGGTCTAGTTCCAGAAGTCGAAACAGCTCCAGAAACTCCACACCAGGGTCCAGTAGGG
GCACAAGTCCGGCGCGGATGGCGGGCAACGGCGGAGACGCCGCACTCGCTCTGTTGCTCCTGGACCGG
CTCAACCAACTTGAATCCAAGATGAGTGGTAAGGGACAGCAGCAACAAGGTCAAACCGTAACCAAGAA
AAGCGCTGCAGAAGCTTCTAAAAAACCTCGACAAAAACGGACCGCTACGAAGGCATATAACGTTACTC
AAGCCTTCGGGAGACGAGGGCCGGAGCAAACCCAGGGGAATTTTGGAGATCAGGAGCTCATCCGGCAA
GGGACAGATTACAAACATTGGCCTCAAATCGCTCAGTTCGCGCCGTCCGCAAGTGCTTTTTTCGGAAT
GTCTCGCATCGGTATGGAAGTAACACCATCCGGGACATGGCTCACTTATACCGGCGCCATCAAGCTGG
ACGACAAAGATCCAAATTTCAAAGATCAGGTGATTCTCTTGAATAAACACATAGACGCGTATAAGACG
TTTCCACCCACCGAACCCAAGAAGGACAAAAAGAAAAAAGCTGATGAAACACAAGCGTTGCCCCAGCG
ACAGAAGAAGCAGCAAACCGTCACCCTCCTGCCAGCCGCTGATCTCGATGACTTCTCCAAGCAACTTC
AACAGTCCATGAGCTCAGCCGATTCTACTCAGGCGggcagtggagagggcagaggaagtctgctaaca
tgcggtgacgtcgaggagaatcctggcccaatgagtaaaggagaagctgtgattaaagagttcatgcg
cttcaaagttcacatggagggttctatgaacggtcacgagttcgagatcgaaggcgaaggcgagggcc
gtccgtatgaaggcacccagaccgccaaactgaaagtgactaaaggcggcccgctgcctttttcctgg
gacatcctgagcccgcaatttatgtacggttctagggcgttcaccaaacacccagcggatatcccgga
ctattataagcagtcttttccggaaggtttcaagtgggaacgcgtaatgaattttgaagatggtggtg
ccgtgaccgtcactcaggacacctccctggaggatggcaccctgatctataaagttaaactgcgtggt
actaattttccacctgatggcccggtgatgcagaaaaagacgatgggttgggaggcgtctaccgaacg
cttgtatccggaagatggtgtgctgaaaggcgacattaaaatggccctgcgcctgaaagatggcggcc
gctatctggctgacttcaaaaccacgtacaaagccaagaaacctgtgcagatgcctggcgcgtacaat
gtggaccgcaaactggacatcacctctcataatgaagattatacggtggtagagcaatatgagcgctc
cgagggtcgtcattctaccggtggcatggatgaactatacaaataagtttaaacccgctgatcagcct
cgactgtgccttctagttgccagccatctgttgtttgcccctcccccgtgccttccttgaccctggaa
ggtgccactcccactgtcctttcctaataaaatgaggaaattgcatcgcattgtctgagtaggtgtca
ttctattctggggggtggggtggggcaggacagcaagggggaggattgggaagacaatagcaggcatg
ctggggatgcggtgggctctatgggaccgagcggccgcaggaacccctagtgatggagttggccactc
cctctctgcgcgctcgctcgctcactgaggccgggcgaccaaaggtcgcccgacgcccgggctttgcc
cgggcggcctcagtgagcgagcgagcgcgcagctgcctgcaggggcgcctgatgcggtattttctcct
tacgcatctgtgcggtatttcacaccgcatacgtcaaagcaaccatagtacgcgccctgtagcggcgc
attaagcgcggcgggtgtggtggttacgcgcagcgtgaccgctacacttgccagcgccctagcgcccg
ctcctttcgctttcttcccttcctttctcgccacgttcgccggctttccccgtcaagctctaaatcgg
gggctccctttagggttccgatttagtgctttacggcacctcgaccccaaaaaacttgatttgggtga
tggttcacgtagtgggccatcgccctgatagacggtttttcgccctttgacgttggagtccacgttct
ttaatagtggactcttgttccaaactggaacaacactcaaccctatctcgggctattcttttgattta
taagggattttgccgatttcggcctattggttaaaaaatgagctgatttaacaaaaatttaacgcgaa
ttttaacaaaatattaacgtttacaattttatggtgcactctcagtacaatctgctctgatgccgcat
agttaagccagccccgacacccgccaacacccgctgacgcgccctgacgggcttgtctgctcccggca
tccgcttacagacaagctgtgaccgtctccgggagctgcatgtgtcagaggttttcaccgtcatcacc
gaaacgcgcgagacgaaagggcctcgtgatacgcctatttttataggttaatgtcatgataataatgg
tttcttagacgtcaggtggcacttttcggggaaatgtgcgcggaacccctatttgtttatttttctaa
atacattcaaatatgtatccgctcatgagacaataaccctgataaatgcttcaataatattgaaaaag
gaagagtatgagtattcaacatttccgtgtcgcccttattcccttttttgcggcattttgccttcctg
tttttgctcacccagaaacgctggtgaaagtaaaagatgctgaagatcagttgggtgcacgagtgggt
tacatcgaactggatctcaacagcggtaagatccttgagagttttcgccccgaagaacgttttccaat
gatgagcacttttaaagttctgctatgtggcgcggtattatcccgtattgacgccgggcaagagcaac
tcggtcgccgcatacactattctcagaatgacttggttgagtactcaccagtcacagaaaagcatctt
acggatggcatgacagtaagagaattatgcagtgctgccataaccatgagtgataacactgcggccaa
cttacttctgacaacgatcggaggaccgaaggagctaaccgcttttttgcacaacatgggggatcatg
taactcgccttgatcgttgggaaccggagctgaatgaagccataccaaacgacgagcgtgacaccacg
atgcctgtagcaatggcaacaacgttgcgcaaactattaactggcgaactacttactctagcttcccg
gcaacaattaatagactggatggaggcggataaagttgcaggaccacttctgcgctcggcccttccgg
ctggctggtttattgctgataaatctggagccggtgagcgtgggtctcgcggtatcattgcagcactg
gggccagatggtaagccctcccgtatcgtagttatctacacgacggggagtcaggcaactatggatga
acgaaatagacagatcgctgagataggtgcctcactgattaagcattggtaactgtcagaccaagttt
actcatatatactttagattgatttaaaacttcatttttaatttaaaaggatctaggtgaagatcctt
tttgataatctcatgaccaaaatcccttaacgtgagttttcgttccactgagcgtcagaccccgtaga
aaagatcaaaggatcttcttgagatcctttttttctgcgcgtaatctgctgcttgcaaacaaaaaaac
caccgctaccagcggtggtttgtttgccggatcaagagctaccaactctttttccgaaggtaactggc
ttcagcagagcgcagataccaaatactgtccttctagtgtagccgtagttaggccaccacttcaagaa
ctctgtagcaccgcctacatacctcgctctgctaatcctgttaccagtggctgctgccagtggcgata
agtcgtgtcttaccgggttggactcaagacgatagttaccggataaggcgcagcggtcgggctgaacg
gggggttcgtgcacacagcccagcttggagcgaacgacctacaccgaactgagatacctacagcgtga
gctatgagaaagcgccacgcttcccgaagggagaaaggcggacaggtatccggtaagcggcagggtcg
gaacaggagagcgcacgagggagcttccagggggaaacgcctggtatctttatagtcctgtcgggttt
cgccacctctgacttgagcgtcgatttttgtgatgctcgtcaggggggcggagcctatggaaaaacgc
cagcaacgcggcctttttacggttcctggccttttgctggccttttgctcacatgt
pMD144_pAAV-EFS-S protein-TA-M protein
SEQ ID NO: 121
cctgcaggcagctgcgcgctcgctcgctcactgaggccgcccgggcaaagcccgggcgtcgggcgacc
tttggtcgcccggcctcagtgagcgagcgagcgcgcagagagggagtggccaactccatcactagggg
ttcctgcggccgcataggtcttgaaaggagtgggaattggctccggtgcccgtcagtgggcagagcgc
acatcgcccacagtccccgagaagttggggggaggggtcggcaattgatccggtgcctagagaaggtg
gcgcggggtaaactgggaaagtgatgtcgtgtactggctccgcctttttcccgagggtgggggagaac
cgtatataagtgcagtagtcgccgtgaacgttctttttcgcaacgggtttgccgccagaacacaggac
cggttctagacgtacggccaccATGTTTGTGTTCCTGGTGCTGCTGCCACTGGTGTCCAGCCAGTGTG
TGAACCTGACCACCAGGACCCAACTTCCTCCTGCCTACACCAACTCCTTCACCAGGGGAGTCTACTAC
CCTGACAAGGTGTTCAGGTCCTCTGTGCTGCACAGCACCCAGGACCTGTTCCTGCCATTCTTCAGCAA
TGTGACCTGGTTCCATGCCATCCATGTGTCTGGCACCAATGGCACCAAGAGGTTTGACAACCCTGTGC
TGCCATTCAATGATGGAGTCTACTTTGCCAGCACAGAGAAGAGCAACATCATCAGGGGCTGGATTTTT
GGCACCACCCTGGACAGCAAGACCCAGTCCCTGCTGATTGTGAACAATGCCACCAATGTGGTGATTAA
GGTGTGTGAGTTCCAGTTCTGTAATGACCCATTCCTGGGAGTCTACTACCACAAGAACAACAAGTCCT
GGATGGAGTCTGAGTTCAGGGTCTACTCCTCTGCCAACAACTGTACCTTTGAATATGTGAGCCAACCA
TTCCTGATGGACTTGGAGGGCAAGCAGGGCAACTTCAAGAACCTGAGGGAGTTTGTGTTCAAGAACAT
TGATGGCTACTTCAAGATTTACAGCAAACACACACCAATCAACCTGGTGAGGGACCTGCCACAGGGCT
TCTCTGCCTTGGAACCACTGGTGGACCTGCCAATTGGCATCAACATCACCAGGTTCCAGACCCTGCTG
GCTCTGCACAGGTCCTACCTGACACCTGGAGACTCCTCCTCTGGCTGGACAGCAGGAGCAGCAGCCTA
CTATGTGGGCTACCTCCAACCAAGGACCTTCCTGCTGAAATACAATGAGAATGGCACCATCACAGATG
CTGTGGACTGTGCCCTGGACCCACTGTCTGAGACCAAGTGTACCCTGAAATCCTTCACAGTGGAGAAG
GGCATCTACCAGACCAGCAACTTCAGGGTCCAACCAACAGAGAGCATTGTGAGGTTTCCAAACATCAC
CAACCTGTGTCCATTTGGAGAGGTGTTCAATGCCACCAGGTTTGCCTCTGTCTATGCCTGGAACAGGA
AGAGGATTAGCAACTGTGTGGCTGACTACTCTGTGCTCTACAACTCTGCCTCCTTCAGCACCTTCAAG
TGTTATGGAGTGAGCCCAACCAAACTGAATGACCTGTGTTTCACCAATGTCTATGCTGACTCCTTTGT
GATTAGGGGAGATGAGGTGAGACAGATTGCCCCTGGACAAACAGGCAAGATTGCTGACTACAACTACA
AACTGCCTGATGACTTCACAGGCTGTGTGATTGCCTGGAACAGCAACAACCTGGACAGCAAGGTGGGA
GGCAACTACAACTACCTCTACAGACTGTTCAGGAAGAGCAACCTGAAACCATTTGAGAGGGACATCAG
CACAGAGATTTACCAGGCTGGCAGCACACCATGTAATGGAGTGGAGGGCTTCAACTGTTACTTTCCAC
TCCAATCCTATGGCTTCCAACCAACCAATGGAGTGGGCTACCAACCATACAGGGTGGTGGTGCTGTCC
TTTGAACTGCTCCATGCCCCTGCCACAGTGTGTGGACCAAAGAAGAGCACCAACCTGGTGAAGAACAA
GTGTGTGAACTTCAACTTCAATGGACTGACAGGCACAGGAGTGCTGACAGAGAGCAACAAGAAGTTCC
TGCCATTCCAACAGTTTGGCAGGGACATTGCTGACACCACAGATGCTGTGAGGGACCCACAGACCTTG
GAGATTCTGGACATCACACCATGTTCCTTTGGAGGAGTGTCTGTGATTACACCTGGCACCAACACCAG
CAACCAGGTGGCTGTGCTCTACCAGGATGTGAACTGTACTGAGGTGCCTGTGGCTATCCATGCTGACC
AACTTACACCAACCTGGAGGGTCTACAGCACAGGCAGCAATGTGTTCCAGACCAGGGCTGGCTGTCTG
ATTGGAGCAGAGCATGTGAACAACTCCTATGAGTGTGACATCCCAATTGGAGCAGGCATCTGTGCCTC
CTACCAGACCCAGACCAACAGCCCAAGGAGGGCAAGGTCTGTGGCAAGCCAGAGCATCATTGCCTACA
CAATGAGTCTGGGAGCAGAGAACTCTGTGGCTTACAGCAACAACAGCATTGCCATCCCAACCAACTTC
ACCATCTCTGTGACCACAGAGATTCTGCCTGTGAGTATGACCAAGACCTCTGTGGACTGTACAATGTA
TATCTGTGGAGACAGCACAGAGTGTAGCAACCTGCTGCTCCAATATGGCTCCTTCTGTACCCAACTTA
ACAGGGCTCTGACAGGCATTGCTGTGGAACAGGACAAGAACACCCAGGAGGTGTTTGCCCAGGTGAAG
CAGATTTACAAGACACCTCCAATCAAGGACTTTGGAGGCTTCAACTTCAGCCAGATTCTGCCTGACCC
AAGCAAGCCAAGCAAGAGGTCCTTCATTGAGGACCTGCTGTTCAACAAGGTGACCCTGGCTGATGCTG
GCTTCATCAAGCAATATGGAGACTGTCTGGGAGACATTGCTGCCAGGGACCTGATTTGTGCCCAGAAG
TTCAATGGACTGACAGTGCTGCCTCCACTGCTGACAGATGAGATGATTGCCCAATACACCTCTGCCCT
GCTGGCTGGCACCATCACCTCTGGCTGGACCTTTGGAGCAGGAGCAGCCCTCCAAATCCCATTTGCTA
TGCAGATGGCTTACAGGTTCAATGGCATTGGAGTGACCCAGAATGTGCTCTATGAGAACCAGAAACTG
ATTGCCAACCAGTTCAACTCTGCCATTGGCAAGATTCAGGACTCCCTGTCCAGCACAGCCTCTGCCCT
GGGCAAACTCCAAGATGTGGTGAACCAGAATGCCCAGGCTCTGAACACCCTGGTGAAGCAACTTTCCA
GCAACTTTGGAGCCATCTCCTCTGTGCTGAATGACATCCTGAGCAGACTGGACAAGGTGGAGGCTGAG
GTCCAGATTGACAGACTGATTACAGGCAGACTCCAATCCCTCCAAACCTATGTGACCCAACAACTTAT
CAGGGCTGCTGAGATTAGGGCATCTGCCAACCTGGCTGCCACCAAGATGAGTGAGTGTGTGCTGGGAC
AAAGCAAGAGGGTGGACTTCTGTGGCAAGGGCTACCACCTGATGAGTTTTCCACAGTCTGCCCCTCAT
GGAGTGGTGTTCCTGCATGTGACCTATGTGCCTGCCCAGGAGAAGAACTTCACCACAGCCCCTGCCAT
CTGCCATGATGGCAAGGCTCACTTTCCAAGGGAGGGAGTGTTTGTGAGCAATGGCACCCACTGGTTTG
TGACCCAGAGGAACTTCTATGAACCACAGATTATCACCACAGACAACACCTTTGTGTCTGGCAACTGT
GATGTGGTGATTGGCATTGTGAACAACACAGTCTATGACCCACTCCAACCTGAACTGGACTCCTTCAA
GGAGGAACTGGACAAATACTTCAAGAACCACACCAGCCCTGATGTGGACCTGGGAGACATCTCTGGCA
TCAATGCCTCTGTGGTGAACATCCAGAAGGAGATTGACAGACTGAATGAGGTGGCTAAGAACCTGAAT
GAGTCCCTGATTGACCTCCAAGAACTGGGCAAATATGAACAATACATCAAGTGGCCATGGTACATCTG
GCTGGGCTTCATTGCTGGACTGATTGCCATTGTGATGGTGACCATAATGCTGTGTTGTATGACCTCCT
GTTGTTCCTGTCTGAAAGGCTGTTGTTCCTGTGGCTCCTGTTGTAAGTTTGATGAGGATGACTCTGAA
CCTGTGCTGAAAGGAGTGAAACTGCACTACACCggcagtggagagggcagaggaagtctgctaacatg
cggtgacgtcgaggagaatcctggcccaATGGCCGACTCAAATGGGACCATTACTGTAGAGGAGCTGA
AAAAACTGTTGGAGCAATGGAACCTTGTGATTGGATTTCTCTTCTTGACATGGATTTGTCTCCTGCAA
TTCGCATACGCGAATCGCAACCGATTCCTCTATATTATAAAACTTATATTCCTTTGGCTCCTGTGGCC
TGTGACCCTTGCCTGCTTCGTACTCGCGGCGGTGTATCGAATAAACTGGATAACTGGCGGCATAGCAA
TAGCTATGGCATGCCTGGTAGGTTTGATGTGGCTCTCCTATTTTATAGCGTCATTCAGACTTTTTGCA
CGCACGCGAAGCATGTGGTCATTTAACCCTGAAACAAATATCTTGCTTAATGTCCCCCTGCATGGCAC
AATTTTGACCCGACCGCTCCTTGAATCAGAATTGGTGATTGGAGCCGTGATCCTGAGGGGCCACTTGA
GGATCGCAGGACATCATCTTGGTCGCTGTGATATTAAGGATTTGCCAAAAGAAATAACTGTAGCTACC
AGTCGAACTTTGAGCTATTACAAGCTTGGAGCGTCCCAAAGGGTCGCTGGCGATAGTGGATTTGCAGC
CTATTCACGCTACAGGATAGGGAACTATAAACTGAATACAGACCACTCATCATCTTCTGATAACATTG
CTTTGCTCGTCCAGTGAgaattcAATAAAAGATCTTTATTTTCATTAGATCTGTGTGTTGGTTTTTTG
TGTgaccgagcggccgcaggaacccctagtgatggagttggccactccctctctgcgcgctcgctcgc
tcactgaggccgggcgaccaaaggtcgcccgacgcccgggctttgcccgggcggcctcagtgagcgag
cgagcgcgcagctgcctgcaggggcgcctgatgcggtattttctccttacgcatctgtgcggtatttc
acaccgcatacgtcaaagcaaccatagtacgcgccctgtagcggcgcattaagcgcggcgggtgtggt
ggttacgcgcagcgtgaccgctacacttgccagcgccctagcgcccgctcctttcgctttcttccctt
cctttctcgccacgttcgccggctttccccgtcaagctctaaatcgggggctccctttagggttccga
tttagtgctttacggcacctcgaccccaaaaaacttgatttgggtgatggttcacgtagtgggccatc
gccctgatagacggtttttcgccctttgacgttggagtccacgttctttaatagtggactcttgttcc
aaactggaacaacactcaaccctatctcgggctattcttttgatttataagggattttgccgatttcg
gcctattggttaaaaaatgagctgatttaacaaaaatttaacgcgaattttaacaaaatattaacgtt
tacaattttatggtgcactctcagtacaatctgctctgatgccgcatagttaagccagccccgacacc
cgccaacacccgctgacgcgccctgacgggcttgtctgctcccggcatccgcttacagacaagctgtg
accgtctccgggagctgcatgtgtcagaggttttcaccgtcatcaccgaaacgcgcgagacgaaaggg
cctcgtgatacgcctatttttataggttaatgtcatgataataatggtttcttagacgtcaggtggca
cttttcggggaaatgtgcgcggaacccctatttgtttatttttctaaatacattcaaatatgtatccg
ctcatgagacaataaccctgataaatgcttcaataatattgaaaaaggaagagtatgagtattcaaca
tttccgtgtcgcccttattcccttttttgcggcattttgccttcctgtttttgctcacccagaaacgc
tggtgaaagtaaaagatgctgaagatcagttgggtgcacgagtgggttacatcgaactggatctcaac
agcggtaagatccttgagagttttcgccccgaagaacgttttccaatgatgagcacttttaaagttct
gctatgtggcgcggtattatcccgtattgacgccgggcaagagcaactcggtcgccgcatacactatt
ctcagaatgacttggttgagtactcaccagtcacagaaaagcatcttacggatggcatgacagtaaga
gaattatgcagtgctgccataaccatgagtgataacactgcggccaacttacttctgacaacgatcgg
aggaccgaaggagctaaccgcttttttgcacaacatgggggatcatgtaactcgccttgatcgttggg
aaccggagctgaatgaagccataccaaacgacgagcgtgacaccacgatgcctgtagcaatggcaaca
acgttgcgcaaactattaactggcgaactacttactctagcttcccggcaacaattaatagactggat
ggaggcggataaagttgcaggaccacttctgcgctcggcccttccggctggctggtttattgctgata
aatctggagccggtgagcgtgggtctcgcggtatcattgcagcactggggccagatggtaagccctcc
cgtatcgtagttatctacacgacggggagtcaggcaactatggatgaacgaaatagacagatcgctga
gataggtgcctcactgattaagcattggtaactgtcagaccaagtttactcatatatactttagattg
atttaaaacttcatttttaatttaaaaggatctaggtgaagatcctttttgataatctcatgaccaaa
atcccttaacgtgagttttcgttccactgagcgtcagaccccgtagaaaagatcaaaggatcttcttg
agatcctttttttctgcgcgtaatctgctgcttgcaaacaaaaaaaccaccgctaccagcggtggttt
gtttgccggatcaagagctaccaactctttttccgaaggtaactggcttcagcagagcgcagatacca
aatactgtccttctagtgtagccgtagttaggccaccacttcaagaactctgtagcaccgcctacata
cctcgctctgctaatcctgttaccagtggctgctgccagtggcgataagtcgtgtcttaccgggttgg
actcaagacgatagttaccggataaggcgcagcggtcgggctgaacggggggttcgtgcacacagccc
agcttggagcgaacgacctacaccgaactgagatacctacagcgtgagctatgagaaagcgccacgct
tcccgaagggagaaaggcggacaggtatccggtaagcggcagggtcggaacaggagagcgcacgaggg
agcttccagggggaaacgcctggtatctttatagtcctgtcgggtttcgccacctctgacttgagcgt
cgatttttgtgatgctcgtcaggggggcggagcctatggaaaaacgccagcaacgcggcctttttacg
gttcctggccttttgctggccttttgctcacatgt
pMD145-pMSCV-EFS-N protein-TA-mScarlet
SEQ ID NO: 122
gaccccacctgtaggtttggcaagctagcttaagtaacgccattttgcaaggcatggaaaatacataa
ctgagaatagagaagttcagatcaaggttaggaacagagagacagcagaatatgggccaaacaggata
tctgtggtaagcagttcctgccccggctcagggccaagaacagatggtccccagatgcggtcccgccc
tcagcagtttctagagaaccatcagatgtttccagggtgccccaaggacctgaaatgaccctgtgcct
tatttgaactaaccaatcagttcgcttctcgcttctgttcgcgcgcttctgctccccgagctcaataa
aagagcccacaacccctcactcggcgcgccagtcctccgatagactgcgtcgcccgggtacccgtatt
cccaataaagcctcttgctgtttgcatccgaatcgtggactcgctgatccttgggagggtctcctcag
attgattgactgcccacctcgggggtctttcatttggaggttccaccgagatttggagacccctgccc
agggaccaccgacccccccgccgggaggtaagctggccagcggtcgtttcgtgtctgtctctgtcttt
gtgcgtgtttgtgccggcatctaatgtttgcgcctgcgtctgtactagttagctaactagctctgtat
ctggcggacccgtggtggaactgacgagttctgaacacccggccgcaaccctgggagacgtcccaggg
actttgggggccgtttttgtggcccgacctgaggaagggagtcgatgtggaatccgaccccgtcagga
tatgtggttctggtaggagacgagaacctaaaacagttcccgcctccgtctgaatttttgctttcggt
ttggaaccgaagccgcgcgtcttgtctgctgcagcgctgcagcatcgttctgtgttgtctctgtctga
ctgtgtttctgtatttgtctgaaaattagggccagactgttaccactcccttaagtttgaccttaggt
cactggaaagatgtcgagcggatcgctcacaaccagtcggtagatgtcaagaagagacgttgggttac
cttctgctctgcagaatggccaacctttaacgtcggatggccgcgagacggcacctttaaccgagacc
tcatcacccaggttaagatcaaggtcttttcacctggcccgcatggacacccagaccaggtcccctac
atcgtgacctgggaagccttggcttttgacccccctccctgggtcaagccctttgtacaccctaagcc
tccgcctcctcttcctccatccgccccgtctctcccccttgaacctcctcgttcgaccccgcctcgat
cctccctttatccagccctcactccttctctaggcgccggaattagatctctcgagatcgatgccttg
agacaaatggcagtattcatccacaattttaaaagaaaaggggggattggggggtacagtgcagggga
aagaatagtagacataatagcaacagacatacaaactaaagaattacaaaaacaaattacaaaaattc
aaaattttcgggtttattacagggacagcagagatccactttggcgccggctcgagggggccGCTCCG
GTGCCCGTCAGTGGGCAGAGCGCACATCGCCCACAGTCCCCGAGAAGTTGGGGGGAGGGGTCGGCAAT
TGAACCGGTGCCTAGAGAAGGTGGCGCGGGGTAAACTGGGAAAGTGATGTCGTGTACTGGCTCCGCCT
TTTTCCCGAGGGTGGGGGAGAACCGTATATAAGTGCAGTAGTCGCCGTGAACGTTCTTTTTCGCAACG
GGTTTGCCGCCAGAACACAGGTAAGTGCCGTGTGTGGTTCCCGCGGGCCTGGCCTCTTTACGGGTTAT
GGCCCTTGCGTGCCTTGAATTACTTCCACGCCCCTGGCTGCAGTACGTGATTCTTGATCCCGAGCTTC
GGGTTGGAAGTGGGTGGGAGAGTTCGAGGCCTTGCGCTTAAGGAGCCCCTTCGCCTCGTGCTTGAGTT
GAGGCCTGGCTTGGGCGCTGGGGCCGCCGCGTGCGAATCTGGTGGCACCTTCGCGCCTGTCTCGCTGC
TTTCGATAAGTCTCTAGCCATTTAAAATTTTTGATGACCTGCTGCGACGCTTTTTTTCTGGCAAGATA
GTCTTGTAAATGCGGGCCAAGATCTGCACACTGGTATTTCGGTTTTTGGGGCCGCGGGCGGCGACGGG
GCCCGTGCGTCCCAGCGCACATGTTCGGCGAGGCGGGGCCTGCGAGCGCGGCCACCGAGAATCGGACG
GGGGTAGTCTCAAGCTGGCCGGCCTGCTCTGGTGCCTGGCCTCGCGCCGCCGTGTATCGCCCCGCCCT
GGGCGGCAAGGCTGGCCCGGTCGGCACCAGTTGCGTGAGCGGAAAGATGGCCGCTTCCCGGCCCTGCT
GCAGGGAGCTCAAAATGGAGGACGCGGCGCTCGGGAGAGCGGGCGGGTGAGTCACCCACACAAAGGAA
AAGGGCCTTTCCGTCCTCAGCCGTCGCTTCATGTGACTCCACGGAGTACCGGGCGCCGTCCAGGCACC
TCGATTAGTTCTCGAGCTTTTGGAGTACGTCGTCTTTAGGTTGGGGGGAGGGGTTTTATGCGATGGAG
TTTCCCCACACTGAGTGGGTGGAGACTGAAGTTAGGCCAGCTTGGCACTTGATGTAATTCTCCTTGGA
ATTTGCCCTTTTTGAGTTTGGATCTTGGTTCATTCTCAAGCCTCAGACAGTGGTTCAAAGTTTTTTTC
TTCCATTTCAGGTGTCGTGAGGATCTATTTCCGGTGAATTCGCCGCCACCATGAGCGATAACGGCCCC
CAAAACCAACGGAATGCCCCTCGGATAACATTTGGTGGCCCTAGTGATTCCACAGGAAGTAACCAAAA
CGGAGAACGGTCTGGAGCCCGCTCTAAACAGAGGCGACCACAGGGATTGCCAAATAACACAGCGTCAT
GGTTTACAGCCCTGACTCAACATGGAAAGGAGGACTTGAAATTTCCACGAGGACAGGGTGTACCAATA
AACACAAACAGTAGTCCAGACGATCAGATCGGGTATTATAGAAGGGCCACGAGACGCATCAGAGGTGG
AGATGGTAAAATGAAAGATCTCAGTCCGCGCTGGTACTTTTATTATTTGGGAACCGGCCCAGAAGCGG
GCCTCCCATATGGAGCCAATAAGGACGGGATTATATGGGTGGCTACAGAGGGAGCCCTCAATACGCCA
AAAGATCACATCGGAACAAGAAACCCTGCTAATAATGCCGCAATCGTGCTGCAGTTGCCTCAGGGGAC
GACGCTTCCTAAAGGCTTTTACGCAGAAGGATCACGCGGCGGCAGCCAAGCATCCTCAAGGTCTAGTT
CCAGAAGTCGAAACAGCTCCAGAAACTCCACACCAGGGTCCAGTAGGGGCACAAGTCCGGCGCGGATG
GCGGGCAACGGCGGAGACGCCGCACTCGCTCTGTTGCTCCTGGACCGGCTCAACCAACTTGAATCCAA
GATGAGTGGTAAGGGACAGCAGCAACAAGGTCAAACCGTAACCAAGAAAAGCGCTGCAGAAGCTTCTA
AAAAACCTCGACAAAAACGGACCGCTACGAAGGCATATAACGTTACTCAAGCCTTCGGGAGACGAGGG
CCGGAGCAAACCCAGGGGAATTTTGGAGATCAGGAGCTCATCCGGCAAGGGACAGATTACAAACATTG
GCCTCAAATCGCTCAGTTCGCGCCGTCCGCAAGTGCTTTTTTCGGAATGTCTCGCATCGGTATGGAAG
TAACACCATCCGGGACATGGCTCACTTATACCGGCGCCATCAAGCTGGACGACAAAGATCCAAATTTC
AAAGATCAGGTGATTCTCTTGAATAAACACATAGACGCGTATAAGACGTTTCCACCCACCGAACCCAA
GAAGGACAAAAAGAAAAAAGCTGATGAAACACAAGCGTTGCCCCAGCGACAGAAGAAGCAGCAAACCG
TCACCCTCCTGCCAGCCGCTGATCTCGATGACTTCTCCAAGCAACTTCAACAGTCCATGAGCTCAGCC
GATTCTACTCAGGCGggatccggcgcaacaaacttctctctgctgaaacaagccggagatgtcgaaga
gaatcctggaccgatgagtaaaggagaagctgtgattaaagagttcatgcgcttcaaagttcacatgg
agggttctatgaacggtcacgagttcgagatcgaaggcgaaggcgagggccgtccgtatgaaggcacc
cagaccgccaaactgaaagtgactaaaggcggcccgctgcctttttcctgggacatcctgagcccgca
atttatgtacggttctagggcgttcaccaaacacccagcggatatcccggactattataagcagtctt
ttccggaaggtttcaagtgggaacgcgtaatgaattttgaagatggtggtgccgtgaccgtcactcag
gacacctccctggaggatggcaccctgatctataaagttaaactgcgtggtactaattttccacctga
tggcccggtgatgcagaaaaagacgatgggttgggaggcgtctaccgaacgcttgtatccggaagatg
gtgtgctgaaaggcgacattaaaatggccctgcgcctgaaagatggcggccgctatctggctgacttc
aaaaccacgtacaaagccaagaaacctgtgcagatgcctggcgcgtacaatgtggaccgcaaactgga
catcacctctcataatgaagattatacggtggtagagcaatatgagcgctccgagggtcgtcattcta
ccggtggcatggatgaactatacaaataagaattcagcgctaataaaagatctttattttcattagat
ctgtgtgttggttttttgtgtcgataaaataaaagattttatttagtctccagaaaaaggggggaatg
aaagaccccacctgtaggtttggcaagctagcttaagtaacgccattttgcaaggcatggaaaataca
taactgagaatagagaagttcagatcaaggttaggaacagagagacagcagaatatgggccaaacagg
atatctgtggtaagcagttcctgccccggctcagggccaagaacagatggtccccagatgcggtcccg
ccctcagcagtttctagagaaccatcagatgtttccagggtgccccaaggacctgaaatgaccctgtg
ccttatttgaactaaccaatcagttcgcttctcgcttctgttcgcgcgcttctgctccccgagctcaa
taaaagagcccacaacccctcactcggcgcgccagtcctccgatagactgcgtcgcccgggtacccgt
gtatccaataaaccctcttgcagttgcatccgacttgtggtctcgctgttccttgggagggtctcctc
tgagtgattgactacccgtcagcgggggtctttcatgggtaacagtttcttgaagttggagaacaaca
ttctgagggtaggagtcgaatattaagtaatcctgactcaattagccactgttttgaatccacatact
ccaatactcctgaaatagttcattatggacagcgcagaaagagctggggagaattgtgaaattgttat
ccgctcacaattccacacaacatacgagccggaagcataaagtgtaaagcctggggtgcctaatgagt
gagctaactcacattaattgcgttgcgctcactgcccgctttccagtcgggaaacctgtcgtgccagc
tgcattaatgaatcggccaacgcgcggggagaggcggtttgcgtattgggcgctcttccgcttcctcg
ctcactgactcgctgcgctcggtcgttcggctgcggcgagcggtatcagctcactcaaaggcggtaat
acggttatccacagaatcaggggataacgcaggaaagaacatgtgagcaaaaggccagcaaaaggcca
ggaaccgtaaaaaggccgcgttgctggcgtttttccataggctccgcccccctgacgagcatcacaaa
aatcgacgctcaagtcagaggtggcgaaacccgacaggactataaagataccaggcgtttccccctgg
aagctccctcgtgcgctctcctgttccgaccctgccgcttaccggatacctgtccgcctttctccctt
cgggaagcgtggcgctttctcatagctcacgctgtaggtatctcagttcggtgtaggtcgttcgctcc
aagctgggctgtgtgcacgaaccccccgttcagcccgaccgctgcgccttatccggtaactatcgtct
tgagtccaacccggtaagacacgacttatcgccactggcagcagccactggtaacaggattagcagag
cgaggtatgtaggcggtgctacagagttcttgaagtggtggcctaactacggctacactagaagaaca
gtatttggtatctgcgctctgctgaagccagttaccttcggaaaaagagttggtagctcttgatccgg
caaacaaaccaccgctggtagcggtggtttttttgtttgcaagcagcagattacgcgcagaaaaaaag
gatctcaagaagatcctttgatcttttctacggggtctgacgctcagtggaacgaaaactcacgttaa
gggattttggtcatgagattatcaaaaaggatcttcacctagatccttttaaattaaaaatgaagttt
taaatcaatctaaagtatatatgagtaaacttggtctgacagttaccaatgcttaatcagtgaggcac
ctatctcagcgatctgtctatttcgttcatccatagttgcctgactccccgtcgtgtagataactacg
atacgggagggcttaccatctggccccagtgctgcaatgataccgcgagacccacgctcaccggctcc
agatttatcagcaataaaccagccagccggaagggccgagcgcagaagtggtcctgcaactttatccg
cctccatccagtctattaattgttgccgggaagctagagtaagtagttcgccagttaatagtttgcgc
aacgttgttgccattgctacaggcatcgtggtgtcacgctcgtcgtttggtatggcttcattcagctc
cggttcccaacgatcaaggcgagttacatgatcccccatgttgtgcaaaaaagcggttagctccttcg
gtcctccgatcgttgtcagaagtaagttggccgcagtgttatcactcatggttatggcagcactgcat
aattctcttactgtcatgccatccgtaagatgcttttctgtgactggtgagtactcaaccaagtcatt
ctgagaatagtgtatgcggcgaccgagttgctcttgcccggcgtcaatacgggataataccgcgccac
atagcagaactttaaaagtgctcatcattggaaaacgttcttcggggcgaaaactctcaaggatctta
ccgctgttgagatccagttcgatgtaacccactcgtgcacccaactgatcttcagcatcttttacttt
caccagcgtttctgggtgagcaaaaacaggaaggcaaaatgccgcaaaaaagggaataagggcgacac
ggaaatgttgaatactcatactcttcctttttcaatattattgaagcatttatcagggttattgtctc
atgagcggatacatatttgaatgtatttagaaaaataaacaaataggggttccgcgcacatttccccg
aaaagtgccacctgacgtctaagaaaccattattatcatgacattaacctataaaaataggcgtatca
cgaggccctttcgtctcgcgcgtttcggtgatgacggtgaaaacctctgacacatgcagctcccggag
acggtcacagcttgtctgtaagcggatgccgggagcagacaagcccgtcagggcgcgtcagcgggtgt
tggcgggtgtcggggctggcttaactatgcggcatcagagcagattgtactgagagtgcaccatatgc
ggtgtgaaataccgcacagatgcgtaaggagaaaataccgcatcaggcgccattcgccattcaggctg
cgcaactgttgggaagggcgatcggtgcgggcctcttcgctattacgccagctggcgaaagggggatg
tgctgcaaggcgattaagttgggtaacgccagggttttcccagtcacgacgttgtaaaacgacggcgc
aaggaatggtgcatgcaaggagatggcgcccaacagtcccccggccacggggcctgccaccataccca
cgccgaaacaagcgctcatgagcccgaagtggcgagcccgatcttccccatcggtgatgtcggcgata
taggcgccagcaaccgcacctgtggcgccggtgatgccggccacgatgcgtccggcgtagaggcgatt
agtccaatttgttaaagacaggatatcagtggtccaggctctagttttgactcaacaatatcaccagc
tgaagcctatagagtacgagccatagataaaataaaagattttatttagtctccagaaaaagggggga
atgaaa

### Enumerated Embodiments

The following enumerated embodiments are provided, the numbering of which is not to be construed as designating levels of importance.

Embodiment 1 provides an isolated messenger ribonucleic acid (mRNA) comprising a 5′ untranslated region (UTR), a 3′ UTR, and an open reading frame encoding a spike protein sequence, wherein the spike protein sequence comprises all or a portion of a coronavirus spike protein, further wherein the spike protein sequence comprises one or more mutations that stabilize the spike protein in a prefusion conformation.

Embodiment 2 provides the isolated mRNA of Embodiment 1, wherein the coronavirus is a variant of a coronavirus selected from the group consisting of SARS-CoV-2, MERS, and SARS-CoV.

Embodiment 3 provides the isolated mRNA of Embodiment 2, wherein the variant is selected from SARS-CoV-2 B.1.1.7 (Alpha variant), SARS-CoV-2 B.1.351 (Beta variant), SARS-CoV-2 B.1.617, SARS-CoV-2 B.1.617.1 (Kappa variant), SARS-CoV-2 B.1.617.2 (Delta variant), SARS-CoV-2 B.1.617.3, SARS-CoV-2 B.1.1.529/BA.1 (Omicron variant), SARS-CoV-2 BA.5, SARS-CoV-2 BA.2, SARS-CoV-2 BA.2.12.1, and SARS-CoV-2 BA.4/5.

Embodiment 4 provides the isolated mRNA of any one of Embodiments 1-3, wherein the spike protein sequence comprises all or a portion of the S2 subunit of the spike protein, wherein the one or more mutations comprise one or more proline substitutions in the S2 subunit.

Embodiment 5 provides the isolated mRNA of Embodiment 4, wherein the one or more proline substitutions are selected from the group consisting of F817P, A892P, A899P, A942P, K986P, V987P, and combinations thereof, wherein the amino acid positions of the proline substitutions are relative to the native SARS-CoV-2 S sequence set forth in SEQ ID NO: 2.

Embodiment 6 provides the isolated mRNA of any one of Embodiments 1-5, wherein the spike protein sequence further comprises an S1/S2 protease cleavage site of the spike protein, wherein the cleavage site comprises one or more mutations to inhibit protease cleavage of the spike protein.

Embodiment 7 provides the isolated mRNA of Embodiment 6, wherein the cleavage site is a furin cleavage site, optionally wherein the furin cleavage site is deleted or replaced with the sequence GSAS (SEQ ID NO:11).

Embodiment 8 provides the isolated mRNA of any one of Embodiments 1-7, wherein the spike protein sequence comprises the amino acid sequence of any one of SEQ ID NOs: 2-10, 34, 46-54, and 57-60, and/or an amino acid sequence having 75%, 80%, 85%, 90%, 91%, 92%, 93%, 94%, 95%, 96%, 97%, 98%, 99%, 99.5% or more sequence identity to any one of SEQ ID NOs: 2-10, 34, 46-54, and 57-60.

Embodiment 9 provides an isolated, chimeric messenger ribonucleic acid (mRNA) comprising a 5′ UTR, a 3′ UTR, and two or more open reading frames, wherein each open reading frame encodes a different spike protein sequence, wherein each spike protein sequence comprises a spike protein subunit from the spike protein of a distinct coronavirus species or variant thereof.

Embodiment 10 provides the isolated mRNA of Embodiment 9, wherein the two or more open reading frames comprise a first open reading frame and a second open reading frame, wherein the first open reading frame encodes an S1 subunit of a first coronavirus selected from SARS-CoV, MERS-CoV, and SARS-CoV-2, and wherein the second open reading frame encodes an S2 subunit of a second coronavirus selected from SARS-CoV, MERS-CoV, and SARS-CoV-2.

Embodiment 11 provides the isolated mRNA of Embodiment 10, wherein at least one applies:
(a) the S1 subunit is from SARS-CoV-2 B.1.351 and the S2 subunit is from SARS-CoV-2 B.1.617, such as but not limited to B.1.617.2;
(b) the S1 subunit is from SARS-CoV and the S2 subunit is from SARS-CoV-2 B.1.617, such as but not limited to B.1.617.2; or
(c) the S1 subunit is from MERS-CoV and the S2 subunit is from SARS-CoV-2 B.1.617, such as but not limited to B.1.617.2.

Embodiment 12 provides the isolated mRNA of Embodiment 10 or 11, wherein the S2 subunit comprises one or more mutations that stabilize the spike protein in a prefusion conformation, optionally wherein the one or more mutations are selected from the group consisting of F817P, A892P, A899P, A942P, K986P, V987P, and combinations thereof.

Embodiment 13 provides the isolated mRNA of any one of Embodiments 10-12, wherein there is no linker or other domain intervening between the first and second open reading frames.

Embodiment 14 provides the isolated mRNA of Embodiment 9, wherein each open reading frame further comprises a sequence encoding a SPY tag, wherein the SPY tag is positioned at the C-terminus of the spike protein subunit.

Embodiment 15 provides the isolated mRNA of Embodiment 14, further comprising a sequence encoding a 2A self-cleaving peptide between adjacent open reading frames.

Embodiment 16 provides the isolated mRNA of Embodiment 15, wherein the two or more open reading frames comprise a first open reading frame, a second open reading frame, and optionally a third open reading frame, wherein:
(a) the first open reading frame encodes an S1 subunit of a SARS-CoV-2 variant, in certain non-limiting embodiments SARS-CoV-2 B.1.351;
(b) the second open reading frame encodes an S1 subunit of SARS-CoV; and
(c) the third open reading frame encodes an S1 subunit of MERS-CoV.

Embodiment 17 provides an isolated messenger ribonucleic acid (mRNA) comprising a 5′ UTR, a 3′ UTR, and an open reading frame, wherein the open readying frame encodes an S2 subunit of a coronavirus spike protein and a SPY catcher, wherein the SPY catcher is positioned at the N-terminus of the spike protein S2 subunit, optionally wherein the coronavirus is selected from SARS-CoV, MERS-CoV, SARS-CoV-2, and variants thereof.

Embodiment 18 provides the isolated mRNA of any one of Embodiments 1-17, wherein the mRNA further comprises a 5′ cap, a poly(A) tail, one or more modified nucleotides, one or more structural modifications, or a combination thereof.

Embodiment 19 provides the isolated mRNA of Embodiment 18, wherein the one or more modified nucleotides are independently selected from pseudouridine, N1-methyl-pseudouridine, N1-Methylpseudouridine-5′-Triphosphate-(N-1081), 1-ethylpseudouridine, 2-thiouridine, 4′-thiouridine, 5-methoxyuridine, 5-methoxyuridine, N6-methyladenosine, and 5-methylcytosine.

Embodiment 20 provides the isolated mRNA of Embodiment 18 or 19, wherein the 5′ cap is cap0, cap1, cap 2, ARCA, beta-S-ARCA, m7G, inosine, N1-methyl-guanosine, 2′-fluoro-guanosine, 7-deaza-guanosine, 8-oxo-guanosine, 2-amino-guanosine, LNA-guanosine, tri-methylgranosine (TMG), nicotinamide adenine dinucleotide (NAD), cap AG, cap AU, cap GG, or 2-azido-guanosine.

Embodiment 21 provides the isolated mRNA of any one of Embodiments 1-20, wherein the mRNA is codon optimized for expression in a eukaryotic cell.

Embodiment 22 provides the isolated mRNA of any one of Embodiments 1-21, wherein the mRNA is produced by in vitro transcription.

Embodiment 23 provides an isolated polynucleotide encoding the mRNA of any one of Embodiments 1-22, optionally wherein the polynucleotide comprises one or more promoters and/or a polyadenylation signal operably linked to a sequence encoding the mRNA.

Embodiment 24 provides a vector comprising the polynucleotide of Embodiment 23.

Embodiment 25 provides the vector of Embodiment 24, wherein the vector is a viral vector, optionally an adeno-associated virus (AAV) vector, optionally AAV9.

Embodiment 26 provides a method of producing a recombinant coronavirus spike protein stabilized in a prefusion conformation, the method comprising introducing the polynucleotide of Embodiment 23 or vector of Embodiment 24 or 25 to a host cell and incubating the host cell under conditions sufficient for expression of the polynucleotide, thereby producing the recombinant spike protein, wherein the polynucleotide encodes the mRNA of any one of Embodiments 1-8.

Embodiment 27 provides a method of producing a chimeric/hybrid coronavirus spike protein, the method comprising introducing the polynucleotide of Embodiment 23 or vector of Embodiment 24 or 25 to a host cell and incubating the host cell under conditions sufficient for expression of the polynucleotide, thereby producing the chimeric/hybrid spike protein, wherein the polynucleotide encodes the mRNA of any one of Embodiments 9-17.

Embodiment 28 provides the method of Embodiment 26 or 27, further comprising purifying the spike protein from the cell.

Embodiment 29 provides a virus-like particle comprising the protein encoded by the mRNA of any one of Embodiments 1-22.

Embodiment 30 provides a lipid nanoparticle comprising the mRNA of any one of Embodiments 1-22.

Embodiment 31 provides a lipid nanoparticle comprising two or more distinct mRNAs, wherein each mRNA comprises an open reading frame encoding all or a portion of a coronavirus spike protein derived from a distinct coronavirus species or variant thereof.

Embodiment 32 provides the lipid nanoparticle of Embodiment 31 comprising three mRNAs, wherein the spike protein or portion thereof is selected from MERS-CoV, SARS-CoV, SARS-CoV-2, and variants thereof.

Embodiment 33 provides the lipid nanoparticle of any one of Embodiments 30-32, wherein the molar ratio of lipid to mRNA is in the range of about 5:1 to 20:1, preferably 6:1.

Embodiment 34 provides the lipid nanoparticle of any one of Embodiments 30-33, wherein the lipid nanoparticle comprises at least one ionizable cationic lipid, at least one helper lipid, at least one sterol, and at least one PEG-modified lipid.

Embodiment 35 provides the lipid nanoparticle of Embodiment 34, wherein the at least one ionizable cationic lipid comprises 1,2-dimyristoyl-sn-glycero-3-ethylphosphocholine (DMEPC), 1,2-di-O-octadecenyl-3-trimethylammonium propane (DOTMA), 1,2-dioleoyl-3-trimethylammonium propane (DOTAP), PNI ionizable lipid, SM-102, DLin-MC3-DMA, DLin-KC2-DMA, ALC-0315, or a combination thereof.

Embodiment 36 provides the lipid nanoparticle of Embodiment 34 or 35, wherein the at least one helper lipid comprises 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3-phosphocholin (POPC), 1,2-dioleoyl-sn-glycero-3-phosphocholine (DOPC), or a combination thereof.

Embodiment 37 provides the lipid nanoparticle of any one of Embodiments 34-36, wherein the at least one PEG-modified lipid comprises 1,2-dimyristoyl-racglycero-3-methoxypolyethylene glycol-2000 (PEG-DMG), 1,2-Distearoyl-sn-glycerol, methoxypolyethylene glycol (PEG-DSG), 1,2-Dipalmitoyl-sn-glycerol, methoxypolyethylene glycol (PEG-DPG), mPEG-OH, mPEG-AA (mPEG-CM), mPEG-CH₂CH₂CH₂—NH₂, MPEG-DMG, mPEG-N,N-Ditetradecylacetamide (ALC-0159), mPEG-DSPE, mPEG-DPPE, or a combination thereof.

Embodiment 38 provides the lipid nanoparticle of any one of Embodiments 34-37, wherein the at least one sterol is cholesterol.

Embodiment 39 provides the lipid nanoparticle of any one of Embodiments 34-38, wherein the lipid nanoparticle comprises about 20-60% ionizable cationic lipid, about 5-25% helper lipid, about 25-55% sterol, and about 0.5-15% PEG-modified lipid.

Embodiment 40 provides a pharmaceutical composition comprising the lipid nanoparticle of any one of Embodiments 30-39 and a pharmaceutically acceptable carrier or excipient.

Embodiment 41 provides a vaccine comprising one or more lipid nanoparticles of any one of Embodiments 30-39 or the pharmaceutical composition of Embodiment 40, and further comprising a pharmaceutically acceptable adjuvant.

Embodiment 42 provides a method of inducing in a subject an immune response to a coronavirus, comprising administering to the subject the vaccine of Embodiment 41 in an amount effective to generate the immune response.

Embodiment 43 provides the method of Embodiment 42, wherein the immune response comprises a T cell response and/or a B cell response.

Embodiment 44 provides the method of Embodiment 43, wherein the immune response comprises a neutralizing antibody response specific to the coronavirus spike protein.

Embodiment 45 provides the method of any one of Embodiments 42-44, wherein the immune response inhibits infection by the coronavirus and/or replication of the coronavirus in the subject.

Embodiment 46 provides the method of any one of Embodiments 42-45, wherein the subject is administered a single dose of the vaccine.

Embodiment 47 provides the method of any one of Embodiments 42-45, wherein the subject is administered two or more doses of the vaccine, optionally wherein the two or more doses are administered 14-28 days apart.

Embodiment 48 provides the method of Embodiment 47, wherein each administration of the vaccine comprises a dose of about 1 μg, 3 μg, 10 μg, 25 μg, 30 μg or 100 μg.

Embodiment 49 provides the method of any one of Embodiments 42-48, wherein the effective amount is a total dose of about 1-500 μg, inclusive.

Embodiment 50 provides the method of any one of Embodiments 42-49, wherein the vaccine is administered by intradermal injection, intramuscular injection, oral administration, intranasal administration, or intratracheal administration.

Embodiment 51 provides the method of any one of Embodiments 42-50, wherein the subject has been exposed to, is infected with, or is at risk of infection by the coronavirus.

Embodiment 52 provides the method of any one of Embodiments 42-51, wherein the subject is immunocompromised.

Embodiment 53 provides the method of any one of Embodiments 42-52, wherein the subject is human.

Embodiment 54 provides the method of any one of Embodiments 42-53, wherein the coronavirus is selected from MERS-CoV, SARS-CoV, SARS-CoV-2, and variants thereof.

### OTHER EMBODIMENTS

It is understood that the disclosed method and compositions are not limited to the particular methodology, protocols, and reagents described as these may vary. It is also to be understood that the terminology used herein is for the purpose of describing particular embodiments only, and is not intended to limit the scope of the present disclosure which will be limited only by the appended claims.

Those skilled in the art will recognize, or be able to ascertain using no more than routine experimentation, many equivalents to the specific embodiments of the method and compositions described herein. Such equivalents are intended to be encompassed by the following claims.

1. An isolated messenger ribonucleic acid (mRNA) comprising a 5′ untranslated region (UTR), a 3′ UTR, and an open reading frame encoding a spike protein sequence,
wherein the spike protein sequence comprises all or a portion of a coronavirus spike protein,
further wherein the spike protein sequence comprises one or more mutations that stabilize the spike protein in a prefusion conformation.

2. The isolated mRNA of claim 1, wherein the coronavirus is a variant of a coronavirus selected from the group consisting of SARS-CoV-2, MERS, and SARS-CoV,
optionally wherein the variant is selected from SARS-CoV-2 B.1.1.7 (Alpha variant), SARS-CoV-2 B.1.351 (Beta variant), SARS-CoV-2 B.1.617, SARS-CoV-2 B.1.617.1 (Kappa variant), SARS-CoV-2 B.1.617.2 (Delta variant), SARS-CoV-2 B.1.617.3, SARS-CoV-2 B.1.1.529/BA.1 (Omicron variant), SARS-CoV-2 BA.5, SARS-CoV-2 BA.2, SARS-CoV-2 BA.2.12.1, and SARS-CoV-2 BA.4/5.

3. (canceled)

4. The isolated mRNA of claim 1, wherein the spike protein sequence comprises all or a portion of the S2 subunit of the spike protein, wherein the one or more mutations comprise one or more proline substitutions in the S2 subunit,
optionally wherein the one or more proline substitutions are selected from the group consisting of F817P, A892P, A899P, A942P, K986P, V987P, and combinations thereof, wherein the amino acid positions of the proline substitutions are relative to the native SARS-CoV-2 S sequence set forth in SEQ ID NO:2.

5. (canceled)

6. The isolated mRNA of claim 1, wherein the spike protein sequence further comprises an S1/S2 protease cleavage site of the spike protein, wherein the cleavage site comprises one or more mutations to inhibit protease cleavage of the spike protein,
optionally wherein the cleavage site is a furin cleavage site, optionally wherein the furin cleavage site is deleted or replaced with the sequence GSAS (SEQ ID NO:11).

7. (canceled)

8. The isolated mRNA of claim 1, wherein the spike protein sequence comprises the amino acid sequence of any one of SEQ ID NOs: 2-10, 34, 46-54, and 57-60, or an amino acid sequence having 75%, 80%, 85%, 90%, 91%, 92%, 93%, 94%, 95%, 96%, 97%, 98%, 99%, 99.5% or more sequence identity to any one of SEQ ID NOs: 2-10, 34, 46-54, and 57-60.

9. An isolated, chimeric messenger ribonucleic acid (mRNA) comprising a 5′ UTR, a 3′ UTR, and two or more open reading frames,
wherein each open reading frame encodes a different spike protein sequence,
wherein each spike protein sequence comprises a spike protein subunit from the spike protein of a distinct coronavirus species or variant thereof.

10. The isolated mRNA of claim 9, wherein the two or more open reading frames comprise a first open reading frame and a second open reading frame,
wherein the first open reading frame encodes an S1 subunit of a first coronavirus selected from SARS-CoV, MERS-CoV, and SARS-CoV-2, and
wherein the second open reading frame encodes an S2 subunit of a second coronavirus selected from SARS-CoV, MERS-CoV, and SARS-CoV-2,
optionally wherein one of the following applies:
(a) the S1 subunit is from SARS-CoV-2 B.1.351 and
the S2 subunit is from SARS-CoV-2 B.1.617;
(b) the S1 subunit is from SARS-CoV and
the S2 subunit is from SARS-CoV-2 B.1.617; or
the S1 subunit is from MERS-CoV and (c)
the S2 subunit is from SARS-CoV-2 B.1.617.

11. (canceled)

12. The isolated mRNA of claim 10, wherein the S2 subunit comprises one or more mutations that stabilize the spike protein in a prefusion conformation, optionally wherein the one or more mutations are selected from the group consisting of F817P, A892P, A899P, A942P, K986P, V987P, and combinations thereof.

13. The isolated mRNA of claim 10, wherein there is no linker or other domain intervening between the first and second open reading frames.

14. The isolated mRNA of claim 9, wherein each open reading frame further comprises a sequence encoding a SPY tag, wherein the SPY tag is positioned at the C-terminus of the spike protein subunit.

15. The isolated mRNA of claim 14, further comprising a sequence encoding a 2A self-cleaving peptide between adjacent open reading frames,
optionally wherein the two or more open reading frames comprise a first open reading frame, a second open reading frame, and optionally a third open reading frame, wherein:
(a) the first open reading frame encodes an S1 subunit of a SARS-CoV-2 variant optionally SARS-CoV-2 B.1.351;
(b) the second open reading frame encodes an S1 subunit of SARS-CoV; and
(c) the third open reading frame encodes an S1 subunit of MERS-CoV.

16. (canceled)

17. An isolated messenger ribonucleic acid (mRNA) comprising a 5′ UTR, a 3′ UTR, and an open reading frame,
wherein the open readying frame encodes an S2 subunit of a coronavirus spike protein and a SPY catcher,
wherein the SPY catcher is positioned at the N-terminus of the spike protein S2 subunit,
optionally wherein the coronavirus is selected from SARS-CoV, MERS-CoV, SARS-CoV-2, and variants thereof.

18. The isolated mRNA of claim 1, wherein the mRNA further comprises a 5′ cap, a poly(A) tail, one or more modified nucleotides, one or more structural modifications, or a combination thereof,
optionally wherein one of the following applies:
(a) the one or more modified nucleotides are independently selected from pseudouridine, N1-methyl-pseudouridine, N1-Methylpseudouridine-5′-Triphosphate-(N-1081), 1-ethylpseudouridine, 2-thiouridine, 4′-thiouridine, 5-methoxyuridine, 5-methoxyuridine, N6-methyladenosine, and 5-methylcytosine;
(b) the 5′ cap is cap0, cap1, cap 2, ARCA, beta-S-ARCA, m7G, inosine, N1-methyl-guanosine, 2′-fluoro-guanosine, 7-deaza-guanosine, 8-oxo-guanosine, 2-amino-guanosine, LNA-guanosine, tri-methylgranosine (TMG), nicotinamide adenine dinucleotide (NAD), cap AG, cap AU, cap GG, or 2-azido-guanosine.

19-20. (canceled)

21. The isolated mRNA of claim 1, wherein at least one of the following applies:
(a) the mRNA is codon optimized for expression in a eukaryotic cell;
(b) the mRNA is produced by in vitro transcription.

22. (canceled)

23. An isolated polynucleotide encoding the mRNA of claim 1, optionally wherein the polynucleotide comprises one or more promoters and/or a polyadenylation signal operably linked to a sequence encoding the mRNA.

24. A vector comprising the polynucleotide of claim 23, optionally wherein the vector is a viral vector, optionally an adeno-associated virus (AAV) vector.

25. (canceled)

26. A method of producing a recombinant coronavirus spike protein stabilized in a prefusion conformation, the method comprising introducing the polynucleotide of claim 23 to a host cell and incubating the host cell under conditions sufficient for expression of the polynucleotide, thereby producing the recombinant spike protein,
wherein the polynucleotide encodes an isolated messenger ribonucleic acid (mRNA) comprising a 5′ untranslated region (UTR), a 3′ UTR, and an open reading frame encoding a spike protein sequence,
wherein the spike protein sequence comprises all or a portion of a coronavirus spike protein,
further wherein the spike protein sequence comprises one or more mutations that stabilize the spike protein in a prefusion conformation
optionally wherein the polynucleotide is comprised within a vector;
optionally wherein the spike protein is further purified from the cell.

27. A method of producing a chimeric/hybrid coronavirus spike protein, the method comprising introducing the polynucleotide of claim 23 to a host cell and incubating the host cell under conditions sufficient for expression of the polynucleotide, thereby producing the chimeric/hybrid spike protein,
wherein the polynucleotide encodes an isolated, chimeric messenger ribonucleic acid (mRNA) comprising a 5′ UTR, a 3′ UTR, and two or more open reading frames,
wherein each open reading frame encodes a different spike protein sequence,
wherein each spike protein sequence comprises a spike protein subunit from the spike protein of a distinct coronavirus species or variant thereof,
optionally wherein the polynucleotide is comprised within a vector;
optionally wherein the spike protein is further purified from the cell.

28. (canceled)

29. A virus-like particle comprising the protein encoded by the mRNA of claim 1.

30. A lipid nanoparticle comprising the mRNA of claim 1.

31. A lipid nanoparticle comprising two or more distinct mRNAs, wherein each mRNA comprises an open reading frame encoding all or a portion of a coronavirus spike protein derived from a distinct coronavirus species or variant thereof.

32. The lipid nanoparticle of claim 31 comprising three mRNAs, wherein the spike protein or portion thereof is selected from MERS-CoV, SARS-CoV, SARS-CoV-2, and variants thereof.

33. The lipid nanoparticle of claim 30, wherein at least one of the following applies:
(a) the molar ratio of lipid to mRNA is in the range of about 5:1 to 20:1, preferably 6:1;
(b) the lipid nanoparticle comprises at least one ionizable cationic lipid, at least one helper lipid, at least one sterol, and at least one PEG-modified lipid.

34. (canceled)

35. The lipid nanoparticle of claim 33, wherein at least one of the following applies:
(a) the at least one ionizable cationic lipid comprises 1,2-dimyristoyl-sn-glycero-3-ethylphosphocholine (DMEPC), 1,2-di-O-octadecenyl-3-trimethylammonium propane (DOTMA), 1,2-dioleoyl-3-trimethylammonium propane (DOTAP), PNI ionizable lipid, SM-102, DLin-MC3-DMA, DLin-KC2-DMA, ALC-0315, or a combination thereof;
(b) the at least one helper lipid comprises 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3-phosphocholin (POPC), 1,2-dioleoyl-sn-glycero-3-phosphocholine (DOPC), or a combination thereof;
(c) the at least one PEG-modified lipid comprises 1,2-dimyristoyl-racglycero-3-methoxypolyethylene glycol-2000 (PEG-DMG), 1,2-Distearoyl-sn-glycerol, methoxypolyethylene glycol (PEG-DSG), 1,2-Dipalmitoyl-sn-glycerol, methoxypolyethylene glycol (PEG-DPG), mPEG-OH, mPEG-AA (mPEG-CM), mPEG-CH₂CH₂CH₂—NH₂, MPEG-DMG, mPEG-N,N-Ditetradecylacetamide (ALC-0159), mPEG-DSPE, mPEG-DPPE, or a combination thereof;
(d) the at least one sterol is cholesterol;
(e) the lipid nanoparticle comprises about 20-60% ionizable cationic lipid, about 5-25% helper lipid, about 25-55% sterol, and about 0.5-15% PEG-modified lipid.

36-39. (canceled)

40. A pharmaceutical composition comprising the lipid nanoparticle of claim 30 and a pharmaceutically acceptable carrier or excipient.

41. A vaccine comprising one or more lipid nanoparticles of claim 30, and further comprising a pharmaceutically acceptable adjuvant,
optionally wherein the one or more lipid nanoparticles are part of a pharmaceutical composition further comprising a pharmaceutically acceptable carrier or excipient.

42. A method of inducing in a subject an immune response to a coronavirus, comprising administering to the subject the vaccine of claim 41 in an amount effective to generate the immune response.

43. The method of claim 42, wherein at least one the following applies:
(a) the immune response comprises a T cell response and/or a B cell response;
(b) the immune response comprises a neutralizing antibody response specific to the coronavirus spike protein;
(c) the immune response inhibits infection by the coronavirus and/or replication of the coronavirus in the subject;
(d) the subject is administered a single dose of the vaccine;
(e) the subject is administered two or more doses of the vaccine, optionally wherein the two or more doses are administered 14-28 days apart;
(f) each administration of the vaccine comprises a dose of about 1 μg, 3 μg, 10 μg, 25 μg, 30 μg or 100 μg.

44-48. (canceled)

49. The method of claim 42, wherein at least one of the following applies:
(a) the effective amount is a total dose of about 1-500 μg, inclusive;
(b) the vaccine is administered by intradermal injection, intramuscular injection, oral administration, intranasal administration, or intratracheal administration;
(c) the subject has been exposed to, is infected with, or is at risk of infection by the coronavirus;
(d) the subject is immunocompromised;
(e) the subject is human;
(f) the coronavirus is selected from MERS-CoV, SARS-CoV, SARS-CoV-2, and variants thereof.

50-54. (canceled)
